(19) United States
(12) Patent Application Publication
(10) Pub. No.: US 2026/0162769 A1
(43) Pub. Date: Jun. 11, 2026

(54) SYSTEMS AND METHODS FOR GENE EXPRESSION AND TISSUE OF ORIGIN INFERENCE FROM CELL-FREE DNA

(71) Applicant: The Board of Trustees of the Leland Stanford Junior University, Stanford, CA (US)

(72) Inventors: Maximilian Diehn, San Carlos, CA (US); Arash Ash Alizadeh, San Mateo, CA (US); Mahya Mehrmohamadi, Moraga, CA (US); Mohammad Shahrokh Esfahani, Mountain View, CA (US)

(21) Appl. No.: 18/706,700

(22) PCT Filed: Nov. 16, 2022
(86) PCT No.: PCT/US2022/050151

§ 371 Date: May 1, 2024

Related U.S. Application Data
(60) Provisional application No. 63/280,305, filed on Nov. 17, 2021.

(51) Int. Cl. G16B 25/10; C12Q 1/6874; C12Q 1/6881; C12Q 1/6886; G16B 30/10

(52) U.S. Cl. CPC G16B 25/10; C12Q 1/6874; C12Q 1/6881; C12Q 1/6886; G16B 30/10; C12Q 2600/158

(57) ABSTRACT

Methods are provided for non-invasively determining the expression of genes of interest by inference and the use thereof in disease classification and stratification for treatment. Disclosed methods relate to assessment of fragment length diversity cell-free DNA, such as determining promoter fragment entropy (PFE). Fragment length diversity scores may be combined with nucleosome depleted region depth to produce a metric that is indicative of gene expression. In some embodiments, the methods use only noninvasive blood draws and identify which patients will achieve durable clinical benefit from immune checkpoint inhibition, what the cancer subtype classification is, and/or what the tumor burden is. In an embodiment, the methods further comprise selecting a treatment regimen for the individual based on the analysis. Also disclosed are bait sets for enrichment of cell-free DNA from regions in proximity with transcription start sites.

### CROSS REFERENCE TO OTHER APPLICATIONS

This application claims the benefit of U.S. Provisional Application No. 63/280,305, filed Nov. 17, 2021, the contents of which are hereby incorporated by reference in its entirety.

### STATEMENT AS TO FEDERALLY SPONSORED RESEARCH

This invention was made with government support under CA188298 awarded by the National Institutes of Health. The government has certain rights in the invention.

### SUMMARY OF THE INVENTION

Described herein, in certain embodiments, are bait sets comprising a plurality of probes configured to enrich for cell-free DNA molecules from at least 5% of the genomic regions described throughout the specification. In some embodiments, the genomic regions are described in Tables 1 and 2.

In some embodiments, the plurality of probes is configured to enrich for cell-free DNA molecules from at least 5%, at least 10%, at least 20%, at least 30%, at least 40%, at least 50%, at least 60%, at least 70%, at least 80%, at least 90%, at least 95%, at least 99%, or 100% of the genomic regions in Table 1. In some embodiments, at least 20%, at least 30%, at least 40%, at least 50%, at least 60%, at least 70%, at least 80%, at least 90%, at least 95%, or at least 99% of probes in the bait set are configured to enrich for genomic regions in Table 1. In some embodiments, the plurality probes are configured to enrich for cell-free DNA molecules from at least 100, at least 500, at least 1,000, at least 1,500, or at least 2,000 genomic regions in Table 1. In some embodiments, each of the plurality of probes comprises a nucleic acid sequence of at least 50 bases, at least 70 bases, at least 80 bases, or at least 100 bases in length that has at least 95%, 99%, or 100% complementarity to a sequence of a region in Table 1. In some embodiments, the plurality of probes is configured to enrich for cell-free DNA molecules from at least 5%, at least 10%, at least 20%, at least 30%, at least 40%, at least 50%, at least 60%, at least 70%, at least 80%, at least 90%, at least 95%, at least 99%, or 100% of the genomic regions in Table 2. In some embodiments, at least 20%, at least 30%, at least 40%, at least 50%, at least 60%, at least 70%, at least 80%, at least 90%, at least 95%, or at least 99% of probes in the bait set are configured to enrich for genomic regions in Table 2. In some embodiments, the plurality probes are configured to enrich for cell-free DNA molecules from at least 500, at least 1,000, or at least 1,500 genomic regions in Table 2. In some embodiments, each of the plurality of probes comprises a nucleic acid sequence of at least 50 bases, at least 70, at least 80, or at least 100 bases in length that has at least 95%, 99%, or 100% complementarity to a sequence of a region in Table 2. In some embodiments, each of the plurality of probes comprises a nucleic acid sequence configured for hybridization capture of the cell-free DNA molecules. In some embodiments, each of the plurality of probes is at least 50 bases, at least 100 bases, or at least 200 bases in length. In some embodiments, each of the plurality of probes is no more than 500 bases, 1,000 bases, 2,000 bases, or 5,000 bases in length. In some embodiments, each of the plurality of probes is between 50 and 5,000 bases, between 100 and 4,000 bases, or between 200 and 2,500 bases, or between 100 and 500 bases in length. In some embodiments, the plurality of probes comprises at least 100, at least 500, at least 1000, or at least 4000 different probes. In some embodiments, the bait set has at most 10,000 different probes. In some embodiments, the plurality of probes collectively extend across portions of the genome that collectively are a combined size of between 0.5 MB and 2.5 MB. In some embodiments, each probe of the plurality of probes comprises a pull-down tag. In some embodiments, the pull-down tag comprises biotin.

Described herein, in certain embodiments, are methods for inferring an expression level of one or more genes of interest in a subject, the method comprising: (i) obtaining sequencing data for a plurality of cell-free DNA molecules of a subject; (ii) aligning the sequencing data for the plurality of cell-free DNA molecules to a reference genome; (iii) determining sequence length for each of the plurality of cell-free DNA molecules of the subject; (iv) calculating, for each of the one or more genes of interest, a fragment length diversity measure from cell-free DNA molecules that, when aligned to the reference genome, are within a specified distance from a transcription start site of the gene of interest; and (v) determining, by inference, a gene expression level for the one or more genes of interest based at least in part on the fragment length diversity measure for each of the one or more genes of interest. In some embodiments, the method further comprises contacting the cell-free DNA molecules of the subject with the bait set according to the present disclosure to enrich for cell-free DNA from regions within 750 base pairs of transcription start sites. In some embodiments, the fragment length diversity measure is calculated from cell-free DNA molecules in which both ends fall within 1 kb of the transcription start site for the gene of interest.

In some embodiments, the fragment length diversity measure is calculated from cell-free DNA molecules in which both ends fall within 1 kb of the transcription start site for the gene of interest. In some embodiments, the fragment length diversity measure is promoter fragment entropy. In some embodiments, the number of genes of interest is at least two, at least 5, at least 10, at least 15, or at least 25. In some embodiments, the fragment length diversity measure is promoter fragment entropy, wherein promoter fragment entropy is calculated using the equation PFE(TSS):=Eₖ[Σ_{i:1-5}P*(e_{TSS}>(1+k)×eᵢ)]. In some embodiments, the method further comprises calculating a nucleosome depleted region depth.

In some embodiments, the method further comprises calculating a nucleosome depleted region depth. In some embodiments, the method further comprising combining the calculated fragment length entropy measure with the calculated nucleosome depleted region depth to generate a metric that is indicative of the expression level of the gene of interest.

In some embodiments, the method further comprises combining the calculated fragment length entropy measure with the calculated nucleosome depleted region depth to generate a metric that is indicative of the expression level of the gene of interest. In some embodiments, steps (iv) and (v) are performed by a computer system comprising software components for data analysis as a program of instructions executable by the computer system. In some embodiments, steps (ii)-(v) are performed by a computer system comprising software components for data analysis as a program of instructions executable by the computer system. In some embodiments, steps (i)-(v) are performed by a computer system comprising software components for data analysis as a program of instructions executable by the computer system.

In some embodiments, the method further comprises: (i) obtaining a biological sample from the subject, the biological sample comprising the cell-free DNA; (ii) constructing a sequencing library from the cell-free DNA from the biological sample; and (iii) sequencing the sequencing library to obtain the sequencing data for the plurality of cell-free DNA molecules of the subject. In some embodiments, constructing the sequencing library comprises ligating adaptors to the cell-free nucleic acid molecules and enriching for nucleic acids from select regions by hybridizing a selector to the adaptor-containing molecules, thereby forming the sequencing library.

In some embodiments, constructing the sequencing library comprises ligating adaptors to the cell-free nucleic acid molecules and enriching for nucleic acids from select regions by hybridizing a selector to the adaptor-containing molecules, thereby forming the sequencing library. In some embodiments, the selector comprises or consists of a selector as described in the specification. In some embodiments, the selector is designed to enrich for cell-free DNA molecules in proximity to (e.g., within 1 kb of) one or more transcription start sites for one or more genes, wherein the genes are selected from ASCL1, CLDN3, DLL3, DNALI1, DPYSL3, EEF1A2, ESRP1, FOXA2, GRP, HOXB5, ID4, IGFBP5, IGFBPL1, ISL1, KRT19, KRT7, MMP2, NKX2-1, PCSK2, SCG3, SIX1, SYT13, SYT4, TAGLN3, and TM4SF1. In some embodiments, the selector is designed to enrich for cell-free DNA molecules in proximity to transcription start sites for at least 10%, at least 20%, at least 50%, at least 70%, at least 80%, at least 90, at least 95%, or 100% of the following genes: ASCL1, CLDN3, DLL3, DNALI1, DPYSL3, EEF1A2, ESRP1, FOXA2, GRP, HOXB5, ID4, IGFBP5, IGFBPL1, ISL1, KRT19, KRT7, MMP2, NKX2-1, PCSK2, SCG3, SIX1, SYT13, SYT4, TAGLN3, and TM4SF1.

In some embodiments, the biological sample is obtained from an individual with cancer. In some embodiments, the cancer is small cell lung cancer. In some embodiments, the cancer is non-small cell lung cancer. In some embodiments, the cancer is lung cancer or a B-cell lymphoma. In some embodiments, the subject has a tumor burden having a mixture fraction of at least 0.25, 0.5, 0.75, 1.0, 1.5, 2.0, 2.5, 5, 7.5, 10, or 15 and the sequencing data has at least 500×, 2500×, or 5000× coverage for regions comprising the transcription start sites for the one or more genes of interest.

In some embodiments, the sequencing data is obtained from a biological sample obtained prior to immune checkpoint inhibitor treatment. In some embodiments, gene expression levels for the one or more genes of interest are monitored after treatment with an immune checkpoint inhibitor. In some embodiments, the sequencing data is obtained from a biological sample that was obtained within 4 weeks of a first immune checkpoint inhibitor treatment. In some embodiments, the biological sample is a non-invasively obtained sample from blood. In some embodiments, the biological sample is a serum sample.

In some embodiments, the individual with cancer (1) is treated with an immune checkpoint inhibitor if durable clinical benefit is predicted and (2) is treated with non-immune checkpoint inhibitor therapy if durable clinical benefit is not predicted. In some embodiments, the immune checkpoint inhibitor is a PD-1 or PD-L1 inhibitor. In some embodiments, if the individual is diagnosed as having a specific cancer, said individual is then treated for said cancer.

In some embodiments, the sequencing is at a depth of at least 500×, 2000×, 2500× or 5000×.

In some embodiments, an increase in the fragment length diversity measure (e.g., promoter fragment entropy) of the gene of interest correlates with an increase in expression of the gene of interest. In some embodiments, an increase in the fragment length diversity measure (e.g., promoter fragment entropy) of the gene of interest correlates with expression of exon 1 of the gene of interest.

In some embodiments, the subject has a disease state based at least in part on (1) the fragment length diversity measure of a plurality of genes of interest or (2) the gene expression levels of the plurality of genes of interest as determined by inference from the fragment length diversity measures for the plurality of genes.

In some embodiments, the method further comprises identifying a tissue of origin for diseased tissue from the subject based at least in part on (1) the fragment length diversity measure of a plurality of genes of interest or (2) the gene expression levels of the plurality of genes of interest as determined by inference from the fragment length diversity measures for the plurality of genes. In some embodiments, the number of genes of interest is at least two, at least 5, at least 10, at least 15, or at least 25.

In some embodiments, the method further comprises: obtaining a biological sample from the subject, the biological sample comprising the cell-free DNA; constructing a sequencing library from the cell-free DNA from the biological sample; and sequencing the sequencing library to obtain the sequencing data for the plurality of cell-free DNA molecules of the subject. In some embodiments, constructing the sequencing library comprises enriching for cell-free nucleic acid molecules from select regions by hybridization capture. In some embodiments, constructing the sequencing library comprises ligating adaptors to the cell-free nucleic acid molecules and enriching for nucleic acids from select regions by hybridizing a selector to the adaptor-containing molecules, thereby forming the sequencing library. In some embodiments, the selector comprises or consists of a selector according to the present disclosure. In some embodiments, the selector comprises the bait set according to the present disclosure.

In some embodiments, the steps of the methods are performed by a computer system comprising software components for data analysis as a program of instructions executable by the computer system. In some embodiments, the steps of the methods are implemented on a computer system comprising a software component configured for analysis of data obtained by the methods. In some embodiments, the software product is tangibly embodied in a machine-readable medium, the software product comprising instructions operable to cause one or more data processing apparatuses to perform the method according to the present disclosure.

Disclosed herein, in certain embodiments, are methods for determining a fragment length diversity measure for one or more genes of interest, the method comprising: (i) obtaining sequencing data for a plurality of cell-free DNA molecules of a subject; (ii) aligning the sequencing data for the plurality of cell-free DNA molecules to a reference genome; (iii) determining sequence length for each of the plurality of cell-free DNA molecules of the subject; and (iv) calculating, for each of the one or more genes of interest, a fragment length diversity measure from cell-free DNA molecules that, when aligned to the reference genome, are within a specified distance from a transcription start site of the gene of interest.

In some embodiments, the method further comprises contacting the cell-free DNA molecules of the subject with the bait set of the present disclosure to enrich for cell-free DNA from regions within 750 base pairs of transcription start sites. In some embodiments, the fragment length diversity measure is calculated from cell-free DNA molecules in which both ends fall within 1 kb of the transcription start site for the gene of interest. In some embodiments, the fragment length diversity measure is calculated from cell-free DNA molecules in which both ends fall within 900 base pairs, within 850 pairs, within 800 base pairs, or within 750 base pairs of the transcription start site for the gene of interest. In some embodiments, the fragment length diversity measure is promoter fragment entropy, wherein fragment entropy is calculated using the equation PFE(TSS):=Eₖ[Σ_{i:1-5}P*(e_{TSS}>(1+k)×eᵢ)]. In some embodiments, the method further comprises calculating a nucleosome depleted region depth. In some embodiments, the method further comprises combining the calculated fragment length entropy measure with the calculated nucleosome depleted region depth to generate a metric that is indicative of an expression level of the gene of interest. In some embodiments, steps (iii) and (iv) are performed by a computer system comprising software components for data analysis as a program of instructions executable by the computer system. In some embodiments, steps (ii)-(iv) are performed by a computer system comprising software components for data analysis as a program of instructions executable by the computer system. In some embodiments, steps (i)-(iv) are performed by a computer system comprising software components for data analysis as a program of instructions executable by the computer system.

In some embodiments, the method further comprises obtaining a biological sample from the subject, the biological sample comprising the cell-free DNA: constructing a sequencing library from the cell-free DNA from the biological sample; and sequencing the sequencing library to obtain the sequencing data for the plurality of cell-free DNA molecules of the subject.

In some embodiments, constructing the sequencing library comprises enriching for cell-free nucleic acid molecules from select regions by hybridization capture. In some embodiments, constructing the sequencing library comprises ligating adaptors to the cell-free nucleic acid molecules and enriching for nucleic acids from select regions by hybridizing a selector to the adaptor-containing molecules, thereby forming the sequencing library. In some embodiments, the selector comprises or consists of a selector as described in the specification. In some embodiments, the selector comprises or consists of the bait set according to the present disclosure. In some embodiments, the selector is designed to enrich for cell-free DNA molecules in proximity to (e.g., within 1 kb of) one or more transcription start sites for one or more genes, wherein the genes are selected from ASCL1, CLDN3, DLL3, DNALI1, DPYSL3, EEF1A2, ESRP1, FOXA2, GRP, HOXB5, ID4, IGFBP5, IGFBPL1, ISL1, KRT19, KRT7, MMP2, NKX2-1, PCSK2, SCG3, SIX1, SYT13, SYT4, TAGLN3, and TM4SF1. In some embodiments, the selector is designed to enrich for cell-free DNA molecules in proximity to transcription start sites for at least 10%, at least 20%, at least 50%, at least 70%, at least 80%, at least 90, at least 95%, or 100% of the following genes: ASCL1, CLDN3, DLL3, DNALI1, DPYSL3, EEF1A2, ESRP1, FOXA2, GRP, HOXB5, ID4, IGFBP5, IGFBPL1, ISL1, KRT19, KRT7, MMP2, NKX2-1, PCSK2, SCG3, SIX1, SYT13, SYT4, TAGLN3, and TM4SF1.

In some embodiments, the biological sample is obtained from an individual with cancer. In some embodiments, the cancer is a cancer described in the specification. In some embodiments, the cancer is small cell lung cancer. In some embodiments, the cancer non-small cell lung cancer. In some embodiments, the cancer is lung cancer or a B-cell lymphoma.

In some embodiments, the sequencing data is obtained from a biological sample obtained prior to immune checkpoint inhibitor treatment. In some embodiments, the method further comprises calculating, for each of the one or more genes of interest, a fragment length diversity after treatment with an immune checkpoint inhibitor. In some embodiments, the sequencing data is obtained from a biological sample that was obtained within 4 weeks of a first immune checkpoint inhibitor treatment. In some embodiments, the biological sample is a non-invasively obtained sample from blood. In some embodiments, the biological sample is a serum sample. In some embodiments, the individual with cancer (1) is treated with an immune checkpoint inhibitor if durable clinical benefit is predicted and (2) is treated with non-immune checkpoint inhibitor therapy if durable clinical benefit is not predicted. In some embodiments, the immune checkpoint inhibitor is a PD-1 or PD-L1 inhibitor. In some embodiments, the individual is diagnosed as having a specific cancer, said individual is then treated for said cancer.

In some embodiments, the sequencing is at a depth of at least 500×, 2000×, 2500× or 5000×.

In some embodiments, an increase in the fragment length diversity measure (e.g., promoter fragment entropy) of the gene of interest correlates with an increase in expression of the gene of interest. In some embodiments, the increase in the fragment length diversity measure (e.g., promoter fragment entropy) of the gene of interest correlates with expression of exon 1 of the gene of interest. In some embodiments, the method further comprises identifying a tissue of origin for diseased tissue from the subject based at least in part on (1) the fragment length diversity measure of a plurality of genes of interest or (2) the gene expression levels of the plurality of genes of interest as determined by inference from the fragment length diversity measures for the plurality of genes. In some embodiments, the number of genes of interest is at least two, at least 5, at least 10, at least 15, or at least 25. In some embodiments, one or more steps are implemented on a computer system comprising a software component configured for analysis of data obtained by the methods.

Disclosed herein, in certain embodiments, is a software product tangibly embodied in a machine-readable medium, wherein the software product comprising instructions operable to cause one or more data processing apparatuses to perform the method according to the present disclosure.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1. Correlation of gene expression and cell-free DNA molecular features. (a) Chromatin accessibility footprints can be traced back to the tissue of origin. Open chromatin is subject to nuclease digestion resulting in decreased sequencing coverage depth, measured by nucleosome depletion rate (NDR), and fragment length diversity, measured by promoter fragmentation entropy (PFE). In this cartoon, lung epithelial cells exhibit very low expression of MS4A1 (CD20) but high expression of NKX2-1 (TTF1). The cfDNA fragments of a lung cancer patient consist of normal primarily hematopoietic cfDNA fragments mixed with fragments derived from lung adenocarcinoma cells undergoing apoptosis. Because the lung epithelial cell compartment has a lower coverage (NDR) and higher fragment length diversity (PFE) for NKX2-1 fragments, the resulting mixture shows similar changes with the net effect dependent on the total amount of circulating tumor-derived fragments. B-cells, on the other hand, highly express MS4A1 (CD20) with a very low expression level of NKX2-1. Accordingly, the cfDNA fragments of a B-cell lymphoma patient consist of normal cfDNA fragments admixed with B-cell derived ctDNA with overrepresentation of MS4A1 resulting in lower coverage and higher diversity of cfDNA fragment length values at the transcription start site (TSS). (b) A heatmap depicts cfDNA fragment size densities at transcription start sites (TSS) across the genome in an exemplar plasma sample profiled by high-depth whole-genome sequencing (˜250×). The X-axis depicts cfDNA fragment size, while the rows of the heatmap capture fragment density as ordered by gene expression profile (GEP) in blood leukocytes assessed by RNA-Seq using transcripts per million (TPM, right). Each row corresponds to one meta-gene encompassing the TSSs of 10 genes when ranked by a reference PBMC expression vector. The data are normalized column-wise for each cfDNA fragment size bin. Corresponding PFE, NDR, and TPM levels are depicted for each bin in dot plots on the right. (c) A scatter plot depicts the relationship between plasma cfDNA PFE versus leukocyte RNA expression levels (TPM), as in panel (b). (d) Pearson correlations between individual cfDNA fragment features (PFE. NDR, OCF, WPS, and MDS) and leukocyte gene expression levels; OCF: orientation-aware cfDNA fragmentation; WPS: windowed protection score; MDS: motif diversity score. The error bars depict the 95% confidence intervals resulted from 500 bootstrap replicates (resampling with replacement of gene groups). (e) The correlation between leukocyte gene expression and each of two leading cfDNA features (PFE and NDR) as a function of distance to the TSS center. The orange curve shows the higher average correlation for cfDNA PFE than NDR's correlation at all distances from the TSS center. The dotted lines correspond to the concordance measure when evaluated on the shorn leukocyte DNA from a matched blood PBMC sample. (f) Relationship between PFE of a non-small cell lung cancer (NSCLC) signature and cfDNA sample status (non-cancer vs cancer) and across stages. The PFE monotonically increases from non-cancer to later stages patients with NSCLC (Jonckheere's trend test P=0.0005). (g) Relationship between PFE of a gene set with low expression in NSCLC (and high in PBMC) and cfDNA sample status (non-cancer vs cancer) and across stages. The PFE of this set is not associated with disease status (normal vs cancer) or disease stage (Jonckheere's trend test P=0.54). (h) Effect of sequencing depth (X-axis) on the correlation of cfDNA PFE and NDR with gene expression (Y-axis). For each down-sampled depth, three replicates were generated, and the shaded area illustrates three standard deviation above and below the mean.

FIG. 2. Fragment size entropy in relation to gene structure informs gene expression inferences from whole exome cfDNA profiling. (a) Heatmap depicts the mean normalized Shannon entropy of cfDNA fragment size distributions for 18.131 individual protein-coding genes when sorted by their expression in blood PBMC leukocytes, across a 20 Kb region flanking each TSS when sliding a 2 kb window. The heat illustrates the normalized entropy (normalization to the average entropy over the start to end of this 20 Kb region). The maximum heat (shown by light yellow) occurs at the TSS for the highly expressed genes (top), whereas the contrast is lower for genes with lower expression (bottom). The underlying data are the deep whole-genome cfDNA profile from FIG. 1b. (b) A summary representation of the heatmap in panel a. Each column reflects a window position across the TSS, and is summarized by a histogram depicting the deviation of Shannon from the window centered at the TSS (position 0). (c) Concordance analysis using Pearson correlation between individual gene expression and PFEs when calculated in TSS, exon 1, intron 1, etc. Each dot corresponds to one cfDNA sample profiled deeply by whole genome sequencing (n=3). This analysis shows that after exon 1, there is a significant drop in the correlation. (d) Genes known to be highly expressed in SCLC tumors by RNA-Seq (n=118 genes from 81 tumors) exhibit significantly higher PFE in cfDNA samples from patients with SCLC (n=11, pink dots) than healthy adult control subjects (n=28, brown dots; P=3.94E-5), as profiled by deep (˜2000×) whole exome sequencing. See Methods and FIG. 7g. (e) As in d, but showing significantly lower average PFE in cfDNA of SCLC patients, when considering 20 genes known to be lowly expressed in SCLC tumors but highly expressed in PBMCs by RNA-Seq (P=0.02). (f) Differentially expressed genes (DEGs) associated with SCLC, identified directly from cfDNA using PFE analysis. Volcano plot depicts genes inferred to be more highly expressed in 11 cfDNA samples SCLC cases (pink dots, n=620), or in 28 cfDNA samples from healthy adult controls (brown dots, n=596). DEGs were determined by considering the magnitude of mean PFE difference between groups (X-axis; 10.11) and the false-discovery rate (Q<0.05) from t-tests between groups. These two sets of genes discovered noninvasively from cfDNA as differentially expressed in SCLC, were then assessed for expression in primary SCLC tumors in panels (g-h). The box-and-whisker plots depict the mean RNA expression levels (Y-axis, TPM) observed for the SCLC high (g) and SCLC low (h) gene sets, when comparing RNA-Seq in SCLC tumors (n=81, pink dots) versus healthy PBMCs (n=13, brown dots).

FIG. 3. EPIC-Seq design and workflow. (a) The schema depicts the general workflow of EPIC-Seq, starting with cfDNA extraction from plasma, library preparation and capture of TSS of genes of interest, high-throughput sequencing of enriched regions, and finally, cfDNA fragmentation analysis followed by machine learning models for prediction of expression at each TSS and classification of the specimen. (b-c) The volcano plots depict differentially expressed genes, as informative for histological classification in non-small cell lung cancer subtypes (lung adenocarcinoma [LUAD] vs lung squamous cell carcinoma [LUSC] from the TCGA), and in cell-of-origin classification of diffuse large B-cell lymphoma (ABC vs GCB from Schmitz, R. et al. Genetics and Pathogenesis of Diffuse Large B-Cell Lymphoma. N Engl J Med 378, 1396-1407 (2018)). Genes highlighted in colors other than grey were selected for TSS capture in EPIC-Seq, after censoring genes with high expression in blood leukocytes (see Methods). (d) NKX2-1, encoding TTF1, known to be highly expressed in NSCLC-LUAD tumors, exhibits significantly higher predicted expression in cfDNA of patients with LUAD by EPIC-Seq (LUAD vs others Wilcoxon test P=5.7E-6). (e) MS4A1, encoding CD20, known to be a marker of DLBCL tumors, exhibits significantly higher predicted expression in cfDNA of patients with DLBCL by EPIC-Seq (DLBCL vs others Wilcoxon test P=5.44E-9). Box-and-whisker plots depict predicted expression levels in individual samples profiled by EPIC-Seq (dots), with boxes spanning the inter-quartile range; the median is horizontally marked with a line in each box, and whiskers span the 1.5 IQRs in each patient cohort.

FIG. 4. Application of EPIC-Seq for lung cancer detection and histological classification. (a) Receiver-Operator Curve (ROC) capturing performance of the EPIC-Lung classifier for distinguishing lung cancers from others in leave-one-batch-out analyses (AUC=0.91). The 95% confidence interval of the AUC is calculated using 2000 bootstrap replicates. (b) Relationship between EPIC-Lung scores and NSCLC disease Stage, with test for trend measured by Jonckheere's test (P=0.08). Box-and-whisker plots depict the EPIC-lung classifier score in individual samples profiled by EPIC-Seq (dots), with boxes spanning the inter-quartile range; the median is horizontally marked with a line in each box, and whiskers span the 1.5 IQRs in each disease stage group. (c) Sensitivity analysis of the EPIC-Lung classifier at 95% specificity. Patients are grouped based on bins of mean circulating tumor allele fraction (<1%, 1-5% and >5%), estimated by CAPP-Seq on the same samples. Sensitivity improves as ctDNA AF increases with ˜33% of patients detectable when AF<1%. The error bars depict the 95% confidence interval of the sensitivity values resulted from 500 bootstrap replicates. (d) ROC curve of the LUAD vs LUSC classifier when tested in a leave-one-out framework (AUC=0.90, 95%-CI [0.83-0.97]). (e) Coefficients of the NSCLC histology classifier, with positive and negative coefficients favoring LUAD and LUSC, respectively. The coefficients are significantly associated with prior knowledge when comparing their magnitude and polarity by t-test (P=0.033). Box-and-whisker plots are defined as in (b) and are resulted from 67 coefficient sets from classifiers trained in the leave-one-out cross-validation step. (f) Accuracy of the histology classifier as a function of tumor ctDNA fraction as measured by CAPP-Seq. The (optimal) threshold for classification is determined in the leave-one-out framework by minimizing the average of class-conditional errors. The error bars are defined as in (a). (g) Application of inferred gene expression values from EPIC-Seq in predicting response to immune-checkpoint inhibitors within 4 weeks of treatment initiation. (h) ROC curve of the EPIC-Seq lung dynamics score calculated in panel g distinguishes patients with durable clinical benefit (DCB) vs those with no durable benefit (NDB) within 6 months (AUC=0.93, 95% CI [0.78-1]). (i) Prognostic value of EPIC-Seq lung dynamics scores in Kaplan-Meier analysis of Progression Free Survival in the patients treated with to immune-checkpoint inhibitors (log-rank P-value=0.0003; HR=11.86). Patients are stratified by the median dynamics score, with higher scores associated with higher expression in lung cancer genes and therefore, worse outcome.

FIG. 5. Application of EPIC-Seq for DLBCL detection. (a) Receiver-Operator Curve (ROC) analyses capture performance of the EPIC-DLBCL classifier for distinguishing lymphomas from others. Red and blue curves depict performance in performance in the validation cohort (AUC=0.96), versus leave-one-batch-out cross-validation analyses of the training cohort (AUC=0.92), respectively. (b) Relationship between EPIC-Seq DLBCL classifier scores and clinical prognostic scores as measured by the Revised International Prognostic Index (R-IPI; Jonckheere's trend test P=4E-4). Box-and-whisker plots depict the EPIC-DLBCL score in individual samples profiled by EPIC-Seq (dots), with boxes spanning the inter-quartile range; the median is horizontally marked with a line in each box, and whiskers span the 1.5 IQRs. (c) Sensitivity analysis at 95% specificity for EPIC-DLBCL classifier. Similar to the EPIC-Lung cancer classifier, sensitivity significantly improves as a function of ctDNA level. The error bars depict the 95% confidence interval of the sensitivity values resulted from 500 bootstrap replicates. (d-e) Change of ctDNA disease burden in response to treatment and during clinical progression in two DLBCL patients with GCB (d) and ABC (e) cell-of-origin. Shown is the radiographic response as measured by PET/CT MTV (first row y-axis), ctDNA mean AF measured by CAPP-Seq (second row y-axis), and the EPIC-seq lymphoma score (third row y-axis) over serial, pre- and post-therapy time points (x-axis).

FIG. 6. Application of EPIC-Seq for DLBCL cell-of-origin classification. (a) Relationship between DLBCL cell-of-origin EPIC-Seq GCB scores and mutation-based GCB scores as measured by CAPP-Seq (Spearman ρ=0.75, P=1e-5). Data were smoothed by 3-patient bins after sorting by CAPP-Seq scores before correlation analysis. (b) Relationship between EPIC-Seq GCB scores from cfDNA and tumor tissue clinical classification by Hans immunohistochemical algorithm (Wilcoxon P-value=0.001). Box-and-whisker plots depict the EPIC-Seq GCB score in individual samples profiled by EPIC-Seq (dots), with boxes spanning the inter-quartile range; the median is horizontally marked with a line in each box, and whiskers span the 1.5 IQRs. (c) Prognostic value of EPIC-Seq cell-of-origin scores in Kaplan-Meier analysis of Event Free Survival in DLBCL (log-rank P-value=0.013). Patients are stratified by the median EPIC-COO score, with higher scores associated with GCB and lower levels with ABC subtype. (d) Concordance analysis between EPIC-Seq COO score and RNA-based scores (from matched tumor biopsy) for a cohort of 12 patients with DLBCL. Each dot represents one patient, with the X-axis showing the GCB-score from RNA-Seq and Y-axis showing the EPIC-Seq GCB score. The two scores exhibit reasonably strong correlation (r=0.84, P=0.0006). (e) Prognostic value of individual genes profiled by EPIC-Seq and Event-Free Survival, as measured by Z-scores from univariate Cox proportional hazard models. For genes with multiple TSS regions. Z-scores were combined using Stouffer's method (Gentles, 2015). After correcting for multiple hypothesis testing, only LMO2 (red) remains significant significantly associated with favorable DLBCL outcome. Dotted lines represent the significance threshold for Bonferroni-corrected P-values of 0.05. (f) Forest-plot depicts multivariable Cox proportional hazard model results for event-free survival (EFS). After adjusting for IPI and ctDNA allele fraction, only the distal TSS for LMO2 remains significantly prognostic for EFS (P=0.005).

FIG. 7. Fragment length density at the transcription start sites varies with gene expression. (a) A heatmap of fragment length densities across 1,748 groups of genes (similar to FIG. 1a). Three regions R1 (100-150 bps), R2 (151-210 bps), and R3 (211-300 bps) show enrichment in either high or low expression gene groups. (b) The percent of fragments within each region defined in panel (a) in the deep whole-genome sample across deciles of the reference PBMC gene expression vector, i.e., 10 groups of genes when sorted by their expression values in PBMC. Highly expressed genes include fewer monosome fragments, indicating a wider distribution and thereby a higher PFE. (c) Fraction of fragments within the three regions, R1-R3, for exons vs introns vs TSS sites for the top (and bottom) 2000 genes as ranked by expression. The fraction of monosomal fragments within TSS regions is substantially lower than within intronic and exonic regions (63.5% at TSS vs ˜71% at non-TSS). Pearson's Chi-Squared goodness-of-fit tests resulted in the following test statistics (TSS vs Exon: G=62,133 [P<2.2E-16]; TSS vs Intron: G=84,110 [P<2.2E-16]). (d) Fraction of fragments falling within each region (R1, R2, and R3) for mutant cfDNA fragments and their wildtype counterparts. Each dot represents one tuple (variant-patient) and the connecting lines indicate the paired mutant-wildtype status. These results show that the mutant cfDNA fragments are enriched for R1 and R3 while wildtype fragments are enriched in R2. (e) A contour plot capturing the relationship between expression level (depicted by heat) as a function of two cfDNA fragmentomic features used in the gene inference model: PFE and NDR. (f) ROC analysis of a ‘NSCLC Score’ for noninvasively distinguishing patients with NSCLC from healthy controls (AUC=0.76). The genes comprising this score were first defined from external RNA-Seq profiling data of primary NSCLC tumor tissues and blood samples, allowing subsequent calculation of their corresponding PFE in cfDNA samples profiled by WGS for independent NSCLC cases and healthy controls. (g) A schematic for the analyses performed for FIGS. 2d-h. (h) Sample-level ‘SCLC Score’ from deep whole exome analysis of cfDNA and associated diagnostic performance. As in the exercise for NSCLC depicted in panel f, the genes comprising this SCLC score were first defined from external RNA-Seq profiling data of primary SCLC tumor tissues and blood samples. The corresponding PFEs (as the difference between the overall PFE level of top and bottom gene signatures) were subsequently calculated in cfDNA samples we profiled by deep WES for independent SCLC cases and healthy controls. Using these scores, an AUC of 0.9 was achieved in distinguishing cases from controls. (i) The Venn diagram of SCLC high genes identified in cfDNA (whole exome profiling) and tumor biopsy (RNA-Seq transcriptome profiling), with significance of overlap assessed by hypergeometric test.

FIG. 8. Ensemble model accurately predicts gene expression in validation samples. (a) The scatterplot of the predicted vs a population-averaged gene expression across 1,748 groups of genes. The underlying data are from a merged cfDNA ‘meta-sample’ (pooled from merger of 27 healthy subjects profiled by relatively shallow WGS), achieving a correlation of 0.9 in initial validation. (b) The meta sample from panel (a) was used to assess model performance, when considering TSS-level expression values without gene grouping (n=1), as well as scenarios with 2, 3, 5 and 10 genes per group. The Pearson correlation between observed expression in PBMC versus predicted expression from our model (combining PFE and NDR) is shown in green bars. This correlation substantially improves as number of genes per group increases. The Pearson correlation values between observed gene expression and those predicted by NDR or PFE expression are shown in blue and green bars, respectively. (c) Scatterplot depicts predicted versus observed gene expression measurements across 1,748 groups of genes (dots), when comparing expression measurements by RNA-Seq on matched PBMC (x-axis) against plasma cfDNA inferences (y-axis), for a validation sample from a healthy adult that we also profiled by deep WGS (˜200×). This achieved a Pearson correlation of 0.86. (d) Similar to panel c, but for a second healthy adult control subject also profiled for validation, by deep WGS of cfDNA and matched RNA-Seq of PBMC (Pearson r=0.91). (e-f) The same analysis as in panels (a-b) for a meta whole genome sample generated from healthy subjects from Zviran et al. (g) The whole genome samples (depth ˜20-40×) from Zviran et al, were used with every ten genes grouped and the concordance between model-predicted expression and PBMC expression are evaluated using Pearson correlation (i.e., each dot is one subject). The non-cancer samples show a significantly higher correlation with normal PBMC than lung cancer cases (Wilcoxon P=0.018). (h) The ichorCNA tumor fraction estimates of the lung cancer cases in panel f are used to compare with the correlations in panel f. As shown in a scatterplot, as tumor fraction increases, the correlation decreases (r=−0.69, P=0.00052).

FIG. 9. Cohorts and cell-free DNA samples profiled by EPIC-seq in this study, including Cancer Cases and Control Subjects. (a) Schema depicts the full set of specimens profiled by EPIC-Seq (n=373), including those meeting Quality Control (QC) criteria (n=352, 95%). A subset of samples were used for the initial gene expression model tuning (n=2) and TSS filtering (n=21). The remaining 329 samples were profiled by EPIC-Seq to address disease-specific questions, including utility for cancer detection, classification of histology and cell-of-origin, and response monitoring. These included 252 samples (76.6%) from 226 subjects that comprised our Discovery/Training cohort (large light purple rectangle), as well as subsequent profiling of a Validation Cohort of 77 samples (23.4%) from 69 subjects, after models were ‘locked down’ (large light green rectangle). A subset of 22 NSCLC patients where a pair of serial blood samples were monitored for ICI response (to allow comparisons of both EPIC-Seq and CAPP-Seq and assess biological plausibility), but this exercise was not subject to any model training. No samples were shared between Training and Validation exercises, with all models locked down before independent validations. Four healthy subjects (4.5%) provided more than one cfDNA specimen with one used for Training and the second for Validation. (b) Distribution of demographic, clinical, anatomic, and pathological variables for subjects profiled by EPIC-Seq. Tabulated are the relevant indices for cancer cases (235 blood samples 201 patients), including NSCLC patients (light blue; 109 blood samples from 87 patients). DLBCL patients (light orange; 126 blood samples from 104 patients), and non-cancer control subjects (gray; 94 blood samples from 87 adults).

FIG. 10. Concordance between EPIC-Seq measurements and established NSCLC risk factors including metabolic tumor burden, ctDNA level, and ctDNA response. (a) Concordance between EPIC-lung score and metabolic tumor volume (MTV), as measured by Spearman correlation (ρ=0.67; P=0.04). (b) Concordance between EPIC-lung score and the ctDNA mean allele fractions as measured by CAPP-Seq, evaluated using Spearman correlation (ρ=0.5; P=3E-5). (c) Relationships between genetic versus epigenetic molecular responses to Immune Checkpoint Inhibitor (ICI) therapy in advanced NSCLC. Scatterplot compares molecular responses measured noninvasively by CAPP-Seq (x-axis; fold change, Log 10) and EPIC-Seq (lung dynamics score; y-axis) using serial plasma profiling before and after ICI therapy. The two orthogonal measures show moderate but significant correlation (r=0.53, P=0.012).

FIG. 11. Concordance between EPIC-Seq measurements and established DLBCL risk factors impacting outcome, including metabolic tumor volume, ctDNA level, and Cell-of-Origin. (a) The boxplots illustrate the two groups of patients stratified by their metabolic tumor volumes (>220 vs <220 mL; Wilcoxon P=0.015). (b) Similar to panel a, but for the DLBCL Validation Cohort. (c) Concordance between EPIC-DLBCL scores and ctDNA mean allele fractions (from CAPP-Seq), evaluated using Spearman correlation (ρ=0.66; P<2E-16). (d) The EPIC-DLBCL model is applied to the cfDNA profiles of 13 samples from two DLBCL patients (DLBCL002 [ABC] and DLBCL007 [GCB]). The concordance between the resulting scores and the ctDNA mean allele fractions is evaluated by Spearman correlation (ρ=0.79; P=0.004). (e) Relationship between DLBCL cell-of-origin EPIC-Seq GCB scores and mutation-based GCB scores as measured by CAPP-Seq in the validation set (Spearman ρ=0.64, P=0.01). Each dot represents one sample (related to FIG. 6a). (f) Relationship between EPIC-Seq GCB scores from cfDNA and matched tumor tissue classification by routine Hans immunohistochemical algorithm in the validation set (Wilcoxon P=0.001; related to FIG. 6b). (g) Relationship between EPIC-Seq GCB scores from cfDNA and tumor classification by RNA-seq of paired tumor tissue (Jonckheere's trend test. P=0.015). Box-and-whisker plots depict the EPIC-Seq GCB score in individual samples profiled by EPIC-Seq (dots), with boxes spanning the inter-quartile range; the median is horizontally marked with a line in each box, and whiskers span the 1.5 IQRs. (h) The Kaplan-Meier curves of EFS of the patients when labeled by the Hans algorithm. The non-GCB group contains both Non-GCB and Unknown. (i) The violin plot shows the distributions of Cox Proportional Hazard Model Z-scores when genes are grouped according to their effects on outcome (measured as EFS) in three prior tumor studies.

FIG. 12. The bait set according to the present disclosure was used to enrich cell-free DNA samples. More specifically, the bait set was used to perform EPIC-seq profiling of plasma cell-free DNA from healthy controls (n=7), patients with chronic lymphocytic leukemia (n=3), and patients with diffuse large B-cell lymphoma (n=3). An exemplary analysis focused on three genes: CD5, CD20 and CD19.

FIG. 13. The bait set was used to enrich cell-free DNA samples. More specifically, the bait set was used to perform EPIC-seq profiling of plasma cell-free DNA from three healthy individuals. Cell-free RNA sequencing was also performed on matched time points of the same individuals. The PFE values calculated using the EPIC-seq pipeline were then compared with the RNA expression levels from cfRNA.

FIG. 14. Effect of preanalytical factors on fragment size entropy and effect of GC-content correction on expression model performance. (a) The concordance between PFE values for three healthy controls profiled by EPIC-Seq using paired Streck BCT and K2EDTA tubes. A Pearson correlation of 0.94 was observed between tube types. (b) Effect of time on the bench (i.e., in days) on the PFEs in a cohort of plasma cfDNA samples. (c) Effect of additional PCR cycles on PFE. Here we profiled 4 healthy control cfDNA samples by the CAPP-Seq lung cancer selector when 3 additional PCR cycles were included to study their effect. A Pearson correlation of 0.95 was observed between standard conditions versus those incorporating additional PCR cycles. (d) Effect of correction for GC-content of TSS regions on gene expression model accuracy. Four scenarios were studied when correcting features using the GC values for NDR and PFE: PFE alone corrected. NDR alone corrected, both corrected, and neither corrected. The correction was performed using a LOESS function with a span of 0.5. Two healthy control cfDNA samples were profiled by deep whole genome sequencing. For these two subjects, we also profiled the matched PBMC by RNA-Sequencing. We then compared the predicted values from cfDNA against observed values from RNA-Seq for each of the different GC-correction scenarios and tested concordance. The concordance was evaluated using three metrics: Pearson correlation, Spearman correlation, and root-mean-square error (RMSE). When considering both cfDNA samples, none of the four GC-correction approaches seemed to consistently improve correlations or reduce associated error profiles. (e) Whole exome profiling of small cell lung cancer samples in FIG. 2 are used to investigate association between PFEs and copy number aberrations. We first determined genes with PFE significantly higher in SCLC cfDNA samples (n=11) compared with healthy control cfDNA samples (n=28) (‘High’ PFE). Similarly, we determined genes with significantly lower PFEs in SCLC cfDNA samples (‘Low’ PFE). Then, the copy number states (CNS) corresponding to all genes were identified by overlapping copy number profiles from CNVkit with the genomic coordinates of the first exons. The CNS values were then dichotomized into (i) amplification vs no-amplification and (ii) deletion vs no-deletion. Next, we summarized these by contingency tables for (i) vs PFE levels (top table) and (ii) vs PFE levels (bottom table). Finally, the association between the two was examined via Fisher's exact test, which showed insignificant associations in both tests (P=0.97 and P=0.17; for amplifications and deletions, respectively).

FIG. 15. Mechanistic model and gene detection sensitivity with various parameters. (a) The cartoon shows four scenarios considered in our simulations: (i) protected, meaning that nucleosomes are well-positioned and are all present, (ii) one nucleosome-free position is present. (iii) two nucleosome-free positions are present and (iv) three nucleosome-free positions are present. (b) The density plots show the results of generating fragment lengths via the model described in panel a. Three panels correspond to scenarios (ii-iv) vs (i) in a. (c) A varying mixture parameters is considered and its effect on the entropy for three different coverages: 500×, 2500× and 5000×. (d) A summary of panel c for active gene detection sensitivity while achieving a specificity of 85%. The error bars are from the sensitivities calculated using the ‘ci.se’ function in R pROC package. The colors correspond to three different coverages in panel c.

### DETAILED DESCRIPTION OF THE INVENTION

Profiling of circulating tumor DNA (ctDNA) in the bloodstream shows promise for non-invasive cancer detection and classification. While chromatin fragmentation features in cell-free DNA (cfDNA) have previously been explored, current fragmentomic methods require high concentrations of tumor-derived DNA and are limited by insufficient resolution to infer individual gene expression. Here, promoter fragmentation entropy (PFE) at transcription start sites (TSS) is disclosed as a novel epigenomic cfDNA feature strongly correlated with RNA expression levels. Also disclosed is that residual fragmentation entropy within first exons can be measured by whole exome cfDNA profiling in lung cancers, enabling noninvasive identification of gene expression matching corresponding tissue specimens. PFE is complementary to other fragmentomic features in predicting gene-specific transcription levels and has advantages over them. We leverage these insights within EPIC-Seq, a method for high-resolution cancer detection and tissue-of-origin classification from cfDNA that extracts features of chromatin fragmentation using targeted sequencing from promoters of genes of interest. Profiling329 blood samples from 201 cancer patients and 87 healthy adults, we demonstrate the ability of EPIC-Seq to infer gene expression at the level of individual TSS. We describe the utility of this approach for noninvasive classification of subtypes of lung carcinomas and diffuse large B-cell lymphomas, and for noninvasive cancer detection purposes. Finally, by applying EPIC-Seq to serial blood samples from patients treated with PD-(L)1 immune checkpoint inhibitors, we show that gene expression profiles inferred by EPIC-Seq after a single infusion are correlated with clinical response. Our results suggest that EPIC-Seq could augment current personalized profiling efforts, enabling noninvasive, high-throughput tissue-of-origin characterization with diagnostic, prognostic, and therapeutic potential.

Cell-free DNA (cfDNA) molecules that circulate in blood plasma largely arise from chromatin fragmentation accompanying cell death during homeostasis of diverse tissues throughout the body (Jahr, 2001; Lo, 2010; Heitzer (2020)). Accordingly, cfDNA profiling has established clinical utility for detection of tissue rejection after solid organ transplantation, noninvasive prenatal testing of fetal aneuploidy during pregnancy, and noninvasive tumor genotyping, as well as early evidence of utility for detection of diverse cancer types (Newman, 2014; Phallen, 2017; Cohen, 2018; Cristiano, 2019; Heitzer, 2019; Van Opstal, 2018; Fan, 2012; Knight, 2019). For each of these applications, current liquid biopsy testing approaches have largely relied on germline or somatic genetic variations in the sequence of cfDNA molecules, as relevant for diagnosis of pathology in the tissue of interest. Indeed such variations in genetic sequences can be highly informative for biopsy-free tumor genotyping of circulating tumor DNA (ctDNA) and for monitoring of disease burden, with potential utility for diagnosis and early cancer detection (Chabon, 2020; Chaudhuri, 2017; Lennon, 2020; Zviran, 2020).

Despite the many applications of cfDNA profiling for the noninvasive detection of mutations in the blood, even in cancers with a high tumor mutation burden and even in patients with high disease burden, most cancer-derived fragments are generally unmutated. Accordingly, the ability to interrogate these cfDNA fragments, for example, as might inform the tissue of origin of unmutated molecules using epigenetic features could have broad utility. For example, such approaches could be useful for detection of tissue injury without associated genetic lesions (Lo, 1998; Snyder, 2011; Lehmann-Werman, 2016; Jiang, 2018; Sun, 2019; Sadeh, 2021), as well as for classification of cancer entities and molecular subtypes. Since circulating cfDNA molecules are primarily nucleosome-associated fragments, they reflect the distinctive chromatin configuration of the nuclear genome of the cells from which they derive (Lui, 2002; Fleischhacker, 2007; Ramachandran, 2017). Specifically, genomic regions densely associated with nucleosomal complexes are generally protected against the action of intracellular and extracellular endonucleases, while open chromatin regions are more exposed to such degradation (Snyder, 2016).

Accordingly, several studies have recently identified specific chromatin fragmentation features across the genome as potentially useful for classification of tissue of origin by cfDNA profiling. These ‘fragmentomic’ features include a decrease in depth of sequencing coverage (Ivanov, 2015; Ulz, 2016; Wu, 2019; Jiang, 2015) and disruption of nucleosome positioning (Snyder, 2016) near transcription start sites (TSSs). Separately, several studies have shown that the length of cfDNA fragments can also inform tissue of origin, including tumor derivation, even when considered agnostic to genomic location or relation to gene promoters. For example, tumor-derived molecules bearing somatic variants tend to be shorter than their wild-type counterparts (Jiang, 2015; Underhill, 2016; Mouliere, 2018; Ulz 2019) and can be useful for distinguishing somatic variants that are tumor-derived from those arising from circulating leukocytes during clonal hematopoiesis (Chabon 2020).

Despite these advances, current fragmentomic methods, including those relying on relatively shallow whole genome sequencing (WGS) do not fully harness the contributions of various tissues to the circulating DNA pool. Separately, current fragmentomic techniques do not provide adequate genomic depth and breadth to enable gene-level resolution. Indeed, even when considering groups of genes, such fragmentomic methods only perform reasonably well for inferring gene expression at high circulating tumor DNA levels. Accordingly, fragmentomic methods for inferring gene expression are largely limited to patients with very high tumor burden generally observed in advanced disease.

We addressed these limitations by evaluating additional cfDNA fragmentation features for the purposes of predicting gene expression. We reasoned that by profiling cfDNA fragmentation in important regions at high resolution, key fragmentomic features could capture gene-level associations with expression levels across the genome and could inform accurate statistical models for predicting transcriptional output. If this hypothesis is indeed correct, then targeted deep sequencing of informative genomic regions could overcome the limitations of prior WGS approaches and allow for profiling cfDNA fragmentation at high resolution, which would in turn facilitate gene-level analyses. Here we describe a new cfDNA fragmentation feature that enables prediction of gene expression for individual genes. We leverage this observation to develop EPigenetic expression Inference from Cell-free DNA Sequencing (EPIC-Seq), a novel method for analyzing gene expression based on cfDNA fragmentomics. We then applied EPIC-Seq to classify histology of Non-Small Cell Lung Cancer [NSCLC], to distinguish molecular subtypes in Diffuse Large B-Cell Lymphoma [DLBCL], assess responses to immunotherapy, and to evaluate the prognostic value of individual genes for survival outcomes.

### Results

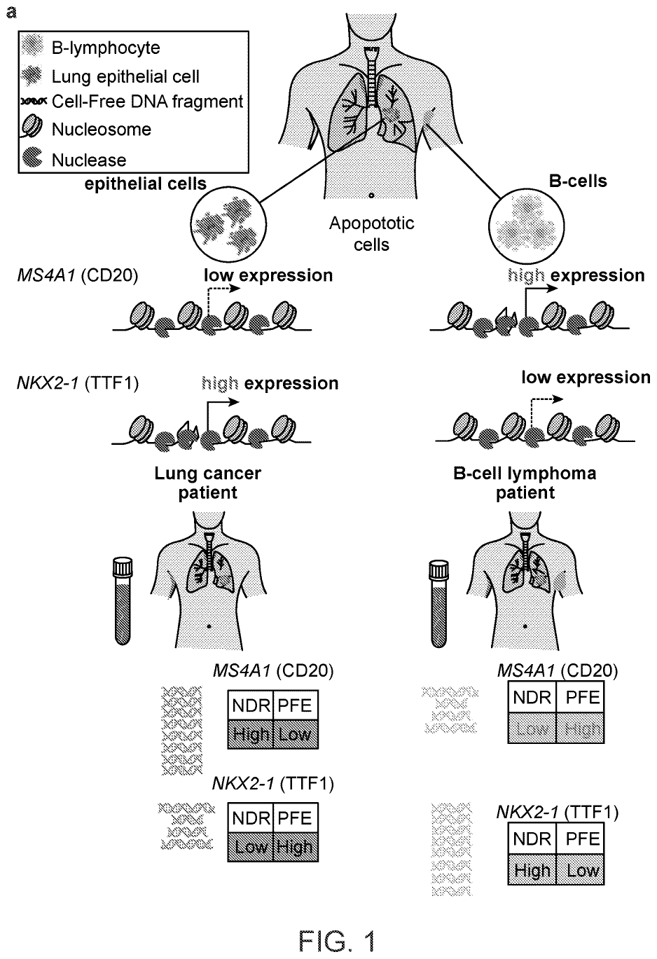

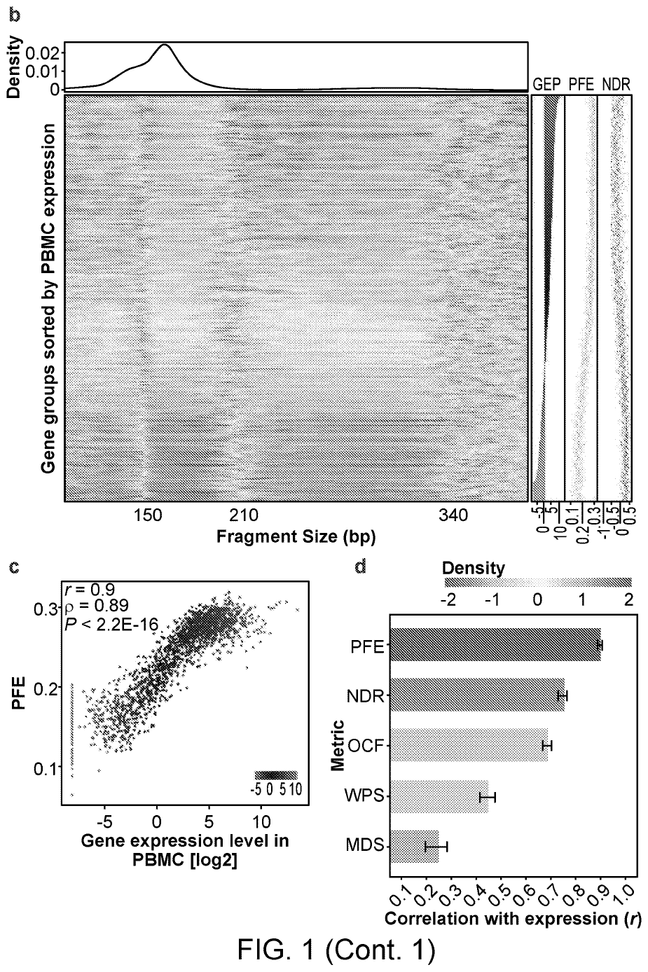

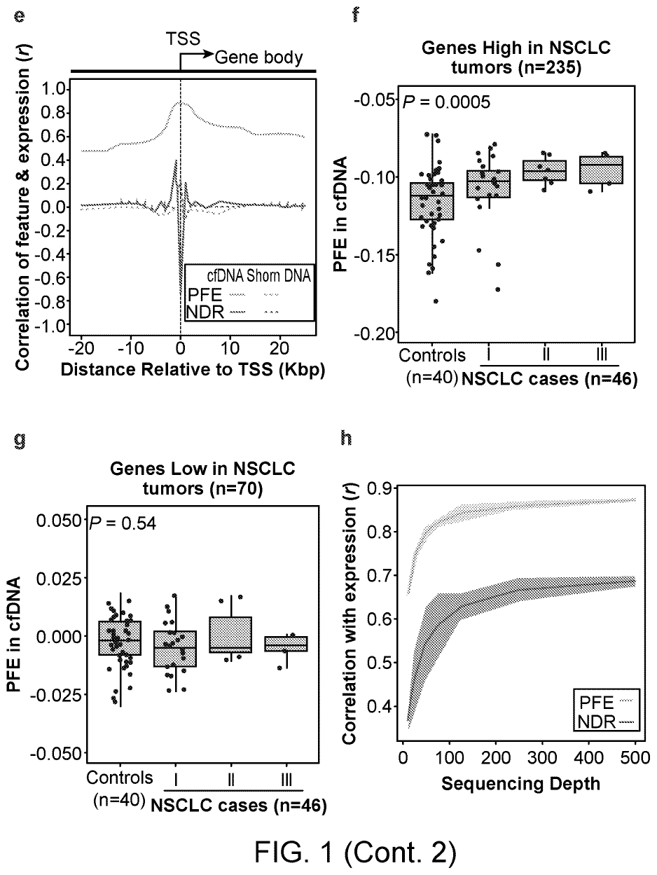

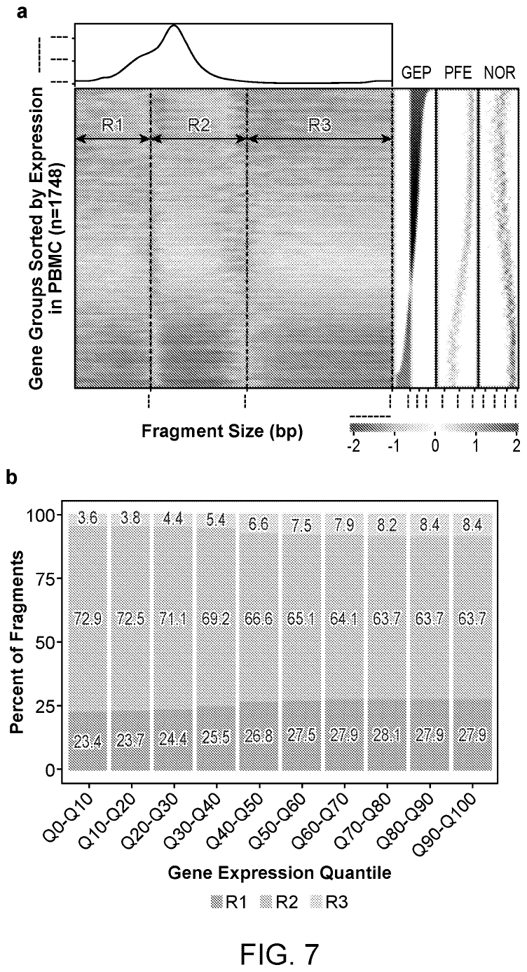

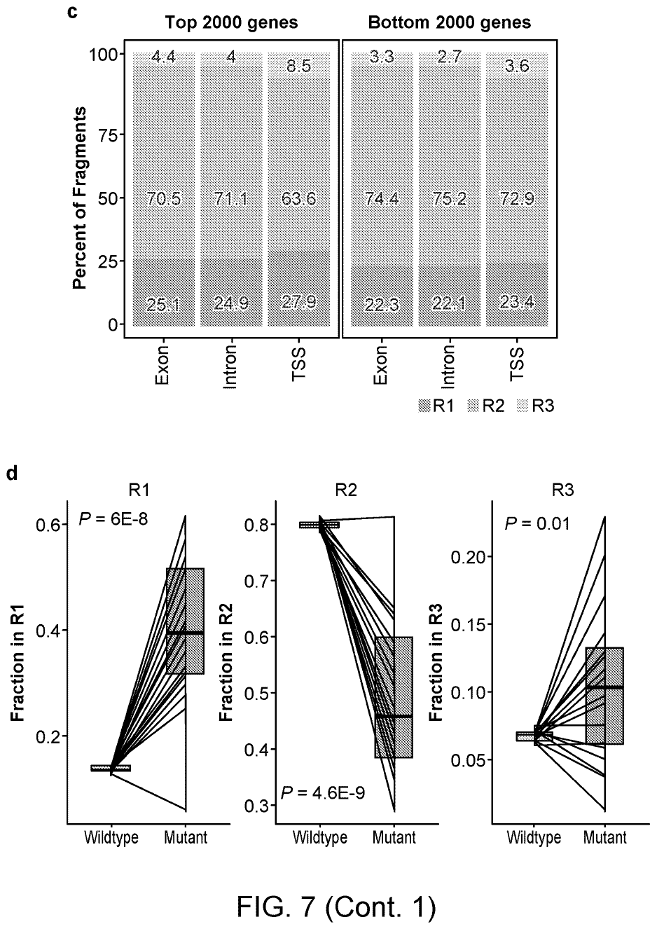

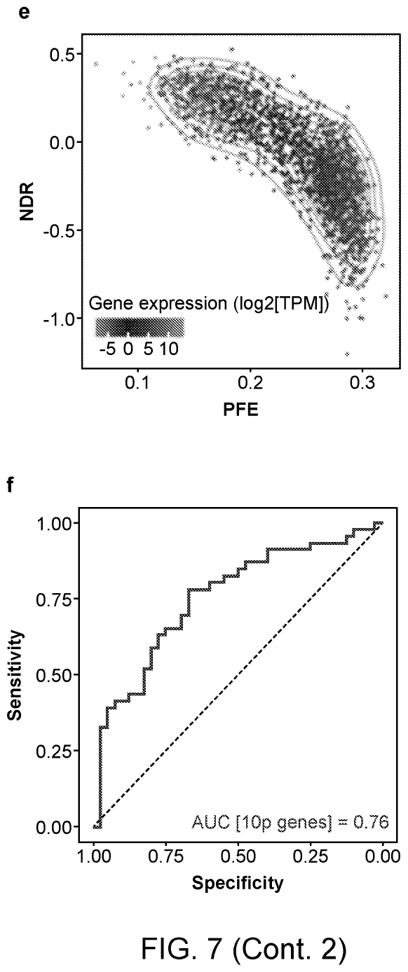

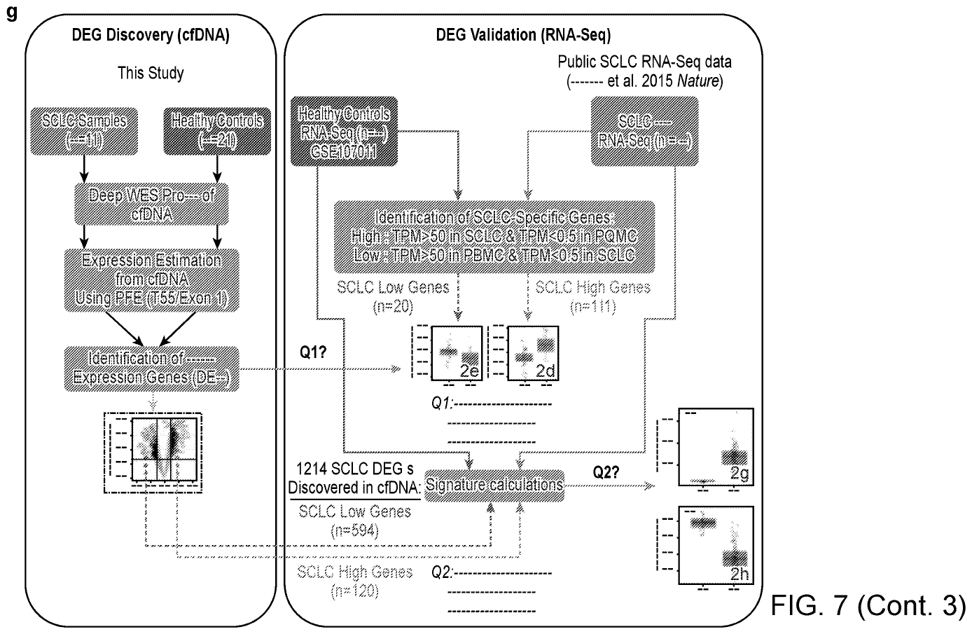

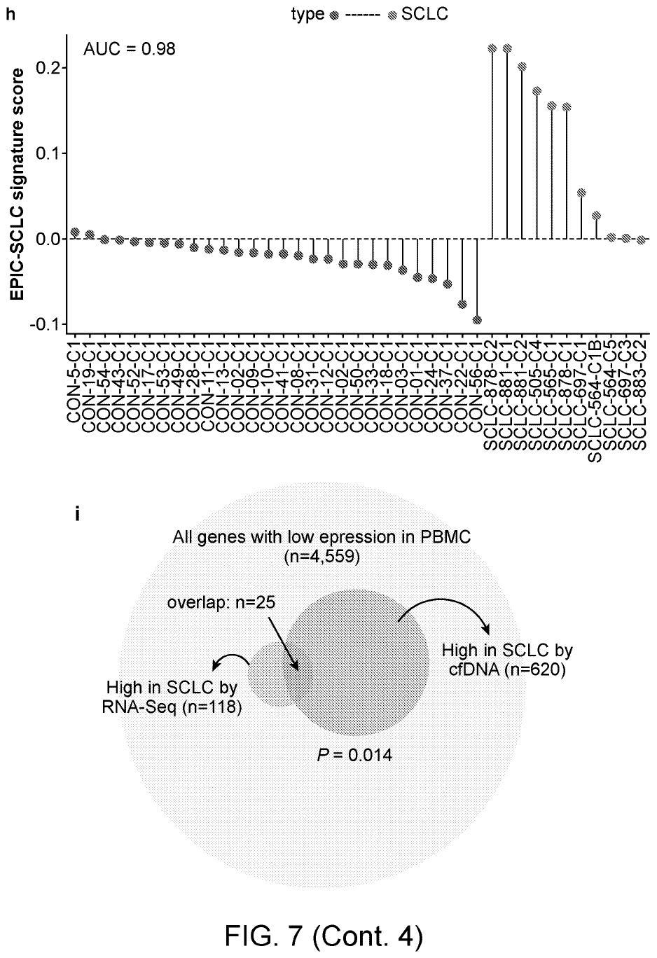

Cell-free DNA features correlated with gene expression. We hypothesized that cfDNA fragments from active promoters (which are less protected by nucleosomes) will exhibit more random cleavage patterns than fragments from inactive promoters (which are more protected by nucleosomes). If correct, this should allow inferences about the expression of individual genes from cfDNA, reflecting contributions from various cell types in diverse tissues, including solid tumors (FIG. 1a). To explore this hypothesis, we profiled cfDNA by relatively deep WGS (˜250×) from a patient with carcinoma of unknown primary (CUP) who had very low levels of ctDNA as quantified by personalized CAPP-Seq (<0.05%; Methods and Table 3) (Chabon, 2020). Since the vast majority of cfDNA molecules were therefore of hematopoietic origin (Moss, 2018), we correlated specific cfDNA fragmentomic features to expression levels of peripheral blood leukocytes determined by RNA-Seq. We then ranked genes by their expression levels and characterized the distribution of cfDNA fragments at their promoters (FIG. 1b). In support of our hypothesis, cfDNA molecules mapping to the ˜2 kb region flanking the TSSs of highly expressed genes exhibit substantially more fragment length diversity than fragments mapping to TSSs of poorly expressed genes. This phenomenon is especially prominent in sub-nucleosomal fragments (<150 bp and 210-300 bp. FIG. 1b and FIGS. 7a-b).

We reasoned that nucleosome displacement or depletion at the TSS of active genes could result in more diverse digested fragments iWeintraub, 1976), and that estimating this diversity could inform the corresponding expression level at individual gene TSS regions. We therefore captured this diversity in cfDNA fragment lengths as an entropy measure, calculating a modified Shannon index for fragments where both ends fell within the 2 kb flanking each gene's TSS (1 kb on each side). After adjusting this cfDNA entropy measure using a Dirichlet multinomial mixture (DMM) model for normalization, we refer to this metric as promoter fragmentation entropy (PFE; Methods). We observed remarkably high transcriptome-wide correlation between PFE measured in cfDNA by WGS and expression levels measured by RNA-Seq of peripheral blood mononuclear cells (PBMCs; R=0.89, P<1E-16; FIG. 1b-c, Table 4). While sequencing depth at the nucleosome-depleted regions flanking the TSS (NDR depth) (Ulz, 2016) was also significantly correlated with gene expression of corresponding genes, it showed substantially lower correlation than did PFE (FIG. 1b; r=−0.78. P<1E-16). The significant correlations between RNA expression levels and fragmentomic features were only observed in cfDNA and not in acoustically shorn high-molecular-weight genomic DNA from matched leukocytes (PFE r=0.003; NDR r=0.24). Accordingly, the expression inferences from cfDNA fragmentation profiles appear to reflect functional nucleosomal associations of DNA in vivo and are not predictable from the primary DNA sequence alone. Furthermore. TSS regions were distinguished from exonic and intronic by having the highest representation of subnucleosomal fragments (P<0.0001, FIG. 7c).

We also tested whether the partially protected, subnucleosomal cfDNA fragments that are 100-150 bases long could derive from tumor tissues. As previously described, in patients with non-small cell lung cancers (NSCLC) (Chabon, 2020), we observed cfDNA molecules harboring tumor mutations to have significantly higher representation of subnucleosomal fragments than their wild-type counterparts (P<6E-08, FIG. 7d). Therefore, the prevalence of subnucleosomal fragments observed in cfDNA correlate with expression levels and can derive from solid tumor origin.

We next compared several other cfDNA fragmentation features for correlation with gene expression levels of peripheral blood leukocytes (FIG. 1d, Table 4). While prior cfDNA profiling studies have reported lower depth of sequencing coverage at nucleosome depleted regions (NDR) within promoters of actively expressed genes (Ulz, 2016), the correlation between PFE and expression was stronger than the correlation between normalized NDR depth and expression (FIG. 1b,d). In addition to the advantages of PFE for expression inferences made from cfDNA profiles using NDR depth at TSS regions. PFE also outperformed other previously defined fragmentomic metrics including windowed protection score (WPS) (Snyder, 2016), motif diversity score (MDS) (Jiang, 2020), and orientation-aware cfDNA fragmentation (OCF) (Sun, 2019). We next examined whether the distance from the TSS impacts correlations between cfDNA fragmentomic features and gene expression. When considering the ˜20 kb region flanking each promoter, we observed the peak correlation between cfDNA PFE and gene expression to be centered at the TSS. However, in comparison to NDR, correlation of PFE with gene expression had broader dispersion and extended into regions flanking the TSS (FIG. 1e).

We further confirmed our observations from deep WGS profiling of cfDNA by considering fragmentomic profiles of lung cancer patients previously profiled at lower but more typical WGS depth (20×-40×) (Zviran, 2020). We compared lung cancer cases and healthy controls when inferring gene expression levels of two lung cancer gene signatures defined in primary tumor tissues, corresponding to genes highly or lowly expressed in non-small cell lung cancers (NSCLC). We observed a significant increase in the inferred expression levels of the NSCLC-high signature as distinguishing lung cancer from healthy non-cancer controls, associated with a monotonic relationship to lung cancer stage (FIG. 1f; Methods). Importantly, this increasing trend was not observed in the NSCLC-low expression signature (FIG. 1g), indicating the effect to be gene- and tissue-specific. Indeed, the NSCLC signature also showed modest performance in distinguishing lung cancer cases from controls when cfDNA was profiled by WGS (AUC: 0.76; FIG. 7f). We also investigated the impact of sequencing depth on correlations between cfDNA fragmentomic signals and transcriptome-wide RNA expression. Interestingly, correlations plateaued around ˜500× sequencing depth (FIG. 1h). Overall, these results indicated that cfDNA fragmentation features are strongly correlated with RNA expression, and that PFE better captures this correlation than previously described metrics studied.

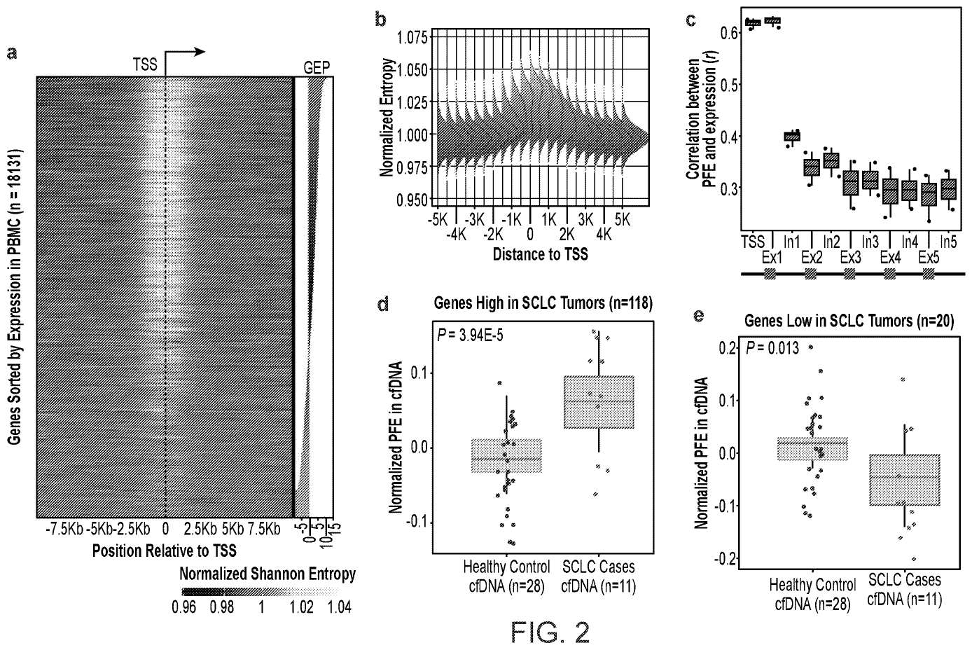

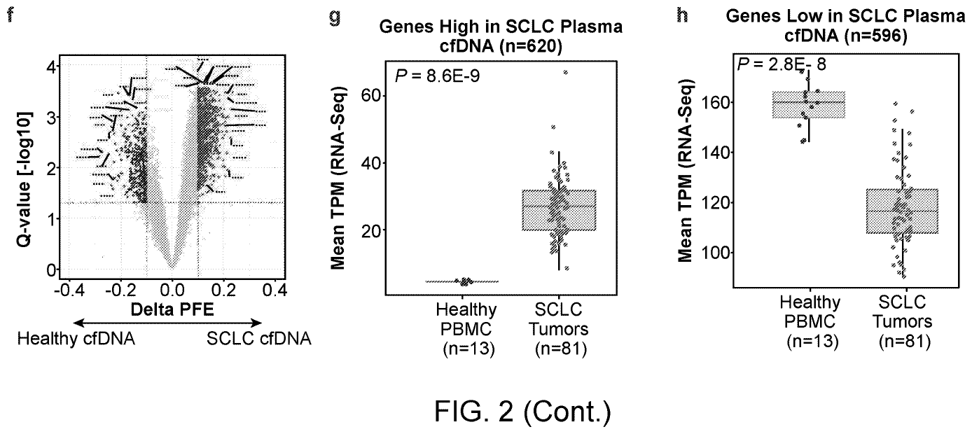

To better resolve the association between cfDNA fragmentation entropy and expression levels, we next studied their relationship across individual gene bodies, when considering distance from the TSS and exon/intron organization. We found peak cfDNA fragmentation entropy to be centered at the TSS, with this effect being most prominent for highly expressed genes (FIG. 2a). When summarizing results across genes as a function of distance from the TSS, we observed a bimodal distribution of entropy values in a ˜2.5 kb window flanking each TSS (FIG. 2b). When considering gene bodies, we found that while first exons display similar entropy signals as the TSS, this signal precipitously declines for subsequent introns and exons that are farther from the TSS (FIG. 2c). Therefore, cfDNA fragmentation features flaking TSS regions are highly correlated with gene expression levels across the transcriptome, with normalized entropy of cfDNA fragments overlapping first exons capturing much of this effect.

Validation of PFE expression inferences from cfDNA in solid tumors. Having observed that the fragmentation entropy of cfDNA molecules overlapping first exons correlates with gene expression inferences from WGS profiling, we next asked whether whole exome profiling (WES) could be used to validate inferred expression estimates from cfDNA. Specifically, we profiled plasma cfDNA of small cell lung cancer cases (SCLC, n=1l) and healthy controls (n=28) by ultradeep WES (median unique depth ˜2000×) to infer expression levels using PFE. We then compared these inferred results with expression levels observed in transcriptome profiling of solid tumor tissues by RNA-Seq (FIG. 7g). When considering genes known to be highly expressed in primary SCLC tumors as compared with PBMCs by RNA-Seq genes or vice versa (Methods), we found a striking concordance in the corresponding signatures in plasma cfDNA (FIG. 2d-e). Specifically. ‘SCLC high’ tumor genes had significantly higher normalized PFE levels in plasma cfDNA of SCLC patients than healthy controls (P=3.9E-5; FIG. 2d), and conversely, ‘SCLC low’ genes demonstrated the expected reciprocal pattern (P=0.02; FIG. 2e). When combining these two signatures into a single ‘SCLC score’ for each patient, we observed strong classification performance for distinguishing SCLC cases from controls (AUC=0.98, 95% CI: 0.94-1; FIG. 7h).

Separately, we asked whether the de novo discovery of SCLC-specific gene expression markers might be feasible noninvasively, when considering exome-wide cfDNA profiling and PFE overlapping first exons (FIG. 2f). Among such candidate differentially expressed genes that distinguished plasma cfDNA from SCLC cases versus healthy adult controls across the inferred transcriptome, we identified several well-known SCLC markers including ASCL1, ANKI, and ASTN1 (FIG. 2f). Indeed, genes whose differential expression was inferred from cfDNA exhibited highly significant and concordant differential expression in primary SCLC tumor tissues and PBMCs when profiled by RNA-Seq (FIG. 2g-h, Methods). Importantly. SCLC-specific genes inferred from plasma by WES profiling of cfDNA were highly enriched for genes observed to be highly expressed in primary SCLC tumors previously by RNA-Seq (P=0.014; FIG. 7i). Therefore, expression inference from cfDNA is feasible and can faithfully capture tumor-specific gene expression from solid lung cancer tissues at gene-level resolution.

Inferring Gene Expression from cfDNA Fragmentation Profiles

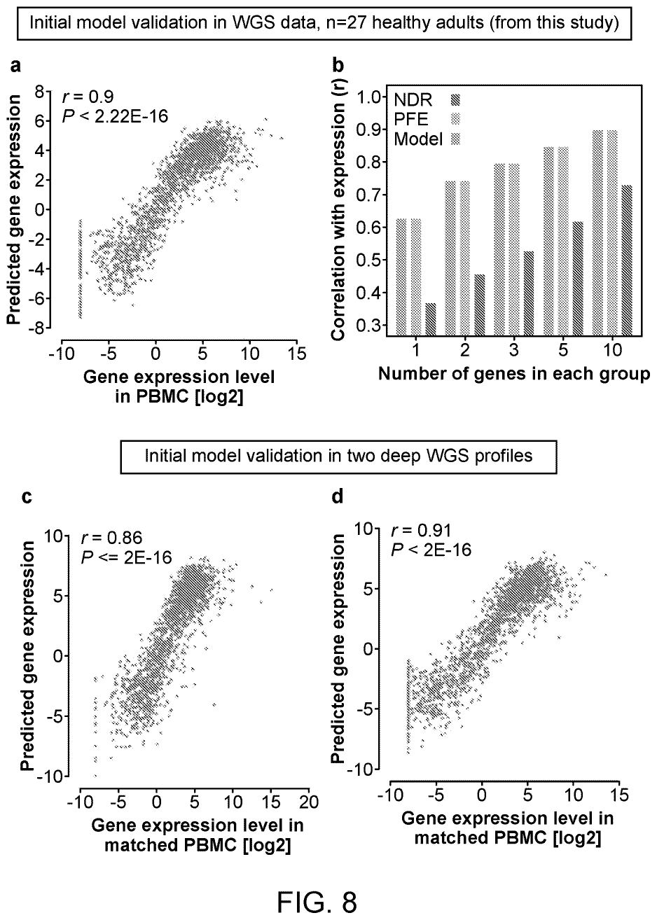

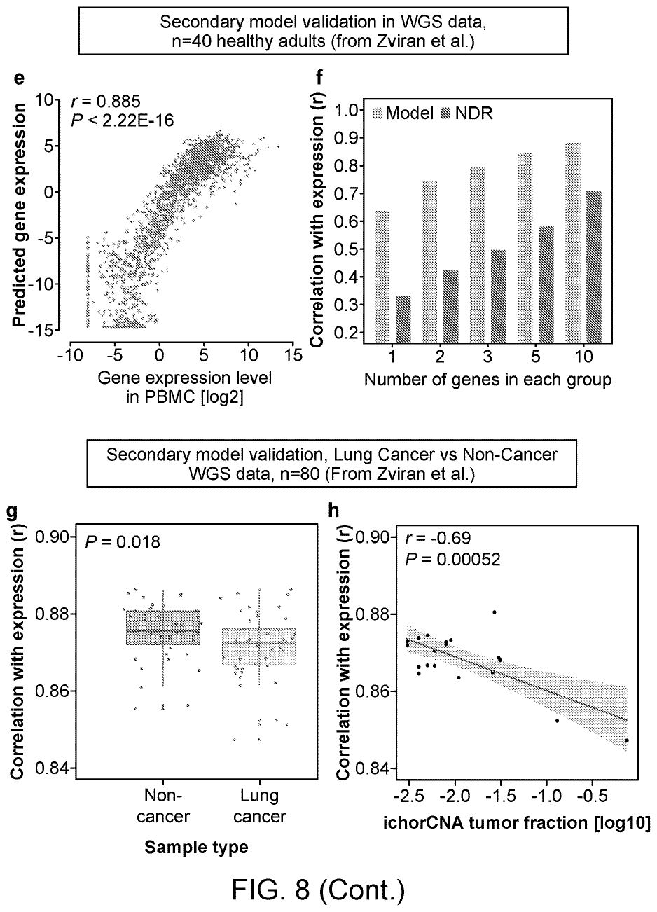

We next attempted to predict gene expression from cfDNA fragmentomic features derived by WGS. When considering diverse fragmentomic metrics, we identified PFE and normalized NDR depth as complementary features predicting RNA expression in an ensemble generalized linear model (Methods). Specifically, while cfDNA fragmentomic features were loosely correlated to each other, PFE demonstrated better dynamic range for lowly expressed genes, while highly expressed genes appeared better captured by normalized NDR depth (FIG. 7d). We then validated this ensemble model by applying it to a fragmentomic ‘meta-profile’ assembled by WGS profiling of plasma cfDNA from 27 healthy adults (Methods). Here again we observed high correlation between model-predicted expression levels and observed measurements by RNA-Seq of PBMCs when considering groups of 10 genes (r=0.9, FIG. 8a). Consistent with our prior observations (FIG. 1h), these correlations deteriorated at lower sequencing depth in a manner that hampered resolution at the level of single genes (r=0.9 for 10-gene bins versus 0.79 for 3-gene bins versus 0.64 for individual TSSs; FIGS. 8a-b). While cfDNA PFE outperformed NDR in correlations with expression, our composite model combining both PFE and NDR had marginal but consistently higher correlations than either alone (FIG. 8b).

We also examined the robustness of our gene expression inference model by considering its performance on cfDNA data from different subjects, and various independent ground truth transcriptome data sources obtained by RNA-Seq. We therefore profiled two additional cfDNA samples from two healthy adults by deep whole genome sequencing. As ground truth, we also profiled the matched leukocytes of these two individuals by RNA sequencing. In both cases, we found expression inferences from cfDNA WGS using our model to be strongly and significantly correlated across the transcriptome as measured by RNA-Seq TPM (r=0.86, and r=0.91 with P<2.2E-16, FIGS. 8c-d). Therefore, the generalized linear model described here appears robust for estimating gene expression levels from cfDNA and is not substantially impacted by the source of cfDNA, or by the ground-truth transcriptome data employed for training.

To validate the performance of our model in healthy controls versus patients with cancer, we next re-analyzed genome-wide cfDNA profiling data from 40 healthy adults and 46 patients with early-stage lung cancers that were previously profiled by WGS at ˜20-40× coverage¹⁵. We observed similar performance for predicting leukocyte gene expression levels when considering the average cfDNA meta-profile across the genome in the 40 healthy subjects (FIGS. 8e-f). When considering groups of 10 genes across the transcriptome. Pearson correlations between model predicted expression and expected RNA expression levels from PBMCs remained ˜0.85. Nevertheless, gene expression levels inferred from plasma cfDNA fragmentomic profiles of lung cancer patients were lower compared to PBMC transcriptomes (P=0.018; FIG. 8g). Hypothesizing that the lower correlation in lung cancer may be driven by an increased contribution of lung cancer-derived fragments, we used tumor fraction estimates by ichorCNA (Adalsteinsson, 2017) and observed a significant negative correlation with inferred leukocyte expression levels (r=−0.69, P=0.0005, FIG. 8h). This experiment demonstrates that tumor-derived cfDNA can substantially reduce the contribution of the leukocyte compartment to the cell-free nucleic acid pool, and this contribution can be measured by inferring tissue-specific gene expression from cfDNA when tumor burden is high.

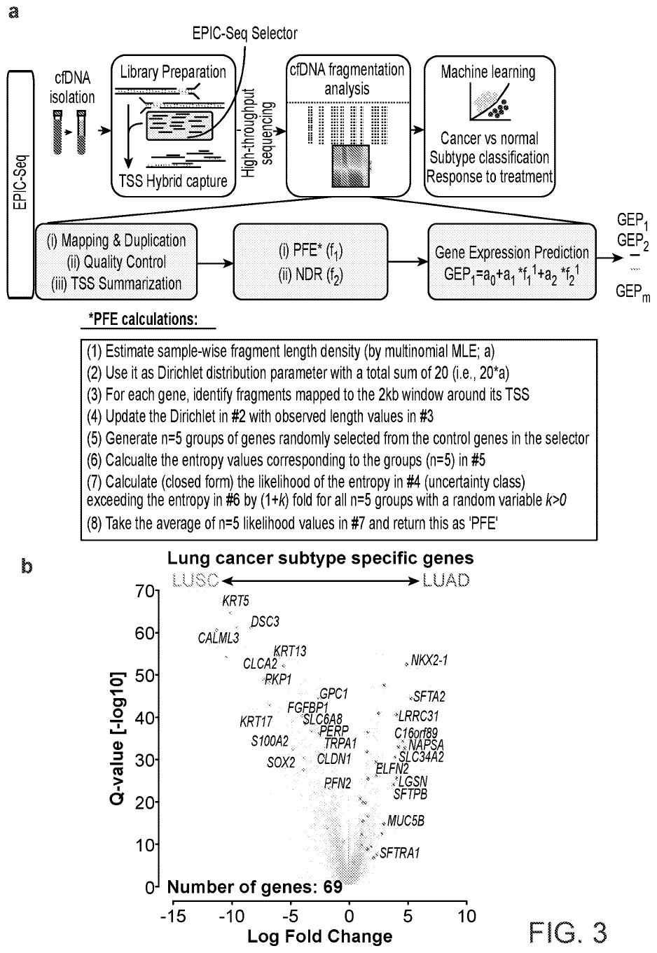

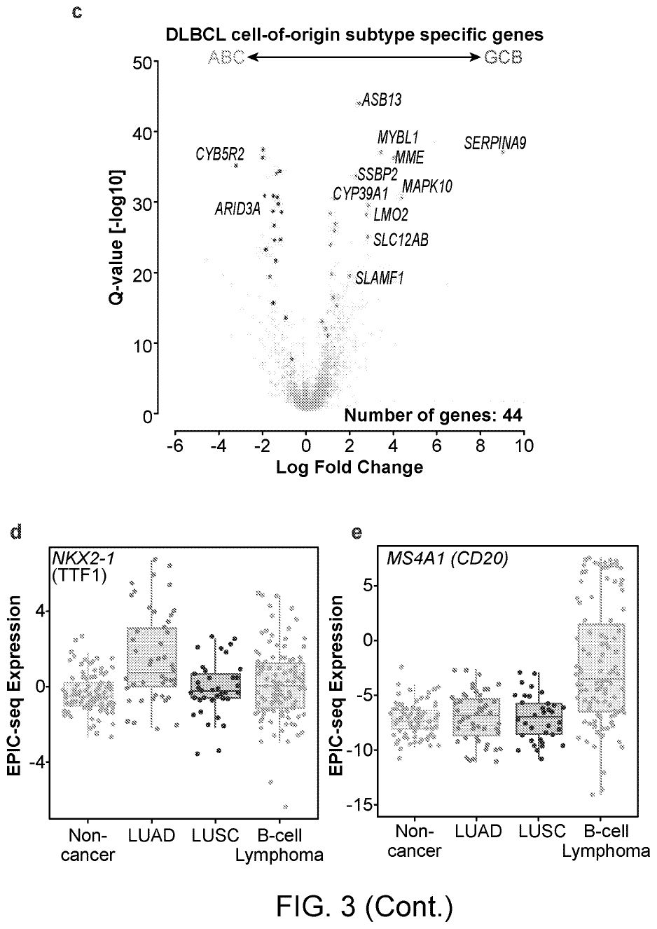

Epigenetic inference of expression by targeted deep cfDNA sequencing (EPIC-Seq). Based on our observation that PFE and NDR correlated better with gene expression at higher WGS sequencing depths (FIG. 1h), we next set out to develop a method allowing prediction of expression at the level of individual genes by deeper profiling of TSS regions. While normalized entropy of cfDNA fragments overlapping first exons could be used to infer expression levels when using deep WES (as described above for SCLC), the non-transcribed 5′ flanking regions of most genes are untiled by typical commercially available exome bait sets, thereby precluding corresponding NDR estimates from these TSS regions. Therefore, we devised a new approach—EPigenetic expression Inference from Cell-free DNA Sequencing (EPIC-Seq)—that combines hybrid capture-based targeted deep sequencing of TSS flanking regions in cfDNA with machine learning for predicting RNA expression (FIG. 3a). The TSS regions targeted in an EPIC-Seq experiment are tailored to include genes expected to be differentially expressed in the conditions of interest (e.g., cancer versus normal, histologic subtype A vs subtype B, etc.)

As a proof-of-concept, we tested this framework by applying EPIC-Seq to two cancer classification problems using cfDNA: 1) noninvasively distinguishing histological subtypes of the most common solid tumor (Non-Small Cell Lung Cancer [NSCLC]), and 2) resolving molecular subtypes of the most common hematological malignancy (Diffuse Large B-Cell Lymphoma [DLBCL]). For each of these malignancies, we first identified genes highly expressed in tumor tissues, but with relatively low expression in whole blood (Methods). We then identified subtype-specific genes by evaluating those differentially expressed in NSCLC adenocarcinoma (LUAD) versus squamous cell carcinoma (LUSC) and DLBCL germinal center B- (GCB) versus activated B-cell (ABC) like subtypes. Specifically, we identified 69 differentially expressed genes (DEGs) when stratifying 1.156 NSCLC tumors by histological subtype from The Cancer Genome Atlas (TCGA; n=601 LUAD (Cancer Genome Research, 2014) vs n=555 LUSC. (Cancer Genome Research, 2012), FIG. 3b, Table 5). We separately identified 44 DEGs when stratifying 381 DLBCL tumors by molecular cell-of-origin (COO) subtype from prior publications (n=138 GCB vs n=243 ABC tumors, FIG. 3c, Table 5) (Schmitz, 2018). In addition to these 113 genes for classification of lung cancers and lymphoma subtypes, we also included 50 genes that are differentially expressed in leukocyte subsets (Newman, 2015) as well as 16 genes as additional controls (Methods).

For each gene of interest, we designed probes to capture the ˜2 kb region flanking the TSS, then profiled plasma cfDNA from by deep sequencing of the targeted regions to a median ˜2,000× unique depth of coverage as previously described. (Chabon, 2016; Newman 2016).

In cfDNA fragmentomic profiles captured by WGS, we observed marginal gains in transcriptome wide correlations beyond ˜500× nominal coverage depth (FIG. 1h). Nevertheless, for our EPIC-Seq experiments and our modestly sized panel, we targeted ˜2000× unique depth (˜4-fold excess) for three reasons: (1) to guarantee saturation of the correlation plateau, (2) to avoid any gene-to-gene variability in accuracy of EPIC-Seq predictions of expression levels that might otherwise be attributable to spurious differences in depth variability due to non-uniform hybrid capture of the TSS regions of genes of interest, and (3) to address the lower partial concentration of cfDNA from non-hematopoietic tissues in circulation.

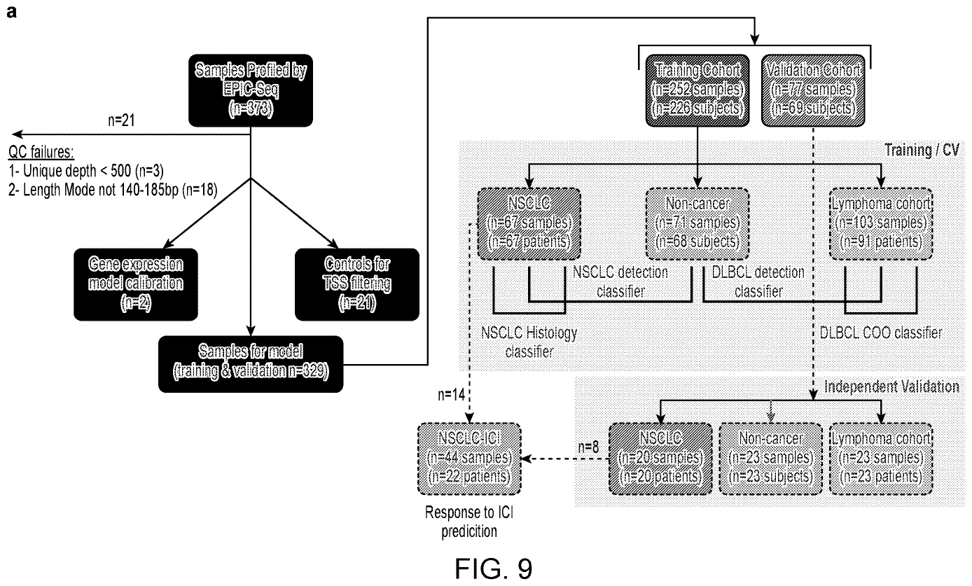

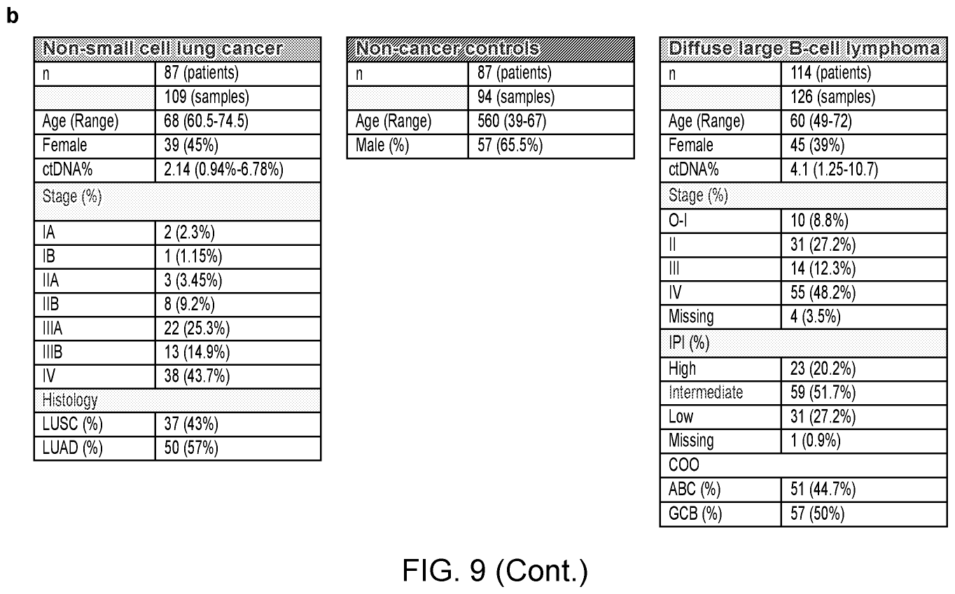

Using this workflow, we then profiled 373 plasma cfDNA samples, of which 329 were used for testing EPIC-Seq in different applications (FIG. 9a). This final set comprises 288 adults (FIG. 9a-b, Table 6), including 87 patients with NSCLC (n=109 samples), 114 patients with DLBCL (n=126 samples), and 87 otherwise healthy subjects (n=94 samples). Using a custom EPIC-Seq analytical pipeline (Methods), we computed cfDNA fragmentomic features for each gene of interest, and then estimated its predicted RNA expression level (FIG. 3a). To explore the ability of EPIC-Seq to infer the expression of individual genes, we next evaluated expression of NKX2-1 (TTF1), a gene highly expressed in LUAD and useful in histopathological diagnosis, and MS4A1 (CD20), a gene highly expressed in DLBCL and useful for immunophenotyping and classification of lymphomas (Maloney, 1994; Puglisi, 1999). Remarkably, the predicted expression level for NKX2-1 was significantly higher in plasma from patients with NSCLC-LUAD (Wilcoxon test P=4.2E-6; FIG. 3d). Conversely, the predicted expression level for MS4A1 was significantly higher in plasma from patients with DLBCL (Wilcoxon test P=4.2E-14; FIG. 3e). Collectively, these results illustrate that inference of expression is feasible by targeted deep cfDNA sequencing using EPIC-Seq, and that this framework can recover expected differences in tissue-derived expression at single-gene resolution.

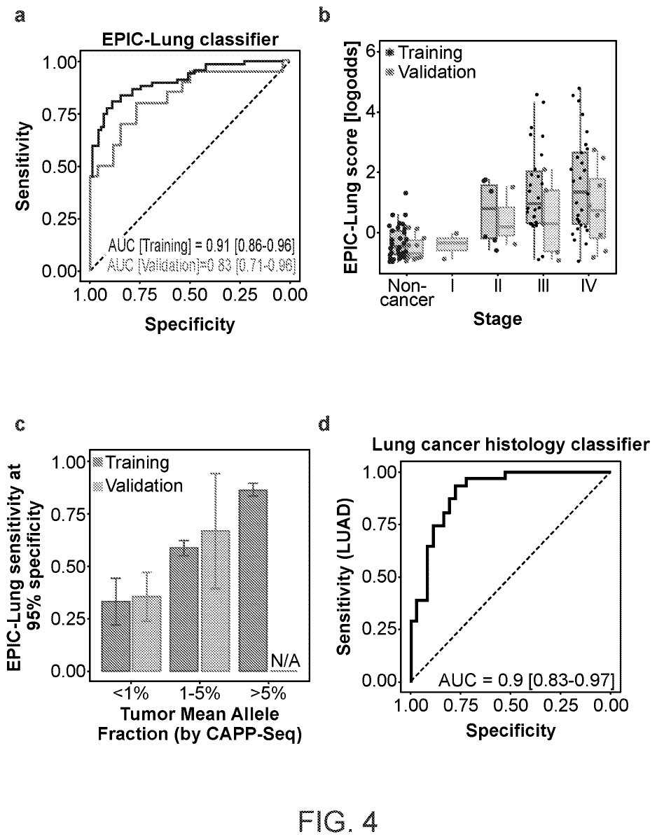

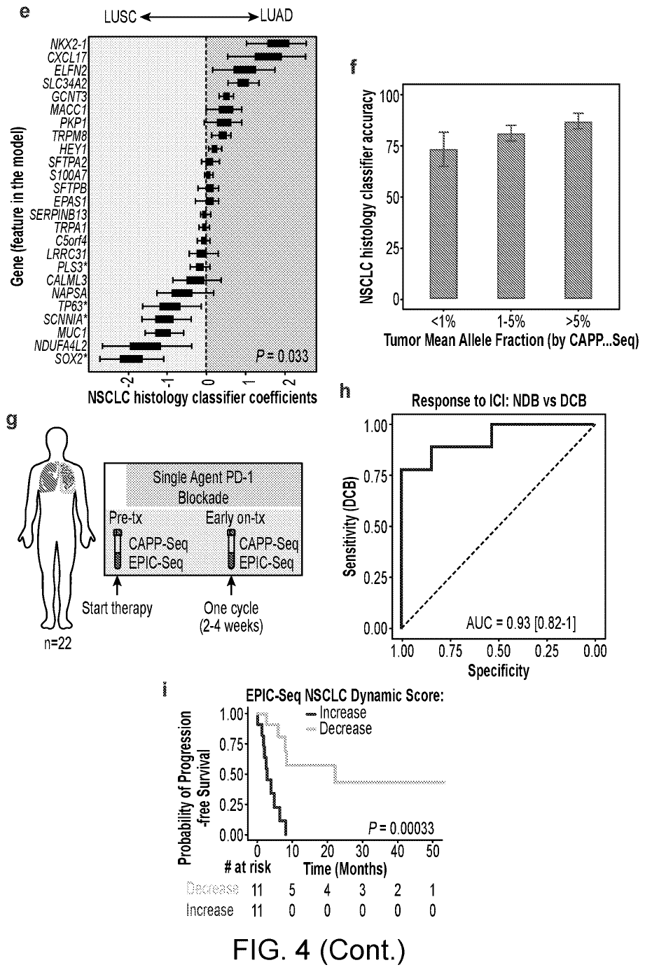

EPIC-Seq for lung cancer detection. We next evaluated whether EPIC-Seq might have utility for cancer classification problems, starting with lung cancer, the leading cause of cancer-related death in both men and women (Ferlay, 2014; Torre, 2016). We asked whether noninvasive classification of NSCLC cases versus healthy controls was feasible from cfDNA using EPIC-Seq. The cohort was split into training (n=138) and validation (n=43). A classifier trained on EPIC-Seq data to distinguish NSCLC patients (n=67, stage II (n=7), stage III (n=30) and stage IV (n=30)) from non-cancer controls (n=71) revealed robust performance (EPIC-Lung AUC=0.91, 95% CI: 0.86-0.96 based on leave-one-out cross validation) when considering 141 TSS sites from 117 genes (FIG. 4a; Methods). When we applied this trained classifier to the validation subset of NSCLC patients (n=20) and non-cancer controls (n=23), we again observed high classification accuracy, with only a modest decrease in performance (AUC=0.83, 95% CI: 0.71-0.96; FIG. 4a).

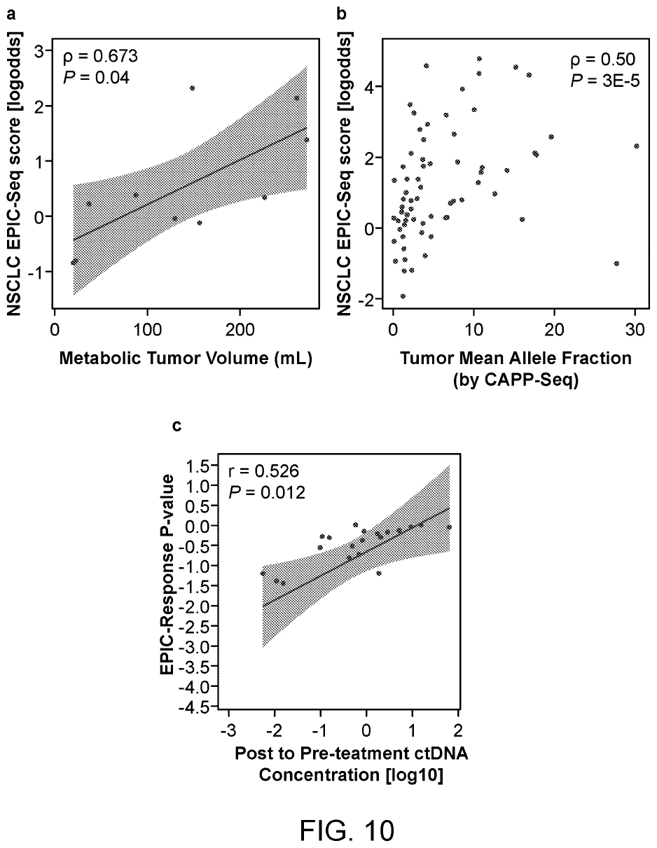

Epigenetic signals in cfDNA captured by our EPIC-Seq lung cancer classifier were significantly correlated with total metabolic tumor volumes (MTV), as measured by ¹⁸Fluorodeoxyglucose (FDG) uptake in combined positron emission tomography and computed tomography studies (PET/CT; ρ=0.67; P=0.04; FIG. 10a), consistent with higher ctDNA concentrations in patients with larger tumor burdens (Newman, 2014; Chabon, 2016). We also compared lung cancer epigenetic signals from EPIC-Seq in cfDNA with corresponding lung tumor-derived mutation signals from ctDNA separately measured by CAPP-Seq (Newman, 2015). Here again, EPIC-Seq lung signals in cfDNA seemed to capture tumor burden, as we observed significant correlation with the mean allelic fractions (AF) of tumor-derived somatic mutations measured by CAPP-Seq on the same specimens (ρ=0.5, P=3E-5; FIG. 10b). While most of the patients we profiled had advanced NSCLC, our classifier showed a statistical trend for stage III-IV cases having higher scores compared to stage II cases (P=0.08; FIG. 10b). We also assessed the importance of ctDNA concentration for the classifier's performance. When binning cases by ctDNA concentrations determined using mutations (CAPP-Seq), the EPIC-Seq lung classifier achieved ˜34% sensitivity at 95% specificity when allelic levels were below 1% and ˜86% sensitivity when ctDNA concentration exceeded 5% mean AF (FIG. 4c). Importantly, we observed similar sensitivity as a function of ctDNA fraction in the validation cohort (FIG. 4c). These results collectively demonstrate that RNA expression from lung tumors inferred by EPIC-seq can distinguish lung cancer cases from non-cancer individuals and correlate with tumor burden.

Noninvasive classification of NSCLC subtypes. Adenocarcinomas (LUAD) and squamous cell carcinomas (LUSC) represent the two most common histological subtypes of NSCLC (Travis, 2015) and differentiating between them can be an important step in determining the optimal treatment for patients (Reck, 2017; Ettinger, 2019). Currently the morphologic and immunophenotypic criteria used for this classification are determined using tissue specimens (Travis, 2015), but invasive evaluation can be fraught by diagnostic challenges and by procedural risks (Wiener, 2011; Bubendorf, 2017; McLean, 2018). Importantly, to the best of our knowledge, currently available mutation-based liquid biopsy methods are unable to reliably distinguish between LUAD and LUSC.

We therefore asked whether such classification could be performed non-invasively using EPIC-Seq. In a cohort of 67 NSCLC patients, a regression classifier for distinguishing histological subtypes (LUAD n=36; LUSC n=31) was trained on EPIC-Seq data and demonstrated robust performance in cross-validation studies (AUC=0.90, 95% CI: 0.83-0.97; FIG. 4d; Methods). The genes with largest coefficients and therefore strongest impact on the classification included canonical markers for LUAD (SLC34A2. NKX2-1 [TTF1]) and LUSC (SOX2), thus confirming biological plausibility of the classifier (Methods; FIG. 4e).

We evaluated the histology classifier's accuracy as a function of ctDNA levels as determined by CAPP-Seq (Methods) and as expected observed performance to be correlated with ctDNA concentration (FIG. 4f). Specifically, accuracy was highest at mean AFs above 5% (87%), with slight deterioration at levels between 1-5% (81%), and below 1% (73%) (FIG. 4f). These results demonstrate that inference of lung cancer expression differences by EPIC-seq allows for the noninvasive histological classification of NSCLC and that this framework appears robust across a range of ctDNA concentrations.

Predicting response to PD-(L)1 immune-checkpoint inhibition. For patients with advanced NSCLC, therapeutic blockade of programmed death 1 and programmed death-ligand 1 (PD-[L]1) signaling using monoclonal antibodies has shown remarkable promise (Reck, 2016; Socinski 2018). Trials combining PD-(L)1 blockade with cytotoxic therapy or with other immune checkpoint inhibition (ICI) strategies have demonstrated improved response rates at the risk of higher toxicity (Gandhi, 2018; Hellman, 2018). Since only a minority of NSCLC patients achieve durable benefit from ICI, there is a critical unmet need for reliable biomarkers that can accurately identify these patients before or early during ICI therapy. (Camidge, 2019).

We therefore performed an exploratory analysis to test the biological plausibility of tracking fragmentomic features as informative for therapeutic response monitoring. Specifically, we tested whether early, non-invasive assessment of response to PD-(L)1 immune-checkpoint inhibitors might be feasible using EPIC-Seq. To do so, we analyzed 22 longitudinal blood specimens from 22 NSCLC patients treated with PD-(L)1 blockade using EPIC-Seq. Samples were collected immediately before PD-(L)1 therapy and within the first four weeks of therapy initiation (FIG. 4g). We developed a ‘lung dynamics index’ from EPIC-Seq predicted gene expression as a function of therapeutic benefit from ICI (Methods). This index demonstrated a significant correlation to mutation-based response assessment using CAPP-Seq on the same specimens (r=0.526, P=0.012, FIG. 10c) (Nabet, 2020). Importantly, this epigenetic metric was also able to distinguish patients achieving durable clinical benefit (DCB; defined as no progression for at least 6 months after start of therapy) from those with no durable clinical benefit (NDB) achieving an AUC of 0.93, 95% CI: 0.78-1 (FIG. 4h). Moreover, when stratified by the median index score in Kaplan-Meier analysis, patients with higher scores had significantly better outcomes (log-rank P=0.0003, FIG. 4i). Of note, within the limitations of this small cohort, we also observed a significant and continuous association of EPIC-Seq classifier scores with progression-free survival (HR=11.38; Wald P=0.006). Therefore, this proof-of-concept suggests that EPIC-Seq can reliably detect tissue-specific signals in NSCLC and can faithfully monitor response to ICI in predicting durability of associated clinical benefit.

Noninvasive DLBCL quantitation using EPIC-Seq. Diffuse large B cell lymphoma (DLBCL) is the most common Non-Hodgkin's lymphoma (NHL) and displays remarkable clinical and biological heterogeneity (Menon, 2012). While aspects of this heterogeneity can be captured by clinical risk indices such as the International Prognostic Index (Sehn, 2007), gene expression profiling (Alizadeh, 2000), or genotyping of primary tumor biopsies (Pasqualucci, 2011), it remains unclear whether such stratification might also be feasible using less invasive approaches.

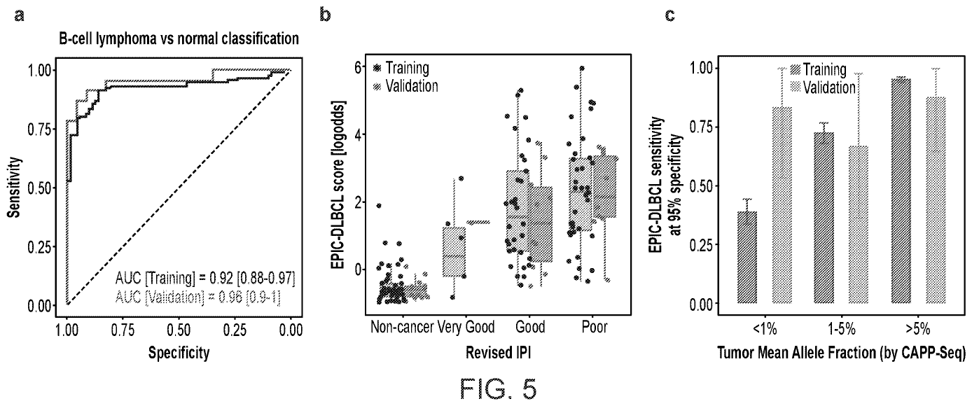

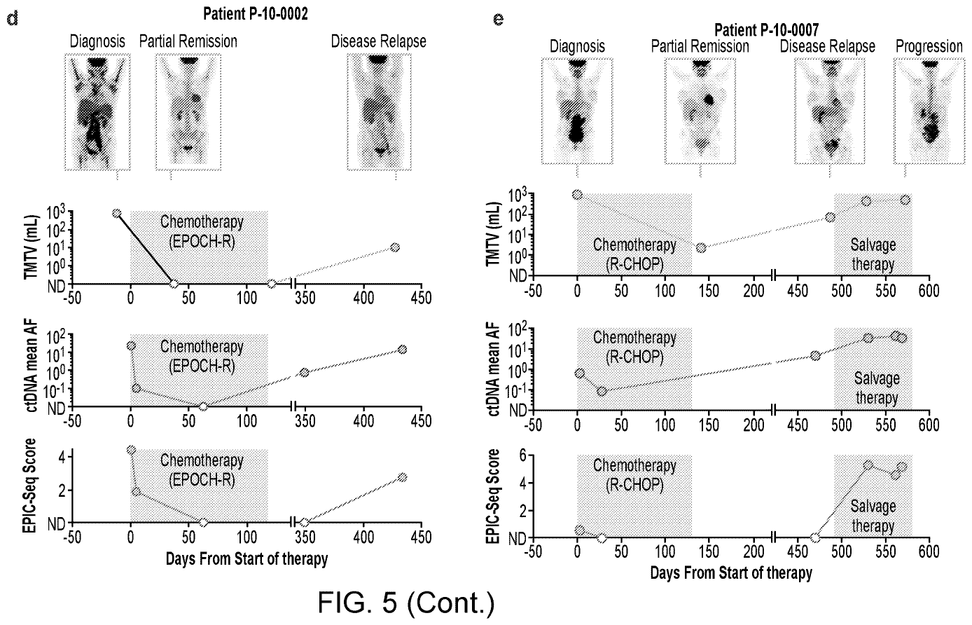

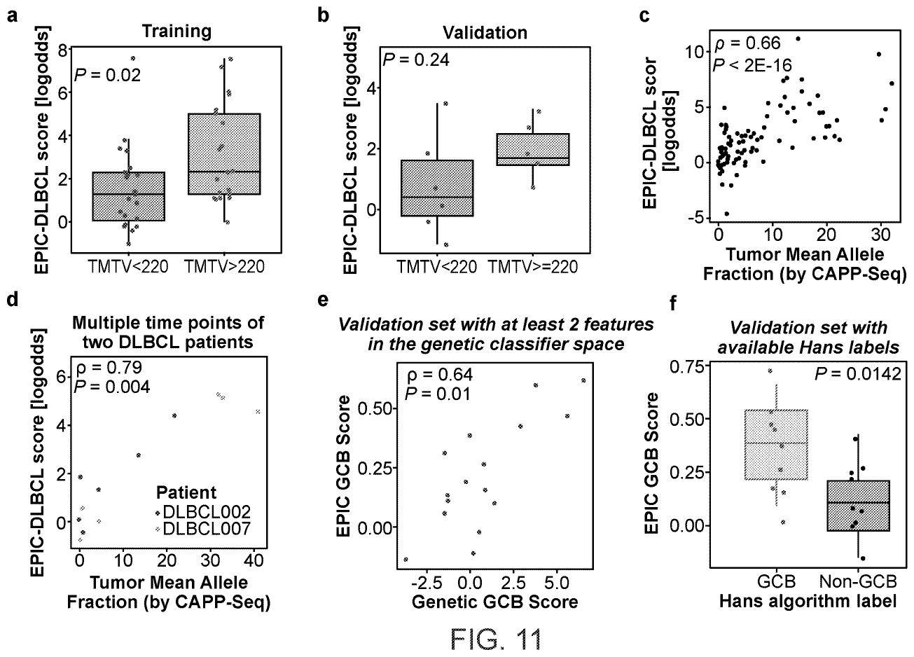

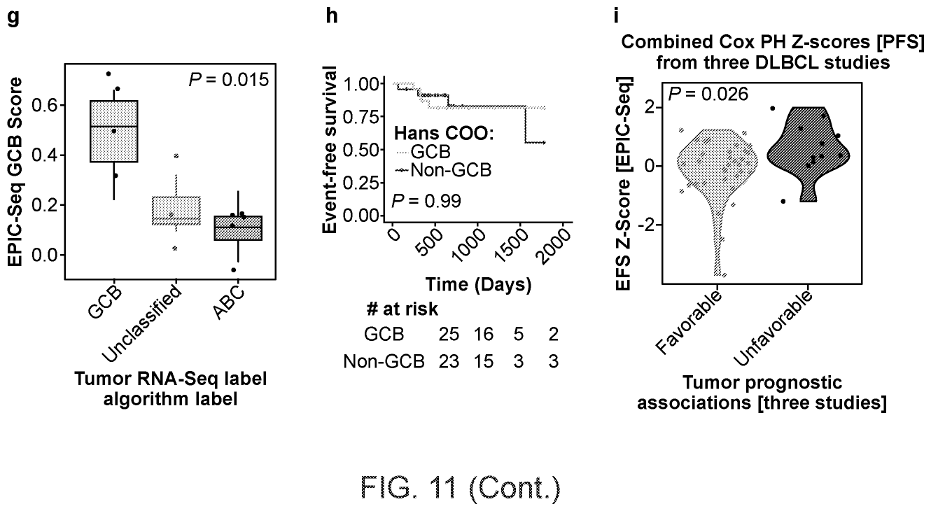

We therefore analyzed pre-treatment blood samples from DLBCL patients using EPIC-Seq and tested whether epigenetic signals in cfDNA allow noninvasive detection of DLBCL cases, distinguishing cancer patients from healthy controls. Here again, a regression classifier trained on EPIC-Seq data to distinguish DLBCL patients (n=91) from non-cancer controls (n=71) revealed robust performance (EPIC-DLBCL AUC=0.92, 95% CI 0.88-0.97 from leave-one-out cross validation; FIG. 5a; Methods). When we applied this trained classifier to a validation cohort of DLBCL patients (n=23) and non-cancer controls (n=23), we observed similar performance in distinguishing cancer from non-cancer (AUC=0.96, 95% CI 0.9-1; FIG. 5a). We also observed a significant graded relationship between scores from this epigenetic classifier and the Revised International Prognostic Index (R-IPI: Jonckheere's trend test P=0.004; FIG. 5b). Separately, for patients with available PET/CT scans, we also observed a significant trend for scores from the epigenetic classifier in distinguishing patients with high versus low tumor burden (Cottereau, 2016) as measured by total MTV (Wilcoxon P=0.015; FIG. 11a). This same trend was also observed in the validation set (FIG. 11b).

To further evaluate how EPIC-Seq scores reflect tumor burden in cfDNA, we compared them with the mean allele fractions (AFs) of mutations previously measured by CAPP-Seq on the same blood specimens (Scherer, 2016; Kurtz, 2018). Notably. DLBCL epigenetic scores determined by EPIC-Seq were strongly correlated with the mean mutant AFs determined by CAPP-Seq (ρ=0.66, P<2E-16; FIG. 11c). We also evaluated the performance of our classifier at various ctDNA levels. Specifically, when trying to distinguish lymphoma cases from non-lymphoma subjects as controls and considering various mean AF thresholds determined by CAPP-Seq, we calculated the sensitivity for DLBCL detection at 95% specificity. While EPIC-Seq's sensitivity was strongly related to mean AF and showed most robust performance at ctDNA levels above 1%, we observed ˜40% detection of DLBCL cases where mean AF was below 1% before therapy (FIG. 5c).

To assess the relationship between epigenetic signals and somatic mutations during DLBCL therapy and their stability over time, we next profiled serial blood samples from 2 patients shortly after induction therapy with curative intent using both EPIC-Seq and CAPP-Seq (n=12; FIG. 5d-e). Again, we observed strong and significant correlations between DLBCL EPIC-Seq scores and ctDNA concentrations over time in both patients (ρ=0.79, P=0.004, FIG. 11d), despite the administration of combined chemoimmunotherapy and the substantial attendant changes in leukocyte blood counts. Collectively, these results illustrate that expression inferences by EPIC-seq can noninvasively detect tissue-derived DLBCL signals and faithfully reflect disease burden before and after DLBCL therapy.

DLBCL cell-of-origin classification. Most DLBCL tumors can be classified into two transcriptionally distinct molecular subtypes, each derived from a specific B cell differentiation state (cell of origin [COO]): germinal center B cell-like (GCB) and activated B cell-like (ABC) (Alizadeh, 2000; Rosenwald, 2002; Basso, 2002). These subtypes are prognostic with significantly better outcomes observed in patients with GCB tumors, and may also predict sensitivity to emerging targeted therapies (Dunleavy, 2009; Thieblemont, 2011; Scott, 2014; Nowakowski, 2015; Wilson, 2015; Young, 2013). While this classification of DLBCL is among the strongest prognostic factors and a potential biomarker for personalized therapies, accurate subtyping remains challenging in clinical settings (Zelentz, 2019).

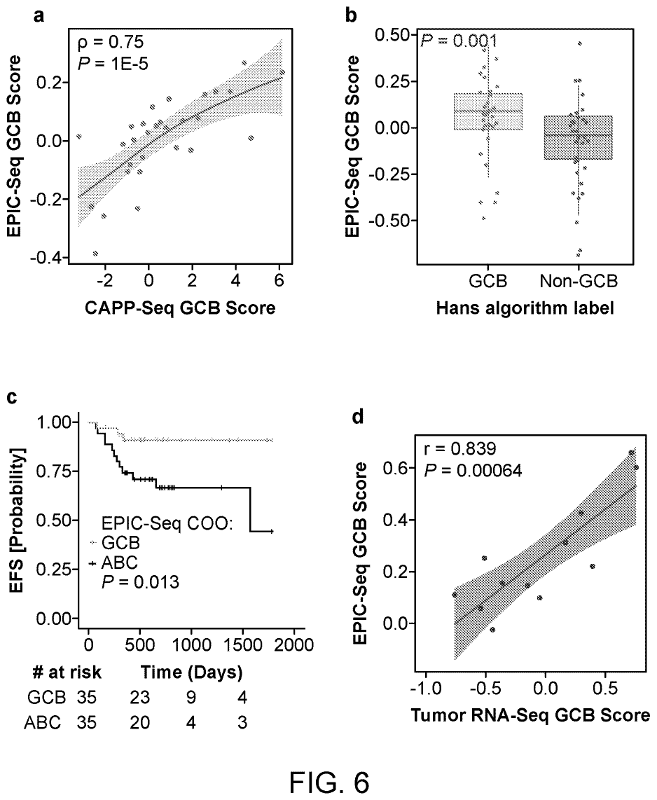

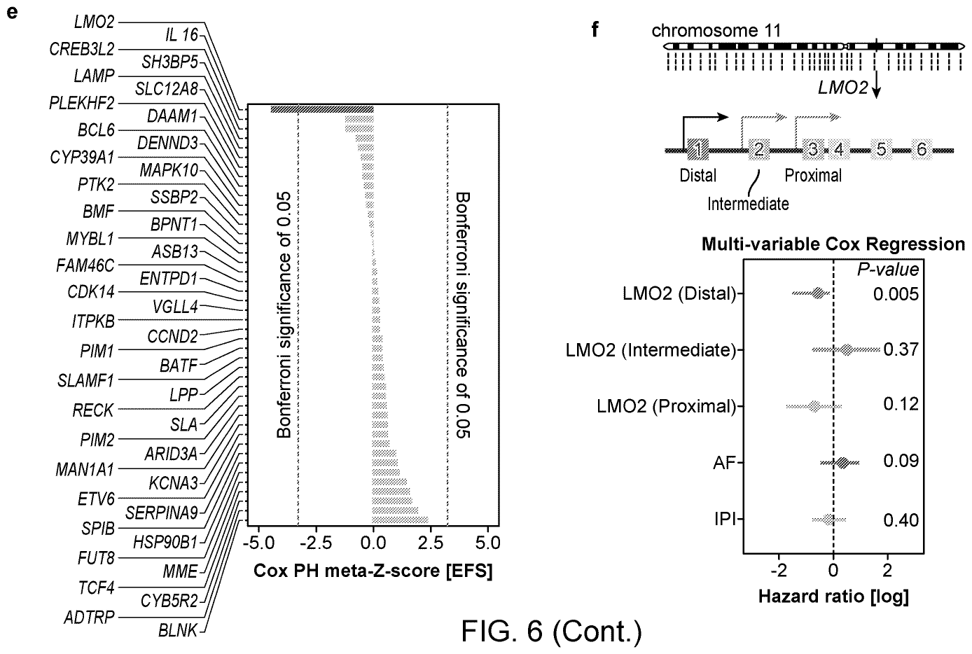

We therefore used EPIC-Seq profiling to develop a noninvasive COO classifier from pretreatment plasma. By considering differentially expressed genes in GCB or non-GCB (ABC) DLBCL and targeted by our panel, we built a probabilistic COO classifier analogous to those described above (Methods). When we benchmarked this classifier's performance in our cohort of 91 DLBCL patients, we observed epigenetic scores to be significantly correlated with previously described mutation-based GCB scores (ρ=0.75, P=1E-5, FIG. 6a) (Scherer, 2016). When we examined this epigenetic COO classifier in the validation set, we observed a significant correlation between EPIC-Seq scores and the mutation-based GCB scores (ρ=0.64, P=0.01, FIG. 11e). When comparing patients classified by the more commonly clinically used immunohistochemical Hans classification algorithm (Hans, 2004), we observed a significantly higher COO score for GCB cases compared with Non-GCB (Training: n=66. Wilcoxon P=0.001, FIG. 6b; Validation: n=18. P=0.014, FIG. 11f).

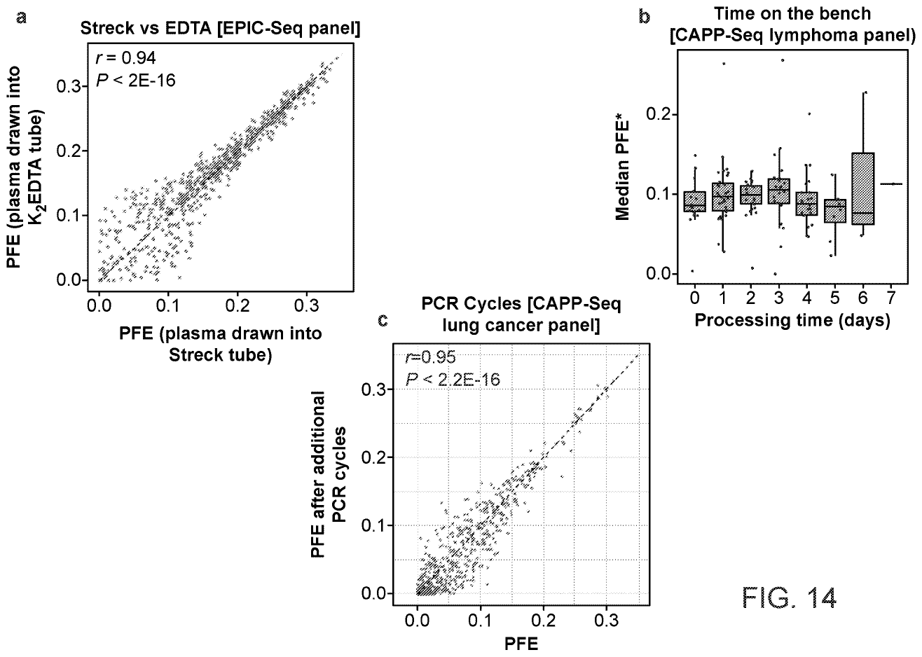

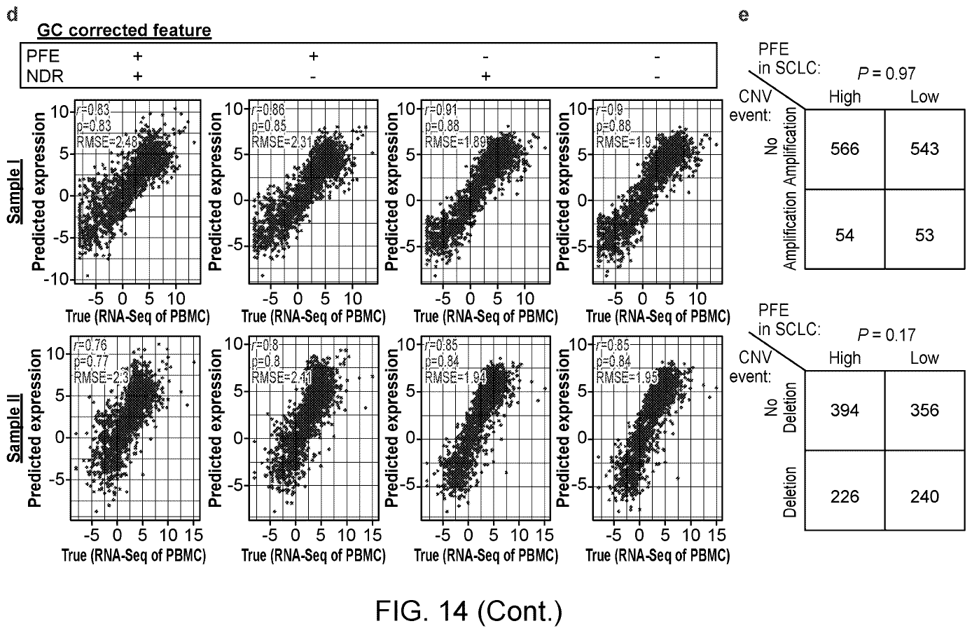

Comparing the expected prognostic power of epigenetic and mutation-based COO scores using univariate Cox regressions, we observed a stronger association between EPIC-Seq GCB scores and favorable outcomes in the frontline therapy cases (n=70, EPIC-Seq: HR=0.13, P=0.033 vs CAPP-Seq: HR=0.95, P=0.62). Indeed, when stratified by the median GCB score in a Kaplan-Meier analysis, patients with higher GCB scores had significantly better outcomes (log-rank P=0.013, FIG. 6c). Among patients analyzed by both immunohistochemistry and DNA genotyping, the Hans algorithm failed to stratify patient clinical outcomes, suggesting more accurate classification by our approach (FIG. 11h). To further characterize the fidelity of our plasma cfDNA classification results, we next expression profiled tumor biopsies of a subset of our DLBCL validation cases (n=12) by RNA sequencing. When assessing the concordance between EPIC-seq scores obtained from plasma cfDNA and COO scores obtained from tumor tissues by RNA-Seq, we found a significantly high correlation between these two orthogonal approaches (r=0.84, FIG. 6d; FIG. 14g). Overall, these results suggest that EPIC-Seq has utility for noninvasive classification of DLBCL cell-of-origin and can stratify patients better than both the genetic COO classifier and the Hans algorithm.

Determining prognostic power of individual genes with EPIC-Seq. Expression profiling studies for a variety of tumor types have identified the prognostic power of individual genes for both risk stratification and therapeutic management. In DLBCL, prior studies have validated the prognostic utility of several key genes in relatively large patient populations that were homogenously treated with modern combination immune-chemotherapy using R-CHOP (Lossos, 2004; Malumbres, 2008; Alizadeh, 2009; Alizadeh, 2011). These studies have relied on expression profiling from tumor biopsy specimens, which can be hampered by limitations of RNA sample quality and quantity.

Therefore, we wished to evaluate the utility of EPIC-Seq for noninvasively measuring expression of genes with prognostic associations in DLBCL. Using univariate Cox proportional hazard regression models, we tested the prognostic value of individual genes using pre-treatment blood plasma from 69 patients and used Z-scores to measure the relative strength of these associations. We first assessed the prognostic concordance of our results in blood plasma against primary tumor specimens by examining the correlation between our EPIC-Seq results with those described in 3 recent tumor expression profiling studies that relied on surgical DLBCL tissue specimens (Schmitz, 2018; Chapuy, 2018; Ennishi, 2019). When comparing the prognostic value of genes profiled in this manner, we observed a significant correlation of Z-scores from our study using plasma cfDNA with prior studies using tumor RNA (P=0.026; FIG. 11i).

Within our cohort, only LMO2 emerged as significantly associated with progression-free survival after correction for multiple hypothesis testing (nominal P=7.5E-6, corrected P=0.0055; FIG. 6e). This is consistent with prior data on its robust prognostic effect in DLBCL (Gentles, 2001). LMO2 is an oncogene consisting of six exons, of which three nearest the 3′ end are protein coding (Chambers, 2015). Inclusion of the three noncoding 5′ LMO2 exons is governed by alternative proximal (Royer-Pokora, 1995), intermediate (Oram, 2010), and distal promoters (Boehm, 1990). When comparing predicted expression from each of these alternative promoters for prognostic strength in DLBCL using EPIC-Seq, only the distal TSS (GRCh37/hg19-chr11:33.913.836) showed a significant association with outcome (FIG. 6f). Higher predicted expression from the distal TSS of LMO2 remained prognostic of more favorable outcomes in multivariable Cox regression after adjusting for IPI and ctDNA level (FIG. 6f). This result is consistent with the known importance of the distal LMO2 promoter in driving expression of LMO2 in human tumors, as evidenced by retroviral insertional mutagenic events observed in human gene therapy trials and chromosomal rearrangements mediating lymphomagenesis (Chambers, 2015). Collectively, these observations indicate that EPIC-Seq has utility for noninvasively measuring the expression and prognostic value of individual genes and for resolving their individual TSS regions.

Bait Set for Detecting Lymphomas and Identifying Subtypes Thereof. A bait set for enrichment of cell-free DNA molecules in proximity to transcription start sites of genes useful in detecting lymphomas and identifying subsets thereof was generated. Specifically, the transcription start sites for ˜1600 genes were identified (Table 1). A panel of selectors (i.e., a bait set) was developed that was designed to enrich from cell-free DNA that originated from regions within 750 bp (both upstream and downstream) of these transcription start sites. Stated differently, the bait set included biotin-tagged nucleic acid probes that were 93 or more bases in length for enriching cell-free DNA from regions within 750 base pairs of each of the transcription start sites identified in Table 1. In some cases, multiple probes were used to interrogate each 1.5 kb region spanning each transcription start site.

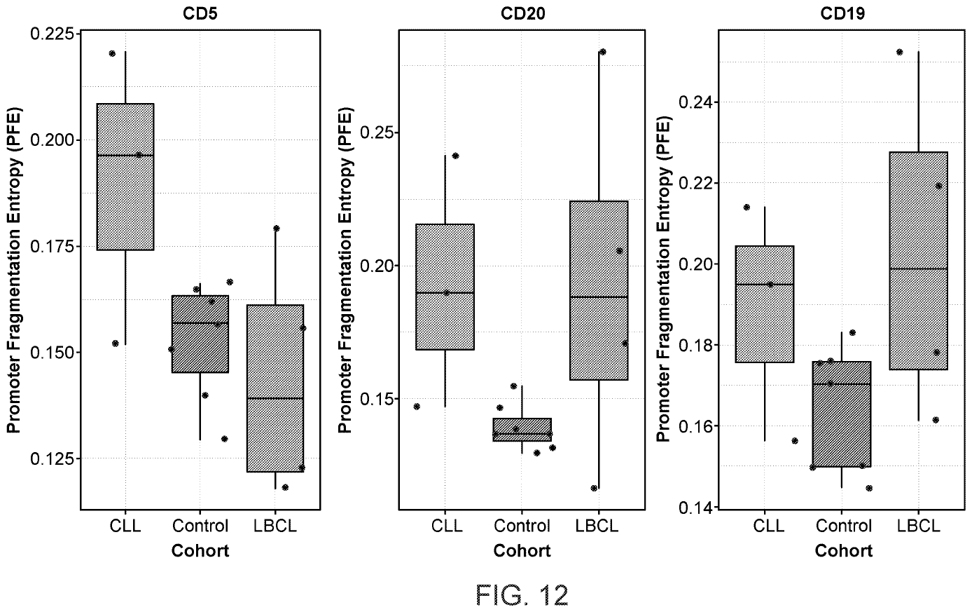

The bait set was used to enrich cell-free DNA samples. More specifically, the bait set was used to perform EPIC-seq profiling of plasma cell-free DNA from healthy controls (n=7), patients with chronic lymphocytic leukemia (n=3), and patients with diffuse large B-cell lymphoma (n=3). An exemplary analysis focused on three genes: CD5, CD20 and CD19. As expected, CD5 PFE levels are higher in the CLL cases (FIG. 12). The PFE levels of CD19 and CD20 are also, as expected, higher in the DLBCL cases (FIG. 12).

The bait set can be useful in identifying lymphomas and subtypes thereof, such as diffuse large B-cell lymphoma, chronic lymphocytic leukemia, Hodgkin lymphoma, follicular lymphoma, transformed follicular lymphoma, and mantle cell lymphoma. In some embodiments, the bait set further includes probes for enriching housekeeping genes, such as any subset of gene reported at https://www.tau.ac.il/˜elieis/HKG/ can be used a positive controls (having large PFE levels due to high expression across various cell types). In some embodiments, the bait set can further include probes that are designed to enrich for regions of the genome that are not expressed under typical conditions or are not adjacent to transcription start sites as negative controls.

Bait Set for Immune Response. A bait set for enrichment of cell-free DNA molecules in proximity to transcription start sites of genes useful evaluating immune responses (e.g., identifying responders to checkpoint inhibitor therapies) was generated. The genes identified in Table 2 include the following: (1) genes involved in the CD8 T cell exhaustion lineage, (2) primary regulators of exhausted T cells (TOX), (3) genes differentially regulated in a subset of CD8 T cells preferentially re-invigorated by ICI (Ki67), (4) genes related to response to ICI (T cell-inflamed gene expression profile, IFNG.GS, ISG.RS), (5) genes in tissue resident T/B cells, (6) genes differentially regulated in CD8+ and CD4+ neoantigen-reactive TILs, (7) genes differentially regulated in B cell maturation & activation, (8) marker genes of plasma cells, and (9) LM22 genes.

Specifically, the transcription start sites for ˜1050 genes were identified (Table 2). A panel of selectors (i.e., a bait set) was developed that was designed to enrich from cell-free DNA that originated from regions within 750 bp (both upstream and downstream) of these transcription start sites. Stated differently, the bait set included biotin-tagged nucleic acid probes that were that were 120 or more bases in length for enriching cell-free DNA from regions within 750 base pairs of each of the transcription start sites identified in Table 2. In some cases, multiple probes were used to interrogate each 1.5 kb region spanning each transcription start site. The bait set can be designed to interrogate between 1.5 and 2.5 MB of the human genome.

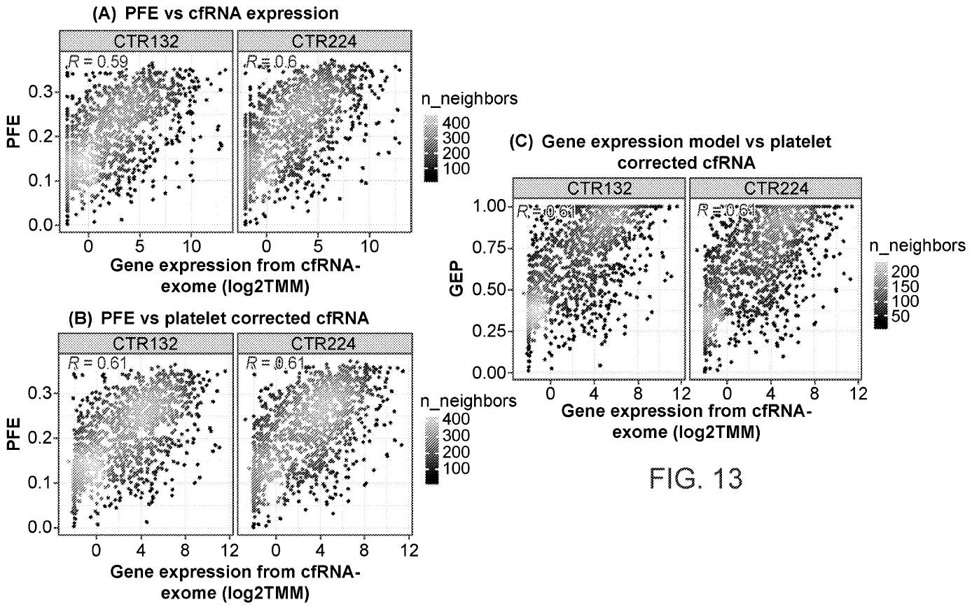

The bait set was used to enrich cell-free DNA samples. More specifically, the bait set was used to perform EPIC-seq profiling of plasma cell-free DNA from three healthy individuals. Cell-free RNA sequencing was also performed on matched time points of the same individuals. The PFE values calculated using the EPIC-seq pipeline were then compared with the RNA expression levels from cfRNA. A significant correlation was observed between PFE (calculated via DNA) and cfRNA expression (FIG. 13).

The bait set can be useful is evaluating an immune response, such as for identifying responders to checkpoint inhibitor therapies. In some embodiments, the bait set further includes probes for enriching housekeeping genes, such as any subset of gene reported at https://www.tau.ac.il/˜elieis/HKG/can be used a positive controls (having large PFE levels due to continuous expression). In some embodiments, the bait set can further include probes that are designed to enrich for regions of the genome that are not expressed under typical conditions or are not adjacent to transcription start sites as negative controls.

### Discussion

In this study, we introduce EPIC-Seq, a novel approach that leverages cell-free DNA fragmentation patterns to allow non-invasive inference of gene expression and which can be used for a wide variety of clinically relevant applications including tumor detection, subtype classification, response assessment, and analysis of genes with prognostic implications. Compared to EPIC-Seq, the sensitivity of previously described cfDNA fragmentomic techniques and features has been insufficient to resolve expression of individual genes with high fidelity (Jiang, 2018; Sun, 2019; Ramachandran, 2018; Ivanov, 2015; Royer-Pakora, 1995). The approach described here achieves substantially improved performance by leveraging the use of a new entropy-based fragmentomic metric (PFE), as well as higher sequencing depth achieved through targeted capture of promoter regions of genes of interest.

To allow inference of RNA expression levels from cfDNA fragmentomic features by EPIC-Seq, we focused our efforts on capturing features in cfDNA at transcription sites that reflect epigenetically encoded signals from nucleosomal accessibility and positioning since these can be key factors for determining transcriptional output (Smale, 2003; Bernstein, 2005). These fragmentomic signals appeared strongest at promoters of actively expressed genes when profiling cfDNA by whole genome sequencing motivating our TSS capture approach. However, we also observed significant signal at exonic regions of actively expressed genes in whole exome sequencing, suggesting opportunities to extend EPIC-Seq more broadly to study expression of genes of interest. In addition, tissue- and lineage-specificity are also encoded by several other epigenetic signals that can be measured noninvasively including 5mCpG and 5hmCpG modifications and specific histone posttranslational modifications (Wong, 1999; Chim, 2005; Fernandez, 2012; Houseman, 2012; Chan, 2013; Lun, 2013; Ou, 2014; Jensen, 2015; Roadmap Epigenomics, 2015). Several studies have also suggested potential utility in analyzing cell-free RNA, although robust methods to measure this analyte in cancer patients remain to be established, and there is concern that pre-analytical factors may make this challenging. (Koh, 2014; Srinivasan, 2019; Ibarra, 2020; Zhou, 2019; Verwilt, 2020).

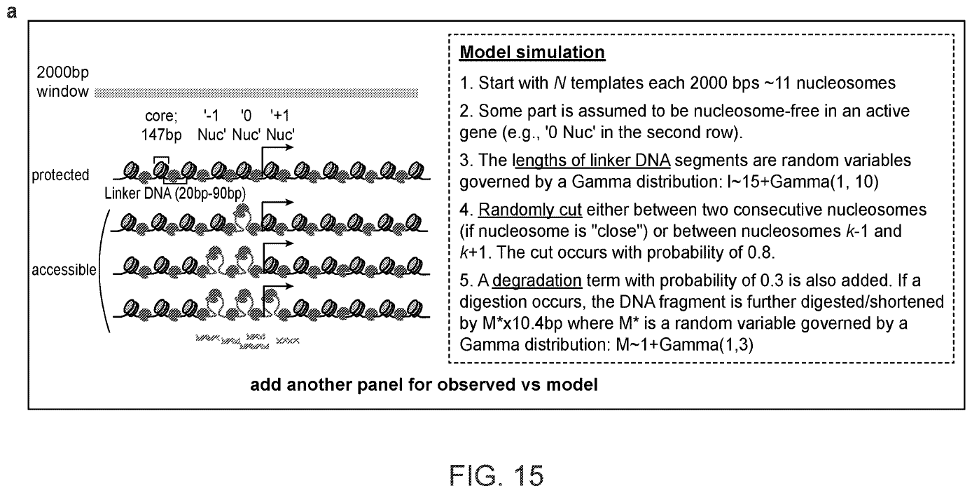

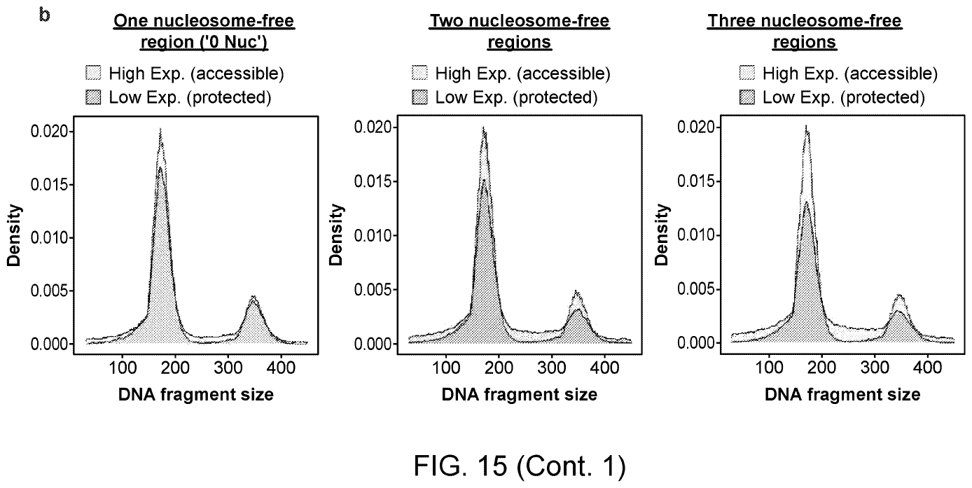

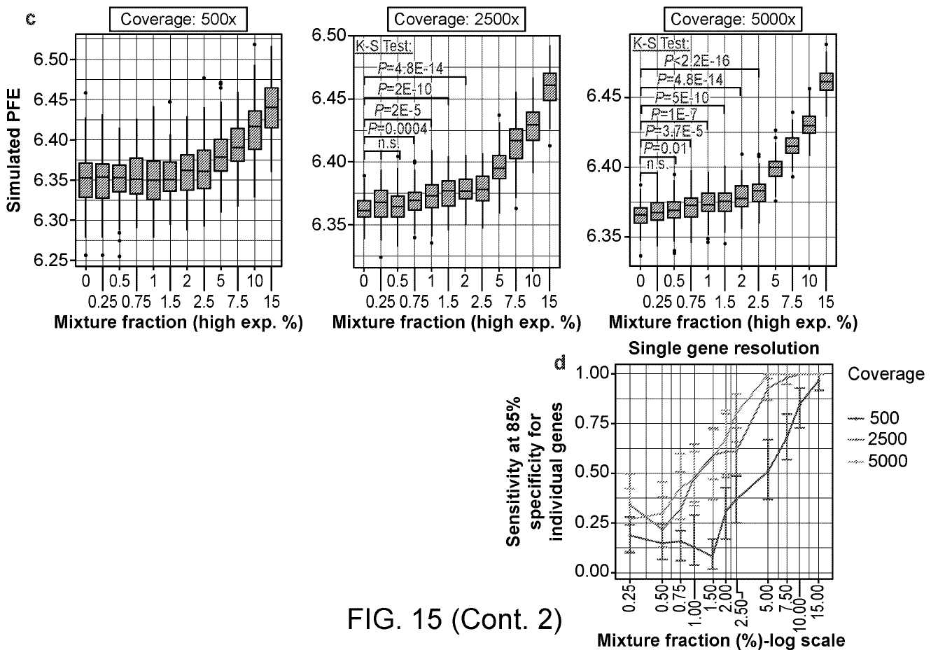

Importantly, we did not observe a significant impact of several pre-analytical factors on cfDNA fragmentation entropy measurements, including blood collection tube types, the time between phlebotomy and plasma isolation, and the number of PCR cycles (FIG. 14). Separately, we observed relatively modest impact of several factors that might confound accuracy of expression estimates derived from cfDNA entropy measurements, including corrections for GC fraction and presence of somatic copy number aberrations (FIG. 14). Finally, we developed a mechanistic framework for how cfDNA fragmentation mirrors activity level of expressed genes in human tissues (FIG. 15a-c). Using this model framework, we used simulations to explore the parameters influencing the likelihood of detection of expression of a given gene of interest within cfDNA as a function of tumor burden (FIG. 15d).

As demonstrated above. EPIC-Seq has potential utility for a wide variety of clinically relevant cancer classification problems. While our study focused on tumor histological classification as a proof-of-concept, the approach we describe here will be likely be broadly generalizable to other tumor types. Importantly, we demonstrate the biological plausibility of the inferred gene expression levels from EPIC-Seq using multiple independent lines of evidence. Specifically, we describe significant correlations of EPIC-Seq signals not only with expectations from tissue transcriptomic profiling, but also with disease burden as measured by total metabolic tumor volume and mutation-based ctDNA analysis. Furthermore, we observed significant correlation of EPIC-Seq signals with therapeutic responses to immunotherapy and chemotherapy, as well as its ability to assess expression of prognostically informative genes.

In our initial application of EPIC-Seq, we focused on the noninvasive histological classification of lung cancers and the molecular classification of aggressive B-cell lymphomas, two common and representative cancer types where such classification is clinically routine but at times fraught by diagnostic challenges. The robust performance that we observed for the accurate classification of each of these tumor subtypes is promising and suggests opportunities for extending this approach more broadly to other cancer types and other pathologies. EPIC-Seq provides a promising avenue for the potential reclassification of carcinomas using non-invasive methods. Separately, the methods we describe could have applications beyond cancer for the noninvasive detection of signals from cell types, tissues, and pathways and pathologies of interest. These include noninvasive strategies to detect tissue injury and ischemia, as well as pharmacodynamic effects on specific therapeutically targeted pathways and toxicity profiles for diverse human tissues that are otherwise difficult to monitor noninvasively (e.g., the brain and gastrointestinal tract), before symptomatic tissue damage occurs.

The method and applications that we describe hold imminent promise in personalized profiling efforts, enabling noninvasive, high-throughput tissue-of-origin characterization with diagnostic, prognostic, and therapeutic potential.

Data and Code availability. The custom EPIC-Seq software code for fragmentomic featurization and gene expression inference from cell-free DNA BAM files can be accessed at Stanford. For each sample profiled in this study by WGS (n=119; including plasma cfDNA n=118, and shorn leukocyte n=1), WES (n=39), and/or EPIC-Seq (n=329), we also provide anonymized fragmentomic data for fragments meeting minimal mapping quality and read FLAGs. These data are summarized across TSS regions by fragment size distributions (as in FIG. 1b).

### Methods

### Human Subjects & Cohorts

Study overview. All samples analyzed in this study were collected with informed consent from subjects enrolled on Institutional Review Board-approved protocols complying with ethical regulations at their respective centers, as detailed below. Fragmentomic features ultimately used for EPIC-Seq were established and initially tested by profiling cfDNA through whole genome sequencing (WGS) and whole exome sequencing (WES), as tabulated in Table 3. These WGS and WES cfDNA profiling data derived from 150 subjects that were either generated for this study (n=64), or from publicly available datasets (n=86).

For initial model development and cfDNA fragmentomic feature selection, we profiled cfDNA from a patient with carcinoma of unknown primary (CUP) by deep WGS to learn the relationships between cfDNA fragmentomic features and expression levels at whole genome scale. After our initial cfDNA profiling of this patient by deep WGS with CUP to build expression predictors from cfDNA, we also profiled two healthy adult subjects by WGS profiling of cfDNA (˜200×) and assessed robustness (Table 3). For initial validation analyses using WGS cfDNA fragmentomics, we also reanalyzed samples from 40 healthy controls and 46 lung cancer patients previously described. (Zviran, 2020).

We then extended our observations from WGS to whole exome sequencing of cfDNA, by deeply profiling 28 plasma specimens from healthy control subjects, and 11 plasma specimens from six patients with extensive stage small-cell lung cancer (SCLC; deep WES). After identification and initial validation by WGS/WES of key cfDNA fragmentomic signals informative for predicting gene expression in the subjects described above. EPIC-seq was then applied to 329 blood samples from 201 cancer patients and 87 healthy adults, as detailed below, and as depicted in FIG. 9. To select genes for the EPIC-Seq capture panel focused on subclassification of lung cancers and lymphomas, we analyzed publicly available gene expression datasets for 1156 lung cancers from The Cancer Genome Atlas and for 381 lymphomas from Schmitz et al., as described below (Scherer, 2016).

Healthy subjects & Non-Cancer controls: To identify and validate cfDNA fragmentomic features informing gene expression prediction, WGS was performed in 30 healthy subjects. These subjects were profiled at varying pre-specified coverage depths (˜300×, n=3; ˜1-5×, n=24; ˜18-25×, n=3), thereby allowing construction of meta-profiles for expression inferences, as described below (see ‘Gene expression inference model’). We separately profiled 94 peripheral blood samples from 87 subjects without cancer using EPIC-Seq. Among these subjects, 35 (40%) qualified for lung cancer screening using low-dose CT (LDCT) due to a history of heavy smoking (≥30 pack years) and age (55-80 years).

### EPIC-Seq Cancer Cohorts

Lung Cancer Cohort: EPIC-Seq was applied to 109 blood samples from 87 patients diagnosed with NSCLC (some with serial samples). Among these patients, 37 (43%) had a histological diagnosis of LUSC, while 50 (57%) patients had LUAD histology. Samples were collected at Stanford University. The University of Texas MD Anderson Cancer Center, or Memorial Sloan Kettering Cancer Centers, with patient characteristics outlined in FIG. 9b. A subset of patients with advanced NSCLC (n=22) was treated with PD-(L)1 blockade-based immune checkpoint inhibition and had serial pre- and on-treatment samples available. These patients had stage IV disease and were treated with PD-(L)1 blockade-based ICI.

DLBCL Cohort: EPIC-Seq was also applied to 126 samples from 114 patients diagnosed with large B-cell lymphoma. Samples were collected at Stanford Cancer Center, CA, USA; MD Anderson Cancer Center, TX. USA; Dijon. France; Novara, Italy; and within the Phase III multicenter PETAL trial (Kurtz, 2018), with baseline characteristics tabulated in FIG. 9b.

Patient with carcinoma of unknown primary (CUP): To assess with high resolution the relationship between fragmentomic features and gene expression we compared deep whole genome sequencing data and RNA-sequencing data of a patient with extremely low tumor burden. Tumor fraction was estimated using a tumor-informed plasma variant detection strategy. First, the patient's tumor germline DNA were prepared for exome capture using the Illumina Nextera Rapid Capture Exome Kit and sequenced on an Illumina NextSeq 500 machine using paired-end sequencing and 75-bp read lengths. Single nucleotide variant (SNV) calling was performed using Mutect and annotated by Annovar. A personalized targeted sequencing panel was generated using 120-bp IDT oligos overlapping SNVs detected in the tumor and applied to the tumor and germline sample. The variant set selected for monitoring consisted of 36 SNVs that both passed tumor/germline quality control filters and were present in at least 10% allele frequency in the tumor. The patient's plasma sample was sequenced on an Illumina NovaSeq machine, achieving a de-duplicated depth of 4000×. The time point used in this study had a monitoring mean allele frequency of 0.056% which is significantly lower than the lower limit of detection of disease at 250× coverage. Results from deep WGS cfDNA profiling of this patient with CUP were then reproduced by the independent WGS profiling of cfDNA (˜200×), and RNA-Seq profiling of matched PBMCs from two healthy adult subjects.

### Clinical Variables

Histopathology. Histological subtypes of each tumor type (SCLC, NSCLC. DLBCL) profiled in this study were established according to clinical guidelines using microscopy and immunohistochemistry and served as ground truths for assessing classification performance by trained pathologists. COO subtypes of DLBCL were assessed based on the Hans classifier per WHO guidelines. (Menon, 2012). For NSCLC and DLBCL subtypes profiled in prior studies by RNA-Seq, we relied on subtype labels from the TCGA (for LUAD vs LUSC subtypes of NSCLC) or from Schmitz el al. (for GCB vs ABC subtypes of DLBCL).

Metabolic tumor volume (MTV) measurement. Pre-treatment tumor MTV was measured from 18FDG PET/CT scans, using semiautomated software tools: For NSCLC, it was done as previously described (Binkley, 2020) via MIM by using PETedge. For DLBCL, three different software tools were used (Beth Israel Fiji, PETRA ACCURATE tool and Metavol) as previously described (Alig, 2021). Regional volumes were automatically identified by the software and confirmed by visual assessment of the expert to confirm inclusion of only pathological lesions.

Clinical Outcomes. Event-free survival (EFS) and overall survival (OS) were calculated from time of treatment initiation. OS events were death from any cause; EFS events were progression or relapse, unplanned retreatment of lymphoma and death resulting from any cause. Patients with NSCLC receiving PD(L)1 directed therapy were labeled as NDB or DCB for ‘experiencing progression or death’ and ‘durable clinical benefit’ within six months, respectively.

### Specimen Collection & Molecular Profiling

Plasma collection & processing. Peripheral blood samples were collected in K2EDTA or Streck Cell-Free DNA BCT tubes and processed according to local standards to isolate plasma before freezing. Following centrifugation, plasma was stored at −80° C. until cfDNA isolation. Cell-free DNA was extracted from 2 to 16 mL of plasma using the QIAamp Circulating Nucleic Acid Kit (Qiagen) according to the manufacturer's instructions. After isolation, cfDNA was quantified using the Qubit dsDNA High Sensitivity Kit (Thermo Fisher Scientific) and High Sensitivity NGS Fragment Analyzer (Agilent).

cfDNA sequencing library preparation. A median of 32 ng was input into library preparation. DNA input was scaled to control for high molecular weight DNA contamination. End repair, A-tailing, and custom adapter ligation containing molecular barcodes were performed following the KAPA Hyper Prep Kit manufacturer's instructions with ligation performed overnight at 4° C. as previously described. (Chabon, 2020; Kurtz, 2018). Shotgun cfDNA libraries were either subjected to whole genome sequencing (WGS) and/or subjected to hybrid capture of regions of interest as described below.

Hybrid capture & Sequencing. Exome capture: For Whole Exome Sequencing (WES), shotgun genomic DNA libraries were captured with the xGen Exome Research Panel v2 (IDT) per manufacturer's instructions with minor modifications. Hybridization was performed with 500 ng of each library in a single-plex capture for 16 hours at 65° C. After streptavidin bead washes and PCR amplification, post-capture PCR fragments were purified using the QIAquick PCR Purification Kit per manufacturer's instructions. Eluates were then further purified using a 1.5×AMPure XP bead cleanup.

Custom capture panels: We used CAPP-Seq to establish ctDNA levels, by genotyping of somatic variants including single nucleotide mutations (Newman, 2016). We used entity-specific CAPP-Seq capture panels for DLBCL or NSCLC (SeqCap EZ Choice, Roche NimbleGen) (Chabon, 2016; Kurtz, 2018), or personalized CAPP-Seq selectors for CUP (IDT), as previously described (Chabon, 2016). Similarly, for EPIC-Seq, we used the SeqCap EZ Choice platform (Roche NimbleGen) to target TSS regions of genes of interest, as described below. Enrichment for WES, CAPP-Seq, and EPIC-Seq was done according to the manufacturers' protocols. Hybridization captures were then pooled, and multiplexed samples were sequenced on Illumina HiSeq4000 instruments as 2×150 bp reads.

RNA-Sequencing. RNA-Seq of PBMCs: The Illumina TruSeq RNA Exome kit was used for RNA-seq library preparation starting from 20 ng of input RNA, per manufacturer's instructions. When using peripheral blood as a source of leukocyte RNA, we used either plasma-depleted whole blood (PDWB) with globin depletion, or enriched PBMCs without globin depletion. In brief, total RNA was fragmented, and stranded cDNA libraries were created per the manufacturer's protocol. The RNA libraries were then enriched for the coding transcriptome by exon capture using biotinylated oligonucleotide baits. Hybridization captures were then pooled, and samples were sequenced on an Illumina HiSeq4000 as 2×150 bp lanes of 16-20 multiplexed samples per lane, yielding ˜20 million paired end reads per case. After demultiplexing, the data were aligned and expression levels summarized using Salmon to GENCODE version 27 transcript models (Patro, 2017). We separately studied tumor RNA-Seq data to identify differentially expressed genes of interest for EPIC-Seq panel design, as described in detail below.

RNA-Seq of lymphoma specimens: Tumor derived RNA was isolated from 2-4, 10 micron thick, formalin-fixed, paraffin embedded (FFPE) scrolls of tumor tissue using the RNA Storm/DNA Storm Combination Kit (Cell Data Sciences, Fremont, CA), according to the manufacturer's protocol. An off-column DNA digestion step was performed using Qiagen's RNase-Free DNase Set followed by column purification using Zymo's RNA Clean & Concentrator kit. RNA concentration was quantified using NanoDrop. The SMARTer Stranded Total RNA-Seq Kit v2 (TaKaRa) was used for RNA-seq library preparation using 50 ng input RNA, according to the manufacturer's protocol. Fragmentation steps were omitted as recommended for RNA isolated from FFPE specimens. Yield and fragment size of libraries were assessed using Qubit (dsDNA HS assay kit) and TapeStation. Libraries were sequenced on an Illumina HiSeq4000 or NovaSeq6000, respectively, with 2×150 bp paired-end reads.

### Data Analysis Methods

Mapping, deduplication, and quality control of TSS sites and samples. FASTQ files were demultiplexed using a custom pipeline wherein read pairs were considered only if both 8-bp sample barcodes and 6-bp UIDs matched expected sequences after error-correction (Chabon, 2020). After demultiplexing, barcodes were removed, and adaptor read-through was trimmed from the 3′ end of the reads using fastp to preserve short fragments (Chen, 2018). Fragments were aligned to human genome (hg 19) using BWA; importantly, we disabled the automated distribution inference in BWA ALN to allow inclusion of shorter and longer cfDNA fragments that would otherwise be anomalously flagged as improperly paired. We removed PCR duplicates using a customized barcoding approach, which combines endogenous and exogenous unique molecular identifiers (UMIDs), including cfDNA fragment start and end positions, as well as pre-specified UMIDs within ligated adapters into account. To allow coverage uniformity for comparisons, we down-sampled data to 2000× depth using ‘samtools view -s’. Since in-silico simulations showed >500× sequencing depth may be required for achieving reasonable correlations between entropy and expression, we considered any samples not meeting this depth threshold (median depth) as failing quality control (QC). Any samples whose cfDNA fragment length density mode was below 140 or above 185 were also removed, since the expected fragment length density mode is 167 (corresponding to the chromatosomal DNA length). Together, these two criteria removed 21 samples as not meeting QC. To identify and censor noisy sites among the 236 TSS regions profiled by our EPIC-Seq panel, we profiled 23 controls (Table 5), allowing us to identify and remove stereotyped regions with reproducibly low TSS coverage (i.e., any site with CPM less than one third of uniformly distributed coverage across the TSSs in the selector. i.e.,

$$\frac{{10}^{6}}{236} \times \frac{1}{3} ,$$

in more than 75% of controls). This removed two TSS sites in FOXO1 and SFTA2 as not meeting QC.

To guarantee adequate quality of fragments entering analysis, we required mapping quality (MAPQ, k) of >30 or >10 in the WGS and EPIC-Seq data, respectively (using ‘samtools view -q k -F3084’). The more lenient EPIC-seq MAPQ threshold was qualified by more stringent mappability and uniqueness requirements already imposed on the TSS regions selected during EPIC-seq selector design. We also limited the analysis to reads with the following BAM FLAG set: 81, 93, 97, 99, 145, 147, 161, and 163. To ensure removal of non-unique fragments, reads with duplicate names were censored.

Fragmentomic feature extraction & summarization. We considered 5 cfDNA fragmentomic features at TSS regions and then compared each of these features to gene expression, including Window Protection Score (WPS; Snyder, 2016). Orientation-aware CfDNA Fragmentation (OCF; Sun, 2019), Motif Diversity Score (MDS; Jiang, 2020). Nucleosome depleted region score (NDR; Ulz, 2016), and Promoter Fragmentation Entropy (PFE, introduced here). MDS, NDR. OCF, and WPS were each computed as per the conventions of the originally describing studies with minor modifications, as detailed below.

Motif diversity score (MDS). We performed end-motif sequence analysis of individual cfDNA fragments to assess the distribution of nucleotides among the first few positions for the reads of each read pair, as previously described (Jiang, 2020). This was performed by computationally extracting the first four 5′ nucleotides of the genomic reference sequence for each sequence read, resulting in a 4-mer sequence motif. MDS was then computed as the Shannon index of the distribution across 256 motifs (4-mers) at each TSS site, when considering fragments overlapping the 2 kb window flanking each TSS. Of note, the first four 3′ nucleotides were not used as these may be altered by end-repair during library preparation and may not reflect the native genomic sequence.

Nucleosome depleted region score (NDR). To guard against variations in depth across the genome, including from GC-content variation or somatic copy number changes, depth was normalized within each 2-kilobase window flanking each TSS (−1000 to +1000 bp) in counts per million (CPM) space. We denote this normalized measure as nucleosome depleted region score. NDR, for each TSS.

Promoter fragmentation entropy (PFE). Shannon entropy was used to summarize the diversity in cfDNA fragment size values in the vicinity of each TSS site (−1 Kbps (5′; upstream) to +1 Kbps (3′; downstream)). We defined 201 size-bins (from b₁=100 bps to b₂₀₁=300 bps) and estimated the density by the maximum-likelihood, i.e., \hat{p}=[p₁, . . . , p₂₀₁] with

$$\hat{{p}_{ι}} = \frac{{n}_{i}}{n}$$

where nᵢ and n denote the number of fragments with length bᵢ and total number of fragments at the TSS, respectively. Shannon's entropy was calculated as −Σ\hat{p}_{ι} log₂ \hat{p}_{ι}.

To account for variations in sequencing depth between samples as well as other hidden factors impacting overall cfDNA fragment length distributions as potential confounders, we performed normalization steps using a Bayesian approach through a Dirichlet-multinomial mixture (DMM) model:

For a given sample, we first built a sample-wide fragment length distribution using the multinomial maximum likelihood estimation. Importantly, to minimize the impact of gene expression on this background fragment length distribution, we focused on the two 250 bp regions within the 2 kb window with the longest distance from the center of the TSS: (a) −1 Kbps to −750 bps (upstream) and (b) from +750 bps to +1 Kbps (downstream). Fragment length densities across the 201 size-bins were then used as parameter vector a of a Dirichlet distribution with α0=20. For each TSS, we then updated the sample-wide background distribution to calculate the sample adjusted and gene-specific posterior of the Dirichlet distribution based on fragment counts in the 201 size bins within the 2 kb region around the TSS:

$$Dir ( {α}^{*} = α + [ {\hat{n}}_{1} , … , {\hat{n}}_{201} ] )$$

From Dir(α*), we sampled 2000 fragment length distributions, and calculated the corresponding Shannon's entropies. Each value was then compared to the Shannon entropies of five randomly selected background gene sets, denoted as e₁, e₂, e₃, e₄, and e₅. PFE was defined as the likelihood of the gene-specific entropy (uncertainty class) exceeding the control gene entropies by (1+k) fold for all n=5 groups with a random variable k. Here, we used a Gamma distribution for k˜Γ(s=0.5, r=1), where Γ is the Gamma distribution with shape s and rate r. PFE therefore is a measure for the excess diversity in the fragment length distribution at a given TSS of interest compared to control genes, and is formally defined as

$$PFE ( TSS ) := {E}_{k} [ {\sum }_{i : 1 - 5} {P}^{*} ( {e}_{TSS} > ( 1 + k ) \times {e}_{i} ) ]$$

where Eₖ[.] denotes the expected value with respect to the excess parameter k, and P* is the probability with respect to the Dirichlet distribution Dir(α*) approximated by the 2,000 draws.

Pre-Analytical Factors. We examined robustness of PFE against pre-analytical biases such as blood collection tubes, processing time and number of PCR cycles. To confirm that the type of collection tube does not confound the PFE, we collected blood from three healthy donors in K2EDTA and Streck BCT tubes and compared PFE in the TSSs in the EPIC-Seq selector, and measured concordance between the two using Pearson correlation. To evaluate the robustness against processing time, we used a cohort of DLBCL patients captured by a CAPP-Seq lymphoma panel and calculated PFE for the regions in the panel and summarized each patient by the median PFE across these regions (Alig, 2021). We compared samples grouped by the number of interval days before processing, and measured correlation between median PFE and time at room temperature. We also tested effect of number of PCR cycles on PFE, by performing additional PCR cycles on cfDNA libraries from four healthy donors. Here, we compared the PFEs of regions in our NSCLC panel and measured Pearson correlation in PFEs with or without the additional PCR cycles.

cfDNA Fragmentomic Analysis by WES Profiling

Whole exome PFE analysis. For whole exome analyses (in FIG. 1g-h, FIG. 2d-h, and FIG. 8g-i), we used the raw Shannon entropy (as described in ‘Fragment length diversity calculation using Shannon entropy’) at any given gene, after transforming it into a z-score, using a cohort of 39 cfDNA WES profiles (each with 200-400× depth), including 28 plasma samples from healthy adult controls, and 11 plasma samples from patients with SCLC. To account for differences in depth in the cohort for normalization, we considered meta-profiles of 5 samples to achieve comparable depths as those initially used to relate PFE and gene expression levels when relying on WGS (˜2000×).

Small cell lung cancer (SCLC) gene signatures. A SCLC-specific gene signature was generated using a previously described RNA-Seq dataset of 81 surgically resected primary tumors (George, 2015). To identify genes highly expressed in SCLC tumors but not circulating leukocytes (i.e., ‘SCLC High Genes’, n=118), we selected genes with mean TPM>50 in these SCLC tumors and mean TPM<0.5 in PBMCs from GSE107011 (n=13). We limited our analyses to protein coding genes, and renormalized expression levels to 1E6 after removal of individual genes with TPM>100,000 (n=16,865 genes). Conversely, we selected ‘SCLC Low Genes’ (n=20) with TPM<0.5 in SCLC tumors and >50 in PBMC. Using the deep whole-exome cfDNA profiles described above, we then calculated the mean Shannon entropy of first exons (i.e., as an estimate for the residual PFE captured by exon 1 fragments) for each of the two SCLC signature sets, after subtracting the mean PFE of a set of control genes used throughout the whole-genome analyses. These two gene sets, which were originally defined in tumors and PBMCs by RNA-Seq were then compared for their mean PFE in cfDNA of a set of SCLC patients and control subjects that we profiled by deep WES. Next, we defined a ‘SCLC Signature Score’ as the difference between the ‘High’ and ‘Low’ sets. This allowed us to compare cfDNA profiles of SCLC cases versus healthy controls for the discriminating power of the ‘SCLC Score’ through calculation of the area under curve (AUC) of a receiver-operator curve (ROC). We separately identified differentially expressed genes directly from cfDNA, by comparing PFEs of SCLC cases versus healthy adult controls in a volcano plot analysis. Specifically, we used t-tests with FDR threshold of 0.05 and mean PFE difference of at least 0.1. Here again, we compared the mean expression level in TPM for these differentially expressed genes in RNA-Seq data from SCLC tumors and PBMC samples described above. Overlap between two ‘SCLC High’ gene sets identified by either tumor RNA-Seq or by cfDNA WES profiling was performed using the hypergeometric test.

Genotyping of somatic copy number variants (CNVs). Genomic copy number alterations in healthy and SCLC cfDNA samples profiled by deep WES were identified using CNVKit version 0.9.8. (U, 2014). Raw genomic coverage was calculated from deduplicated ‘bam’ files for each sample considering on-target (IDT xGen Exome Research Panel v2) as well as off-target regions. To correct for potential biases in capture efficiency and GC content, a pooled per-region reference was generated from 5 healthy cfDNA samples that were held-out. The remaining healthy and SCLC samples were then normalized utilizing this pooled reference, with discrete copy number segments inferred utilizing the default circular binary segmentation algorithm (Venkatraman. 2007). Corresponding Log 2 copy number values for each segment were then utilized in further analyses. We considered whether CNVs might disproportionately impact PFE estimates in two ways. First, we considered whether the PFE difference in genes falling within amplifications versus deletions is significantly different in SCLC than in healthy controls subjects profiled by deep cfDNA WES. Second, using Fisher's exact test, we tested whether genes inferred to be highly expressed in SCLC cfDNA were significantly more likely to fall in amplifications, and conversely, whether those inferred as lowly expressed were more likely to be deleted.

Consideration of GC Correction. Two healthy control cfDNA samples were profiled by deep whole genome sequencing. For these two subjects, we also profiled the matched PBMC by RNA-Sequencing. We then compared the predicted values from cfDNA against observed values from RNA-Seq for each of the different GC-correction scenarios and tested concordance. We tested the impact of correcting for GC-content of TSS regions on gene expression model accuracy in several ways. We considered four scenarios were studied when correcting features using the GC values for NDR and PFE: PFE alone corrected, NDR alone corrected, both corrected, and neither corrected. The correction was performed using a LOESS function with a span of 0.5. The concordance was evaluated using three metrics: Pearson correlation, Spearman correlation, and root-mean-square error (RMSE). Since none of these GC-correction approaches significantly improved correlations or reduced associated error profiles when considering reproducibility across cfDNA samples, we opted not to correct for variability in GC content across the TSS regions of different genes.

A gene expression model for predicting RNA output from TSS cfDNA fragmentomic features. To infer RNA expression levels from cfDNA fragmentation profiles at TSS regions of genes across the transcriptome, we built a prediction model using two features. PFE and NDR. Of note, among the 5 fragmentomic features considered, these indices demonstrate highest individual correlations as well as complementarity. For training, we employed one cfDNA sample sequenced to high coverage depth by WGS. We performed RNA-Seq on the PBMC of five healthy subjects and used the average across three of these individuals as the ‘reference expression vector’. Next, to achieve a higher resolution at the core promoters, we grouped every 10 genes, based on their expression in our reference RNA-seq vector. After removing genes used as background for calculating PFE, a total of 1.748 groups (of 10 genes each) remained. We then pooled all the fragments at the extended core promoters (−1 Kb/+1 Kb around the transcription start sites) of the genes within each group and extracted the two features: NDR and PFE. We then normalized the two features by 95% quantile over the background genes, where for PFE the normalization factor is

$$= min ( 1 , \frac{PFE}{Γ ( \frac{Q ( \{ PFE \} , 95 )}{{PFE}_{{B}_{g}}} ; 0.5 , 1 )} ) and = \frac{NDR}{{NDR}_{{B}_{g}}} ,$$

where Q(.,k) denotes the k^{th} quantile. By bootstrap resampling, we then built 600 ensemble models: 200 univariable PFE-alone-models m_{PFE,1}, m_{PFE,2}, . . . , m_{PFE,200}, 200 univariable NDR-alone-models m_{NDR,1}, m_{NDR,2}, . . . , m_{NDR,200} and 200 NDR-PFE integrated models m_{int,1}, m_{int,2}, . . . , m_{int,200}.

To transfer this expression prediction model—which was originally derived from WGS—to the targeted TSS space (EPIC-seq), we evaluated each of the 600 models above, by measuring its root mean squared error (RMSE) on two held out healthy subjects. For each of these two healthy subjects, we compared the cfDNA profile by EPIC-seq to the corresponding PBMC transcriptome profile by RNA-Seq from the same blood specimen and computed the RMSE for each of the 600 ensemble models. The weight of each model was then proportionally scaled by the inverse RMSE of that model, with the final score then calculated as the linear sum of 600 models, weighted as described above.

### EPIC-Seq Panel Design

Identification of cancer type-specific genes. We downloaded TCGA and DLBCL gene expression data in the form of RNA-Seq FPKM-UQ for all individuals using the GDC API. After removing samples from individuals with a history of more than one type of malignancy, we divided the remaining samples into two separate cohorts for training and validation (70% and 30% of each cancer type respectively). In the training set for each cancer type, median gene expression (FPKM-UQ) was calculated and protein coding genes in the upper 15th quantile were considered as highly expressed genes. To remove potentially confounding effects in cfDNA from variation in blood cells, we excluded genes within the upper 5^{th} quantile of expression in peripheral blood, when considering whole-blood transcriptome profiles from GTEx.

Gene selection for EPIC-Seq targeted sequencing panel design. We considered NSCLC and DLBCL, with known molecular subtypes exhibiting distinct gene expression profiles. Cancer-specific genes for LUAD, LUSC, and DLBCL were included. To find subtype-specific genes in NSCLC, we performed differential expression analysis using the DESeq2 package in R Bioconductor to distinguish LUAD and LUSC tumor transcriptomes from the TCGA. For the lymphoma analysis, a list of genes previously shown as differentially expressed between ABC and GCB subtypes according to RNA-Seq gene expression data was used³⁹. In addition to these DLBCL and NSCLC specific genes, we included 50 genes from the LM22 gene set capturing variation in peripheral blood leukocyte counts (Newman, 2019). Together these and other control genes contributed to a total of 179 unique genes, with each gene contributing one or more TSS regions to EPIC-Seq totaling 236 targeted TSS regions.

### EPIC-Seq Classification Analyses and Machine Learning

Distinguishing lung cancer (EPIC-Lung classifier). The EPIC-Lung classifier was trained to distinguish lung cancer from non-cancer subjects. All the TSSs for immune cell type and NSCLC histology classification were used in this classifier. For genes with multiple TSS regions, in each iteration of cross-validation, we first combined TSS regions with intra-gene correlation exceeding 0.95 and capturing the mean. For those with correlation less than 0.95, we preserved individual TSS regions as independent reporters. This resulted in 139 features in the model and 143 samples (67 lung cancer cases and 71 controls). We then trained an [symbol] —regularized logistic regression model (‘elastic net’ with α=0.9) and an optimal A obtained by cross-validation. The full model was evaluated through a leave-one-batch out (LOBO) model. Here, every batch contained at least one sample, and representing a set of samples that were either captured and/or sequenced together in one NGS sequencing lane.

Subclassification of NSCLC (EPIC-NSCLC-Subtype). A NSCLC histology subtype classifier was designed to distinguish the two major subtypes of non-small cell lung cancer, i.e., lung adenocarcinoma (LUAD) and lung squamous cell carcinoma (LUSC). As in the model in ‘EPIC-Lung classifier’, the classification model employs elastic net with α=0.9, with multiple TSS sites corresponding to one gene being merged. The performance of this classifier was evaluated via leave-one-out (LOO) analysis. The classifier was trained using 80 features with 67 samples (36 LUADs and 31 LUSCs). To evaluate performance, classification accuracy with equal weights was calculated.

Biological plausibility of classifier coefficients. We assessed the significance of the model coefficients in the NSCLC histology classifier from plasma cfDNA using EPIC-Seq and their concordance with prior design from tumor transcriptomes using RNA-Seq. Specifically, we compared nonzero coefficients from the elastic net model from cfDNA profiling, and then performed a t-test for the LUAD genes coefficients vs LUSC genes coefficients.

EPIC-seq lung dynamics score for the ICI treated patients. To predict benefit from immune checkpoint inhibitors, we first identified the differentially expressed TSSs in a discovery pre-treatment cohort (non-ICI; lung cancer vs normal). We then nominated the following TSS regions from genes with Bonferroni-corrected P<0.25 with a 1-sided t-test: (FOLR1 TSS #3, ITGA3 TSS #1, LRRC31 TSS #1, MACC1 TSS #1, NKX2-1 TSS #2, SCNN1A TWSS #2 SFTPB TSS #1, WFDC2 TSS #1, CLDN1 TSS #1, FSCN1 TSS #1, GPC1 TSS #1, KRT17 TSS #1, PFN2 TSS #1, PKP1 TSS #1, S100A2 TSS #1, SFN TSS #1, SOX2 TSS #2. TP63 TSS #2). Denoting the expression levels of these genes by

$${ξ}_{{t}_{0}} = ( {x}_{1}^{{t}_{0}} , … , {x}_{k}^{{t}_{0}} ) and {ξ}_{{t}_{1}} = ( {x}_{1}^{{t}_{1}} , … , {x}_{k}^{{t}_{1}} )$$

for time point t₀ and t₁, respectively, we defined (fold-change) statistics

$$s ( {ξ}_{{t}_{0}} , {ξ}_{{t}_{1}} ) = log ( \frac{\overline{{ξ}_{{t}_{1}}}}{{ξ}_{{t}_{0}}} )$$

where (̅.̅)̅ is used to denote averaging the vector elements. For each patient, we then empirically derived a null distribution for the s statistics by randomly selecting k sites from the EPIC-Seq selector. An empirical left-sided P-value was then calculated to measure response to therapy. The EPIC-seq dynamics score was then defined as the logarithm (base 10) of these empirical P-values.

Distinguishing lymphoma (EPIC-DLBCL classifier). This classifier was trained to distinguish DLBCL from non-cancer subjects using elastic-net, with regularization parameters being set as in ‘EPIC-Lung classifier’. The dataset used for LOBO cross-validation comprised 129 features and 167 samples (91 DLBCL cases and 71 controls).

Subclassification of DLBCL cell-of-origin (EPIC-DLBCL-COO). For the classification of DLBCL COO, we defined a GCB score as follows: (1) within a leave-one-out cross-validation framework, we first standardized each gene expression (i.e. the Z-score) and converted the Z-scores into probabilities, and then (2) defined a COO score as

$$- log 10 ( \frac{\sqrt[1 / \vert GCB \vert]{{\prod }_{i ∈ GCB} {p}_{i}}}{\sqrt[1 / \vert ABC \vert]{{\prod }_{i ∈ ABC} {p}_{i}}} ) .$$

Gene sets for each subtype were defined as originally selected in the EPIC-Seq selector design for DLBCL classification. To evaluate performance, we measured the concordance between EPIC-Seq scores and (1) genetic COO classification scores obtained from CAPP-Seq (Scherer, 2016), as well as (2) labels from Hans immunohistochemical algorithm. Finally. (3) for a subset of patients with available matched formalin-fixed paraffin embedded (FFPE) tumor specimens, we compared EPIC-Seq COO scores for the GCB-signature from cfDNA against the corresponding GCB-scores from RNA-Seq profiling of tumor biopsies.

Mechanistic modeling of nucleosome accessibility. In relying on several assumptions from the structural studies and the chromatin literature, we developed a mechanistic model linking nucleosome accessibility at TSS regions to corresponding cfDNA fragmentation profiles, and associated expression levels (depicted in FIG. 15). Specifically, we assumed (1) starting with N=5,000 genome templates of size 2 kb centered at the transcription start site (TSS), wherein each has the ‘+1 nucleosome’ is well-positioned at the TSS (i.e., position zero). Then, (2) within this 2000 bp region with, we assumed that a total of 11 nucleosomes can be present and spaced at −180 bp inter-nucleosome distances. When constrained by the well-positioned nucleosome at the TSS. (3) the position of other nucleosomes is determined by 147 (the length of DNA in the core octamer particle) plus a variably sized linker DNA segment. Here, we modeled the linker DNA size flanking each nucleosome as a random variable, defining as Linker_{DNA}_{ᵢ}=20+Gamma(1,10). Within this approach, the position of the 3′ nucleosomes downstream of +1 nucleosome is determined as posᵢ=Σ_{1≤j≤i-1}(147+Linker_{DNA}_{ᵢ}) for i≥2 with pos₁=0. The position of 5′ nucleosomes upstream of +1 nucleosome is determined as posᵢ=Σ_{j≤i≤0}−(147+Linker_{DNA}_{ᵢ}). We assumed that (4) the cut site positions are located either (a) within the linker segments (i.e., within the interval (posᵢ+147, posᵢ₊₁)) with a cutting probability, p_{cut} (in our simulations p_{cut}=0.7), or (b) anywhere within the interval (posᵢ₋₂+147, posᵢ) with p_{cut}=0.7 for accessible sites (e.g., at i=1 for active genes). A cfDNA fragment length was then generated by cutting the initial template at the cut sites. Finally, we assumed (5) a secondary ‘degradation event’ to generate smaller, subnucleosomal fragments as a Bernoulli process, and occurring with a probability, p_{degrade}, (set to 0.2 here). Within this secondary degradation process, in considering the ˜10 bases per helical DNA turn, we thus shortened the cfDNA fragment according to the random variable d=1+10×round(x), where x˜Gamma(1,3), i.e., l_{digested}=l−d. We then plotted the associated size distributions for these pseudo-cfDNA molecules generated in silico, allowing us to compare profiles for genes with high versus low expression, when assuming their promoters to have variably accessible TSS with 1-3 nucleosome-free regions.

Simulating mixtures using the mechanistic model. To simulate the effect of variable quantities of tumor-derived molecules in plasma cfDNA, we created mixtures controlled by a mixing factor r≥0. For each r, we begin with N genomes, equivalent to coverage depth here. We then randomly selected a subset of genomes (Nₜᵤₘₒᵣ), using a Binomial distribution with probability of T, and assigned them to either of 3 scenarios where the TSS is variably accessible, assuming that each of the 3 scenarios are equally probable (as depicted in FIG. 15a). The remaining cfDNA molecules (i.e., N−Nₜᵤₘₒᵣ) are then assumed to be fully nucleosome protected, and thus not accessible (as depicted in FIG. 15a). We then mix the resulting fragments to calculate the associated entropy (PFE) at these modeled TSS. For the simulations, we varied the variable τ from zero to 0.15 and generated 100 simulated mixtures. To summarize the results, for each nonzero τ, we compared the entropy values with the inactive scenario (i.e., τ=0) and via ROC analysis, allowing us to determine the sensitivity corresponding to 85% specificity. To assess the effect of sequencing depth, the entire analysis was re-performed for three different unique cfDNA coverage depths. N∈{500,2500,5000}. Within each of these 3 sequencing depths, we identified ctDNA mixture (%) levels that were significantly discernable by Kolmogorov-Smirnov test, when comparing the simulated PFE of the cfDNA mixture including variable levels of circulating tumor DNA (ctDNA) against a pure mixture devoid of ctDNA.

Statistical and patient survival analysis. Throughout the study, associations between known and predicted variables were measured by Pearson correlation (r) or Spearman correlation (ρ) depending on data type. Whenever data were depicted as box-and-whisker plots, boxes span the inter-quartile range (IQR), while the median is horizontally marked with a line in each box, and whiskers span the 1.5 IQRs. When data were normally distributed, group comparisons were determined using t-test with unequal variance or a paired t-test, as appropriate; otherwise, a two-sided Wilcoxon test was applied. To test for trend in continuous variables across categorical groups, Jonckheere's trend test was used as implemented in the clinfun R package. Correction for multiple hypothesis testing was performed using the Bonferroni method. Results with two-sided P<0.05 were considered significant. Statistical analyses were performed with R 4.0.1. Confidence intervals (CI) were calculated by re-sampling with replacement (i.e., bootstrapping). Receiver operating characteristic (ROC) curve analyses were performed using the R package pROC. Survival analyses were performed using R package survival. When dichotomized, Kaplan-Meier estimates were used to plot stratified survival curves and statistical significance was evaluated by log-rank test. Otherwise, Cox proportional-hazards models were fitted to the data to determine the significance of each co-variate using Wald log-likelihood testing to assess significance. The differential expression (or PFE) analyses are summarized by statistical significance (via FDR adjustment by p.adjust R function) and change magnitude, which are visualized by volcano plots.
Table 1. Exemplary probes used for detection of lymphoid diseases.
Table 2. Exemplary probes used for detection of immune diseases
Table 3. Whole-genome (n=116) and whole-exome (n=39) sequencing of cell-free DNA samples were used for PFE, training the gene expression inference model and its validation. The WGS data were either profiled in this study (n=30) or downloaded from Zviran et al. (EGA accession number EGAS00001004406). Cell-free DNA from 226 subjects were profiled using EPIC-seq.
Table 4: Gene groups—average expression values of genes in each group in PBMC, normalized PFE. OCF, WPS, and MDS in the deep WGS sample.
Table 5. TSSs in the EPIC-seq selector. Each row corresponds to one TSS in the EPIC-seq sequencing panel (‘selector’).
Table 6. EPIC-Seq samples clinical characteristics and scores corresponding to different classifiers. EPIC-Seq was applied to 373 samples, of which 329 passed the QC steps, and were used to show the utility of the inferred gene expression in different applications: cancer detection, tumor subtype classification, and patient response to treatment prediction

### REFERENCES

1. Jahr, S. et al. DNA fragments in the blood plasma of cancer patients: quantitations and evidence for their origin from apoptotic and necrotic cells. Cancer Res 61, 1659-1665 (2001).
2. Lo, Y. M. et al. Maternal plasma DNA sequencing reveals the genome-wide genetic and mutational profile of the fetus. Sci Transl Med 2, 61ra91 (2010).
3. Heitzer, E., Auinger, L. & Speicher, M. R. Cell-Free DNA and Apoptosis: How Dead Cells Inform About the Living. Trends Mol Med 26, 519-528 (2020).
4. Newman, A. M. et al. An ultrasensitive method for quantitating circulating tumor DNA with broad patient coverage. Nat Med 20, 548-554 (2014).
5. Phallen, J. et al. Direct detection of early-stage cancers using circulating tumor DNA. Sci Transl Med 9 (2017).
6. Cohen, J. D. et al. Detection and localization of surgically resectable cancers with a multi-analyte blood test. Science 359, 926-930 (2018).
7. Cristiano, S. et al. Genome-wide cell-free DNA fragmentation in patients with cancer. Nature 570, 385-389 (2019).
8. Heitzer, E., Haque, I. S., Roberts, C. E. S. & Speicher, M. R. Current and future perspectives of liquid biopsies in genomics-driven oncology. Nat Rev Genet 20, 71-88 (2019).
9. Chabon, J. J. et al. Integrating genomic features for non-invasive early lung cancer detection. Nature 580, 245-251 (2020).
10. Van Opstal, D. et al. Origin and clinical relevance of chromosomal aberrations other than the common trisomies detected by genome-wide NIPS: results of the TRIDENT study. Genet Med 20, 480-485 (2018).
11. Fan, H. C. et al. Non-invasive prenatal measurement of the fetal genome. Nature 487, 320-324 (2012).
12. Knight, S. R., Thorne, A. & Lo Faro, M. L. Donor-specific Cell-free DNA as a Biomarker in Solid Organ Transplantation. A Systematic Review. Transplantation 103, 273-283 (2019).
13. Chaudhuri, A. A. et al. Early Detection of Molecular Residual Disease in Localized Lung Cancer by Circulating Tumor DNA Profiling. Cancer Discov 7, 1394-1403 (2017).
14. Lennon, A. M. et al. Feasibility of blood testing combined with PET-CT to screen for cancer and guide intervention. Science 369 (2020).
15. Zviran, A. et al. Genome-wide cell-free DNA mutational integration enables ultra-sensitive cancer monitoring. Nat Med 26, 1114-1124 (2020).
16. Lo, Y. M. et al. Presence of donor-specific DNA in plasma of kidney and liver-transplant recipients. Lancet 351, 1329-1330 (1998).
17. Snyder, T. M., Khush, K. K., Valantine, H. A. & Quake, S. R. Universal noninvasive detection of solid organ transplant rejection. Proc Natl Acad Sci USA 108, 6229-6234 (2011).
18. Lehmann-Werman, R. et al. Identification of tissue-specific cell death using methylation patterns of circulating DNA. Proc Natl Acad Sci USA 113. E1826-1834 (2016).
19. Jiang, P. et al. Preferred end coordinates and somatic variants as signatures of circulating tumor DNA associated with hepatocellular carcinoma. Proc Natl Acad Sci USA 115, E10925-E10933 (2018).
20. Sun, K. et al. Orientation-aware plasma cell-free DNA fragmentation analysis in open chromatin regions informs tissue of origin. Genome Res 29, 418-427 (2019).
21. Sadeh, R. et al. Ch1P-seq of plasma cell-free nucleosomes identifies gene expression programs of the cells of origin. Nat Biotechnol (2021).
22. Lui, Y. Y. et al. Predominant hematopoietic origin of cell-free DNA in plasma and serum after sex-mismatched bone marrow transplantation. Clin Chem 48, 421-427 (2002).
23. Fleischhacker, M. & Schmidt, B. Circulating nucleic acids (CNAs) and cancer—a survey. Biochim Biophys Acta 1775, 181-232 (2007).
24. Ramachandran, S., Ahmad, K. & Henikoff, S. Transcription and Remodeling Produce Asymmetrically Unwrapped Nucleosomal Intermediates. Mol Cell 68, 1038-1053 e1034 (2017).
25. Snyder, M. W., Kircher, M., Hill, A. J., Daza, R. M. & Shendure, J. Cell-free DNA Comprises an In Vivo Nucleosome Footprint that Informs Its Tissues-Of-Origin. Cell 164, 57-68 (2016).
26. Ivanov, M., Baranova, A., Butler, T., Spellman, P. & Mileyko, V. Non-random fragmentation patterns in circulating cell-free DNA reflect epigenetic regulation. BMC Genomics 16 Suppl 13. S1 (2015).
27. Ulz, P. et al. Inferring expressed genes by whole-genome sequencing of plasma DNA. Nat Genet 48, 1273-1278 (2016).
28. Wu, J. et al. Decoding genetic and epigenetic information embedded in cell free DNA with adapted SALP-seq. Int J Cancer 145, 2395-2406 (2019).
29. Jiang, P. et al. Lengthening and shortening of plasma DNA in hepatocellular carcinoma patients. Proc Natl Acad Sci USA 112, E1317-1325 (2015).
30. Underhill, H. R. et al. Fragment Length of Circulating Tumor DNA. PLoS Genet 12, e1006162 (2016).
31. Mouliere, F. et al. Enhanced detection of circulating tumor DNA by fragment size analysis. Sci Transl Med 10 (2018).
32. Ulz, P. et al. Inference of transcription factor binding from cell-free DNA enables tumor subtype prediction and early detection. Nat Commun 10, 4666 (2019).
33. Moss, J. et al. Comprehensive human cell-type methylation atlas reveals origins of circulating cell-free DNA in health and disease. Nat Commun 9, 5068 (2018).
34. Weintraub, H. & Groudine, M. Chromosomal subunits in active genes have an altered conformation. Science 193, 848-856 (1976).
35. Jiang, P. et al. Plasma DNA End-Motif Profiling as a Fragmentomic Marker in Cancer, Pregnancy, and Transplantation. Cancer Discov 10, 664-673 (2020).
36. Adalsteinsson, V. A. et al. Scalable whole-exome sequencing of cell-free DNA reveals high concordance with metastatic tumors. Nat Commun 8, 1324 (2017).
37. Cancer Genome Atlas Research, N. Comprehensive molecular profiling of lung adenocarcinoma. Nature 511, 543-550 (2014).
38. Cancer Genome Atlas Research, N. Comprehensive genomic characterization of squamous cell lung cancers. Nature 489, 519-525 (2012).
39. Schmitz, R. et al. Genetics and Pathogenesis of Diffuse Large B-Cell Lymphoma. N Engl J Med 378, 1396-1407 (2018).
40. Newman, A. M. et al. Robust enumeration of cell subsets from tissue expression profiles. Nat Methods 12, 453-457 (2015).
41. Newman, A. M. et al. Integrated digital error suppression for improved detection of circulating tumor DNA. Nat Biotechnol 34, 547-555 (2016).
42. Maloney, D. G. et al. Phase I clinical trial using escalating single-dose infusion of chimeric anti-CD20 monoclonal antibody (IDEC-C2B8) in patients with recurrent B-cell lymphoma. Blood 84, 2457-2466 (1994).
43. Puglisi, F. et al. Prognostic value of thyroid transcription factor-1 in primary, resected, non-small cell lung carcinoma. Mod Pathol 12, 318-324 (1999).
44. Ferlay, J. et al. Cancer incidence and mortality worldwide: sources, methods and major patterns in GLOBOCAN 2012. Int J Cancer 136, E359-386 (2015).
45. Torre, L. A., Siegel, R. L. & Jemal, A. Lung Cancer Statistics. Adv Exp Med Biol 893, 1-19 (2016).
46. Travis, W. D. et al. The 2015 World Health Organization Classification of Lung Tumors: Impact of Genetic. Clinical and Radiologic Advances Since the 2004 Classification. J Thorac Oncol 10, 1243-1260 (2015).
47. Reck, M. & Rabe, K. F. Precision Diagnosis and Treatment for Advanced Non-Small-Cell Lung Cancer. N Engl J Med 377, 849-861 (2017).
48. Ettinger, D. S. et al. NCCN Guidelines Insights: Non-Small Cell Lung Cancer, Version 1.2020. J Natl Compr Canc Netw 17, 1464-1472 (2019).
49. Wiener, R. S., Schwartz, L. M., Woloshin, S. & Welch, H. G. Population-based risk for complications after transthoracic needle lung biopsy of a pulmonary nodule: an analysis of discharge records. Ann Intern Med 155, 137-144 (2011).
50. Bubendorf, L., Lantuejoul, S., de Langen, A. J. & Thunnissen, E. Nonsmall cell lung carcinoma: diagnostic difficulties in small biopsies and cytological specimens: Number 2 in the Series “Pathology for the clinician” Edited by Peter Dorfmuller and Alberto Cavazza. Eur Respir Rev, 26 (2017).
51. McLean, A. E. B., Barnes, D. J. & Troy, L. K. Diagnosing Lung Cancer: The Complexities of Obtaining a Tissue Diagnosis in the Era of Minimally Invasive and Personalised Medicine. J Clin Med 7 (2018).
52. Reck, M. et al. Pembrolizumab versus Chemotherapy for PD-L1-Positive Non-Small-Cell Lung Cancer. N Engl J Med 375, 1823-1833 (2016).
53. Socinski, M. A. et al. Atezolizumab for First-Line Treatment of Metastatic Nonsquamous NSCLC. N Engl J Med 378, 2288-2301 (2018).
54. Gandhi, L. et al. Pembrolizumab plus Chemotherapy in Metastatic Non-Small-Cell Lung Cancer. N Engl J Med 378, 2078-2092 (2018).
55. Hellmann, M. D. et al. Nivolumab plus Ipilimumab in Lung Cancer with a High Tumor Mutational Burden. N Engl J Med 378, 2093-2104 (2018).
56. Camidge, D. R., Doebele, R. C. & Kerr, K. M. Comparing and contrasting predictive biomarkers for immunotherapy and targeted therapy of NSCLC. Nat Rev Clin Oncol 16, 341-355 (2019).
57. Nabet, B. Y. et al. Noninvasive Early Identification of Therapeutic Benefit from Immune Checkpoint Inhibition. Cell 183, 363-376 e313 (2020).
58. Menon, M. P., Pittaluga, S. & Jaffe, E. S. The histological and biological spectrum of diffuse large B-cell lymphoma in the World Health Organization classification. Cancer J 18, 411-420 (2012).
59. Sehn, L. H. et al. The revised International Prognostic Index (R-IPI) is a better predictor of outcome than the standard IP1 for patients with diffuse large B-cell lymphoma treated with R-CHOP. Blood 109, 1857-1861 (2007).
60. Alizadeh, A. A. et al. Distinct types of diffuse large B-cell lymphoma identified by gene expression profiling. Nature 403, 503-511 (2000).
61. Pasqualucci, L. et al. Analysis of the coding genome of diffuse large B-cell lymphoma. Nat Genet 43, 830-837 (2011).
62. Cottereau, A. S. et al. Molecular Profile and FDG-PET/CT Total Metabolic Tumor Volume Improve Risk Classification at Diagnosis for Patients with Diffuse Large B-Cell Lymphoma. Clin Cancer Res 22, 3801-3809 (2016).
63. Scherer, F. et al. Distinct biological subtypes and patterns of genome evolution in lymphoma revealed by circulating tumor DNA. Sci Transl Med 8, 364ra155 (2016).
64. Kurtz, D. M. et al. Circulating Tumor DNA Measurements As Early Outcome Predictors in Diffuse Large B-Cell Lymphoma. J Clin Oncol 36, 2845-2853 (2018).
65. Rosenwald, A. et al. The use of molecular profiling to predict survival after chemotherapy for diffuse large-B-cell lymphoma. N Engl J Med 346, 1937-1947 (2002).
66. Basso, K. & Dalla-Favera, R. Germinal centres and B cell lymphomagenesis. Nat Rev Immunol 15, 172-184 (2015).
67. Dunleavy, K. et al. Differential efficacy of bortezomib plus chemotherapy within molecular subtypes of diffuse large B-cell lymphoma. Blood 113, 6069-6076 (2009).
68. Thieblemont, C. et al. The germinal center/activated B-cell subclassification has a prognostic impact for response to salvage therapy in relapsed/refractory diffuse large B-cell lymphoma: a bio-CORAL study. J Clin Oncol 29.4079-4087 (2011).
69. Scott, D. W. et al. Determining cell-of-origin subtypes of diffuse large B-cell lymphoma using gene expression in formalin-fixed paraffin-embedded tissue. Blood 123, 1214-1217 (2014).
70. Nowakowski, G. S. et al. Lenalidomide combined with R-CHOP overcomes negative prognostic impact of non-germinal center B-cell phenotype in newly diagnosed diffuse large B-Cell lymphoma: a phase II study. J Clin Oncol 33, 251-257 (2015).
71. Wilson, W. H. et al. Targeting B cell receptor signaling with ibrutinib in diffuse large B cell lymphoma. Nat Med 21, 922-926 (2015).
72. Young, R. M. & Staudt, L. M. Targeting pathological B cell receptor signalling in lymphoid malignancies. Nat Rev Drug Discov 12, 229-243 (2013).
73. Lenz, G. et al. Stromal gene signatures in large-B-cell lymphomas. N Engl J Med 359, 2313-2323 (2008).
74. Zelenetz, A. D. et al. NCCN Guidelines Insights: B-Cell Lymphomas, Version 3.2019. J Natl Compr Canc Netw 17.650-661 (2019).
75. Hans, C. P. et al. Confirmation of the molecular classification of diffuse large B-cell lymphoma by immunohistochemistry using a tissue microarray. Blood 103, 275-282 (2004).
76. Lossos, I. S. et al. Prediction of survival in diffuse large-B-cell lymphoma based on the expression of six genes. N Engl J Med 350, 1828-1837 (2004).
77. Malumbres, R. et al. Paraffin-based 6-gene model predicts outcome in diffuse large B-cell lymphoma patients treated with R-CHOP. Blood 111, 5509-5514 (2008).
78. Alizadeh, A. A., Gentles, A. J., Lossos, I. S. & Levy, R. Molecular outcome prediction in diffuse large-B-cell lymphoma. N Engl J Med 360, 2794-2795 (2009).
79. Alizadeh, A. A. et al. Prediction of survival in diffuse large B-cell lymphoma based on the expression of 2 genes reflecting tumor and microenvironment. Blood 118, 1350-1358 (2011).
80. Chapuy, B. et al. Molecular subtypes of diffuse large B cell lymphoma are associated with distinct pathogenic mechanisms and outcomes. Nat Med 24, 679-690 (2018).
81. Ennishi, D. et al. Double-Hit Gene Expression Signature Defines a Distinct Subgroup of Germinal Center B-Cell-Like Diffuse Large B-Cell Lymphoma. J Clin Oncol 37, 190-201 (2019).
82. Gentles, A. J. & Alizadeh, A. A. A few good genes: simple, biologically motivated signatures for cancer prognosis. Cell Cycle 10, 3615-3616 (2011).
83. Chambers, J. & Rabbitts, T. H. LMO2 at 25 years: a paradigm of chromosomal translocation proteins. Open Biol 5, 150062 (2015).
84. Royer-Pokora, B. et al. The TTG-2/RBTN2 T cell oncogene encodes two alternative transcripts from two promoters: the distal promoter is removed by most 11p13 translocations in acute T cell leukaemia's (T-ALL). Oncogene 10, 1353-1360 (1995).
85. Oram, S. H. et al. A previously unrecognized promoter of LMO2 forms part of a transcriptional regulatory circuit mediating LMO2 expression in a subset of T-acute lymphoblastic leukaemia patients. Oncogene 29, 5796-5808 (2010).
86. Boehm, T. et al. An unusual structure of a putative T cell oncogene which allows production of similar proteins from distinct mRNAs. EMBO J 9, 857-868 (1990).
87. Smale, S. T. & Kadonaga, J. T. The RNA polymerase II core promoter. Annu Rev Biochem 72, 449-479 (2003).
88. Bernstein, B. E. et al. Genomic maps and comparative analysis of histone modifications in human and mouse. Cell 120, 169-181 (2005).
89. Wong, I. H. et al. Detection of aberrant p16 methylation in the plasma and serum of liver cancer patients. Cancer Res 59, 71-73 (1999).
90. Chim, S. S. et al. Detection of the placental epigenetic signature of the maspin gene in maternal plasma. Proc Natl Acad Sci USA 102, 14753-14758 (2005).
91. Fernandez, A. F. et al. A DNA methylation fingerprint of 1628 human samples. Genome Res 22, 407-419 (2012).
92. Houseman, E. A. et al. DNA methylation arrays as surrogate measures of cell mixture distribution. BMC Bioinformatics 13, 86 (2012).
93. Chan, K. C. et al. Noninvasive detection of cancer-associated genome-wide hypomethylation and copy number aberrations by plasma DNA bisulfite sequencing. Proc Natl Acad Sci USA 110, 18761-18768 (2013).
94. Lun, F. M. et al. Noninvasive prenatal methylomic analysis by genomewide bisulfite sequencing of maternal plasma DNA. Clin Chen 59, 1583-1594 (2013).
95. Ou, X. et al. Epigenome-wide DNA methylation assay reveals placental epigenetic markers for noninvasive fetal single-nucleotide polymorphism genotyping in maternal plasma. Transfusion 54, 2523-2533 (2014).
96. Jensen, T. J. et al. Whole genome bisulfite sequencing of cell-free DNA and its cellular contributors uncovers placenta hypomethylated domains. Genome Biol 16, 78 (2015).
97. Roadmap Epigenomics, C. et al. Integrative analysis of 111 reference human epigenomes. Nature 518, 317-330 (2015).
98. Visel, A. et al. ChIP-seq accurately predicts tissue-specific activity of enhancers. Nature 457, 854-858 (2009).
99. Koh, W. et al. Noninvasive in vivo monitoring of tissue-specific global gene expression in humans. Proc Natl Acad Sci USA 111, 7361-7366 (2014).
100. Srinivasan, S. et al. Small RNA Sequencing across Diverse Biofluids Identifies Optimal Methods for ex RNA Isolation. Cell 177, 446-462 e416 (2019).
101. Ibarra, A. et al. Non-invasive characterization of human bone marrow stimulation and reconstitution by cell-free messenger RNA sequencing. Nat Commun 11, 400 (2020).
102. Zhou, Z. et al. Extracellular RNA in a single droplet of human serum reflects physiologic and disease states. Proc Natl Acad Sci USA 116, 19200-19208 (2019).
103. Verwilt, J. et al. When DNA gets in the way: A cautionary note for DNA contamination in extracellular RNA-seq studies. Proc Natl Acad Sci USA 117, 18934-18936 (2020).
104. Gentles, A. J. et al. The prognostic landscape of genes and infiltrating immune cells across human cancers. Nat Med 21.938-945 (2015).
105. Binkley, M. S. et al. KEAP1/NFE2L2 Mutations Predict Lung Cancer Radiation Resistance That Can Be Targeted by Glutaminase Inhibition. Cancer Discov 10, 1826-1841 (2020).
106. Alig S. et al. Short Diagnosis-to-Treatment Interval is associated with increased tumor burden measured by circulating tumor DNA and metabolic tumor volume in Diffuse Large B-cell Lymphoma. Journal of Clinical Oncology in press (2021).
107. Patro, R., Duggal, G., Love, M. I., Irizarry, R. A. & Kingsford, C. Salmon provides fast and bias-aware quantification of transcript expression. Nat Methods 14, 417-419 (2017).
108. Chen, S., Zhou, Y., Chen, Y. & Gu, J. fastp: an ultra-fast all-in-one FASTQ preprocessor. Bioinformatics 34, i884-i890 (2018).
109. George, J. et al. Comprehensive genomic profiles of small cell lung cancer. Nature 524, 47-53 (2015).
110. U, M., Talevich, E., Katiyar, S., Rasheed, K. & Kannan, N. Prediction and prioritization of rare oncogenic mutations in the cancer Kinome using novel features and multiple classifiers. PLoS Comput Biol 10, e1003545 (2014).
111. Venkatraman, E. S. & Olshen, A. B. A faster circular binary segmentation algorithm for the analysis of array CGH data. Bioinformatics 23, 657-663 (2007).
112. Newman, A. M. et al. Determining cell type abundance and expression from bulk tissues with digital cytometry. Nat Biotechnol 37, 773-782 (2019).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE1
Transcription Start Sites for Interrogation to Detect Lymphoid Diseases

| CHR | TSS | Start | Stop | Gene-Symbol | Strand | CHR | TSS | Start | Stop | Gene-Symbol | Strand |
|---|---|---|---|---|---|---|---|---|---|---|---|
| chr1 | 154580682 | 154579932 | 154581432 | ADAR | −1 | chr2 | 202645912 | 202645162 | 202646662 | ALS2 | −1 |
| chr1 | 159046691 | 159045941 | 159047441 | AIM2 | −1 | chr2 | 112642267 | 112641517 | 112643017 | ANAPC1 | −1 |
| chr1 | 197115824 | 197115074 | 197116574 | ASPM | −1 | chr2 | 69240310 | 69239560 | 69241060 | ANTXR1 | 1 |
| chr1 | 212782012 | 212781262 | 212782762 | ATF3 | 1 | chr2 | 68961967 | 68961217 | 68962717 | ARHGAP25 | 1 |
| chr1 | 169074935 | 169074185 | 169075685 | ATP1B1 | 1 | chr2 | 219081933 | 219081183 | 219082683 | ARPC2 | 1 |
| chr1 | 154300276 | 154299526 | 154301026 | ATP8B2 | 1 | chr2 | 9346894 | 9346144 | 9347644 | ASAP2 | 1 |
| chr1 | 154298035 | 154297285 | 154298785 | ATP8B2 | 1 | chr2 | 176049335 | 176048585 | 176050085 | ATP5MC3 | −1 |
| chr1 | 212873327 | 212872577 | 212874077 | BATF3 | −1 | chr2 | 27434895 | 27434145 | 27435645 | ATRAID | 1 |
| chr1 | 85742773 | 85742023 | 85743523 | BCL10 | −1 | chr2 | 60780633 | 60779883 | 60781383 | BCL11A | −1 |
| chr1 | 220263804 | 220263054 | 220264554 | BPNT1 | −1 | chr2 | 111435691 | 111434941 | 111436441 | BUB1 | −1 |
| chr1 | 52521797 | 52521047 | 52522547 | BTF3L4 | 1 | chr2 | 47403740 | 47402990 | 47404490 | CALM2 | −1 |
| chr1 | 203274664 | 203273914 | 203275414 | BTG2 | 1 | chr2 | 202047604 | 202046854 | 202048354 | CASP10 | 1 |
| chr1 | 22962999 | 22962249 | 22963749 | C1QA | 1 | chr2 | 202122759 | 202122009 | 202123509 | CASP8 | 1 |
| chr1 | 22979682 | 22978932 | 22980432 | C1QB | 1 | chr2 | 202098165 | 202097415 | 202098915 | CASP8 | 1 |
| chr1 | 22970126 | 22969376 | 22970876 | C1QC | 1 | chr2 | 202125222 | 202124472 | 202125972 | CASP8 | 1 |
| chr1 | 156308256 | 156307506 | 156309006 | CCT3 | −1 | chr2 | 55646749 | 55645999 | 55647499 | CCDC88A | −1 |
| chr1 | 145706861 | 145706111 | 145707611 | CD160 | −1 | chr2 | 228678570 | 228677820 | 228679320 | CCL20 | 1 |
| chr1 | 158223927 | 158223177 | 158224677 | CD1A | 1 | chr2 | 204571271 | 204570521 | 204572021 | CD28 | 1 |
| chr1 | 158301321 | 158300571 | 158302071 | CD1B | −1 | chr2 | 87035519 | 87034769 | 87036269 | CD8A | −1 |
| chr1 | 158259576 | 158258826 | 158260326 | CD1C | 1 | chr2 | 87018837 | 87018087 | 87019587 | CD8A | −1 |
| chr1 | 158323540 | 158322790 | 158324290 | CD1E | 1 | chr2 | 87089038 | 87088288 | 87089788 | CD8B | −1 |
| chr1 | 117297007 | 117296257 | 117297757 | CD2 | 1 | chr2 | 174219589 | 174218839 | 174220339 | CDCA7 | 1 |
| chr1 | 160832550 | 160831800 | 160833300 | CD244 | −1 | chr2 | 27008865 | 27008115 | 27009615 | CENPA | 1 |
| chr1 | 167487775 | 167487025 | 167488525 | CD247 | −1 | chr2 | 201980827 | 201980077 | 201981577 | CFLAR | 1 |
| chr1 | 208084747 | 208083997 | 208085497 | CD34 | −1 | chr2 | 175869954 | 175869204 | 175870704 | CHN1 | −1 |
| chr1 | 207925402 | 207924652 | 207926152 | CD46 | 1 | chr2 | 220408509 | 220407759 | 220409259 | CHPF | −1 |
| chr1 | 26644448 | 26643698 | 26645198 | CD52 | 1 | chr2 | 165697922 | 165697172 | 165698672 | COBLL1 | −1 |
| chr1 | 117113661 | 117112911 | 117114411 | CD58 | −1 | chr2 | 189839046 | 189838296 | 189839796 | COL3A1 | 1 |
| chr1 | 43824684 | 43823934 | 43825434 | CDC20 | 1 | chr2 | 36583069 | 36582319 | 36583819 | CRIM1 | 1 |
| chr1 | 91966665 | 91965915 | 91967415 | CDC7 | 1 | chr2 | 46844290 | 46843540 | 46845040 | CRIPT | 1 |
| chr1 | 91966403 | 91965653 | 91967153 | CDC7 | 1 | chr2 | 204732509 | 204731759 | 204733259 | CTLA4 | 1 |
| chr1 | 38158090 | 38157340 | 38158840 | CDCA8 | 1 | chr2 | 136873813 | 136873063 | 136874563 | CXCR4 | −1 |
| chr1 | 214776538 | 214775788 | 214777288 | CENPF | 1 | chr2 | 136875725 | 136874975 | 136876475 | CXCR4 | −1 |
| chr1 | 203155877 | 203155127 | 203156627 | CHI3L1 | −1 | chr2 | 219646479 | 219645729 | 219647229 | CYP27A1 | 1 |
| chr1 | 111743393 | 111742643 | 111744143 | CHI3L2 | 1 | chr2 | 120124829 | 120124079 | 120125579 | DBI | 1 |
| chr1 | 203198799 | 203198049 | 203199549 | CHIT1 | −1 | chr2 | 120124503 | 120123753 | 120125253 | DBI | 1 |
| chr1 | 154947175 | 154946425 | 154947925 | CKS1B | 1 | chr2 | 120124552 | 120123802 | 120125302 | DBI | 1 |
| chr1 | 25071848 | 25071098 | 25072598 | CLIC4 | 1 | chr2 | 120125250 | 120124500 | 120126000 | DBI | 1 |
| chr1 | 26503894 | 26503144 | 26504644 | CNKSR1 | 1 | chr2 | 169921299 | 169920549 | 169922049 | DHRS9 | 1 |
| chr1 | 95392834 | 95392084 | 95393584 | CNN3 | −1 | chr2 | 169923544 | 169922794 | 169924294 | DHRS9 | 1 |
| chr1 | 24285549 | 24284799 | 24286299 | CNR2 | −1 | chr2 | 169929084 | 169928334 | 169929834 | DHRS9 | 1 |
| chr1 | 32169920 | 32169170 | 32170670 | COL1GA1 | −1 | chr2 | 169937616 | 169936866 | 169938366 | DHRS9 | 1 |
| chr1 | 207627575 | 207626825 | 207628325 | CR2 | 1 | chr2 | 84743579 | 84742829 | 84744329 | DNAH6 | 1 |
| chr1 | 115292811 | 115292061 | 115293561 | CSDE1 | −1 | chr2 | 225907159 | 225906409 | 225907909 | DOCK10 | −1 |
| chr1 | 110453255 | 110452505 | 110454005 | CSF1 | 1 | chr2 | 32264881 | 32264131 | 32265631 | DPY30 | −1 |
| chr1 | 85040147 | 85039397 | 85040897 | CTBS | −1 | chr2 | 25896503 | 25895753 | 25897253 | DTNB | −1 |
| chr1 | 70876955 | 70876205 | 70877705 | CTH | 1 | chr2 | 207024329 | 207023579 | 207025079 | EEF1B2 | 1 |
| chr1 | 41445007 | 41444257 | 41445757 | CTPS1 | 1 | chr2 | 31457203 | 31456453 | 31457953 | EHD3 | 1 |
| chr1 | 150738433 | 150737683 | 150739183 | CTSS | −1 | chr2 | 54951679 | 54950929 | 54952429 | EML6 | 1 |
| chr1 | 47533160 | 47532410 | 47533910 | CYP4Z1 | 1 | chr2 | 46524541 | 46523791 | 46525291 | EPAS1 | 1 |
| chr1 | 111743311 | 111742561 | 111744061 | DENND2D | −1 | chr2 | 75788092 | 75787342 | 75788842 | FAM176A | −1 |
| chr1 | 12677737 | 12676987 | 12678487 | DHRS3 | −1 | chr2 | 75796848 | 75796098 | 75797598 | FAM176A | −1 |
| chr1 | 93427057 | 93426307 | 93427807 | DIPK1A | −1 | chr2 | 216300895 | 216300145 | 216301645 | FN1 | −1 |
| chr1 | 98386579 | 98385829 | 98387329 | DPYD | −1 | chr2 | 31361013 | 31360263 | 31361763 | GALNT14 | −1 |
| chr1 | 23857712 | 23856962 | 23858462 | E2F2 | −1 | chr2 | 85921414 | 85920664 | 85922164 | GNLY | 1 |
| chr1 | 43638241 | 43637491 | 43638991 | EBNA1BP2 | −1 | chr2 | 234763212 | 234762462 | 234763962 | HJURP | −1 |
| chr1 | 43637986 | 43637236 | 43638736 | EBNA1BP2 | −1 | chr2 | 176994468 | 176993718 | 176995218 | HOXD8 | 1 |
| chr1 | 225840844 | 225840094 | 225841594 | ENAH | −1 | chr2 | 176994421 | 176993671 | 176995171 | HOXD8 | 1 |
| chr1 | 8938749 | 8937999 | 8939499 | ENO1 | −1 | chr2 | 198364771 | 198364021 | 198365521 | HSPD1 | −1 |
| chr1 | 16482582 | 16481832 | 16483332 | EPHA2 | −1 | chr2 | 198364718 | 198363968 | 198365468 | HSPE1 | 1 |
| chr1 | 242011482 | 242010732 | 242012232 | EXO1 | 1 | chr2 | 204801471 | 204800721 | 204802221 | ICOS | 1 |
| chr1 | 242012032 | 242011282 | 242012782 | EXO1 | 1 | chr2 | 8818975 | 8818225 | 8819725 | ID2 | 1 |
| chr1 | 160991138 | 160990388 | 160991888 | F11R | −1 | chr2 | 163175213 | 163174463 | 163175963 | IFIH1 | −1 |
| chr1 | 172628158 | 172627408 | 172628908 | FASLG | 1 | chr2 | 214015058 | 214014308 | 214015808 | IKZF2 | −1 |
| chr1 | 16090994 | 16090244 | 16091744 | FBLIM1 | 1 | chr2 | 214016333 | 214015583 | 214017083 | IKZF2 | −1 |
| chr1 | 16085254 | 16084504 | 16086004 | FBLIM1 | 1 | chr2 | 102759246 | 102758496 | 102759996 | IL1R1 | 1 |
| chr1 | 16091458 | 16090708 | 16092208 | FBLIM1 | 1 | chr2 | 102608306 | 102607556 | 102609056 | IL1R2 | 1 |
| chr1 | 207143945 | 207143195 | 207144695 | FCAMR | −1 | chr2 | 102615423 | 102614673 | 102616173 | IL1R2 | 1 |
| chr1 | 161185087 | 161184337 | 161185837 | FCER1G | 1 | chr2 | 191208196 | 191207446 | 191208946 | INPP1 | 1 |
| chr1 | 149754246 | 149753496 | 149754996 | FCGR1A | 1 | chr2 | 182321952 | 182321202 | 182322702 | ITGA4 | 1 |
| chr1 | 120935937 | 120935187 | 120936687 | FCGR1B | −1 | chr2 | 187454792 | 187454042 | 187455542 | ITGAV | 1 |
| chr1 | 161632951 | 161632201 | 161633701 | FCGR2B | 1 | chr2 | 187464931 | 187464181 | 187465681 | ITGAV | 1 |
| chr1 | 161519818 | 161519068 | 161520568 | FCGR3A | −1 | chr2 | 47798078 | 47797328 | 47798828 | KCNK12 | −1 |
| chr1 | 161520413 | 161519663 | 161521163 | FCGR3A | −1 | chr2 | 170590356 | 170589606 | 170591106 | KLHL23 | 1 |
| chr1 | 161600996 | 161600246 | 161601746 | FCGR3B | −1 | chr2 | 20251789 | 20251039 | 20252539 | LAPTM4A | −1 |
| chr1 | 207095343 | 207094593 | 207096093 | FCMR | −1 | chr2 | 109271493 | 109270743 | 109272243 | LIMS1 | 1 |
| chr1 | 157789895 | 157789145 | 157790645 | FCRL1 | −1 | chr2 | 142889270 | 142888520 | 142890020 | LRP1B | −1 |
| chr1 | 157746912 | 157746162 | 157747662 | FCRL2 | −1 | chr2 | 33172039 | 33171289 | 33172789 | LTBP1 | 1 |
| chr1 | 157670647 | 157669897 | 157671397 | FCRL3 | −1 | chr2 | 160761193 | 160760443 | 160761943 | LY75 | −1 |
| chr1 | 157567870 | 157567120 | 157568620 | FCRL4 | −1 | chr2 | 149894981 | 149894231 | 149895731 | LYPD6B | 1 |
| chr1 | 157522310 | 157521560 | 157523060 | FCRL5 | −1 | chr2 | 95691429 | 95690679 | 95692179 | MAL | 1 |
| chr1 | 161676762 | 161676012 | 161677512 | FCRLA | 1 | chr2 | 119699742 | 119698992 | 119700492 | MARCO | 1 |
| chr1 | 155278707 | 155277957 | 155279457 | FDPS | 1 | chr2 | 27994584 | 27993834 | 27995334 | MRPL33 | 1 |
| chr1 | 155278538 | 155277788 | 155279288 | FDPS | 1 | chr2 | 48010221 | 48009471 | 48010971 | MSH6 | 1 |
| chr1 | 27961788 | 27961038 | 27962538 | FGR | −1 | chr2 | 241075764 | 241075014 | 241076514 | MYEOV2 | −1 |
| chr1 | 203320617 | 203319867 | 203321367 | FMOD | −1 | chr2 | 241075747 | 241074997 | 241076497 | MYEOV2 | −1 |
| chr1 | 93913688 | 93912938 | 93914438 | FNBP1L | 1 | chr2 | 97001525 | 97000775 | 97002275 | NCAPH | 1 |
| chr1 | 68150744 | 68149994 | 68151494 | GADD45A | 1 | chr2 | 232329305 | 232328555 | 232330055 | NCL | −1 |
| chr1 | 173837125 | 173836375 | 173837875 | GAS5 | −1 | chr2 | 24714801 | 24714051 | 24715551 | NCOA1 | 1 |
| chr1 | 89531043 | 89530293 | 89531793 | GBP1 | −1 | chr2 | 157189228 | 157188478 | 157189978 | NR4A2 | −1 |
| chr1 | 89591797 | 89591047 | 89592547 | GBP2 | −1 | chr2 | 113479063 | 113478313 | 113479813 | NT5DC4 | 1 |
| chr1 | 35258599 | 35257849 | 35259349 | GJA4 | 1 | chr2 | 10588630 | 10587880 | 10589380 | ODC1 | −1 |
| chr1 | 182361341 | 182360591 | 182362091 | GLUL | −1 | chr2 | 242801060 | 242800310 | 242801810 | PDCD1 | −1 |
| chr1 | 182360539 | 182359789 | 182361289 | GLUL | −1 | chr2 | 64371588 | 64370838 | 64372338 | PELI1 | −1 |
| chr1 | 236305832 | 236305082 | 236306582 | GPR137B | 1 | chr2 | 68592305 | 68591555 | 68593055 | PLEK | 1 |
| chr1 | 228327991 | 228327241 | 228328741 | GUK1 | 1 | chr2 | 43864412 | 43863662 | 43865162 | PLEKHH2 | 1 |
| chr1 | 149858232 | 149857482 | 149858982 | H2BC21 | −1 | chr2 | 136289083 | 136288333 | 136289833 | R3HDM1 | 1 |
| chr1 | 226251678 | 226250928 | 226252428 | H3-3A | 1 | chr2 | 61108656 | 61107906 | 61109406 | REL | 1 |
| chr1 | 32757687 | 32756937 | 32758437 | HDAC1 | 1 | chr2 | 152266455 | 152265705 | 152267205 | RIF1 | 1 |
| chr1 | 87380357 | 87379607 | 87381107 | HS2ST1 | 1 | chr2 | 217362912 | 217362162 | 217363662 | RPL37A | 1 |
| chr1 | 23886285 | 23885535 | 23887035 | JD3 | −1 | chr2 | 3622795 | 3622045 | 3623545 | RPS7 | 1 |
| chr1 | 158979682 | 158978932 | 158980432 | IFI16 | 1 | chr2 | 10262455 | 10261705 | 10263205 | RRM2 | 1 |
| chr1 | 117210314 | 117209564 | 117211064 | IGSF3 | −1 | chr2 | 20425194 | 20424444 | 20425944 | SDC1 | −1 |
| chr1 | 67773047 | 67772297 | 67773797 | IL12RB2 | 1 | chr2 | 20424927 | 20424177 | 20425677 | SDC1 | −1 |
| chr1 | 156542332 | 156541582 | 156543082 | IQGAP3 | −1 | chr2 | 26568982 | 26568232 | 26569732 | SELENOI | 1 |
| chr1 | 948803 | 948053 | 949553 | ISG15 | 1 | chr2 | 97535708 | 97534958 | 97536458 | SEMA4C | −1 |
| chr1 | 226926864 | 226926114 | 226927614 | ITPKB | −1 | chr2 | 110371783 | 110371033 | 110372533 | SEPTIN10 | −1 |
| chr1 | 59249785 | 59249035 | 59250535 | JUN | −1 | chr2 | 85895864 | 85895114 | 85896614 | SFTPB | −1 |
| chr1 | 175161890 | 175161140 | 175162640 | KIAA0040 | −1 | chr2 | 201390880 | 201390130 | 201391630 | SGO2 | 1 |
| chr1 | 200589862 | 200589112 | 200590612 | KIF14 | −1 | chr2 | 223289236 | 223288486 | 223289986 | SGPP2 | 1 |
| chr1 | 45205492 | 45204742 | 45206242 | KIF2C | 1 | chr2 | 264065 | 263315 | 264815 | SH3YL1 | −1 |
| chr1 | 6662929 | 6662179 | 6663679 | KLHL21 | −1 | chr2 | 65216535 | 65215785 | 65217285 | SUC1A4 | 1 |
| chr1 | 31230667 | 31229917 | 31231417 | LAPTM5 | −1 | chr2 | 220034754 | 220034004 | 220035504 | SLC23A3 | −1 |
| chr1 | 32716840 | 32716090 | 32717590 | LCK | 1 | chr2 | 190445613 | 190444863 | 190446363 | SLC40A1 | −1 |
| chr1 | 32739711 | 32738961 | 32740461 | LCK | 1 | chr2 | 162480936 | 162480186 | 162481686 | SLC4A10 | 1 |
| chr1 | 202163029 | 202162279 | 202163779 | LGR6 | 1 | chr2 | 17935070 | 17934320 | 17935820 | SMC6 | −1 |
| chr1 | 153232176 | 153231426 | 153232926 | LORICR1N | 1 | chr2 | 70520903 | 70520153 | 70521653 | SNRPG | −1 |
| chr1 | 90098631 | 90097881 | 90099381 | LRRC8C | 1 | chr2 | 39348192 | 39347442 | 39348942 | SOS1 | −1 |
| chr1 | 32801980 | 32801230 | 32802730 | MARCKSL1 | −1 | chr2 | 5832799 | 5832049 | 5833549 | SOX11 | 1 |
| chr1 | 150552066 | 150551316 | 150552816 | MCL1 | −1 | chr2 | 214149113 | 214148363 | 214149863 | SPAG16 | 1 |
| chr1 | 17307330 | 17306580 | 17308080 | MFAP2 | −1 | chr2 | 65659311 | 65658561 | 65660061 | SPRED2 | −1 |
| chr1 | 158801107 | 158800357 | 158801857 | MNDA | 1 | chr2 | 191878976 | 191878226 | 191879726 | STAT1 | −1 |
| chr1 | 47080805 | 47080055 | 47081555 | MOB3C | −1 | chr2 | 192015986 | 192015236 | 192016736 | STAT4 | −1 |
| chr1 | 47082563 | 47081813 | 47083313 | MOB3C | −1 | chr2 | 192016322 | 192015572 | 192017072 | STAT4 | −1 |
| chr1 | 161279762 | 161279012 | 161280512 | MPZ | −1 | chr2 | 197036299 | 197035549 | 197037049 | STK17B | −1 |
| chr1 | 155579978 | 155579228 | 155580728 | MSTO2P | 1 | chr2 | 105946491 | 105945741 | 105947241 | TGFBRAP1 | −1 |
| chr1 | 155715558 | 155714808 | 155716308 | MSTO2P | 1 | chr2 | 70475773 | 70475023 | 70476523 | TIA1 | −1 |
| chr1 | 46049732 | 46048982 | 46050482 | NASP | 1 | chr2 | 220118754 | 220118004 | 220119504 | TUBA4A | −1 |
| chr1 | 145293371 | 145292621 | 145294121 | NBPF10 | 1 | chr2 | 136499189 | 136498439 | 136499939 | UBXN4 | 1 |
| chr1 | 16767231 | 16766481 | 16767981 | NECAP2 | 1 | chr2 | 224810104 | 224809354 | 224810854 | WDFY1 | −1 |
| chr1 | 184943682 | 184942932 | 184944432 | NIBAN1 | −1 | chr2 | 30369807 | 30369057 | 30370557 | YPEL5 | 1 |
| chr1 | 120612240 | 120611490 | 120612990 | NOTCH2 | −1 | chr2 | 98330023 | 98329273 | 98330773 | ZAP70 | 1 |
| chr1 | 145589439 | 145588689 | 145590189 | NUDT17 | −1 | chr2 | 98350868 | 98350118 | 98351618 | ZAP70 | 1 |
| chr1 | 241803663 | 241802913 | 241804413 | OPN3 | −1 | chr2 | 185463093 | 185462343 | 185463843 | ZNF804A | 1 |
| chr1 | 66258197 | 66257447 | 66258947 | PDE4B | 1 | chr20 | 32891154 | 32890404 | 32891904 | AHCY | −1 |
| chr1 | 66258855 | 66258105 | 66259605 | PDE4B | 1 | chr20 | 30946155 | 30945405 | 30946905 | ASXL1 | 1 |
| chr1 | 66458389 | 66457639 | 66459139 | PDE4B | 1 | chr20 | 54967351 | 54966601 | 54968101 | AURKA | −1 |
| chr1 | 66797792 | 66797042 | 66798542 | PDE4B | 1 | chr20 | 49411431 | 49410681 | 49412181 | BCAS4 | 1 |
| chr1 | 144995022 | 144994272 | 144995772 | PDE4DIP | −1 | chr20 | 30310701 | 30309951 | 30311451 | BCL2L1 | −1 |
| chr1 | 144932032 | 144931282 | 144932782 | PDE4DIP | −1 | chr20 | 55841684 | 55840934 | 55842434 | BMP7 | −1 |
| chr1 | 144995033 | 144994283 | 144995783 | PDE4DIP | −1 | chr20 | 44746911 | 44746161 | 44747661 | CD40 | 1 |
| chr1 | 144995082 | 144994332 | 144995832 | PDE4DIP | −1 | chr20 | 23066977 | 23066227 | 23067727 | CD93 | −1 |
| chr1 | 144997111 | 144996361 | 144997861 | PDE4DIP | −1 | chr20 | 61448414 | 61447664 | 61449164 | COL9A3 | 1 |
| chr1 | 145039992 | 145039242 | 145040742 | PDE4DIP | −1 | chr20 | 524465 | 523715 | 525215 | CSNK2A1 | −1 |
| chr1 | 145076186 | 145075436 | 145076936 | PDE4DIP | −1 | chr20 | 24929866 | 24929116 | 24930616 | CST7 | 1 |
| chr1 | 13909960 | 13909210 | 13910710 | PDPN | 1 | chr20 | 30458550 | 30457800 | 30459300 | DUSP15 | −1 |
| chr1 | 160175127 | 160174377 | 160175877 | PEA15 | 1 | chr20 | 37554955 | 37554205 | 37555705 | FAM83D | 1 |
| chr1 | 207119811 | 207119061 | 207120561 | PIGR | −1 | chr20 | 42355638 | 42354888 | 42356388 | GTSF1L | −1 |
| chr1 | 9711803 | 9711053 | 9712553 | PIK3CD | 1 | chr20 | 30640064 | 30639314 | 30640814 | HCK | 1 |
| chr1 | 6557156 | 6556406 | 6557906 | PLEKHG5 | −1 | chr20 | 13976015 | 13975265 | 13976765 | MACROD2 | 1 |
| chr1 | 6545529 | 6544779 | 6546279 | PLEKHG5 | −1 | chr20 | 39317880 | 39317130 | 39318630 | MAFB | −1 |
| chr1 | 6546014 | 6545264 | 6546764 | PLEKHG5 | −1 | chr20 | 44637547 | 44636797 | 44638297 | MMP9 | 1 |
| chr1 | 6550640 | 6549890 | 6551390 | PLEKHG5 | −1 | chr20 | 25566153 | 25565403 | 25566903 | NINL | −1 |
| chr1 | 6550654 | 6549904 | 6551404 | PLEKHG5 | −1 | chr20 | 31071274 | 31070524 | 31072024 | NOL4L | −1 |
| chr1 | 6551761 | 6551011 | 6552511 | PLEKHG5 | −1 | chr20 | 2632791 | 2632041 | 2633541 | NOP56 | 1 |
| chr1 | 6557484 | 6556734 | 6558234 | PLEKHG5 | −1 | chr20 | 23331373 | 23330623 | 23332123 | NXT1 | 1 |
| chr1 | 6580121 | 6579371 | 6580871 | PLEKHG5 | −1 | chr20 | 56284958 | 56284208 | 56285708 | PMEPA1 | −1 |
| chr1 | 173446405 | 173445655 | 173447155 | PRDX6 | 1 | chr20 | 49126891 | 49126141 | 49127641 | PTPN1 | 1 |
| chr1 | 84609954 | 84609204 | 84610704 | PRKACB | 1 | chr20 | 55966463 | 55965713 | 55967213 | RBM38 | 1 |
| chr1 | 84543744 | 84542994 | 84544494 | PRKACB | 1 | chr20 | 19867165 | 19866415 | 19867915 | RIN2 | 1 |
| chr1 | 84630064 | 84629314 | 84630814 | PRKACB | 1 | chr20 | 19870209 | 19869459 | 19870959 | RIN2 | 1 |
| chr1 | 84630648 | 84629898 | 84631398 | PRKACB | 1 | chr20 | 34287194 | 34286444 | 34287944 | ROMO1 | 1 |
| chr1 | 84647342 | 84646592 | 84648092 | PRKACB | 1 | chr20 | 35580246 | 35579496 | 35580996 | SAMHD1 | −1 |
| chr1 | 151227179 | 151226429 | 151227929 | PSMD4 | 1 | chr20 | 1875942 | 1875192 | 1876692 | SIRPA | 1 |
| chr1 | 117452679 | 117451929 | 117453429 | PTGFRN | 1 | chr20 | 35274619 | 35273869 | 35275369 | SLA2 | −1 |
| chr1 | 198608170 | 198607420 | 198608920 | PTPRC | 1 | chr20 | 4129426 | 4128676 | 4130176 | SMOX | 1 |
| chr1 | 43996547 | 43995797 | 43997297 | PTPRF | 1 | chr20 | 17949623 | 17948873 | 17950373 | SNX5 | −1 |
| chr1 | 29563028 | 29562278 | 29563778 | PTPRU | 1 | chr20 | 31592239 | 31591489 | 31592989 | SUN5 | −1 |
| chr1 | 180123969 | 180123219 | 180124719 | QSOX1 | 1 | chr20 | 39657458 | 39656708 | 39658208 | TOP1 | 1 |
| chr1 | 205744574 | 205743824 | 205745324 | RAB29\|RAB7L | −1 | chr20 | 30327074 | 30326324 | 30327824 | TPX2 | 1 |
| chr1 | 178694300 | 178693550 | 178695050 | RALGPS2 | 1 | chr20 | 44259907 | 44259157 | 44260657 | WFDC9 | −1 |
| chr1 | 65210778 | 65210028 | 65211528 | RAVER2 | 1 | chr21 | 45705763 | 45705013 | 45706513 | AIRE | 1 |
| chr1 | 28844769 | 28844019 | 28845519 | RCC1 | 1 | chr21 | 27542972 | 27542222 | 27543722 | APP | −1 |
| chr1 | 167599330 | 167598580 | 167600080 | RCSD1 | 1 | chr21 | 27107344 | 27106594 | 27108094 | ATP5J | −1 |
| chr1 | 192544857 | 192544107 | 192545607 | RGS1 | 1 | chr21 | 47401651 | 47400901 | 47402401 | COL6A1 | 1 |
| chr1 | 192605275 | 192604525 | 192606025 | RGS13 | 1 | chr21 | 40033618 | 40032868 | 40034368 | ERG | −1 |
| chr1 | 192778171 | 192777421 | 192778921 | RGS2 | 1 | chr21 | 39870428 | 39869678 | 39871178 | ERG | −1 |
| chr1 | 64239714 | 64238964 | 64240464 | ROR1 | 1 | chr21 | 39956869 | 39956119 | 39957619 | ERG | −1 |
| chr1 | 151804348 | 151803598 | 151805098 | RORC | −1 | chr21 | 40032591 | 40031841 | 40033341 | ERG | −1 |
| chr1 | 151798553 | 151797803 | 151799303 | RORC | −1 | chr21 | 40033704 | 40032954 | 40034454 | ERG | −1 |
| chr1 | 45241109 | 45240359 | 45241859 | RPS8 | 1 | chr21 | 40177231 | 40176481 | 40177981 | ETS2 | 1 |
| chr1 | 151966866 | 151966116 | 151967616 | S100A10 | −1 | chr21 | 40177754 | 40177004 | 40178504 | ETS2 | 1 |
| chr1 | 152009511 | 152008761 | 152010261 | S100A11 | −1 | chr21 | 45660828 | 45660078 | 45661578 | ICOSLG | −1 |
| chr1 | 153518361 | 153517611 | 153519111 | S100A4 | −1 | chr21 | 46348788 | 46348038 | 46349538 | ITGB2 | −1 |
| chr1 | 1215816 | 1215066 | 1216566 | SCNN1D | 1 | chr21 | 35883613 | 35882863 | 35884363 | KCNE1 | −1 |
| chr1 | 243419358 | 243418608 | 243420108 | SDCCAG8 | 1 | chr21 | 35828107 | 35827357 | 35828857 | KCNE1 | −1 |
| chr1 | 169680839 | 169680089 | 169681589 | SELL | −1 | chr21 | 35831902 | 35831152 | 35832652 | KCNE1 | −1 |
| chr1 | 169677997 | 169677247 | 169678747 | SELL | −1 | chr21 | 35832236 | 35831486 | 35832986 | KCNE1 | −1 |
| chr1 | 156123318 | 156122568 | 156124068 | SEMA4A | 1 | chr21 | 35883637 | 35882887 | 35884387 | KCNE1 | −1 |
| chr1 | 156119734 | 156118984 | 156120484 | SEMA4A | 1 | chr21 | 35884573 | 35883823 | 35885323 | KCNE1 | −1 |
| chr1 | 156123368 | 156122618 | 156124118 | SEMA4A | 1 | chr21 | 26934456 | 26933706 | 26935206 | MIR155HG | 1 |
| chr1 | 154943223 | 154942473 | 154943973 | SHC1 | −1 | chr21 | 16437321 | 16436571 | 16438071 | NRIP1 | −1 |
| chr1 | 154946959 | 154946209 | 154947709 | SHC1 | −1 | chr21 | 45138993 | 45138243 | 45139743 | PDXK | 1 |
| chr1 | 53099016 | 53098266 | 53099766 | SHISAL2A | 1 | chr21 | 45719934 | 45719184 | 45720684 | PFKL | 1 |
| chr1 | 160617085 | 160616335 | 160617835 | SLAMF1 | −1 | chr21 | 43299591 | 43298841 | 43300341 | PRDM15 | −1 |
| chr1 | 160493043 | 160492293 | 160493793 | SLAMF6 | −1 | chr21 | 45079429 | 45078679 | 45080179 | RRP1B | 1 |
| chr1 | 160709055 | 160708305 | 160709805 | SLAMF7 | 1 | chr21 | 15955723 | 15954973 | 15956473 | SAMSN1 | −1 |
| chr1 | 159796540 | 159795790 | 159797290 | SLAMF8 | 1 | chr21 | 15918681 | 15917931 | 15919431 | SAMSN1 | −1 |
| chr1 | 9129775 | 9129025 | 9130525 | SLC2A5 | −1 | chr21 | 43816955 | 43816205 | 43817705 | TMPRSS3 | −1 |
| chr1 | 205782304 | 205781554 | 205783054 | SLC41A1 | −1 | chr21 | 43809258 | 43808508 | 43810008 | TMPRSS3 | −1 |
| chr1 | 214454576 | 214453826 | 214455326 | SMYD2 | 1 | chr21 | 43816200 | 43815450 | 43816950 | TMPRSS3 | −1 |
| chr1 | 26232957 | 26232207 | 26233707 | STMN1 | −1 | chr22 | 39378414 | 39377664 | 39379164 | APOBEC3B | 1 |
| chr1 | 202612581 | 202611831 | 202613331 | SYT2 | −1 | chr22 | 39417118 | 39416368 | 39417868 | APOBEC3D | 1 |
| chr1 | 202679551 | 202678801 | 202680301 | SYT2 | −1 | chr22 | 36649124 | 36648374 | 36649874 | APOL1 | 1 |
| chr1 | 27671364 | 27670614 | 27672114 | SYTL1 | 1 | chr22 | 36556977 | 36556227 | 36557727 | APOL3 | −1 |
| chr1 | 159895522 | 159894772 | 159896272 | TAGLN2 | −1 | chr22 | 36562225 | 36561475 | 36562975 | APOL3 | −1 |
| chr1 | 6615241 | 6614491 | 6615991 | TAS1R1 | 1 | chr22 | 36600879 | 36600129 | 36601629 | APOL4 | −1 |
| chr1 | 1266694 | 1265944 | 1267444 | TAS1R3 | 1 | chr22 | 43253408 | 43252658 | 43254158 | ARFGAP3 | −1 |
| chr1 | 118148556 | 118147806 | 118149306 | TENT5C | 1 | chr22 | 43506754 | 43506004 | 43507504 | BIK | 1 |
| chr1 | 92351787 | 92351037 | 92352537 | TGFBR3 | −1 | chr22 | 51017096 | 51016346 | 51017846 | CPT1B | −1 |
| chr1 | 28199079 | 28198329 | 28199829 | THEMIS2 | 1 | chr22 | 51016506 | 51015756 | 51017256 | CPT1B | −1 |
| chr1 | 223310629 | 223309879 | 223311379 | TLR5 | −1 | chr22 | 51016894 | 51016144 | 51017644 | CPT1B | −1 |
| chr1 | 12227060 | 12226310 | 12227810 | TNFRSF1B | 1 | chr22 | 27017928 | 27017178 | 27018678 | CRYBA4 | 1 |
| chr1 | 6526235 | 6525485 | 6526985 | TNFRSF25 | −1 | chr22 | 50765489 | 50764739 | 50766239 | FAM116B | −1 |
| chr1 | 12123434 | 12122684 | 12124184 | TNFRSF8 | 1 | chr22 | 23412540 | 23411790 | 23413290 | GNAZ | 1 |
| chr1 | 12185957 | 12185207 | 12186707 | TNFRSF8 | 1 | chr22 | 46692638 | 46691888 | 46693388 | GTSE1 | 1 |
| chr1 | 8000926 | 8000176 | 8001676 | TNFRSF9 | −1 | chr22 | 50689814 | 50689064 | 50690564 | HDAC10 | −1 |
| chr1 | 3569084 | 3568334 | 3569834 | TP73 | 1 | chr22 | 23922495 | 23921745 | 23923245 | IGLL1 | −1 |
| chr1 | 33647267 | 33646517 | 33648017 | TRIM62 | −1 | chr22 | 25714223 | 25713473 | 25714973 | IGLL3P | 1 |
| chr1 | 145438469 | 145437719 | 145439219 | TXNIP | 1 | chr22 | 38864083 | 38863333 | 38864833 | KDELR3 | 1 |
| chr1 | 20512578 | 20511828 | 20513328 | UBXN10 | 1 | chr22 | 44894178 | 44893428 | 44894928 | LDOC1L | −1 |
| chr1 | 165796768 | 165796018 | 165797518 | UCK2 | 1 | chr22 | 38071615 | 38070865 | 38072365 | LGALS1 | 1 |
| chr1 | 46769303 | 46768553 | 46770053 | UQCRH | 1 | chr22 | 37976087 | 37975337 | 37976837 | LGALS2 | −1 |
| chr1 | 101185316 | 101184566 | 101186066 | VCAM1 | 1 | chr22 | 30642840 | 30642090 | 30643590 | LIF | −1 |
| chr1 | 27816669 | 27815919 | 27817419 | WASF2 | −1 | chr22 | 42828401 | 42827651 | 42829151 | NFAM1 | −1 |
| chr1 | 228135599 | 228134849 | 228136349 | WNT9A | −1 | chr22 | 44395091 | 44394341 | 44395841 | PARVB | 1 |
| chr1 | 53018654 | 53017904 | 53019404 | ZCCHC11 | −1 | chr22 | 44420156 | 44419406 | 44420906 | PARVB | 1 |
| chr1 | 45672228 | 45671478 | 45672978 | ZSWIM5 | −1 | chr22 | 44422183 | 44421433 | 44422933 | PARVB | 1 |
| chr10 | 90751147 | 90750397 | 90751897 | ACTA2 | −1 | chr22 | 44464996 | 44464246 | 44465746 | PARVB | 1 |
| chr10 | 90712580 | 90711830 | 90713330 | ACTA2 | −1 | chr22 | 31688514 | 31687764 | 31689264 | PIK31P1 | −1 |
| chr10 | 104262468 | 104261718 | 104263218 | ACTR1A | −1 | chr22 | 45072965 | 45072215 | 45073715 | PRR5 | 1 |
| chr10 | 112836790 | 112836040 | 112837540 | ADRA2A | 1 | chr22 | 37640310 | 37639560 | 37641060 | RAC2 | −1 |
| chr10 | 45869675 | 45868925 | 45870425 | ALOX5 | 1 | chr22 | 41347363 | 41346613 | 41348113 | RBX1 | 1 |
| chr10 | 62149488 | 62148738 | 62150238 | ANK3 | −1 | chr22 | 24129161 | 24128411 | 24129911 | SMARCB1 | 1 |
| chr10 | 90611575 | 90610825 | 90612325 | ANKRD22 | −1 | chr22 | 29949771 | 29949021 | 29950521 | THOC5 | −1 |
| chr10 | 5708541 | 5707791 | 5709291 | ASB13 | −1 | chr22 | 37499696 | 37498946 | 37500446 | TMPRSS6 | −1 |
| chr10 | 7830092 | 7829342 | 7830842 | ATP5F1C | 1 | chr22 | 42322822 | 42322072 | 42323572 | TNFRSF13C | −1 |
| chr10 | 116853384 | 116852634 | 116854134 | ATRNL1 | 1 | chr22 | 50682993 | 50682243 | 50683743 | TUBGCP6 | −1 |
| chr10 | 98031297 | 98030547 | 98032047 | BLNK | −1 | chr22 | 24891028 | 24890278 | 24891778 | UPB1 | 1 |
| chr10 | 102046469 | 102045719 | 102047219 | BLOC1S2 | −1 | chr22 | 30163358 | 30162608 | 30164108 | UQCR10 | 1 |
| chr10 | 102046116 | 102045366 | 102046866 | BLOC1S2 | −1 | chr22 | 22599087 | 22598337 | 22599837 | VPREB1 | 1 |
| chr10 | 102747783 | 102747033 | 102748533 | C10orf2 | 1 | chr22 | 24096655 | 24095905 | 24097405 | VPREB3 | −1 |
| chr10 | 105218645 | 105217895 | 105219395 | CALHM1 | −1 | chr22 | 29196585 | 29195835 | 29197335 | XBP1 | −1 |
| chr10 | 86088419 | 86087669 | 86089169 | CCSER2 | 1 | chr3 | 58223233 | 58222483 | 58223983 | ABHD6 | 1 |
| chr10 | 62538212 | 62537462 | 62538962 | CDK1 | 1 | chr3 | 132036251 | 132035501 | 132037001 | ACP3\|ACPP | 1 |
| chr10 | 62538088 | 62537338 | 62538838 | CDK1 | 1 | chr3 | 57583135 | 57582385 | 57583885 | ARF4 | −1 |
| chr10 | 95256389 | 95255639 | 95257139 | CEP55 | 1 | chr3 | 11314102 | 11313352 | 11314852 | ATG7 | 1 |
| chr10 | 95256368 | 95255618 | 95257118 | CEP55 | 1 | chr3 | 187463515 | 187462765 | 187464265 | BCL6 | −1 |
| chr10 | 75541821 | 75541071 | 75542571 | CHCHD1 | 1 | chr3 | 187452695 | 187451945 | 187453445 | BCL6 | −1 |
| chr10 | 73724123 | 73723373 | 73724873 | CHST3 | 1 | chr3 | 187454285 | 187453535 | 187455035 | BCL6 | −1 |
| chr10 | 101989376 | 101988626 | 101990126 | CHUK | −1 | chr3 | 5020801 | 5020051 | 5021551 | BHLHE40 | 1 |
| chr10 | 126716459 | 126715709 | 126717209 | CTBP2 | −1 | chr3 | 112218408 | 112217658 | 112219158 | BTLA | −1 |
| chr10 | 126849103 | 126848353 | 126849853 | CTBP2 | −1 | chr3 | 128997683 | 128996933 | 128998433 | C3orf37\|HMCE | 1 |
| chr10 | 126849624 | 126848874 | 126850374 | CTBP2 | −1 | chr3 | 128997816 | 128997066 | 128998566 | C3orf37 | 1 |
| chr10 | 44880497 | 44879747 | 44881247 | CXCL12 | −1 | chr3 | 62861054 | 62860304 | 62861804 | CADPS | −1 |
| chr10 | 74033678 | 74032928 | 74034428 | DDIT4 | 1 | chr3 | 191046874 | 191046124 | 191047624 | CCDC50 | 1 |
| chr10 | 70715884 | 70715134 | 70716634 | DDX21 | 1 | chr3 | 46249887 | 46249137 | 46250637 | CCR1 | −1 |
| chr10 | 70716195 | 70715445 | 70716945 | DDX21 | 1 | chr3 | 46395235 | 46394485 | 46395985 | CCR2 | 1 |
| chr10 | 127569884 | 127569134 | 127570634 | DHX32 | −1 | chr3 | 32993066 | 32992316 | 32993816 | CCR4 | 1 |
| chr10 | 74114907 | 74114157 | 74115657 | DNAJB12 | −1 | chr3 | 46411633 | 46410883 | 46412383 | CCR5 | 1 |
| chr10 | 98064153 | 98063403 | 98064903 | DNTT | 1 | chr3 | 45927996 | 45927246 | 45928746 | CCR9 | 1 |
| chr10 | 128593978 | 128593228 | 128594728 | DOCK1 | 1 | chr3 | 112052003 | 112051253 | 112052753 | CD200 | 1 |
| chr10 | 64576115 | 64575365 | 64576865 | EGR2 | −1 | chr3 | 107809861 | 107809111 | 107810611 | CD47 | −1 |
| chr10 | 97515409 | 97514659 | 97516159 | ENTPD1 | 1 | chr3 | 119278449 | 119277699 | 119279199 | CD80 | −1 |
| chr10 | 97471535 | 97470785 | 97472285 | ENTPD1 | 1 | chr3 | 121774221 | 121773471 | 121774971 | CD86 | 1 |
| chr10 | 97515672 | 97514922 | 97516422 | ENTPD1 | 1 | chr3 | 3221394 | 3220644 | 3222144 | CRBN | −1 |
| chr10 | 15413058 | 15412308 | 15413808 | FAM171A1 | −1 | chr3 | 39323226 | 39322476 | 39323976 | CX3CR1 | −1 |
| chr10 | 90750414 | 90749664 | 90751164 | FAS | 1 | chr3 | 39321527 | 39320777 | 39322277 | CX3CR1 | −1 |
| chr10 | 8096656 | 8095906 | 8097406 | GATA3 | 1 | chr3 | 39321930 | 39321180 | 39322680 | CX3CR1 | −1 |
| chr10 | 5855512 | 5854762 | 5856262 | GDI2 | −1 | chr3 | 39322764 | 39322014 | 39323514 | CX3CR1 | −1 |
| chr10 | 131934663 | 131933913 | 131935413 | GLRX3 | 1 | chr3 | 45986511 | 45985761 | 45987261 | CXCR6 | 1 |
| chr10 | 113943521 | 113942771 | 113944271 | GPAM | −1 | chr3 | 186080026 | 186079276 | 186080776 | DGKG | −1 |
| chr10 | 94447945 | 94447195 | 94448695 | HHEX | 1 | chr3 | 38080696 | 38079946 | 38081446 | DLEC1 | 1 |
| chr10 | 118502085 | 118501335 | 118502835 | HSPA12A | −1 | chr3 | 186285192 | 186284442 | 186285942 | DNAJB11 | 1 |
| chr10 | 6104288 | 6103538 | 6105038 | IL2RA | −1 | chr3 | 58200398 | 58199648 | 58201148 | DNASE1L3 | −1 |
| chr10 | 15762124 | 15761374 | 15762874 | ITGA8 | −1 | chr3 | 121554062 | 121553312 | 121554812 | EAF2 | 1 |
| chr10 | 33246822 | 33246072 | 33247572 | ITGB1 | −1 | chr3 | 5229358 | 5228608 | 5230108 | EDEM1 | 1 |
| chr10 | 33224486 | 33223736 | 33225236 | ITGB1 | −1 | chr3 | 184971886 | 184971136 | 184972636 | EHHADH | −1 |
| chr10 | 33247293 | 33246543 | 33248043 | ITGB1 | −1 | chr3 | 186501336 | 186500586 | 186502086 | EIF4A2 | 1 |
| chr10 | 7708934 | 7708184 | 7709684 | ITIH5 | −1 | chr3 | 27763989 | 27763239 | 27764739 | EOMES | −1 |
| chr10 | 72238563 | 72237813 | 72239313 | KIAA1274 | 1 | chr3 | 134514104 | 134513354 | 134514854 | EPHB1 | 1 |
| chr10 | 94353043 | 94352293 | 94353793 | KIF11 | 1 | chr3 | 13590631 | 13589881 | 13591381 | FBLN2 | 1 |
| chr10 | 3827467 | 3826717 | 3828217 | KLF6 | −1 | chr3 | 13610239 | 13609489 | 13610989 | FBLN2 | 1 |
| chr10 | 91011815 | 91011065 | 91012565 | LIPA | −1 | chr3 | 33318914 | 33318164 | 33319664 | FBXL2 | 1 |
| chr10 | 112630662 | 112629912 | 112631412 | LOC282997 | −1 | chr3 | 196295559 | 196294809 | 196296309 | FBXO45 | 1 |
| chr10 | 30722866 | 30722116 | 30723616 | MAP3K8 | 1 | chr3 | 14860469 | 14859719 | 14861219 | FGD5 | 1 |
| chr10 | 13203579 | 13202829 | 13204329 | MCM10 | 1 | chr3 | 71179988 | 71179238 | 71180738 | FOXP1 | −1 |
| chr10 | 131265448 | 131264698 | 131266198 | MGMT | 1 | chr3 | 111852152 | 111851402 | 111852902 | GCET2 | −1 |
| chr10 | 129924649 | 129923899 | 129925399 | MKI67 | −1 | chr3 | 111852101 | 111851351 | 111852851 | GCSAM | −1 |
| chr10 | 21823272 | 21822522 | 21824022 | MLLT10 | 1 | chr3 | 52719936 | 52719186 | 52720686 | GNL3 | 1 |
| chr10 | 95241951 | 95241201 | 95242701 | MYOF | −1 | chr3 | 169755717 | 169754967 | 169756467 | GPR160 | 1 |
| chr10 | 104154354 | 104153604 | 104155104 | NFKB2 | 1 | chr3 | 150920979 | 150920229 | 150921729 | GPR171 | −1 |
| chr10 | 103911933 | 103911183 | 103912683 | NOLC1 | 1 | chr3 | 119812513 | 119811763 | 119813263 | GSK3B | −1 |
| chr10 | 126107505 | 126106755 | 126108255 | OAT | −1 | chr3 | 112709765 | 112709015 | 112710515 | GTPBP8 | 1 |
| chr10 | 45811056 | 45810306 | 45811806 | OR13A1 | −1 | chr3 | 124774802 | 124774052 | 124775552 | HEG1 | −1 |
| chr10 | 89419476 | 89418726 | 89420226 | PAPSS2 | 1 | chr3 | 11294385 | 11293635 | 11295135 | HRH1 | 1 |
| chr10 | 97050781 | 97050031 | 97051531 | PDLIM1 | −1 | chr3 | 11178778 | 11178028 | 11179528 | HRH1 | 1 |
| chr10 | 98480271 | 98479521 | 98481021 | PIK3AP1 | −1 | chr3 | 11196213 | 11195463 | 11196963 | HRH1 | 1 |
| chr10 | 71993183 | 71992433 | 71993933 | PPA1 | −1 | chr3 | 11267668 | 11266918 | 11268418 | HRH1 | 1 |
| chr10 | 75255782 | 75255032 | 75256532 | PPP3CB | −1 | chr3 | 159706537 | 159705787 | 159707287 | IL12A | 1 |
| chr10 | 120938345 | 120937595 | 120939095 | PRDX3 | −1 | chr3 | 53880607 | 53879857 | 53881357 | IL17RB | 1 |
| chr10 | 72362531 | 72361781 | 72363281 | PRF1 | −1 | chr3 | 9944303 | 9943553 | 9945053 | IL17RE | 1 |
| chr10 | 89622870 | 89622120 | 89623620 | PTEN | 1 | chr3 | 9944511 | 9943761 | 9945261 | IL17RE | 1 |
| chr10 | 121302220 | 121301470 | 121302970 | RGS10 | −1 | chr3 | 121741051 | 121740301 | 121741801 | ILDR1 | −1 |
| chr10 | 121296045 | 121295295 | 121296795 | RGS10 | −1 | chr3 | 197686890 | 197686140 | 197687640 | IQCG | −1 |
| chr10 | 102279591 | 102278841 | 102280341 | SEC31B | −1 | chr3 | 197676796 | 197676046 | 197677546 | IQCG | −1 |
| chr10 | 13390297 | 13389547 | 13391047 | SEPHS1 | −1 | chr3 | 13009168 | 13008418 | 13009918 | IQSEC1 | −1 |
| chr10 | 70847874 | 70847124 | 70848624 | SRGN | 1 | chr3 | 128879891 | 128879141 | 128880641 | ISY1 | −1 |
| chr10 | 104404253 | 104403503 | 104405003 | TRIM8 | 1 | chr3 | 124606144 | 124605394 | 124606894 | ITGB5 | −1 |
| chr10 | 105155780 | 105155030 | 105156530 | USMG5 | −1 | chr3 | 19189946 | 19189196 | 19190696 | KCNH8 | 1 |
| chr10 | 11574274 | 11573524 | 11575024 | USP6NL | −1 | chr3 | 49170551 | 49169801 | 49171301 | LAMB2 | −1 |
| chr10 | 11653679 | 11652929 | 11654429 | USP6NL | −1 | chr3 | 182880699 | 182879949 | 182881449 | LAMP3 | −1 |
| chr10 | 75757872 | 75757122 | 75758622 | VCL | 1 | chr3 | 45636351 | 45635601 | 45637101 | LIMD1 | 1 |
| chr10 | 17270258 | 17269508 | 17271008 | VIM | 1 | chr3 | 120068186 | 120067436 | 120068936 | LRRC58 | −1 |
| chr10 | 28822348 | 28821598 | 28823098 | WAC | 1 | chr3 | 185080908 | 185080158 | 185081658 | MAP3K13 | 1 |
| chr10 | 77161664 | 77160914 | 77162414 | ZNF503 | −1 | chr3 | 129158852 | 129158102 | 129159602 | MBD4 | −1 |
| chr10 | 58121036 | 58120286 | 58121786 | ZWINT | −1 | chr3 | 127317066 | 127316316 | 127317816 | MCM2 | 1 |
| chr11 | 34379555 | 34378805 | 34380305 | ABTB2 | −1 | chr3 | 69788586 | 69787836 | 69789336 | MITF | 1 |
| chr11 | 442011 | 441261 | 442761 | ANO9 | −1 | chr3 | 69812706 | 69811956 | 69813456 | MITF | 1 |
| chr11 | 2292182 | 2291432 | 2292932 | ASCL2 | −1 | chr3 | 69812961 | 69812211 | 69813711 | MITF | 1 |
| chr11 | 108093559 | 108092809 | 108094309 | ATM | 1 | chr3 | 69915374 | 69914624 | 69916124 | MITF | 1 |
| chr11 | 118271869 | 118271119 | 118272619 | ATP5L | 1 | chr3 | 69985750 | 69985000 | 69986500 | MITF | 1 |
| chr11 | 134262247 | 134261497 | 134262997 | B3GAT1 | −1 | chr3 | 154797436 | 154796686 | 154798186 | MME | 1 |
| chr11 | 64052111 | 64051361 | 64052861 | BAD | −1 | chr3 | 154797704 | 154796954 | 154798454 | MME | 1 |
| chr11 | 102188226 | 102187476 | 102188976 | BIRC3 | 1 | chr3 | 154797952 | 154797202 | 154798702 | MME | 1 |
| chr11 | 33563876 | 33563126 | 33564626 | C11orf41 | 1 | chr3 | 154798078 | 154797328 | 154798828 | MME | 1 |
| chr11 | 122753473 | 122752723 | 122754223 | C11orf63 | 1 | chr3 | 49941070 | 49940320 | 49941820 | MST1R | −1 |
| chr11 | 34460472 | 34459722 | 34461222 | CAT | 1 | chr3 | 38179969 | 38179219 | 38180719 | MYD88 | 1 |
| chr11 | 64107695 | 64106945 | 64108445 | CCDC88B | 1 | chr3 | 179322490 | 179321740 | 179323240 | NDUFB5 | 1 |
| chr11 | 69455855 | 69455105 | 69456605 | CCND1 | 1 | chr3 | 101568358 | 101567608 | 101569108 | NFKBIZ | 1 |
| chr11 | 118213459 | 118212709 | 118214209 | CD3D | −1 | chr3 | 101546833 | 101546083 | 101547583 | NFKBIZ | 1 |
| chr11 | 118175260 | 118174510 | 118176010 | CD3E | 1 | chr3 | 196366557 | 196365807 | 196367307 | NRROS | 1 |
| chr11 | 118215071 | 118214321 | 118215821 | CD3G | 1 | chr3 | 9791628 | 9790878 | 9792378 | OGG1 | 1 |
| chr11 | 35160728 | 35159978 | 35161478 | CD44 | 1 | chr3 | 193310933 | 193310183 | 193311683 | OPA1 | 1 |
| chr11 | 60869867 | 60869117 | 60870617 | CD5 | 1 | chr3 | 151102600 | 151101850 | 151103350 | P2RY12 | −1 |
| chr11 | 33757985 | 33757235 | 33758735 | CD59 | −1 | chr3 | 122296449 | 122295699 | 122297199 | PARP15 | 1 |
| chr11 | 60739152 | 60738402 | 60739902 | CD6 | 1 | chr3 | 122334524 | 122333774 | 122335274 | PARP15 | 1 |
| chr11 | 2398524 | 2397774 | 2399274 | CD81 | 1 | chr3 | 122785917 | 122785167 | 122786667 | PDIA5 | 1 |
| chr11 | 64851623 | 64850873 | 64852373 | CDCA5 | −1 | chr3 | 73674091 | 73673341 | 73674841 | PDZRN3 | −1 |
| chr11 | 65626886 | 65626136 | 65627636 | CFL1 | −1 | chr3 | 178866311 | 178865561 | 178867061 | PIK3CA | 1 |
| chr11 | 46299212 | 46298462 | 46299962 | CREB3L1 | 1 | chr3 | 145878954 | 145878204 | 145879704 | PLOD2 | −1 |
| chr11 | 122709208 | 122708458 | 122709958 | CRTAM | 1 | chr3 | 146262651 | 146261901 | 146263401 | PLSCR1 | −1 |
| chr11 | 118754475 | 118753725 | 118755225 | CXCR5 | 1 | chr3 | 48470872 | 48470122 | 48471622 | PLXNB1 | −1 |
| chr11 | 118764100 | 118763350 | 118764850 | CXCR5 | 1 | chr3 | 48471460 | 48470710 | 48472210 | PLXNB1 | −1 |
| chr11 | 7695439 | 7694689 | 7696189 | CYB5R2 | −1 | chr3 | 121264853 | 121264103 | 121265603 | POLQ | −1 |
| chr11 | 6677085 | 6676335 | 6677835 | DCHS1 | −1 | chr3 | 119379437 | 119378687 | 119380187 | POPDC2 | −1 |
| chr11 | 102980304 | 102979554 | 102981054 | DYNC2H1 | 1 | chr3 | 64211131 | 64210381 | 64211881 | PRICKLE2 | −1 |
| chr11 | 61659006 | 61658256 | 61659756 | FADS3 | −1 | chr3 | 113775582 | 113774832 | 113776332 | QTRTD1 | 1 |
| chr11 | 94277017 | 94276267 | 94277767 | FUT4 | 1 | chr3 | 186524484 | 186523734 | 186525234 | RFC4 | −1 |
| chr11 | 118966177 | 118965427 | 118966927 | H2AX | −1 | chr3 | 186524290 | 186523540 | 186525040 | RFC4 | −1 |
| chr11 | 46638777 | 46638027 | 46639527 | HARBI1 | −1 | chr3 | 16555213 | 16554463 | 16555963 | RFTN1 | −1 |
| chr11 | 113845797 | 113845047 | 113846547 | HTR3A | 1 | chr3 | 49449635 | 49448885 | 49450385 | RHOA | −1 |
| chr11 | 113848221 | 113847471 | 113848971 | HTR3A | 1 | chr3 | 79816965 | 79816215 | 79817715 | ROBO1 | −1 |
| chr11 | 118927848 | 118927098 | 118928598 | HYOU1 | −1 | chr3 | 40498831 | 40498081 | 40499581 | RPL14 | 1 |
| chr11 | 308163 | 307413 | 308913 | IFITM2 | 1 | chr3 | 40498782 | 40498032 | 40499532 | RPL14 | 1 |
| chr11 | 117857063 | 117856313 | 117857813 | IL10RA | 1 | chr3 | 101405626 | 101404876 | 101406376 | RPL24 | −1 |
| chr11 | 71710109 | 71709359 | 71710859 | IL18BP | 1 | chr3 | 127842667 | 127841917 | 127843417 | RUVBL1 | −1 |
| chr11 | 71709957 | 71709207 | 71710707 | IL18BP | 1 | chr3 | 50305039 | 50304289 | 50305789 | SEMA3B | 1 |
| chr11 | 71710662 | 71709912 | 71711412 | IL18BP | 1 | chr3 | 15374136 | 15373386 | 15374886 | SH3BP5 | −1 |
| chr11 | 71710972 | 71710222 | 71711722 | IL18BP | 1 | chr3 | 15382901 | 15382151 | 15383651 | SH3BP5 | −1 |
| chr11 | 10715299 | 10714549 | 10716049 | IRAG1 | −1 | chr3 | 150481164 | 150480414 | 150481914 | SIAH2 | −1 |
| chr11 | 128712429 | 128711679 | 128713179 | KCNJ1 | −1 | chr3 | 141868363 | 141867613 | 141869113 | TFDP2 | −1 |
| chr11 | 111411384 | 111410634 | 111412134 | LAYN | 1 | chr3 | 195809060 | 195808310 | 195809810 | TFRC | −1 |
| chr11 | 18417813 | 18417063 | 18418563 | LDHA | 1 | chr3 | 30648093 | 30647343 | 30648843 | TGFBR2 | 1 |
| chr11 | 18415935 | 18415185 | 18416685 | LDHA | 1 | chr3 | 149095652 | 149094902 | 149096402 | TM4SF1 | −1 |
| chr11 | 33913836 | 33913086 | 33914586 | LMO2 | −1 | chr3 | 100211463 | 100210713 | 100212213 | TMEM45A | 1 |
| chr11 | 33891371 | 33890621 | 33892121 | LMO2 | −1 | chr3 | 133380737 | 133379987 | 133381487 | TOPBP1 | −1 |
| chr11 | 33891509 | 33890759 | 33892259 | LMO2 | −1 | chr3 | 189349216 | 189348466 | 189349966 | TP63 | 1 |
| chr11 | 65265232 | 65264482 | 65265982 | MALAT1 | 1 | chr3 | 189507448 | 189506698 | 189508198 | TP63 | 1 |
| chr11 | 102668891 | 102668141 | 102669641 | MMP1 | −1 | chr3 | 132378310 | 132377560 | 132379060 | UBA5 | 1 |
| chr11 | 60223320 | 60222570 | 60224070 | MS4A1 | 1 | chr3 | 179370543 | 179369793 | 179371293 | USP13 | 1 |
| chr11 | 60163961 | 60163211 | 60164711 | MS4A14 | 1 | chr3 | 46069234 | 46068484 | 46069984 | XCR1 | −1 |
| chr11 | 69061622 | 69060872 | 69062372 | MYEOV | 1 | chr3 | 42947658 | 42946908 | 42948408 | ZNF662 | 1 |
| chr11 | 89867697 | 89866947 | 89868447 | NAALAD2 | 1 | chr3 | 42947401 | 42946651 | 42948151 | ZNF662 | 1 |
| chr11 | 112832158 | 112831408 | 112832908 | NCAM1 | 1 | chr4 | 100242556 | 100241806 | 100243306 | ADH1B | −1 |
| chr11 | 65190268 | 65189518 | 65191018 | NEAT1 | 1 | chr4 | 7941653 | 7940903 | 7942403 | AFAP1 | −1 |
| chr11 | 74699179 | 74698429 | 74699929 | NEU3 | 1 | chr4 | 113970832 | 113970082 | 113971582 | ANK2 | 1 |
| chr11 | 62573774 | 62573024 | 62574524 | NXF1 | −1 | chr4 | 80993717 | 80992967 | 80994467 | ANTXR2 | −1 |
| chr11 | 12398732 | 12397982 | 12399482 | PARVA | 1 | chr4 | 86396267 | 86395517 | 86397017 | ARHGAP24 | 1 |
| chr11 | 104034835 | 104034085 | 104035585 | PDGFD | −1 | chr4 | 102711764 | 102711014 | 102712514 | BANK1 | 1 |
| chr11 | 2950685 | 2949935 | 2951435 | PHLDA2 | −1 | chr4 | 102734982 | 102734232 | 102735732 | BANK1 | 1 |
| chr11 | 63304293 | 63303543 | 63305043 | PLAAT4 | 1 | chr4 | 185570629 | 185569879 | 185571379 | CASP3 | −1 |
| chr11 | 111250417 | 111249667 | 111251167 | POU2AF1 | −1 | chr4 | 122745087 | 122744337 | 122745837 | CCNA2 | −1 |
| chr11 | 64014413 | 64013663 | 64015163 | PPP1R14B | −1 | chr4 | 15779901 | 15779151 | 15780651 | CD38 | 1 |
| chr11 | 57158130 | 57157380 | 57158880 | PRG2 | −1 | chr4 | 104119566 | 104118816 | 104120316 | CENPE | −1 |
| chr11 | 14665163 | 14664413 | 14665913 | PSMA1 | −1 | chr4 | 10686489 | 10685739 | 10687239 | CLNK | −1 |
| chr11 | 48002113 | 48001363 | 48002863 | PTPRJ | 1 | chr4 | 84206067 | 84205317 | 84206817 | COQ2 | −1 |
| chr11 | 67159423 | 67158673 | 67160173 | RAD9A | 1 | chr4 | 156875069 | 156874319 | 156875819 | CTSO | −1 |
| chr11 | 67160775 | 67160025 | 67161525 | RAD9A | 1 | chr4 | 74735121 | 74734371 | 74735871 | CXCL1 | 1 |
| chr11 | 36589575 | 36588825 | 36590325 | RAG1 | 1 | chr4 | 76944650 | 76943900 | 76945400 | CXCL10 | −1 |
| chr11 | 65430565 | 65429815 | 65431315 | RELA | −1 | chr4 | 78432907 | 78432157 | 78433657 | CXCL13 | 1 |
| chr11 | 114310108 | 114309358 | 114310858 | REXO2 | 1 | chr4 | 76928641 | 76927891 | 76929391 | CXCL9 | −1 |
| chr11 | 65488418 | 65487668 | 65489168 | RNASEH2C | −1 | chr4 | 100738003 | 100737253 | 100738753 | DAPP1 | 1 |
| chr11 | 117103452 | 117102702 | 117104202 | RNF214 | 1 | chr4 | 71859156 | 71858406 | 71859906 | DCK | 1 |
| chr11 | 117103403 | 117102653 | 117104153 | RNF214 | 1 | chr4 | 3465038 | 3464288 | 3465788 | DOK7 | 1 |
| chr11 | 8703958 | 8703208 | 8704708 | RPL27A | 1 | chr4 | 3486270 | 3485520 | 3487020 | DOK7 | 1 |
| chr11 | 14380730 | 14379980 | 14381480 | RRAS2 | −1 | chr4 | 17783135 | 17782385 | 17783885 | FAM184B | −1 |
| chr11 | 14380031 | 14379281 | 14380781 | RRAS2 | −1 | chr4 | 71091788 | 71091038 | 71092538 | FDCSP | 1 |
| chr11 | 14380073 | 14379323 | 14380823 | RRAS2 | −1 | chr4 | 144257915 | 144257165 | 144258665 | GAB1 | 1 |
| chr11 | 14386052 | 14385302 | 14386802 | RRAS2 | −1 | chr4 | 926086 | 925336 | 926836 | GAK | −1 |
| chr11 | 18287811 | 18287061 | 18288561 | SAA1 | 1 | chr4 | 22694547 | 22693797 | 22695297 | GBA3 | 1 |
| chr11 | 111957497 | 111956747 | 111958247 | SDHD | 1 | chr4 | 176923483 | 176922733 | 176924233 | GPM6A | −1 |
| chr11 | 57509044 | 57508294 | 57509794 | SELENOH | 1 | chr4 | 90229161 | 90228411 | 90229911 | GPRIN3 | −1 |
| chr11 | 57364991 | 57364241 | 57365741 | SERPING1 | 1 | chr4 | 108910870 | 108910120 | 108911620 | HADH | 1 |
| chr11 | 35440796 | 35440046 | 35441546 | SLC1A2 | −1 | chr4 | 89378268 | 89377518 | 89379018 | HERC5 | 1 |
| chr11 | 66139291 | 66138541 | 66140041 | SLC29A2 | −1 | chr4 | 57547872 | 57547122 | 57548622 | HOPX | −1 |
| chr11 | 811680 | 810930 | 812430 | SNORA52 | 1 | chr4 | 57522688 | 57521938 | 57523438 | HOPX | −1 |
| chr11 | 121322912 | 121322162 | 121323662 | SORL1 | 1 | chr4 | 95264027 | 95263277 | 95264777 | HPGDS | −1 |
| chr11 | 74660292 | 74659542 | 74661042 | SPCS2 | 1 | chr4 | 142557755 | 142557005 | 142558505 | IL15 | 1 |
| chr11 | 47399942 | 47399192 | 47400692 | SPI1 | −1 | chr4 | 71532377 | 71531627 | 71533127 | JCHAIN | −1 |
| chr11 | 9685624 | 9684874 | 9686374 | SWAP70 | 1 | chr4 | 55524085 | 55523335 | 55524835 | KIT | 1 |
| chr11 | 12766588 | 12765838 | 12767338 | TEAD1 | 1 | chr4 | 109089578 | 109088828 | 109090328 | LEF1 | −1 |
| chr11 | 119294838 | 119294088 | 119295588 | THY1 | −1 | chr4 | 4291896 | 4291146 | 4292646 | LYAR | −1 |
| chr11 | 111957522 | 111956772 | 111958272 | TIMM8B | −1 | chr4 | 4291748 | 4290998 | 4292498 | LYAR | −1 |
| chr11 | 6640692 | 6639942 | 6641442 | TPP1 | −1 | chr4 | 120988229 | 120987479 | 120988979 | MAD2L1 | −1 |
| chr11 | 73694352 | 73693602 | 73695102 | UCP2 | −1 | chr4 | 141075338 | 141074588 | 141076088 | MAML3 | −1 |
| chr11 | 119252436 | 119251686 | 119253186 | USP2 | −1 | chr4 | 17812525 | 17811775 | 17813275 | NCAPG | 1 |
| chr11 | 119234892 | 119234142 | 119235642 | USP2 | −1 | chr4 | 103422486 | 103421736 | 103423236 | NFKB1 | 1 |
| chr12 | 8765467 | 8764717 | 8766217 | AICDA | −1 | chr4 | 1894572 | 1893822 | 1895322 | NSD2 | 1 |
| chr12 | 112204726 | 112203976 | 112205476 | ALDH2 | 1 | chr4 | 57302304 | 57301554 | 57303054 | PAICS | 1 |
| chr12 | 47473648 | 47472898 | 47474398 | AMIGO2 | −1 | chr4 | 75858326 | 75857576 | 75859076 | PARM1 | 1 |
| chr12 | 110437328 | 110436578 | 110438078 | ANKRD13A | 1 | chr4 | 55095264 | 55094514 | 55096014 | PDGFRA | 1 |
| chr12 | 57873633 | 57872883 | 57874383 | ARHGAP9 | −1 | chr4 | 84035868 | 84035118 | 84036618 | PLAC8 | −1 |
| chr12 | 57871728 | 57870978 | 57872478 | ARHGAP9 | −1 | chr4 | 74486348 | 74485598 | 74487098 | RASSF6 | −1 |
| chr12 | 57873424 | 57872674 | 57874174 | ARHGAP9 | −1 | chr4 | 74486287 | 74485537 | 74487037 | RASSF6 | −1 |
| chr12 | 15114603 | 15113853 | 15115353 | ARHGDIB | −1 | chr4 | 37687998 | 37687248 | 37688748 | RELL1 | −1 |
| chr12 | 103351464 | 103350714 | 103352214 | ASCL1 | 1 | chr4 | 40198527 | 40197777 | 40199277 | RHOH | 1 |
| chr12 | 122457328 | 122456578 | 122458078 | BCL7A | 1 | chr4 | 39460508 | 39459758 | 39461258 | RPL9 | −1 |
| chr12 | 26278060 | 26277310 | 26278810 | BHLHE41 | −1 | chr4 | 152020725 | 152019975 | 152021475 | RPS3A | 1 |
| chr12 | 51717948 | 51717198 | 51718698 | BIN2 | −1 | chr4 | 77135046 | 77134296 | 77135796 | SCARB2 | −1 |
| chr12 | 107712190 | 107711440 | 107712940 | BTBD11 | 1 | chr4 | 8201094 | 8200344 | 8201844 | SH3TC1 | 1 |
| chr12 | 107974633 | 107973883 | 107975383 | BTBD11 | 1 | chr4 | 77356253 | 77355503 | 77357003 | SHROOM3 | 1 |
| chr12 | 92539673 | 92538923 | 92540423 | BTG1 | −1 | chr4 | 103266296 | 103265546 | 103267046 | SLC39A8 | −1 |
| chr12 | 103889749 | 103888999 | 103890499 | C12orf42 | −1 | chr4 | 139163503 | 139162753 | 139164253 | SLC7A11 | −1 |
| chr12 | 7261869 | 7261119 | 7262619 | C1RL | −1 | chr4 | 146403963 | 146403213 | 146404713 | SMAD1 | 1 |
| chr12 | 8219067 | 8218317 | 8219817 | C3AR1 | −1 | chr4 | 146402950 | 146402200 | 146403700 | SMAD1 | 1 |
| chr12 | 4382938 | 4382188 | 4383688 | CCND2 | 1 | chr4 | 39640710 | 39639960 | 39641460 | SMIM14 | −1 |
| chr12 | 69979114 | 69978364 | 69979864 | CCT2 | 1 | chr4 | 177241115 | 177240365 | 177241865 | SPCS3 | 1 |
| chr12 | 7656489 | 7655739 | 7657239 | CD163 | −1 | chr4 | 57687908 | 57687158 | 57688658 | SPINK2 | −1 |
| chr12 | 6554033 | 6553283 | 6554783 | CD27 | 1 | chr4 | 68424446 | 68423696 | 68425196 | STAP1 | 1 |
| chr12 | 6898638 | 6897888 | 6899388 | CD4 | 1 | chr4 | 106067450 | 106066700 | 106068200 | TET2 | 1 |
| chr12 | 9913497 | 9912747 | 9914247 | CD69 | −1 | chr4 | 154387501 | 154386751 | 154388251 | TMEM131L | 1 |
| chr12 | 6308881 | 6308131 | 6309631 | CD9 | 1 | chr4 | 146859623 | 146858873 | 146860373 | ZNF827 | −1 |
| chr12 | 6961230 | 6960480 | 6961980 | CDCA3 | −1 | chr5 | 157002783 | 157002033 | 157003533 | ADAM19 | −1 |
| chr12 | 12870058 | 12869308 | 12870808 | CDKN1B | 1 | chr5 | 148206156 | 148205406 | 148206906 | ADRB2 | 1 |
| chr12 | 122355768 | 122355018 | 122356518 | CFAP251\|WDR | 1 | chr5 | 139943845 | 139943095 | 139944595 | APBB3 | −1 |
| chr12 | 6716551 | 6715801 | 6717301 | CHD4 | −1 | chr5 | 149682516 | 149681766 | 149683266 | ARSI | −1 |
| chr12 | 104850751 | 104850001 | 104851501 | CHST11 | 1 | chr5 | 172410760 | 172410010 | 172411510 | ATP6V0E1 | 1 |
| chr12 | 10022735 | 10021985 | 10023485 | CLEC2B | −1 | chr5 | 17217669 | 17216919 | 17218419 | BASP1 | 1 |
| chr12 | 9822309 | 9821559 | 9823059 | CLEC2D | 1 | chr5 | 102594403 | 102593653 | 102595153 | C5orf30 | 1 |
| chr12 | 10183276 | 10182526 | 10184026 | CLEC9A | 1 | chr5 | 68462837 | 68462087 | 68463587 | CCNB1 | 1 |
| chr12 | 9885895 | 9885145 | 9886645 | CLECL1 | −1 | chr5 | 66492627 | 66491877 | 66493377 | CD180 | −1 |
| chr12 | 108733094 | 108732344 | 108733844 | CMKLR1 | −1 | chr5 | 149792314 | 149791564 | 149793064 | CD74 | −1 |
| chr12 | 108714439 | 108713689 | 108715189 | CMKLR1 | −1 | chr5 | 61808309 | 61807559 | 61809059 | CKS1B | −1 |
| chr12 | 12764826 | 12764076 | 12765576 | CREBL2 | 1 | chr5 | 157286151 | 157285401 | 157286901 | CLINT1 | −1 |
| chr12 | 91576900 | 91576150 | 91577650 | DCN | −1 | chr5 | 115420688 | 115419938 | 115421438 | COMMD10 | 1 |
| chr12 | 113495495 | 113494745 | 113496245 | DTX1 | 1 | chr5 | 139027877 | 139027127 | 139028627 | CXXC5 | 1 |
| chr12 | 12715317 | 12714567 | 12716067 | DUSP16 | −1 | chr5 | 176900199 | 176899449 | 176900949 | DBN1 | −1 |
| chr12 | 13349650 | 13348900 | 13350400 | EMP1 | 1 | chr5 | 79950802 | 79950052 | 79951552 | DHFR | −1 |
| chr12 | 53662083 | 53661333 | 53662833 | ESPL1 | 1 | chr5 | 176936858 | 176936108 | 176937608 | DOK3 | −1 |
| chr12 | 11802788 | 11802038 | 11803538 | ETV6 | 1 | chr5 | 176937427 | 176936677 | 176938177 | DOK3 | −1 |
| chr12 | 49319610 | 49318860 | 49320360 | FKBP11 | −1 | chr5 | 158526769 | 158526019 | 158527519 | EBF1 | −1 |
| chr12 | 50101197 | 50100447 | 50101947 | FMNL3 | −1 | chr5 | 137801179 | 137800429 | 137801929 | EGR1 | 1 |
| chr12 | 2986152 | 2985402 | 2986902 | FOXM1 | −1 | chr5 | 95297775 | 95297025 | 95298525 | ELL2 | −1 |
| chr12 | 6643093 | 6642343 | 6643843 | GAPDH | 1 | chr5 | 65222601 | 65221851 | 65223351 | ERBIN | 1 |
| chr12 | 110434029 | 110433279 | 110434779 | GIT2 | −1 | chr5 | 39270759 | 39270009 | 39271509 | FYB | −1 |
| chr12 | 75874460 | 75873710 | 75875210 | GLIPR1 | 1 | chr5 | 39219680 | 39218930 | 39220430 | FYB | −1 |
| chr12 | 96390143 | 96389393 | 96390893 | HAL | −1 | chr5 | 160975130 | 160974380 | 160975880 | GABRB2 | −1 |
| chr12 | 117319246 | 117318496 | 117319996 | HRK | −1 | chr5 | 95158418 | 95157668 | 95159168 | GLRX | −1 |
| chr12 | 104324112 | 104323362 | 104324862 | HSP90B1 | 1 | chr5 | 54398476 | 54397726 | 54399226 | GZMA | 1 |
| chr12 | 111126514 | 111125764 | 111127264 | HVCN1 | −1 | chr5 | 54320081 | 54319331 | 54320831 | GZMK | 1 |
| chr12 | 68553527 | 68552777 | 68554277 | IFNG | −1 | chr5 | 130501178 | 130500428 | 130501928 | HINT1 | −1 |
| chr12 | 68619601 | 68618851 | 68620351 | IL26 | −1 | chr5 | 132387654 | 132386904 | 132388404 | HSPA4 | 1 |
| chr12 | 113658899 | 113658149 | 113659649 | IQCD | −1 | chr5 | 137911133 | 137910383 | 137911883 | HSPA9 | −1 |
| chr12 | 25205446 | 25204696 | 25206196 | IRAG2 | 1 | chr5 | 140027355 | 140026605 | 140028105 | HK | 1 |
| chr12 | 108956377 | 108955627 | 108957127 | ISCU | 1 | chr5 | 131993865 | 131993115 | 131994615 | IL13 | 1 |
| chr12 | 54813244 | 54812494 | 54813994 | ITGA5 | −1 | chr5 | 55147334 | 55146584 | 55148084 | IL31RA | 1 |
| chr12 | 26986131 | 26985381 | 26986881 | ITPR2 | −1 | chr5 | 35856951 | 35856201 | 35857701 | IL7R | 1 |
| chr12 | 9760482 | 9759732 | 9761232 | KLRB1 | −1 | chr5 | 135231516 | 135230766 | 135232266 | IL9 | −1 |
| chr12 | 10460417 | 10459667 | 10461167 | KLRD1 | 1 | chr5 | 131826490 | 131825740 | 131827240 | IRF1 | −1 |
| chr12 | 10457049 | 10456299 | 10457799 | KLRD1 | 1 | chr5 | 52083730 | 52082980 | 52084480 | ITGA1 | 1 |
| chr12 | 10542617 | 10541867 | 10543367 | KLRK1 | −1 | chr5 | 156607837 | 156607087 | 156608587 | ITK | 1 |
| chr12 | 49449107 | 49448357 | 49449857 | KMT2D | −1 | chr5 | 169725231 | 169724481 | 169725981 | LCP2 | −1 |
| chr12 | 25403737 | 25402987 | 25404487 | KRA5 | −1 | chr5 | 169758434 | 169757684 | 169759184 | LOC257358 | 1 |
| chr12 | 118406788 | 118406038 | 118407538 | KSR2 | −1 | chr5 | 134735312 | 134734562 | 134736062 | MACROH2A1 | −1 |
| chr12 | 6881686 | 6880936 | 6882436 | LAG3 | 1 | chr5 | 56111401 | 56110651 | 56112151 | MAP3K1 | 1 |
| chr12 | 50677289 | 50676539 | 50678039 | LIMA1 | −1 | chr5 | 88199899 | 88199149 | 88200649 | MEF2C | −1 |
| chr12 | 54519854 | 54519104 | 54520604 | LOC400043 | 1 | chr5 | 1800009 | 1799259 | 1800759 | MRPL36 | −1 |
| chr12 | 108297548 | 108296798 | 108298298 | LOC728739 | −1 | chr5 | 68513587 | 68512837 | 68514337 | MRPS36 | 1 |
| chr12 | 91505608 | 91504858 | 91506358 | LUM | −1 | chr5 | 138725603 | 138724853 | 138726353 | MZB1 | −1 |
| chr12 | 69742121 | 69741371 | 69742871 | LYZ | 1 | chr5 | 140027370 | 140026620 | 140028120 | NDUFA2 | −1 |
| chr12 | 69201956 | 69201206 | 69202706 | MDM2 | 1 | chr5 | 177580968 | 177580218 | 177581718 | NHP2 | −1 |
| chr12 | 51317255 | 51316505 | 51318005 | METTL7A | 1 | chr5 | 170814652 | 170813902 | 170815402 | NPM1 | 1 |
| chr12 | 8815408 | 8814658 | 8816158 | MFAP5 | −1 | chr5 | 142783254 | 142782504 | 142784004 | NR3C1 | −1 |
| chr12 | 15038791 | 15038041 | 15039541 | MGP | −1 | chr5 | 142784045 | 142783295 | 142784795 | NR3C1 | −1 |
| chr12 | 81101277 | 81100527 | 81102027 | MYF6 | 1 | chr5 | 142815077 | 142814327 | 142815827 | NR3C1 | −1 |
| chr12 | 52416616 | 52415866 | 52417366 | NR4A1 | 1 | chr5 | 111312628 | 111311878 | 111313378 | NREP | −1 |
| chr12 | 52445185 | 52444435 | 52445935 | NR4A1 | 1 | chr5 | 111091948 | 111091198 | 111092698 | NREP | −1 |
| chr12 | 121477045 | 121476295 | 121477795 | OASL | −1 | chr5 | 111092945 | 111092195 | 111093695 | NREP | −1 |
| chr12 | 56498103 | 56497353 | 56498853 | PA2G4 | 1 | chr5 | 111093104 | 111092354 | 111093854 | NREP | −1 |
| chr12 | 80084877 | 80084127 | 80085627 | PAWR | −1 | chr5 | 111093106 | 111092356 | 111093856 | NREP | −1 |
| chr12 | 54943134 | 54942384 | 54943884 | PDE1B | 1 | chr5 | 111093252 | 111092502 | 111094002 | NREP | −1 |
| chr12 | 14721283 | 14720533 | 14722033 | PLBD1 | −1 | chr5 | 111093399 | 111092649 | 111094149 | NREP | −1 |
| chr12 | 102591623 | 102590873 | 102592373 | PMCH | −1 | chr5 | 111093793 | 111093043 | 111094543 | NREP | −1 |
| chr12 | 120105558 | 120104808 | 120106308 | PRKAB1 | 1 | chr5 | 111093915 | 111093165 | 111094665 | NREP | −1 |
| chr12 | 28124903 | 28124153 | 28125653 | PTHLH | −1 | chr5 | 38846101 | 38845351 | 38846851 | OSMR | 1 |
| chr12 | 7060434 | 7059684 | 7061184 | PTPN6 | 1 | chr5 | 38845959 | 38845209 | 38846709 | OSMR | 1 |
| chr12 | 7055739 | 7054989 | 7056489 | PTPN6 | 1 | chr5 | 131563508 | 131562758 | 131564258 | P4HA2 | −1 |
| chr12 | 71031220 | 71030470 | 71031970 | PTPRB | −1 | chr5 | 140739703 | 140738953 | 140740453 | PCDHGB2 | 1 |
| chr12 | 71003624 | 71002874 | 71004374 | PTPRB | −1 | chr5 | 131593364 | 131592614 | 131594114 | PDLIM4 | 1 |
| chr12 | 131356424 | 131355674 | 131357174 | RAN | 1 | chr5 | 52083774 | 52083024 | 52084524 | PELO | 1 |
| chr12 | 122232168 | 122231418 | 122232918 | RHOF | −1 | chr5 | 67511548 | 67510798 | 67512298 | PIK3R1 | 1 |
| chr12 | 112847400 | 112846650 | 112848150 | RPL6 | −1 | chr5 | 140373 | 139623 | 141123 | PLEKHG4B | 1 |
| chr12 | 64173583 | 64172833 | 64174333 | RXYLT1 | 1 | chr5 | 133561833 | 133561083 | 133562583 | PPP2CA | −1 |
| chr12 | 109025854 | 109025104 | 109026604 | SELPLG | −1 | chr5 | 159848917 | 159848167 | 159849667 | PTTG1 | 1 |
| chr12 | 109027670 | 109026920 | 109028420 | SELPLG | −1 | chr5 | 95066879 | 95066129 | 95067629 | RHOBTB3 | 1 |
| chr12 | 58013693 | 58012943 | 58014443 | SLC26A10 | 1 | chr5 | 179499118 | 179498368 | 179499868 | RNF130 | −1 |
| chr12 | 69139886 | 69139136 | 69140636 | SLC35E3 | 1 | chr5 | 81574396 | 81573646 | 81575146 | RPS23 | −1 |
| chr12 | 46662795 | 46662045 | 46663545 | SLC38A1 | −1 | chr5 | 133984479 | 133983729 | 133985229 | SEC24A | 1 |
| chr12 | 323689 | 322939 | 324439 | SLC6A12 | −1 | chr5 | 42812181 | 42811431 | 42812931 | SELENOP | −1 |
| chr12 | 24102637 | 24101887 | 24103387 | SOX5 | −1 | chr5 | 101632253 | 101631503 | 101633003 | SLCO4C1 | −1 |
| chr12 | 23737546 | 23736796 | 23738296 | SOX5 | −1 | chr5 | 135468536 | 135467786 | 135469286 | SMAD5 | 1 |
| chr12 | 24103966 | 24103216 | 24104716 | SOX5 | −1 | chr5 | 179247759 | 179247009 | 179248509 | SQSTM1 | 1 |
| chr12 | 24715383 | 24714633 | 24716133 | SOX5 | −1 | chr5 | 81047072 | 81046322 | 81047822 | SSBP2 | −1 |
| chr12 | 22487648 | 22486898 | 22488398 | ST8SIA1 | −1 | chr5 | 133450402 | 133449652 | 133451152 | TCF7 | 1 |
| chr12 | 57505216 | 57504466 | 57505966 | STAT6 | −1 | chr5 | 133451297 | 133450547 | 133452047 | TCF7 | 1 |
| chr12 | 16035329 | 16034579 | 16036079 | STRAP | 1 | chr5 | 133451349 | 133450599 | 133452099 | TCF7 | 1 |
| chr12 | 6451280 | 6450530 | 6452030 | TNFRSF1A | −1 | chr5 | 166711804 | 166711054 | 166712554 | TENM2 | 1 |
| chr12 | 3186543 | 3185793 | 3187293 | TSPAN9 | 1 | chr5 | 1295162 | 1294412 | 1295912 | TERT | −1 |
| chr12 | 49583107 | 49582357 | 49583857 | TUBA1A | −1 | chr5 | 892758 | 892008 | 893508 | TRIP13 | 1 |
| chr12 | 125399894 | 125399144 | 125400644 | UBC | −1 | chr5 | 132202252 | 132201502 | 132203002 | UQCRQ | 1 |
| chr12 | 95945266 | 95944516 | 95946016 | USP44 | −1 | chr5 | 82767284 | 82766534 | 82768034 | VCAN | 1 |
| chr12 | 95942620 | 95941870 | 95943370 | USP44 | −1 | chr5 | 133340824 | 133340074 | 133341574 | VDAC1 | −1 |
| chr12 | 862089 | 861339 | 862839 | WNK1 | 1 | chr5 | 133340657 | 133339907 | 133341407 | VDAC1 | −1 |
| chr12 | 64798130 | 64797380 | 64798880 | XPOT | 1 | chr5 | 140044261 | 140043511 | 140045011 | WDR55 | 1 |
| chr12 | 10875911 | 10875161 | 10876661 | YBX3 | −1 | chr5 | 43121700 | 43120950 | 43122450 | ZNF131 | 1 |
| chr12 | 69753483 | 69752733 | 69754233 | YEATS4 | 1 | chr6 | 139349819 | 139349069 | 139350569 | ABRACL | 1 |
| chr13 | 31309645 | 31308895 | 31310395 | ALOX5AP | 1 | chr6 | 11779036 | 11778286 | 11779786 | ADTRP | −1 |
| chr13 | 31287614 | 31286864 | 31288364 | ALOX5AP | 1 | chr6 | 32152101 | 32151351 | 32152851 | AGER | −1 |
| chr13 | 53029602 | 53028852 | 53030352 | CKAP2 | 1 | chr6 | 151561134 | 151560384 | 151561884 | AKAP12 | 1 |
| chr13 | 49280951 | 49280201 | 49281701 | CYSLTR2 | 1 | chr6 | 151646665 | 151645915 | 151647415 | AKAP12 | 1 |
| chr13 | 50656307 | 50655557 | 50657057 | DLEU1 | 1 | chr6 | 16761687 | 16760937 | 16762437 | ATXN1 | −1 |
| chr13 | 50699677 | 50698927 | 50700427 | DLEU2 | −1 | chr6 | 91006461 | 91005711 | 91007211 | BACH2 | −1 |
| chr13 | 107187462 | 107186712 | 107188212 | EFNB2 | −1 | chr6 | 26458153 | 26457403 | 26458903 | BTN2A1 | 1 |
| chr13 | 98795505 | 98794755 | 98796255 | FARP1 | 1 | chr6 | 26458177 | 26457427 | 26458927 | BTN2A1 | 1 |
| chr13 | 41240734 | 41239984 | 41241484 | FOXO1 | −1 | chr6 | 41888926 | 41888176 | 41889676 | BYSL | 1 |
| chr13 | 93879095 | 93878345 | 93879845 | GPC6 | 1 | chr6 | 31895254 | 31894504 | 31896004 | C2 | 1 |
| chr13 | 99913998 | 99913248 | 99914748 | GPR18 | −1 | chr6 | 31949801 | 31949051 | 31950551 | C4A | 1 |
| chr13 | 99959659 | 99958909 | 99960409 | GPR183 | −1 | chr6 | 25279656 | 25278906 | 25280406 | CARMIL1 | 1 |
| chr13 | 114018441 | 114017691 | 114019191 | GRTP1 | −1 | chr6 | 132272513 | 132271763 | 132273263 | CCN2 | −1 |
| chr13 | 27998681 | 27997931 | 27999431 | GTF3A | 1 | chr6 | 14117872 | 14117122 | 14118622 | CD83 | 1 |
| chr13 | 48807294 | 48806544 | 48808044 | ITM2B | 1 | chr6 | 14117486 | 14116736 | 14118236 | CD83 | 1 |
| chr13 | 40177308 | 40176558 | 40178058 | LHFPL6 | −1 | chr6 | 36646487 | 36645737 | 36647237 | CDKN1A | 1 |
| chr13 | 41345309 | 41344559 | 41346059 | MRPS31 | −1 | chr6 | 36644236 | 36643486 | 36644986 | CDKN1A | 1 |
| chr13 | 33112936 | 33112186 | 33113686 | N4BP2L2 | −1 | chr6 | 36644266 | 36643516 | 36645016 | CDKN1A | 1 |
| chr13 | 35516424 | 35515674 | 35517174 | NBEA | 1 | chr6 | 36646455 | 36645705 | 36647205 | CDKN1A | 1 |
| chr13 | 36050885 | 36050135 | 36051635 | NBEA | 1 | chr6 | 126661320 | 126660570 | 126662070 | CENPW | 1 |
| chr13 | 67804468 | 67803718 | 67805218 | PCDH9 | −1 | chr6 | 88860882 | 88860132 | 88861632 | CNR1 | −1 |
| chr13 | 27844464 | 27843714 | 27845214 | RASL11A | 1 | chr6 | 75960039 | 75959289 | 75960789 | COX7A2 | −1 |
| chr13 | 48877911 | 48877161 | 48878661 | RB1 | 1 | chr6 | 38690898 | 38690148 | 38691648 | DNAH8 | 1 |
| chr13 | 34392186 | 34391436 | 34392936 | RFC3 | 1 | chr6 | 292097 | 291347 | 292847 | DUSP22 | 1 |
| chr13 | 42031695 | 42030945 | 42032445 | RGCC | 1 | chr6 | 131949601 | 131948851 | 131950351 | ENPP3 | 1 |
| chr13 | 51483814 | 51483064 | 51484564 | RNASEH2B | 1 | chr6 | 6321067 | 6320317 | 6321817 | F13A1 | −1 |
| chr13 | 46961635 | 46960885 | 46962385 | RUBCNL | −1 | chr6 | 82462425 | 82461675 | 82463175 | FAM46A\|TENT | −1 |
| chr13 | 76056250 | 76055500 | 76057000 | TBC1D4 | −1 | chr6 | 112194655 | 112193905 | 112195405 | FYN | −1 |
| chr13 | 108922066 | 108921316 | 108922816 | TNFSF13B | 1 | chr6 | 112041265 | 112040515 | 112042015 | FYN | −1 |
| chr14 | 69445956 | 69445206 | 69446706 | ACTN1 | −1 | chr6 | 112080334 | 112079584 | 112081084 | FYN | −1 |
| chr14 | 24037279 | 24036529 | 24038029 | AP1G2 | −1 | chr6 | 2245926 | 2245176 | 2246676 | GMDS | −1 |
| chr14 | 20923354 | 20922604 | 20924104 | APEX1 | 1 | chr6 | 24775166 | 24774416 | 24775916 | GMNN | 1 |
| chr14 | 51026743 | 51025993 | 51027493 | ATL1 | 1 | chr6 | 24775640 | 24774890 | 24776390 | GMNN | 1 |
| chr14 | 50999799 | 50999049 | 51000549 | ATL1 | 1 | chr6 | 26056699 | 26055949 | 26057449 | H1-2 | −1 |
| chr14 | 75988768 | 75988018 | 75989518 | BATF | 1 | chr6 | 26124431 | 26123681 | 26125181 | H2AC6 | 1 |
| chr14 | 104387838 | 104387088 | 104388588 | C14orf2 | −1 | chr6 | 26104104 | 26103354 | 26104854 | H4C3 | 1 |
| chr14 | 98444461 | 98443711 | 98445211 | C14orf64 | −1 | chr6 | 26217165 | 26216415 | 26217915 | HIST1H2AE | 1 |
| chr14 | 90863327 | 90862577 | 90864077 | CALM1 | 1 | chr6 | 26216872 | 26216122 | 26217622 | HIST1H2BG | −1 |
| chr14 | 54863673 | 54862923 | 54864423 | CDKN3 | 1 | chr6 | 27100529 | 27099779 | 27101279 | HIST1H2BJ | −1 |
| chr14 | 54908148 | 54907398 | 54908898 | CNIH | −1 | chr6 | 27806440 | 27805690 | 27807190 | HIST1H2BN | 1 |
| chr14 | 105952654 | 105951904 | 105953404 | CRIP1 | 1 | chr6 | 27777842 | 27777092 | 27778592 | HIST1H3H | 1 |
| chr14 | 59730173 | 59729423 | 59730923 | DAAM1 | 1 | chr6 | 26285762 | 26285012 | 26286512 | HIST1H4H | −1 |
| chr14 | 24105582 | 24104832 | 24106332 | DHRS2 | 1 | chr6 | 12012571 | 12011821 | 12013321 | HIVEP1 | 1 |
| chr14 | 55658396 | 55657646 | 55659146 | DLGAP5 | −1 | chr6 | 31239863 | 31239113 | 31240613 | HLA-C | −1 |
| chr14 | 106384297 | 106383547 | 106385047 | FAM30A | 1 | chr6 | 32920899 | 32920149 | 32921649 | HLA-DMA | −1 |
| chr14 | 75745477 | 75744727 | 75746227 | FOS | 1 | chr6 | 32784825 | 32784075 | 32785575 | HLA-DOB | −1 |
| chr14 | 65877310 | 65876560 | 65878060 | FUT8 | 1 | chr6 | 33048487 | 33047737 | 33049237 | HLA-DPA1 | −1 |
| chr14 | 65879447 | 65878697 | 65880197 | FUT8 | 1 | chr6 | 32605134 | 32604384 | 32605884 | HLA-DQA1 | 1 |
| chr14 | 96000930 | 96000180 | 96001680 | GLRX5 | 1 | chr6 | 32407619 | 32406869 | 32408369 | HLA-DRA | 1 |
| chr14 | 25103473 | 25102723 | 25104223 | GZMB | −1 | chr6 | 32557625 | 32556875 | 32558375 | HLA-DRB1 | −1 |
| chr14 | 25078864 | 25078114 | 25079614 | GZMH | −1 | chr6 | 30457244 | 30456494 | 30457994 | HLA-E | 1 |
| chr14 | 24658170 | 24657420 | 24658920 | IPO4 | −1 | chr6 | 29691185 | 29690435 | 29691935 | HLA-F | 1 |
| chr14 | 75898837 | 75898087 | 75899587 | JDP2 | 1 | chr6 | 29794756 | 29794006 | 29795506 | HLA-G | 1 |
| chr14 | 75894508 | 75893758 | 75895258 | JDP2 | 1 | chr6 | 44215603 | 44214853 | 44216353 | HSP90AB1 | 1 |
| chr14 | 75894919 | 75894169 | 75895669 | JDP2 | 1 | chr6 | 31783291 | 31782541 | 31784041 | HSPA1A | 1 |
| chr14 | 75895059 | 75894309 | 75895809 | JDP2 | 1 | chr6 | 154651215 | 154650465 | 154651965 | IPCEF1 | −1 |
| chr14 | 93215024 | 93214274 | 93215774 | LGMN | −1 | chr6 | 154677900 | 154677150 | 154678650 | IPCEF1 | −1 |
| chr14 | 101292444 | 101291694 | 101293194 | MEG3 | 1 | chr6 | 391739 | 390989 | 392489 | IRF4 | 1 |
| chr14 | 101292468 | 101291718 | 101293218 | MEG3 | 1 | chr6 | 33588142 | 33587392 | 33588892 | ITPR3 | 1 |
| chr14 | 35873952 | 35873202 | 35874702 | NFKBIA | −1 | chr6 | 105404923 | 105404173 | 105405673 | LIN28B | 1 |
| chr14 | 75422487 | 75421737 | 75423237 | PGF | −1 | chr6 | 27729522 | 27728772 | 27730272 | LOC100131289 | 1 |
| chr14 | 20937538 | 20936788 | 20938288 | PNP | 1 | chr6 | 31554977 | 31554227 | 31555727 | LST1 | 1 |
| chr14 | 50155021 | 50154271 | 50155771 | POLE2 | −1 | chr6 | 31553955 | 31553205 | 31554705 | LST1 | 1 |
| chr14 | 61788435 | 61787685 | 61789185 | PRKCH | 1 | chr6 | 31554475 | 31553725 | 31555225 | LST1 | 1 |
| chr14 | 35761563 | 35760813 | 35762313 | PSMA6 | 1 | chr6 | 31539831 | 31539081 | 31540581 | LTA | 1 |
| chr14 | 52781113 | 52780363 | 52781863 | PTGER2 | 1 | chr6 | 31550202 | 31549452 | 31550952 | LTB | −1 |
| chr14 | 89021077 | 89020327 | 89021827 | PTPN21 | −1 | chr6 | 31637944 | 31637194 | 31638694 | LY6G5B | 1 |
| chr14 | 51411454 | 51410704 | 51412204 | PYGL | −1 | chr6 | 137113656 | 137112906 | 137114406 | MAP3K5 | −1 |
| chr14 | 21249210 | 21248460 | 21249960 | RNASE6 | 1 | chr6 | 91296764 | 91296014 | 91297514 | MAP3K7 | −1 |
| chr14 | 75390005 | 75389255 | 75390755 | RPS6KL1 | −1 | chr6 | 136847610 | 136846860 | 136848360 | MAP7 | −1 |
| chr14 | 94857029 | 94856279 | 94857779 | SERPINA1 | −1 | chr6 | 114178541 | 114177791 | 114179291 | MARCKS | 1 |
| chr14 | 94855153 | 94854403 | 94855903 | SERPINA1 | −1 | chr6 | 20212630 | 20211880 | 20213380 | MBOAT1 | −1 |
| chr14 | 94942584 | 94941834 | 94943334 | SERPINA9 | −1 | chr6 | 84140790 | 84140040 | 84141540 | ME1 | −1 |
| chr14 | 34931562 | 34930812 | 34932312 | SPTSSA | −1 | chr6 | 132722684 | 132721934 | 132723434 | MOXD1 | −1 |
| chr14 | 25519503 | 25518753 | 25520253 | STXBP6 | −1 | chr6 | 44095194 | 44094444 | 44095944 | MRPL14 | −1 |
| chr14 | 96180533 | 96179783 | 96181283 | TCL1A | −1 | chr6 | 76459049 | 76458299 | 76459799 | MYO6 | 1 |
| chr14 | 96117514 | 96116764 | 96118264 | TCL6 | 1 | chr6 | 138428648 | 138427898 | 138429398 | PERP | −1 |
| chr14 | 71108504 | 71107754 | 71109254 | TTC9 | 1 | chr6 | 12718542 | 12717792 | 12719292 | PHACTR1 | 1 |
| chr14 | 100843142 | 100842392 | 100843892 | WARS | −1 | chr6 | 37137979 | 37137229 | 37138729 | PIM1 | 1 |
| chr14 | 55493795 | 55493045 | 55494545 | WDHD1 | −1 | chr6 | 106534195 | 106533445 | 106534945 | PRDM1 | 1 |
| chr15 | 74833518 | 74832768 | 74834268 | ARID3B | 1 | chr6 | 106546736 | 106545986 | 106547486 | PRDM1 | 1 |
| chr15 | 45003675 | 45002925 | 45004425 | B2M | 1 | chr6 | 43044552 | 43043802 | 43045302 | PTK7 | 1 |
| chr15 | 80263788 | 80263038 | 80264538 | BCL2A1 | −1 | chr6 | 43044005 | 43043255 | 43044755 | PTK7 | 1 |
| chr15 | 52404972 | 52404222 | 52405722 | BCL2L10 | −1 | chr6 | 3752260 | 3751510 | 3753010 | PXDC1 | −1 |
| chr15 | 91260558 | 91259808 | 91261308 | BLM | 1 | chr6 | 107077453 | 107076703 | 107078203 | QRSL1 | 1 |
| chr15 | 40401075 | 40400325 | 40401825 | BMF | −1 | chr6 | 13711796 | 13711046 | 13712546 | RANBP9 | −1 |
| chr15 | 40398287 | 40397537 | 40399037 | BMF | −1 | chr6 | 24911195 | 24910445 | 24911945 | RIPOR2 | −1 |
| chr15 | 40398639 | 40397889 | 40399389 | BMF | −1 | chr6 | 24877583 | 24876833 | 24878333 | RIPOR2 | −1 |
| chr15 | 40453227 | 40452477 | 40453977 | BUB1B | 1 | chr6 | 32146231 | 32145481 | 32146981 | RNF5P1 | 1 |
| chr15 | 45722805 | 45722055 | 45723555 | C15orf48 | 1 | chr6 | 127439749 | 127438999 | 127440499 | RSPO3 | 1 |
| chr15 | 59397277 | 59396527 | 59398027 | CCNB2 | 1 | chr6 | 2903514 | 2902764 | 2904264 | SERPINB9 | −1 |
| chr15 | 75074398 | 75073648 | 75075148 | CSK | 1 | chr6 | 134639196 | 134638446 | 134639946 | SGK1 | −1 |
| chr15 | 79237433 | 79236683 | 79238183 | CTSH | −1 | chr6 | 134496034 | 134495284 | 134496784 | SGK1 | −1 |
| chr15 | 44069741 | 44068991 | 44070491 | ELL3 | −1 | chr6 | 134497070 | 134496320 | 134497820 | SGK1 | −1 |
| chr15 | 34394149 | 34393399 | 34394899 | EMC7 | −1 | chr6 | 134499010 | 134498260 | 134499760 | SGK1 | −1 |
| chr15 | 41056218 | 41055468 | 41056968 | GCHFR | 1 | chr6 | 111408781 | 111408031 | 111409531 | SLC16A10 | 1 |
| chr15 | 83621480 | 83620730 | 83622230 | HOMER2 | −1 | chr6 | 46620678 | 46619928 | 46621428 | SLC25A27 | 1 |
| chr15 | 90645736 | 90644986 | 90646486 | IDH2 | −1 | chr6 | 158244296 | 158243546 | 158245046 | SNX9 | 1 |
| chr15 | 81517741 | 81516991 | 81518491 | IL16 | 1 | chr6 | 144471663 | 144470913 | 144472413 | STX11 | 1 |
| chr15 | 89181974 | 89181224 | 89182724 | ISG20 | 1 | chr6 | 158402919 | 158402169 | 158403669 | SYNJ2 | 1 |
| chr15 | 69706688 | 69705938 | 69707438 | KIF23 | 1 | chr6 | 159082351 | 159081601 | 159083101 | SYTL3 | 1 |
| chr15 | 71146498 | 71145748 | 71147248 | LARP6 | −1 | chr6 | 159466184 | 159465434 | 159466934 | TAGAP | −1 |
| chr15 | 31523050 | 31522300 | 31523800 | LOC283710 | −1 | chr6 | 155153831 | 155153081 | 155154581 | TIAM2 | 1 |
| chr15 | 100105905 | 100105155 | 100106655 | MEF2A | 1 | chr6 | 10747992 | 10747242 | 10748742 | TMEM14B | 1 |
| chr15 | 59665099 | 59664349 | 59665849 | MYO1E | −1 | chr6 | 52441713 | 52440963 | 52442463 | TRAM2 | −1 |
| chr15 | 23932450 | 23931700 | 23933200 | NDN | −1 | chr6 | 41254457 | 41253707 | 41255207 | TREM1 | −1 |
| chr15 | 41625069 | 41624319 | 41625819 | NUSAP1 | 1 | chr6 | 80714322 | 80713572 | 80715072 | TTK | 1 |
| chr15 | 65134088 | 65133338 | 65134838 | PLEKHO2 | 1 | chr6 | 30687978 | 30687228 | 30688728 | TUBB | 1 |
| chr15 | 55562592 | 55561842 | 55563342 | RAB27A | −1 | chr6 | 3157760 | 3157010 | 3158510 | TUBB2A | −1 |
| chr15 | 55562484 | 55561734 | 55563234 | RAB27A | −1 | chr6 | 3227969 | 3227219 | 3228719 | TUBB2B | −1 |
| chr15 | 55563107 | 55562357 | 55563857 | RAB27A | −1 | chr6 | 7911047 | 7910297 | 7911797 | TXNDC5 | −1 |
| chr15 | 55582013 | 55581263 | 55582763 | RAB27A | −1 | chr6 | 7910310 | 7909560 | 7911060 | TXNDC5 | −1 |
| chr15 | 79383115 | 79382365 | 79383865 | RASGRF1 | −1 | chr6 | 90062567 | 90061817 | 90063317 | UBE2J1 | −1 |
| chr15 | 77223960 | 77223210 | 77224710 | RCN2 | 1 | chr6 | 149068464 | 149067714 | 149069214 | UST | 1 |
| chr15 | 66797221 | 66796471 | 66797971 | RPL4 | −1 | chr6 | 43738444 | 43737694 | 43739194 | VEGFA | 1 |
| chr15 | 63450001 | 63449251 | 63450751 | RPS27L | −1 | chr6 | 133079147 | 133078397 | 133079897 | VNN2 | −1 |
| chr15 | 55489265 | 55488515 | 55490015 | RSL24D1 | −1 | chr6 | 110501207 | 110500457 | 110501957 | WASF1 | −1 |
| chr15 | 33603177 | 33602427 | 33603927 | RYR3 | 1 | chr6 | 109804440 | 109803690 | 109805190 | ZBTB24 | −1 |
| chr15 | 90744551 | 90743801 | 90745301 | SEMA4B | 1 | chr7 | 121773876 | 121773126 | 121774626 | AASS | −1 |
| chr15 | 90728151 | 90727401 | 90728901 | SEMA4B | 1 | chr7 | 87342564 | 87341814 | 87343314 | ABCB1 | −1 |
| chr15 | 74726808 | 74726058 | 74727558 | SEMA7A | −1 | chr7 | 5570340 | 5569590 | 5571090 | ACTB | −1 |
| chr15 | 50474393 | 50473643 | 50475143 | SLC27A2 | 1 | chr7 | 45614121 | 45613371 | 45614871 | ADCY1 | 1 |
| chr15 | 78369994 | 78369244 | 78370744 | TBC1D2B | −1 | chr7 | 17338246 | 17337496 | 17338996 | AHR | 1 |
| chr15 | 30114706 | 30113956 | 30115456 | TJP1 | −1 | chr7 | 6048876 | 6048126 | 6049626 | AIMP2 | 1 |
| chr15 | 85144236 | 85143486 | 85144986 | ZSCAN2 | 1 | chr7 | 91570181 | 91569431 | 91570931 | AKAP9 | 1 |
| chr16 | 30064444 | 30063694 | 30065194 | ALDOA | 1 | chr7 | 36429415 | 36428665 | 36430165 | ANLN | 1 |
| chr16 | 25026652 | 25025902 | 25027402 | ARHGAP17 | −1 | chr7 | 69064319 | 69063569 | 69065069 | AUTS2 | 1 |
| chr16 | 57279010 | 57278260 | 57279760 | ARL2BP | 1 | chr7 | 97840739 | 97839989 | 97841489 | BHLHA15 | 1 |
| chr16 | 67515140 | 67514390 | 67515890 | ATP6V0D1 | −1 | chr7 | 43798286 | 43797536 | 43799036 | BLVRA | 1 |
| chr16 | 15528152 | 15527402 | 15528902 | C16orf45 | 1 | chr7 | 140624564 | 140623814 | 140625314 | BRAF | −1 |
| chr16 | 57438679 | 57437929 | 57439429 | CCL17 | 1 | chr7 | 40174574 | 40173824 | 40175324 | C7orf10 | 1 |
| chr16 | 57392684 | 57391934 | 57393434 | CCL22 | 1 | chr7 | 3083579 | 3082829 | 3084329 | CARD11 | −1 |
| chr16 | 28943260 | 28942510 | 28944010 | CD19 | 1 | chr7 | 116164839 | 116164089 | 116165589 | CAV1 | 1 |
| chr16 | 68771128 | 68770378 | 68771878 | CDH1 | 1 | chr7 | 116165062 | 116164312 | 116165812 | CAV1 | 1 |
| chr16 | 2581420 | 2580670 | 2582170 | CEMP1 | −1 | chr7 | 116166346 | 116165596 | 116167096 | CAV1 | 1 |
| chr16 | 10971055 | 10970305 | 10971805 | CIJTA | 1 | chr7 | 116166411 | 116165661 | 116167161 | CAV1 | 1 |
| chr16 | 66600350 | 66599600 | 66601100 | CMTM1 | 1 | chr7 | 26240782 | 26240032 | 26241532 | CBX3 | 1 |
| chr16 | 66600293 | 66599543 | 66601043 | CMTM1 | 1 | chr7 | 75419214 | 75418464 | 75419964 | CCL26 | −1 |
| chr16 | 88717560 | 88716810 | 88718310 | CYBA | −1 | chr7 | 21985702 | 21984952 | 21986452 | CDCA7L | −1 |
| chr16 | 50776671 | 50775921 | 50777421 | CYLD | 1 | chr7 | 90338712 | 90337962 | 90339462 | CDK14 | 1 |
| chr16 | 30125177 | 30124427 | 30125927 | GDPD3 | −1 | chr7 | 73703805 | 73703055 | 73704555 | CLIP2 | 1 |
| chr16 | 23521815 | 23521065 | 23522565 | GGA2 | −1 | chr7 | 94023873 | 94023123 | 94024623 | COL1A2 | 1 |
| chr16 | 4364762 | 4364012 | 4365512 | GLIS2 | 1 | chr7 | 137686803 | 137686053 | 137687553 | CREB3L2 | −1 |
| chr16 | 19896239 | 19895489 | 19896989 | GPRC5B | −1 | chr7 | 101459291 | 101458541 | 101460041 | CUX1 | 1 |
| chr16 | 12009777 | 12009027 | 12010527 | GSPT1 | −1 | chr7 | 101459183 | 101458433 | 101459933 | CUX1 | 1 |
| chr16 | 83841448 | 83840698 | 83842198 | HSBP1 | 1 | chr7 | 101460881 | 101460131 | 101461631 | CUX1 | 1 |
| chr16 | 27413483 | 27412733 | 27414233 | IL21R | 1 | chr7 | 25164980 | 25164230 | 25165730 | CYCS | −1 |
| chr16 | 27438578 | 27437828 | 27439328 | IL21R | 1 | chr7 | 108210012 | 108209262 | 108210762 | DNAJB9 | 1 |
| chr16 | 27325234 | 27324484 | 27325984 | IL4R | 1 | chr7 | 36192758 | 36192008 | 36193508 | EEPD1 | 1 |
| chr16 | 28996147 | 28995397 | 28996897 | LAT | 1 | chr7 | 2394501 | 2393751 | 2395251 | EIF3B | 1 |
| chr16 | 28996386 | 28995636 | 28997136 | LAT | 1 | chr7 | 143105985 | 143105235 | 143106735 | EPHA1 | −1 |
| chr16 | 79634611 | 79633861 | 79635361 | MAF | −1 | chr7 | 148581370 | 148580620 | 148582120 | EZH2 | −1 |
| chr16 | 1756184 | 1755434 | 1756934 | MAPK8IP3 | 1 | chr7 | 121036388 | 121035638 | 121037138 | FAM3C | −1 |
| chr16 | 29817917 | 29817167 | 29818667 | MAZ | 1 | chr7 | 5632454 | 5631704 | 5633204 | FSCN1 | 1 |
| chr16 | 55512883 | 55512133 | 55513633 | MMP2 | 1 | chr7 | 150413645 | 150412895 | 150414395 | GIMAP1 | 1 |
| chr16 | 23607612 | 23606862 | 23608362 | NDUFAB1 | −1 | chr7 | 150434436 | 150433686 | 150435186 | GIMAP5 | 1 |
| chr16 | 50731050 | 50730300 | 50731800 | NOD2 | 1 | chr7 | 150329473 | 150328723 | 150330223 | GIMAP6 | −1 |
| chr16 | 56485287 | 56484537 | 56486037 | NUDT21 | −1 | chr7 | 150147718 | 150146968 | 150148468 | GIMAP8 | 1 |
| chr16 | 81812863 | 81812113 | 81813613 | PLCG2 | 1 | chr7 | 8008425 | 8007675 | 8009175 | GLCCI1 | 1 |
| chr16 | 23847345 | 23846595 | 23848095 | PRKCB | 1 | chr7 | 37723475 | 37722725 | 37724225 | GPR141 | 1 |
| chr16 | 28937073 | 28936323 | 28937823 | RABEP2 | −1 | chr7 | 50800050 | 50799300 | 50800800 | GRB10 | −1 |
| chr16 | 325929 | 325179 | 326679 | RGS11 | −1 | chr7 | 50772998 | 50772248 | 50773748 | GRB10 | −1 |
| chr16 | 89627119 | 89626369 | 89627869 | RPL13 | 1 | chr7 | 50861159 | 50860409 | 50861909 | GRB10 | −1 |
| chr16 | 89627064 | 89626314 | 89627814 | RPL13 | 1 | chr7 | 65447301 | 65446551 | 65448051 | GUSB | −1 |
| chr16 | 66959547 | 66958797 | 66960297 | RRAD | −1 | chr7 | 106809460 | 106808710 | 106810210 | HBP1 | 1 |
| chr16 | 29910868 | 29910118 | 29911618 | SEZ6L2 | −1 | chr7 | 106809405 | 106808655 | 106810155 | HBP1 | 1 |
| chr16 | 11762270 | 11761520 | 11763020 | SNN | 1 | chr7 | 18535926 | 18535176 | 18536676 | HDAC9 | 1 |
| chr16 | 12070595 | 12069845 | 12071345 | SNX29 | 1 | chr7 | 81399514 | 81398764 | 81400264 | HGF | −1 |
| chr16 | 11350036 | 11349286 | 11350786 | SOCS1 | −1 | chr7 | 23510086 | 23509336 | 23510836 | IGF2BP3 | −1 |
| chr16 | 1031808 | 1031058 | 1032558 | SOX8 | 1 | chr7 | 50348318 | 50347568 | 50349068 | IKZF1 | 1 |
| chr16 | 29674580 | 29673830 | 29675330 | SPN | 1 | chr7 | 22765503 | 22764753 | 22766253 | IL6 | 1 |
| chr16 | 2802330 | 2801580 | 2803080 | SRRM2 | 1 | chr7 | 6523873 | 6523123 | 6524623 | KDELR2 | −1 |
| chr16 | 19179571 | 19178821 | 19180321 | SYT17 | 1 | chr7 | 107643700 | 107642950 | 107644450 | LAMB1 | −1 |
| chr16 | 84220617 | 84219867 | 84221367 | TAF1C | −1 | chr7 | 2559476 | 2558726 | 2560226 | LFNG | 1 |
| chr16 | 12058964 | 12058214 | 12059714 | TNFRSF17 | 1 | chr7 | 2552162 | 2551412 | 2552912 | LFNG | 1 |
| chr16 | 1290697 | 1289947 | 1291447 | TPSAB1 | 1 | chr7 | 2557496 | 2556746 | 2558246 | LFNG | 1 |
| chr16 | 1358881 | 1358131 | 1359631 | UBE2I | 1 | chr7 | 99699563 | 99698813 | 99700313 | MCM7 | −1 |
| chr16 | 70835064 | 70834314 | 70835814 | VAC14 | −1 | chr7 | 114562209 | 114561459 | 114562959 | MDFIC | 1 |
| chr16 | 89787952 | 89787202 | 89788702 | ZNF276 | 1 | chr7 | 105925638 | 105924888 | 105926388 | NAMPT | −1 |
| chr16 | 89787392 | 89786642 | 89788142 | ZNF276 | 1 | chr7 | 74188309 | 74187559 | 74189059 | NCF1 | 1 |
| chr16 | 2059763 | 2059013 | 2060513 | ZNF598 | −1 | chr7 | 10979883 | 10979133 | 10980633 | NDUFA4 | −1 |
| chr16 | 30418618 | 30417868 | 30419368 | ZNF771 | 1 | chr7 | 108096826 | 108096076 | 108097576 | NRCAM | −1 |
| chr17 | 67138015 | 67137265 | 67138765 | ABCA6 | −1 | chr7 | 102073996 | 102073246 | 102074746 | ORAI2 | 1 |
| chr17 | 76183443 | 76182693 | 76184193 | AFMID | 1 | chr7 | 154794794 | 154794044 | 154795544 | PAXIP1 | −1 |
| chr17 | 36584662 | 36583912 | 36585412 | ARHGAP23 | 1 | chr7 | 94285637 | 94284887 | 94286387 | PEG10 | 1 |
| chr17 | 12692856 | 12692106 | 12693606 | ARHGAP44 | 1 | chr7 | 94285681 | 94284931 | 94286431 | PEG10 | 1 |
| chr17 | 3867585 | 3866835 | 3868335 | ATP2A3 | −1 | chr7 | 99971068 | 99970318 | 99971818 | PILRA | 1 |
| chr17 | 76210267 | 76209517 | 76211017 | BIRC5 | 1 | chr7 | 99933737 | 99932987 | 99934487 | PILRB | 1 |
| chr17 | 79212891 | 79212141 | 79213641 | C17orf56 | −1 | chr7 | 44163107 | 44162357 | 44163857 | POLD2 | −1 |
| chr17 | 42264085 | 42263335 | 42264835 | C17orf65 | −1 | chr7 | 44163169 | 44162419 | 44163919 | POLD2 | −1 |
| chr17 | 5342475 | 5341725 | 5343225 | C1QBP | −1 | chr7 | 95064510 | 95063760 | 95065260 | PON2 | −1 |
| chr17 | 40575535 | 40574785 | 40576285 | CAVIN1 | −1 | chr7 | 94536949 | 94536199 | 94537699 | PPP1R9A | 1 |
| chr17 | 32683471 | 32682721 | 32684221 | CCL13 | 1 | chr7 | 94539209 | 94538459 | 94539959 | PPP1R9A | 1 |
| chr17 | 32582304 | 32581554 | 32583054 | CCL2 | 1 | chr7 | 42971822 | 42971072 | 42972572 | PSMA2 | −1 |
| chr17 | 34345005 | 34344255 | 34345755 | CCL23 | −1 | chr7 | 6414170 | 6413420 | 6414920 | RAC1 | 1 |
| chr17 | 34417487 | 34416737 | 34418237 | CCL3 | −1 | chr7 | 45197390 | 45196640 | 45198140 | RAMP3 | 1 |
| chr17 | 34207512 | 34206762 | 34208262 | CCL5 | −1 | chr7 | 54827667 | 54826917 | 54828417 | SEC61G | −1 |
| chr17 | 38721724 | 38720974 | 38722474 | CCR7 | −1 | chr7 | 80548667 | 80547917 | 80549417 | SEMA3C | −1 |
| chr17 | 72462555 | 72461805 | 72463305 | CD300A | 1 | chr7 | 94285436 | 94284686 | 94286186 | SGCE | −1 |
| chr17 | 7482785 | 7482035 | 7483535 | CD68 | 1 | chr7 | 101928405 | 101927655 | 101929155 | SH2B2 | 1 |
| chr17 | 80275478 | 80274728 | 80276228 | CD7 | −1 | chr7 | 141438407 | 141437657 | 141439157 | SSBP1 | 1 |
| chr17 | 62009696 | 62008946 | 62010446 | CD79B | −1 | chr7 | 141438120 | 141437370 | 141438870 | SSBP1 | 1 |
| chr17 | 73997419 | 73996669 | 73998169 | CDK3 | 1 | chr7 | 99775186 | 99774436 | 99775936 | STAG3 | 1 |
| chr17 | 30813637 | 30812887 | 30814387 | CDK5R1 | 1 | chr7 | 89783689 | 89782939 | 89784439 | STEAP1 | 1 |
| chr17 | 46048407 | 46047657 | 46049157 | CDK5RAP3 | 1 | chr7 | 89841000 | 89840250 | 89841750 | STEAP2 | 1 |
| chr17 | 72983727 | 72982977 | 72984477 | CDR2L | 1 | chr7 | 89841173 | 89840423 | 89841923 | STEAP2 | 1 |
| chr17 | 16256813 | 16256063 | 16257563 | CENPV | −1 | chr7 | 89843133 | 89842383 | 89843883 | STEAP2 | 1 |
| chr17 | 6983626 | 6982876 | 6984376 | CLEC10A | −1 | chr7 | 43622664 | 43621914 | 43623414 | STK17A | 1 |
| chr17 | 40950810 | 40950060 | 40951560 | CNTD1 | 1 | chr7 | 40174575 | 40173825 | 40175325 | SUGCT | 1 |
| chr17 | 48278993 | 48278243 | 48279743 | COL1A1 | −1 | chr7 | 45151313 | 45150563 | 45152063 | TBRG4 | −1 |
| chr17 | 5372380 | 5371630 | 5373130 | DHX33 | −1 | chr7 | 93520303 | 93519553 | 93521053 | TFPI2 | −1 |
| chr17 | 41856356 | 41855606 | 41857106 | DUSP3 | −1 | chr7 | 75623992 | 75623242 | 75624742 | TMEM120A | −1 |
| chr17 | 4854390 | 4853640 | 4855140 | ENO3 | 1 | chr7 | 65670186 | 65669436 | 65670936 | TPST1 | 1 |
| chr17 | 29641130 | 29640380 | 29641880 | EVI2B | −1 | chr7 | 100728720 | 100727970 | 100729470 | TRIM56 | 1 |
| chr17 | 10101868 | 10101118 | 10102618 | GAS7 | −1 | chr7 | 2671585 | 2670835 | 2672335 | TTYH3 | 1 |
| chr17 | 9862766 | 9862016 | 9863516 | GAS7 | −1 | chr7 | 55640218 | 55639468 | 55640968 | VOPP1 | −1 |
| chr17 | 9929623 | 9928873 | 9930373 | GAS7 | −1 | chr7 | 138720775 | 138720025 | 138721525 | ZC3HAV1L | −1 |
| chr17 | 9940064 | 9939314 | 9940814 | GAS7 | −1 | chr7 | 149321881 | 149321131 | 149322631 | ZNF767 | −1 |
| chr17 | 4835592 | 4834842 | 4836342 | GP1BA | 1 | chr8 | 38854505 | 38853755 | 38855255 | ADAM9 | 1 |
| chr17 | 3627211 | 3626461 | 3627961 | GSG2 | 1 | chr8 | 1772142 | 1771392 | 1772892 | ARHGEF10 | 1 |
| chr17 | 1959604 | 1958854 | 1960354 | HIC1 | 1 | chr8 | 11351510 | 11350760 | 11352260 | BLK | 1 |
| chr17 | 1958392 | 1957642 | 1959142 | HIC1 | 1 | chr8 | 27472548 | 27471798 | 27473298 | CLU | −1 |
| chr17 | 46623441 | 46622691 | 46624191 | HOXB2 | −1 | chr8 | 87526656 | 87525906 | 87527406 | CPNE3 | 1 |
| chr17 | 3704537 | 3703787 | 3705287 | ITGAE | −1 | chr8 | 11725738 | 11724988 | 11726488 | CTSB | −1 |
| chr17 | 43025079 | 43024329 | 43025829 | KIF18B | −1 | chr8 | 142138720 | 142137970 | 142139470 | DENND3 | 1 |
| chr17 | 81037567 | 81036817 | 81038317 | METRNL | 1 | chr8 | 29208185 | 29207435 | 29208935 | DUSP4 | −1 |
| chr17 | 55927417 | 55926667 | 55928167 | MRPS23 | −1 | chr8 | 86089622 | 86088872 | 86090372 | E2F5 | 1 |
| chr17 | 55333895 | 55333145 | 55334645 | MSI2 | 1 | chr8 | 86099909 | 86099159 | 86100659 | E2F5 | 1 |
| chr17 | 73598598 | 73597848 | 73599348 | MYO15B | 1 | chr8 | 82395498 | 82394748 | 82396248 | FABP4 | −1 |
| chr17 | 49230937 | 49230187 | 49231687 | NME1 | 1 | chr8 | 82192598 | 82191848 | 82193348 | FABP5 | 1 |
| chr17 | 49230951 | 49230201 | 49231701 | NME2 | 1 | chr8 | 124553446 | 124552696 | 124554196 | FBXO32 | −1 |
| chr17 | 883010 | 882260 | 883760 | NXN | −1 | chr8 | 38325363 | 38324613 | 38326113 | FGFR1 | −1 |
| chr17 | 45899136 | 45898386 | 45899886 | OSBPL7 | −1 | chr8 | 38326352 | 38325602 | 38327102 | FGFR1 | −1 |
| chr17 | 3599698 | 3598948 | 3600448 | P2RX5 | −1 | chr8 | 95274573 | 95273823 | 95275323 | GEM | −1 |
| chr17 | 79818570 | 79817820 | 79819320 | P4HB | −1 | chr8 | 63951730 | 63950980 | 63952480 | GGH | −1 |
| chr17 | 53828340 | 53827590 | 53829090 | PCTP | 1 | chr8 | 42128820 | 42128070 | 42129570 | IKBKB | 1 |
| chr17 | 53828465 | 53827715 | 53829215 | PCTP | 1 | chr8 | 79717758 | 79717008 | 79718508 | IL7 | −1 |
| chr17 | 62407083 | 62406333 | 62407833 | PECAM1 | −1 | chr8 | 23261753 | 23261003 | 23262503 | LOXL2 | −1 |
| chr17 | 4852309 | 4851559 | 4853059 | PFN1 | −1 | chr8 | 105601234 | 105600484 | 105601984 | LRP12 | −1 |
| chr17 | 7142825 | 7142075 | 7143575 | PHF23 | −1 | chr8 | 56792372 | 56791622 | 56793122 | LYN | 1 |
| chr17 | 15165906 | 15165156 | 15166656 | PMP22 | −1 | chr8 | 55014577 | 55013827 | 55015327 | LYPLA1 | −1 |
| chr17 | 2699732 | 2698982 | 2700482 | RAP1GAP2 | 1 | chr8 | 48873496 | 48872746 | 48874246 | MCM4 | 1 |
| chr17 | 38334242 | 38333492 | 38334992 | RAPGEFL1 | 1 | chr8 | 67525484 | 67524734 | 67526234 | MYBL1 | −1 |
| chr17 | 17399707 | 17398957 | 17400457 | RASD1 | −1 | chr8 | 128748330 | 128747580 | 128749080 | MYC | 1 |
| chr17 | 33416338 | 33415588 | 33417088 | RFFL | −1 | chr8 | 82024303 | 82023553 | 82025053 | PAG1 | −1 |
| chr17 | 33390759 | 33390009 | 33391509 | RFFL | −1 | chr8 | 42065242 | 42064492 | 42065992 | PLAT | −1 |
| chr17 | 41150494 | 41149744 | 41151244 | RPL27 | 1 | chr8 | 96146032 | 96145282 | 96146782 | PLEKHF2 | 1 |
| chr17 | 55055466 | 55054716 | 55056216 | SCPEP1 | 1 | chr8 | 38126738 | 38125988 | 38127488 | PLPP5 | −1 |
| chr17 | 75084831 | 75084081 | 75085581 | SEC14L1 | 1 | chr8 | 101162812 | 101162062 | 101163562 | POLR2K | 1 |
| chr17 | 80291874 | 80291124 | 80292624 | SECTM1 | −1 | chr8 | 22298649 | 22297899 | 22299399 | PPP3CC | 1 |
| chr17 | 80191563 | 80190813 | 80192313 | SLC16A3 | 1 | chr8 | 142011257 | 142010507 | 142012007 | PTK2 | −1 |
| chr17 | 33570055 | 33569305 | 33570805 | SLFN5 | 1 | chr8 | 142402093 | 142401343 | 142402843 | PTP4A3 | 1 |
| chr17 | 2207065 | 2206315 | 2207815 | SMG6 | −1 | chr8 | 38458775 | 38458025 | 38459525 | RNF5P1 | −1 |
| chr17 | 1992055 | 1991305 | 1992805 | SMG6 | −1 | chr8 | 74206133 | 74205383 | 74206883 | RPL7 | −1 |
| chr17 | 2140141 | 2139391 | 2140891 | SMG6 | −1 | chr8 | 27491698 | 27490948 | 27492448 | SCARA3 | 1 |
| chr17 | 2169493 | 2168743 | 2170243 | SMG6 | −1 | chr8 | 57233241 | 57232491 | 57233991 | SDR16C5 | −1 |
| chr17 | 46184920 | 46184170 | 46185670 | SNX11 | 1 | chr8 | 134072603 | 134071853 | 134073353 | SLA | −1 |
| chr17 | 46184946 | 46184196 | 46185696 | SNX11 | 1 | chr8 | 134115310 | 134114560 | 134116060 | SLA | −1 |
| chr17 | 26926297 | 26925547 | 26927047 | SPAG5 | −1 | chr8 | 67834784 | 67834034 | 67835534 | SNORD87 | −1 |
| chr17 | 74733415 | 74732665 | 74734165 | SRSF2 | −1 | chr8 | 70378859 | 70378109 | 70379609 | SULF1 | 1 |
| chr17 | 40540586 | 40539836 | 40541336 | STAT3 | −1 | chr8 | 70405027 | 70404277 | 70405777 | SULF1 | 1 |
| chr17 | 40439565 | 40438815 | 40440315 | STAT5A | 1 | chr8 | 110704020 | 110703270 | 110704770 | SYBU | −1 |
| chr17 | 45810610 | 45809860 | 45811360 | TBX21 | 1 | chr8 | 110655419 | 110654669 | 110656169 | SYBU | −1 |
| chr17 | 27405875 | 27405125 | 27406625 | TIAF1 | −1 | chr8 | 110657051 | 110656301 | 110657801 | SYBU | −1 |
| chr17 | 76921469 | 76920719 | 76922219 | TIMP2 | −1 | chr8 | 110657822 | 110657072 | 110658572 | SYBU | −1 |
| chr17 | 76183314 | 76182564 | 76184064 | TK1 | −1 | chr8 | 110661132 | 110660382 | 110661882 | SYBU | −1 |
| chr17 | 26646121 | 26645371 | 26646871 | TMEM97 | 1 | chr8 | 110693234 | 110692484 | 110693984 | SYBU | −1 |
| chr17 | 16875402 | 16874652 | 16876152 | TNFRSF13B | −1 | chr8 | 38644722 | 38643972 | 38645472 | TACC1 | 1 |
| chr17 | 7452471 | 7451721 | 7453221 | TNFSF12 | 1 | chr8 | 38585703 | 38584953 | 38586453 | TACC1 | 1 |
| chr17 | 7461914 | 7461164 | 7462664 | TNFSF13 | 1 | chr8 | 54935089 | 54934339 | 54935839 | TCEA1 | −1 |
| chr17 | 38574202 | 38573452 | 38574952 | TOP2A | −1 | chr8 | 74888444 | 74887694 | 74889194 | TMEM70 | 1 |
| chr17 | 7590856 | 7590106 | 7591606 | TP53 | −1 | chr8 | 22926692 | 22925942 | 22927442 | TNFRSF10B | −1 |
| chr17 | 3461099 | 3460349 | 3461849 | TRPV3 | −1 | chr8 | 60031767 | 60031017 | 60032517 | TOX | −1 |
| chr17 | 40761694 | 40760944 | 40762444 | TUBG1 | 1 | chr8 | 80993051 | 80992301 | 80993801 | TPD52 | −1 |
| chr17 | 19771230 | 19770480 | 19771980 | ULK2 | −1 | chr8 | 56014949 | 56014199 | 56015699 | XKR4 | 1 |
| chr17 | 80332153 | 80331403 | 80332903 | UTS2R | 1 | chr8 | 101965211 | 101964461 | 101965961 | YWHAZ | −1 |
| chr17 | 57784826 | 57784076 | 57785576 | VMP1 | 1 | chr8 | 101962799 | 101962049 | 101963549 | YWHAZ | −1 |
| chr17 | 40215075 | 40214325 | 40215825 | ZNF385C | −1 | chr8 | 101963560 | 101962810 | 101964310 | YWHAZ | −1 |
| chr18 | 60987361 | 60986611 | 60988111 | BCL2 | −1 | chr8 | 101964357 | 101963607 | 101965107 | YWHAZ | −1 |
| chr18 | 5544241 | 5543491 | 5544991 | EPB41L3 | −1 | chr8 | 101965623 | 101964873 | 101966373 | YWHAZ | −1 |
| chr18 | 30353025 | 30352275 | 30353775 | KLHL14 | −1 | chr8 | 102217515 | 102216765 | 102218265 | ZNF706 | −1 |
| chr18 | 56338618 | 56337868 | 56339368 | MALT1 | 1 | chr9 | 129885162 | 129884412 | 129885912 | ANGPTL2 | −1 |
| chr18 | 48723690 | 48722940 | 48724440 | MEX3C | −1 | chr9 | 75772570 | 75771820 | 75773320 | ANXA1 | 1 |
| chr18 | 77160336 | 77159586 | 77161086 | NFATC1 | 1 | chr9 | 94124195 | 94123445 | 94124945 | AUH | −1 |
| chr18 | 21977823 | 21977073 | 21978573 | OSBPL1A | −1 | chr9 | 116111821 | 116111071 | 116112571 | BSPRY | 1 |
| chr18 | 21852196 | 21851446 | 21852946 | OSBPL1A | −1 | chr9 | 77643310 | 77642560 | 77644060 | CARNMT1 | −1 |
| chr18 | 21891470 | 21890720 | 21892220 | OSBPL1A | −1 | chr9 | 34691274 | 34690524 | 34692024 | CCL19 | −1 |
| chr18 | 57567180 | 57566430 | 57567930 | PMAIP1 | 1 | chr9 | 5450525 | 5449775 | 5451275 | CD274 | 1 |
| chr18 | 43652226 | 43651476 | 43652976 | PSTPIP2 | −1 | chr9 | 35619539 | 35618789 | 35620289 | CD72 | −1 |
| chr18 | 52495430 | 52494680 | 52496180 | RAB27B | 1 | chr9 | 123342436 | 123341686 | 123343186 | CDK5RAP2 | −1 |
| chr18 | 9708162 | 9707412 | 9708912 | RAB31 | 1 | chr9 | 135937365 | 135936615 | 135938115 | CEL | 1 |
| chr18 | 56806709 | 56805959 | 56807459 | SEC11C | 1 | chr9 | 131182759 | 131182009 | 131183509 | CERCAM | 1 |
| chr18 | 46477081 | 46476331 | 46477831 | SMAD7 | −1 | chr9 | 91926113 | 91925363 | 91926863 | CKS2 | 1 |
| chr18 | 46469177 | 46468427 | 46469927 | SMAD7 | −1 | chr9 | 34126771 | 34126021 | 34127521 | DCAF12 | −1 |
| chr18 | 46475703 | 46474953 | 46476453 | SMAD7 | −1 | chr9 | 130617035 | 130616285 | 130617785 | ENG | −1 |
| chr18 | 53303185 | 53302435 | 53303935 | TCF4 | −1 | chr9 | 132805441 | 132804691 | 132806191 | FNBP1 | −1 |
| chr18 | 52969852 | 52969102 | 52970602 | TCF4 | −1 | chr9 | 101569981 | 101569231 | 101570731 | GALNT12 | 1 |
| chr18 | 52989090 | 52988340 | 52989840 | TCF4 | −1 | chr9 | 89562104 | 89561354 | 89562854 | GAS1 | −1 |
| chr18 | 53071226 | 53070476 | 53071976 | TCF4 | −1 | chr9 | 6645650 | 6644900 | 6646400 | GLDC | −1 |
| chr18 | 53089723 | 53088973 | 53090473 | TCF4 | −1 | chr9 | 80263223 | 80262473 | 80263973 | GNA14 | −1 |
| chr18 | 53178000 | 53177250 | 53178750 | TCF4 | −1 | chr9 | 80646374 | 80645624 | 80647124 | GNAQ | −1 |
| chr18 | 53255860 | 53255110 | 53256610 | TCF4 | −1 | chr9 | 114423615 | 114422865 | 114424365 | GNG10 | 1 |
| chr18 | 53257045 | 53256295 | 53257795 | TCF4 | −1 | chr9 | 124062071 | 124061321 | 124062821 | GSN | 1 |
| chr18 | 3451675 | 3450925 | 3452425 | TGIF1 | 1 | chr9 | 124030379 | 124029629 | 124031129 | GSN | 1 |
| chr18 | 12308058 | 12307308 | 12308808 | TUBB6 | 1 | chr9 | 124048358 | 124047608 | 124049108 | GSN | 1 |
| chr18 | 657604 | 656854 | 658354 | TYMS | 1 | chr9 | 4985245 | 4984495 | 4985995 | JAK2 | 1 |
| chr18 | 60190240 | 60189490 | 60190990 | ZCCHC2 | 1 | chr9 | 470291 | 469541 | 471041 | KANK1 | 1 |
| chr19 | 17414282 | 17413532 | 17415032 | ABHD8 | −1 | chr9 | 504694 | 503944 | 505444 | KANK1 | 1 |
| chr19 | 6887583 | 6886833 | 6888333 | ADGRE1 | 1 | chr9 | 706805 | 706055 | 707555 | KANK1 | 1 |
| chr19 | 926000 | 925250 | 926750 | ARID3A | 1 | chr9 | 115249127 | 115248377 | 115249877 | KIAA1958 | 1 |
| chr19 | 41725108 | 41724358 | 41725858 | AXL | 1 | chr9 | 139637411 | 139636661 | 139638161 | LCN10 | −1 |
| chr19 | 572565 | 571815 | 573315 | BSG | 1 | chr9 | 113800323 | 113799573 | 113801073 | LPAR1 | −1 |
| chr19 | 47104493 | 47103743 | 47105243 | CALM3 | 1 | chr9 | 127020242 | 127019492 | 127020992 | NEK6 | 1 |
| chr19 | 13049421 | 13048671 | 13050171 | CALR | 1 | chr9 | 94186144 | 94185394 | 94186894 | NFIL3 | −1 |
| chr19 | 35820090 | 35819340 | 35820840 | CD22 | 1 | chr9 | 139440314 | 139439564 | 139441064 | NOTCH1 | −1 |
| chr19 | 8373239 | 8372489 | 8373989 | CD320 | −1 | chr9 | 127533556 | 127532806 | 127534306 | NR6A1 | −1 |
| chr19 | 49838657 | 49837907 | 49839407 | CD37 | 1 | chr9 | 37034103 | 37033353 | 37034853 | PAX5 | −1 |
| chr19 | 42381190 | 42380440 | 42381940 | CD79A | 1 | chr9 | 5510570 | 5509820 | 5511320 | PDCD1LG2 | 1 |
| chr19 | 14492256 | 14491506 | 14493006 | CD97 | 1 | chr9 | 112260586 | 112259836 | 112261336 | PTPN3 | −1 |
| chr19 | 45174994 | 45174244 | 45175744 | CEACAM19 | 1 | chr9 | 112180063 | 112179313 | 112180813 | PTPN3 | −1 |
| chr19 | 43099082 | 43098332 | 43099832 | CEACAM8 | −1 | chr9 | 112191323 | 112190573 | 112192073 | PTPN3 | −1 |
| chr19 | 14693935 | 14693185 | 14694685 | CLEC17A | 1 | chr9 | 112213664 | 112212914 | 112214414 | PTPN3 | −1 |
| chr19 | 6464294 | 6463544 | 6465044 | CRB3 | 1 | chr9 | 135996563 | 135995813 | 135997313 | RALGDS | −1 |
| chr19 | 47164395 | 47163645 | 47165145 | DACT3 | −1 | chr9 | 136024588 | 136023838 | 136025338 | RALGDS | −1 |
| chr19 | 6481819 | 6481069 | 6482569 | DENND1C | −1 | chr9 | 125660389 | 125659639 | 125661139 | RC3H2 | −1 |
| chr19 | 12992282 | 12991532 | 12993032 | DNASE2 | −1 | chr9 | 4792869 | 4792119 | 4793619 | RCL1 | 1 |
| chr19 | 4229495 | 4228745 | 4230245 | EBI3 | 1 | chr9 | 127624240 | 127623490 | 127624990 | RPL35 | −1 |
| chr19 | 17634110 | 17633360 | 17634860 | FAM129C\|NIB | 1 | chr9 | 136215069 | 136214319 | 136215819 | RPL7A | 1 |
| chr19 | 7767032 | 7766282 | 7767782 | FCER2 | −1 | chr9 | 91611421 | 91610671 | 91612171 | S1PR3 | 1 |
| chr19 | 7764365 | 7763615 | 7765115 | FCER2 | −1 | chr9 | 115983842 | 115983092 | 115984592 | SLC31A1 | 1 |
| chr19 | 49977818 | 49977068 | 49978568 | FLT3LG | 1 | chr9 | 115913222 | 115912472 | 115913972 | SLC31A2 | 1 |
| chr19 | 49977465 | 49976715 | 49978215 | FLT3LG | 1 | chr9 | 2021945 | 2021195 | 2022695 | SMARCA2 | 1 |
| chr19 | 52255146 | 52254396 | 52255896 | FPR1 | −1 | chr9 | 126030855 | 126030105 | 126031605 | STRBP | −1 |
| chr19 | 49468558 | 49467808 | 49469308 | FTL | 1 | chr9 | 126030843 | 126030093 | 126031593 | STRBP | −1 |
| chr19 | 35645676 | 35644926 | 35646426 | FXYD5 | 1 | chr9 | 95820989 | 95820239 | 95821739 | SUSD3 | 1 |
| chr19 | 35645624 | 35644874 | 35646374 | FXYD5 | 1 | chr9 | 93564069 | 93563319 | 93564819 | SYK | 1 |
| chr19 | 35645844 | 35645094 | 35646594 | FXYD5 | 1 | chr9 | 71820078 | 71819328 | 71820828 | TJP2 | 1 |
| chr19 | 1401569 | 1400819 | 1402319 | GAMT | −1 | chr9 | 71736179 | 71735429 | 71736929 | TJP2 | 1 |
| chr19 | 46171502 | 46170752 | 46172252 | GIPR | 1 | chr9 | 71788970 | 71788220 | 71789720 | TJP2 | 1 |
| chr19 | 47137942 | 47137192 | 47138692 | GNG8 | −1 | chr9 | 71819926 | 71819176 | 71820676 | TJP2 | 1 |
| chr19 | 34855645 | 34854895 | 34856395 | GPI | 1 | chr9 | 84304220 | 84303470 | 84304970 | TLE1 | −1 |
| chr19 | 34856031 | 34855281 | 34856781 | GPI | 1 | chr9 | 82186688 | 82185938 | 82187438 | TLE4 | 1 |
| chr19 | 35771619 | 35770869 | 35772369 | HAMP | 1 | chr9 | 100263921 | 100263171 | 100264671 | TMOD1 | 1 |
| chr19 | 36393382 | 36392632 | 36394132 | HCST | 1 | chr9 | 100263461 | 100262711 | 100264211 | TMOD1 | 1 |
| chr19 | 10381511 | 10380761 | 10382261 | ICAM1 | 1 | chr9 | 117692697 | 117691947 | 117693447 | TNFSF8 | −1 |
| chr19 | 18284477 | 18283727 | 18285227 | IFI30 | 1 | chr9 | 123691047 | 123690297 | 123691797 | TRAF1 | −1 |
| chr19 | 50432796 | 50432046 | 50433546 | IL4I1 | −1 | chr9 | 15307358 | 15306608 | 15308108 | TTC39B | −1 |
| chr19 | 50400147 | 50399397 | 50400897 | IL4I1 | −1 | chr9 | 15250198 | 15249448 | 15250948 | TTC39B | −1 |
| chr19 | 10764937 | 10764187 | 10765687 | ILF3 | 1 | chr9 | 113018920 | 113018170 | 113019670 | TXN | −1 |
| chr19 | 17958841 | 17958091 | 17959591 | JAK3 | −1 | chr9 | 33817565 | 33816815 | 33818315 | UBE2R2 | 1 |
| chr19 | 16435628 | 16434878 | 16436378 | KLF2 | 1 | chr9 | 136857432 | 136856682 | 136858182 | VAV2 | −1 |
| chr19 | 54960065 | 54959315 | 54960815 | LENG8 | 1 | chr9 | 14693469 | 14692719 | 14694219 | ZDHHC21 | −1 |
| chr19 | 55141908 | 55141158 | 55142658 | LILRB1 | 1 | chrX | 147582139 | 147581389 | 147582889 | AFF2 | 1 |
| chr19 | 55128628 | 55127878 | 55129378 | LILRB1 | 1 | chrX | 147800614 | 147799864 | 147801364 | AFF2 | 1 |
| chr19 | 55141967 | 55141217 | 55142717 | LILRB1 | 1 | chrX | 129139164 | 129138414 | 129139914 | BCORL1 | 1 |
| chr19 | 4540486 | 4539736 | 4541236 | LRG1 | −1 | chrX | 100641183 | 100640433 | 100641933 | BTK | −1 |
| chr19 | 18434084 | 18433334 | 18434834 | LSM4 | −1 | chrX | 135730352 | 135729602 | 135731102 | CD40LG | 1 |
| chr19 | 39108564 | 39107814 | 39109314 | MAP4K1 | −1 | chrX | 2609220 | 2608470 | 2609970 | CD99 | 1 |
| chr19 | 19302967 | 19302217 | 19303717 | MEF2B | −1 | chrX | 70838360 | 70837610 | 70839110 | CXCR3 | −1 |
| chr19 | 54372660 | 54371910 | 54373410 | MYADM | 1 | chrX | 37639264 | 37638514 | 37640014 | CYBB | 1 |
| chr19 | 54369610 | 54368860 | 54370360 | MYADM | 1 | chrX | 153991031 | 153990281 | 153991781 | DKC1 | 1 |
| chr19 | 54371136 | 54370386 | 54371886 | MYADM | 1 | chrX | 154251028 | 154250278 | 154251778 | F8 | −1 |
| chr19 | 54372780 | 54372030 | 54373530 | MYADM | 1 | chrX | 119445391 | 119444641 | 119446141 | FAM70A | −1 |
| chr19 | 50706902 | 50706152 | 50707652 | MYH14 | 1 | chrX | 135229559 | 135228809 | 135230309 | FHL1 | 1 |
| chr19 | 8642461 | 8641711 | 8643211 | MYO1F | −1 | chrX | 135228860 | 135228110 | 135229610 | FHL1 | 1 |
| chr19 | 50848005 | 50847255 | 50848755 | NAPSB | −1 | chrX | 135230736 | 135229986 | 135231486 | FHL1 | 1 |
| chr19 | 3366584 | 3365834 | 3367334 | NFIC | 1 | chrX | 135251454 | 135250704 | 135252204 | FHL1 | 1 |
| chr19 | 39390639 | 39389889 | 39391389 | NFKB1B | 1 | chrX | 135251795 | 135251045 | 135252545 | FHL1 | 1 |
| chr19 | 51875969 | 51875219 | 51876719 | NKG7 | −1 | chrX | 135278912 | 135278162 | 135279662 | FHL1 | 1 |
| chr19 | 56348128 | 56347378 | 56348878 | NLRP11 | −1 | chrX | 135279054 | 135278304 | 135279804 | FHL1 | 1 |
| chr19 | 48248779 | 48248029 | 48249529 | NOP53 | 1 | chrX | 153602994 | 153602244 | 153603744 | FLNA | −1 |
| chr19 | 42636579 | 42635829 | 42637329 | POU2F2 | −1 | chrX | 49121288 | 49120538 | 49122038 | FOXP3 | −1 |
| chr19 | 291170 | 290420 | 291920 | PPAP2C | −1 | chrX | 151143151 | 151142401 | 151143901 | GABRE | −1 |
| chr19 | 291435 | 290685 | 292185 | PPAP2C | −1 | chrX | 41583408 | 41582658 | 41584158 | GPR82 | 1 |
| chr19 | 12912673 | 12911923 | 12913423 | PRDX2 | −1 | chrX | 65382391 | 65381641 | 65383141 | HEPH | 1 |
| chr19 | 43853207 | 43852457 | 43853957 | PRG1 | 1 | chrX | 150151759 | 150151009 | 150152509 | HMGB3 | 1 |
| chr19 | 47128375 | 47127625 | 47129125 | PTGIR | −1 | chrX | 153770459 | 153769709 | 153771209 | IKBKG | 1 |
| chr19 | 42463542 | 42462792 | 42464292 | RABAC1 | −1 | chrX | 153775561 | 153774811 | 153776311 | IKBKG | 1 |
| chr19 | 45504688 | 45503938 | 45505438 | RELB | 1 | chrX | 153776061 | 153775311 | 153776811 | IKBKG | 1 |
| chr19 | 10341948 | 10341198 | 10342698 | S1PR2 | −1 | chrX | 117861535 | 117860785 | 117862285 | IL13RA1 | 1 |
| chr19 | 47634120 | 47633370 | 47634870 | SAE1 | 1 | chrX | 153285431 | 153284681 | 153286181 | IRAK1 | −1 |
| chr19 | 47634079 | 47633329 | 47634829 | SAE1 | 1 | chrX | 69509940 | 69509190 | 69510690 | KIF4A | 1 |
| chr19 | 48281829 | 48281079 | 48282579 | SELENOW | 1 | chrX | 140271310 | 140270560 | 140272060 | LDOC1 | −1 |
| chr19 | 51920952 | 51920202 | 51921702 | SIGLEC10 | −1 | chrX | 130964671 | 130963921 | 130965421 | LOC286467 | −1 |
| chr19 | 52150054 | 52149304 | 52150804 | SIGLEC14 | −1 | chrX | 19362011 | 19361261 | 19362761 | PDHA1 | 1 |
| chr19 | 52035057 | 52034307 | 52035807 | SIGLEC6 | −1 | chrX | 46771735 | 46770985 | 46772485 | PHF16 | 1 |
| chr19 | 51628165 | 51627415 | 51628915 | SIGLEC9 | 1 | chrX | 46771867 | 46771117 | 46772617 | PHF16 | 1 |
| chr19 | 11071677 | 11070927 | 11072427 | SMARCA4 | 1 | chrX | 48776301 | 48775551 | 48777051 | PIM2 | −1 |
| chr19 | 46195591 | 46194841 | 46196341 | SNRPD2 | −1 | chrX | 7895484 | 7894734 | 7896234 | PNPLA4 | −1 |
| chr19 | 50922195 | 50921445 | 50922945 | SPIB | 1 | chrX | 7895780 | 7895030 | 7896530 | PNPLA4 | −1 |
| chr19 | 1652604 | 1651854 | 1653354 | TCF3 | −1 | chrX | 37208583 | 37207833 | 37209333 | PRRG1 | 1 |
| chr19 | 3029165 | 3028415 | 3029915 | TLE2 | −1 | chrX | 37208527 | 37207777 | 37209277 | PRRG1 | 1 |
| chr19 | 3029612 | 3028862 | 3030362 | TLE2 | −1 | chrX | 118925606 | 118924856 | 118926356 | RPL39 | −1 |
| chr19 | 3047633 | 3046883 | 3048383 | TLE2 | −1 | chrX | 71497150 | 71496400 | 71497900 | RPS4X | −1 |
| chr19 | 3063105 | 3062355 | 3063855 | TLE5 | −1 | chrX | 23801290 | 23800540 | 23802040 | SAT1 | 1 |
| chr19 | 36036497 | 36035747 | 36037247 | TMEM147 | 1 | chrX | 123480194 | 123479444 | 123480944 | SH2D1A | 1 |
| chr19 | 10491352 | 10490602 | 10492102 | TYK2 | −1 | chrX | 80457442 | 80456692 | 80458192 | SH3BGRL | 1 |
| chr19 | 9938568 | 9937818 | 9939318 | UBL5 | 1 | chrX | 118602363 | 118601613 | 118603113 | SLC25A5 | 1 |
| chr19 | 9938555 | 9937805 | 9939305 | UBL5 | 1 | chrX | 46618490 | 46617740 | 46619240 | SLC9A7 | −1 |
| chr19 | 46010674 | 46009924 | 46011424 | VASP | 1 | chrX | 38080152 | 38079402 | 38080902 | SRPX | −1 |
| chr19 | 50529203 | 50528453 | 50529953 | VRK3 | −1 | chrX | 99899215 | 99898465 | 99899965 | SRPX2 | 1 |
| chr19 | 36195429 | 36194679 | 36196179 | ZBTB32 | 1 | chrX | 99986522 | 99985772 | 99987272 | SYTL4 | −1 |
| chr19 | 44669308 | 44668558 | 44670058 | ZNF226 | 1 | chrX | 99987135 | 99986385 | 99987885 | SYTL4 | −1 |
| chr19 | 44669214 | 44668464 | 44669964 | ZNF226 | 1 | chrX | 47441712 | 47440962 | 47442462 | TIMP1 | 1 |
| chr19 | 44669248 | 44668498 | 44669998 | ZNF226 | 1 | chrX | 12885202 | 12884452 | 12885952 | TLR7 | 1 |
| chr19 | 54024278 | 54023528 | 54025028 | ZNF331 | 1 | chrX | 12924739 | 12923989 | 12925489 | TLR8 | 1 |
| chr19 | 22019010 | 22018260 | 22019760 | ZNF43 | −1 | chrX | 101771712 | 101770962 | 101772462 | TMSB15A | −1 |
| chr19 | 22034870 | 22034120 | 22035620 | ZNF43 | −1 | chrX | 65259949 | 65259199 | 65260699 | VSIG4 | −1 |
| chr2 | 264869 | 264119 | 265619 | ACP1 | 1 | chrX | 37545012 | 37544262 | 37545762 | XK | 1 |
| chr2 | 158731623 | 158730873 | 158732373 | ACVR1 | −1 | chrX | 84499038 | 84498288 | 84499788 | ZNF711 | 1 |
| chr2 | 158732374 | 158731624 | 158733124 | ACVR1 | −1 | chrY | 21154705 | 21153955 | 21155455 | CD24 | −1 |
| chr2 | 100722045 | 100721295 | 100722795 | AFF3 | −1 | chrY | 2559227 | 2558477 | 2559977 | CD99 | 1 |
| chr2 | 100759037 | 100758287 | 100759787 | AFF3 | −1 | chrY | 15016742 | 15015992 | 15017492 | DDX3Y | 1 |
| chr2 | 236402751 | 236402001 | 236403501 | AGAP1 | 1 | chrY | 22737611 | 22736861 | 22738361 | EIF1AY | 1 |
| chr2 | 30144432 | 30143682 | 30145182 | ALK | −1 | chrY | 2709527 | 2708777 | 2710277 | RPS4Y1 | 1 |

TABLE2
Transcription Start Sites for Interrogation to Detect Immune Diseases

| CHR | TSS | Start | Stop | Gene-Symbol | Strand | CHR | TSS | Start | Stop | Gene-Symbol | Strand |
|---|---|---|---|---|---|---|---|---|---|---|---|
| chr1 | 117210314 | 117209564 | 117211064 | IGSF3 | −1 | chr4 | 48271881 | 48271131 | 48272631 | TEC | −1 |
| chr1 | 153589462 | 153588712 | 153590212 | S100A14 | −1 | chr4 | 79697532 | 79696782 | 79698282 | BMP2K | 1 |
| chr1 | 201368736 | 201367986 | 201369486 | LAD1 | −1 | chr4 | 74904406 | 74903656 | 74905156 | CXCL3 | −1 |
| chr1 | 183155423 | 183154673 | 183156173 | LAMC2 | 1 | chr4 | 73434516 | 73433766 | 73435266 | ADAMTS3 | −1 |
| chr1 | 161059389 | 161058639 | 161060139 | PVRL4 | −1 | chr4 | 84256306 | 84255556 | 84257056 | HPSE | −1 |
| chr1 | 156030951 | 156030201 | 156031701 | RAB25 | 1 | chr4 | 84255966 | 84255216 | 84256716 | HPSE | −1 |
| chr1 | 46640759 | 46640009 | 46641509 | TSPAN1 | 1 | chr4 | 84256034 | 84255284 | 84256784 | HPSE | −1 |
| chr11 | 394217 | 393467 | 394967 | PKP3 | 1 | chr4 | 39034041 | 39033291 | 39034791 | TMEM156 | −1 |
| chr11 | 117947804 | 117947054 | 117948554 | TMPRSS4 | 1 | chr4 | 15704573 | 15703823 | 15705323 | BST1 | 1 |
| chr12 | 56473645 | 56472895 | 56474395 | ERBB3 | 1 | chr4 | 74853914 | 74853164 | 74854664 | PPBP | −1 |
| chr12 | 13043716 | 13042966 | 13044466 | GPRC5A | 1 | chr4 | 156298122 | 156297372 | 156298872 | MAP9 | −1 |
| chr12 | 91505608 | 91504858 | 91506358 | LUM | −1 | chr4 | 48136273 | 48135523 | 48137023 | TXK | −1 |
| chr13 | 38172981 | 38172231 | 38173731 | POSTN | −1 | chr4 | 74864496 | 74863746 | 74865246 | CXCL5 | −1 |
| chr16 | 68678739 | 68677989 | 68679489 | CDH3 | 1 | chr4 | 69434245 | 69433495 | 69434995 | UGT2B17 | −1 |
| chr16 | 10674555 | 10673805 | 10675305 | EMP2 | −1 | chr4 | 68424446 | 68423696 | 68425196 | STAP1 | 1 |
| chr16 | 31147083 | 31146333 | 31147833 | PRSS8 | −1 | chr4 | 154622652 | 154621902 | 154623402 | TLR2 | 1 |
| chr17 | 39684560 | 39683810 | 39685310 | KRT19 | −1 | chr4 | 159094202 | 159093452 | 159094952 | FAM198B | −1 |
| chr18 | 29078006 | 29077256 | 29078756 | DSG2 | 1 | chr4 | 159093718 | 159092968 | 159094468 | FAM198B | −1 |
| chr19 | 35607166 | 35606416 | 35607916 | FXYD3 | 1 | chr4 | 144826712 | 144825962 | 144827462 | GYPE | −1 |
| chr19 | 42212537 | 42211787 | 42213287 | CEACAM5 | 1 | chr4 | 123542224 | 123541474 | 123542974 | IL21 | −1 |
| chr19 | 50706902 | 50706152 | 50707652 | MYH14 | 1 | chr4 | 122148621 | 122147871 | 122149371 | TNIP3 | −1 |
| chr19 | 291170 | 290420 | 291920 | PPAP2C | −1 | chr4 | 122085495 | 122084745 | 122086245 | TNIP3 | −1 |
| chr19 | 291435 | 290685 | 292185 | PPAP2C | −1 | chr4 | 122137782 | 122137032 | 122138532 | TNIP3 | −1 |
| chr2 | 47596287 | 47595537 | 47597037 | EPCAM | 1 | chr4 | 38666249 | 38665499 | 38666999 | R11-617D20.1 | −1 |
| chr2 | 190044605 | 190043855 | 190045355 | COL5A2 | −1 | chr4 | 38626196 | 38625446 | 38626946 | R11-617D20.1 | −1 |
| chr2 | 161056812 | 161056062 | 161057562 | ITGB6 | −1 | chr4 | 38666476 | 38665726 | 38667226 | R11-617D20.1 | −1 |
| chr2 | 110874143 | 110873393 | 110874893 | MALL | −1 | chr4 | 38666504 | 38665754 | 38667254 | R11-617D20.1 | −1 |
| chr21 | 18884700 | 18883950 | 18885450 | CXADR | 1 | chr5 | 148206156 | 148205406 | 148206906 | ADRB2 | 1 |
| chr3 | 148939842 | 148939092 | 148940592 | CP | −1 | chr5 | 126366500 | 126365750 | 126367250 | 3-Mar | −1 |
| chr4 | 187645009 | 187644259 | 187645759 | FAT1 | −1 | chr5 | 176326333 | 176325583 | 176327083 | HK3 | −1 |
| chr7 | 73242074 | 73241324 | 73242824 | CLDN4 | 1 | chr5 | 107006596 | 107005846 | 107007346 | EFNA5 | −1 |
| chr7 | 116312459 | 116311709 | 116313209 | MET | 1 | chr5 | 179220987 | 179220237 | 179221737 | LTC4S | 1 |
| chr7 | 16844704 | 16843954 | 16845454 | AGR2 | −1 | chr5 | 132113067 | 132112317 | 132113817 | 8-Sep | −1 |
| chr7 | 55086794 | 55086044 | 55087544 | EGFR | 1 | chr5 | 132113561 | 132112811 | 132114311 | 8-Sep | −1 |
| chr8 | 95653357 | 95652607 | 95654107 | ESRP1 | 1 | chr5 | 158757895 | 158757145 | 158758645 | IL12B | −1 |
| chr8 | 102504660 | 102503910 | 102505410 | GRHL2 | 1 | chr5 | 137225158 | 137224408 | 137225908 | PKD2L2 | 1 |
| chr8 | 120220610 | 120219860 | 120221360 | MAL2 | 1 | chr5 | 66492627 | 66491877 | 66493377 | CD180 | −1 |
| chr8 | 70378859 | 70378109 | 70379609 | SULF1 | 1 | chr5 | 131396222 | 131395472 | 131396972 | IL3 | 1 |
| chr8 | 70405027 | 70404277 | 70405777 | SULF1 | 1 | chr5 | 180326105 | 180325355 | 180326855 | BTNL8 | 1 |
| chr9 | 137533620 | 137532870 | 137534370 | COL5A1 | 1 | chr5 | 55529186 | 55528436 | 55529936 | ANKRD55 | −1 |
| chrX | 3264682 | 3263932 | 3265432 | MXRA5 | −1 | chr5 | 135231516 | 135230766 | 135232266 | IL9 | −1 |
| chr9 | 117880536 | 117879786 | 117881286 | TNC | −1 | chr5 | 132009678 | 132008928 | 132010428 | IL4 | 1 |
| chr10 | 81370701 | 81369951 | 81371451 | SFTPA1 | 1 | chr5 | 22853731 | 22852981 | 22854481 | CDH12 | −1 |
| chr10 | 81320151 | 81319401 | 81320901 | SFTPA2 | −1 | chr5 | 140201361 | 140200611 | 140202111 | PCDHA5 | 1 |
| chr17 | 39780829 | 39780079 | 39781579 | KRT17 | −1 | chr5 | 156607837 | 156607087 | 156608587 | ITK | 1 |
| chr19 | 42947200 | 42946450 | 42947950 | CXCL17 | −1 | chr6 | 155777037 | 155776287 | 155777787 | NOX3 | −1 |
| chr2 | 85895864 | 85895114 | 85896614 | SFTPB | −1 | chr6 | 31582961 | 31582211 | 31583711 | AIF1 | 1 |
| chr20 | 44098346 | 44097596 | 44099096 | WFDC2 | 1 | chr6 | 32784825 | 32784075 | 32785575 | HLA-DOB | −1 |
| chr3 | 190040264 | 190039514 | 190041014 | CLDN1 | −1 | chr6 | 31554977 | 31554227 | 31555727 | LST1 | 1 |
| chr4 | 25657466 | 25656716 | 25658216 | SLC34A2 | 1 | chr6 | 31553955 | 31553205 | 31554705 | LSTI | 1 |
| chr15 | 73976554 | 73975804 | 73977304 | CD276 | 1 | chr6 | 31554475 | 31553725 | 31555225 | LST1 | 1 |
| chr2 | 20425194 | 20424444 | 20425944 | SDC1 | −1 | chr6 | 31539831 | 31539081 | 31540581 | LTA | 1 |
| chr2 | 20424927 | 20424177 | 20425677 | SDC1 | −1 | chr6 | 31560762 | 31560012 | 31561512 | NCR3 | −1 |
| chr5 | 137801179 | 137800429 | 137801929 | EGR1 | 1 | chr6 | 96025419 | 96024669 | 96026169 | MANEA | 1 |
| chr7 | 22765503 | 22764753 | 22766253 | IL6 | 1 | chr6 | 26216872 | 26216122 | 26217622 | HIST1H2BG | −1 |
| chr2 | 96811179 | 96810429 | 96811929 | DUSP2 | −1 | chr6 | 119670926 | 119670176 | 119671676 | MAN1A1 | −1 |
| chr10 | 120938345 | 120937595 | 120939095 | PRDX3 | −1 | chr6 | 26217165 | 26216415 | 26217915 | HIST1H2AE | 1 |
| chr1 | 207495046 | 207494296 | 207495796 | CD55 | 1 | chr6 | 24911195 | 24910445 | 24911945 | FAM65B | −1 |
| chr14 | 96180533 | 96179783 | 96181283 | TCL1A | −1 | chr6 | 24877583 | 24876833 | 24878333 | FAM65B | −1 |
| chr11 | 60223320 | 60222570 | 60224070 | MS4A1 | 1 | chr6 | 133079147 | 133078397 | 133079897 | VNN2 | −1 |
| chr12 | 10183276 | 10182526 | 10184026 | CLEC9A | 1 | chr6 | 10838728 | 10837978 | 10839478 | MAK | −1 |
| chr3 | 46069234 | 46068484 | 46069984 | XCR1 | −1 | chr6 | 133055831 | 133055081 | 133056581 | VNN3 | −1 |
| chr16 | 1290697 | 1289947 | 1291447 | TPSAB1 | 1 | chr6 | 133035188 | 133034438 | 133035938 | VNN1 | −1 |
| chr17 | 6983626 | 6982876 | 6984376 | CLEC10A | −1 | chr6 | 41168932 | 41168182 | 41169682 | TREML2 | −1 |
| chr3 | 46395235 | 46394485 | 46395985 | CCR2 | 1 | chr6 | 28048753 | 28048003 | 28049503 | ZNF165 | 1 |
| chr1 | 111415776 | 111415026 | 111416526 | CD53 | 1 | chr6 | 41254457 | 41253707 | 41255207 | TREM1 | −1 |
| chr1 | 89531043 | 89530293 | 89531793 | GBP1 | −1 | chr6 | 142409936 | 142409186 | 142410686 | NMBR | −1 |
| chr1 | 159796540 | 159795790 | 159797290 | SLAMF8 | 1 | chr6 | 6588341 | 6587591 | 6589091 | LY86 | 1 |
| chr1 | 167487775 | 167487025 | 167488525 | CD247 | −1 | chr6 | 41130914 | 41130164 | 41131664 | TREM2 | −1 |
| chr2 | 136873813 | 136873063 | 136874563 | CXCR4 | −1 | chr6 | 116381921 | 116381171 | 116382671 | FRK | −1 |
| chr2 | 136875725 | 136874975 | 136876475 | CXCR4 | −1 | chr6 | 46761127 | 46760377 | 46761877 | MEP1A | 1 |
| chr2 | 204801471 | 204800721 | 204802221 | ICOS | 1 | chr6 | 49712150 | 49711400 | 49712900 | CRISP3 | −1 |
| chr12 | 6881686 | 6880936 | 6882436 | LAG3 | 1 | chr6 | 49712168 | 49711418 | 49712918 | CRISP3 | −1 |
| chr3 | 114009949 | 114009199 | 114010699 | TIGIT | 1 | chr6 | 46703079 | 46702329 | 46703829 | PLA2G7 | −1 |
| chr5 | 156536725 | 156535975 | 156537475 | HAVCR2 | −1 | chr6 | 27339304 | 27338554 | 27340054 | ZNF204P | −1 |
| chr16 | 56701977 | 56701227 | 56702727 | MT1G | −1 | chr6 | 27343153 | 27342403 | 27343903 | ZNF204P | −1 |
| chr17 | 19290503 | 19289753 | 19291253 | MFAP4 | −1 | chr6 | 96438983 | 96438233 | 96439733 | KRT18P50 | 1 |
| chr2 | 220283099 | 220282349 | 220283849 | DES | 1 | chr6 | 108326772 | 108326022 | 108327522 | RPL3P7 | −1 |
| chr5 | 152871732 | 152870982 | 152872482 | GRIA1 | 1 | chr7 | 140624564 | 140623814 | 140625314 | BRAF | −1 |
| chr5 | 152870083 | 152869333 | 152870833 | GRIA1 | 1 | chr7 | 92463231 | 92462481 | 92463981 | CDK6 | −1 |
| chr5 | 152870856 | 152870106 | 152871606 | GRIA1 | 1 | chr7 | 92465941 | 92465191 | 92466691 | CDK6 | −1 |
| chr6 | 29527702 | 29526952 | 29528452 | UBD | −1 | chr7 | 100493541 | 100492791 | 100494291 | ACHE | −1 |
| chr6 | 41715135 | 41714385 | 41715885 | PGC | −1 | chr7 | 143105985 | 143105235 | 143106735 | EPHA1 | −1 |
| chr7 | 73442119 | 73441369 | 73442869 | ELN | 1 | chr7 | 87936206 | 87935456 | 87936956 | STEAP4 | −1 |
| chr7 | 100808874 | 100808124 | 100809624 | VGF | −1 | chr7 | 87105055 | 87104305 | 87105805 | ABCB4 | −1 |
| chr7 | 99573780 | 99573030 | 99574530 | AZGP1 | −1 | chr7 | 99766373 | 99765623 | 99767123 | GAL3ST4 | −1 |
| chr7 | 142240058 | 142239308 | 142240808 | TRBV9 | −1 | chr7 | 8301911 | 8301161 | 8302661 | ICA1 | −1 |
| chr12 | 71833550 | 71832800 | 71834300 | LGR5 | 1 | chr7 | 37888199 | 37887449 | 37888949 | NME8 | 1 |
| chr12 | 114846247 | 114845497 | 114846997 | TBX5 | −1 | chr7 | 28452144 | 28451394 | 28452894 | CREB5 | 1 |
| chr12 | 114843968 | 114843218 | 114844718 | TBX5 | −1 | chr7 | 28338939 | 28338189 | 28339689 | CREB5 | 1 |
| chr3 | 165555260 | 165554510 | 165556010 | BCHE | −1 | chr7 | 28475233 | 28474483 | 28475983 | CREB5 | 1 |
| chr4 | 186456662 | 186455912 | 186457412 | PDLIM3 | −1 | chr7 | 28725720 | 28724970 | 28726470 | CREB5 | 1 |
| chr12 | 49525178 | 49524428 | 49525928 | TUBA1B | −1 | chr7 | 141695638 | 141694888 | 141696388 | MGAM | 1 |
| chr17 | 8113916 | 8113166 | 8114666 | AURKB | −1 | chr7 | 99816859 | 99816109 | 99817609 | PVR1G | 1 |
| chr20 | 44441215 | 44440465 | 44441965 | UBE2C | 1 | chr8 | 28351773 | 28351023 | 28352523 | FZD3 | 1 |
| chr4 | 174256276 | 174255526 | 174257026 | HMGB2 | −1 | chr8 | 61193971 | 61193221 | 61194721 | CA8 | −1 |
| chr2 | 204732509 | 204731759 | 204733259 | CTLA4 | 1 | chr8 | 72756703 | 72755953 | 72757453 | MSC | −1 |
| chr5 | 54320081 | 54319331 | 54320831 | GZMK | 1 | chr8 | 22960103 | 22959353 | 22960853 | TNFRSF10C | 1 |
| chr5 | 54398476 | 54397726 | 54399226 | GZMA | 1 | chr8 | 81397902 | 81397152 | 81398652 | ZBTB10 | 1 |
| chr4 | 102711764 | 102711014 | 102712514 | BANK1 | 1 | chr8 | 28174649 | 28173899 | 28175399 | PNOC | 1 |
| chr4 | 102734982 | 102734232 | 102735732 | BANK1 | 1 | chr8 | 105352054 | 105351304 | 105352804 | DCSTAMP | 1 |
| chr1 | 158301321 | 158300571 | 158302071 | CD1B | −1 | chr8 | 6795860 | 6795110 | 6796610 | DEFA4 | −1 |
| chr1 | 158259576 | 158258826 | 158260326 | CD1C | 1 | chr8 | 24151553 | 24150803 | 24152303 | ADAM28 | 1 |
| chr1 | 158323540 | 158322790 | 158324290 | CD1E | 1 | chr8 | 24241798 | 24241048 | 24242548 | ADAMDEC1 | 1 |
| chr1 | 203155877 | 203155127 | 203156627 | CHI3L1 | −1 | chr8 | 70747299 | 70746549 | 70748049 | SLCOSA1 | −1 |
| chr1 | 161600996 | 161600246 | 161601746 | FCGR3B | −1 | chr8 | 70745404 | 70744654 | 70746154 | SLCOSA1 | −1 |
| chr1 | 192605275 | 192604525 | 192606025 | RGS13 | 1 | chr8 | 70745625 | 70744875 | 70746375 | SLCOSA1 | −1 |
| chr14 | 25078864 | 25078114 | 25079614 | GZMH | −1 | chr9 | 130679305 | 130678555 | 130680055 | ST6GALNAC4 | −1 |
| chr3 | 151047336 | 151046586 | 151048086 | P2RY13 | −1 | chr9 | 136344259 | 136343509 | 136345009 | SLC2A6 | −1 |
| chr4 | 95264027 | 95263277 | 95264777 | HPGDS | −1 | chr9 | 107526451 | 107525701 | 107527201 | NIPSNAP3B | 1 |
| chr8 | 11351510 | 11350760 | 11352260 | BLK | 1 | chr9 | 77503010 | 77502260 | 77503760 | TRPM6 | −1 |
| chr9 | 137809809 | 137809059 | 137810559 | FCN1 | −1 | chr9 | 77502263 | 77501513 | 77503013 | TRPM6 | −1 |
| chr14 | 72399156 | 72398406 | 72399906 | RGS6 | 1 | chr9 | 77502636 | 77501886 | 77503386 | TRPM6 | −1 |
| chr14 | 72398816 | 72398066 | 72399566 | RGS6 | 1 | chr9 | 35650937 | 35650187 | 35651687 | SIT1 | −1 |
| chr14 | 72399785 | 72399035 | 72400535 | RGS6 | 1 | chr9 | 21207142 | 21206392 | 21207892 | IFNA10 | −1 |
| chr3 | 181429722 | 181428972 | 181430472 | SOX2 | 1 | chr9 | 35619539 | 35618789 | 35620289 | CD72 | −1 |
| chr6 | 138428648 | 138427898 | 138429398 | PERP | −1 | chrX | 16964814 | 16964064 | 16965564 | REPS2 | 1 |
| chr6 | 123100620 | 123099870 | 123101370 | FABP7 | 1 | chrX | 46433219 | 46432469 | 46433969 | CHST7 | 1 |
| chr6 | 64029882 | 64029132 | 64030632 | LGSN | −1 | chrX | 153210143 | 153209393 | 153210893 | RENBP | −1 |
| chr9 | 88714473 | 88713723 | 88715223 | GOLM1 | −1 | chrX | 119445288 | 119444538 | 119446038 | TMEM255A | −1 |
| chrX | 114827819 | 114827069 | 114828569 | PLS3 | 1 | chrX | 105855202 | 105854452 | 105855952 | CXorf57 | 1 |
| chrX | 114795176 | 114794426 | 114795926 | PLS3 | 1 | chrX | 132549518 | 132548768 | 132550268 | GPC4 | −1 |
| chr7 | 27135593 | 27134843 | 27136343 | HOXA1 | −1 | chrX | 154563955 | 154563205 | 154564705 | CLIC2 | −1 |
| chr19 | 51875969 | 51875219 | 51876719 | NKG7 | −1 | chrX | 78200918 | 78200168 | 78201668 | P2RY10 | 1 |
| chr14 | 25103473 | 25102723 | 25104223 | GZMB | −1 | chrX | 12924739 | 12923989 | 12925489 | TLR8 | 1 |
| chr4 | 109089578 | 109088828 | 109090328 | LEF1 | −1 | chrX | 135730352 | 135729602 | 135731102 | CD40LG | 1 |
| chr19 | 41859816 | 41859066 | 41860566 | TGFB1 | −1 | chrX | 12885202 | 12884452 | 12885952 | TLR7 | −1 |
| chr6 | 32407619 | 32406869 | 32408369 | HLA-DRA | 1 | chrX | 47489704 | 47488954 | 47490454 | CFP | −1 |
| chr4 | 78432907 | 78432157 | 78433657 | CXCL13 | 1 | chrX | 47489369 | 47488619 | 47490119 | CFP | 1 |
| chr2 | 65216535 | 65215785 | 65217285 | SLC1A4 | 1 | chrX | 148793738 | 148792988 | 148794488 | MAGEA11 | 1 |
| chr11 | 65265232 | 65264482 | 65265982 | MALATI | 1 | chrX | 148769902 | 148769152 | 148770652 | MAGEA11 | 1 |
| chr19 | 48828582 | 48827832 | 48829332 | EMP3 | 1 | chrX | 48114752 | 48114002 | 48115502 | SSX1 | 1 |
| chr15 | 89181974 | 89181224 | 89182724 | ISG20 | 1 | chrX | 111933143 | 111932393 | 111933893 | HMGB3P30 | 1 |
| chr6 | 159240444 | 159239694 | 159241194 | EZR | −1 | chrX | 111933149 | 111932399 | 111933899 | HMGB3P30 | 1 |
| chr7 | 40174575 | 40173825 | 40175325 | SUGCT | 1 | chr16 | 28943260 | 28942510 | 28944010 | CD19 | 1 |
| chr12 | 8765467 | 8764717 | 8766217 | AICDA | −1 | chr19 | 42381190 | 42380440 | 42381940 | CD79A | 1 |
| chr2 | 204571271 | 204570521 | 204572021 | CD28 | 1 | chr16 | 12058964 | 12058214 | 12059714 | TNFRSF17 | 1 |
| chr10 | 97515409 | 97514659 | 97516159 | ENTPD1 | 1 | chr19 | 49977818 | 49977068 | 49978568 | FLT3LG | 1 |
| chr10 | 97471535 | 97470785 | 97472285 | ENTPD1 | 1 | chr19 | 49977465 | 49976715 | 49978215 | FLT3LG | 1 |
| chr10 | 97515672 | 97514922 | 97516422 | ENTPD1 | 1 | chr6 | 31550202 | 31549452 | 31550952 | LTB | −1 |
| chr19 | 55128628 | 55127878 | 55129378 | LILRB1 | 1 | chr1 | 36948879 | 36948129 | 36949629 | CSF3R | −1 |
| chr19 | 55141907 | 55141157 | 55142657 | LILRB1 | 1 | chr19 | 51728354 | 51727604 | 51729104 | CD33 | 1 |
| chr19 | 55141908 | 55141158 | 55142658 | LILRB1 | 1 | chr19 | 54850421 | 54849671 | 54851171 | LILRA4 | −1 |
| chr19 | 55141967 | 55141217 | 55142717 | LILRB1 | 1 | chr12 | 25205446 | 25204696 | 25206196 | LRMP | 1 |
| chr1 | 157670647 | 157669897 | 157671397 | FCRL3 | −1 | chr1 | 153348125 | 153347375 | 153348875 | S100A12 | −1 |
| chr19 | 51920952 | 51920202 | 51921702 | SIGLEC10 | −1 | chr2 | 218990727 | 218989977 | 218991477 | CXCR2 | 1 |
| chr2 | 208527099 | 208526349 | 208527849 | AC079767.4 | 1 | chr2 | 218990012 | 218989262 | 218990762 | CXCR2 | 1 |
| chr1 | 157746912 | 157746162 | 157747662 | FCRL2 | −1 | chr17 | 32646055 | 32645305 | 32646805 | CCL8 | 1 |
| chr21 | 15955723 | 15954973 | 15956473 | SAMSN1 | −1 | chr17 | 34391640 | 34390890 | 34392390 | CCL18 | 1 |
| chr21 | 15918681 | 15917931 | 15919431 | SAMSN1 | −1 | chr9 | 34691274 | 34690524 | 34692024 | CCL19 | −1 |
| chr1 | 214776538 | 214775788 | 214777288 | CENPF | 1 | chrX | 123480194 | 123479444 | 123480944 | SH2D1A | 1 |
| chr2 | 10262455 | 10261705 | 10263205 | RRM2 | 1 | chr1 | 92949511 | 92948761 | 92950261 | GFT1 | −1 |
| chr13 | 41240734 | 41239984 | 41241484 | FOXO1 | −1 | chr11 | 60869867 | 60869117 | 60870617 | CD5 | 1 |
| chr19 | 19302967 | 19302217 | 19303717 | MEF2B | −1 | chr16 | 21663981 | 21663231 | 21664731 | IGSF6 | −1 |
| chr3 | 154797436 | 154796686 | 154798186 | MME | 1 | chr5 | 131409483 | 131408733 | 131410233 | CSF2 | 1 |
| chr3 | 154797704 | 154796954 | 154798454 | MME | 1 | chr10 | 6104288 | 6103538 | 6105038 | IL2RA | −1 |
| chr3 | 154797952 | 154797202 | 154798702 | MME | 1 | chr1 | 117297007 | 117296257 | 117297757 | CD2 | 1 |
| chr3 | 154798078 | 154797328 | 154798828 | MME | 1 | chr5 | 131879214 | 131878464 | 131879964 | IL5 | −1 |
| chr1 | 153363664 | 153362914 | 153364414 | S100A8 | −1 | chr1 | 110453255 | 110452505 | 110454005 | CSF1 | 1 |
| chr1 | 160832550 | 160831800 | 160833300 | CD244 | −1 | chr1 | 1149512 | 1148762 | 1150262 | TNFRSF4 | −1 |
| chr1 | 3773845 | 3773095 | 3774595 | DFFB | 1 | chr2 | 85921414 | 85920664 | 85922164 | GNLY | 1 |
| chr16 | 3701838 | 3701088 | 3702588 | DNASE1 | 1 | chr20 | 24929866 | 24929116 | 24930616 | CST7 | 1 |
| chr3 | 58200398 | 58199648 | 58201148 | DNASE1L3 | −1 | chrX | 49121288 | 49120538 | 49122038 | FOXP3 | −1 |
| chr12 | 112563349 | 112562599 | 112564099 | TRAFD1 | 1 | chr1 | 169555826 | 169555076 | 169556576 | F5 | −1 |
| chr12 | 7060434 | 7059684 | 7061184 | PTPN6 | 1 | chr15 | 60919662 | 60918912 | 60920412 | RORA | −1 |
| chr12 | 7055739 | 7054989 | 7056489 | PTPN6 | 1 | chr19 | 41870078 | 41869328 | 41870828 | B9D2 | −1 |
| chr2 | 192015986 | 192015236 | 192016736 | STAT4 | −1 | chr19 | 55385736 | 55384986 | 55386486 | FCAR | 1 |
| chr2 | 192016322 | 192015572 | 192017072 | STAT4 | −1 | chr19 | 54824409 | 54823659 | 54825159 | LILRAS | −1 |
| chr9 | 5450525 | 5449775 | 5451275 | CD274 | 1 | chr2 | 25565459 | 25564709 | 25566209 | DNMT3A | −1 |
| chr8 | 39770803 | 39770053 | 39771553 | IDO1 | 1 | chr2 | 25475184 | 25474434 | 25475934 | DNMT3A | −1 |
| chr6 | 32821938 | 32821188 | 32822688 | PSMB9 | 1 | chr2 | 25564784 | 25564034 | 25565534 | DNMT3A | −1 |
| chr6 | 32821755 | 32821005 | 32822505 | TAP1 | −1 | chr2 | 216946539 | 216945789 | 216947289 | PECR | −1 |
| chr4 | 76957350 | 76956600 | 76958100 | CXCL11 | −1 | chr3 | 133118839 | 133118089 | 133119589 | BFSP2 | 1 |
| chr4 | 15779901 | 15779151 | 15780651 | CD38 | 1 | chr6 | 26235216 | 26234466 | 26235966 | HIST1H1D | −1 |
| chr12 | 10542617 | 10541867 | 10543367 | KLRK1 | −1 | chr6 | 26156559 | 26155809 | 26157309 | HIST1H1E | 1 |
| chr13 | 99913998 | 99913248 | 99914748 | GPR18 | −1 | chr6 | 26183958 | 26183208 | 26184708 | HIST1H2BE | 1 |
| chr16 | 11350036 | 11349286 | 11350786 | SOCS1 | −1 | chr6 | 26020718 | 26019968 | 26021468 | HIST1H3A | 1 |
| chr16 | 85932409 | 85931659 | 85933159 | IRF8 | 1 | chr6 | 26199521 | 26198771 | 26200271 | HIST1H3D | −1 |
| chr17 | 40264751 | 40264001 | 40265501 | DHX58 | −1 | chr6 | 7108041 | 7107291 | 7108791 | RREB1 | 1 |
| chr17 | 32597240 | 32596490 | 32597990 | CCL7 | 1 | chr6 | 144471663 | 144470913 | 144472413 | STX11 | 1 |
| chr19 | 52255146 | 52254396 | 52255896 | FPR1 | −1 | chr7 | 105517050 | 105516300 | 105517800 | ATXN7L1 | −1 |
| chr2 | 7017796 | 7017046 | 7018546 | RSAD2 | 1 | chr8 | 58907113 | 58906363 | 58907863 | FAM110B | 1 |
| chr2 | 152214106 | 152213356 | 152214856 | TNFAI6 | 1 | chr10 | 72362531 | 72361781 | 72363281 | PRF1 | −1 |
| chr20 | 56195529 | 56194779 | 56196279 | ZBP1 | −1 | chr1 | 198608170 | 198607420 | 198608920 | PTPRC | 1 |
| chr22 | 36044442 | 36043692 | 36045192 | APOL6 | 1 | chr1 | 155162706 | 155161956 | 155163456 | MUC1 | −1 |
| chr3 | 150996255 | 150995505 | 150997005 | P2RY14 | −1 | chr1 | 209979389 | 209978639 | 209980139 | IRF6 | −1 |
| chr3 | 121774221 | 121773471 | 121774971 | CD86 | 1 | chr1 | 153538341 | 153537591 | 153539091 | S100A2 | −1 |
| chr8 | 79717758 | 79717008 | 79718508 | IL7 | −1 | chr1 | 201252580 | 201251830 | 201253330 | PKP1 | 1 |
| chr20 | 44746911 | 44746161 | 44747661 | CD40 | 1 | chr1 | 114430169 | 114429419 | 114430919 | BCL2L15 | −1 |
| chr19 | 10381511 | 10380761 | 10382261 | ICAM1 | 1 | chr1 | 27189633 | 27188883 | 27190383 | SFN | 1 |
| chr14 | 20937538 | 20936788 | 20938288 | PNP | 1 | chr1 | 86889769 | 86889019 | 86890519 | CLCA2 | 1 |
| chr3 | 172241297 | 172240547 | 172242047 | TNFSF10 | −1 | chr1 | 153433177 | 153432427 | 153433927 | S100A7 | −1 |
| chr3 | 172241265 | 172240515 | 172242015 | TNFSF10 | −1 | chr10 | 81708859 | 81708109 | 81709609 | SFTPD | −1 |
| chr1 | 160709055 | 160708305 | 160709805 | SLAMF7 | 1 | chr10 | 5566924 | 5566174 | 5567674 | CALML3 | 1 |
| chr11 | 104905876 | 104905126 | 104906626 | CASP1 | −1 | chr11 | 1036706 | 1035956 | 1037456 | MUC6 | −1 |
| chr11 | 4414926 | 4414176 | 4415676 | TRIM21 | −1 | chr11 | 1244296 | 1243546 | 1245046 | MUC5B | 1 |
| chr12 | 53601091 | 53600341 | 53601841 | ITGB7 | −1 | chr11 | 120009161 | 120008411 | 120009911 | TRIM29 | −1 |
| chr2 | 85811531 | 85810781 | 85812281 | VAMP5 | 1 | chr11 | 71901258 | 71900508 | 71902008 | FOLR1 | 1 |
| chr6 | 32920899 | 32920149 | 32921649 | HLA-DMA | −1 | chr12 | 57634475 | 57633725 | 57635225 | NDUFA4L2 | −1 |
| chr16 | 27325234 | 27324484 | 27325984 | IL4R | 1 | chr12 | 52914471 | 52913721 | 52915221 | KRT5 | −1 |
| chr22 | 37546030 | 37545280 | 37546780 | IL2RB | −1 | chr12 | 6484390 | 6483640 | 6485140 | SCNN1A | −1 |
| chr21 | 34602279 | 34601529 | 34603029 | IFNAR2 | 1 | chr12 | 6484905 | 6484155 | 6485655 | SCNN1A | −1 |
| chr6 | 29909037 | 29908287 | 29909787 | HLA-A | 1 | chr12 | 6486523 | 6485773 | 6487273 | SCNN1A | −1 |
| chr6 | 33281989 | 33281239 | 33282739 | TAPBP | −1 | chr12 | 52887041 | 52886291 | 52887791 | KRT6A | −1 |
| chr15 | 45003675 | 45002925 | 45004425 | B2M | 1 | chr14 | 36989433 | 36988683 | 36990183 | NKX2-1 | −1 |
| chr1 | 89664615 | 89663865 | 89665365 | GBP4 | −1 | chr14 | 36988903 | 36988153 | 36989653 | NKX2-1 | −1 |
| chr1 | 36107445 | 36106695 | 36108195 | PSMB2 | −1 | chr15 | 41786073 | 41785323 | 41786823 | ITPKA | 1 |
| chr1 | 36097138 | 36096388 | 36097888 | PSMB2 | −1 | chr15 | 59903983 | 59903233 | 59904733 | GCNT3 | 1 |
| chr1 | 36107152 | 36106402 | 36107902 | PSMB2 | −1 | chr16 | 56716336 | 56715586 | 56717086 | MT1X | 1 |
| chr13 | 43566366 | 43565616 | 43567116 | EPSTI1 | −1 | chr16 | 5116111 | 5115361 | 5116861 | C16orf89 | −1 |
| chr14 | 100843142 | 100842392 | 100843892 | WARS | −1 | chr17 | 48133804 | 48133054 | 48134554 | ITGA3 | 1 |
| chr16 | 56642111 | 56641361 | 56642861 | MT2A | 1 | chr17 | 39769005 | 39768255 | 39769755 | KRT16 | −1 |
| chr12 | 108733094 | 108732344 | 108733844 | CMKLR1 | −1 | chr17 | 39661849 | 39661099 | 39662599 | KRT13 | −1 |
| chr12 | 108714439 | 108713689 | 108715189 | CMKLR1 | −1 | chr17 | 39678665 | 39677915 | 39679415 | KRT15 | −1 |
| chr16 | 67970990 | 67970240 | 67971740 | PSMB10 | −1 | chr18 | 61254600 | 61253850 | 61255350 | SERPINB13 | 1 |
| chr6 | 32557625 | 32556875 | 32558375 | HLA-DRB1 | −1 | chr18 | 29027758 | 29027008 | 29028508 | DSG3 | 1 |
| chr4 | 76928641 | 76927891 | 76929391 | CXCL9 | −1 | chr19 | 50869087 | 50868337 | 50869837 | NAPSA | −1 |
| chr6 | 32605134 | 32604384 | 32605884 | HLA-DQA1 | 1 | chr19 | 15083730 | 15082980 | 15084480 | SLC1A6 | −1 |
| chr17 | 34207512 | 34206762 | 34208262 | CCL5 | −1 | chr2 | 241375088 | 241374338 | 241375838 | GPC1 | 1 |
| chr12 | 9913497 | 9912747 | 9914247 | CD69 | −1 | chr2 | 234826043 | 234825293 | 234826793 | TRPM8 | 1 |
| chr6 | 391739 | 390989 | 392489 | IRF4 | 1 | chr22 | 37823505 | 37822755 | 37824255 | ELFN2 | −1 |
| chr14 | 24630262 | 24629512 | 24631012 | IRF9 | 1 | chr3 | 149688896 | 149688146 | 149689646 | PFN2 | −1 |
| chr17 | 40540586 | 40539836 | 40541336 | STAT3 | −1 | chr3 | 169587653 | 169586903 | 169588403 | LRRC31 | −1 |
| chr5 | 131826490 | 131825740 | 131827240 | IRF1 | −1 | chr3 | 189349216 | 189348466 | 189349966 | TP63 | 1 |
| chr10 | 6019553 | 6018803 | 6020303 | IL15RA | −1 | chr3 | 189507448 | 189506698 | 189508198 | TP63 | 1 |
| chr17 | 32582304 | 32581554 | 32583054 | CCL2 | 1 | chr4 | 15940363 | 15939613 | 15941113 | FGFBP1 | −1 |
| chr1 | 101185316 | 101184566 | 101186066 | VCAM1 | 1 | chr5 | 147258274 | 147257524 | 147259024 | SCGB3A2 | 1 |
| chr1 | 168545711 | 168544961 | 168546461 | XCL1 | 1 | chr6 | 131949601 | 131948851 | 131950351 | ENPP3 | 1 |
| chr4 | 103422486 | 103421736 | 103423236 | NFKB1 | 1 | chr7 | 5632454 | 5631704 | 5633204 | FSCN1 | 1 |
| chr14 | 62164340 | 62163590 | 62165090 | HIF1A | 1 | chr7 | 20257013 | 20256263 | 20257763 | MACC1 | −1 |
| chr14 | 62162118 | 62161368 | 62162868 | HIF1A | 1 | chr7 | 23286316 | 23285566 | 23287066 | GPNMB | 1 |
| chr1 | 156571262 | 156570512 | 156572012 | GPATCH4 | −1 | chr8 | 40010989 | 40010239 | 40011739 | C8orf4 | 1 |
| chr12 | 58146304 | 58145554 | 58147054 | CDK4 | −1 | chr8 | 72987852 | 72987102 | 72988602 | TRPA1 | −1 |
| chr19 | 18549111 | 18548361 | 18549861 | ISYNA1 | −1 | chr8 | 80680092 | 80679342 | 80680842 | HEY1 | −1 |
| chr19 | 18548119 | 18547369 | 18548869 | ISYNA1 | −1 | chr9 | 33447609 | 33446859 | 33448359 | AQP3 | −1 |
| chr19 | 18548972 | 18548222 | 18549722 | ISYNA1 | −1 | chrX | 150151759 | 150151009 | 150152509 | HMGB3 | 1 |
| chr19 | 47634120 | 47633370 | 47634870 | SAE1 | 1 | chrX | 152953554 | 152952804 | 152954304 | SLC6A8 | 1 |
| chr19 | 47634079 | 47633329 | 47634829 | SAE1 | 1 | chr18 | 28622707 | 28621957 | 28623457 | DSC3 | −1 |
| chr2 | 217362912 | 217362162 | 217363662 | RPL37A | 1 | chr2 | 46524541 | 46523791 | 46525291 | EPAS1 | 1 |
| chr1 | 150552066 | 150551316 | 150552816 | MCL1 | −1 | chr19 | 4229495 | 4228745 | 4230245 | EBI3 | 1 |
| chr1 | 79115516 | 79114766 | 79116266 | IFI44 | 1 | chr15 | 80263788 | 80263038 | 80264538 | BCL2A1 | −1 |
| chr1 | 948803 | 948053 | 949553 | ISG15 | 1 | chr1 | 32801980 | 32801230 | 32802730 | MARCKSL1 | −1 |
| chr10 | 91087651 | 91086901 | 91088401 | IFIT3 | 1 | chr11 | 33913836 | 33913086 | 33914586 | LMO2 | −1 |
| chr10 | 91152303 | 91151553 | 91153053 | IFIT1 | 1 | chr11 | 33891371 | 33890621 | 33892121 | LMO2 | −1 |
| chr12 | 113376157 | 113375407 | 113376907 | OAS3 | 1 | chr11 | 33891509 | 33890759 | 33892259 | LMO2 | −1 |
| chr12 | 121477045 | 121476295 | 121477795 | OASL | −1 | chr6 | 32498064 | 32497314 | 32498814 | HLA-DRB5 | −1 |
| chr12 | 113344588 | 113343838 | 113345338 | OAS1 | 1 | chrHSC | 32430644 | 32429894 | 32431394 | HLA-DRB3 | −1 |
| chr12 | 85306655 | 85305905 | 85307405 | SLC6A15 | −1 | chrHSC | 32524536 | 32523786 | 32525286 | HLA-DRB4 | −1 |
| chr13 | 37006495 | 37005745 | 37007245 | CCNA1 | 1 | chr12 | 111126514 | 111125764 | 111127264 | HVCN1 | −1 |
| chr14 | 88459615 | 88458865 | 88460365 | GALC | −1 | chr15 | 57592563 | 57591813 | 57593313 | LINC00926 | 1 |
| chr14 | 88460009 | 88459259 | 88460759 | GALC | −1 | chr15 | 57596923 | 57596173 | 57597673 | LINC00926 | 1 |
| chr14 | 94577161 | 94576411 | 94577911 | IFI27 | 1 | chr4 | 165305202 | 165304452 | 165305952 | 1-Mar | −1 |
| chr15 | 39873294 | 39872544 | 39874044 | THBS1 | 1 | chr5 | 57787262 | 57786512 | 57788012 | GAPT | 1 |
| chr17 | 41158757 | 41158007 | 41159507 | IFI35 | 1 | chr5 | 140013286 | 140012536 | 140014036 | CD14 | −1 |
| chr17 | 40701232 | 40700482 | 40701982 | HSD17B1 | 1 | chr5 | 140013035 | 140012285 | 140013785 | CD14 | −1 |
| chr19 | 17516457 | 17515707 | 17517207 | BST2 | −1 | chr5 | 82767284 | 82766534 | 82768034 | VCAN | 1 |
| chr21 | 42733870 | 42733120 | 42734620 | MX2 | 1 | chr12 | 7902069 | 7901319 | 7902819 | CLEC4C | −1 |
| chr22 | 33197687 | 33196937 | 33198437 | TIMP3 | 1 | chr17 | 1665253 | 1664503 | 1666003 | SERPINF1 | 1 |
| chr22 | 18632666 | 18631916 | 18633416 | USP18 | 1 | chr20 | 23030378 | 23029628 | 23031128 | THBD | −1 |
| chr3 | 79816965 | 79816215 | 79817715 | ROBO1 | −1 | chrX | 1455509 | 1454759 | 1456259 | IL3RA | 1 |
| chr4 | 74735121 | 74734371 | 74735871 | CXCL1 | 1 | chrY | 1405508 | 1404758 | 1406258 | IL3RA | 1 |
| chr4 | 169239958 | 169239208 | 169240708 | DDX60 | −1 | chr3 | 111852101 | 111851351 | 111852851 | GCSAM | −1 |
| chr4 | 89300015 | 89299265 | 89300765 | HERC6 | 1 | chr8 | 119294481 | 119293731 | 119295231 | AC023590.1 | 1 |
| chr6 | 29794756 | 29794006 | 29795506 | HLA-G | 1 | chr9 | 95820989 | 95820239 | 95821739 | SUSD3 | 1 |
| chr8 | 86376081 | 86375331 | 86376831 | CA2 | 1 | chr18 | 61554939 | 61554189 | 61555689 | SERPINB2 | 1 |
| chr8 | 144099945 | 144099195 | 144100695 | LY6E | 1 | chr3 | 71834212 | 71833462 | 71834962 | PROK2 | −1 |
| chr9 | 100881480 | 100880730 | 100882230 | TRIM14 | −1 | chr1 | 231560790 | 231560040 | 231561540 | EGLN1 | −1 |
| chr4 | 76944650 | 76943900 | 76945400 | CXCL10 | −1 | chr11 | 65084691 | 65083941 | 65085441 | CDC42EP2 | 1 |
| chr1 | 27998689 | 27997939 | 27999439 | IFI6 | −1 | chr11 | 118095809 | 118095059 | 118096559 | AMICA1 | −1 |
| chr1 | 79086088 | 79085338 | 79086838 | IFI44L | 1 | chr11 | 118084074 | 118083324 | 118084824 | AMICA1 | −1 |
| chr3 | 182880699 | 182879949 | 182881449 | LAMP3 | −1 | chr14 | 21538429 | 21537679 | 21539179 | ARHGEF40 | 1 |
| chr3 | 146262651 | 146261901 | 146263401 | PLSCR1 | −1 | chr16 | 12897874 | 12897124 | 12898624 | CPPED1 | −1 |
| chr11 | 313853 | 313103 | 314603 | IFITM1 | 1 | chr2 | 24714801 | 24714051 | 24715551 | NCOA1 | 1 |
| chr17 | 76976191 | 76975441 | 76976941 | LGALS3BP | −1 | chr20 | 54987314 | 54986564 | 54988064 | CASS4 | 1 |
| chr6 | 31324964 | 31324214 | 31325714 | HLA-B | −1 | chr20 | 54987167 | 54986417 | 54987917 | CASS4 | 1 |
| chr21 | 42792442 | 42791692 | 42793192 | MX1 | 1 | chr1 | 172628158 | 172627408 | 172628908 | FASLG | 1 |
| chr2 | 191878976 | 191878226 | 191879726 | STAT1 | −1 | chr19 | 2256416 | 2255666 | 2257166 | JSRP1 | −1 |
| chr11 | 615728 | 614978 | 616478 | IRF7 | −1 | chr2 | 89340242 | 89339492 | 89340992 | IGKV1-12 | −1 |
| chr11 | 615999 | 615249 | 616749 | IRF7 | −1 | chr22 | 24181193 | 24180443 | 24181943 | DERL3 | −1 |
| chr2 | 87089038 | 87088288 | 87089788 | CD8B | −1 | chr22 | 23229960 | 23229210 | 23230710 | IGLL5 | 1 |
| chrX | 21958691 | 21957941 | 21959441 | SMS | 1 | chr22 | 23222886 | 23222136 | 23223636 | IGLV3-1 | 1 |
| chr1 | 29213603 | 29212853 | 29214353 | EPB41 | 1 | chr22 | 22550113 | 22549363 | 22550863 | IGLV6-57 | 1 |
| chr1 | 29241087 | 29240337 | 29241837 | EPB41 | 1 | chr22 | 23264766 | 23264016 | 23265516 | IGLC7 | 1 |
| chr1 | 178694300 | 178693550 | 178695050 | RALGPS2 | 1 | chr13 | 99959659 | 99958909 | 99960409 | GPR183 | −1 |
| chr1 | 52870131 | 52869381 | 52870881 | ORC1 | −1 | chr19 | 16435628 | 16434878 | 16436378 | KLF2 | 1 |
| chr1 | 112282046 | 112281296 | 112282796 | FAM212B | −1 | chr1 | 157567870 | 157567120 | 157568620 | FCRL4 | −1 |
| chr1 | 112298419 | 112297669 | 112299169 | FAM212B | −1 | chr10 | 17270258 | 17269508 | 17271008 | VIM | 1 |
| chr1 | 157108159 | 157107409 | 157108909 | ETV3 | −1 | chr16 | 31366455 | 31365705 | 31367205 | ITGAX | 1 |
| chr1 | 154928580 | 154927830 | 154929330 | PBXIP1 | −1 | chr4 | 84035868 | 84035118 | 84036618 | PLAC8 | −1 |
| chr1 | 161632951 | 161632201 | 161633701 | FCGR2B | 1 | chr17 | 16875402 | 16874652 | 16876152 | TNFRSF13B | −1 |
| chr1 | 157963400 | 157962650 | 157964150 | KIRREL | 1 | chr1 | 145438469 | 145437719 | 145439219 | TXNIP | 1 |
| chr1 | 24829387 | 24828637 | 24830137 | RCAN3 | 1 | chr1 | 207095343 | 207094593 | 207096093 | FAIM3 | −1 |
| chr1 | 24828840 | 24828090 | 24829590 | RCAN3 | 1 | chr19 | 7767032 | 7766282 | 7767782 | FCER2 | −1 |
| chr1 | 24834083 | 24833333 | 24834833 | RCAN3 | 1 | chr19 | 7764365 | 7763615 | 7765115 | FCER2 | −1 |
| chr1 | 24840803 | 24840053 | 24841553 | RCAN3 | 1 | chr1 | 40563375 | 40562625 | 40564125 | PPT1 | −1 |
| chr1 | 36590821 | 36590071 | 36591571 | COL8A2 | −1 | chr1 | 1141951 | 1141201 | 1142701 | TNFRSF18 | −1 |
| chr1 | 156830686 | 156829936 | 156831436 | NTRK1 | 1 | chr11 | 67211263 | 67210513 | 67212013 | CORO1B | −1 |
| chr1 | 247581351 | 247580601 | 247582101 | NLRP3 | 1 | chr12 | 56546040 | 56545290 | 56546790 | MYL6B | 1 |
| chr1 | 247579457 | 247578707 | 247580207 | NLRP3 | 1 | chr15 | 85201794 | 85201044 | 85202544 | NMB | −1 |
| chr1 | 150240600 | 150239850 | 150241350 | C1orf54 | 1 | chr15 | 49338760 | 49338010 | 49339510 | SECISBP2L | −1 |
| chr1 | 111217655 | 111216905 | 111218405 | KCNA3 | −1 | chr16 | 30483979 | 30483229 | 30484729 | ITGAL | 1 |
| chr1 | 207627575 | 207626825 | 207628325 | CR2 | 1 | chr16 | 79634611 | 79633861 | 79635361 | MAF | −1 |
| chr1 | 158801107 | 158800357 | 158801857 | MNDA | 1 | chr17 | 57409063 | 57408313 | 57409813 | YPEL2 | 1 |
| chr1 | 17634692 | 17633942 | 17635442 | PADI4 | 1 | chr17 | 6917814 | 6917064 | 6918564 | C17orf49 | 1 |
| chr1 | 183559716 | 183558966 | 183560466 | NCF2 | −1 | chr2 | 3523507 | 3522757 | 3524257 | ADI1 | −1 |
| chr1 | 111743393 | 111742643 | 111744143 | CHI312 | 1 | chr2 | 30454397 | 30453647 | 30455147 | LBH | 1 |
| chr1 | 67773047 | 67772297 | 67773797 | IL12RB2 | 1 | chr2 | 38893052 | 38892302 | 38893802 | GALM | 1 |
| chr1 | 20915441 | 20914691 | 20916191 | CDA | 1 | chr2 | 109271493 | 109270743 | 109272243 | LIMS1 | 1 |
| chr1 | 182761394 | 182760644 | 182762144 | NPL | 1 | chr2 | 95691429 | 95690679 | 95692179 | MAL | 1 |
| chr1 | 158149737 | 158148987 | 158150487 | CD1D | 1 | chr3 | 191046874 | 191046124 | 191047624 | CCDC50 | 1 |
| chr1 | 160617085 | 160616335 | 160617835 | SLAMF1 | −1 | chr5 | 138862375 | 138861625 | 138863125 | TMEM173 | −1 |
| chr1 | 161494036 | 161493286 | 161494786 | HSPA6 | 1 | chr6 | 36355560 | 36354810 | 36356310 | ETV7 | −1 |
| chr1 | 2398898 | 2398148 | 2399648 | PLCH2 | 1 | chr6 | 36355577 | 36354827 | 36356327 | ETV7 | −1 |
| chr1 | 49242590 | 49241840 | 49243340 | BEND5 | −1 | chr6 | 135818883 | 135818133 | 135819633 | AHI1 | −1 |
| chr1 | 158223927 | 158223177 | 158224677 | CD1A | 1 | chr7 | 37488852 | 37488102 | 37489602 | ELMO1 | −1 |
| chr1 | 160765948 | 160765198 | 160766698 | LY9 | 1 | chr8 | 29208185 | 29207435 | 29208935 | DUSP4 | −1 |
| chr1 | 39549034 | 39548284 | 39549784 | MACF1 | 1 | chr8 | 134072603 | 134071853 | 134073353 | SLA | −1 |
| chr1 | 18957500 | 18956750 | 18958250 | PAX7 | 1 | chr8 | 134115310 | 134114560 | 134116060 | SLA | −1 |
| chr1 | 159259504 | 159258754 | 159260254 | FCER1A | 1 | chr12 | 6898638 | 6897888 | 6899388 | CD4 | 1 |
| chr1 | 204135450 | 204134700 | 204136200 | REN | −1 | chr12 | 47473386 | 47472636 | 47474136 | PCED1B | 1 |
| chr1 | 159046691 | 159045941 | 159047441 | AIM2 | −1 | chr14 | 45722605 | 45721855 | 45723355 | MIS18BP1 | −1 |
| chr1 | 115238207 | 115237457 | 115238957 | AMPD1 | −1 | chr16 | 57662419 | 57661669 | 57663169 | GPR56 | 1 |
| chr1 | 55107466 | 55106716 | 55108216 | MROH7 | 1 | chr16 | 57653909 | 57653159 | 57654659 | GPR56 | 1 |
| chr1 | 32716840 | 32716090 | 32717590 | LCK | 1 | chr16 | 57673206 | 57672456 | 57673956 | GPR56 | 1 |
| chr1 | 32739711 | 32738961 | 32740461 | LCK | 1 | chr19 | 36233348 | 36232598 | 36234098 | IGFLR1 | −1 |
| chr1 | 28261504 | 28260754 | 28262254 | SMPDL3B | 1 | chr22 | 35776828 | 35776078 | 35777578 | HMOX1 | 1 |
| chr1 | 87099958 | 87099208 | 87100708 | CLCA3P | 1 | chr19 | 11689794 | 11689044 | 11690544 | ACP5 | −1 |
| chr10 | 45455240 | 45454490 | 45455990 | RASSF4 | 1 | chr19 | 11688553 | 11687803 | 11689303 | ACP5 | −1 |
| chr10 | 45869675 | 45868925 | 45870425 | ALOX5 | 1 | chr19 | 11689592 | 11688842 | 11690342 | ACP5 | −1 |
| chr10 | 85954414 | 85953664 | 85955164 | CDHR1 | 1 | chr1 | 230202956 | 230202206 | 230203706 | GALNT2 | 1 |
| chr10 | 125851970 | 125851220 | 125852720 | CHST15 | −1 | chr1 | 113249749 | 113248999 | 113250499 | RHOC | −1 |
| chr10 | 99161100 | 99160350 | 99161850 | RRP12 | −1 | chr1 | 150122170 | 150121420 | 150122920 | PLEKHO1 | 1 |
| chr10 | 73848531 | 73847781 | 73849281 | SPOCK2 | −1 | chr10 | 17686124 | 17685374 | 17686874 | STAM | 1 |
| chr10 | 73848790 | 73848040 | 73849540 | SPOCK2 | −1 | chr10 | 90751147 | 90750397 | 90751897 | ACTA2 | −1 |
| chr10 | 94447945 | 94447195 | 94448695 | HHEX | 1 | chr10 | 90712580 | 90711830 | 90713330 | ACTA2 | −1 |
| chr10 | 102279591 | 102278841 | 102280341 | SEC31B | −1 | chr10 | 124134212 | 124133462 | 124134962 | PLEKHA1 | 1 |
| chr10 | 14372883 | 14372133 | 14373633 | FRMD4A | −1 | chr10 | 8096656 | 8095906 | 8097406 | GATA3 | 1 |
| chr10 | 49812997 | 49812247 | 49813747 | ARHGAP22 | −1 | chr10 | 105212075 | 105211325 | 105212825 | CALHM2 | −1 |
| chr10 | 54531460 | 54530710 | 54532210 | MBL2 | −1 | chr10 | 124221041 | 124220291 | 124221791 | HTRA1 | 1 |
| chr10 | 95372345 | 95371595 | 95373095 | PDE6C | 1 | chr11 | 18548779 | 18548029 | 18549529 | TSG101 | −1 |
| chr11 | 64570713 | 64569963 | 64571463 | MAP4K2 | −1 | chr11 | 59578345 | 59577595 | 59579095 | MRPL16 | −1 |
| chr11 | 65154070 | 65153320 | 65154820 | FRMD8 | 1 | chr11 | 3078667 | 3077917 | 3079417 | CARS | −1 |
| chr11 | 75062749 | 75061999 | 75063499 | ARRB1 | −1 | chr12 | 6875543 | 6874793 | 6876293 | PTMS | 1 |
| chr11 | 66512207 | 66511457 | 66512957 | C11orf80 | 1 | chr12 | 102224716 | 102223966 | 102225466 | GNPTAB | −1 |
| chr11 | 18034709 | 18033959 | 18035459 | SERGEF | −1 | chr12 | 57873633 | 57872883 | 57874383 | ARHGAP9 | −1 |
| chr11 | 289138 | 288388 | 289888 | ATHL1 | 1 | chr12 | 57871728 | 57870978 | 57872478 | ARHGAP9 | −1 |
| chr11 | 64512928 | 64512178 | 64513678 | RASGRP2 | −1 | chr12 | 57873424 | 57872674 | 57874174 | ARHGAP9 | −1 |
| chr11 | 64511630 | 64510880 | 64512380 | RASGRP2 | −1 | chr12 | 55378456 | 55377706 | 55379206 | TESPA1 | −1 |
| chr11 | 64512329 | 64511579 | 64513079 | RASGRP2 | −1 | chr12 | 6451280 | 6450530 | 6452030 | TNFRSF1A | −1 |
| chr11 | 36397555 | 36396805 | 36398305 | PRR5L | 1 | chr12 | 45270880 | 45270130 | 45271630 | NELL2 | −1 |
| chr11 | 72929344 | 72928594 | 72930094 | P2RY2 | 1 | chr13 | 80055287 | 80054537 | 80056037 | NDFIP2 | 1 |
| chr11 | 72929501 | 72928751 | 72930251 | P2RY2 | 1 | chr13 | 31309645 | 31308895 | 31310395 | ALOX5AP | 1 |
| chr11 | 60719257 | 60718507 | 60720007 | SLC15A3 | −1 | chr13 | 31287614 | 31286864 | 31288364 | ALOX5AP | 1 |
| chr11 | 102188226 | 102187476 | 102188976 | BIRC3 | 1 | chr14 | 91720269 | 91719519 | 91721019 | GPR68 | −1 |
| chr11 | 88910620 | 88909870 | 88911370 | TYR | 1 | chr14 | 94443137 | 94442387 | 94443887 | ASB2 | −1 |
| chr11 | 59950696 | 59949946 | 59951446 | MS4A6A | −1 | chr15 | 34875771 | 34875021 | 34876521 | GOLGA8B | −1 |
| chr11 | 129245835 | 129245085 | 129246585 | BARX2 | 1 | chr15 | 34828222 | 34827472 | 34828972 | GOLGA8B | −1 |
| chr11 | 104893895 | 104893145 | 104894645 | CASP5 | −1 | chr16 | 30366682 | 30365932 | 30367432 | CD2BP2 | −1 |
| chr11 | 118213459 | 118212709 | 118214209 | CD3D | −1 | chr16 | 30366255 | 30365505 | 30367005 | CD2BP2 | −1 |
| chr11 | 59856137 | 59855387 | 59856887 | MS4A2 | 1 | chr16 | 27280115 | 27279365 | 27280865 | NSMCE1 | −1 |
| chr11 | 60739152 | 60738402 | 60739902 | CD6 | 1 | chr17 | 62207485 | 62206735 | 62208235 | ERN1 | −1 |
| chr11 | 118215071 | 118214321 | 118215821 | CD3G | 1 | chr17 | 73512606 | 73511856 | 73513356 | TSEN54 | 1 |
| chr11 | 57158130 | 57157380 | 57158880 | PRG2 | −1 | chr17 | 47287589 | 47286839 | 47288339 | ABI3 | 1 |
| chr11 | 118175260 | 118174510 | 118176010 | CD3E | 1 | chr17 | 33640683 | 33639933 | 33641433 | RP11-47L3.1 | 1 |
| chr11 | 64826021 | 64825271 | 64826771 | NAALADL1 | −1 | chr17 | 33640521 | 33639771 | 33641271 | RP11-47L3.1 | 1 |
| chr11 | 59824156 | 59823406 | 59824906 | MS4A3 | 1 | chr2 | 242212301 | 242211551 | 242213051 | HDLBP | −1 |
| chr11 | 1432382 | 1431632 | 1433132 | BRSK2 | 1 | chr2 | 85637676 | 85636926 | 85638426 | CAPG | −1 |
| chr11 | 1411128 | 1410378 | 1411878 | BRSK2 | 1 | chr20 | 35274619 | 35273869 | 35275369 | SLA2 | −1 |
| chr11 | 1431672 | 1430922 | 1432422 | BRSK2 | 1 | chr22 | 35796056 | 35795306 | 35796806 | MCM5 | 1 |
| chr11 | 102745764 | 102745014 | 102746514 | MMP12 | −1 | chr22 | 37415702 | 37414952 | 37416452 | MPST | 1 |
| chr12 | 27677139 | 27676389 | 27677889 | PPFIBP1 | 1 | chr22 | 38035482 | 38034732 | 38036232 | SH3BP1 | 1 |
| chr12 | 31744031 | 31743281 | 31744781 | DENND5B | −1 | chr22 | 37257030 | 37256280 | 37257780 | NCF4 | 1 |
| chr12 | 96390143 | 96389393 | 96390893 | HAL | −1 | chr3 | 160117092 | 160116342 | 160117842 | SMC4 | 1 |
| chr12 | 10282836 | 10282086 | 10283586 | CLEC7A | −1 | chr3 | 52280225 | 52279475 | 52280975 | PPM1M | 1 |
| chr12 | 22487648 | 22486898 | 22488398 | ST8SIA1 | −1 | chr4 | 47487305 | 47486555 | 47488055 | ATP10D | 1 |
| chr12 | 54689542 | 54688792 | 54690292 | NFE2 | −1 | chr4 | 170533780 | 170533030 | 170534530 | NEK1 | −1 |
| chr12 | 8219067 | 8218317 | 8219817 | C3AR1 | −1 | chr4 | 47465736 | 47464986 | 47466486 | COMMD8 | −1 |
| chr12 | 123466196 | 123465446 | 123466946 | ABCB9 | −1 | chr5 | 65892189 | 65891439 | 65892939 | MAST4 | 1 |
| chr12 | 58161034 | 58160284 | 58161784 | CYP27B1 | −1 | chr5 | 54830797 | 54830047 | 54831547 | PPAP2A | −1 |
| chr12 | 56324756 | 56324006 | 56325506 | DGKA | 1 | chr5 | 93954052 | 93953302 | 93954802 | ANKRD32 | 1 |
| chr12 | 10573149 | 10572399 | 10573899 | KLRC3 | −1 | chr6 | 33043703 | 33042953 | 33044453 | HLA-DPB1 | 1 |
| chr12 | 10562356 | 10561606 | 10563106 | KLRC4 | −1 | chr6 | 33048487 | 33047737 | 33049237 | HLA-DPA1 | −1 |
| chr12 | 68619601 | 68618851 | 68620351 | IL26 | −1 | chr6 | 159082351 | 159081601 | 159083101 | SYTL3 | 1 |
| chr12 | 10460417 | 10459667 | 10461167 | KLRD1 | 1 | chr7 | 45018697 | 45017947 | 45019447 | MYO1G | −1 |
| chr12 | 10457049 | 10456299 | 10457799 | KLRD1 | 1 | chr7 | 110731062 | 110730312 | 110731812 | LRRN3 | 1 |
| chr12 | 122457328 | 122456578 | 122458078 | BCL7A | 1 | chr9 | 139743546 | 139742796 | 139744296 | PHPT1 | 1 |
| chr12 | 12837455 | 12836705 | 12838205 | GPR19 | −1 | chr9 | 139891255 | 139890505 | 139892005 | CLIC3 | −1 |
| chr12 | 9980077 | 9979327 | 9980827 | KLRF1 | 1 | chrX | 128913960 | 128913210 | 128914710 | SASH3 | 1 |
| chr12 | 8276228 | 8275478 | 8276978 | CLEC4A | 1 | chr1 | 200842083 | 200841333 | 200842833 | GPR25 | 1 |
| chr12 | 9822309 | 9821559 | 9823059 | CLEC2D | 1 | chr11 | 65647280 | 65646530 | 65648030 | CTSW | 1 |
| chr12 | 102591623 | 102590873 | 102592373 | PMCH | −1 | chr11 | 67205538 | 67204788 | 67206288 | PTPRCAP | −1 |
| chr12 | 1738363 | 1737613 | 1739113 | WNT5B | 1 | chr15 | 64332466 | 64331716 | 64333216 | DAPK2 | −1 |
| chr12 | 9760482 | 9759732 | 9761232 | KLRB1 | −1 | chr19 | 30155963 | 30155213 | 30156713 | PLEKHF1 | 1 |
| chr13 | 114898086 | 114897336 | 114898836 | RASA3 | −1 | chr19 | 6591163 | 6590413 | 6591913 | CD70 | −1 |
| chr13 | 72441330 | 72440580 | 72442080 | DACH1 | −1 | chr2 | 231090446 | 231089696 | 231091196 | SP140 | 1 |
| chr13 | 46961635 | 46960885 | 46962385 | KIAA0226L | −1 | chr19 | 55315095 | 55314345 | 55315845 | KIR2DL4 | 1 |
| chr13 | 70681344 | 70680594 | 70682094 | ATXN8OS | 1 | chr12 | 6308881 | 6308131 | 6309631 | CD9 | 1 |
| chr14 | 20881580 | 20880830 | 20882330 | TEP1 | −1 | chr11 | 111411384 | 111410634 | 111412134 | LAYN | 1 |
| chr14 | 76044960 | 76044210 | 76045710 | FLVCR2 | 1 | chr4 | 71532377 | 71531627 | 71533127 | IGJ | −1 |
| chr14 | 65171154 | 65170404 | 65171904 | PLEKHG3 | 1 | chr5 | 138725603 | 138724853 | 138726353 | MZB1 | −1 |
| chr14 | 52781113 | 52780363 | 52781863 | PTGER2 | 1 | chr22 | 29196585 | 29195835 | 29197335 | XBP1 | −1 |
| chr14 | 88471468 | 88470718 | 88472218 | GPR65 | 1 | chr19 | 8373239 | 8372489 | 8373989 | CD320 | −1 |
| chr14 | 99737565 | 99736815 | 99738315 | BCL11B | −1 | chr3 | 9811661 | 9810911 | 9812411 | CAMK1 | −1 |
| chr14 | 52734431 | 52733681 | 52735181 | PTGDR | 1 | chr10 | 11059893 | 11059143 | 11060643 | CELF2 | 1 |
| chr14 | 21249210 | 21248460 | 21249960 | RNASE6 | 1 | chr10 | 11047258 | 11046508 | 11048008 | CELF2 | 1 |
| chr14 | 81421387 | 81420637 | 81422137 | TSHR | 1 | chr10 | 11206992 | 11206242 | 11207742 | CELF2 | 1 |
| chr14 | 25519503 | 25518753 | 25520253 | STXBP6 | −1 | chr11 | 308163 | 307413 | 308913 | IFITM2 | 1 |
| chr14 | 24977471 | 24976721 | 24978221 | CMA1 | −1 | chr14 | 24616430 | 24615680 | 24617180 | PSME2 | −1 |
| chr14 | 25045466 | 25044716 | 25046216 | CTSG | −1 | chr21 | 26934456 | 26933706 | 26935206 | MIR155HG | 1 |
| chr14 | 21423611 | 21422861 | 21424361 | RNASE2 | 1 | chr21 | 26946292 | 26945542 | 26947042 | MIR155HG | 1 |
| chr14 | 47121028 | 47120278 | 47121778 | RPL10L | −1 | chr21 | 26934221 | 26933471 | 26934971 | MIR155HG | 1 |
| chr14 | 22356037 | 22355287 | 22356787 | TRAV12-2 | 1 | chr22 | 44395091 | 44394341 | 44395841 | PARVB | 1 |
| chr14 | 22337020 | 22336270 | 22337770 | TRAV13-1 | 1 | chr22 | 44420156 | 44419406 | 44420906 | PARVB | 1 |
| chr14 | 22386363 | 22385613 | 22387113 | TRAV13-2 | 1 | chr22 | 44422183 | 44421433 | 44422933 | PARVB | 1 |
| chr14 | 22520773 | 22520023 | 22521523 | TRAV21 | 1 | chr22 | 44464996 | 44464246 | 44465746 | PARVB | 1 |
| chr14 | 22446699 | 22445949 | 22447449 | TRAV8-6 | 1 | chr3 | 5020801 | 5020051 | 5021551 | BHLHE40 | 1 |
| chr14 | 22931924 | 22931174 | 22932674 | TRDC | 1 | chr12 | 4382938 | 4382188 | 4383688 | CCND2 | 1 |
| chr15 | 91427642 | 91426892 | 91428392 | FES | 1 | chr4 | 57547872 | 57547122 | 57548622 | HOPX | −1 |
| chri5 | 50411419 | 50410669 | 50412169 | ATP8B4 | −1 | chr4 | 57522688 | 57521938 | 57523438 | HOPX | −1 |
| chr15 | 58430395 | 58429645 | 58431145 | AQP9 | 1 | chr5 | 180467225 | 180466475 | 180467975 | BTNL9 | 1 |
| chr15 | 50558223 | 50557473 | 50558973 | HDC | −1 | chr2 | 183731890 | 183731140 | 183732640 | FRZB | −1 |
| chr15 | 93199188 | 93198438 | 93199938 | FAM174B | −1 | chr4 | 74486348 | 74485598 | 74487098 | RASSF6 | −1 |
| chr15 | 48498499 | 48497749 | 48499249 | SLC12A1 | 1 | chr4 | 74486287 | 74485537 | 74487037 | RASSF6 | −1 |
| chr15 | 43559055 | 43558305 | 43559805 | TGM5 | −1 | chr12 | 2986152 | 2985402 | 2986902 | FOXM1 | −1 |
| chr15 | 69606742 | 69605992 | 69607492 | PAQR5 | 1 | chr12 | 56123491 | 56122741 | 56124241 | CD63 | −1 |
| chr15 | 69591293 | 69590543 | 69592043 | PAQR5 | 1 | chr8 | 11725738 | 11724988 | 11726488 | CTSB | −1 |
| chr15 | 77517746 | 77516996 | 77518496 | LINC00597 | −1 | chr13 | 49345264 | 49344514 | 49346014 | PSME2P2 | 1 |
| chr16 | 67193834 | 67193084 | 67194584 | FBXL8 | 1 | chr13 | 49345759 | 49345009 | 49346509 | PSME2P2 | 1 |
| chr16 | 68034489 | 68033739 | 68035239 | DPEP2 | −1 | chr5 | 97549106 | 97548356 | 97549856 | R11-274E7.2 | 1 |
| chr16 | 3096682 | 3095932 | 3097432 | MMP25 | 1 | chr5 | 155885517 | 155884767 | 155886267 | CTB-56J15.1 | −1 |
| chr16 | 68482591 | 68481841 | 68483341 | SMPD3 | −1 | chr6 | 74161999 | 74161249 | 74162749 | MB21D1 | −1 |
| chr16 | 28996147 | 28995397 | 28996897 | LAT | 1 | chr2 | 87035519 | 87034769 | 87036269 | CD8A | −1 |
| chr16 | 28996386 | 28995636 | 28997136 | LAT | 1 | chr2 | 87018837 | 87018087 | 87019587 | CD8A | −1 |
| chr16 | 2581420 | 2580670 | 2582170 | CEMP1 | −1 | chr9 | 5510570 | 5509820 | 5511320 | PDCD1LG2 | 1 |
| chr16 | 50731050 | 50730300 | 50731800 | NOD2 | 1 | chr6 | 30457244 | 30456494 | 30457994 | HLA-E | 1 |
| chr16 | 3306627 | 3305877 | 3307377 | MEFV | −1 | chr12 | 6554033 | 6553283 | 6554783 | CD27 | 1 |
| chr16 | 74411776 | 74411026 | 74412526 | NPIPB15 | 1 | chr3 | 45986511 | 45985761 | 45987261 | CXCR6 | 1 |
| chr16 | 57438679 | 57437929 | 57439429 | CCL17 | 1 | chr14 | 24836117 | 24835367 | 24836867 | NFATC4 | 1 |
| chr16 | 57702099 | 57701349 | 57702849 | GPR97 | 1 | chr16 | 68119561 | 68118811 | 68120311 | NFATC3 | 1 |
| chr16 | 2521500 | 2520750 | 2522250 | NTN3 | 1 | chr18 | 60987361 | 60986611 | 60988111 | BCL2 | −1 |
| chr16 | 57392684 | 57391934 | 57393434 | CCL22 | 1 | chr19 | 18392432 | 18391682 | 18393182 | JUND | −1 |
| chr17 | 27071002 | 27070252 | 27071752 | TRAF4 | 1 | chr20 | 50159258 | 50158508 | 50160008 | NFATC2 | −1 |
| chr17 | 3819794 | 3819044 | 3820544 | P2RX1 | −1 | chr20 | 50179370 | 50178620 | 50180120 | NFATC2 | −1 |
| chr17 | 42634925 | 42634175 | 42635675 | FZD2 | 1 | chr1 | 59249785 | 59249035 | 59250535 | JUN | −1 |
| chr17 | 46507552 | 46506802 | 46508302 | SKAP1 | −1 | chr3 | 187463515 | 187462765 | 187464265 | BCL6 | −1 |
| chr17 | 3599698 | 3598948 | 3600448 | P2RX5 | −1 | chr3 | 187452695 | 187451945 | 187453445 | BCL6 | −1 |
| chr17 | 7482785 | 7482035 | 7483535 | CD68 | 1 | chr3 | 187454285 | 187453535 | 187455035 | BCL6 | −1 |
| chr17 | 40833861 | 40833111 | 40834611 | CCR10 | −1 | chr2 | 242801060 | 242800310 | 242801810 | PDCD1 | −1 |
| chr17 | 19140654 | 19139904 | 19141404 | EPN2 | 1 | chr3 | 27763989 | 27763239 | 27764739 | EOMES | −1 |
| chr17 | 15603091 | 15602341 | 15603841 | ZNF286A | 1 | chr12 | 68553527 | 68552777 | 68554277 | IFNG | −1 |
| chr17 | 1959604 | 1958854 | 1960354 | HIC1 | 1 | chr10 | 129924649 | 129923899 | 129925399 | MKI67 | −1 |
| chr17 | 1958392 | 1957642 | 1959142 | HIC1 | 1 | chr17 | 45810610 | 45809860 | 45811360 | TBX21 | 1 |
| chr17 | 7239848 | 7239098 | 7240598 | ACAP1 | 1 | chr8 | 60031767 | 60031017 | 60032517 | TOX | −1 |
| chr17 | 72462555 | 72461805 | 72463305 | CD300A | 1 | chr2 | 8818975 | 8818225 | 8819725 | ID2 | 1 |
| chr17 | 4545589 | 4544839 | 4546339 | ALOX15 | −1 | chr6 | 106534195 | 106533445 | 106534945 | PRDM1 | 1 |
| chr17 | 34345005 | 34344255 | 34345755 | CCL23 | −1 | chr6 | 106546736 | 106545986 | 106547486 | PRDM1 | 1 |
| chr17 | 7082883 | 7082133 | 7083633 | ASGR1 | −1 | chr14 | 75745477 | 75744727 | 75746227 | FOS | 1 |
| chr17 | 7018128 | 7017378 | 7018878 | ASGR2 | −1 | chr1 | 23886285 | 23885535 | 23887035 | ID3 | −1 |
| chr17 | 7017629 | 7016879 | 7018379 | ASGR2 | −1 | chr1 | 160493043 | 160492293 | 160493793 | SLAMF6 | −1 |
| chr17 | 32690250 | 32689500 | 32691000 | CCL1 | −1 | chr18 | 77160336 | 77159586 | 77161086 | NFATC1 | 1 |
| chr17 | 34329100 | 34328350 | 34329850 | CCL14 | −1 | chr5 | 35856951 | 35856201 | 35857701 | IL7R | 1 |
| chr17 | 62009696 | 62008946 | 62010446 | CD79B | −1 | chr11 | 118754475 | 118753725 | 118755225 | CXCR5 | 1 |
| chr17 | 32683471 | 32682721 | 32684221 | CCL13 | 1 | chr11 | 118764100 | 118763350 | 118764850 | CXCR5 | 1 |
| chr17 | 80275478 | 80274728 | 80276228 | CD7 | −1 | chr5 | 133450402 | 133449652 | 133451152 | TCF7 | 1 |
| chr17 | 34948228 | 34947478 | 34948978 | DHRS11 | 1 | chr5 | 133451297 | 133450547 | 133452047 | TCF7 | 1 |
| chr18 | 47901365 | 47900615 | 47902115 | SKA1 | 1 | chr5 | 133451349 | 133450599 | 133452099 | TCF7 | 1 |
| chr18 | 59992548 | 59991798 | 59993298 | TNFRSF11A | 1 | chr3 | 39323226 | 39322476 | 39323976 | CX3CR1 | −1 |
| chr18 | 52495430 | 52494680 | 52496180 | RAB27B | 1 | chr3 | 39321527 | 39320777 | 39322277 | CX3CR1 | −1 |
| chr18 | 77623668 | 77622918 | 77624418 | KCNG2 | 1 | chr3 | 39321930 | 39321180 | 39322680 | CX3CR1 | −1 |
| chr18 | 66465317 | 66464567 | 66466067 | CCDC102B | 1 | chr3 | 39322764 | 39322014 | 39323514 | CX3CR1 | −1 |
| chr18 | 66382490 | 66381740 | 66383240 | CCDC102B | 1 | chrX | 70838360 | 70837610 | 70839110 | CXCR3 | −1 |
| chr18 | 74962505 | 74961755 | 74963255 | GALR1 | 1 | chr3 | 46411633 | 46410883 | 46412383 | CCR5 | 1 |
| chr18 | 28742819 | 28742069 | 28743569 | DSC1 | −1 | chr12 | 9142137 | 9141387 | 9142887 | KLRG1 | 1 |
| chr19 | 43586820 | 43586070 | 43587570 | PSG2 | −1 | chr19 | 45971253 | 45970503 | 45972003 | FOSB | 1 |
| chr19 | 58978423 | 58977673 | 58979173 | ZNF324 | 1 | chr6 | 91006461 | 91005711 | 91007211 | BACH2 | −1 |
| chr19 | 2702707 | 2701957 | 2703457 | GNG7 | −1 | chr14 | 75988768 | 75988018 | 75989518 | BATF | 1 |
| chr19 | 38924470 | 38923720 | 38925220 | RYR1 | 1 | chr16 | 69598997 | 69598247 | 69599747 | NFAT5 | 1 |
| chr19 | 58570607 | 58569857 | 58571357 | ZNF135 | 1 | chr6 | 135502453 | 135501703 | 135503203 | MYB | 1 |
| chr19 | 47128375 | 47127625 | 47129125 | PTGIR | −1 | chr12 | 52416616 | 52415866 | 52417366 | NR4A1 | 1 |
| chr19 | 12476447 | 12475697 | 12477197 | ZNF442 | −1 | chr12 | 52445185 | 52444435 | 52445935 | NR4A1 | 1 |
| chr19 | 12949348 | 12948598 | 12950098 | MAST1 | 1 | chr1 | 25291501 | 25290751 | 25292251 | RUNX3 | −1 |
| chr19 | 49660998 | 49660248 | 49661748 | TRPM4 | 1 | chr1 | 25256770 | 25256020 | 25257520 | RUNX3 | −1 |
| chr19 | 49838657 | 49837907 | 49839407 | CD37 | 1 | chr6 | 31543344 | 31542594 | 31544094 | TNF | 1 |
| chr19 | 35939203 | 35938453 | 35939953 | FFAR2 | 1 | chr1 | 117544382 | 117543632 | 117545132 | CD101 | 1 |
| chr19 | 54785039 | 54784289 | 54785789 | LILRB2 | −1 | chr4 | 123377880 | 123377130 | 123378630 | IL2 | −1 |
| chr19 | 47523077 | 47522327 | 47523827 | NPAS1 | 1 | chr9 | 34710121 | 34709371 | 34710871 | CCL21 | −1 |
| chr19 | 39108564 | 39107814 | 39109314 | MAP4K1 | −1 | chr2 | 223289236 | 223288486 | 223289986 | SGPP2 | 1 |
| chr19 | 46171502 | 46170752 | 46172252 | GIPR | 1 | chr2 | 112895962 | 112895212 | 112896712 | FBLN7 | 1 |
| chr19 | 50922195 | 50921445 | 50922945 | SPIB | 1 | chr3 | 112052003 | 112051253 | 112052753 | CD200 | 1 |
| chr19 | 33716756 | 33716006 | 33717506 | SLC7A10 | −1 | chr11 | 36531822 | 36531072 | 36532572 | TRAF6 | −1 |
| chr19 | 14785683 | 14784933 | 14786433 | EMR3 | −1 | chr17 | 40439565 | 40438815 | 40440315 | STAT5A | 1 |
| chr19 | 6670599 | 6669849 | 6671349 | TNFSF14 | −1 | chr2 | 102759246 | 102758496 | 102759996 | IL1R1 | 1 |
| chr19 | 46526323 | 46525573 | 46527073 | PGLYRP1 | −1 | chr2 | 102608306 | 102607556 | 102609056 | IL1R2 | 1 |
| chr19 | 44529552 | 44528802 | 44530302 | ZNF222 | 1 | chr2 | 102615423 | 102614673 | 102616173 | IL1R2 | 1 |
| chr19 | 47813131 | 47812381 | 47813881 | C5AR1 | 1 | chr3 | 119278449 | 119277699 | 119279199 | CD80 | −1 |
| chr19 | 6887583 | 6886833 | 6888333 | EMR1 | 1 | chr1 | 145706861 | 145706111 | 145707611 | CD160 | −1 |
| chr19 | 52266013 | 52265263 | 52266763 | FPR2 | 1 | chr12 | 26278060 | 26277310 | 26278810 | BHLHE41 | −1 |
| chr19 | 827826 | 827076 | 828576 | AZU1 | 1 | chr2 | 12857015 | 12856265 | 12857765 | TRIB2 | 1 |
| chr19 | 851014 | 850264 | 851764 | ELANE | 1 | chr17 | 38721724 | 38720974 | 38722474 | CCR7 | −1 |
| chr19 | 55281263 | 55280513 | 55282013 | KIR2DL1 | 1 | chr1 | 8000926 | 8000176 | 8001676 | TNFRSF9 | −1 |
| chr19 | 7812420 | 7811670 | 7813170 | CD209 | −1 | chr20 | 42543504 | 42542754 | 42544254 | TOX2 | 1 |
| chr19 | 43099082 | 43098332 | 43099832 | CEACAM8 | −1 | chr3 | 137728842 | 137728092 | 137729592 | CLDN18 | 1 |
| chr19 | 35820090 | 35819340 | 35820840 | CD22 | 1 | chr11 | 35160728 | 35159978 | 35161478 | CD44 | 1 |
| chr19 | 544034 | 543284 | 544784 | GZMM | 1 | chr19 | 14492256 | 14491506 | 14493006 | CD97 | 1 |
| chr19 | 36195429 | 36194679 | 36196179 | ZBTB32 | 1 | chr2 | 187454792 | 187454042 | 187455542 | ITGAV | 1 |
| chr19 | 14889349 | 14888599 | 14890099 | EMR2 | −1 | chr2 | 187464931 | 187464181 | 187465681 | ITGAV | 1 |
| chr19 | 47835404 | 47834654 | 47836154 | C5AR2 | 1 | chr17 | 34430983 | 34430233 | 34431733 | CCL4 | 1 |
| chr19 | 55084818 | 55084068 | 55085568 | LILRA2 | 1 | chr2 | 228678570 | 228677820 | 228679320 | CCL20 | 1 |
| chr19 | 54804265 | 54803515 | 54805015 | LILRA3 | −1 | chr17 | 34417487 | 34416737 | 34418237 | CCL3 | −1 |
| chr19 | 54804221 | 54803471 | 54804971 | LILRA3 | −1 | chr6 | 167536257 | 167535507 | 167537007 | CCR6 | 1 |
| chr19 | 42300369 | 42299619 | 42301119 | CEACAM3 | 1 | chr12 | 10607215 | 10606465 | 10607965 | KLRC1 | −1 |
| chr19 | 40228668 | 40227918 | 40229418 | CLC | −1 | chr12 | 10605979 | 10605229 | 10606729 | KLRC1 | −1 |
| chr19 | 55014013 | 55013263 | 55014763 | LAIR2 | 1 | chr1 | 169680839 | 169680089 | 169681589 | SELL | −1 |
| chr19 | 55361898 | 55361148 | 55362648 | KIR3DL2 | 1 | chr1 | 169677997 | 169677247 | 169678747 | SELL | −1 |
| chr19 | 52298416 | 52297666 | 52299166 | FPR3 | 1 | chr1 | 101702444 | 101701694 | 101703194 | S1PR1 | 1 |
| chr19 | 55344173 | 55343423 | 55344923 | KIR2DS4 | 1 | chr17 | 3704537 | 3703787 | 3705287 | ITGAE | −1 |
| chr2 | 70142203 | 70141453 | 70142953 | MXD1 | 1 | chr22 | 24823530 | 24822780 | 24824280 | ADORA2A | 1 |
| chr2 | 43453748 | 43452998 | 43454498 | ZFP36L2 | −1 | chr4 | 124320665 | 124319915 | 124321415 | SPRY1 | 1 |
| chr2 | 242088850 | 242088100 | 242089600 | PASK | −1 | chr3 | 112218408 | 112217658 | 112219158 | BTLA | −1 |
| chr2 | 173420697 | 173419947 | 173421447 | PDK1 | 1 | chr1 | 37940153 | 37939403 | 37940903 | ZC3H12A | 1 |
| chr2 | 167232503 | 167231753 | 167233253 | SCN9A | −1 | chr1 | 8877524 | 8876774 | 8878274 | RERE | −1 |
| chr2 | 219646479 | 219645729 | 219647229 | CYP27A1 | 1 | chr1 | 212782012 | 212781262 | 212782762 | ATF3 | 1 |
| chr2 | 33701295 | 33700545 | 33702045 | RASGRP3 | 1 | chr10 | 3827467 | 3826717 | 3828217 | KLF6 | −1 |
| chr2 | 138721880 | 138721130 | 138722630 | HNMT | 1 | chr10 | 124768333 | 124767583 | 124769083 | IKZF5 | −1 |
| chr2 | 225266751 | 225266001 | 225267501 | FAM124B | −1 | chr10 | 135121979 | 135121229 | 135122729 | ZNF511 | 1 |
| chr2 | 113594480 | 113593730 | 113595230 | IL1B | −1 | chr10 | 35416388 | 35415638 | 35417138 | CREM | 1 |
| chr2 | 102972389 | 102971639 | 102973139 | IL18R1 | 1 | chr10 | 35415768 | 35415018 | 35416518 | CREM | 1 |
| chr2 | 219031718 | 219030968 | 219032468 | CXCR1 | −1 | chr10 | 35426709 | 35425959 | 35427459 | CREM | 1 |
| chr2 | 113542167 | 113541417 | 113542917 | IL1A | −1 | chr10 | 35456466 | 35455716 | 35457216 | CREM | 1 |
| chr2 | 37571717 | 37570967 | 37572467 | QPCT | 1 | chr10 | 35464514 | 35463764 | 35465264 | CREM | 1 |
| chr2 | 103035149 | 103034399 | 103035899 | IL18RAP | 1 | chr10 | 35484054 | 35483304 | 35484804 | CREM | 1 |
| chr2 | 48982832 | 48982082 | 48983582 | LHCGR | −1 | chr10 | 35484829 | 35484079 | 35485579 | CREM | 1 |
| chr2 | 119699742 | 119698992 | 119700492 | MARCO | 1 | chr10 | 104154354 | 104153604 | 104155104 | NFKB2 | 1 |
| chr2 | 207082748 | 207081998 | 207083498 | GPR1 | −1 | chr10 | 44144152 | 44143402 | 44144902 | ZNF32 | −1 |
| chr2 | 98330023 | 98329273 | 98330773 | ZAP70 | 1 | chr10 | 44144326 | 44143576 | 44145076 | ZNF32 | −1 |
| chr2 | 98350868 | 98350118 | 98351618 | ZAP70 | 1 | chr10 | 45495923 | 45495173 | 45496673 | ZNF22 | 1 |
| chr2 | 231989832 | 231989082 | 231990582 | HTR2B | −1 | chr10 | 64133951 | 64133201 | 64134701 | ZNF365 | 1 |
| chr2 | 162931052 | 162930302 | 162931802 | DPP4 | −1 | chr11 | 65430565 | 65429815 | 65431315 | RELA | −1 |
| chr2 | 143635067 | 143634317 | 143635817 | KYNU | 1 | chr11 | 2292182 | 2291432 | 2292932 | ASCL2 | −1 |
| chr2 | 234637773 | 234637023 | 234638523 | UGT1A8 | 1 | chr13 | 73632930 | 73632180 | 73633680 | KLF5 | 1 |
| chr2 | 102927962 | 102927212 | 102928712 | IL1RL1 | 1 | chr13 | 74569186 | 74568436 | 74569936 | KLF12 | −1 |
| chr2 | 102953716 | 102952966 | 102954466 | IL1RL1 | 1 | chr14 | 69259957 | 69259207 | 69260707 | ZFP36L1 | −1 |
| chr2 | 89157196 | 89156446 | 89157946 | IGKC | −1 | chr14 | 90085474 | 90084724 | 90086224 | FOXN3 | −1 |
| chr20 | 34203814 | 34203064 | 34204564 | SPAG4 | 1 | chr16 | 7382751 | 7382001 | 7383501 | RBFOX1 | 1 |
| chr20 | 30640064 | 30639314 | 30640814 | HCK | 1 | chr16 | 6069131 | 6068381 | 6069881 | RBFOX1 | 1 |
| chr20 | 36932525 | 36931775 | 36933275 | BPI | 1 | chr16 | 6823809 | 6823059 | 6824559 | RBFOX1 | 1 |
| chr20 | 17512014 | 17511264 | 17512764 | BFSP1 | −1 | chr17 | 11900689 | 11899939 | 11901439 | ZNF18 | −1 |
| chr20 | 17539605 | 17538855 | 17540355 | BFSP1 | −1 | chr17 | 46688379 | 46687629 | 46689129 | HOXB7 | −1 |
| chr20 | 44637547 | 44636797 | 44638297 | MMP9 | 1 | chr19 | 58694396 | 58693646 | 58695146 | ZNF274 | 1 |
| chr20 | 62367995 | 62367245 | 62368745 | LIME1 | 1 | chr19 | 44124026 | 44123276 | 44124776 | ZNF428 | −1 |
| chr20 | 3687775 | 3687025 | 3688525 | SIGLEC1 | −1 | chr19 | 19729725 | 19728975 | 19730475 | PBX4 | −1 |
| chr20 | 1638425 | 1637675 | 1639175 | SIRPG | −1 | chr19 | 53140339 | 53139589 | 53141089 | ZNF83 | −1 |
| chr20 | 896960 | 896210 | 897710 | ANGPT4 | −1 | chr2 | 208030669 | 208029919 | 208031419 | KLF7 | −1 |
| chr20 | 6182838 | 6182088 | 6183588 | TARDBPP1 | −1 | chr2 | 178129859 | 178129109 | 178130609 | NFE2L2 | −1 |
| chr21 | 43824024 | 43823274 | 43824774 | UBASH3A | 1 | chr2 | 178128617 | 178127867 | 178129367 | NFE2L2 | −1 |
| chr21 | 44847008 | 44846258 | 44847758 | SIK1 | −1 | chr2 | 71558894 | 71558144 | 71559644 | ZNF638 | 1 |
| chr22 | 39473010 | 39472260 | 39473760 | APOBEC3G | 1 | chr21 | 36421641 | 36420891 | 36422391 | RUNX1 | −1 |
| chr22 | 19701987 | 19701237 | 19702737 | 5-Sep | 1 | chr22 | 29664305 | 29663555 | 29665055 | EWSR1 | 1 |
| chr22 | 19705957 | 19705207 | 19706707 | 5-Sep | 1 | chr3 | 107241823 | 107241073 | 107242573 | BBX | 1 |
| chr22 | 30662829 | 30662079 | 30663579 | OSM | −1 | chr3 | 141868363 | 141867613 | 141869113 | TFDP2 | −1 |
| chr22 | 46663861 | 46663111 | 46664611 | TTC38 | 1 | chr4 | 26321332 | 26320582 | 26322082 | RBPJ | 1 |
| chr22 | 24384284 | 24383534 | 24385034 | GSTT1 | −1 | chr4 | 26322428 | 26321678 | 26323178 | RBPJ | 1 |
| chr22 | 40297086 | 40296336 | 40297836 | GRAP2 | 1 | chr5 | 56509948 | 56509198 | 56510698 | GPBP1 | 1 |
| chr22 | 18507325 | 18506575 | 18508075 | MICAL3 | −1 | chr6 | 139695757 | 139695007 | 139696507 | CITED2 | −1 |
| chr22 | 32150838 | 32150088 | 32151588 | DEPDC5 | 1 | chr6 | 43337181 | 43336431 | 43337931 | ZNF318 | −1 |
| chr22 | 32149936 | 32149186 | 32150686 | DEPDC5 | 1 | chr7 | 19157295 | 19156545 | 19158045 | TWIST1 | −1 |
| chr22 | 32150008 | 32149258 | 32150758 | DEPDC5 | 1 | chr8 | 17104387 | 17103637 | 17105137 | CNOT7 | −1 |
| chr22 | 27014052 | 27013302 | 27014802 | CRYBB1 | −1 | chr2 | 61108656 | 61107906 | 61109406 | REL | 1 |
| chr22 | 36556977 | 36556227 | 36557727 | APOL3 | −1 | chr3 | 71179988 | 71179238 | 71180738 | FOXP1 | −1 |
| chr22 | 36562225 | 36561475 | 36562975 | APOL3 | −1 | chr11 | 128457453 | 128456703 | 128458203 | ETS1 | −1 |
| chr22 | 39348756 | 39348006 | 39349506 | APOBEC3A | 1 | chr11 | 128392205 | 128391455 | 128392955 | ETS1 | −1 |
| chr22 | 31688514 | 31687764 | 31689264 | PIK3IP1 | −1 | chr9 | 102589009 | 102588259 | 102589759 | NR4A3 | 1 |
| chr22 | 24096655 | 24095905 | 24097405 | VPREB3 | −1 | chr5 | 142783254 | 142782504 | 142784004 | NR3C1 | −1 |
| chr22 | 45680863 | 45680113 | 45681613 | UPK3A | 1 | chr5 | 142784045 | 142783295 | 142784795 | NR3C1 | −1 |
| chr22 | 24641027 | 24640277 | 24641777 | GGT5 | −1 | chr5 | 142815077 | 142814327 | 142815827 | NR3C1 | −1 |
| chr22 | 24059610 | 24058860 | 24060360 | GUSBP11 | −1 | chr10 | 64576115 | 64575365 | 64576865 | EGR2 | −1 |
| chr22 | 25714223 | 25713473 | 25714973 | IGLL3P | 1 | chr9 | 139440314 | 139439564 | 139441064 | NOTCH1 | −1 |
| chr3 | 51975957 | 51975207 | 51976707 | RRP9 | −1 | chr7 | 17338246 | 17337496 | 17338996 | AHR | 1 |
| chr3 | 121554062 | 121553312 | 121554812 | EAF2 | 1 | chr1 | 92351787 | 92351037 | 92352537 | TGFBR3 | −1 |
| chr3 | 15563258 | 15562508 | 15564008 | COLQ | −1 | chr1 | 192544857 | 192544107 | 192545607 | RGS1 | 1 |
| chr3 | 15540379 | 15539629 | 15541129 | COLQ | −1 | chr1 | 26699266 | 26698516 | 26700016 | ZNF683 | −1 |
| chr3 | 48229846 | 48229096 | 48230596 | CDC25A | −1 | chr11 | 122709208 | 122708458 | 122709958 | CRTAM | 1 |
| chr3 | 185080908 | 185080158 | 185081658 | MAP3K13 | 1 | chr19 | 10628607 | 10627857 | 10629357 | S1PR5 | −1 |
| chr3 | 98451572 | 98450822 | 98452322 | ST3GAL6 | 1 | chr6 | 52051185 | 52050435 | 52051935 | IL17A | 1 |
| chr3 | 46283872 | 46283122 | 46284622 | CCR3 | 1 | chr1 | 206945839 | 206945089 | 206946589 | IL10 | −1 |
| chr3 | 13921618 | 13920868 | 13922368 | WNT7A | −1 | chr5 | 52083730 | 52082980 | 52084480 | ITGA1 | 1 |
| chr3 | 111260965 | 111260215 | 111261715 | CD96 | 1 | chr16 | 84651683 | 84650933 | 84652433 | COTL1 | −1 |
| chr3 | 150920979 | 150920229 | 150921729 | GPR171 | −1 | chr19 | 46651039 | 46650289 | 46651789 | IGFL2 | 1 |
| chr3 | 119316689 | 119315939 | 119317439 | PLA1A | 1 | chr19 | 46651499 | 46650749 | 46652249 | IGFL2 | 1 |
| chr3 | 11294385 | 11293635 | 11295135 | HRH1 | 1 | chr10 | 104474164 | 104473414 | 104474914 | ARL3 | −1 |
| chr3 | 11178778 | 11178028 | 11179528 | HRH1 | 1 | chr11 | 44587141 | 44586391 | 44587891 | CD82 | 1 |
| chr3 | 11196213 | 11195463 | 11196963 | HRH1 | 1 | chr11 | 3013607 | 3012857 | 3014357 | NAP1LA | −1 |
| chr3 | 11267668 | 11266918 | 11268418 | HRH1 | 1 | chr11 | 1886395 | 1885645 | 1887145 | LSP1 | 1 |
| chr3 | 48264837 | 48264087 | 48265587 | CAMP | 1 | chr12 | 6976283 | 6975533 | 6977033 | TPI1 | 1 |
| chr3 | 38029550 | 38028800 | 38030300 | VILL | 1 | chr12 | 6643093 | 6642343 | 6643843 | GAPDH | 1 |
| chr3 | 148583043 | 148582293 | 148583793 | CPA3 | 1 | chr12 | 76427712 | 76426962 | 76428462 | PHLDA1 | −1 |
| chr3 | 61547585 | 61546835 | 61548335 | PTPRG | 1 | chr5 | 149792314 | 149791564 | 149793064 | CD74 | −1 |
| chr3 | 124930243 | 124929493 | 124930993 | SLC12A8 | −1 | chr6 | 79944406 | 79943656 | 79945156 | HMGN3 | −1 |
| chr3 | 124931609 | 124930859 | 124932359 | SLC12A8 | −1 | chr7 | 2443223 | 2442473 | 2443973 | CHST12 | 1 |
| chr3 | 3152058 | 3151308 | 3152808 | IL5RA | −1 | chrX | 78622856 | 78622106 | 78623606 | ITM2A | −1 |
| chr3 | 57233983 | 57233233 | 57234733 | HESX1 | −1 | chr12 | 100378015 | 100377265 | 100378765 | ANKS1B | −1 |
| chr3 | 108541545 | 108540795 | 108542295 | TRAT1 | 1 | chr14 | 105992940 | 105992190 | 105993690 | TMEM121 | 1 |
| chrX | 17393543 | 17392793 | 17394293 | NHS | 1 | chr2 | 179672150 | 179671400 | 179672900 | TTN | −1 |
| chrX | 17653412 | 17652662 | 17654162 | NHS | 1 | | | | | | |

TABLE 3
Whole-genome (n = 116) and whole-exome (n = 39) sequencing of cell-free DNA samples
were used for PFE, training the gene expression inference model and its validation. The WGS
data were either profiled in this study (n = 30) or downloaded from Zviran et al. (EGA
accession number EGAS00001004406). Cell-free DNA from 266 subjects were profiled using EPIC-seq

| | Sequencing | | | Subjects | Cancer | Non-Cancer | |
|---|---|---|---|---|---|---|---|
| Cohort | platform | Subset | Purpose | (n) | Cases | Controls | Sources |
| Discovery | WGS | N/A | Feature discovery/selection | 117 (ciDNA: | 46 | 71 (cfDNA: | This study (n = 31); |
| | | | | 116) | | 70) | Zviran et al (n = 86) |
| Discovery/ | WES | N/A | Feature discovery/validation | 34 (39 | 6 (11 | 28 | This study |
| Validation | | | Disease detection, tumor | samples) | samples) | | |
| | | | classification, therapeutic | | | | |
| Validation | EPIC-Seq | EPIC Lung | response | 87 | 87 | N/A | This study |
| | | | Disease detection, tumor | | | | |
| | | | classification, therapeutic | | | | |
| Validation | EPIC-Seq | EPIC DLBCL | outcome | 114 | 114 | N/A | This study |
| | | | Non-Cancer Controls | | | | |
| Validation | EPIC-Seq | EPIC Control | (Specificity) | 87 | N/A. | 87 | This study |

TABLE 4
Gene groups- average expression values of genes in each group in PBMC, normalized PFE, NDR, OCF, WPS and MDS in the deep WGS sample. Each row
in this table corresponds to one group of genes (10 genes per group, n = 1,748 groups), when they are sorted according to the expression
level in PBMC. Five columns, corresponding to different fragmentomic features (PFE (this study], NDR, OCF, WPS, and MDS) are shown in this table.

| Gene group | TPM [log2] | PFE | NDR | OCF | WPS | MDS |
|---|---|---|---|---|---|---|
| B2M \|ACTB \|RPS17 \|LYZ \|RPL11 \|TYROBP \|RPS11 \|RPS24 \|RPL32 \|EEF1A1 | 13.41969556 | 0.29718 | 0.93228 | 0.18219 | −0.9882 | 0.93458 |
| PTPRC \|PPL13A \|FTH1 \|TMSB4X \|ACTG1 \|RPL27A \|PPIA \|NBPF14 \|RPS27 \|RPL10 | 12.35293801 | 0.30741 | 0.75713 | 0.14606 | −0.811 | 0.93528 |
| RPL26 \|PSAP \|RPL19 \|UBC \|PABPC1 \|TPT1 \|RPSA \|RPL21 \|RPS23 \|RPS15 | 11.75833931 | 0.30699 | 0.74303 | 0.24152 | −1.0049 | 0.94262 |
| HSPA8 \|RPL9 \|UBB \|RPS7 \|HIST2H2AC \|RPS10 \|RPS20 \|FTL \|HNRNPA1 \|RPL17 | 11.40409643 | 0.30399 | 0.66715 | 0.25751 | −0.2867 | 0.93893 |
| RPS29 \|RPL41 \|RPL36A \|TMSB10 \|CTSS \|RPS3 \|RPL30 \|RPL23A \|UBA52 \|RPS18 | 11.16378662 | 0.29977 | 0.84153 | 0.21615 | −0.9036 | 0.94219 |
| RPL4 \|RPL18 \|HLA-B \|RPS19 \|RPL35 \|HLA-DRA \|HLA-A \|HNRNPH1 \|DDX5 \|RPS6 | 10.9466588 | 0.29895 | 0.80498 | 0.10557 | −0.8493 | 0.93345 |
| RPS27A \|RPL37 \|CD44 \|HIST1H2BK \|RPL37A \|HBA1 \|RPL8 \|RPLP0 \|CD52 \|CD68 | 10.82845914 | 0.26753 | 1.07207 | −0.0057 | −0.3758 | 0.92221 |
| RPS28 \|RPL23 \|EEF1G \|NACA \|RPS15A \|ITGAL \|TXNIP \|PTMA \|FCGR2A \|BNRNPC | 10.65877859 | 0.30092 | 0.65215 | 0.31568 | −0.6523 | 0.9345 |
| HNRNPU \|VIM \|HBA2 \|RPL31 \|RPL35A \|HNRNPA2B1 \|RPL27 \|RPS25 \|NBPF9 \|EIF4B | 10.38914597 | 0.27962 | 0.71679 | 0.27431 | −0.0606 | 0.93532 |
| EEF182 \|RPL12 \|HIST2H2AA3 \|RPL38 \|CYBA \|BLA-C \|RPL34 \|RPS4X \|RPS3A \|VCAN | 10.28774652 | 0.2692 | 0.64353 | 0.17251 | −0.8469 | 0.92458 |
| RPL7A \|TPM3 \|PFDN5 \|CD74 \|RPL5 \|MYO1F \|PRKAR1A \|EVI2B \|RPS2 \|RPL36 | 10.16213584 | 0.28403 | 0.9118 | 0.13511 | −1.0526 | 0.92398 |
| RPS26 \|RPL39 \|MS4A6A \|RPS4Y1 \|LCP1 \|HIST1H2BD \|REM39 \|ARHGDIB \|MORF4L1 \|RPL15 | 10.03657535 | 0.27597 | 1.08353 | 0.03098 | −0.7649 | 0.94619 |
| HIST1H1E \|RPS9 \|FCN1 \|AHNAK \|ARPC3 \|ITM28 \|IL7R \|ATM \|HLA-E \|FXYD5 | 9.976056457 | 0.26878 | 0.9328 | 0.22498 | 0.87803 | 0.92789 |
| CD3D \|CHD2 \|GNAS \|SMN2 \|MATR3 \|LST1 \|RPL3 \|RPL18A \|RPL7 \|CALR | 9.913521076 | 0.25679 | 0.83627 | 0.14856 | −0.5697 | 0.92821 |
| S100A9 \|LAPTM4A \|CYBB \|RPL14 \|EIF4G2 \|UTRN \|SRSF5 \|SRRM2 \|NCF1 \|MVH9 | 9.811014806 | 0.2728 | 1.03779 | 0.09603 | −0.7544 | 0.93805 |
| RPS14 \|FCER1G \|HLA-DPA1 \|PICALM \|RPL6 \|CBWD3 \|SELPLG \|RPL28 \|IQGAP1 \|RPS12 | 9.726285343 | 0.28895 | 0.8917 | 0.11973 | −0.8837 | 0.93347 |
| HIST1H4J \|GAPDH \|ERAP1 \|ARID1A \|FCGR3A \|RPLP2 \|NPM1 \|HLA-DRB1 \|ANXA1 \|RAP1B | 9.644284565 | 0.26692 | 0.94629 | 0.09587 | −0.7654 | 0.92858 |
| HBB \|IL10RA \|SELL \|RPL22 \|HIST1H2BC \|RGS2 \|TMEM123 \|EIF3C \|PN2 \|MBNL1 | 9.533962396 | 0.2721 | 1.0236 | 0.10971 | 1.00949 | 0.94096 |
| ANP32A \|CASP1 \|RAC2 \|H3F3B \|GABARAP \|S100A8 \|PKM \|CASP4 \|UQCRB \|MYL6 | 9.44180296 | 0.28418 | 0.99942 | 0.11336 | 0.09819 | 0.92771 |
| COG5 \|FUS \|EEF1D \|RPS16 \|TMBIM6 \|NCF2 \|TPI1 \|HLA-F \|LUC7L3 \|RNF213 | 9.385680769 | 0.30087 | 0.88847 | 0.14267 | −1.0075 | 0.92447 |
| C1orf162 \|HLA-DRB5 \|HLA-DPB1 \|ANKRD44 \|CORO1A \|FLNA \|RPS5 \|IFI30 \|GNLY \|RPS8 | 9.256405143 | 0.26971 | 1.00586 | 0.06115 | −0.8441 | 0.91882 |
| AP1S2 \|BOLA2B \|IRF8 \|ZEB2 \|RPL24 \|PLAC8 \|PTPN6 \|MAX \|FAU \|CIITA | 9.190274726 | 0.25006 | 1.10376 | 0.01799 | −0.453 | 0.938 |
| CARD16 \|SUMO2 \|DDX3Y \|SERPINA1 \|BAZ2A \|CD53 \|RPL10A \|ARRB2 \|MYL12A \|FOS | 9.135520556 | 0.28699 | 0.80478 | 0.22665 | −0.2013 | 0.92855 |
| RPL13 \|DDX17 \|PSMEL \|HLA-DQA1 \|SRGN \|SRSF11 \|EEF2 \|PTEN \|ALDOA \|ANXA2 | 9.096119407 | 0.28062 | 0.81055 | 0.16591 | −0.773 | 0.92881 |
| HCLS1 \|OAZ1 \|P4HB \|PPT1 \|CANX \|SON \|KLF6 \|PCBP2 \|TPM4 \|DNM2 | 9.043086807 | 0.29174 | 0.83468 | 0.11224 | −0.4263 | 0.93225 |
| IST1 \|RSRC2 \|ARPC2 \|NPC2 \|CASP8 \|YWHAZ \|PPP1R12A \|SET \|AKAP13 \|BTF3 | 8.976955876 | 0.30008 | 0.72547 | 0.15184 | 0.41797 | 0.92677 |
| PHF21A \|S100A4 \|SF3B1 \|PDCD4 \|CD46 \|ATP6AP2 \|NDUFA13 \|HSD17B4 \|NCL \|ATP5L | 8.916477642 | 0.29812 | 0.88908 | 0.09576 | −0.7738 | 0.9258 |
| SAMHD1 \|TUBA1B \|TARDEP \|TGOLN2 \|CAP1 \|IFITM2 \|TAB2 \|EWSR1 \|ITGB2 \|EIF3L | 8.861573198 | 0.28954 | 0.90925 | 0.06438 | −0.9114 | 0.92515 |
| CAPN2 \|UBAP2L \|AGAP4 \|LEF1 \|AOAH \|NFKBIA \|RBM5 \|HSPA9 \|ATXN7 \|HIF1A | 8.818490813 | 0.26347 | 0.92019 | 0.14038 | −0.9259 | 0.9357 |
| PPIB \|TPP1 \|PGK1 \|LCP2 \|RANBP2 \|PFN1 \|CD247 \|EIF3E \|PARK7 \|GRN | 8.792875437 | 0.28017 | 0.88376 | 0.17436 | −0.709 | 0.94282 |
| EMP3 \|ACADVL \|SMG1 \|SORL1 \|M6PR \|WNK1 \|SNRNP70 \|CIRBP \|ATP51 \|RAB7A | 8.755511093 | 0.29618 | 0.84308 | 0.05274 | 0.50102 | 0.91281 |
| DGKA \|ARHGEF1 \|GLUL \|ITK \|RPLP1 \|HSP90AA1 \|HK1 \|DOCK8 \|HNRNPK \|EIF3K | 8.72894663 | 0.26922 | 0.92771 | 0.08798 | −0.0448 | 0.93601 |
| CBWD2 \|LDHA \|GIMAP4 \|CD3E \|BTN3A2 \|CAPZA1 \|COX6A1 \|ADAM10 \|TUBA1A \|GTF2I | 8.705236862 | 0.26881 | 0.88379 | 0.14779 | −0.9431 | 0.94174 |
| IK \|SPDYE2 \|CPVL \|ARHGAP26 \|ACTR2 \|COLGA8A \|APLP2 \|DOCK2 \|STAT1 \|PPBP | 8.658733786 | 0.26358 | 1.07643 | 0.01522 | −0.3036 | 0.93475 |
| CDC42 \|AKNA \|ITGB1 \|PLBD1 \|MACF1 \|IL32 \|ZC3H11A \|CAPNS1 \|CCL5 \|NOP10 | 8.624050521 | 0.22402 | 1.16125 | 0.03751 | 0.31421 | 0.92959 |
| CAST \|LTA4H \|LIMS1 \|DAZAP2 \|HMGN1 \|HLA-DQB1 \|DDX3X \|SMN1 \|CD63 \|RNASET2 | 8.576438619 | 0.29348 | 0.94324 | 0.16824 | −0.7162 | 0.9186 |
| NAMPT \|G3BP2 \|PHC3 \|CYTH1 \|MS4A1 \|NONO \|CTSA \|CLEC12A \|LSP1 \|BRD2 | 8.547429492 | 0.24526 | 1.10255 | 0.0279 | −0.6094 | 0.92944 |
| DDX39B \|SOD2 \|OGT \|EVL \|CD37 \|ACTN4 \|CFL1 \|CSDE1 \|ST13 \|PARP8 | 8.52105666 | 0.27622 | 0.79859 | 0.12169 | −0.8105 | 0.93163 |
| ATP6V1B2 \|LAPTM5 \|LILRA5 \|ACTR3 \|AIF1 \|EIE5 \|CCNL1 \|FNDC3B \|ID2 \|WDR1 | 8.488655962 | 0.2872 | 0.70353 | 0.1855 | −0.8702 | 0.91418 |
| PRKCH \|WAC \|HIST1H3D \|DNMT1 \|TCF7 \|POLR2J \|CSF3R \|SMAD3 \|ANXA5 \|QARS | 8.468832045 | 0.28316 | 0.96982 | 0.06682 | 0.37116 | 0.91472 |
| SFPQ \|EDF1 \|NAP1L1 \|PDIA3 \|SLC44A2 \|HSP90AB1 \|INPP5D \|HIST1H2BI \|ATP5B \|IFI16 | 8.43756902 | 0.28256 | 0.89354 | 0.04806 | −0.8728 | 0.92586 |
| SOD1 \|NCOA4 \|GMFG \|SDCBP \|PRNP \|TALDO1 \|NOMO1 \|CST3 \|LRRFIP1 \|PIAS1 | 8.4054797 | 0.27868 | 0.84588 | 0.21092 | −0.8468 | 0.92348 |
| HIST1H2AE \|IFNGR2 \|H2AFY \|ZNF207 \|TAF15 \|FGL2 \|PLCB2 \|LRRC37A2 \|AK2 \|YBX1 | 8.368429753 | 0.28281 | 0.71707 | 0.18551 | −0.9094 | 0.93515 |
| SF1 \|SF3A1 \|FOXP1 \|PABPN1 \|MKRN1 \|ADD3 \|SPG11 \|UBE2D3 \|PRPF8 \|BCLAF1 | 8.337213504 | 0.3037 | 0.6797 | 0.18077 | −1.0107 | 0.93468 |
| DIAPH1 \|GNB1 \|CNOT2 \|SLTM \|SNURF \|STXBP2 \|USP15 \|PIK3R1 \|STAT6 \|NDRG1 | 8.304156614 | 0.29146 | 0.85818 | 0.12053 | −0.7016 | 0.92341 |
| TRIP12 \|HEXB \|EIF3H \|CD55 \|EIF4A1 \|HSP90B1 \|SRGAP2 \|EIF4G1 \|SOS1 \|CBWD5 | 8.28111511 | 0.28034 | 0.82323 | 0.27446 | −0.6461 | 0.92935 |
| RAB8B \|EIF3M \|BTN3A3 \|NDUFB1 \|LILRB2 \|S100A11 \|SPTAN1 \|CHKB \|SMAD4 \|BSPA1A | 8.250512559 | 0.27685 | 0.76125 | 0.25842 | −0.9943 | 0.93159 |
| PNRC1 \|MYD88 \|PRELID1 \|PPIG \|HIST1H1C \|MEF2C \|LARP1 \|GDI2 \|HIST2H2BF \|ENO1 | 8.232829316 | 0.28167 | 0.95919 | 0.12193 | −0.0243 | 0.93358 |
| WAS \|TMA7 \|GIMAP7 \|LITAF \|HNRNPA3 \|SYNE2 \|RPS6KA3 \|ATG3 \|SLAIN2 \|XPO6 | 8.216214199 | 0.28734 | 0.83018 | 0.14037 | −0.7436 | 0.92707 |
| ANXA6 \|SULT1A3 \|SRRM1 \|PLP2 \|EPB41 \|ITGA4 \|SH3BP2 \|POLR2J2 \|ATP5J2 \|BIN2 | 8.181473744 | 0.28402 | 1.01407 | 0.04603 | −0.7804 | 0.91505 |
| SMCHD1 \|TRIOBP \|RPL29 \|HADHB \|CTSH \|SP110 \|PTPN12 \|PPP2R5C \|PRKCSH \|CYTH4 | 8.158876442 | 0.28332 | 0.80806 | 0.12713 | −1.0325 | 0.9164 |
| GBP2 \|NBPF10 \|CREBBP \|SP100 \|NCKAP1L \|GBP3 \|TUBA4A \|HIST1H2AK \|ZRANB2 \|HNRNPD | 8.145225927 | 0.25768 | 1.0389 | 0.08373 | −0.4725 | 0.94155 |
| PGD \|SEC62 \|HIST2H2BE \|MDM4 \|DMTF1 \|AMY2B \|SMAD2 \|MGEA5 \|AGAP6 \|FLI1 | 8.118557283 | 0.28757 | 0.89863 | 0.13051 | 0.17313 | 0.93654 |
| RGPD8 \|PAFAH1B1 \|CD36 \|VCL \|WARS \|ARL17B \|PDHA1 \|MSN \|TCOF1 \|TOMM7 | 8.094741227 | 0.27899 | 0.73462 | 0.11274 | −1.0105 | 0.93084 |
| GBP1 \|ADAR \|ARHGAP9 \|C11orf58 \|KRTCAP2 \|SB3KBP1 \|RPS13 \|GZMH \|HP1BP3 \|CD48 | 8.068268519 | 0.25217 | 0.89911 | 0.1982 | −0.3913 | 0.93323 |
| FGR \|TRA2B \|JAK1 \|ADD1 \|CYTIP \|ASAH1 \|AHSA1 \|VMP1 \|NOTCH2 \|NIPBL | 8.050427494 | 0.29667 | 0.85837 | 0.21312 | 0.94666 | 0.92956 |
| NOMO2 \|RABGAP1L \|CAPRIN1 \|CXCR4 \|RBM25 \|GSTK1 \|CALM1 \|GIGYF2 \|SKP1 \|ARPC1B | 8.0237442 | 0.29646 | 0.71842 | 0.27511 | −0.8275 | 0.92781 |
| CNOT1 \|CCT5 \|TMC6 \|LILRB3 \|COX6B1 \|LGALS9 \|YY1AP1 \|EIF4A2 \|FYN \|BCL9L | 8.003167237 | 0.27992 | 0.86057 | 0.12821 | −0.087 | 0.9148 |
| HERPUD1 \|PRRC2C \|CCZ1B \|TBXAS1 \|CDC42SE1 \|STK10 \|CDKN1B \|SLC7A7 \|MNDA \|FMNL1 | 7.978900455 | 0.28747 | 0.83991 | 0.14078 | −0.3447 | 0.9296 |
| CSNK1D \|FAM192A \|APEX1 \|CTSC \|OPA1 \|EIF253 \|DNAJC8 \|CRIP1 \|MFSD1 \|CR1 | 7.959619765 | 0.27361 | 0.79657 | 0.16918 | −0.2995 | 0.92581 |
| NAGK \|ZNF91 \|TRAF3IP3 \|PCM1 \|RHOA \|SYNE1 \|MED13L \|LGALS2 \|SLC12A6 \|PLEKHA2 | 7.94538537 | 0.26242 | 0.98947 | 0.03043 | −0.2152 | 0.92702 |
| HNRNPH3 \|OS9 \|TLK1 \|CSNK1A1 \|RBM3 \|EIF3A \|COX4I1 \|TMED10 \|SMAP2 \|RASGRP2 | 7.923930803 | 0.31337 | 0.74206 | 0.14077 | −0.2886 | 0.92241 |
| MED15 \|H3F3A \|CAT \|STAG2 \|NOP56 \|MYL12B \|SNAP23 \|CBWD1 \|HLA-DMA \|VPS13C | 7.908493591 | 0.2949 | 0.94599 | 0.15964 | −0.9005 | 0.94239 |
| PAPOLA \|EIF5A \|MCL1 \|DEK \|HSPD1 \|ZFAND5 \|ANP32B \|STAT3 \|PTPRA \|TKT | 7.882822461 | 0.30081 | 0.84389 | 0.15185 | −0.0191 | 0.91526 |
| FIPI1L1 \|DUSP6 \|SMARCA2 \|AKT2 \|NCSTN \|SAT1 \|UCP2 \|PIP4K2A \|NSD1 \|TMPO | 7.862056184 | 0.29498 | 0.77717 | 0.10354 | −0.8839 | 0.93596 |
| SUN2 \|ITCH \|TGE81 \|ARHGAP15 \|G8A \|TCF25 \|SSR2 \|NSF \|E2F4 \|CD3G | 7.833636718 | 0.26426 | 0.72128 | 0.27478 | −0.5109 | 0.92751 |
| SMARCE1 \|PTP4A2 \|SERF2 \|RAB10 \|CTAGE5 \|PDCD6IP \|SRSF3 \|MTDH \|SIRPB1 \|TRAM1 | 7.806996781 | 0.29196 | 0.70352 | 0.18868 | −0.9921 | 0.93164 |
| GZMB \|RFTN1 \|PAK1 \|RBM17 \|TCF12 \|ARL6IP5 \|TMC8 \|USF2 \|SRP14 \|TBCA | 7.786117057 | 0.27043 | 0.9387 | 0.19805 | 0.68101 | 0.92412 |
| RABSA \|HADHA \|CALM3 \|TAGLN2 \|GRB2 \|LDHB \|EP300 \|TTC14 \|ANKRD12 \|S100A6 | 7.769440253 | 0.30002 | 0.88216 | 0.05143 | −0.4665 | 0.92472 |
| LYN \|SAFB \|NMT1 \|UBE2L6 \|HIST1H2BJ \|BTN3A1 \|CLEC2B \|NDUFV1 \|TECR \|SLC2A3 | 7.739926907 | 0.29354 | 0.72676 | 0.18328 | −0.916 | 0.93893 |
| EFTUD2 \|QKI \|LSM14A \|PSIP1 \|SATB1 \|TNRC6A \|CLK3 \|CLTC \|SSR1 \|STIM1 | 7.717470993 | 0.30489 | 0.73808 | 0.15657 | −0.9425 | 0.92245 |
| SLC9A3R1 \|CFLAR \|FAM49B \|SUB1 \|GON4L \|ATRX \|IDH3B \|RASSF5 \|BEXA \|TGFBR3 | 7.701343469 | 0.29179 | 0.89291 | 0.18299 | 0.12546 | 0.92751 |
| PET100 \|CAPZB \|MAZ \|EXOC4 \|FAM120A \|AES \|CD96 \|SSR4 \|DYNC1H1 \|S100A12 | 7.689703959 | 0.26899 | 0.863 | 0.11282 | −0.8242 | 0.92444 |
| DBNL \|DPYD \|FNBP1 \|RBM23 \|CDK17 \|DAP3 \|GNAI2 \|APP \|CTNNA1 \|NLRC5 | 7.673865163 | 0.29545 | 0.60369 | 0.3093 | −0.8899 | 0.9165 |
| ETFA \|RBM33 \|XRCC5 \|SND1 \|PLEK \|DOCK10 \|DYM \|RBM6 \|UXT \|WASF2 | 7.657924919 | 0.28302 | 0.87723 | 0.1642 | −0.5654 | 0.93484 |
| PSMD4 \|SDHB \|KPNB1 \|HIST1H1D \|SFSWAP \|PTBP1 \|WIPF1 \|TUBB1 \|HMGN2 \|BAZ2B | 7.641301478 | 0.28504 | 1.05907 | 0.12709 | −0.4333 | 0.92884 |
| CDK5RAP3 \|PSEN1 \|ZNF638 \|RNASEB2B \|CLIC1 \|KLRK1 \|CALM2 \|ELP2 \|ATF4 \|GALT | 7.621705249 | 0.29241 | 0.75311 | 0.21454 | −0.3351 | 0.93692 |
| VASP \|RNASEK \|MAP4K1 \|HIST1H4F \|G3BP1 \|HDLBP \|SLC25A3 \|AGAP9 \|RAF1 \|CCL4 | 7.607892473 | 0.28284 | 1.04296 | 0.15371 | −0.8014 | 0.9247 |
| ATXN10 \|LENG8 \|RSL1D1 \|ATP1A1 \|HIST1B2BN \|PLCG1 \|CFP \|NDUFS3 \|NUP62 \|VAPA | 7.596830348 | 0.29143 | 0.85524 | 0.07891 | −0.2877 | 0.91899 |
| CRBN \|NCOR1 \|CLEC2D \|MIA3 \|NOMO3 \|DBI \|LYST \|SP140L \|SLC11A1 \|MAP4K4 | 7.581689686 | 0.27112 | 0.88946 | 0.12495 | −0.6757 | 0.93534 |
| GIMAP5 \|GLUD1 \|ZNF384 \|PTPN11 \|RSF1 \|LAMTOR2 \|DRAM2 \|PNN \|LILRA2 \|NIN | 7.565073675 | 0.26766 | 0.74788 | 0.12948 | −0.2315 | 0.92055 |
| TCERG1 \|ATP11B \|CYFIP2 \|FRG1 \|FNBP4 \|RBM8A \|BPTF \|CORO1B \|DICER1 \|WSB1 | 7.551233669 | 0.2992 | 0.54154 | 0.28649 | −0.2239 | 0.93157 |
| HAX1 \|YWHAE \|SYMPK \|DKC1 \|SDHA \|MYO15B \|CALCOCO2 \|GLRX \|PTPN4 \|DNAJB6 | 7.540131212 | 0.28692 | 0.93796 | 0.15287 | −0.6618 | 0.9176 |
| ANKRD36 \|TMEM63A \|DDX6 \|RBMX \|FXR1 \|CTNNB1 \|MPRIP \|SMARCB1 \|NUMB \|BTK | 7.52555735 | 0.28592 | 0.80775 | 0.2615 | −0.0434 | 0.9158 |
| SULF2 \|SP140 \|PNKP \|NDUFS5 \|STK17B \|CLASRP \|ATP5G2 \|KDM5A \|NEKBIZ \|ARGLU1 | 7.50676198 | 0.28919 | 0.79714 | 0.20942 | −0.9576 | 0.93322 |
| MAN2B1 \|CD2 \|SH3BGRL \|PARP1 \|PF4 \|ZFP36L1 \|RCOR3 \|BRD7 \|TRAPPC10 \|EIF2B5 | 7.485968155 | 0.28044 | 0.77045 | 0.20578 | −0.7898 | 0.93439 |
| FKBP1A \|MYO9B \|ZFAND3 \|NDUFA1 \|TMEM71 \|SHOC2 \|IL2RG \|LGALS8 \|ARHGAP25 \|MPDU1 | 7.471944413 | 0.28782 | 0.9511 | 0.03284 | −0.506 | 0.93763 |
| ELF1 \|SDHD \|CSTA \|VPS41 \|SERBP1 \|RASA4 \|SF3B3 \|VDAC3 \|PRPF3 \|SREK1 | 7.457938681 | 0.26735 | 1.02551 | 0.17686 | −0.8297 | 0.94731 |
| KARS \|ITGAX \|CTSW \|POLR2B \|KLRC2 \|IFITM1 \|SIDT2 \|ANKHD1 \|PRPF4B \|STK4 | 7.437957082 | 0.27057 | 0.87018 | 0.17312 | −0.5726 | 0.92458 |
| UBE3A \|ZRSR2 \|EIF3D \|UQCRC2 \|SPTLC2 \|LPP \|IMPDH1 \|PRR13 \|HIST1H2BO \|PARVG | 7.422154384 | 0.27353 | 1.07514 | 0.05212 | −0.6103 | 0.93856 |
| NUB1 \|PNPLA6 \|FAM107B \|MBD5 \|ELK4 \|BAZ1A \|LDLRAP1 \|SDHC \|RAB2A \|HNRNPUL1 | 7.40958124 | 0.28304 | 0.94138 | 0.09673 | −0.7976 | 0.93302 |
| TRIM22 \|CHD7 \|PRPF40A \|CLK1 \|RPL36AL \|LMAN2 \|NDUFS2 \|BNIP3L \|AFF1 \|RBPJ | 7.394005255 | 0.29759 | 0.84159 | 0.14252 | −0.8246 | 0.9287 |
| ATF7IP \|PIK3CD \|BIRC6 \|CCDC12 \|MLH1 \|NUCB1 \|STAT5B \|PIK3AP1 \|XPO1 \|ACAP1 | 7.380619847 | 0.28238 | 0.91244 | 0.07129 | 0.15312 | 0.91246 |
| ARPP19 \|SF3A3 \|RPE \|TINF2 \|ZNF451 \|PPP3CC \|PRF1 \|PSMB3 \|POM121C \|IQGAP2 | 7.365584835 | 0.26266 | 0.89007 | 0.14017 | −0.8766 | 0.93062 |
| NDE1 \|CREB5 \|TBC1D3B \|NBPF15 \|IL6R \|RAB6A \|TAF10 \|ILK \|HNRNPM \|A2M | 7.351975967 | 0.25588 | 0.85653 | 0.09512 | 0.5586 | 0.94065 |
| USP25 \|IRF1 \|STIP1 \|UBE2D2 \|PILRA \|CELF2 \|GPR89A \|TRAPPC1 \|GLS \|CLEC7A | 7.338211794 | 0.27356 | 0.74591 | 0.17951 | −0.4012 | 0.92086 |
| CDC37 \|ILF3 \|UNC13D \|RNF13 \|R1C8A \|AMZ2 \|PTCD3 \|RAB27A \|PRRC2B \|ZAP70 | 7.321134585 | 0.25199 | 1.02271 | 0.11909 | −0.8386 | 0.91935 |
| GDI1 \|CORO1C \|GSTP1 \|DNM1L \|TGFBI \|SLC38A1 \|CCNI \|SSH2 \|RUNX1 \|XRN1 | 7.30969546 | 0.27861 | 0.75953 | 0.19687 | −1.082 | 0.92209 |
| PPP6R3 \|EPS15 \|CLINT1 \|SETX \|HIST1H2AJ \|TOR1AIP1 \|PQBP1 \|C9orf78 \|ENTPD4 \|PRKAG2 | 7.301041039 | 0.30316 | 0.78692 | 0.20391 | −0.2213 | 0.93313 |
| HM13 \|TXK \|SLIRP \|TIA1 \|ATP2A2 \|CDYL \|EIF4E2 \|RNPC3 \|ACAP2 \|UBL5 | 7.289604093 | 0.2848 | 0.78049 | 0.20041 | −0.9725 | 0.94157 |
| MAPKAPK3 \|SQSTM1 \|CPSF7 \|AGAP5 \|ERGIC2 \|PRKCB \|USP4 \|RNF130 \|CCAR1 \|FGD3 | 7.279256708 | 0.28205 | 0.84647 | 0.11912 | −0.5042 | 0.92566 |
| CMPK1 \|HIST1H2AL \|ITGA6 \|CD164 \|IL6ST \|USP7 \|TAOK1 \|PSMB4 \|PSME2 \|TNFRSF1A | 7.270003333 | 0.30589 | 0.6883 | 0.19091 | −0.3361 | 0.92198 |
| HIST1B2AC \|VCP \|F8XO7 \|SEC63 \|CSK \|SLC7A6 \|SRI \|ETS1 \|PILRB | 7.256093407 | 0.28982 | 0.80692 | 0.15462 | −0.9237 | 0.93034 |
| PLEKHM1 \|PLEC \|STX16 \|ERGIC3 \|C6orf48 \|PHKB \|GGA2 \|DGKZ \|ELAC2 \|GSR | 7.244097865 | 0.29154 | 0.75309 | 0.24024 | −0.5591 | 0.91398 |
| ERCC1 \|POLR2A \|TC2N \|HIST1H4E \|GTF3A \|TET2 \|ATXN2 \|IFITM3 \|TSC22D3 \|DDX42 | 7.233307374 | 0.28581 | 1.14792 | −0.0166 | −0.2867 | 0.92679 |
| RNF10 \|OPTN \|SUMO1 \|SNX29 \|GPCPD1 \|CSNK2A1 \|NAP1L4 \|MSI2 \|CLU \|DECR1 | 7.218415998 | 0.27322 | 0.70912 | 0.27782 | −0.7016 | 0.91872 |
| EFHD2 \|TRIM33 \|GRAP2 \|YTHDC1 \|SNRPG \|PSMF1 \|RSU1 \|ARID4A \|SEC31A \|PNISR | 7.209299513 | 0.29567 | 0.64738 | 0.27239 | −0.7065 | 0.94245 |
| NEATC3 \|SAP30BP \|AKAP9 \|CSF1R \|FBXO38 \|PSTPIP1 \|ATP2C1 \|MTSS1 \|INTS8 \|SGSH | 7.197933774 | 0.25848 | 0.9134 | 0.09626 | −0.1134 | 0.9291 |
| CTSD \|PTK2B \|RAPGEF2 \|HERC3 \|MYCBP2 \|SNAP29 \|MTRR \|ATG16L2 \|HNRNPR \|CCDC88C | 7.190932777 | 0.29189 | 0.67714 | 0.18098 | −0.0611 | 0.92621 |
| OSBPL8 \|KDM5C \|KCNAB2 \|DLD \|GPS2 \|NBPF11 \|MAP3K1 \|ZC3H14 \|VPS8 \|TMEM59 | 7.179711443 | 0.28049 | 0.87317 | 0.0738 | −0.4884 | 0.92824 |
| PEA15 \|ARRB1 \|FAM45A \|OSTF41 \|LARS \|TSPAN14 \|NOP58 \|TNEAIP2 \|ITGAM \|CNOT6L | 7.168846976 | 0.2929 | 0.60295 | 0.289 | 0.28345 | 0.91478 |
| PPP1CB \|NDUFA11 \|TLE4 \|HCK \|GORASP2 \|NME2 \|ARHGDIA \|TREM1 \|PXN \|MPEG1 | 7.157275342 | 0.2719 | 0.8924 | 0.1457 | 0.45499 | 0.93122 |
| BMP2K \|SNX2 \|HIST1H2BM \|UBA2 \|GPI \|ENTPD1 \|TRPS1 \|XRCC6 \|TCIRG1 \|TTC3 | 7.144775348 | 0.27558 | 0.93807 | 0.1016 | 0.20185 | 0.93622 |
| LRRC37A \|TAP1 \|HUWE1 \|VNS1ABP \|LMAN1 \|JMID1C \|SNRPD2 \|MARK3 \|HBG2 \|AUTS2 | 7.136037952 | 0.27977 | 0.74025 | 0.00858 | −0.1198 | 0.92966 |
| UBE2E1 \|NXF1 \|PUM2 \|PDCD10 \|PA2G4 \|ANXA7 \|BABAM1 \|KIAA0907 \|FAM120B \|EIF2A | 7.127580427 | 0.28703 | 0.75853 | 0.12302 | −0.8889 | 0.93631 |
| NEDD8 \|KIF2A \|CCM2 \|FUBP1 \|UQCRH \|SENP6 \|CCDC69 \|ZEP36 \|CD79B \|CCT7 | 7.117601204 | 0.29039 | 0.94115 | 0.19028 | −0.0926 | 0.91904 |
| GLIPR1 \|EIF3F \|RBL2 \|PCNP \|BTN2A1 \|LIMD2 \|TAP2 \|ASAP1 \|NPEPPS \|FAM1I1A | 7.110497427 | 0.24317 | 0.97994 | 0.20326 | −0.5272 | 0.94093 |
| RUFY1 \|HDAC1 \|THEMIS2 \|NBPF1 \|GARS \|BZW1 \|CASC3 \|RPS6KA1 \|ABLIM1 \|CBLB | 7.095617612 | 0.26195 | 0.81667 | 0.04793 | −0.0754 | 0.92827 |
| TOX4 \|SEC23B \|RER1 \|HIPK3 \|EMC3 \|ZYX \|SPG7 \|PSMB9 \|COPA \|MAP2K3 | 7.079007803 | 0.30891 | 0.61372 | 0.23175 | −0.611 | 0.92126 |
| GPX4 \|GBP5 \|PI4KA \|TUFM \|ASXL1 \|SPI1 \|STK24 \|HMGN3 \|NR3C1 \|ATP5F1 | 7.070806375 | 0.27568 | 0.83102 | 0.15562 | −0.6328 | 0.92404 |
| RASA1 \|ALG13 \|GZMA \|NDUFA3 \|TANK \|MARS \|ANKRD11 \|THRAP3 \|RBBP4 \|NUP50 | 7.061991642 | 0.29191 | 0.58584 | 0.30274 | −0.7838 | 0.93659 |
| PIK3R5 \|TES \|CDC14A \|CNBP \|SNRNP200 \|EHMT1 \|HDGF \|NDUFA10 \|CKLF \|KLF12 | 7.051650973 | 0.28266 | 0.85834 | 0.16371 | 0.16626 | 0.93334 |
| SPG21 \|NKTR \|VPS13B \|DDX23 \|SMARCC2 \|TRA2A \|NUMA1 \|CPNE1 \|SYNCRIP \|UBA1 | 7.038513465 | 0.29001 | 0.6947 | 0.25547 | −0.8828 | 0.93192 |
| DYNLL1 \|HIST1H2BL \|ANKRD13A \|MTMR14 \|PTBP3 \|MSL3 \|ERGIC1 \|GGNBP2 \|CCND3 \|COX7A2 | 7.026921131 | 0.28454 | 0.8167 | 0.14884 | −0.2134 | 0.93359 |
| MDM2 \|ITSN2 \|FAM50A \|BBS2 \|RAN \|SP3 \|ESYT1 \|MS4A7 \|LGALS1 \|RNF19A | 7.015528719 | 0.27083 | 1.07284 | 0.06126 | 0.70926 | 0.92623 |
| IGF1R \|UBE2I \|NDUFB9 \|SPOCK2 \|SECISBP2 \|EIF2AK3 \|UBA7 \|RFWD2 \|HIST1H31 \|SERPINB1 | 7.002708733 | 0.29168 | 0.96917 | 0.20361 | −0.5235 | 0.93311 |
| ANKRD17 \|SETD5 \|NEMF \|MT2A \|HIST1H2BE \|CYLD \|IRF9 \|USP3 \|NECAP2 \|SAR1A | 6.988647765 | 0.2987 | 0.82683 | 0.04178 | 1.11016 | 0.93087 |
| FLII \|TLN1 \|HMGB1 \|PSMA6 \|STAT2 \|DOK2 \|FAM76B \|DCXR \|C60x162 \|PANK2 | 6.976727374 | 0.30735 | 0.70706 | 0.14286 | −0.6076 | 0.92893 |
| KIAA1109 \|NDUFS6 \|MYO5A \|RAB18 \|SEC23A \|KLC1 \|LIPA \|SLC38A2 \|KDM6A \|ARHGEF2 | 6.965534703 | 0.29582 | 0.78393 | 0.19282 | −0.4799 | 0.92611 |
| SSBP3 \|SPCS1 \|ABCF1 \|NAAA \|CDK11A \|SMARCC1 \|ARHGAP4 \|SAFB2 \|PACS1 \|SETD3 | 6.956811322 | 0.30503 | 0.68504 | 0.18012 | −0.2846 | 0.90493 |
| LBR \|DDX24 \|KLRD1 \|TPP2 \|TSG101 \|MIER1 \|SUPT4H1 \|RBM4 \|UBP1 \|STX7 | 6.948278503 | 0.2927 | 0.79442 | 0.15878 | −0.4809 | 0.9487 |
| PRMT2 \|PAPD4 \|CREB1 \|MAN1B1 \|MADD \|CDV3 \|C17orf49 \|AP2M1 \|PCYT1A \|CAPZA2 | 6.937901225 | 0.31946 | 0.54806 | 0.22294 | −0.8459 | 0.93198 |
| MGST3 \|VPS35 \|TMCO1 \|WDFY1 \|GTF2IRD2B \|SCFD1 \|IL16 \|CARD8 \|DUSP1 \|HCST | 6.927144302 | 0.27406 | 1.04328 | 0.07434 | −0.2384 | 0.92729 |
| RPS21 \|COX7C \|CLDND1 \|N4BP2L2 \|RAB3GAP2 \|PPIP5K1 \|ELMO1 \|ARID4B \|C14orf2 \|NDUFS1 | 6.918761712 | 0.29289 | 0.83153 | 0.181 | −0.547 | 0.93836 |
| TCF4 \|YWHAQ \|CHD4 \|HIST1H2BF \|GLG1 \|FLOT1 \|NT5C2 \|PPM1 \|USP8 \|TBC1D15 | 6.911048422 | 0.28119 | 0.72226 | 0.25705 | −0.7572 | 0.94254 |
| VAMP8 \|KDM4C \|CD6 \|RAP1GAP2 \|TAXBP1 \|TGIF1 \|SEMA4D \|IL4R \|CHD6BTG1 | 6.904647976 | 0.26604 | 0.85561 | 0.05849 | −0.6549 | 0.91761 |
| UFC1 \|PHB2 \|TRIM28 \|CPPED1 \|AAK1 \|DOCK11 \|ZCCHC11 \|ARRDC3 \|SHKBP1 \|ARIH2 | 6.900623896 | 0.31269 | 0.71207 | 0.15364 | −0.7238 | 0.93176 |
| SCP2 \|SCPEP1 \|NARS \|EIF1 \|SYNRG \|CBL \|HPS1 \|GNL2 \|KRIT1 \|CTC1 | 6.893793914 | 0.28462 | 0.73134 | 0.27113 | −0.5974 | 0.93302 |
| CCT2 \|NDUFV2 \|AP3B1 \|PAIP2 \|DENND2D \|DGUOK \|C14orf166 \|ATP2A3 \|MAML1 \|EPB41L3 | 6.887146818 | 0.27757 | 0.77553 | 0.16785 | −0.3068 | 0.93132 |
| NUP214 \|EM8 \|NSMAF \|SGK1 \|NELL2 \|UBR1 \|FTO \|IL17RA \|MAN2C1 \|METTL9 | 6.878650861 | 0.26161 | 0.92573 | 0.13833 | −0.9794 | 0.93881 |
| LPXN \|ZFR \|EIF2B1 \|CCDC66 \|LUC7L2 \|KDM5D \|CHD8 \|SNRPD1 \|TACC1 \|ATG16L1 | 6.872038437 | 0.28647 | 0.95493 | 0.21174 | −0.4827 | 0.93785 |
| ZNF644 \|EML4 \|CASP2 \|SPTLC1 \|UBE2B \|PSMB8 \|IFNAR2 \|DENND4B \|SP1 \|GIT2 | 6.862470152 | 0.29628 | 0.50403 | 0.22752 | −0.1014 | 0.92809 |
| ITGA5 \|PTPN22 \|ARAP1 \|XPNPEP1 \|KDM3B \|UGGT1 \|TEP1 \|DNAJC10 \|CASCA \|MTR | 6.853186161 | 0.2851 | 0.73797 | 0.14865 | −0.765 | 0.92746 |
| RNF149 \|CHMP4B \|HDAC4 \|BBX \|CDK5RAP2 \|PPP1R2 \|APBB1IP \|BLVRA \|MSL1 \|SCAF11 | 6.84426308 | 0.29852 | 0.659 | 0.23166 | −0.2221 | 0.931 |
| CX3CR1 \|RARS2 \|GSTM2 \|ATF6B \|DCP2 \|GK \|RBMS1 \|SNX5 \|F13A1 \|ACIN1 | 6.834418152 | 0.26614 | 0.93987 | 0.08427 | −0.8657 | 0.92569 |
| HIST2H2AA4 \|SF3B2 \|NPL \|LILRA6 \|ISCU \|LILRB1 \|POMT1 \|SMS \|WDR43 \|LILRA1 | 6.829797076 | 0.24379 | 0.74838 | 0.12137 | 0.73693 | 0.91662 |
| PSMC1 \|TP53 \|CCT3 \|LTBP3 \|TAZ \|XRN2 \|SART1 \|NUP88 \|MAPK1IP1L \|RAD21 | 6.822412078 | 0.29298 | 0.84458 | 0.2458 | −1.1286 | 0.93174 |
| IKZF1 \|PPP2CA \|WDR48 \|PPNRC2 \|RNPS1 \|TTC37 \|INIP1 \|GLB1 \|LRRC37B \|LBH | 6.814336427 | 0.26336 | 0.98052 | 0.05391 | −1.046 | 0.93059 |
| PYHIN1 \|SDCCAG8 \|PKN2 \|NUDT21 \|GART \|SMC1A \|SH3BP1 \|ROCK1 \|THOC2 \|EZR | 6.808609665 | 0.28476 | 0.77839 | 0.14257 | −0.9647 | 0.93246 |
| EHD1 \|SLA \|USP24 \|BNIP2 \|WDR11 \|GNS \|ST6GAL1 \|NDEL1 \|GLYR1 \|GYPC | 6.799798718 | 0.28059 | 0.83012 | 0.13164 | −0.2193 | 0.92611 |
| USP10 \|SKAP2 \|U2SURP \|PSMC3 \|IL11RA \|RGS18 \|PPP1CA \|RASGRP1 \|CSNK1G2 \|ARF1 | 6.788933731 | 0.27068 | 0.748 | 0.14031 | −0.7008 | 0.92902 |
| SNCA \|KTN1 \|SPCS2 \|SEMA4A \|RANBP3 \|SRSF6 \|ITM2A \|TAGAP \|MED12 \|CHD3 | 6.778873901 | 0.277 | 0.7476 | 0.12418 | −0.0312 | 0.92639 |
| NUFIP2 \|TOMM6 \|NDUFB4 \|CCNY \|APOBEC3A \|TYK2 \|CLCN7 \|NPLOC4 \|TMEM176B \|STARD7 | 6.769748193 | 0.27065 | 0.86438 | 0.15816 | 1.29761 | 0.9184 |
| TIAL1 \|PSMC6 \|ACAT1 \|PSMD8 \|CLN3 \|TAOK3 \|SERPINB6 \|SS18 \|ANKZF1 \|APOL3 | 6.765299374 | 0.26237 | 0.83055 | 0.20279 | −0.9252 | 0.92089 |
| HTT \|ATP5H \|CRTC3 \|ATP5O \|CD81 \|MTIF3 \|MAPRE2 \|PRPS1 \|GNPTAB \|TRAPPC11 | 6.758683093 | 0.28465 | 0.53589 | 0.30431 | −0.5062 | 0.92368 |
| LRCH3 \|GNB2 \|TOM1 \|PRKCD \|ATP2B1 \|AFF3 \|WWP2 \|OXA1L \|VDAC1 \|ACAD9 | 6.746939137 | 0.30374 | 0.93869 | 0.14778 | −0.9624 | 0.91756 |
| VPS378 \|PIP5K1A \|FBXW4 \|HOOK3 \|MSTO1 \|SRSF4 \|USP9Y \|RAB31 \|NCF4 \|FUNDC2 | 6.732544434 | 0.28252 | 0.90215 | 0.05547 | −0.6654 | 0.92906 |
| DDB1 \|MBD1 \|USP9X \|RUNX2 \|TUBA1C \|COTL1 \|TPM1 \|SNRNP27 \|CST7 \|TUBB4B | 6.722754156 | 0.26355 | 0.96256 | −0.0089 | −0.3187 | 0.92561 |
| PPIPSK2 \|PROSER1 \|ZNF506 \|MTCH1 \|RNF111 \|ANP32E \|FPR1 \|AKAP8L \|HGS \|KSR1 | 6.710437578 | 0.27723 | 0.82917 | 0.08726 | 0.31466 | 0.93539 |
| SCAF4 \|RC3H1 \|EIF4E3 \|PSMA4 \|UBR4 \|IRF2 \|PRPF31 \|EBXO11 \|SNX1 \|GPR89B | 6.700249789 | 0.29947 | 0.57048 | 0.28487 | 0.07038 | 0.9284 |
| GATA3 \|NPC1 \|RAP1A \|CHD9 \|MRPL33 \|ATIC \|HNRNPL \|CEP57 \|YWHAB \|LRPPRC | 6.692276714 | 0.28569 | 0.7037 | 0.15701 | −0.076 | 0.9251 |
| CTSB \|PSMA3 \|ATP5E \|LPCAT2 \|SCAMP2 \|TAF1 \|AKT3 \|LRRK2 \|SCIMP \|ATP5C1 | 6.684814512 | 0.26729 | 0.70763 | 0.24604 | −0.6516 | 0.92758 |
| STK38L \|TMBIM1 \|CD244 \|NADSYN1 \|RFC1 \|ZMYND11 \|R3HDM1 \|RAB11A \|ABR \|DHX9 | 6.675340745 | 0.28246 | 0.78126 | 0.18557 | −0.9949 | 0.92809 |
| CRTAP \|MDH2 \|TOP2B \|FIG4 \|KAT7 \|PITPNC1 \|ALOX5 \|GOLGA6L9 \|GNPTG \|CBLL1 | 6.66698328 | 0.30444 | 0.83997 | 0.20883 | −0.358 | 0.92113 |
| MVP \|IL10RB \|IUND \|DGKD \|ARID1B \|MANBA \|IL2RB \|DPYSL2 \|RBM10 \|ATP6V1A | 6.660737692 | 0.28155 | 0.6904 | 0.29432 | 1.60214 | 0.9139 |
| RAD23B \|CCR2 \|XAF1 \|DCAF8 \|GUSB \|IMPDH2 \|PDS5B \|HERC1 \|SBDS \|MAPK8IP3 | 6.655031306 | 0.27839 | 0.74842 | 0.15515 | −0.812 | 0.92864 |
| MAPK1 \|ATPIF1 \|AMPD2 \|DCTN1 \|MAP2K2 \|DPP7 \|CTSZ \|PRDX5 \|GSPT1 \|ATG7 | 6.646900323 | 0.29645 | 0.75029 | 0.18627 | −0.5944 | 0.9118 |
| CORO7 \|ARL8A \|NDUFA6 \|C8orf59 \|REPS1 \|TMEM33 \|AP3S1 \|NBEAL2 \|TRIP11 \|ARF5 | 6.642153115 | 0.30024 | 0.60894 | 0.2882 | −0.5761 | 0.92911 |
| PLXDC2 \|TTLL5 \|PITPNB \|CD28 \|EIF3G \|PARP15 \|TNFAIP8 \|MAPKAPK2 \|TMEM50A \|CAPN3 | 6.634381408 | 0.25343 | 1.04378 | 0.05614 | −0.6405 | 0.93677 |
| ZCCHC6 \|MAGOH \|PRMT1 \|HIST1H4H \|ECE1 \|FRYL \|NSFL1C \|TNRC6B \|YME1L1 \|TPR | 6.628466444 | 0.30891 | 0.68872 | 0.20758 | −0.8057 | 0.93178 |
| NDUFA12 \|MYO1G \|RABEP1 \|BSPB1 \|POLR3E \|WDR60 \|PRPF6 \|DLG1 \|CBFB \|PLSCR1 | 6.621769217 | 0.2751 | 0.79958 | 0.21681 | −0.968 | 0.92575 |
| GOLGA8B \|ARHGAP30 \|CHP1 \|RNF220 \|RPN1 \|SYF2 \|RARRES3 \|CD2BP2 \|TMEM2 \|SNX3 | 6.614640949 | 0.27796 | 0.81948 | 0.16873 | −1.0554 | 0.91162 |
| EID1 \|TRAPPC9 \|WWOX \|UBE2G2 \|ADSL \|XPO7 \|UBE2Q1 \|ARMC8 \|POM121 \|ACADM | 6.603934806 | 0.27899 | 0.91665 | 0.14736 | −0.3037 | 0.93199 |
| TSPYL2 \|UTY \|TSC2 \|UBXN1 \|MEFV \|RBM28 \|PTPRE \|TMX2 \|TYMP \|CEP170 | 6.592683679 | 0.2843 | 0.96725 | 0.15061 | 0.89598 | 0.91837 |
| HELZ \|ILF2 \|SYNGR2 \|ZNF721 \|SMC3 \|NR4A2 \|R3HDM2 \|SAP18 \|RALGDS \|MTA2 | 6.584757143 | 0.31657 | 0.61839 | 0.27841 | −0.9307 | 0.92096 |
| PHF20L1 \|VPS4B \|CEP104 \|KDM2A \|SKIV2L2 \|BRD4 \|ATP13A3 \|BRWD3 \|HINT1 \|RBM26 | 6.575823315 | 0.26939 | 0.93387 | 0.07389 | −0.6462 | 0.9392 |
| SACM1L \|SIPA1 \|NR2C2 \|SREBF2 \|INPPL1 \|CD226 \|TTC13 \|C12orf57 \|TTC17 \|MFSD6 | 6.568123832 | 0.2935 | 0.88516 | 0.03728 | −1.0754 | 0.92578 |
| FOSL2 \|STK38 \|CD8A \|ITPR2 \|OCIAD1 \|DAD1 \|UBE2V1 \|CHMP2A \|ATP5A1 \|CCZ1 | 6.561238162 | 0.28809 | 0.69832 | 0.15203 | −0.9746 | 0.93063 |
| PITPNA \|ASXL2 \|PSMD7 \|ZFC3H1 \|SIL1 \|CCDC88A \|CHMP3 \|MBD4 \|PEPD \|KIAA1468 | 6.554029 | 0.29915 | 0.69055 | 0.17462 | 0.03045 | 0.93078 |
| SRP72 \|ARPC4 \|AGPS \|TAF2 \|CTDSP2 \|NFAM1 \|KANSL1 \|SLX1A \|CNN2 \|COL6A2 | 6.548570068 | 0.28027 | 1.06451 | 0.0894 | −0.9577 | 0.90581 |
| NLRP1 \|MKLN1 \|YPEL5 \|COPB2 \|UBA3 \|PPP3R1 \|MOB1A \|OMA1 \|EPC1 \|LRRC37A3 | 6.536208167 | 0.29465 | 0.76997 | 0.2066 | −1.0509 | 0.93465 |
| DYNC1I2 \|CYCS \|PCNT \|CABIN1 \|KJAA0100 \|WDR7 \|DENND5A \|ADK \|CPD \|PPWD1 | 6.526136762 | 0.28234 | 0.6522 | 0.17125 | −0.508 | 0.93866 |
| NFYC \|PCBP1 \|CEPT1 \|FERMT3 \|SPAST \|ZFY \|HIST1H3A \|ARIH1 \|DHX15 \|NAIP | 6.511695733 | 0.30927 | 0.84734 | 0.11615 | −1.018 | 0.93374 |
| SEC61A1 \|APPL2 \|ERLIN2 \|ZMYM4 \|SH3GLB1 \|RCSD1 \|TLR4 \|LRCH4 \|LDB1 \|RAC1 | 6.504130054 | 0.29562 | 0.70975 | 0.1174 | 0.25141 | 0.92877 |
| ARHGEF6 \|VPS39 \|SLC20A1 \|TGFBR1 \|ELP3 \|IVD \|GATAD2B \|PTDSS1 \|NDUFS4 \|TCEA1 | 6.498779904 | 0.28078 | 0.71864 | 0.19316 | −0.1422 | 0.94336 |
| MECP2 \|ABI1 \|DNAJA1 \|MLLT6 \|IKEKB \|BST1 \|SMARCA4 \|ARFGAP2 \|IRF2BP2 \|WDR59 | 6.493194244 | 0.30493 | 0.64869 | 0.22332 | −0.5844 | 0.92038 |
| KRI1 \|LRMP \|UBE2F \|SPARC \|COX5B \|GNPAT \|SCO2 \|CCDC159 \|ZNF708 \|TNFRSF1B | 6.482911392 | 0.28563 | 0.96765 | 0.08657 | −0.1991 | 0.92326 |
| MGST2 \|CEP350 \|CD86 \|RGS10 \|OFD1 \|NPAT \|TLK2 \|FOXN3 \|DNAJB14 \|EIF4E | 6.475045674 | 0.27634 | 0.77871 | 0.1509 | −0.6427 | 0.93331 |
| TESPA1 \|PRKAA1 \|KAT6B \|CES1 \|WDR70 \|ATP9B \|USP32 \|PGAM1 \|ANKRD10 \|SPECC1L | 6.468326366 | 0.27744 | 0.76016 | 0.09989 | −0.5745 | 0.93518 |
| TSEN54 \|SUPT5H \|INFAIP3 \|ZNF706 \|CHD1 \|TM9SF2 \|SPN \|CD47 \|MAML2 \|ANKH | 6.462408418 | 0.26196 | 0.85331 | 0.12921 | 0.21474 | 0.92792 |
| ARRDC1 \|CHFR \|PLRG1 \|LMBRD1 \|GOLGA4 \|IFNGR1 \|IGF2R \|NASP \|VEZF1 \|MTCH2 | 6.454520819 | 0.29824 | 0.65368 | 0.3012 | −0.9696 | 0.93364 |
| NUDC \|AUP1 \|ZNF431 \|URI1 \|TCF7L2 \|ARNT \|MAP2K1 \|ISG20 \|DDX47 \|NADK | 6.44626702 | 0.27898 | 0.64693 | 0.23479 | −0.9208 | 0.92927 |
| SURF4 \|COX8A \|PLCG2 \|FAM102A \|HARS2 \|RGS14 \|RAPGEF6 \|PPFIA1 \|DAZAP1 \|RHOC | 6.440008642 | 0.2919 | 0.67404 | 0.26118 | −0.0811 | 0.91292 |
| OSBPL2 \|DDX46 \|IRAK3 \|TNFSF13 \|MYO18A \|PRKACB \|METAP2 \|CTBP1 \|SSFA2 \|ATF2 | 6.433822171 | 0.28369 | 0.82116 | 0.10817 | −0.6641 | 0.92683 |
| GCLC \|BCAS3 \|IDH2 \|HDAC7 \|VAMP3 \|IRF3 \|POLR3GL \|CNPY3 \|LSM6 \|RNF167 | 6.426133188 | 0.29749 | 0.59983 | 0.2653 | 0.34464 | 0.93242 |
| GPATCH8 \|VAV1 \|TRIO \|MPV17 \|ZDHHC20 \|BIN1 \|GSK3B \|HDAC3 \|FRY \|DCLRE1C | 6.414966746 | 0.27985 | 0.72961 | 0.16962 | 1.0294 | 0.92443 |
| RASA3 \|CDC73 \|SERINC3 \|CCDC91 \|PKN1 \|LRRFIP2 \|USP34 \|SERF1B \|INPP5K \|CDC27 | 6.407689693 | 0.30014 | 0.84689 | 0.18743 | −0.6506 | 0.93261 |
| ZNF280D \|SPECC1 \|CD79A \|ZDHHC3 \|SCNM1 \|IDS \|GANAB \|RAPGEF1 \|ZNF561 \|SARNP | 6.402637039 | 0.25335 | 0.8352 | 0.13787 | −0.3128 | 0.92248 |
| EMD \|GPBP1 \|EIF1AY \|PARL \|PRDX1 \|LNPEP \|AAMP \|EZH1 \|PRAM1 \|SEL1L3 | 6.398390047 | 0.26559 | 0.76317 | 0.13729 | −0.7347 | 0.92645 |
| CNTRL \|LASP1 \|CIZ1 \|WDR26 \|PBRM1 \|PMS1 \|INPP4B \|MX2 \|NF1 \|BIST1H28B | 6.391064253 | 0.28282 | 1.07643 | −0.0152 | 0.24804 | 0.92235 |
| FAM133B \|HSD17B11 \|PCF11 \|TBC1D1 \|COPZ1 \|ZNF780B \|ZZEF1 \|GTF2H2C \|CDK12 \|SNX14 | 6.38355414 | 0.28852 | 0.77925 | 0.12047 | −0.9274 | 0.93507 |
| GTF2A1 \|ESYT2 \|DIS3L2 \|DDOST \|NCOA2 \|PSMB10 \|GBA2 \|RALB \|SHMT2 \|PSME3 | 6.377543559 | 0.29292 | 0.92221 | 0.1466 | −0.79 | 0.92179 |
| ACSF3 \|RBM27 \|ARNTL \|HECTD1 \|KYNU \|WBP2 \|NLRP3 \|COQ9 \|TNRC18 \|TMBIM4 | 6.370811114 | 0.28 | 0.95713 | 0.11247 | −0.29 | 0.93584 |
| CELF1 \|AGFG1 \|XPC \|BRAF \|UBQLN1 \|SSB \|SOAT1 \|APRT \|ANXA4 \|NSA2 | 6.364987807 | 0.27226 | 0.91075 | 0.08577 | 0.67996 | 0.93954 |
| KXD1 \|LCMT1 \|DNAJC7 \|CHMP7 \|LPAR6 \|TTC21B \|HSPA4 \|CPSF6 \|MAP3K4 \|EKBP15 | 6.359258166 | 0.27309 | 0.83694 | 0.11404 | −0.0348 | 0.94512 |
| MED23 \|RUNX3 \|SMG7 \|APAF1 \|RAB21 \|ATXN7L1 \|PDK2 \|RNF38 \|NKG7 \|PPFIBP2 | 6.350084371 | 0.25207 | 0.80838 | 0.11436 | 0.32481 | 0.91559 |
| DYNLRB1 \|HBS1L \|ZEB1 \|PYGL \|PRPF38B \|USP36 \|FLOT2 \|OST4 \|CRLF3 \|CLCN3 | 6.345684413 | 0.29606 | 0.66798 | 0.27635 | −0.5933 | 0.92705 |
| GCC2 \|MPHOSPH8 \|LPIN2 \|FCHSD2 \|AGA \|ZCCHC7 \|SRSF7 \|RASA2 \|FBL \|DBX38 | 6.337378701 | 0.28981 | 0.85543 | 0.14236 | 0.79858 | 0.93489 |
| GANC \|NUP98 \|CDS2 \|MTMR3 \|FBXL5 \|CAPG \|ACPP \|CCDC59 \|RIOK3 \|OGDH | 6.327793224 | 0.29229 | 0.76081 | 0.04965 | −0.5859 | 0.92886 |
| GNG2 \|MAT2A \|PDXDC1 \|DMXL1 \|C11orf57 \|DDX19A \|PHC1 \|RBP7 \|API5 \|ZFX | 6.323731183 | 0.27599 | 0.8375 | 0.13401 | −0.2289 | 0.93299 |
| MAT2B \|RBM12 \|CA2 \|PMPCB \|PAF1 \|CAMK2D \|MRPS18C \|TRAF5 \|MPHOSPH9 \|RTN4 | 6.316616626 | 0.27689 | 0.77919 | 0.25124 | −0.3105 | 0.93321 |
| ABCB7 \|RASSF3 \|ZMIZ2 \|ATP6V1G1 \|ARL6IP4 \|NISCH \|C6orf89 \|AAAS \|SPDYE1 \|CLASP2 | 6.311733309 | 0.29023 | 0.72865 | 0.18261 | −0.9535 | 0.92536 |
| ZNF330 \|SPAG9 \|CERS2 \|CD300LF \|OSBPL9 \|VTI1A \|ATG4B \|PUM1 \|HIST1H2AI \|PSMC2 | 6.306984974 | 0.27713 | 0.84018 | 0.15224 | 0.02481 | 0.93834 |
| MAP3K3 \|YTHDF3 \|STARD3 \|ATP6V0E1 \|ZBTB8OS \|PPP1R10 \|TTN \|TNIK \|ARAP2 \|GFM1 | 6.302998962 | 0.28148 | 0.94965 | 0.16864 | −0.3894 | 0.93459 |
| PRPF38A \|PDCD6 \|GPD2 \|POLR1D \|ECHDC2 \|CDK4 \|DMXL2 \|NCK1 \|DLST \|MLXIP | 6.29613495 | 0.28109 | 0.75311 | 0.21873 | −0.3749 | 0.92572 |
| NDUFA4 \|AGPAT3 \|PMS2 \|FNDC3A \|DENND4A \|TAGLN \|AGTPBP1 \|ZFYVE26 \|PPOX \|POLG | 6.290030039 | 0.26938 | 0.76769 | 0.13456 | −0.2475 | 0.92596 |
| LEPROTL1 \|CLASP1 \|COX7B \|GPBP1L1 \|TBC1D4 \|GHITM \|TBC1D14 \|SPOP \|PSMB1 \|MDFIC | 6.285699439 | 0.28881 | 0.77782 | 0.26876 | −0.8921 | 0.93391 |
| CHN2 \|SENP2 \|HIF1AN \|KIDINS220 \|NEK7 \|CEP63 \|MEF2A \|RNASE2 \|STX8 \|USO1 | 6.278683944 | 0.27955 | 0.83488 | 0.16001 | 0.93237 | 0.93688 |
| DDX21 \|EIF2S1 \|PSMD13 \|BTAF1 \|HISTIH2AH \|FMR1 \|CHORDC1 \|EIF3I \|PSD4 \|SARS | 6.272754415 | 0.29606 | 0.78375 | 0.25683 | −0.8622 | 0.93714 |
| ALOX5AP \|CLTA \|CSTB \|UBXN7 \|GRIPAP1 \|RFX5 \|FOXJ3 \|RNF31 \|AKR1A1 \|UHRF2 | 6.268015972 | 0.292 | 0.67795 | 0.26303 | −0.6006 | 0.93006 |
| PMM2 \|WDR33 \|MTG1 \|NCOR2 \|SIGLEC14 \|DYRK1A \|UBE4A \|CD14 \|FAR1 \|HNMT | 6.260391626 | 0.25205 | 0.84051 | 0.14646 | −0.5742 | 0.93866 |
| MTHED1 \|UBR5 \|ARHGEF3 \|GALNS \|FYTTD1 \|ARHGAP17 \|UBAC2 \|TMEM214 \|COG1 \|VPS33B | 6.254243189 | 0.28175 | 0.63329 | 0.3324 | −0.0518 | 0.92236 |
| CERK \|CDK13 \|RAB11FIP4 \|NDUFV3 \|PTGES3 \|OSTM1 \|UBE2H \|LAP3 \|BCL7B \|HIPK1 | 6.249613549 | 0.28499 | 0.43697 | 0.33966 | −0.4185 | 0.91007 |
| RP86K81 \|ZNF143 \|XRRA1 \|GRK6 \|ATF7 \|TBP \|PABPC4 \|CCDC82 \|STT3B \|DEPDC5 | 6.245426336 | 0.29899 | 0.8015 | 0.09216 | −0.5614 | 0.94427 |
| PDXK \|APEH \|NRBP1 \|GATAD2A \|RAB5B \|INO80 \|POLDIP3 \|STIM2 \|AP2B1 \|TFAM | 6.240370576 | 0.29864 | 0.73091 | 0.1139 | −0.8996 | 0.92344 |
| OGG1 \|MCCC2 \|ATP8B1 \|ZNF33A \|TSN \|STRN \|RBBP7 \|LRP1 \|BLOC1S1 \|ANO6 | 6.232822641 | 0.2725 | 0.65902 | 0.22554 | −0.696 | 0.9252 |
| ENTA \|WBP11 \|PNPLA2 \|LAMP2 \|STRN3 \|STT3A \|USF1 \|AP1M1 \|BUB3 \|DDHD2 | 6.227018167 | 0.29082 | 0.77441 | 0.21613 | −0.8072 | 0.92747 |
| TNFSF13B \|ASH2L \|ENY2 \|DHRS7 \|INO80E \|NDUFAF5 \|TIMM23 \|NPTN \|TK2 \|PPP1CC | 6.218269306 | 0.27184 | 0.95444 | 0.0962 | −0.7978 | 0.93509 |
| SRPK2 \|DDX18 \|HNRNPF \|OAS2 \|C9orf72 \|BTN2A2 \|NOLC1 \|CD5 \|COPG1 \|PPHLN1 | 6.210848813 | 0.25983 | 0.82138 | 0.27726 | −0.7353 | 0.92886 |
| SPTBN1 \|NSRP1 \|PHE8 \|PEBP1 \|OLA1 \|GYS1 \|ZNF512 \|STOM \|BIRC2 \|C20orf24 | 6.203761744 | 0.29023 | 0.74258 | 0.19251 | 0.18781 | 0.92548 |
| BAG6 \|TIMP1 \|TNPO1 \|AIP \|MKNK2 \|PSMD2 \|RNF125 \|CFDP1 \|PHF3 \|CD163 | 6.199715897 | 0.29173 | 0.88669 | 0.07759 | −0.4668 | 0.93343 |
| VPS52 \|CCNT2 \|DRAP1 \|PIK3C3 \|BK3 \|PSMC5 \|CLNS1A \|NCOA7 \|APOBEC3G \|PWWP2A | 6.195172192 | 0.29028 | 0.73228 | 0.15249 | −1.0295 | 0.93427 |
| UBXN11 \|HDAC9 \|HIST1H4D \|MAN2A2 \|KRR1 \|NCOA3 \|VPS13D \|VPS13A \|PTPN2 \|MBD2 | 6.189389513 | 0.27415 | 1.03896 | 0.08439 | −0.6941 | 0.93793 |
| SRRT \|FAS \|ARFGEF2 \|ATP6V0D1 \|SRSF10 \|RNF138 \|CHMP4A \|LONP2 \|APPL1 \|ITFG1 | 6.185411363 | 0.29913 | 0.83113 | 0.12562 | −0.9142 | 0.93127 |
| NT5DC1 \|ZFP36L2 \|PIKFYVE \|DYNLT1 \|MED28 \|CCT8 \|AFF4 \|CIR1 \|RFXANK \|ZNF430 | 6.180798559 | 0.2997 | 0.7424 | 0.28755 | −0.6699 | 0.9415 |
| ZDHHC17 \|METTL17 \|PHF11 \|SECISBP2L \|PARG \|DCTN2 \|ANAPC5 \|MBTPS1 \|LMBR1L \|AKR1B1 | 6.173620149 | 0.27947 | 0.73367 | 0.24973 | −0.3474 | 0.9382 |
| TMEM183A \|MPP7 \|MEMO1 \|RICTOR \|ZMAT2 \|ZNF609 \|IREB2 \|EIF2B2 \|EIF3B \|BAP1 | 6.166807102 | 0.26466 | 0.72885 | 0.13915 | −0.6786 | 0.93704 |
| PIEZO1 \|EFR3A \|PPP1R12C \|WDR45 \|KIAA0319L \|SEC61B \|ZNF737 \|MRPS5 \|CCNH \|VAV3 | 6.161641235 | 0.29689 | 0.86709 | 0.19492 | −0.4618 | 0.93433 |
| RALGPS2 \|ETFB \|RORA \|IPO8 \|TMED2 \|ATXN2L \|TNFRSF10A \|FDFT1 \|SLC25A28 \|ORC4 | 6.155370501 | 0.2647 | 0.78861 | 0.1849 | −0.4696 | 0.92664 |
| G6PC3 \|ZNF800 \|ZNF253 \|PSENEN \|GAS7 \|DDX60L \|ERP29 \|BECN1 \|SNF8 \|CLPTM1L | 6.149531148 | 0.28496 | 0.79373 | 0.10144 | −0.1926 | 0.92343 |
| UTP6 \|TGFBR2 \|SEC23IP \|CCDC7 \|YARS \|ACOX1 \|FAM117A \|VRK1 \|S100A10 \|SPPL3 | 6.144997199 | 0.28551 | 0.8626 | 0.14949 | −0.3715 | 0.93007 |
| AP1G1 \|CSNK2B \|CDK11B \|PDK1 \|STAT4 \|CUX1 \|NFE2L2 \|CD84 \|SFI1 \|INPP4A | 6.140040609 | 0.2831 | 0.85966 | 0.13154 | −0.5701 | 0.94011 |
| SUPT6H \|MLLT10 \|ATP2B4 \|ZDHHC7 \|RAB3GAP1 \|DYNC1LI2 \|ATP11A \|RFFL \|SETD2 \|DUS3L | 6.131737275 | 0.29841 | 0.66845 | 0.16922 | −0.6543 | 0.9248 |
| NGLY1 \|MAP4K5 \|FAM193B \|DROSHA \|SIGIRR \|GAB3 \|VPS29 \|PFKL \|POLR2E \|OGFR | 6.122622564 | 0.28369 | 0.70503 | 0.15701 | −0.8874 | 0.91929 |
| KAT2B \|NUP54 \|IFRD1 \|ZFAND6 \|ZMYM2 \|APC \|ABCC1 \|CDC123 \|C5orf56 \|MAGT1 | 6.118571537 | 0.29129 | 0.5858 | 0.25829 | −0.4365 | 0.92869 |
| TTC7A \|D1ABLO \|MAP7D1 \|NOL8 \|MDB1 \|SNW1 \|VNN2 \|EGR1 \|RNF4 \|PIGT | 6.115075422 | 0.27479 | 0.98354 | 0.19785 | −0.7309 | 0.93214 |
| RGPD1 \|ZNF267 \|YWHAH \|C6orf106 \|ANAPC1 \|SNRPA1 \|UGP2 \|ACP1 \|ATP6V0A2 \|CUTC | 6.108195742 | 0.287 | 0.52228 | 0.24898 | −1.0678 | 0.92408 |
| SHC1 \|PHKA2 \|NBN \|GPX1 \|SERPINF1 \|C14orf159 \|ERCC5 \|PI4KB \|TTLL4 \|ABCE1 | 6.101609752 | 0.26977 | 1.04482 | 0.0534 | −0.6274 | 0.92585 |
| MICAL2 \|PARP14 \|TRIM25 \|WDR37 \|AHCYL1 \|APBA2 \|CCNL2 \|KIAA1143 \|CLK2 \|TTC19 | 6.092012631 | 0.28693 | 0.71727 | 0.14411 | 0.23218 | 0.91984 |
| BRWD1 \|SREBF1 \|ADPGK \|TPCN1 \|ZDHHC6 \|SEC13 \|PARN \|RYK \|DNASE1 \|MAPK14 | 6.083774987 | 0.2869 | 0.77745 | 0.23913 | −0.914 | 0.92251 |
| ZNF587 \|SRPK1 \|FDPS \|HMGA1 \|COG6 \|FEZ2 \|TTBK2 \|MTMR1 \|TRAF3 \|IPO5 | 6.077609251 | 0.26723 | 0.92803 | 0.09661 | −0.8216 | 0.93745 |
| CAMK4 \|KIAA0368 \|BLNK \|FIS1 \|REV3L \|BDP1 \|SETDB1 \|ERAP2 \|AMFR \|EXOC1 | 6.071411817 | 0.26005 | 1.00869 | 0.06535 | −0.8163 | 0.94202 |
| PARP4 \|ZNF592 \|SAR1B \|YY1 \|TRIM52 \|SURF1 \|ANKIB1 \|NOTCH2NL \|SNX19 \|LCK | 6.065807251 | 0.24271 | 0.82544 | 0.11148 | −0.5033 | 0.9375 |
| SNRPD3 \|PMPCA \|PPP3CA \|MON2 \|FAM126A \|ITFG2 \|SBF2 \|UBE2K \|TXN \|MAP3K2 | 6.06076855 | 0.29354 | 0.70488 | 0.21225 | 0.2972 | 0.92505 |
| PRRC1 \|ABCF3 \|WTAP \|ARF3 \|KIR2DL1 \|RABL2A \|PPIL2 \|SUCLG1 \|PDE4DIP \|SHISA5 | 6.054930389 | 0.23507 | 1.11255 | 0.08046 | −0.1733 | 0.92667 |
| PTPRJ \|GRAP \|NCBP2 \|MPP1 \|PPP3CB \|RHOT1 \|UROD \|TAPBP \|PDE7A \|CCNDBP1 | 6.047671052 | 0.2956 | 0.81998 | 0.22384 | −0.8721 | 0.92268 |
| ITPK1 \|RNF34 \|CTDNEP1 \|PCMTD2 \|UBA6 \|FCGRT \|TUBB \|SAMD9 \|TARS \|CTPS1 | 6.04088478 | 0.29395 | 0.71363 | 0.21561 | −0.6456 | 0.93083 |
| CNOT8 \|SNRK \|BANF1 \|CLSTN3 \|NDUFAF2 \|BAG4 \|SLEN5 \|UBAP2 \|PSMB7 \|DDB2 | 6.033176193 | 0.29197 | 0.78746 | 0.17163 | −0.5425 | 0.92769 |
| UBE2N \|PARP12 \|ATP13A1 \|PTCH1 \|LARP7 \|MKNK1 \|PSPC1 \|SRP9 \|SNRPN \|GOLGA7 | 6.023580906 | 0.28977 | 0.91834 | 0.04448 | −0.0801 | 0.94261 |
| JAZF1 \|HTATIP2 \|PREX1 \|NBAS \|PHF12 \|FCF1 \|HMG20A \|IFI44 \|NDUFS8 \|SFT2D2 | 6.017099086 | 0.27379 | 0.82672 | 0.31107 | −0.5466 | 0.92936 |
| SIGLEC10 \|ARID2 \|PLXNC1 \|HMBOX1 \|TBRG1 \|UBE2E3 \|MFSD8 \|UBXN2B \|ANKRD13D \|POLK | 6.011237751 | 0.27311 | 0.81063 | 0.11253 | −0.0359 | 0.92555 |
| ASCC1 \|KHSRP \|STAU2 \|SP2 \|RTN3 \|VDAC2 \|IARS \|SCML4 \|TDP1 \|GGA3 | 6.006014062 | 0.2947 | 0.56507 | 0.20156 | 0.57437 | 0.93488 |
| FBXW2 \|MIS18BP1 \|FAM49A \|FAF1 \|DHX36 \|PAN3 \|LEMD2 \|EIF2AK2 \|ACTR10 \|HMOX1 | 5.999566491 | 0.29147 | 0.6104 | 0.27618 | −0.4502 | 0.93333 |
| AP2A2 \|ARL6IP1 \|TXNRD1 \|UPF3A \|NAA60 \|UBE2Q2 \|SLC35E2 \|SAMD3 \|PDPR \|TNFSF10 | 5.995155126 | 0.25854 | 0.92575 | 0.11814 | −0.3909 | 0.9279 |
| TNERSF10B \|MYO1E \|ZC3H13 \|RSPRY1 \|BLK \|L3MBTL3 \|NUCB2 \|LYPLA1 \|PIM2 \|OSTC | 5.990122237 | 0.25669 | 0.92289 | 0.09298 | −0.6445 | 0.94216 |
| DCAF5 \|CCNK \|C2CD3 \|ARHGAP1 \|NDUFB2 \|SLBP \|MAPK3 \|MCTP1 \|MRPS25 \|SDAD1 | 5.982237204 | 0.29695 | 0.66209 | 0.26443 | −0.4621 | 0.92196 |
| USP33 \|DRG1 \|AEBP2 \|SPPL2A \|NDST2 \|MSL2 \|GRAPL \|DCAF11 \|KLF2 \|ARHGEF7 | 5.974321476 | 0.29846 | 0.73123 | 0.15446 | −0.8079 | 0.92341 |
| POGZ \|DCTN4 \|VPS53 \|GAK \|DENR \|PRCC \|CDK9 \|BACH1 \|PPA1 \|FLT3LG | 5.971180975 | 0.29965 | 0.60677 | 0.30139 | −0.9108 | 0.92252 |
| PLD3 \|ICAM3 \|ALDH3A2 \|NAPA \|CCPG1 \|UROS \|MLX \|BRD9 \|AHR \|RAB11FIP1 | 5.965511172 | 0.2871 | 0.77596 | 0.08558 | −0.4737 | 0.92431 |
| RABGAP1 \|DDT \|ZCCHC2 \|HDHD2 \|FAM204A \|LIG3 \|PLEKHO1 \|MAP3K8 \|8NX6 \|DDX19B | 5.957375238 | 0.28762 | 0.86855 | 0.24924 | −0.2033 | 0.9337 |
| STRN4 \|ASH1L \|ADCY7 \|NDUFB7 \|MYADM \|MPHOSPH6 \|G6PD \|PRDM2 \|DOCK9 \|DNAH1 | 5.953109704 | 0.23447 | 1.14453 | 0.06448 | −0.9563 | 0.91923 |
| RNF2 \|PCSK7 \|AIFM1 \|ELOVL1 \|PKD1 \|PSMA5 \|SNX13 \|BOD1L1 \|PLAUR \|TMEM248 | 5.947548875 | 0.29886 | 0.68648 | 0.21672 | −0.3588 | 0.93097 |
| SART3 \|AP162 \|TXNDC15 \|KHDRBS1 \|SYVN1 \|ACOT9 \|INTS10 \|SRP54 \|MGAT4B \|METTL22 | 5.941018497 | 0.27067 | 0.98714 | 0.08035 | −0.8891 | 0.92782 |
| CYFIP1 \|CD33 \|THOC7 \|ITGB1BP1 \|FCGR2B \|ACAA1 \|TRABD2A \|P2RY10 \|WDFY3 \|ANAPC15 | 5.935025345 | 0.27036 | 0.81771 | 0.23531 | 0.64531 | 0.9178 |
| EHBP1L1 \|PAFAH1B2 \|TMEM87A \|AATF \|GZMK \|ELMO2 \|COG4 \|PNP \|MAP2KS \|CRTC2 | 5.928213629 | 0.28063 | 0.80588 | 0.19413 | −0.8404 | 0.93445 |
| SESTD1 \|ARHGAP24 \|CRYZL1 \|SH3BGRL3 \|EIF4H \|FAM208A \|ECH1 \|DDH3G \|USP28 \|ACTN1 | 5.922097266 | 0.29103 | 0.73204 | 0.15861 | −0.8827 | 0.92676 |
| ATG13 \|CASP10 \|IPCEF1 \|PTTG1IP \|EIF4ENIF1 \|ZDHHC2 \|RIOK2 \|DCP1A \|GRHPR \|GNL3 | 5.917877968 | 0.27416 | 0.891 | 0.13321 | −0.0579 | 0.94038 |
| VPS36 \|DIDO1 \|GABARAPL1 \|SLC3A2 \|MRPL20 \|TCP1 \|CENPT \|DERL1 \|OXR1 \|TMED5 | 5.914156406 | 0.28547 | 0.80483 | 0.16239 | −0.8571 | 0.9361 |
| ADAM28 \|ADAM9 \|GSTO1 \|WDR36 \|XRCC3 \|CSAD \|CALCOCO1 \|ADA \|EPBX1 \|DNAJC21 | 5.908966186 | 0.24575 | 0.90648 | 0.1287 | −0.8189 | 0.9334 |
| ZNF276 \|TEX264 \|LUC7L \|NOP2 \|ABCA2 \|MLH3 \|WIPI1 \|METTL14 \|IKKEKG \|CC2D1B | 5.904011795 | 0.2942 | 0.68712 | 0.22565 | −0.9751 | 0.92044 |
| TMEM167A \|DNAJC13 \|TBCE \|CY85R3 \|RAB37 \|CCDC47 \|HEATR5B \|MYBL1 \|ABBD2 \|POMP | 5.895583312 | 0.27987 | 0.84498 | 0.16871 | −0.9749 | 0.93142 |
| SNX17 \|INTS3 \|DEF6 \|BMI1 \|PIH1D1 \|ZNF394 \|VPS26A \|BBS4 \|GABARAPL2 \|ACSL5 | 5.885258644 | 0.29362 | 0.8738 | 0.22466 | 0.34337 | 0.92069 |
| CLPX \|POU2F2 \|RC3H2 \|XPA \|NR2C1 \|SASH3 \|MMS19 \|SRSF1 \|PRMT5 \|HERPUD2 | 5.881162854 | 0.27568 | 1.00221 | 0.11207 | −0.6828 | 0.93267 |
| CNOT7 \|RBM34 \|ZNF468 \|NUP93 \|PPM1A \|NBR1 \|PIK3CB \|RAD23A \|SSBP1 \|STXBP3 | 5.876806878 | 0.28198 | 0.8821 | 0.08845 | −0.7351 | 0.9395 |
| ABCB1 \|EDEM1 \|TERF2 \|USP39 \|PSMD1 \|DPF2 \|NDUFC2 \|NUDT5 \|MICAL1 \|RASSF4 | 5.873723256 | 0.27984 | 0.90183 | 0.08996 | −0.4193 | 0.93865 |
| KLRF1 \|SLX1B \|SYNGAP1 \|RFC4 \|CAPN7 \|SCLT1 \|PDAP1 \|GABBR1 \|MGA \|CBWD6 | 5.869845846 | 0.27584 | 0.81804 | 0.12917 | −0.6119 | 0.94817 |
| MUTYH \|PRCP \|ARCN1 \|NF2 \|BRI3 \|UBXN4 \|KDM3A \|SIGLEC9 \|ATP6V0A1 \|IL18RAP | 5.866559142 | 0.28653 | 0.83176 | 0.22121 | 0.2791 | 0.93419 |
| RMND5A \|PHF1 \|SEC24B \|NCR1 \|ATPAF2 \|NDUFB10 \|OBSCN \|GUK1 \|STX12 \|RNF181 | 5.862069847 | 0.27236 | 0.678 | 0.15247 | −0.7565 | 0.90646 |
| TMOD3 \|PLEKHM2 \|ZNF595 \|OAS1 \|EAPP \|BLA-DMB \|SIPA1L1 \|HVCN1 \|SLAIN1 \|MORC3 | 5.857111482 | 0.23575 | 1.14711 | 0.05366 | 0.44146 | 0.92833 |
| ANPEP \|ARAF \|PTPN18 \|ECD \|MIM1 \|PNKD \|DARS \|NDUFA2 \|ATP6V1E1 \|OSGEP | 5.851031107 | 0.29713 | 0.60588 | 0.30858 | −0.5938 | 0.92879 |
| NAA10 \|MRPS27 \|IFT80 \|VPS28 \|TECPR1 \|JKAMP \|CALU \|COPB1 \|RAD50 \|EIF4G3 | 5.842788877 | 0.29697 | 0.6984 | 0.30109 | −0.7736 | 0.93421 |
| CD40 \|DDX11 \|ME2 \|PLOD3 \|MYSM1 \|CUL4B \|RNF40 \|LAT \|BFAR \|TREX1 | 5.839600405 | 0.27313 | 0.90067 | 0.08019 | −0.6696 | 0.91244 |
| CDC40 \|AP3S2 \|PQLC3 \|MED14 \|OXNAD1 \|KPNA6 \|CTDSPL2 \|BCL2L2-PABPN1 \|AP4B1 \|PLXNB2 | 5.833931663 | 0.28959 | 0.57759 | 0.1202 | −0.4754 | 0.92392 |
| MINK1 \|SSBP2 \|PHKG2 \|SCYL2 \|AKT1 \|DHRS4 \|PAK2 \|SMARCAD1 \|DNAJB9 \|ZNF778 | 5.827513274 | 0.29963 | 0.80361 | 0.1471 | −0.0002 | 0.92768 |
| C1QBP \|SMARCAL1 \|CPSF2 \|GNA13 \|LMBR1 \|MSH6 \|NAT10 \|LARP4 \|GTF3C2 \|PPM1M | 5.819024559 | 0.29639 | 0.63932 | 0.25544 | −0.9922 | 0.92555 |
| TTLL3 \|F5 \|THUMPD3 \|RFX3 \|RGPD2 \|KLHDC10 \|DSCR3 \|S1PR1 \|ABHD12 \|HMGB2 | 5.811195158 | 0.28195 | 0.86564 | 0.14792 | −0.7811 | 0.93486 |
| PSMD6 \|ANKMY1 \|LTBR \|RBM38 \|ZC3H4 \|FIBP \|RHOF \|CTR9 \|NLRP12 \|PXK | 5.806681988 | 0.29238 | 0.90068 | 0.20396 | −0.2413 | 0.91408 |
| GALNT1 \|EPRS \|SMC5 \|AKTIP \|ERCC6 \|KDELR2 \|PCMTD1 \|ADIPOR1 \|GNG5 \|VRK3 | 5.801849043 | 0.2827 | 0.70894 | 0.24232 | −0.4361 | 0.93462 |
| SCAPER \|RAB5A \|REL \|ABCD3 \|CMIP \|PDHB \|EXOC5 \|LPBA \|NFKB1 \|EMC2 | 5.796496264 | 0.30061 | 0.63806 | 0.18385 | 0.09791 | 0.93333 |
| NDUFB8 \|TMEM135 \|MT1X \|HIST1H3F \|WWP1 \|SPOPL \|STX10 \|NAA16 \|AQR \|VP837A | 5.792962775 | 0.28231 | 0.76907 | 0.20563 | −0.4516 | 0.93364 |
| TBCD \|HSPA1B \|ASCC2 \|PSMA1 \|MTO1 \|DAPP1 \|MAP4K2 \|SLC25A5 \|GMPR2 \|BANP | 5.788089152 | 0.28214 | 0.84032 | 0.18881 | −0.9635 | 0.93052 |
| CNST \|COMMD1 \|TRAK1 \|IKZF3 \|PPM1K \|CLIP4 \|FGD4 \|MRPL3 \|ARL17A \|PDS5A | 5.781315949 | 0.24559 | 0.90538 | 0.13111 | 0.07427 | 0.94254 |
| COMMD6 \|LGALS3 \|UQCRC1 \|ZNF346 \|FBXO9 \|FECH \|PIK3CG \|PRKX \|RGS9 \|RAB3D | 5.774480745 | 0.27263 | 0.9686 | 0.06234 | −1.0572 | 0.93351 |
| ZNF410 \|CAMK2G \|HSD17B12 \|ANKRD49 \|NDUFAF3 \|IMMT \|CNPY2 \|RHOT2 \|SIPA1L3 \|USP22 | 5.766392083 | 0.29636 | 0.85651 | 0.09592 | −0.5608 | 0.92454 |
| KIF1B \|TRAFD1 \|FAM172A \|CCDC93 \|UBTF \|PPP2R1A \|ZC3H7A \|DDX50 \|SLAMF6 \|FAM32A | 5.762340269 | 0.28451 | 0.73709 | 0.20969 | −0.5153 | 0.93591 |
| SDHAF2 \|TMEM219 \|NOD2 \|PATL2 \|FAM122B \|CARS2 \|GCA \|TPM2 \|AEBP1 \|KIAA0141 | 5.758315016 | 0.26492 | 0.83605 | 0.13211 | 0.88251 | 0.92028 |
| SEL1L \|PRDM1 \|AP2A1 \|METAP1 \|GOLPH3 \|MTMR10 \|DHDDS \|ATP6V1C1 \|KCNQ1 \|SPON2 | 5.753302724 | 0.27969 | 0.5797 | 0.2939 | 0.33562 | 0.92494 |
| MAPKAPK5 \|SNRPC \|LZTR1 \|LPCAT1 \|VEZT \|FCGR1A \|RBM22 \|MAU2 \|IP6K2 \|FBXW7 | 5.747104131 | 0.29421 | 0.7604 | 0.20144 | −0.3946 | 0.92625 |
| KIR2DL3 \|ROGDI \|PLA2G6 \|USMG5 \|ATXN3 \|BCAP31 \|PHACTR2 \|ORM2 \|GPN1 \|CD4 | 5.741940186 | 0.27694 | 0.67819 | 0.26674 | −0.8059 | 0.9277 |
| ELF2 \|HIST2H3D \|BROX \|FAM214A \|ZNF480 \|GAPVD1 \|PHF20 \|RNASE6 \|MRPL9 \|SEC11A | 5.734799524 | 0.30469 | 0.76753 | 0.15134 | −0.4836 | 0.93427 |
| GALNT10 \|SCAF8 \|COMMD3-BMI1 \|ARFRP1 \|ATP1B3 \|ZNE808 \|PTBP2 \|CITED2 \|RHBDD1 \|ANXA1j | 5.72924459 | 0.29858 | 0.60585 | 0.31828 | −0.2701 | 0.92156 |
| UBE2A \|CA5B \|RAB24 \|CMTM3 \|PRKACA \|CTNND1 \|ZNF564 \|CUL3 \|ARL5A \|PDLIM2 | 5.724154605 | 0.29007 | 0.82987 | 0.12108 | −0.7249 | 0.91718 |
| PCCB \|GOSR1 \|DHPS \|USP48 \|HIPK2 \|RSL24D1 \|ACO2 \|PSMA7 \|TLR1 \|NFAT5 | 5.716429798 | 0.2961 | 0.66335 | 0.27567 | −0.9651 | 0.94429 |
| SLFN12L \|THUMPD1 \|TNK2 \|SERPING1 \|RNF114 \|FES \|COX7A2L \|FAM13A \|TMEMS5A \|AIMP1 | 5.709802212 | 0.26549 | 1.09025 | 0.06865 | −0.3951 | 0.92987 |
| PRKCQ \|RANBP1 \|CBX4 \|DTNBP1 \|TXN2 \|SLC25A13 \|JTB \|CCDC84 \|DNAJC17 \|CLPTM1 | 5.70297828 | 0.2902 | 0.58616 | 0.2927 | −0.6787 | 0.93038 |
| STAG1 \|DOCK5 \|SEC14L1 \|FOXK2 \|LTB \|EIF3J \|RHOQ \|FAM104A \|EIF2AK1 \|MED24 | 5.697946738 | 0.28838 | 0.5866 | 0.30181 | −1.0189 | 0.92492 |
| KLF3 \|FANCD2 \|BHLHE40 \|STK25 \|DDX1 \|SMC4 \|PAPOLG \|SLC11A2 \|OXCT1 \|GALC | 5.693111709 | 0.27188 | 0.90324 | 0.12009 | −0.3782 | 0.93264 |
| PPP1R7 \|COX20 \|LAT2 \|SH2D1A \|SRCAP \|MBP \|RAB14 \|RHEB \|ATXN1 \|ST3GAL5 | 5.687871965 | 0.29421 | 0.8647 | 0.06062 | −0.2381 | 0.9168 |
| PREPL \|SPDYE3 \|SIK3 \|PITRM1 \|RIMKLB \|GDAP2 \|CHTF8 \|FCHSD1 \|EIF284 \|TRMT1 | 5.682600774 | 0.27041 | 0.90828 | 0.06935 | −0.0988 | 0.92149 |
| XPOT \|GAA \|DENND1C \|YLPM1 \|TFCP2 \|ODF2 \|NOSIP \|ERCC6L2 \|KLRG1 \|ZNF736 | 5.677715906 | 0.27454 | 0.89488 | 0.07872 | 0.16522 | 0.93217 |
| ATF1 \|PCCA \|PEX19 \|SERINC5 \|ELOVL5 \|ARHGAP27 \|MED17 \|ARFGAP1 \|CEP85L \|GLIPR2 | 5.669468763 | 0.26736 | 0.84168 | 0.12074 | −0.7619 | 0.93098 |
| IL24 \|TGB7 \|ERN1 \|MED4 \|PFKFB3 \|MRPL43 \|TBC1D9B \|CEP250 \|C19orf38 \|SMURF2 | 5.661128826 | 0.2605 | 0.83514 | 0.09574 | −0.8592 | 0.91961 |
| NDUFB5 \|MAF1 \|REEP5 \|ULK2 \|STAT5A \|ANAPC7 \|DIAPH2 \|TCEAL4 \|GIMAP6 \|PRR14L | 5.654568935 | 0.27646 | 0.95303 | 0.10613 | −0.5512 | 0.93337 |
| ISY1 \|VHL \|CNDP2 \|CEP95 \|PLCL2 \|RNF216 \|ITPA \|VAPB \|CWC15 \|RRBP1 | 5.648678414 | 0.28976 | 0.7094 | 0.22809 | −0.0439 | 0.93357 |
| STRAP \|METTL3 \|CXorf65 \|BCR \|SLMAP \|SKAP1 \|TMEM175 \|CHSY1 \|PKD2 \|KAT6A | 5.644589648 | 0.27951 | 0.71611 | 0.1812 | 0.16273 | 0.92395 |
| COPS4 \|CD82 \|PIP4K2C \|ST3GAL3 \|SMARCA5 \|ZNF83 \|AARS \|SIRPG \|SPINT2 \|NOL12 | 5.63856602 | 0.26097 | 0.86426 | 0.10747 | −0.5841 | 0.92732 |
| ACP5 \|SLCO3A1 \|SSR3 \|KIAA0513 \|NCK2 \|SLC25A38 \|TMEM104 \|ZMYND8 \|GRPEL1 \|NCBP1 | 5.633675639 | 0.28163 | 0.81606 | 0.128 | −0.5636 | 0.91951 |
| ANKLE2 \|ZNF675 \|PPP2R2A \|FOXN2 \|GSDMD \|ALDH1A1 \|STX3 \|RRM1 \|ARPC5 \|ERC1 | 5.629235973 | 0.27384 | 0.81472 | 0.17501 | −0.5635 | 0.93176 |
| MPI \|HDAC2 \|AAGAB \|PGGT1B \|SYTL2 \|TCHP \|ATP5G1 \|ZDHHC5 \|MAP4 \|BCL2A1 | 5.624566511 | 0.27019 | 0.8973 | 0.13222 | −0.9539 | 0.9383 |
| GALNT7 \|RABGGTB \|CAST11 \|UIMC1 \|TBL1XR1 \|SLC35C2 \|COG3 \|RB1 \|RDX \|WDR41 | 5.620841799 | 0.29027 | 0.89218 | 0.1852 | −0.6247 | 0.93507 |
| DAPK1 \|TAPBPL \|LY6E \|TMEM154 \|MARK2 \|C1orf43 \|NDUFA9 \|TBC1D22A \|THOC3 \|GPR183 | 5.616734582 | 0.26446 | 0.87162 | 0.18071 | −1.1094 | 0.9228 |
| ZBTB16 \|ROMO1 \|CTCF \|ANTXR2 \|YKT6 \|ODC1 \|IL1B \|MCPH1 \|DDX10 \|CCR7 | 5.614284367 | 0.26863 | 0.76275 | 0.11487 | −0.8977 | 0.92978 |
| FAM91A1 \|IMP4 \|WDR6 \|TFIP11 \|CHCHD3 \|SUN1 \|FLVCR2 \|ANAPC4 \|CBX5 \|BSCL2 | 5.609993164 | 0.28637 | 0.75972 | 0.20542 | −0.8247 | 0.93576 |
| SUGP2 \|ALS2 \|PSTPIP2 \|TRAF7 \|CASK \|PON2 \|DENND3 \|GTF2B \|CHCHD2 \|ZNF100 | 5.60609421 | 0.28626 | 0.96742 | 0.14649 | −0.1858 | 0.93269 |
| ZC3H15 \|SOS2 \|CDC25B \|PPP6R1 \|FTSJ1 \|LSG1 \|BIN3 \|GAB1 \|UBE3C \|CAMTA2 | 5.600026587 | 0.29913 | 0.74206 | 0.21604 | −1.0143 | 0.92234 |
| COQ5 \|ELF4 \|DNAJC11 \|RALGAPB \|GRAMD1A \|ACTR1A \|SUMF2 \|RAD51C \|PPP1R15A \|MAPRE1 | 5.595042799 | 0.30051 | 0.70537 | 0.37249 | −0.5136 | 0.93098 |
| HSPA5 \|SUCLG2 \|PNPLA8 \|SENO1 \|KCMF1 \|ANKRA2 \|SEC24C \|TM9SF4 \|ZNF641 \|ESD | 5.591338884 | 0.27746 | 0.79001 | 0.19242 | −0.4222 | 0.94073 |
| CCDC58 \|SAMSN1 \|KLHDC3 \|CTDSP1 \|EGLN2 \|RELL1 \|ZNF43 \|PIK3IP1 \|LY86 \|MOGS | 5.588047388 | 0.26594 | 0.97225 | 0.1902 | −0.2073 | 0.92991 |
| NCOA1 \|STX4 \|TBL1X \|MRPS22 \|NMI \|CACUL1 \|LILRB4 \|PPM1B \|CXXC1 \|PDESA | 5.584028074 | 0.25841 | 1.14392 | 0.07311 | −0.7302 | 0.91887 |
| ERCC3 \|ARL1 \|PARP9 \|RPGR \|SGK3 \|FANCL \|APTX \|DNAJC19 \|ZKSCAN1 \|TP53BP2 | 5.579404116 | 0.28273 | 0.7741 | 0.25875 | −0.9248 | 0.9365 |
| HIST1H4C \|MLEC \|HMOX2 \|FRMD4B \|ZCCHC10 \|CHMP5 \|PELI2 \|AUH \|CYP1B1 \|SLC22A5 | 5.575259734 | 0.28435 | 0.82594 | 0.09858 | 0.22448 | 0.92871 |
| AZI2 \|PSMC4 \|COPS3 \|GTF3C5 \|GTPBP2 \|C18orf8 \|KLF7 \|C5orf51 \|ELMOD3 \|NAA50 | S.569349792 | 0.31207 | 0.67685 | 0.26164 | −0.6055 | 0.93572 |
| PDHX \|GTPBP1 \|HAGH \|CD300A \|CDK10 \|BSG \|CCT6A \|SF3B5 \|GIMAP2 \|HIBCH | 5.563703259 | 0.28966 | 0.8405 | 0.22864 | −0.6889 | 0.92429 |
| NFE2L1 \|CAMTA1 \|ZNF41 \|CCDC130 \|RJOK1 \|NDUFS7 \|PSMD11 \|PHF10 \|ATP6V0B \|TRMT2A | 5.559183352 | 0.29681 | 0.86174 | 0.02727 | −1.0223 | 0.92618 |
| ACBD5 \|PIPSK1C \|EMC7 \|CAB39 \|RAD17 \|CUL4A \|KCTD5 \|DNTTIP2 \|ZNF692 \|RARS | 5.554520516 | 0.30163 | 0.67472 | 0.31379 | −0.8913 | 0.93385 |
| ATP8A1 \|TBC1D23 \|LRRC8D \|PDE3B \|CEP152 \|RCOR1 \|PRR5L \|PECR \|NUP188 \|AKAP10 | 5.548952638 | 0.27356 | 0.65331 | 0.25408 | −0.843 | 0.92875 |
| TOB1 \|ECHDC1 \|SFXN3 \|ADH5 \|PBX2 \|BID \|LY9 \|EBAG9 \|PRRC2A \|GMIP | 5.543144521 | 0.27168 | 0.85936 | 0.18718 | −0.4139 | 0.92297 |
| KAT5 \|ALG9 \|HIST1H4B \|LPIN1 \|RTF1 \|PARP6 \|UBR3 \|PPM1G \|LFNG \|ATP6V1D | 5.538772815 | 0.27236 | 0.79333 | 0.10581 | −0.1945 | 0.92829 |
| TEX261 \|ETHE1 \|TSC1 \|CAPN1 \|AP3M1 \|MED25 \|ACTL6A \|UBR7 \|BCOR \|CMTM6 | 5.534381337 | 0.29148 | 0.92063 | 0.15762 | −0.8312 | 0.91384 |
| SEC61G \|SUGT1 \|PDIA6 \|RAB2B \|DNAJC16 \|LIG1 \|SSRP1 \|MAML3 \|PHBPDLIM1 | 5.530913391 | 0.28814 | 0.72158 | 0.17143 | −0.79 | 0.93503 |
| PEX3 \|KDM6B \|SZT2 \|RPA1 \|PPIH \|RAB3IP \|PRKRA \|CNOT4 \|ZNF845 \|NUP85 | 5.523901493 | 0.28862 | 0.81538 | 0.15082 | −0.4791 | 0.9326 |
| EXOSC9 \|FOXRED1 \|DCTD \|TBCB \|C18orf25 \|AKR7A2 \|ANGEL2 \|NUP107 \|LEMD3 \|TSPAN32 | 5.515552163 | 0.29049 | 0.66896 | 0.26935 | −0.8762 | 0.92396 |
| PRPF18 \|NHP2 \|CPNE3 \|BIVM-ERCC5 \|DTX3L \|C17orf62 \|TRPM7 \|MKL1 \|ARHGEF9 \|ZNF148 | 5.508708294 | 0.28017 | 0.70148 | 0.2604 | −0.0171 | 0.93823 |
| CD22 \|STK17A \|PEX14 \|SRPRB \|PAN2 \|SNX4 \|KIF22 \|MGRN1 \|TMEM43 \|ZNF28 | 5.503839959 | 0.26012 | 0.77205 | 0.18104 | −0.4003 | 0.92729 |
| LSM7 \|GAB2 \|NUP153 \|KIAA0930 \|ORAI1 \|COPE \|CTNNBL1 \|TLE3 \|DENND1A \|ABTB1 | 5.499327358 | 0.29251 | 0.63643 | 0.13164 | −0.9606 | 0.91963 |
| EDC4 \|CEP164 \|CSNK1G3 \|EXOSC10 \|EYA3 \|COPS7A \|CWF19L2 \|PLB1 \|KLHDC4 \|PRKAG1 | 5.49486155 | 0.27254 | 0.93785 | 0.17067 | −0.3454 | 0.93555 |
| TFRC \|KRCC1 \|PTPN7 \|INNT3 \|PEX1 \|POLR2G \|DNAJB1 \|HDAC6 \|RBM19 \|MRRF | 5.491981224 | 0.2846 | 0.89504 | 0.09872 | −0.0231 | 0.92164 |
| MMADHC \|LAIR1 \|POLG2 \|MAN1A2 \|PCED1A \|EXOC7 \|ASCC3 \|CAPRIN2 \|GSN \|SNRPF | 5.487316259 | 0.27526 | 0.73914 | 0.17532 | −0.2589 | 0.9325 |
| RAD52 \|UHRF1BP1L \|RPA2 \|AKR1C3 \|BANK1 \|AHCTF1 \|NAGA \|NUTF2 \|RAB28 \|EGLN1 | 5.481135675 | 0.27698 | 0.85723 | 0.07378 | 0.16216 | 0.94434 |
| INTS6 \|EML2 \|ZNF362 \|GLOD4 \|CWF19L1 \|NUCKS1 \|SLC30A7 \|KCTD20 \|NUP210 \|ENGASE | 5.475605453 | 0.28225 | 0.83385 | 0.17849 | −0.4445 | 0.91775 |
| ZBP1 \|HDDC2 \|NTAN1 \|RWDD1 \|ZC3HAV1 \|SUCO \|DCAF7 \|KDM1B \|RAB5C \|SMPD4 | 5.470374719 | 0.29555 | 0.67656 | 0.13015 | 0.16903 | 0.92537 |
| AFTPH \|POC1B \|EBP \|PLOD1 \|VTA1 \|SE3A2 \|SLC43A2 \|WDR75 \|INTS4 \|PPP4R1 | 5.463744075 | 0.30833 | 0.74636 | 0.11318 | 0.40138 | 0.92972 |
| FAM177A1 \|CLIP1 \|RUFY3 \|VPS16 \|QRICH1 \|SLC35B1 \|UPP1 \|ACADSB \|PJA2 \|LY96 | 5.455574412 | 0.26723 | 0.99727 | 0.10427 | −0.6615 | 0.93642 |
| DHTKD1 \|TNFRSF14 \|PLCD1 \|MUS81 \|S100PBP \|DDX52 \|SCARB2 \|FCHO1 \|DDX41 \|COMMD10 | 5.450993618 | 0.29321 | 0.92999 | 0.15966 | −1.018 | 0.91829 |
| RNF123 \|MEF2D \|COPS6 \|TOP1 \|PRKAR2B \|MCOLN1 \|IBTK \|PWP1 \|C6orf136 \|PPP2R3C | 5.44604185 | 0.28593 | 0.57452 | 0.31021 | −1.0074 | 0.92491 |
| IRAK1 \|RSRC1 \|LILRA4 \|EPN1 \|OSBPL3 \|TM7SF3 \|C16orf62 \|RRAS2 \|CACYBP \|RGCC | 5.438671574 | 0.26801 | 0.74236 | 0.179 | 0.40111 | 0.92603 |
| CSF2RB \|ADHFE1 \|AMPD3 \|UPF2 \|C4A \|ALG1 \|HIST1H2AG \|SLC4A7 \|EI24 \|FAM193A | 5.432895571 | 0.24121 | 1.0945 | 0.07248 | −0.7638 | 0.9425 |
| ARPC1A \|TDP2 \|DCAF17 \|SLC25A12 \|WRNIP1 \|GSK3A \|COX5A \|DPEP2 \|VPS11 \|CCDC14 | 5.430150829 | 0.28464 | 0.677 | 0.2219 | −0.8023 | 0.93467 |
| ATP8B2 \|ZNF76 \|EXOSC3 \|PLXND1 \|AHSA2 \|KLHDC2 \|UXS1 \|MRPS16 \|PEX5 \|GTF2E1 | 5.423477986 | 0.28216 | 0.7049 | 0.24142 | −0.6721 | 0.92376 |
| DDA1 \|NABP1 \|GK5 \|UCHL5 \|MCM3 \|FEM1B \|RALY \|ARL8B \|LMNA \|PANK3 | 5.415941045 | 0.29465 | 0.68049 | 0.19064 | −0.9719 | 0.9297 |
| FCAR \|ACLY \|TTC1 \|NFX1 \|MAP2K4 \|ETS2 \|MTF2 \|ELMOD2 \|SFMBT2 \|C1orf131 | 5.410902162 | 0.26818 | 0.82054 | 0.1223 | −0.6378 | 0.93855 |
| XIAP \|FCRL6 \|SH2B1 \|FBXO22 \|NFU1 \|IPO9 \|SWAP70 \|KLRB1 \|MFF \|CREBRF | 5.407496245 | 0.25498 | 0.88715 | 0.09488 | −0.4905 | 0.93017 |
| TRPC4AP \|H2AFV \|CCDC50 \|CCT4 \|TBC1D5 \|TLR8 \|CDA \|SLC15A3 \|TRAPPC12 \|HSD17B10 | 5.40380087 | 0.26234 | 1.11315 | 0.06402 | −0.9198 | 0.93594 |
| ALG8 \|POLR1C \|ASL \|BBS9 \|MX1 \|GPHN \|CDK7 \|GOLGA6L4 \|ZNF655 \|MTX1 | 5.398773232 | 0.28552 | 0.7718 | 0.26599 | 0.1203 | 0.9342 |
| TADA2A \|BTBD1 \|EXOSC8 \|TMEM185A \|LRTOMT \|HIST1H2BG \|LRSAM1 \|EML3 \|STAB1 \|TRIM44 | 5.394369308 | 0.28177 | 0.84309 | 0.09559 | −0.8388 | 0.91632 |
| SKIV2L \|OTUD5 \|TRNT1 \|TBC1D10C \|ENSA \|MXI1 \|SLC14A1 \|PAM \|RNF20 \|GPR107 | 5.388962557 | 0.26937 | 0.94414 | 0.12004 | 0.62365 | 0.93532 |
| ACBD6 \|TAPT1 \|ITPR1 \|KIAA0556 \|ZNF224 \|CD300E \|GNL3L \|TAF1D \|NFATC2 \|MOV10 | 5.383918733 | 0.26922 | 0.80737 | 0.16353 | −0.0115 | 0.93115 |
| MAPKAP1 \|GMPS \|IQSEC1 \|CBX3 \|PPP1R9B \|CDC16 \|CYB5R4 \|DNAJC2 \|ERICH1 \|UPF3B | 5.378347605 | 0.29823 | 0.68554 | 0.16495 | −0.718 | 0.92984 |
| YAF2 \|ANKRD28 \|DDX55 \|BUD31 \|AREG \|C11orf21 \|MOB3A \|CCNG1 \|FCGR1B \|CD59 | 5.373927948 | 0.28655 | 0.89582 | 0.05872 | −0.7843 | 0.92066 |
| ZNF763 \|EEA1 \|NUPL2 \|RAD51B \|PDCD5 \|MATK \|AP1B1 \|MGAT2 \|ALKBH3 \|ZCCHC8 | 5.364521469 | 0.27767 | 0.78393 | 0.19932 | 0.58263 | 0.93365 |
| VOPP1 \|DIP2A \|CDK8 \|BST2 \|GTF2H2 \|ZCRB1 \|MEN2 \|SLC24A1 \|TTC31 \|THOC1 | 5.356573776 | 0.26734 | 0.86809 | 0.16513 | −0.8612 | 0.92324 |
| USP5 \|KPNA1 \|IGSF6 \|RP9 \|FAM153B \|MBNL2 \|KLHL2 \|AIDA \|CCNT1 \|TMX3 | 5.351974299 | 0.27255 | 1.00839 | 0.03727 | −0.5482 | 0.94742 |
| LAMTOR1 \|PPDPF \|AGER \|SNRPA \|ABCD4 \|COG8 \|RPAP3 \|PTAR1 \|ZNF562 \|MCCC1 | 5.347702412 | 0.31075 | 0.65163 | 0.33208 | −1.0248 | 0.93403 |
| RUFY2 \|FGD2 \|CTSK \|PDIA4 \|PPIE \|JAK3 \|COPS2 \|AP4M1 \|DPP9 \|DNAJA2 | 5.343075072 | 0.27934 | 0.85014 | 0.12934 | 0.13317 | 0.9287 |
| DCTN5 \|CHCHD10 \|USP47 \|POMT2 \|GP1BA \|TMX4 \|BCL3 \|HMGCR \|UTP14A \|EXOSC1 | 5.33682202 | 0.27937 | 0.84166 | 0.18849 | −0.8008 | 0.92706 |
| EXT2 \|ANKRD27 \|GLE1 \|IFI35 \|WDR44 \|GLRX3 \|GGA1 \|NMD3 \|UVRAG \|PIP4K2B | 5.329607023 | 0.29614 | 0.88947 | 0.15774 | −1.0117 | 0.92026 |
| STK11 \|RNPEP \|BRD8 \|LETMD1 \|PRKAR2A \|TMEM134 \|GTF2E2 \|SNX27 \|LATS1 \|PEX2 | 5.326555067 | 0.28651 | 0.71501 | 0.21937 | −0.9767 | 0.93226 |
| SLU7 \|CPNE8 \|METTL7A \|WDR3 \|TMEM14B \|SULT1A1 \|SYTL3 \|SEC24D \|THYN1 \|ARL14EP | 5.323250991 | 0.25703 | 0.96396 | 0.08661 | 0.21672 | 0.93717 |
| TMEM87B \|SPAG7 \|BBP1 \|FARSA \|DEGS1 \|SMAD5 \|ZNF81 \|SRP19 \|ATP6AP1 \|IDI1 | 5.317711277 | 0.29567 | 0.66974 | 0.29491 | −0.1961 | 0.93296 |
| SERGEF \|NVL \|WIPI2 \|ARFGEF1 \|MAPK8 \|GSTM4 \|KLRC3 \|ACER3 \|P2RX5 \|TMEM126B | 5.311619423 | 0.28115 | 0.78182 | 0.24778 | −0.7685 | 0.93009 |
| ZDHBC18 \|ARF4 \|ZNF880 \|FCER1A \|CCDC117 \|NSUN2 \|HMBS \|IWS1 \|IDH3A \|ING4 | 5.304888224 | 0.28494 | 0.69001 | 0.24245 | −0.864 | 0.93552 |
| GSDMB \|RIN3 \|LCOR \|DHODH \|SLC35A1 \|TM9SF3 \|PSMG2 \|PDLIM5 \|NFKBID \|EIF282 | 5.301902278 | 0.27035 | 0.87555 | 0.13318 | −0.501 | 0.92826 |
| LY75-CD302 \|IFT122 \|UBR2 \|NECAP1 \|LTV1 \|ZNF292 \|PDZK1 \|NME8 \|XBP1 \|SLC30A6 | 5.298333969 | 0.28046 | 0.83557 | 0.14405 | −0.4709 | 0.93946 |
| TMEM161B \|DCUN1D1 \|PEX16 \|RGS3 \|DHX40 \|GBP4 \|ZNF266 \|RHBDF2 \|EIF2D \|LSS | 5.293527738 | 0.26047 | 0.92276 | 0.16283 | −0.4855 | 0.93065 |
| PICK1 \|STYXL1 \|BRK1 \|RAP1GDS1 \|ARSB \|HSPE1 \|TBC1D9 \|PAIP1 \|BCS1L \|PZP | 5.287403487 | 0.28266 | 1.0173 | 0.06089 | −0.8181 | 0.93263 |
| UBE4B \|MCTP2 \|ZNF24 \|PDE4B \|NNT \|PHF19 \|DAXX \|AGK \|SH2D3C \|SEC11C | 5.282141706 | 0.25987 | 1.17126 | 0.04406 | −0.6023 | 0.93178 |
| TRMT1L \|WBP1 \|ADAMTSL4 \|AFG3L2 \|IL12RB1 \|UNC119 \|ITGA2B \|GABPB2 \|SUPT16H \|DCAF13 | 5.278408227 | 0.28814 | 0.89863 | 0.11916 | −0.4376 | 0.92166 |
| C12orf75 \|ADAM17 \|NEDD9 \|RBBP6 \|CIAO1 \|EPSTI1 \|GLCCI1 \|RNF214 \|TIMM17A \|SLC40A1 | 5.272299909 | 0.29032 | 0.77386 | 0.18262 | −0.6337 | 0.93227 |
| TACC3 \|PDGCR2 \|ERAL1 \|DIS3 \|MSRB1 \|PPCS \|CLEC4A \|TSC22D2 \|MS4A14 \|P2RX7 | 5.266975528 | 0.26915 | 0.88722 | 0.09606 | −0.3225 | 0.93415 |
| TAF8 \|PER1 \|C3orf38 \|PPP4C \|IMMP2L \|DCUN1D4 \|STAM2 \|PTOV1 \|UVSSA \|TOR1A | 5.263775793 | 0.3028 | 0.73921 | 0.17006 | −0.7714 | 0.92947 |
| OTUD4 \|FANCA \|AMD1 \|DBP \|SENP7 \|RXRA \|ZC3H6 \|PHF14 \|SGK494 \|ZP3 | 5.258969081 | 0.26747 | 0.80011 | 0.11551 | −0.8249 | 0.91925 |
| SIGLEC7 \|DCTN6 \|BCAS2 \|JAK2 \|ATAD3B \|YTHDC2 \|PHTF2−DNAJA3 \|SPEN \|NUP160 | 5.251081964 | 0.2715 | 0.85245 | 0.16559 | −0.7265 | 0.93921 |
| SCMH1 \|MLLT3 \|ZNF700 \|ARSG \|PRMT7 \|VIPR1 \|MALT1 \|FGFR1OP2 \|MIB1 \|CD38 | 5.245126485 | 0.25605 | 0.90535 | 0.11519 | −0.8559 | 0.93205 |
| MITD1 \|LARP4B \|RRN3 \|HAVCR2 \|CD1C \|TOPORS \|SUSD1 \|SLC39A11 \|TRIM24 \|SUMF1 | 5.23854208 | 0.25194 | 1.1126 | 0.12239 | −0.6383 | 0.94291 |
| ATP13A2 \|ENTPD6 \|INO80B \|RNF166 \|RPRD2 \|RABGGTA \|PLD2 \|TXNL1 \|SLC25A37 \|MLKL | 5.229804142 | 0.29696 | 0.70728 | 0.25378 | −1.0143 | 0.91838 |
| CARD11 \|RAE1 \|BCL2L11 \|PARVB \|CCDC28A \|RNASEH2C \|NCAPD2 \|CSGALNACT2 \|NSMCE2 \|HAL | 5.224706778 | 0.25224 | 0.7194 | 0.24651 | −0.7164 | 0.93228 |
| STX5 \|SMARCD2 \|BCCIP \|REV1 \|GSS \|VPS72 \|DDX60 \|DDX56 \|POLR3C \|ATAD3A | 5.220540832 | 0.29466 | 0.75547 | 0.1667 | −0.1767 | 0.93214 |
| UQCRQ \|OXSR1 \|ZC3H8 \|RBX1 \|PBXIP1 \|MGAT1 \|SAT2 \|DHRS4L2 \|CYBRD1 \|ZBTB44 | 5.216899996 | 0.29874 | 0.76535 | 0.291 | −0.3205 | 0.93097 |
| EPG5 \|ADAP2 \|BIST1H4L \|SAMD9L \|GALK2 \|ATR \|CUL1 \|KIF5B \|RERE \|RBM4B | 5.212643998 | 0.28269 | 0.96703 | 0.04307 | −0.1141 | 0.94205 |
| PHF23 \|TBC1D3C \|FOSB \|TXNDC12 \|CDK2AP1 \|ADSS \|FAM126B \|CXorf56 \|ZNF333 \|GTF2F1 | 5.207353401 | 0.29509 | 0.8241 | 0.17463 | −0.9829 | 0.93148 |
| ATG2B \|BAZ1B \|N4BP1 \|TTYH2 \|PPP2R5D \|FAM13B \|STX17 \|SRSF9 \|GPD1L \|ZNF264 | 5.20278423 | 0.29057 | 0.71227 | 0.28832 | 0.32511 | 0.92082 |
| CRNKL1 \|ABCA7 \|RRM2B \|TRIT1 \|SNX30 \|LARS2 \|LDLR \|LMO7 \|BBS1 \|SH3GL1 | 5.198613099 | 0.26443 | 0.82763 | 0.13319 | −0.2842 | 0.9335 |
| CCDC25 \|FYCO1 \|GPBAR1 \|CSNK2A2 \|ARHGAP12 \|ADNP \|ECI2 \|DAP \|PLAA \|MCMBP | 5.191143738 | 0.28734 | 0.68653 | 0.18091 | −0.936 | 0.92454 |
| IGBP1 \|ILKAP \|SCAMP1 \|TFDP2 \|ZNF493 \|PPP1R8 \|PRPF19 \|GORASP1 \|DDX49 \|DTD1 | 5.186885166 | 0.28337 | 0.73893 | 0.26112 | −0.3557 | 0.93189 |
| ADRM1 \|KIAA2026 \|MAK16 \|SLC37A3 \|SAP30L \|ACACA \|C14orf119 \|PEX26 \|AKIRIN1 \|RAB1B | 5.180786729 | 0.29952 | 0.73127 | 0.14772 | 0.02005 | 0.92332 |
| ISG20L2 \|NUP155 \|NAA15 \|MTA1 \|SLC39A9 \|VAMP1 \|TFB1M \|IGLL5 \|VAMP5 \|FAM114A2 | 5.176221843 | 0.27633 | 0.82862 | 0.18404 | −0.6249 | 0.92793 |
| RELA \|MEN1 \|DIP2B \|QPCT \|ZNF217 \|PTS \|CNOT6 \|SYK \|MMAB \|ZNF124 | 5.172870147 | 0.28181 | 0.8052 | 0.11003 | −0.2273 | 0.92855 |
| PGLS \|MYOM2 \|PSME4 \|CIC \|GPSM3 \|FAM149B1 \|RFWD3 \|COX11 \|SIRT2 \|ETNK1 | 5.167884761 | 0.28068 | 0.67953 | 0.18837 | −0.5028 | 0.93127 |
| SDF4 \|LLPH \|SLC39A10 \|RFT1 \|DHX16 \|PRKD3 \|PES1 \|HERC4 \|GLT8D1 \|ENOSF1 | 5.162552793 | 0.2753 | 0.77794 | 0.1338 | −0.4601 | 0.94585 |
| PHF6 \|ZNF141 \|DLGAP4 \|TM2D1 \|OAT \|MFN1 \|MED1 \|NLK \|DENND4C \|ZBTB38 | 5.154709674 | 0.26845 | 0.85825 | 0.08804 | −0.1354 | 0.92615 |
| ZNF211 \|STRADA \|NGDN \|ZC3H18 \|MAD1L1 \|PPIL4 \|CLTB \|LYAR \|TRIM34 \|HYOU1 | 5.15021895 | 0.2771 | 0.56806 | 0.24204 | 0.30283 | 0.93012 |
| GOLGA1 \|FCHO2 \|TSC22D4 \|TRIM14 \|CEP192 \|DPP8 \|ATP5G3 \|KCTD10 \|DEAF1 \|FBXO3 | 5.145966823 | 0.27945 | 0.78242 | 0.18665 | −0.8288 | 0.92913 |
| TXNDC11 \|ATXN7L3 \|PLA2G7 \|TMEM165 \|PCGF1 \|SPCS3 \|TCP11L2 \|SRP68 \|RNGTT \|CD2AP | 5.139718918 | 0.27992 | 1.00995 | 0.05 | −0.0724 | 0.93433 |
| ATF6 \|YPEL3 \|OGFOD1 \|TMEM19 \|TFG \|BIRC3 \|THBS1 \|KIF13B \|IRAK4 \|SUPV3L1 | 5.135161907 | 0.29303 | 0.76829 | 0.15738 | −0.4928 | 0.93968 |
| PHAX \|MEAF6 \|FLAD1 \|WDR46 \|RPL7L1 \|RPAIN \|CKAP5 \|POLR2C \|EGR2 \|PPP4R2 | 5.129021919 | 0.27752 | 0.91414 | 0.11675 | −0.9366 | 0.93726 |
| TFDP1 \|RAB11FIP2 \|TMEM245 \|MRPL52 \|PHACTR4 \|TNKS \|PDSS1 \|STAU1 \|TTC27 \|CUTA | 5.123915837 | 0.27971 | 0.68962 | 0.26209 | −0.7944 | 0.93117 |
| TPGS2 \|SENP5 \|KLBL5 \|FKBP5 \|F11R \|GTF3C1 \|STAMP \|HP \|FAM156A \|HKR1 | 5.118750883 | 0.26483 | 0.90127 | 0.15475 | −0.7876 | 0.92931 |
| CLUAP1 \|RABL2B \|CSNK1G1 \|TM2D2 \|NMRAL1 \|AP5M1 \|G2E3 \|U2AF2 \|PRKRIP1 \|ST3GAL6 | 5.114525013 | 0.29776 | 0.73937 | 0.25719 | −0.6871 | 0.92452 |
| SLC30A9 \|PPP1R21 \|ZBTB48 \|DNTTIP1 \|NANS \|RAI1 \|IL1RAP \|TCTN1 \|CDK5RAP1 \|PRPSAP2 | 5.111093933 | 0.28765 | 0.59236 | 0.28825 | −0.2214 | 0.93322 |
| ZNF101 \|RCC2 \|MORF4L2 \|SIDT1 \|SLC27A3 \|HIST1H1B \|PRDX6 \|STAMBP \|CLEC17A \|PDPK1 | 5.106097 | 0.27398 | 0.90577 | 0.14505 | −0.8676 | 0.92747 |
| CCDC144A \|TIMM10 \|ACAD8 \|PRKDC \|TBC1D2B \|TRMU \|PPID \|ARHGEF11 \|KANSL2 \|TRAT1 | 5.101614442 | 0.27078 | 0.85506 | 0.0719 | −0.4908 | 0.94178 |
| TRIM37 \|HUS1 \|SENP1 \|SMARCD1 \|RHBDD2 \|TASP1 \|SLC35A5 \|AZIN1 \|IKBKE \|ADAM15 | 5.0978025 | 0.28751 | 0.8983 | 0.17766 | −0.0243 | 0.93296 |
| ERCC2 \|SERINC1 \|DTNB \|ZFYVE19 \|CFL2 \|ATP6V1F \|DIMT1 \|WDR61 \|TMEM209 \|DPY19L1 | 5.093346222 | 0.27866 | 0.85875 | 0.16855 | −0.5574 | 0.94045 |
| DCAF6 \|MRPS24 \|NOL7 \|KDM2B \|BMPR2 \|VSIG1 \|PDE4D \|MTMR4 \|CYC1 \|HK2 | 5.088896603 | 0.26921 | 0.87274 | 0.15049 | −0.7674 | 0.92674 |
| GTDC1 \|FPGS \|EXOC6 \|NEXN \|SLK \|ZNF417 \|TRMT2B \|EPS15L1 \|MRPL48 \|MTPAP | 5.082552739 | 0.26818 | 0.78643 | 0.15878 | −0.9892 | 0.93838 |
| ABHD10 \|POLR2K \|STXBP5 \|TTC12 \|NOL10 \|TIMM44 \|PFKM \|NDFIP1 \|SGTB \|SMARCD3 | 5.077741481 | 0.28727 | 0.98412 | 0.14744 | −0.9144 | 0.93818 |
| EIF5B \|ORC2 \|DUSP22 \|SLC9A9 \|TNPO3 \|THADA \|SIN3A \|FKBP8 \|MBNL3 \|CNTROB | 5.074772935 | 0.28613 | 0.74668 | 0.22319 | 2.29511 | 0.94352 |
| FBXO33 \|BRPF1 \|BSDC1 \|ACSL3 \|SLC33A1 \|GOLGA2 \|ZNF611 \|EP400 \|INF2 \|FZR1 | 5.06880303 | 0.28461 | 0.50218 | 0.25109 | 0.45728 | 0.91469 |
| HNRNPH2 \|HIGD2A \|DPY30 \|FKBP3 \|COMMD9 \|SARS2 \|TSTD1 \|TTC38 \|MPHOSPH10 \|WDR20 | 5.064164281 | 0.28389 | 0.64425 | 0.29172 | −0.9168 | 0.93772 |
| CCNC \|SLC6A6 \|SIRPA \|STX18 \|ACSL4 \|ZNF131 \|PHIP \|LIN54 \|CLPP \|SLC35F5 | 5.059704948 | 0.28912 | 0.6709 | 0.31144 | −0.9605 | 0.92822 |
| HIRA \|OR2A7 \|SLFN11 \|EPC2 \|POUR2F \|RALGAPA2 \|BAG1 \|CD58 \|IGHMBP2 \|PDP1 | 5.054739555 | 0.27016 | 0.73314 | 0.16499 | 0.15668 | 0.92729 |
| CEP120 \|SERPINB8 \|AGGF1 \|ING3 \|GIT1 \|CPEB4 \|TNFRSF25 \|DISC1 \|SYTL1 \|TTC39C | 5.050427166 | 0.26229 | 0.82506 | 0.12849 | −0.8095 | 0.92623 |
| STK39 \|DEF8 \|LMNB1 \|TRIP4 \|KDM1A \|SKI \|SNRPB2 \|ALG6 \|SYPL1 \|DAAM1 | 5.045827417 | 0.28258 | 0.7979 | 0.20336 | −0.129 | 0.93844 |
| PAPD7 \|CWC27 \|DPAGT1 \|ARHGAP31 \|DHX8 \|RAB12 \|RPF1 \|ZBTB1 \|TCF20 \|PACSIN2 | 5.041579949 | 0.27522 | 0.92084 | 0.03238 | −0.1053 | 0.93165 |
| DDX54 \|C1GALT1 \|APOL2 \|NAA25 \|ATP5J \|HERC2 \|TROVE2 \|RALA \|ZFAND2B \|FARS2 | 5.037080512 | 0.2874 | 0.82708 | 0.1792 | −0.8039 | 0.93207 |
| KLHL18 \|LYPLA2 \|DUSP11 \|POLR2H \|TMEM199 \|SLAMF7 \|UBE2Z \|TOM1L2 \|UBA5 \|TXNRD2 | 5.032785068 | 0.27406 | 0.89289 | 0.18623 | −0.1934 | 0.92054 |
| FCRL3 \|HARS \|EPHX2 \|RPS6KB2 \|RDH11 \|ZNF92 \|FAM3C \|CHD1L \|NDUFA5 \|CPT1A | 5.027823353 | 0.26427 | 0.8902 | 0.14498 | −0.3472 | 0.93946 |
| ACSS2 \|PCID2 \|IPO7 \|POLB \|ATF7IP2 \|LRPAP1 \|HNRNPAB \|MTIF2 \|IFI44L \|FUBP3 | 5.022195773 | 0.27489 | 0.79335 | 0.21824 | −0.8386 | 0.93507 |
| DNPEP \|CDK16 \|PPM1F \|TRIM21 \|VEGFA1 \|IFI27L2 \|TSEN2 \|PLCB1 \|WDR74 \|PPP2R5A | 5.014338252 | 0.26916 | 0.97624 | 0.10568 | −0.9689 | 0.92621 |
| TTC9C \|ZNF302 \|REEP3 \|FAM98A \|SSNA1 \|COL4A3BP \|NR4A1 \|SLC25A39 \|TIMM21 \|SLC25A36 | 5.009961376 | 0.27487 | 0.84793 | 0.15078 | −0.8762 | 0.93075 |
| HIP1R \|PSMD9 \|ZNE585B \|DYNC1L11 \|CLEC16A \|NUBP1 \|PCMT1 \|ALDH2 \|CHURC1 \|RIF1 | 5.005391365 | 0.29729 | 0.68065 | 0.17522 | −0.5043 | 0.93648 |
| METTL2B \|AARSD1 \|C16orf58 \|SLC25A46 \|C1orf52 \|GPR108 \|PABPC1L \|ZFYVE16 \|TMEM173 \|NDUFB3 | 5.000840687 | 0.28084 | 0.8482 | 0.27235 | −0.8337 | 0.93146 |
| CIAPIN1 \|ZNF236 \|TARS2 \|IAH1 \|SEH1L \|ITGB3 \|ALMS1 \|FASTKD2 \|UBE2E2 \|CD8B | 4.996679771 | 0.25945 | 0.76687 | 0.16795 | −0.7588 | 0.9227 |
| HSF1 \|FANCI \|YIPF3 \|FAM198B \|TAF1C \|ZNE460 \|RARA \|BTBD7 \|GRSF1 \|PLK3 | 4.992806154 | 0.28329 | 0.79861 | 0.2066 | −0.8027 | 0.9304 |
| KDM4A \|FOLR3 \|SRBD1 \|MIER3 \|SGSM2 \|ARL2BP \|CCL3 \|USP11 \|TP53BP1 \|RHD | 4.98876913 | 0.26067 | 0.80044 | 0.11539 | −0.8479 | 0.93045 |
| PIN4 \|CSGALNACT1 \|CYB5A \|SUGP1 \|ATG5 \|MTFMT \|MAST4 \|EXOC3 \|MR1 \|CPSF3 | 4.984435424 | 0.27152 | 0.78178 | 0.20818 | 4.4064 | 0.9301 |
| SCRN3 \|MLF2 \|MED6 \|RNH1 \|SKA2 \|TMEM205 \|YIPF5 \|KLF10 \|SSU72 \|PIGK | 4.979946268 | 0.2943 | 0.77584 | 0.17076 | −0.7381 | 0.93129 |
| RPP30 \|ERBB2 \|FBP1 \|SCAP \|FAM96B \|TMEM8A \|POLDIP2 \|TMEM106C \|ZFYVE27 \|SH3BP5 | 4.975392131 | 0.26237 | 0.90852 | 0.14852 | 0.33255 | 0.92788 |
| TRNAU1AP \|NEK4 \|RNF145 \|SPIN2B \|UPRT \|MRPL23 \|UTP18 \|SLC43A3 \|HMG20B \|SRSF2 | 4.971902536 | 0.29557 | 0.68046 | 0.17075 | −0.7699 | 0.9167 |
| TIMM50 \|ZNF44 \|MOSPD2 \|TRAPPC5 \|DHX57 \|ATL3 \|GOSR2 \|PIK3R4 \|PRKD2 \|TWF1 | 4.967643582 | 0.29447 | 0.8384 | 0.10824 | −0.1738 | 0.93206 |
| SLC24A4 \|TOMM22 \|ST14 \|LHPP \|POP4 \|GYG1 \|PIGN \|MTPN \|PTAFR \|PYGB | 4.961597139 | 0.25578 | 0.9083 | 0.0798 | −0.8771 | 0.92287 |
| FAM153A \|FBRSL1 \|ZNF254 \|MRPS7 \|SEXN5 \|SEC6LA2 \|BBS7 \|UCKL1 \|RANBP9 \|DCAF12 | 4.956302264 | 0.29078 | 0.79427 | 0.23636 | −0.1385 | 0.92141 |
| SENP3 \|DDRGK1 \|TNRC6C \|EAF2 \|COX6C \|CSNK1E \|SMGS \|TYW1 \|PSMB6 \|UBE2G1 | 4.951556005 | 0.26704 | 0.72188 | 0.15947 | −0.1804 | 0.93524 |
| ATOX1 \|CLSTN1 \|ETF1 \|SNAPC5 \|CHST15 \|SNRNP40 \|HMGCL \|MRPL18 \|LIMD1 \|PAPSS1 | 4.94319673 | 0.28224 | 0.73858 | 0.22612 | −0.6477 | 0.91653 |
| TMED4 \|C2CD2L \|ORMDL3 \|SPATA13 \|TGS1 \|NAA20 \|NSUN6 \|RBCK1 \|FAM60A1 \|IQCB1 | 4.938236452 | 0.27466 | 0.8958 | 0.11065 | −0.7079 | 0.93124 |
| PISD \|MCM7 \|FAM26F \|STK11IP \|FER \|APH1A \|SLC25A30 \|IP6K1 \|ARFGAP3 \|HIST2H2AB | 4.932548868 | 0.28645 | 1.06124 | 0.08924 | 0.07223 | 0.93099 |
| SCYL1 \|SECTM1 \|GOLGA5 \|FAM35A \|DYSF \|MAST3 \|PSMA2 \|ZBTB40 \|ZNF226 \|HEG1 | 4.927317691 | 0.27526 | 0.64089 | 0.17514 | −0.6304 | 0.92286 |
| RIT1 \|IRF4 \|TOPBP1 \|PIGG \|RBM7 \|GBF1 \|TESC \|CC2D1A \|SERP1 \|MED13 | 4.920548103 | 0.28302 | 0.83604 | 0.19648 | −0.0114 | 0.9288 |
| NARF \|CDH23 \|AP3D1 \|REC8 \|PHC2 \|AGTRAP \|MED21 \|USP16 \|BEST1 \|COMT | 4.913691719 | 0.26248 | 0.85908 | 0.17036 | −0.7953 | 0.92314 |
| NEDD1 \|MCU \|PPIL3 \|POLA2 \|ASNA1 \|MCFD2 \|ADRB2 \|TMEM179B \|ECSIT \|ABHD16A | 4.907465648 | 0.30151 | 0.63093 | 0.28877 | −0.748 | 0.9335 |
| EXOSC2 \|C11orf54 \|PPP2R5E \|GOLGB1 \|POR \|MRPL22 \|RHOG \|PRPF39 \|PRR14 \|MRPS21 | 4.903738981 | 0.29194 | 0.63453 | 0.25698 | −0.8007 | 0.93866 |
| SPSB3 \|ZCCHC17 \|PYCARD \|MGST1 \|SAE1 \|PADI4 \|IER3 \|TBC1D25 \|PRDM4 \|ABCF2 | 4.899441445 | 0.26922 | 0.83091 | 0.10609 | −0.211 | 0.92498 |
| TRDMT1 \|PLXNA3 \|TXNDC17 \|PIGU \|MCM3AP \|ZFPL1 \|DCP1B \|BET1L \|ENO2 \|ZNF185 | 4.894355167 | 0.27727 | 0.92585 | 0.10654 | −0.5399 | 0.92073 |
| ARHGAP19-SL1T1 \|XPO5 \|CCDC167 \|BRF1 \|KPNA5 \|BLMH \|KLRC4 \|C4B \|MDM1 \|RRP12 | 4.88996914 | 0.27012 | 0.69894 | 0.21518 | −0.531 | 0.94016 |
| TRMT112 \|KLHL24 \|TBC1D13 \|HSPB11 \|TRIB2 \|DUT \|SMC6 \|RECQL \|ENG \|BOLA2 | 4.88573341 | 0.29138 | 0.65688 | 0.33901 | −0.4879 | 0.91975 |
| PTPRO \|TMF1 \|CCL4L2 \|ALG5 \|OTUD6B \|HSPH1 \|ZNF160 \|IL13RA1 \|CWC22 \|ACAD10 | 4.881481452 | 0.27473 | 0.73632 | 0.16491 | −0.601 | 0.93991 |
| POLR2I \|TIPRL \|CD69 \|STOML2 \|PLXDC1 \|POU2F1 \|APOBEC3C \|POLE \|MARK4 \|LSM14B | 4.876216478 | 0.28384 | 0.721 | 0.17368 | −0.1033 | 0.91153 |
| MSB5 \|UBQLN2 \|KIR3DL2 \|TBCID3G \|ACAD11 \|TFEC \|HSBP1 \|ASB8 \|ABHD5 \|SAMD4B | 4.868933324 | 0.27827 | 1.05099 | −0.0019 | −1.1 | 0.93878 |
| BRD1 \|PIAS4 \|NUDCD3 \|SMAP1 \|KANSL3 \|GTPBP10 \|AKAP8 \|HACE1 \|AMT \|ALG10 | 4.863534403 | 0.26618 | 0.85376 | 0.17042 | −0.5357 | 0.93357 |
| CDK14 \|MBD6 \|CDR2 \|GFER \|PML \|RB1CC1 \|CKAP2 \|PSMD3 \|DDX39A \|LMBRD2 | 4.858511671 | 0.29259 | 0.52549 | 0.24279 | −0.2523 | 0.93463 |
| PFKP \|TSPYL1 \|NCOA6 \|ZNF766 \|SMYD5 \|CAMK1D \|GALNT3 \|COMMD2 \|MDC1 \|TMED9 | 4.850648869 | 0.26285 | 0.86526 | 0.20862 | −0.4524 | 0.9332 |
| ZNF841 \|POLR3A \|ZNF720 \|TRAPPC8 \|CDK19 \|CCNG2 \|ZNF84 \|COLQ \|PGM2 \|PLD4 | 4.845533143 | 0.27011 | 0.75297 | 0.18862 | −0.2554 | 0.92142 |
| FXN \|LPCAT3 \|BCL6 \|ZNF277 \|PNPO \|PSMD12 \|OSBP \|ORMDL1 \|THOC5 \|IFT52 | 4.838786146 | 0.30138 | 0.75004 | 0.19703 | −0.6628 | 0.94016 |
| MRPL42 \|AK5 \|CDKN1A \|MRPS18B \|PPP5C \|BPNT1 \|RPP21 \|RNF11 \|TDRD7 \|ABAT | 4.833915474 | 0.25272 | 0.81566 | 0.23839 | −0.8275 | 0.92963 |
| ZNF589 \|KLHL7 \|TRAP1 \|CAND1 \|RAB35 \|DLG3 \|ARHGAP21 \|KIAA0391 \|EIF4A3 \|PAG1 | 4.82898719 | 0.29016 | 0.63915 | 0.35173 | −0.8148 | 0.92477 |
| CERS5 \|FTSJ3 \|SGSM3 \|JUNB \|JMJD8 \|ZNF513 \|SH3YL1 \|DNAJB11 \|MTHFR \|CLEC4E | 4.825334389 | 0.28609 | 0.83288 | 0.13827 | −0.6424 | 0.93191 |
| BBIP1 \|COX10 \|NHEJ1 \|CHUK \|DDX27 \|CNOT3 \|SVIL \|FKTN \|AMN1 \|PPP6R2 | 4.820591529 | 0.28567 | 0.90597 | 0.18048 | −0.2435 | 0.93121 |
| FAM96A \|SNX10 \|CIB1 \|HPS4 \|DTX2 \|TRPV2 \|MAN2A1 \|KLF13 \|TBCK \|ANKFY1 | 4.817126839 | 0.28554 | 0.73933 | 0.15716 | −0.9608 | 0.92442 |
| PARP10 \|MYH11 \|MTHED2 \|RNF121 \|DPH5 \|METTL23 \|STX11 \|NAT9 \|MYC \|SIKE1 | 4.811199574 | 0.27207 | 0.89731 | 0.14725 | −0.226 | 0.92555 |
| ACTR1B \|BTG2 \|PDCL3 \|COG7 \|MRPS35 \|ZNF544 \|POP5 \|SUPT7L \|DHRS1 \|SORBS3 | 4.806472131 | 0.28807 | 0.8898 | 0.17454 | −0.5833 | 0.92456 |
| HPRT1 \|DVL3 \|MLC1 \|INPP5B \|UQCR10 \|NUP205 \|LANCL1 \|SUDS3 \|GOLIM4 \|RELT | 4.803179018 | 0.27098 | 0.90983 | 0.19186 | −0.5541 | 0.93343 |
| TMEM141 \|UBFD1 \|CAMKK2 \|MSH3 \|HPS3 \|UGCG \|RPL22L1 \|NSL1 \|UNC50 \|FBXO18 | 4.799543667 | 0.28151 | 0.87391 | 0.17473 | −1.0211 | 0.93924 |
| SCRN1 \|GFPT1 \|ATG4A \|BTBD9 \|PIGY \|PHF5A \|FURIN \|KIR3DL1 \|RP2 \|SHPRH | 4.796106574 | 0.26237 | 0.94987 | 0.10453 | −0.9858 | 0.93922 |
| SLC15A2 \|DOPEY2 \|SSH1 \|AP2S1 \|EVI5 \|UNC93B1 \|PREP \|HECTD3 \|CYP4V2 \|MON1B | 4.792414106 | 0.25151 | 0.92871 | 0.11845 | −0.1502 | 0.93033 |
| TRIM66 \|TRMT10B \|RNF41 \|GCH1 \|C10orf76 \|SLC39A13 \|DDI2 \|ACACB \|PPP1R18 \|SLC38A9 | 4.786068409 | 0.26839 | 0.9403 | 0.10752 | 0.17989 | 0.93073 |
| CPT1B \|RGL2 \|MOB2 \|PRKCA \|UBXN6 \|DENND1B \|COPS5 \|MAGED2 \|HADH \|CYTH2 | 4.780291585 | 0.25574 | 0.92081 | 0.11701 | 0.24468 | 0.91755 |
| YPEL2 \|KIAA0586 \|APBB3 \|PSMB2 \|FAM208B \|GGT1 \|PBX3 \|ABCC10 \|PFKFB4 \|ZNF429 | 4.775219895 | 0.28851 | 0.84991 | 0.11384 | −0.4448 | 0.91805 |
| ZNF862 \|AK3 \|GP6 \|SPICE1 \|UBN2 \|SLC16A6 \|BRIX1 \|HCCS \|RASSF2 \|ZMYM5 | 4.771115657 | 0.27446 | 0.93875 | 0.01335 | 0.61 | 0.92225 |
| SUCLA2 \|WDR82 \|DIS3L \|PIK3CA \|PITPNM1 \|NHLRC3 \|LPCAT4 \|SH3GLB2 \|INVS \|FLVCR1 | 4.766520404 | 0.28081 | 0.70863 | 0.20356 | −0.7391 | 0.91878 |
| AASDH \|IRF5 \|ATL2 \|THEM4 \|ERLEC1 \|IFNAR1 \|LACTB \|GFM2 \|MTA3 \|CRKL | 4.761053385 | 0.26534 | 0.76088 | 0.25381 | −0.8851 | 0.93534 |
| PFDN4 \|ADAM8 \|MIF \|PTGS2 \|TSNAX \|CLMN \|EDEM3 \|PTP4A1 \|ACTA2 \|ZNF789 | 4.758227228 | 0.29103 | 0.77707 | 0.25166 | 0.12945 | 0.92658 |
| FCGBP \|NOL11 \|FNIP1 \|SLC12A4 \|IQCG \|AHCYL2 \|MTOR \|TBC1D3F \|AKAP1 \|ICAM2 | 4.754027222 | 0.27418 | 0.79505 | 0.12787 | −0.7358 | 0.92523 |
| STARD3NL \|PF4V1 \|MFSD12 \|MAEA \|SLC16A3 \|RIPK1 \|P2RX4 \|SLC39A6 \|ZNHIT3 \|MRPS15 | 4.750591439 | 0.27587 | 0.82125 | 0.14524 | −0.2163 | 0.92337 |
| PGM1 \|B4GALT3 \|CARS \|MANF \|VKORC1 \|LTBP4 \|BCL2L13 \|MVK \|GGCX \|GOLT1B | 4.747235346 | 0.27782 | 0.8252 | 0.23719 | −0.5015 | 0.9304 |
| DNM3 \|ACCS \|ZNF586 \|COMMD3 \|MRFAP1 \|FOXO1 \|TERF1 \|GTPBP4 \|ETV6 \|XPNPEP3 | 4.741812634 | 0.28446 | 0.81811 | 0.17141 | −0.7196 | 0.9283 |
| CDC42EP3 \|ASPH \|ADAM19 \|EIF1B \|IDE \|MAP1LC3B \|TMEM127 \|DCTN3 \|SESN3 \|ICAM1 | 4.736519994 | 0.29879 | 0.80815 | 0.07752 | −0.9862 | 0.91923 |
| FBXW5 \|RAB9A \|TIPARP \|BRCC3 \|OCIAD2 \|PPA2 \|SEC24A \|DHX30 \|APOBEC3F \|CWC25 | 4.733254783 | 0.28481 | 0.85531 | 0.17091 | −0.4546 | 0.92387 |
| FAM160A2 \|RNF170 \|ODF2L \|HEXDC \|CRELD1 \|RASAL3 \|NEIC \|CYP20A1 \|LSM4 \|SESN1 | 4.726912285 | 0.28917 | 0.62063 | 0.20209 | 0.34294 | 0.92351 |
| TAF7 \|TMEM30A \|NAPG \|TMEM184C \|STAC3 \|DPP4 \|COPS7B \|PTER \|B4GALT1 \|ANO10 | 4.721994961 | 0.27071 | 0.89156 | 0.11888 | −0.7696 | 0.93652 |
| SYNJ1 \|INCENP \|ZNF326 \|CCDC137 \|CSE1L \|SSEP4 \|HGSNAT \|MRPS34 \|EBNA1BP2 \|FAM156B | 4.718899421 | 0.3024 | 0.58294 | 0.2731 | 0.06286 | 0.92561 |
| ASF1A \|ZNEX1 \|SNX11 \|KIAA1191 \|JMEM184B \|MGAT4A \|NSMCE1 \|CCDC88B \|SEXN4 \|DDX58 | 4.715845554 | 0.27913 | 0.97661 | 0.20538 | −0.6469 | 0.93008 |
| CNOT10 \|DOPEY1 \|TRABD \|ERH \|GLA \|SMNDC1 \|ROBO3 \|ZBTB33 \|PACRGL \|TMEM106A | 4.710502456 | 0.25821 | 0.83963 | 0.11679 | −0.4939 | 0.93857 |
| PTK2 \|PALLD \|ALDH5A1 \|EDC3 \|AQP3 \|CLK4 \|CDC34 \|MRPS11 \|ATAD2B \|HINFP | 4.704920218 | 0.25999 | 0.75903 | 0.15612 | 0.27184 | 0.93143 |
| LPGAT1 \|CNPPD1 \|COX15 \|NPHP3 \|ST6GALNAC6 \|SFT2D1 \|NOC2L \|AK1 \|MYO9A \|PTGDR | 4.698748749 | 0.29259 | 0.69189 | 0.22608 | −0.1633 | 0.91823 |
| GTF2A2 \|SLFN13 \|UCHL3 \|GNE \|ASB3 \|PDZD8 \|ZNF814 \|ROCK2 \|NFYA \|NHSL2 | 4.694458609 | 0.26541 | 0.88767 | 0.1474 | −0.418 | 0.92995 |
| HINT2 \|H2AFZ \|KDELR1 \|SULT1A4 \|RPS6KC1 \|TBC1D8 \|SIRT7 \|OTUB1 \|NAB1 \|BNC2 | 4.690983205 | 0.28878 | 0.74675 | 0.20393 | −0.4004 | 0.91364 |
| EMC4 \|ERP44 \|FUZ \|MAP3K7 \|KIF13A \|TBX21 \|POMZP3 \|GLYCTK \|RNASEH2A \|UBN1 | 4.685951036 | 0.27188 | 0.75055 | 0.17379 | −0.2342 | 0.93366 |
| DHRS12 \|CEP78 \|CSPP1 \|MTMR12 \|MRPL19 \|SNAPC3 \|RCBTB2 \|RNF44 \|CTDP1 \|CREBZF | 4.681374609 | 0.29009 | 0.77547 | 0.21598 | −0.7822 | 0.93132 |
| PCGF5 \|HIVEP2 \|CTLA4 \|ZNE805 \|CD99L2 \|JARID2 \|HIBADE \|GTF2IRD2 \|THTPA \|TRAPPC6B | 4.673391209 | 0.27534 | 0.89806 | 0.12219 | −0.6533 | 0.93066 |
| PPP2R2B \|POMGNT1 \|RIPK3 \|CSRP1 \|HTRA2 \|CCDC124 \|ADAMTS10 \|RAB1A \|TYW3 \|HMGXB3 | 4.667151847 | 0.22773 | 1.15491 | 0.02267 | −0.4931 | 0.92679 |
| P4HA1 \|SEC16A \|RTCA \|COA5 \|LEPROT \|AVL9 \|NOB1 \|PIBF1 \|CEBPZ \|PPDZD4 | 4.661926521 | 0.27005 | 0.60347 | 0.22959 | −0.7688 | 0.93515 |
| KHNYN1 \|IFT43 \|JOSD1 \|CD27 \|RNF146 \|HSF4 \|RBBP8 \|H2AFJ \|ALG2 \|PLGLB1 | 4.656811553 | 0.2612 | 0.92832 | 0.10045 | −0.9955 | 0.93293 |
| DLAT \|CACNA2D3 \|B3GALNT2 \|CTNS \|PPP1R12B \|NIT1 \|MYO1C \|MICU1 \|NUDT4 \|SH2D2A | 4.648990895 | 0.26581 | 0.82567 | 0.07113 | −0.2693 | 0.91675 |
| SEXN1 \|SLC39A8 \|SLC8A1 \|CYBSR1 \|VARS \|RREB1 \|BRPF3 \|PUF60 \|MICB \|FBXL20 | 4.643045318 | 0.27341 | 1.05082 | 0.07701 | −0.6697 | 0.92433 |
| MRPL11 \|WDR91 \|VTI1B \|RASGEF1B \|GNL1 \|C19orf12 \|FKBP4 \|CUL2 \|HIST1H2BH \|ATP6V1H | 4.636159107 | 0.27134 | 0.82341 | 0.1186 | −0.7428 | 0.93163 |
| RBM14 \|WIPF2 \|NPRL2 \|TXNDC9 \|SP4 \|ZNF738 \|SH2B3 \|RNF14 \|UBL7 \|CDKL5 | 4.630172711 | 0.29956 | 0.70549 | 0.22593 | −1.0122 | 0.92945 |
| ZNF600 \|TIMMDC1 \|POLD3 \|IKZES \|ALG12 \|TUBB6 \|HTATSF1 \|TRIB1 \|RABGEF1 \|BRMS1 | 4.62690416 | 0.28745 | 0.58671 | 0.25365 | 0.87822 | 0.92397 |
| CLOCK \|RSBN1L \|RNF6 \|TRMT13 \|ITSN1 \|KCTD18 \|TRRAP \|PTPRM \|TRAPPC4 \|U2AF1 | 4.621007103 | 0.27848 | 0.82953 | 0.12123 | −0.9188 | 0.93491 |
| TERF2IP \|PTPN1 \|IL12RB2 \|NKIRAS2 \|PCIF1 \|CRCP \|HIST1H3H \|C19orf66 \|TSPAN31 \|MKS1 | 4.616974122 | 0.28472 | 0.92932 | 0.11325 | 0.14013 | 0.93222 |
| DEDD \|JM6SF1 \|SNRPB \|ZFP62 \|TSPAN2 \|TMOD2 \|KLF11 \|ULK3 \|KAT8 \|TMEM62 | 4.610441438 | 0.2819 | 0.70784 | 0.199 | 0.40344 | 0.92177 |
| GRINA \|HINTS9 \|PRAGB \|EPB41L2 \|TMEM230 \|DOK1 \|MNAT1 \|RIPK2 \|TMEM131 \|AGBL5 | 4.606388572 | 0.28869 | 0.78002 | 0.21777 | −0.8903 | 0.9289 |
| SNRPE \|APOA2 \|USP30 \|GOLGA6L10 \|DYRK4 \|THAP6 \|RBMS2 \|C5orf42 \|WSB2 \|P2RY12 | 4.602518859 | 0.2482 | 0.86679 | 0.09708 | −0.5835 | 0.93413 |
| MRPS9 \|THEMIS \|TRIM4 \|NOP14 \|SSSCA1 \|GNB4 \|PIN1 \|VRK2 \|DERA \|LRCH1 | 4.595960925 | 0.27596 | 0.8569 | 0.12028 | −1.0078 | 0.94224 |
| PIGL \|RRP1 \|KIF1C \|FKBP11 \|QDPR \|GTF2H1 \|UBE3B \|COPS8 \|VPS45 \|BCKDHA | 4.589033058 | 0.27479 | 0.91894 | 0.12887 | −0.8025 | 0.93487 |
| FADS1 \|KIF21B \|POLR2M \|PATL1 \|MRPL24 \|C6orf203 \|FPR2 \|EVI2A \|AGFG2 \|FOPNL | 4.581574463 | 0.28166 | 0.71091 | 0.17489 | −0.0465 | 0.93554 |
| CLEC1B \|ZNF385A \|CHIC2 \|ACSL1 \|TREML1 \|SMYD2 \|C16orf70 \|PPM1D \|FOXO3 \|CUL5 | 4.572414059 | 0.26153 | 0.97566 | 0.04258 | −0.1451 | 0.92877 |
| KLHL12 \|MUC20 \|CFB \|HSD17B7 \|TRIM8 \|PDE5A \|COMMD7 \|GLTP \|UBE2D1 \|ARAGAP5 | 4.567799559 | 0.25654 | 0.97424 | 0.13345 | −0.5808 | 0.92558 |
| UHMK1 \|JDRD3 \|USP20 \|TNKS2 \|TSPO \|EDEM2 \|JAM3 \|MFSD11 \|GHRL \|NCAM1 | 4.56298483 | 0.27 | 0.9984 | 0.13528 | −0.5677 | 0.93341 |
| D2HGDH \|PLEKHF2 \|U2AF1L4 \|ZNF69 \|MRPL32 \|FBRS \|NDUFAF6 \|HIVEP1 \|SMPD1 \|RPS6KA4 | 4.556876751 | 0.29048 | 0.60106 | 0.27093 | −0.7635 | 0.9133 |
| PIAS2 \|C5orf22 \|ALG3 \|ANK3 \|RNF5 \|PRKCE \|TRMT11 \|LTB4R \|PREB \|THAP5 | 4.550681829 | 0.27369 | 0.87661 | 0.15821 | 1.58254 | 0.93746 |
| GNAQ \|ODF3B \|FAM118A \|RRP36 \|EXOC6B \|CPQ \|EIF1AD \|ERCC4 \|METTL5 \|ARPC4-TTLL3 | 4.545474604 | 0.28698 | 1.02248 | 0.19246 | −0.8481 | 0.92411 |
| TUBGCP3 \|FHIT \|RNPEPL1 \|PFDN2 \|SLAMF1 \|LZIC \|MRPL54 \|ZNF85 \|ABI3 \|ZNF548 | 4.53744865 | 0.27389 | 0.95342 | 0.12345 | −0.4525 | 0.93109 |
| S1PR4 \|RALGAPA1 \|CPOX \|EXTL3 \|TMEM50B \|CCL3L3 \|ZNF680 \|RPIA \|SGPL1 \|TDG | 4.533873624 | 0.2782 | 0.79327 | 0.16203 | −0.2371 | 0.92823 |
| FAM228B \|CD19 \|BLVRB \|LYSMD3 \|GIMAP8 \|RFX7 \|FAN1 \|COQ2 \|KIAA1328 \|SLC7A6OS | 4.529501259 | 0.26686 | 0.69213 | 0.20966 | 0.50261 | 0.92922 |
| COL18A1 \|ARG1 \|PAPD5 \|MTF1 \|AGAP1 \|ELOF1 \|PRKAB1 \|CHPT1 \|ATE1 \|BYSL | 4.525338766 | 0.25575 | 0.82621 | 0.17873 | 0.4101 | 0.92529 |
| RANGRE \|SRA1 \|KIF3A \|ZNF587B \|MRPL47 \|TBC1D17 \|BCKDHB \|CPSF1 \|ZNF701 \|R3HCC1L | 4.522807306 | 0.28817 | 0.8815 | 0.10324 | −0.7279 | 0.93243 |
| NEK6 \|RAB11FIP3 \|AGL \|SLC29A3 \|REST \|SKP2 \|SETD1B \|FBXL17 \|CERKL \|DCAF15 | 4.51941279 | 0.28358 | 0.68015 | 0.21436 | −0.6921 | 0.91534 |
| FCRL1 \|ASB1 \|TRANK1 \|NRF1 \|SMPD2 \|GCDH \|TNNI2 \|ZFP64 \|PANK4 \|BAG5 | 4.514421654 | 0.26267 | 0.8836 | 0.15749 | −0.5963 | 0.91009 |
| NUP43 \|UBE2J1 \|ARHGAP11B \|NIPSNAP3A \|SCO1 \|MIF4GD \|THAP1 \|MED29 \|POLRMT \|POLE4 | 4.509148597 | 0.29734 | 0.75459 | 0.26683 | −0.6665 | 0.93187 |
| BTBD10 \|IL1RN \|OGFRL1 \|TATDN1 \|GM2A \|PASK \|HLA-DOB \|ZMYM6 \|MSH2 \|RUVBL1 | 4.505112653 | 0.27946 | 0.72957 | 0.21075 | −0.3472 | 0.93621 |
| BCAP29 \|SIRPB2 \|SLC4A1AP \|CTAGE4 \|TSSK4 \|DNAJB2 \|MERTK \|TMX1 \|TMEM70 \|ZFAT | 4.500714141 | 0.27338 | 0.90142 | 0.12539 | −0.2904 | 0.93767 |
| BCL10 \|AGAP3 \|ANKRD20A1 \|SCAMP3 \|SLC30A5 \|N4BP2L1 \|VPS26B \|MED10 \|TPK1 \|FAAH | 4.49538775 | 0.27828 | 0.88469 | 0.06934 | −0.3235 | 0.92148 |
| CD300LB \|PLEKHG3 \|PAFAB2 \|USP14 \|TTC39B \|VPS25 \|RBM12B \|ANKRD13C \|DCUN1D2 \|POLR1B | 4.490151032 | 0.28154 | 0.80872 | 0.11573 | 0.19532 | 0.93189 |
| NEK9 \|GCNT2 \|EFEMP2 \|PTGS1 \|COX19 \|ADAT1 \|BZW2 \|SLC25A20 \|KLHL20 \|EMG1 | 4.4840107 | 0.2865 | 0.86813 | 0.23557 | −0.4517 | 0.92874 |
| PELI1 \|HHEX \|NTRK1 \|MRPL1 \|TATDN3 \|HRH2 \|RPS10-NUDT3 \|ACBD4 \|RAB4A \|CDC5L | 4.480540931 | 0.27998 | 0.89898 | 0.16481 | 0.03766 | 0.92853 |
| MAP3K11 \|RNFT1 \|TMEM116 \|PIGB \|MEI1 \|SCFD2 \|ZNF836 \|RPAP2 \|UBAP1 \|PIGS | 4.476650989 | 0.28825 | 0.86622 | 0.16612 | 0.0142 | 0.9291 |
| CDC42SE2 \|MCM4 \|DERL2 \|C1orf228 \|ARL6IP6 \|EPM2AIP1 \|PIM1 \|MTMR2 \|PAAF1 \|MRPL21 | 4.472352203 | 0.3008 | 0.67281 | 0.1518 | −0.7482 | 0.937 |
| UPF1 \|MNT \|TSEN34 \|ACBD3 \|IKZF2 \|AASDHPPT \|PGS1 \|PPP2R1B \|MGLL \|SORT1 | 4.467011556 | 0.29286 | 0.77984 | 0.24079 | −0.9596 | 0.93052 |
| UBE2L3 \|MAP2K6 \|GTF3C3 \|METTL16 \|NIPAL2 \|RUVBL2 \|FAM53B \|UFSP2 \|ZNF195 \|RIN2 | 4.463395767 | 0.26938 | 0.88887 | 0.19469 | −0.8434 | 0.93557 |
| DDX20 \|MAP3K5 \|NR3C2 \|ATP5D \|CRISPLD2 \|MORC2 \|MYLIP \|EIF2AK4 \|MTFR1 \|NT5E | 4.459435597 | 0.28694 | 0.81999 | 0.14966 | −0.7329 | 0.93301 |
| KLF4 \|SAMM50 \|RPRD1B \|NCAPH2 \|AP3M2 \|PYROXD1 \|MCRS1 \|TET3 \|WDFY4 \|MDN1 | 4.454370731 | 0.25137 | 0.93522 | 0.06643 | −0.2355 | 0.93033 |
| TMEM206 \|PRPSAP1 \|CNIH4 \|SETD4 \|GMFB \|CBFA2T2 \|SLC38A10 \|POLH \|RAD54L \|TMEM156 | 4.449241491 | 0.26305 | 0.8799 | 0.16178 | 0.15028 | 0.93579 |
| NIT2 \|RBMX2 \|C11orf49 \|GDAP1 \|NQO2 \|SETD7 \|CHMP1A \|MFNG \|C1orf174 \|KCTD2 | 4.443158727 | 0.27629 | 0.78498 | 0.11987 | −0.759 | 0.91767 |
| KCNE3 \|TNFRSF10C \|ZNF286A \|CHTOP \|CD40LG \|LAMB1 \|GADD45B \|VAC14 \|NFKB2 \|SMG9 | 4.439729881 | 0.26973 | 0.97417 | 0.07224 | −0.8548 | 0.92472 |
| PLEKHB2 \|ARL15 \|NAA35 \|SPATA20 \|MAP7D3 \|JAKMIP2 \|COX17 \|C12orf65 \|ZNF33B \|PEF1 | 4.436408669 | 0.27761 | 0.90535 | 0.07655 | −0.0181 | 0.93516 |
| PCGF3 \|APOPT1 \|DPH1 \|RBM18 \|MYLK \|NFYB \|CDC23 \|FAM185A \|ARFIP1 \|POFUT2 | 4.433329765 | 0.28222 | 0.67441 | 0.26517 | −0.9396 | 0.92548 |
| MYO19 \|TLR6 \|SGMS2 \|CYB5B \|RPS6KA5 \|BATF \|DDHD1 \|FAM53C \|TUBGCP6 \|ATMIN | 4.429382169 | 0.28419 | 1.00588 | 0.10755 | −0.5149 | 0.93491 |
| SDCCAG3 \|HPS6 \|TAF1B \|CPT2 \|RAB27B \|NOTCH1 \|MAPK9 \|CCDC90B \|PDE6B \|OR2A1 | 4.424625693 | 0.24359 | 0.93696 | 0.16315 | −0.0524 | 0.9182 |
| INTS7 \|DST \|LTN1 \|NUBPL \|TMEM168 \|REXO2 \|CMTM7 \|COA1 \|COBLL1 \|MRPS33 | 4.418143926 | 0.26266 | 0.94599 | 0.08354 | −0.23 | 0.94795 |
| ST7L \|CRYZ \|UBLCP1 \|DUS1L \|CCDC126 \|ELAVL1 \|ARHGAP35 \|KLHEL8 \|WRN \|LAS1L | 4.413564864 | 0.27711 | 0.95898 | 0.08326 | 1.31432 | 0.9343 |
| OR2A42 \|NSMCE4A \|APOBEC3D \|SH3TC1 \|PUS7L \|TPMT \|UBASH3B \|C12orf29 \|EHBP1 \|C19orf24 | 4.409067196 | 0.28454 | 0.8014 | 0.18584 | −0.5325 | 0.92388 |
| GPATCH4 \|PACS2 \|RNF139 \|NFATC2IP \|MRPS31 \|DBT \|ZNF235 \|APPBP2 \|KRAS \|ZNF552 | 4.403235351 | 0.27957 | 0.62213 | 0.25574 | −0.5856 | 0.93695 |
| SLC36A4 \|UBE2M \|DNAJC3 \|PKNOX1 \|MYOF \|ETFDB \|ZMIZ1 \|TRMT6 \|TNS3 \|SLC12A7 | 4.396516639 | 0.26777 | 0.89674 | 0.0192 | −0.9372 | 0.9195 |
| FN1 \|CATSPERG \|TUBGCP5 \|ARID5B \|RNF141 \|MIB2 \|CXorf40B \|HPSE \|GDPD5 \|SNUPN | 4.392062165 | 0.26762 | 0.82693 | 0.22166 | 0.23766 | 0.92888 |
| DMAP1 \|DPP3 \|C1orf123 \|C5orf24 \|P2RX1 \|CHMP6 \|R3HCC1 \|FAH \|TLR2 \|RPF2 | 4.387934607 | 0.27982 | 0.75534 | 0.14787 | −0.1875 | 0.92892 |
| PSMC3IP \|TRIM41 \|CISD2 \|COQ6 \|C15orf39 \|LIMK1 \|AMBRA1 \|FAM162A \|KDM4B \|ATG9A | 4.381872412 | 0.27624 | 0.78657 | 0.18285 | −0.5464 | 0.92233 |
| PRMT3 \|SLC50A1 \|CD7 \|DNAJC4 \|SNX12 \|RNF25 \|RABL3 \|FAM160B1 \|FUCA1 \|SUMO3 | 4.376968729 | 0.28626 | 0.81829 | 0.0915 | −0.581 | 0.91037 |
| FLNB \|TSPAN3 \|ALDH18A1 \|CCDC6 \|ZNF816 \|HAUS2 \|SIK2 \|SLC12A9 \|DPY19L3 \|MAF | 4.371233154 | 0.26633 | 0.86934 | 0.18413 | −0.6316 | 0.92323 |
| COG2 \|ZNF321P \|VILL \|ZBTB7B \|CNEP1R1 \|RRNAD1 \|CPM \|STX2 \|PLD1 \|KPNA4 | 4.366677828 | 0.2635 | 0.85828 | 0.06106 | 0.59051 | 0.93097 |
| ZDHHC8 \|ZDHHC13 \|SF3B4 \|PRKCI \|THRA \|ERLIN1 \|ORC6 \|CATSPER1 \|ALDH9A1 \|SIAE | 4.360575091 | 0.25889 | 0.93048 | 0.14756 | 0.02786 | 0.92776 |
| CMAS \|RNF135 \|TUBGCP2 \|SMG6 \|RAD51D \|DESI1 \|IL15RA \|GTF2H3 \|PPCDC \|LAMP1 | 4.354126282 | 0.26903 | 0.87542 | 0.18459 | −0.3203 | 0.92154 |
| ZNHIT1 \|PRDX3 \|OSBPL11 \|DUSP12 \|CRLS1 \|UBASH3A \|POGLUT1 \|TEX2 \|TSC22D1 \|FAM129C | 4.342930973 | 0.2851 | 0.98923 | 0.11054 | −0.7872 | 0.92981 |
| ALPK1 \|CEP128 \|TTC4 \|GLMN \|FH \|RASGRF2 \|HSF2 \|LAMTOR3 \|SLC4A2 \|TCP11L1 | 4.336195755 | 0.27031 | 0.69419 | 0.255 | −0.9909 | 0.93956 |
| LARS2 \|ATAD1 \|MZT2B \|C2orf88 \|POLR2L \|F8A3 \|TTYH3 \|VARS2 \|FAM105A \|FGFRIOP | 4.33210591 | 0.27414 | 0.66271 | 0.26337 | −0.827\| | 0.91375 |
| CUL9 \|GLI4 \|DNASE2 \|GAPT \|POLM \|HELB \|PLEKHA1 \|RNF43 \|CDKAL1 \|TM2D3 | 4.327174489 | 0.27537 | 0.72478 | 0.15981 | −0.9588 | 0.94287 |
| KCNJ13 \|KPNA2 \|YIF1A \|RIC3 \|CNP \|ITPR3 \|PPP1R35 \|LYPLAL1 \|CTTN \|IFFO1 | 4.3205283 | 0.2716 | 0.93911 | 0.09732 | 0.03391 | 0.93117 |
| CSAR1 \|KATNA1 \|QSER1 \|PHRF1 \|HEATR3 \|GALNT11 \|ZNE354A \|DLEC1 \|ZBTB17 \|MEA1 | 4.316392468 | 0.27022 | 0.79557 | 0.20062 | −0.6417 | 0.93113 |
| CCDC57 \|SLC35E1 \|ZDHHC16 \|C16orf72 \|USP40 \|MILR1 \|NR1D2 \|CMKLR1 \|CLDN15 \|ZNF780A | 4.310905212 | 0.25048 | 1.0793 | 0.07767 | −0.2851 | 0.9304 |
| ZNF397 \|SLC35D2 \|UFM1 \|TTF1 \|NDUFAF1 \|CDADC1 \|SEMA4B \|TMEM39A \|HAT1 \|TWF2 | 4.305894522 | 0.29375 | 0.71503 | 0.24963 | −0.5221 | 0.94058 |
| IGF2BP3 \|GUF1 \|CLCC1 \|MPP6 \|MRPL40 \|TRAK2 \|PPP1R16B \|CD72 \|CNNM3 \|NAE1 | 4.302092719 | 0.26018 | 0.94016 | 0.09278 | −0.7946 | 0.91662 |
| REM15 \|DR1 \|BUD13 \|TXNDC5 \|GPSM2 \|SETBP1 \|OSBPL5 \|NME1-NME2 \|DNAJC1 \|NDUFC1 | 4.29755336 | 0.2647 | 0.84665 | 0.12517 | −1.047 | 0.93627 |
| SHQ1 \|TMEM63B \|CTBP2 \|TARSL2 \|BRCA1 \|RABEP2 \|USP45 \|PLEKHG2 \|TNFSF12 \|CD200R1 | 4.294515123 | 0.26548 | 0.92307 | 0.16237 | 0.57608 | 0.93327 |
| DNHD1 \|NR1H2 \|TUBA8 \|ELP4 \|TMEM67 \|SPNS1 \|GPR171 \|TRAPPC2 \|HSPBAP1 \|TRAPPC3 | 4.291988387 | 0.25881 | 0.86428 | 0.16402 | 0.08227 | 0.93677 |
| TMEM14C \|ZFAND1 \|RRP7A \|KANSL1L \|SIN3B \|ERCC8 \|ZNF783 \|CSTF1 \|LSM10 \|MYBBP1A | 4.288681621 | 0.28624 | 0.69492 | 0.29203 | −0.769 | 0.93311 |
| PRDX4 \|TBC1D19 \|BTRC \|ZNF117 \|RMND1 \|C18orf21 \|MCM5 \|GMPPA \|H1F0 \|SUZ12 | 4.282964858 | 0.26706 | 0.75386 | 0.14512 | −1.0258 | 0.93879 |
| RPTOR \|EXOC2 \|EMC1 \|RCAN3 \|SLC25A40 \|NUDT9 \|HPS5 \|HACL1 \|ITIH4 \|ALAD | 4.278079655 | 0.26764 | 0.87891 | 0.13789 | −1.0278 | 0.9341 |
| TAF11 \|TMEM176A \|ZNE652 \|ZNF551 \|CASP6 \|AURKAIP1 \|HEBP2 \|DRG2 \|LCAT \|ANAPC10 | 4.27301259 | 0.27842 | 0.89356 | 0.19749 | −0.8229 | 0.91696 |
| NDUFA8 \|PTMS \|TRUB1 \|NEU1 \|REXO4 \|NT5C \|ZNF121 \|SLC36A1 \|PKP4 \|AXIN1 | 4.267482705 | 0.27575 | 0.7929 | 0.24437 | −0.7073 | 0.9297 |
| TOMM5 \|FBXW11 \|SLC16A7 \|NCAPD3 \|ARRDC5 \|UBE2J2 \|UTP3 \|NIF3L1 \|RPRD1A \|RSBN1 | 4.258270469 | 0.26641 | 0.79211 | 0.14648 | −0.7295 | 0.9379 |
| RNF19B \|POLE3 \|HAUS4 \|SLC25A11 \|CCDC134 \|ZNF621 \|TNPO2 \|CBR4 \|IDUA \|MEX3C | 4.252651956 | 0.28846 | 0.79369 | 0.22097 | −0.2955 | 0.9265 |
| SBNO2 \|IDH1 \|ATG4C \|GPR174 \|ALCAM \|SLC37A2 \|SLC2A9 \|JDP2 \|ARHGEF10L \|PGM3 | 4.247538582 | 0.23938 | 0.96914 | 0.12185 | −0.0493 | 0.91654 |
| UBE2R2 \|TBK1 \|ALYREF \|TIGIT \|YIPF1 \|IFFO2 \|TMED7 \|84GALT7 \|MTAP \|SETDB2 | 4.240022921 | 0.29206 | 0.85682 | 0.12447 | 0.09559 | 0.92766 |
| ZNF782 \|MASTL \|SPRTN \|CAMKMT \|RAB30 \|PB41L4A \|HIST1H3C \|PNPT1 \|HMGXB4 \|RABAC1 | 4.232301198 | 0.2733 | 0.74309 | 0.17063 | −0.8951 | 0.93291 |
| GABPB1 \|MICA \|HGF \|SEC31B \|CRTC1 \|USP37 \|ACRBP \|COL17A1 \|SLC17A5 \|CLPB | 4.225704798 | 0.27013 | 0.87645 | 0.20326 | 0.39526 | 0.93084 |
| PDCD2 \|TBL2 \|ARHGAP18 \|UBE2D4 \|DEDD2 \|TRAF3IP1 \|IMPACT \|BTF3L4 \|ANAPC16 \|ZC3H12A | 4.219400888 | 0.29283 | 0.75222 | 0.26611 | −0.0257 | 0.93354 |
| ANKHD1-EIF4EBP3 \|TMLHE \|MRPL37 \|RNF103-CHMP3 \|GRK5 \|METTL13 \|WDR77 \|TARBP1 \|SGTA \|MRPL4 | 4.214959483 | 0.30382 | 0.84822 | 0.13603 | −0.0152 | 0.93214 |
| ENO3 \|C5orf58 \|GRASP \|ST3GAL4 \|ACVR1 \|STUB1 \|STK40 \|SLC25A16 \|EIF2B3 \|DCPS | 4.211155893 | 0.25478 | 0.94978 | 0.08139 | −0.3827 | 0.91474 |
| GTF3C6 \|EIF3CL \|ZNF317 \|XRCC1 \|APBB1 \|SLC15A4 \|LMTK2 \|POLR3D \|CYP2R1 \|IGFLR1 | 4.203341902 | 0.28323 | 0.89681 | 0.06885 | −0.0088 | 0.93 |
| C1orf54 \|USP42 \|ZNF318 \|TMCC1 \|ARF6 \|CRY2 \|IMPA1 \|MXD1 \|CATSPER2 \|INPP5A | 4.197854946 | 0.27424 | 0.84573 | 0.09346 | 0.6537 | 0.92853 |
| AMDHD2 \|MRPL45 \|ANKRD26 \|CANT1 \|ORC3 \|C3orf62 \|LIMK2 \|ULK4 \|SEPSECS \|LAX1 | 4.191824076 | 0.26559 | 0.83825 | 0.09586 | −1.0719 | 0.93076 |
| DNMT3A \|SEC22A \|ABCB4 \|GMEB1 \|PEX6 \|PIK3C2A \|PID1 \|ATG12 \|KLHL22 \|PPIF | 4.183706649 | 0.24955 | 0.83266 | 0.29166 | 0.19843 | 0.92906 |
| KCTD17 \|TMEM110-MUSTN1 \|POC5 \|AGAP2 \|ZRANB1 \|ORAOV1 \|CINP \|ESF1 \|C10orf128 \|FAM118B | 4.17757322 | 0.26577 | 0.9284 | −0.0317 | −0.5211 | 0.93083 |
| ZNF714 \|ALG11 \|OAS3 \|NAGPA \|PCSK5 \|AQP9 \|SIRPD \|KBTBD2 \|CDIPT \|ZFP90 | 4.17101404 | 0.25724 | 0.961 | 0.105 | −0.6652 | 0.93443 |
| ABHD3 \|FLCN \|SIVA1 \|FBXO42 \|ATRN \|FAM76A \|GOLGA6B \|BRAP \|SLC10A7 \|HIST1H4A | 4.164231322 | 0.29571 | 0.63067 | 0.21168 | −0.7226 | 0.92353 |
| KCNA3 \|ZNF791 \|ST7 \|NEURL4 \|DBF4 \|PHPT1 \|MYH3 \|ST20-MTHFS \|CREB3L2 \|TMA16 | 4.160061692 | 0.27128 | 1.02794 | 0.02317 | −0.226 | 0.92483 |
| TAS2R30 \|FUT8 \|ZBTB37 \|NOA1 \|CHAMP1 \|WDPCP \|PINX1 \|PARP2 \|METTL8 \|TADA3 | 4.154023094 | 0.28243 | 0.89827 | 0.14067 | −0.1307 | 0.94383 |
| TMEM126A \|TPCN2 \|MTHFSD \|TUBG1 \|PRR11 \|FBXO21 \|CGGBP1 \|CUEDC2 \|FAM168B \|GPR137B | 4.1505153 | 0.28141 | 0.60195 | 0.23777 | −0.7422 | 0.93352 |
| POT1 \|EEPD1 \|EIF6 \|TJAP1 \|FAAB2 \|FANCG \|ABCA1 \|DTX4 \|YTHDF2 \|OSBPL1A | 4.145354804 | 0.27776 | 0.78764 | 0.10517 | −0.0394 | 0.93198 |
| MTMR11 \|CDC26 \|ARHGEF12 \|VBP1 \|SUFU \|DGCR8 \|ZNF532 \|LCORL \|KIAA0753 \|TP53I13 | 4.138040963 | 0.27269 | 0.8398 | 0.13256 | −0.8617 | 0.92777 |
| IPO11 \|NDUFAF4 \|NDST1 \|MYEF2 \|PAX5 \|NCOA5 \|ZNF107 \|KCTD9 \|SETMAR \|ATPAF1 | 4.132064381 | 0.22619 | 0.92345 | 0.13389 | −0.5801 | 0.93746 |
| RTTN \|TLR5 \|SIGMAR1 \|MAP4K3 \|PHF7 \|TMEM189 \|TIMM9 \|ZNF740 \|CLN6 \|ACAA2 | 4.127371457 | 0.2856 | 0.65423 | 0.22798 | −0.477 | 0.92883 |
| ZBED5 \|PIGX \|CCDC141 \|WLS \|ZNF525 \|WDTC1 \|DAB2 \|PRPF4 \|DUSP2 \|ACVR2A | 4.11872572 | 0.27615 | 0.81216 | 0.14115 | −0.4489 | 0.9314 |
| ZNF440 \|SREK1IP1 \|TRAF6 \|MOB1B \|ATP8B4 \|MICAL3 \|MCEE \|CDK6 \|POLR3B \|CEP135 | 4.111058004 | 0.2758 | 0.89628 | 0.14547 | −0.8327 | 0.93664 |
| PCYOX1 \|NEIL3 \|SLC23A2 \|YEATS2 \|SLC25A22 \|MRPL49 \|SDR39U1 \|PTGER2 \|CBFA2T3 \|FNTB | 4.105437172 | 0.2732 | 0.85316 | 0.22425 | −0.4489 | 0.92504 |
| COL10A1 \|HEMK1 \|ARMC2 \|ASGR2 \|TTF2 \|MSRA \|LONP1 \|ZFAND4 \|ZNF75A \|MFAP3 | 4.099989522 | 0.27336 | 0.97433 | 0.14839 | −1.0969 | 0.94003 |
| IL2RA \|ZNF181 \|WDR83OS \|THOC6 \|C1orf21 \|IL18R1 \|DPCD \|MAGED1 \|EXOSC7 \|REXO1 | 4.094849796 | 0.25117 | 0.9929 | 0.06408 | 0.19332 | 0.92538 |
| GORAB \|GSTM1 \|TXNL4A \|CEMP1B \|MRPL51 \|GALM \|DOT1L \|PFDN6 \|SYS1 \|ZER1 | 4.08970591 | 0.29578 | 0.82884 | 0.20614 | −0.8974 | 0.9336 |
| USPL1 \|C1orf27 \|HAUS7 \|SKIL \|VAMP4 \|PSMD5 \|CETN3 \|HAUS3 \|EME2 \|ZBTB11 | 4.08449412 | 0.28968 | 0.70249 | 0.07111 | −0.5123 | 0.93693 |
| LMF1 \|JMJD6 \|TRADD \|UBIAD1 \|TRERF1 \|SLC38A6 \|OSGIN2 \|TRIM39 \|MEPCE \|IPO4 | 4.079245889 | 0.30346 | 0.75895 | 0.09326 | −0.2857 | 0.92029 |
| ZNF93 \|C15orf40 \|PLIN3 \|WDR55 \|NDRG3 \|ZCCHC9 \|TOR1B \|ATG10 \|PCED1B \|RTN1 | 4.074348007 | 0.29173 | 0.91571 | 0.08946 | −0.4446 | 0.92923 |
| C21orf91 \|SIRT1 \|CPSF4 \|MAD2L2 \|TMEM147 \|CCS \|CCND2 \|CROT \|PALB2 \|ZBTB24 | 4.070714167 | 0.26684 | 0.64399 | 0.2122 | −0.7629 | 0.91969 |
| CC2D2A \|ZNE654 \|SPEF2 \|RCE1 \|ZGPAT \|PMF1 \|DHX29 \|TBCCD1 \|STK36 \|ZNF281 | 4.066834566 | 0.26031 | 0.83434 | 0.16202 | −0.3855 | 0.93496 |
| CDK2 \|BLOC1S2 \|MBIP \|FBXO28 \|SURF2 \|PPP6C \|MBTD1 \|MAGOHB \|C11orf80 \|CHM | 4.062438464 | 0.27141 | 0.77386 | 0.26451 | −0.9526 | 0.93491 |
| CHAF1A \|ABI2 \|FMNL3 \|HEATR1 \|TUBE1 \|INO80C \|NIPSNAP1 \|RNF168 \|NDOR1 \|INSIG1 | 4.054570952 | 0.28559 | 0.62795 | 0.28571 | −0.8506 | 0.93531 |
| MRPS6 \|HDAC8 \|IDNK \|TRIM5 \|FAHD2A \|CNNM4 \|ZNF7 \|HCFC2 \|MAN2B2 \|PRKCZ | 4.048411867 | 0.28331 | 0.60859 | 0.21349 | −1.0336 | 0.91371 |
| EFCAB2 \|CMPK2 \|PTTG1 \|GLT1D1 \|GPX2 \|ASPSCR1 \|ESRP2 \|CLEC12B \|HGD \|CPED1 | 4.038941551 | 0.24249 | 1.10881 | 0.00998 | −0.4248 | 0.93412 |
| RETSAT \|MYH10 \|PERMP1 \|PCNA \|MAP3K12 \|ACTR6 \|ZBTB25 \|PLEKHF1 \|IER3IP1 \|BCKDK | 4.032847863 | 0.28754 | 0.9252 | 0.10346 | −0.6296 | 0.91795 |
| COMMD4 \|DSTN \|TBCEL \|CYP2D6 \|CLCN6 \|ACOX3 \|SAP130 \|MUM1 \|EEF2K \|TMEM181 | 4.030479162 | 0.24668 | 0.90495 | 0.11115 | −0.6792 | 0.91114 |
| KLHL28 \|FAM160B2 \|ZSWIM7 \|CDAN1 \|SLC2A1 \|PDIA5 \|TMEM167B \|KNTC1 \|ACTR8 \|SMAD1 | 4.025529339 | 0.28575 | 0.80672 | 0.18051 | −0.8797 | 0.92897 |
| SLC25A45 \|FKBP2 \|ASRGL1 \|CLEC4D \|AFMID \|GPS1 \|CARD9 \|SLC9A6 \|GRAMD4 \|AGPAT4 | 4.020278488 | 0.25036 | 1.10243 | 0.00475 | −0.5096 | 0.9154 |
| REPIN1 \|CFD \|INO80D \|DSE \|TOMM20 \|RALBP1 \|TAF9B \|C8orf76 \|GFI1 \|ALDH3B1 | 4.015152584 | 0.26715 | 1.09622 | 0.08571 | −0.5585 | 0.91816 |
| MRPL39 \|SPRYD3 \|ZCWPW1 \|ESCO1 \|FAM27B \|ENTPD5 \|MRPS30 \|SNX25 \|GCSH \|EXT1 | 4.012154364 | 0.28947 | 0.65227 | 0.22722 | 0.04809 | 0.93562 |
| C11orf65 \|RADS4L2 \|GNPNAT1 \|SLA2 \|ST8SIA4 \|R3HDM4 \|FGGY \|TUBG2 \|UTP14C \|MUC2 | 4.007752803 | 0.2336 | 1.05904 | 0.06434 | −0.6847 | 0.93035 |
| FAM159A \|DAGLB \|BMS1 \|RINL \|LOXL2 \|IFT57 \|MED8 \|ADCY3 \|ALS2CR12 \|PLA2G12A | 4.001866342 | 0.24753 | 0.88834 | 0.13865 | −0.4456 | 0.9263 |
| CRADD \|RNF7 \|MTX2 \|KMO \|DYRK2 \|SPATA5 \|RCN1 \|GENI \|NFE2L3 \|DDX31 | 3.996631815 | 0.26609 | 0.88548 | 0.14786 | −0.7609 | 0.93362 |
| RPS27L \|CCDC43 \|FAM168A \|SLC27A1 \|CAMK1 \|HAPLN3 \|PLA2G4A \|ZNF322 \|PTGES2 \|LEO1 | 3.991647671 | 0.27186 | 0.91752 | 0.1987 | −0.8046 | 0.9226 |
| TAF6 \|SPTY2D1 \|NDRG2 \|FRA10AC1 \|BACH2 \|PSMG1 \|DPY19L4 \|EOMES \|NDUFAB1 \|SLC22A15 | 3.987291302 | 0.26319 | 0.86247 | 0.05521 | 0.18654 | 0.92646 |
| SUSD3 \|KAT2A \|C1RL \|PRR12 \|ATP6V0C \|ACAP3 \|ARHGEF18 \|MBOAT7 \|IFIH1 \|NME1 | 3.979141431 | 0.28344 | 0.77597 | 0.19331 | −0.701 | 0.9107 |
| PITHD1 \|CAMSAP1 \|ZNF559 \|CRYGS \|RTEL1 \|MTRF1 \|ZNF813 \|BCL2L1 \|FAM120AOS \|CASD1 | 3.973739511 | 0.27599 | 0.85298 | 0.22195 | −0.5869 | 0.93602 |
| RASGRP4 \|CXorf40A \|RAP2C \|IER2 \|CHCHD7 \|ORMDL2 \|BNIP3 \|GOLGA6L1 \|GUCY1B3 \|NLN | 3.969134586 | 0.29327 | 0.61199 | 0.2803 | −0.3639 | 0.91856 |
| ATP10D \|TMEM164 \|ITPKB \|CEACAM21 \|SMYD3 \|WHAMM \|TCF3 \|HDAC10 \|LRIG1 \|CYP2S1 | 3.963334128 | 0.27222 | 0.86184 | 0.06966 | 1.76194 | 0.9151 |
| UNK \|C19orf53 \|AHCY \|CKAP4 \|TCTN3 \|NBPF3 \|ZNF257 \|ADAP1 \|DGAT1 \|CCDC71 | 3.956346821 | 0.28078 | 0.83514 | 0.17645 | −0.3504 | 0.91628 |
| LARP1B \|GNB5 \|KDM8 \|STRBP \|HPCAL1 \|RCC1 \|SNTB1 \|EMILIN2 \|TPD52L2 \|SERPINB9 | 3.951658718 | 0.27681 | 0.99839 | 0.11289 | 0.15155 | 0.91789 |
| L3MBTL2 \|DIRC2 \|TACO1 \|LZTFL1 \|TESK1 \|PPARA \|CHID1 \|XYLT1 \|NUDT22 \|GMCL1 | 3.946483405 | 0.28028 | 0.72636 | 0.25 | −0.2831 | 0.91452 |
| BOP1 \|MAP1LC3B2 \|RIC8B \|SHMT1 \|ACSF2 \|RLIM \|MIIP \|MEGF9 \|RBM41 \|PTGDS | 3.938802927 | 0.27518 | 0.78297 | 0.09973 | −0.4747 | 0.91358 |
| FARP2 \|KIAA0232 \|YIF1B \|CDC37L1 \|ZMYM1 \|GABPA \|XAB2 \|ZNF827 \|STMN1 \|ATP11C | 3.934281955 | 0.29249 | 0.8165 | 0.22473 | −0.2691 | 0.93065 |
| PPAN \|ZNF274 \|LRP8 \|PORCN \|QSOX1 \|EPHA4 \|MFAP1 \|MIOS \|COASY \|POLI | 3.927866423 | 0.29062 | 0.78999 | 0.22249 | −0.8631 | 0.92404 |
| SAMD8 \|SLC2A4RG \|RFC2 \|HAUS1 \|BCAS4 \|FASN \|RNMT \|AGPAT5 \|SDF2 \|YBEY | 3.923424492 | 0.28955 | 0.68985 | 0.21849 | −0.6823 | 0.92783 |
| ARHGEE5 \|FASTKD3 \|ADARB1 \|LMF2 \|CHMP2B \|MFSD10 \|ZNF550 \|CALHM2 \|C5orf15 \|TAC4 | 3.919623373 | 0.24537 | 0.89228 | 0.10893 | −0.7466 | 0.91232 |
| PRH1 \|ASPDH \|JIMM17B \|RPL26L1 \|TTI2 \|SERF1A \|TAB3 \|ZNF337 \|MCTS1 \|ZHX2 | 3.914102844 | 0.22517 | 1.24522 | 0.02296 | −0.9522 | 0.92822 |
| IGF2BP2 \|ZNF528 \|NAA40 \|MINOS1 \|TSFM \|TBC1D10B \|FXR2 \|MRPS18A \|NEDD4L \|ZC2HC1A | 3.90912226 | 0.27027 | 0.638 | 0.18491 | −0.9519 | 0.92387 |
| CHEK2 \|WDR12 \|WDR27 \|USP1 \|SBF1 \|TIAM1 \|H6PD \|ACD \|ZSWIM6 \|TMCO6 | 3.906243287 | 0.2889 | 0.6722 | 0.11132 | −0.4205 | 0.91425 |
| JAG1 \|C21orf33 \|GZMM \|RWDD4 \|LRIG2 \|ZNF227 \|TMCO3 \|RRAGA \|ZNF138 \|SPATS2L | 3.902301526 | 0.26526 | 0.78088 | 0.13118 | −0.3317 | 0.92364 |
| SNX15 \|ZNF830 \|ZNF45 \|MFSD5 \|ZNF169 \|PTPN9 \|SLC25A24 \|KIAA0355 \|TMEM91 \|NXT1 | 3.896129771 | 0.28523 | 0.85913 | 0.19953 | −0.3526 | 0.93096 |
| GMDS \|QTRT1 \|DOCK7 \|SPATA6 \|NIPAL3 \|TOP3A \|SMYD4 \|ERMN \|EBF1 \|PIGH | 3.892128712 | 0.26967 | 0.70012 | 0.24068 | −0.9046 | 0.93283 |
| GPR155 \|B3GNTL1 \|CEBPB \|TJP2 \|TUBGCP4 \|NPR2 \|KATNB1 \|PTRH2 \|SETD6 \|ZNF383 | 3.885754706 | 0.26418 | 0.84709 | 0.15764 | −0.8272 | 0.92721 |
| WDR5 \|TPRKB \|MBOAT1 \|ZNF846 \|NOM1 \|ABHD4 \|ATN1 \|ABCA5 \|EXOG \|TRAPPC6A | 3.880966009 | 0.2784 | 0.75327 | 0.25973 | 0.01865 | 0.92534 |
| LRRC47 \|CSTF3 \|UFL1 \|NEU3 \|PPP2R2D \|CCDC18 \|GRWD1 \|PINK1 \|NERKB \|HIP1 | 3.87552768 | 0.26343 | 0.90325 | 0.10041 | −0.7977 | 0.93716 |
| MEIS1 \|LIPN \|CCDC77 \|RXRB \|C11orf24 \|FAM135A \|CAD \|RINT1 \|TRMT61B \|WARS2 | 3.869605693 | 0.27203 | 0.78858 | 0.22107 | −0.5241 | 0.94643 |
| BTBD6 \|FNIP2 \|C4orf3 \|TMEM69 \|RBP5 \|FOCAD \|ZDHHC4 \|ZC3H7B \|ZXDC \|INTS1 | 3.86538759 | 0.27274 | 0.79519 | 0.11944 | −0.2864 | 0.9218 |
| MCOLN2 \|PLIN2 \|CCDC9 \|PRC1 \|ABCC5 \|XPR1 \|DUOX1 \|INIP \|SCAI \|TWISTNB | 3.859468843 | 0.25755 | 0.89253 | 0.10586 | 0.13327 | 0.92997 |
| ZNF710 \|RAD9A \|CEP290 \|APOL1 \|CUL7 \|TPD52 \|P4HTM \|CALML4 \|TMEM109 \|DFFB | 3.854228426 | 0.26732 | 1.00901 | 0.12873 | −0.5111 | 0.91529 |
| UCK1 \|HLA-DQB2 \|MKL2 \|ABCC3 \|KIFAP3 \|TTC5\|IL18BP \|ZNE567 \|IFT88 \|ARHGAP19 | 3.848843323 | 0.24953 | 0.89613 | 0.10583 | −0.2174 | 0.93438 |
| SLC25A51 \|ZNF354B \|RBL1 \|PRIM1 \|MED27 \|MRPL35 \|SNRNP25 \|PDCD11 \|NAGLU \|RCHY1 | 3.844178881 | 0.27588 | 0.91459 | 0.19766 | −0.5703 | 0.93586 |
| ZNF559-ZNF177 \|ZNF658 \|UGDH \|MTUS1 \|FAM78A \|AP4E1 \|VPS54 \|ITGAV \|TSPAN18 \|SLC5A6 | 3.839138797 | 0.26382 | 1.03333 | 0.03915 | −0.9147 | 0.93165 |
| SLC25A26 \|ZNF414 \|ZNRD1 \|ING5 \|ZNF263 \|TAF6L \|SLC39A7 \|POLD2 \|ZNF407 \|DNAJA4 | 3.834073133 | 0.27701 | 0.99124 | 0.06905 | 0.18467 | 0.93026 |
| BLM \|ALKBH6 \|GALK1 \|EPHA1 \|ECHS1 \|KLRC1 \|CARM1 \|TMEM57 \|XCL2 \|KCTD13 | 3.828328434 | 0.26222 | 0.94582 | 0.06408 | −0.3659 | 0.92587 |
| CAPS \|FOXJ2 \|HELQ \|DTHD1 \|HEXIM2 \|SEC14L2 \|CAPN10 \|CES2 \|APOOL \|BS3ST3A1 | 3.822137246 | 0.24866 | 0.99382 | 0.09468 | 0.08816 | 0.92608 |
| MMAA \|IER5 \|TBPL1 \|SULT1B1 \|CCDC112 \|MAPKBP1 \|RAVER1 \|ZNF765 \|SSX2IP \|MPG | 3.81545969 | 0.28426 | 0.77303 | 0.20136 | 0.52186 | 0.93595 |
| GPR18 \|USP21 \|ARFIP2 \|SAAL1 \|ENOX2 \|MYNN \|GPN2 \|MRPL30 \|LCLAT1 \|TRIM23 | 3.811128061 | 0.26967 | 0.89861 | 0.19112 | −0.2602 | 0.93785 |
| MOCS2 \|BEMGN \|ZNF155 \|DCAF4 \|POLR1A \|IMPA2 \|RING1 \|MBTPS2 \|RCN2 \|VMA21 | 3.80625418 | 0.26539 | 0.72477 | 0.25832 | −0.6618 | 0.94233 |
| TNFAIP1 \|PEX7 \|ZBTB10 \|NSUN3 \|HSPE1-MOB4 \|PRDX2 \|SLC25A15 \|HSPA14 \|BCL11A \|FBXL4 | 3.802049337 | 0.28065 | 0.68008 | 0.23032 | 0.17194 | 0.93317 |
| MAP2K7 \|RECQL5 \|DNAJC5 \|LRP10 \|CDKN2AIP \|WDR19 \|ELK3 \|C7orf50 \|ZNF615 \|MMD | 3.79787469 | 0.2987 | 0.50241 | 0.22033 | −0.9255 | 0.924 |
| TMEM9B \|CACNA2D4 \|RPUSD3 \|TRIM26 \|SMURF1 \|SDE2 \|LRRC8B \|PIK3C2B \|NUP35 \|C7orf26 | 3.793071672 | 0.26818 | 0.90689 | 0.13637 | −0.4697 | 0.9275 |
| PYCR2 \|AKAP11 \|TMEM138 \|TFE3 \|FAM104B \|ZNF678 \|SYNE3 \|FBXO44 \|CSTF2T \|C12orf4 | 3.785597436 | 0.27423 | 0.77582 | 0.1784 | 0.70817 | 0.93047 |
| RSAD1 \|PRPS2 \|ARHGAP10 \|TUBD1 \|SHARPIN \|FBXO32 \|MRPL28 \|ZNF283 \|RNF144A \|NME7 | 3.780728313 | 0.26981 | 0.73995 | 0.22411 | −0.4332 | 0.91963 |
| PSMB5 \|FUT11 \|SIAH2 \|NOC3L \|CCDC15 \|TNFSF8 \|GEMIN2 \|SNED1 \|CLN8 \|FBXL3 | 3.773042744 | 0.27073 | 0.78958 | 0.12909 | −0.5231 | 0.93614 |
| PPP2CB \|CD101 \|URGCP \|SPIN1 \|BLCAP \|USE1 \|XPO4 \|RAB11B \|LMNB2 \|FADS2 | 3.76335026 | 0.2866 | 0.89078 | 0.15162 | −0.5241 | 0.92466 |
| SCARB1 \|ZNF12 \|BRD3 \|PMF1-BGLAP \|CCDC22 \|TRAPPC2L \|DZIP3 \|LRRC8C \|AHI1 \|ZNF839 | 3.760340949 | 0.29316 | 0.74012 | 0.17172 | −0.6483 | 0.92508 |
| SS18L1 \|GNGT2 \|ZNF154 \|ATG2A \|ARHGEF40 \|C12orf10 \|SLC29A1 \|NUP133 \|EIF4EBP1 \|TRAF2 | 3.754999518 | 0.27034 | 0.79621 | 0.11438 | 1.54041 | 0.91689 |
| ERI3 \|BAVCR1 \|TTC16 \|PLEKHA3 \|NUDT1 \|TAB1 \|RNF8 \|PRSS1 \|KIAA1841 \|DNAJC24 | 3.748668688 | 0.26161 | 0.85867 | 0.19298 | 0.11567 | 0.9279 |
| TAF9 \|CCDC65 \|ZNF79 \|PIGV \|ZNF439 \|RNF113A \|NFE2 \|KIF21A \|UNKL \|ZNE358 | 3.743651052 | 0.24916 | 0.96814 | 0.07312 | −0.7809 | 0.92354 |
| MYO6 \|MXD4 \|TANC2 \|GALE \|MRPL2 \|PRDM15 \|EHMT2 \|ZNF585A \|NCLN \|NPEPL1 | 3.737261833 | 0.2671 | 0.95143 | 0.04652 | −1.0152 | 0.92534 |
| CDKL1 \|ZNF438 \|ZNF225 \|DCAF10 \|FAM98B \|NFIA \|PUS1 \|DDX59 \|GSTZ1 \|P2RY13 | 3.731621901 | 0.27394 | 0.88067 | 0.16464 | 0.06935 | 0.93991 |
| CLIC4 \|METTL2A \|REPS2 \|TPRG1 \|KLHL3 \|ISYNA1 \|HSCB \|ATAD2 \|ALDH6A1 \|PPP1R15B | 3.727571649 | 0.26245 | 0.92421 | 0.10074 | 0.60864 | 0.93104 |
| CCDC125 \|TMEM192 \|CR2 \|CD93 \|DHX35 \|NEK1 \|TAMM41 \|SMU1 \|CENPK \|ARMC6 | 3.721626508 | 0.27247 | 0.7061 | 0.24496 | −0.548 | 0.94012 |
| CCDC39 \|COCH \|DMPK \|SQLE \|GPAA1 \|PFDN1 \|CD151 \|BIST1H2AB \|UMPS \|ATP5J2-PTCD1 | 3.716929811 | 0.24473 | 0.99542 | 0.10123 | −0.8207 | 0.92236 |
| HIST4H4 \|TOMM40L \|ZNF419 \|CCDC94 \|APH1B \|TRIM27 \|CA6 \|ADIPOR2 \|LMO2 \|ZNF234 | 3.713212439 | 0.26971 | 0.86978 | 0.10464 | −0.5881 | 0.92904 |
| HRAS \|PTP4A3 \|ACTR3B \|HNRNPA1L2 \|HCFC1 \|ZNF335 \|CHRNB1 \|ZNF511 \|KIAA1586 \|S100B | 3.708380113 | 0.274 | 0.9467 | 0.0781 | −0.9391 | 0.9171 |
| WRAP73 \|STRADB \|PIAS3 \|PHGDH \|SRRD \|ETV3 \|MRPL27 \|PTGER4 \|FADS3 \|FMN1 | 3.703386996 | 0.27103 | 0.81546 | 0.19888 | −0.1462 | 0.92669 |
| NIP7 \|METRNL \|CDK2AP2 \|BDH2 \|ZNF441 \|CAAP1 \|RRP1B \|TMCO4 \|HENMT1 \|ZNF273 | 3.696912921 | 0.27567 | 0.8941 | 0.12774 | −0.2523 | 0.92754 |
| PMEL \|ST3GAL2 \|ATAT1 \|WDR13 \|KRT73 \|SLC25A25 \|PRR5 \|ARID3A \|C3orf58 \|TSR1 | 3.690002445 | 0.24982 | 1.07567 | 0.07725 | −0.7212 | 0.90754 |
| UGGT2 \|ALAS1 \|LIN37 \|FHOD1 \|MRPL16 \|JAKMIP1 \|RHOBTB3 \|PAICS \|RNASEH1 \|CBX1 | 3.681263886 | 0.28965 | 0.97179 | 0.15224 | −0.6883 | 0.9231 |
| GZF1 \|NAA38 \|PAK1IP1 \|ARL4C \|UBAC1 \|IFT172 \|CCDC146 \|GPKOW \|EMC10 \|GATAD1 | 3.676231289 | 0.28184 | 0.59125 | 0.17989 | −0.5709 | 0.93115 |
| VGLL4 \|MRPL53 \|XRCC4 \|ANKAR \|NTS12 \|PDCL \|EPHB6 \|ARL2 \|ELOVL7 \|MCM9 | 3.673148374 | 0.28224 | 1.02537 | 0.10972 | −0.4292 | 0.93436 |
| PDZD11 \|CTBS \|PSMD14 \|KIF16B \|SLC9B2 \|GRAMD1B \|SLC26A11 \|NUS1 \|MST1 \|MEST | 3.667025931 | 0.24743 | 0.94852 | 0.12444 | 0.33023 | 0.92711 |
| COX18 \|ZNF776 \|IGFBP7 \|VAT1 \|SEC22C \|FAM117B \|BICD2 \|DEFA1 \|ZNF398 \|CSRNP1 | 3.660078573 | 0.29214 | 0.76234 | 0.18957 | 1.72879 | 0.92559 |
| TSPAN17 \|BLZF1 \|MRS2 \|PFKFB2 \|FRS2 \|RAB34 \|PGAP2 \|NOP16 \|TSTA3 \|ZNF350 | 3.655105206 | 0.28991 | 0.85509 | 0.1692\| | −0.502 | 0.92521 |
| C7orf43 \|LGALSL \|FUCA2 \|EAF1 \|RAB32 \|ZZZ3 \|SAP30 \|WDR76 \|EZH2 \|RACGAP1 | 3.651216314 | 0.28452 | 0.87101 | 0.16385 | −0.7746 | 0.93015 |
| CACNB1 \|ITM2C \|CCDC97 \|YIPF6 \|FAM58A \|ICOS \|MAP1S \|AKAP7 \|SYNGR1 \|SUPT3H | 3.645771972 | 0.26284 | 0.89231 | 0.12853 | 0.57107 | 0.92125 |
| ZNF594 \|GPANK1 \|MVD \|PQLC1 \|PFAS \|NEBL \|PPRC1 \|SLC35B4 \|PPP2R5B \|INSR | 3.640333839 | 0.26265 | 0.94826 | 0.11123 | −0.2645 | 0.9283 |
| EEF1E1 \|MYO1D \|ABCD1 \|PIGP \|PRSS23 \|GPATCH1 \|ZNF688 \|EMC8 \|SMCR8 \|IFT20 | 3.631481534 | 0.27391 | 0.87977 | 0.15542 | −0.4886 | 0.92923 |
| TMEM68 \|RPP38 \|TRIM13 \|GFI1B \|BTBD2 \|NABP2 \|TFAP4 \|CLEC10A \|RHOBTB2 \|TNNT1 | 3.62421474 | 0.24006 | 0.98247 | 0.08277 | −0.0817 | 0.9155L |
| ARSA \|ACY1 \|ING2 \|HFE \|NIPA2 \|COL9A2 \|C19orf33 \|CHERP \|TMTC3 \|HMGCS1 | 3.618458327 | 0.27852 | 0.74267 | 0.21196 | 0.07922 | 0.91929 |
| SLC9A1 \|MED22 \|CD1D \|FXYD6 \|MAN1C1 \|MAST2 \|CD300C \|KCTD7 \|DVL2 \|MRPL44 | 3.611553904 | 0.2582 | 0.92128 | 0.05825 | 0.94909 | 0.91965 |
| MTMR7 \|TMEM106B \|SLC26A6 \|GOLM1 \|HSDL2 \|CHI3L2 \|TCEAL3 \|CEP70 \|C7orf49 \|COQ10B | 3.606206739 | 0.25271 | 0.7377 | 0.12467 | −0.4145 | 0.93119 |
| KLHDC1 \|PHACTR1 \|ARID5A \|PDE4A \|HOOK2 \|NLRC4 \|CREM \|OSBPL7 \|TOE1 \|TNIP2 | 3.601688582 | 0.25724 | 0.96036 | 0.073 | 0.12314 | 0.92795 |
| ARHGAP32 \|C9orf3 \|CASP3 \|LRP5L \|PNO1 \|THBS3 \|SLC37A1 \|PTPMT1 \|PPP1R138 \|ZNE577 | 3.596113646 | 0.23131 | 0.95221 | 0.08902 | −0.1412 | 0.92982 |
| FOXO4 \|B3GAT3 \|POP7 \|LIAS \|RNF157 \|TMEM241 \|PLAGL1 \|RASSF1 \|C7orf73 \|F2R | 3.591663228 | 0.27621 | 0.76374 | 0.16945 | −0.9997 | 0.93082 |
| ZMAT1 \|ZNF268 \|MITF \|MTMR9 \|TMEM38B \|TIRAP \|BTBD11 \|RASGRP3 \|SLC44A1 \|NEB | 3.586519526 | 0.26013 | 0.93788 | 0.06594 | 0.71617 | 0.94274 |
| MIEN1 \|IRF7 \|SOCS7 \|ZNF568 \|BOD1 \|USP19 \|SAMD1 \|CHURC1-FNTB \|ORA12 \|AXIN2 | 3.581591104 | 0.28136 | 0.77426 | 0.13116 | −0.6942 | 0.92185 |
| NPRL3 \|ACSM3 \|TAS2R43 \|HERC6 \|FBXO8 \|SLC25A14 \|AKIRIN2 \|LSM12 \|SGMS1 \|ZNF490 | 3.577188668 | 0.28351 | 1.10547 | 0.04286 | −0.4648 | 0.94208 |
| EIF1AX \|ZNF486 \|NEIL1 \|TMEM18 \|CIDEB \|DARS2 \|TRIM73 \|NSDBL \|RNASEL \|FGFBP2 | 3.570040795 | 0.27289 | 1.07562 | 0.10007 | −1.0165 | 0.9264 |
| RBM42 \|CEP85 \|ECI1 \|ZFAND2A \|CTIF \|GALNT21 \|ITGB3BP \|SIRT3 \|KRTAP5-1 \|CYP27A1 | 3.564217048 | 0.27198 | 0.75276 | 0.17276 | −0.8918 | 0.92753 |
| CLCN5 \|RDH14 \|ARID3B \|UTP15 \|PDK3 \|SLC41A3 \|YIPF4 \|ALG10B \|FASTK \|RLF | 3.556692021 | 0.27284 | 0.69398 | 0.22114 | 0.27078 | 0.93001 |
| ASTE1 \|MRPL38 \|UEVLD \|RPP14 \|CENPJ \|RGP1 \|NDUFB11 \|ZNF691 \|IFIT2 \|C2orf42 | 3.553144552 | 0.26962 | 0.91549 | 0.13125 | −0.6453 | 0.93914 |
| HERC5 \|FN3KRP \|TMEM55B \|KIF20B \|DBN1 \|CHKA \|PYGM \|TFEB \|PGA5 \|ZNF639 | 3.546406747 | 0.27433 | 0.77573 | 0.08592 | −0.6643 | 0.91882 |
| F8A1 \|OSBPL10 \|RANBP10 \|TECPR2 \|RBBP5 \|C8orf33 \|ZNF146 \|L3MBTL1 \|GAGE2A \|DBR1 | 3.538088018 | 0.26426 | 0.76064 | 0.20946 | −1.0116 | 0.92816 |
| ZNF140 \|TRIQK \|KIN \|CUBN \|RALGPS1 \|DAG1 \|RFPL4A \|HEATR5A \|FAM129A \|MTMR6 | 3.528695279 | 0.2583 | 0.86441 | 0.06725 | 0.91484 | 0.94428 |
| TAF12 \|IFT74 \|RAP2A \|NGRN \|ARRDC2 \|PTPN23 \|RFC5 \|OAZ2 \|NET1 \|WNK4 | 3.524548457 | 0.28077 | 0.7186 | 0.17419 | 0.25264 | 0.92862 |
| AKR1E2 \|ZMPSTE24 \|ZYG11B \|ERF \|CEP41 \|POFUT1 \|ADCY9 \|HDAC5 \|ANKS1A \|FCRL2 | 3.52004834 | 0.27304 | 0.75374 | 0.17391 | −0.747 | 0.92411 |
| ANAPC13 \|CSTF2 \|ZNF687 \|EMC9 \|ORC5 \|ATG4D \|DEFA \|MIDN \|TTC33 \|PLA2G16 | 3.514088911 | 0.29416 | 0.81761 | 0.25135 | −0.7533 | 0.93351 |
| MPPE1 \|C8orf44-SGK3 \|ANAPC2 \|YWHAG \|OVCA2 \|RAB22A \|LRRC25 \|EED \|GET4 \|TRIM35 | 3.507206996 | 0.2953 | 0.73437 | 0.16069 | −0.2891 | 0.91861 |
| RBM12B-AS1 \|PARP11 \|ICK \|CNPY4 \|ZNF709 \|GLO1 \|ATXN1L \|PER2 \|PPT2 \|NARFL | 3.499370308 | 0.28664 | 0.65705 | 0.16893 | −0.4418 | 0.93026 |
| TTC21A \|SNTB2 \|CLYBL \|KPNA3 \|THAP7 \|JUP \|PPP1R32 \|CHRNG \|STK19 \|HIST1H3B | 3.493442726 | 0.26348 | 0.82153 | 0.12777 | −0.1672 | 0.91894 |
| GCFC2 \|TRAF4 \|BOLA3 \|CRK \|ZNF136 \|MAPK7 \|YARS2 \|PLBD2 \|TULP4 \|INPP1 | 3.488681232 | 0.26735 | 0.92553 | 0.17332 | −0.7726 | 0.92883 |
| EEFSEC \|ZNF529 \|SLC25A44 \|PDE9A \|PGRMC1 \|DOCK4 \|NCEH1 \|MRPL46 \|TAF4 \|DNAJC14 | 3.483742577 | 0.27535 | 0.8023 | 0.18423 | −0.0354 | 0.92388 |
| LIG4 \|CCDC170 \|PAXIP1 \|WDR49 \|GOLPE3L \|SNAPIN \|RNF126 \|FAM20A \|NUP37 \|IFT46 | 3.476541966 | 0.25276 | 0.92821 | 0.04777 | −0.4892 | 0.93794 |
| PRIM2 \|PLEKHG4 \|GGPS1 \|AIMP2 \|IQCE \|MAL \|SLC35E2B \|CRTAM \|CCNYL1 \|NUSAP1 | 3.470511106 | 0.2571 | 0.95958 | 0.10473 | 1.74053 | 0.92777 |
| CDK5R1 \|GNRH1 \|GCOM1 \|KCNC3 \|KRT72 \|PJA1 \|CPNE2 \|CEACAM3 \|ZNF507 \|LSR | 3.46594451 | 0.22852 | 1.01459 | 0.0886 | −0.2469 | 0.9252 |
| C12orf66 \|ZNE461 \|ANKRD40 \|TBRG4 \|GOLGA3 \|ASGR1 \|CDYL2 \|RHOH \|MRPL34 \|IFIT1 | 3.459214475 | 0.25959 | 0.86495 | 0.16808 | −0.37 | 0.93147 |
| WRB \|MPND \|MANEA \|RGL4 \|ZNF566 \|DGKG \|TOP3B \|HORMAD1 \|CAMLG \|CREB3 | 3.451569807 | 0.2605 | 0.92813 | 0.0954 | −0.3747 | 0.93008 |
| NMT2 \|RANGAP1 \|BICD1 \|DBRS3 \|SMPDL3A \|HLA-DQA2 \|MZT2A \|ZNF844 \|UQCRES1 \|MTRF1L | 3.447320767 | 0.26677 | 0.91957 | 0.12451 | −0.9008 | 0.92822 |
| TAS2R31 \|AGPAT1 \|MYL9 \|MRPS28 \|ZNF484 \|MOB3C \|TCEA3 \|ZNF672 \|TMEM40 \|C22orf39 | 3.440466156 | 0.25034 | 1.07726 | 0.0742 | 0.44683 | 0.91867 |
| AARS2 \|FBXO31 \|RMNDSB \|SLC12A2 \|GNPDA1 \|LSM2 \|ABRACL \|NIPA1 \|FLYWCH1 \|DDAH2 | 3.436242471 | 0.29153 | 0.71457 | 0.12287 | −0.2519 | 0.92476 |
| FLYWCH2 \|RNF217 \|RCL1 \|ZNF3 \|FASTKD1 \|SRC \|TATDN2 \|THAP4 \|ZNF526 \|SPATS2 | 3.430448338 | 0.2605 | 0.82317 | 0.07391 | −0.5778 | 0.92996 |
| LRRC27 \|FAM98C \|TDRD9 \|VSTM1 \|RNF144B \|DNAJB4 \|KRT5 \|ENKUR \|KDM5B \|DHX33 | 3.422992496 | 0.24572 | 1.08107 | 0.13094 | −0.7168 | 0.93776 |
| CD160 \|TICAM1 \|SRFBP1 \|C19orf47 \|GARNL3 \|DTWD1 \|PIGA \|FUOM \|PCK2 \|MRPS2 | 3.416624975 | 0.25581 | 0.87527 | 0.09178 | 0.11154 | 0.93013 |
| SPATA5L1 \|ZNF557 \|NHS \|ZNE256 \|WBP4 \|TARBP2 \|ZSCAN16 \|NCALD \|ACTR3C \|RNASE4 | 3.412034293 | 0.27435 | 0.98337 | 0.1191 | −1.0101 | 0.92922 |
| C11orf1 \|C22orf34 \|RNF165 \|PIGF \|RABEPK \|CKS1B \|TFPT \|PHTF1 \|FARSB \|DHFR | 3.407038741 | 0.28456 | 0.82272 | 0.11104 | 0.2028 | 0.92663 |
| TRUB2 \|WDYBV1 \|USP38 \|TPST2 \|BARD1 \|SS18L2 \|DCUN1D5 \|CCDC107 \|NEK8 \|FAM3A | 3.401211087 | 0.30078 | 0.68384 | 0.27511 | −0.7618 | 0.93769 |
| CXXC5 \|WDR53 \|ACOT2 \|KIAA2013 \|TADA1 \|FHL1 \|HEATR6 \|SNAPC4 \|TELO2 \|NME6 | 3.394123542 | 0.26905 | 0.8616 | 0.22965 | −0.7069 | 0.92535 |
| ZNF823 \|C21orf59 \|BHLHE41 \|C17off80 \|SETD1A \|TMTC2 \|ANO9 \|SEMA3C \|ZNF770 \|ZNF320 | 3.388797391 | 0.26195 | 0.9688 | 0.03499 | −0.5613 | 0.91872 |
| NEO1 \|ABCG1 \|DDIT4 \|PAQR3 \|PCBD \|UTP23 \|IQSEC2 \|POLA1 \|GPN3 \|CEP57L1 | 3.384019517 | 0.27419 | 0.84888 | 0.15213 | −0.5519 | 0.92569 |
| ZFPM1 \|NTPCR \|MUC12 \|THAP11 \|COX16 \|SNX24 \|STRC \|INSIG2 \|BTD \|ZNF25 | 3.378368091 | 0.25016 | 0.76674 | 0.10208 | 0.2127 | 0.93287 |
| ZNF282 \|JMJD7-PLA2G4B \|INTS2 \|USP53 \|BLA-DOA \|PDK4 \|ZNF280C \|NRG1 \|EFHC1 \|C2orf49 | 3.370972407 | 0.26591 | 0.84693 | 0.18338 | −0.0245 | 0.93696 |
| MRPL15 \|AKAP2 \|NR2C2AP \|ALK \|ALKBH8 \|PHYH \|KLC4 \|GBE1 \|SLC3A1 \|POLD4 | 3.366885131 | 0.24499 | 1.00917 | 0.09967 | 1.18142 | 0.93029 |
| CHRAC1 \|NFATC1 \|CCP110 \|DCAKD \|NOC4L \|SLC20A2 \|ACP6 \|RBFA \|CGRRF1 \|URM1 | 3.360897772 | 0.27772 | 0.68298 | 0.24408 | 0.38313 | 0.91987 |
| PWP2 \|SEMA6A \|FAM122C \|CUEDC1 \|LLGL2 \|HLCS \|SYAP1 \|ZBTB20 \|ZNF248 \|STMN3 | 3.355203906 | 0.26781 | 0.58815 | 0.23541 | −0.6141 | 0.92503 |
| ANKRD46 \|ZNF17 \|CMTM1 \|EPM2A \|PGPEP1 \|TAF5L \|ABL2 \|CCDC142 \|FANCM \|ANKRD42 | 3.347820374 | 0.2664 | 0.91295 | 0.07475 | −0.6547 | 0.93512 |
| SPATA7 \|BDH1 \|SRRM5 \|POLL \|B9D2 \|GPATCH2 \|CERS4 \|NARS2 \|ERP27 \|BCL2L12 | 3.341579501 | 0.2715 | 0.94872 | 0.1343 | 0.07195 | 0.92445 |
| C1D \|FRMD8 \|ZNF395 \|CYP3A5 \|PCTP \|ACYP1 \|FGD6 \|YAE1D1 \|EMC6 \|ENOPH1 | 3.333124995 | 0.28209 | 0.81169 | 0.18983 | −1.0561 | 0.93745 |
| E2F5 \|CCNB1IP1 \|UBE2O \|QSOX2 \|PPME1 \|TMEM41B \|CNNM2 \|FCER2 \|FBXO4 \|FAM86B1 | 3.32329731 | 0.27825 | 0.78179 | 0.15856 | −0.3395 | 0.93733 |
| ATP6V0E2 \|C16off54 \|POLR3F \|ZNF649 \|KCTD3 \|NAALADL1 \|ZFYVE1 \|ASAH2 \|SLC39A3 \|DNAJC9 | 3.31563891 | 0.23677 | 1.01471 | 0.12856 | −0.7878 | 0.93169 |
| MARCO \|CASP5 \|IL27RA \|IMMP1L \|CROCC \|NSUN4 \|DHX34 \|SNX9 \|ZNF573 \|USP12 | 3.3089702 | 0.23016 | 1.02068 | 0.08825 | −0.60$7 | 0.9293 |
| LYRM1 \|MFAP3L \|SNIP1 \|ZC3HC1 \|LIMA1 \|DNAJC15 \|HSD11B2 \|AP1AR \|OSCP1 \|LIN7A | 3.303731971 | 0.28348 | 0.84038 | 0.15943 | −0.2723 | 0.93319 |
| SRF \|NPAS2 \|NOL9 \|SYCP2 \|CASP7 \|RAB15 \|TOR3A \|ATP7A \|RELB \|TTC8 | 3.296079056 | 0.27646 | 0.74503 | 0.16844 | −0.6578 | 0.91557 |
| C4orf33 \|JOSD2 \|MAK \|CRYL1 \|TMEM222 \|JMJD7 \|CLIC2 \|ULK1 \|SWT1 \|SLC35A2 | 3.291357608 | 0.29506 | 0.76797 | 0.14271 | 0.38884 | 0.93231 |
| MPZL3 \|LSM1 \|POLR2D \|TGDS \|KLHL9 \|BCAT1 \|SLC9A7 \|HMGN5 \|TRIM38 \|CACTIN | 3.285958819 | 0.28096 | 0.78206 | 0.18958 | −1.0365 | 0.93879 |
| DRAM1 \|CRIM1 \|HOMER3 \|APOBR \|BCL7A \|TBL3 \|PPTC7 \|PVRIG \|RFK \|POLR1E | 3.278513228 | 0.25041 | 0.91625 | 0.08208 | −0.3774 | 0.91327 |
| SLC39A4 \|CYP17A1 \|MED11 \|METTL15 \|BAK1 \|RPA3 \|FNBP1L \|PEX13 \|FMO5 \|HLTF | 3.271646653 | 0.24853 | 1.03589 | 0.12095 | 0.87566 | 0.92657 |
| ZNF433 \|TBC1D10A \|CP \|XYLT2 \|STAP1 \|PLEKHA5 \|CEP44 \|TEC \|DNASE1L1 \|C1QA | 3.263924359 | 0.24445 | 1.0245 | 0.11621 | −0.1226 | 0.93161 |
| MYB \|MRTO4 \|ZFHX3 \|PDCD7 \|FABP5 \|F2RL2 \|RNF103 \|MTHFD2L \|VDR \|BTG3 | 3.255557163 | 0.26473 | 0.86825 | 0.10899 | 0.16177 | 0.92891 |
| TAOK2 \|ZNF331 \|ZUFSP \|PHLDB3 \|DAPK3 \|FGFR1 \|RENBP \|VAV2 \|MRC1 \|ZNF510 | 3.24669766 | 0.26883 | 0.81336 | 0.13455 | 0.29494 | 0.91013 |
| TOLLIP \|PBXO25 \|ACVR18 \|ATP5S \|N48P2 \|TEFM \|LRRC28 \|PRKAR1B \|ADAM22 \|OCRL | 3.241632736 | 0.26936 | 0.81772 | 0.1172 | −0.8053 | 0.92077 |
| CCR1 \|ZFP57 \|KATNAL1 \|NUDT16 \|IFRD2 \|STXBP1 \|LETM2 \|RNF187 \|ZBTB2 \|ZNF598 | 3.235707337 | 0.28456 | 0.63761 | 0.20966 | 0.01472 | 0.92725 |
| STARD10 \|PGAP3 \|KLHL6 \|TTI1 \|ABCB8 \|IMPAD1 \|MRAS \|PEAK1 \|C9orf47 \|PELP1 | 3.228611167 | 0.27242 | 0.82056 | 0.13883 | −0.925 | 0.93213 |
| SLFN12 \|FBXW8 \|SACS \|SCLY \|ZNF75D \|ZBTB41 \|SNX20 \|HBEGE \|AIG1 \|NFS1 | 3.222059684 | 0.262 | 0.97323 | 0.12228 | −0.5699 | 0.93396 |
| LTBP1 \|GALNT12 \|VEGFB \|FAIM \|MRPL13 \|RHOU \|ZNF516 \|UNC45A \|ZNF23 \|SYNJ2 | 3.212663575 | 0.26042 | 0.95438 | 0.09568 | −0.1434 | 0.92108 |
| FAM102B \|OPN3 \|TBC1D7 \|ITGAE \|ZNF496 \|FKBP9 \|RBM48 \|ALDH7A1 \|RRP15 \|ACE | 3.2089702 | 0.24627 | 0.9263 | 0.12698 | 1.58798 | 0.93071 |
| SURF6 \|ASNS \|CTNNBIP1 \|BEX2 \|STARD4 \|C3 \|CYP51A1 \|THOP1 \|MAPK13 \|FAF2 | 3.204179401 | 0.27856 | 0.92704 | 0.0941 | −0.8143 | 0.92767 |
| RBAK \|COQ7 \|PARP3 \|CARHSP1 \|ZDHHC14 \|UTP20 \|GPR65 \|ZMAT5 \|MMS22L \|ZNF324 | 3.19928552 | 0.28624 | 0.80418 | 0.14412 | 2.23404 | 0.9257 |
| RPL36A-HNRNPB2 \|SH2D1B \|CTSO \|TADA2B \|COIL \|MECR \|UQCR11 \|DCK \|TCTN2 \|SLC4A8 | 3.191746291 | 0.26145 | 0.94483 | 0.09363 | −0.7936 | 0.93648 |
| TMEM63C \|CD320 \|ITGB5 \|TBC1D20 \|TAF13 \|QRSL1 \|TEX10 \|FBXO6 \|BAG3 \|GPR52 | 3.185015232 | 0.24783 | 0.86588 | 0.15488 | 0.71571 | 0.93133 |
| C19orf70 \|MFGE8 \|ZDHHC11 \|TUBB2A \|THG1L \|APBA3 \|PGA3 \|ELL \|ZADH2 \|TTC7B | 3.180038161 | 0.25646 | 0.95065 | 0.08431 | −0.239 | 0.9174 |
| OLFM1 \|ZNE426 \|RNF169 \|FABP3 \|CEACAM4 \|TP5I3 \|WRAP53 \|MED16 \|C3AR1 \|RNF219 | 3.174612004 | 0.23244 | 0.88937 | 0.13233 | −0.1961 | 0.91994 |
| ASNSD1 \|CFH \|ABCB10 \|HPGD \|SLC18B1 \|PDE12 \|ADAMTS1 \|BMF \|MLANA \|SPIB | 3.169054195 | 0.24238 | 1.04235 | 0.0302 | 0.55234 | 0.93054 |
| PLEKHM3 \|USP13 \|ADCK1 \|CCDC149 \|MT1E \|CNTLN \|FAR2 \|NHLRC2 \|KRTCAP3 \|GPRIN3 | 3.163248656 | 0.24466 | 0.96455 | 0.0874 | 0.63648 | 0.92858 |
| SLC9B1 \|TOMM40 \|DUSP23 \|ACP2 \|ZNF790 \|WDR73 \|RCBTB1 \|METTL6 \|CNKSR2 \|SLC3543 | 3.156637144 | 0.27369 | 0.87626 | 0.18274 | 0.34066 | 0.93146 |
| RAD51 \|TMEM97 \|TSEN15 \|CYB5D2 \|CASP9 \|PRG4 \|WDR47 \|LRRC59 \|METTL21A \|TMEM9 | 3.151296422 | 0.28151 | 0.79264 | 0.1597 | −0.7207 | 0.93684 |
| REEP4 \|CISD3 \|TRIM68 \|HS1BP3 \|PVALB \|SNX18 \|GAGE2E \|UBQLN4 \|RPSAP58 \|CDK5 | 3.147608435 | 0.27116 | 0.82272 | 0.14429 | −0.2435 | 0.9169 |
| SLC6A16 \|KIAA1683 \|PHF2 \|TOP1MT \|SEPHS1 \|UBL3 \|GIGYF1 \|SLC35E3 \|DNAJB12 \|CRELD2 | 3.143258789 | 0.25454 | 0.89096 | 0.14277 | 0.69322 | 0.90717 |
| DPH2 \|DHX37 \|ABCC6 \|ZNF347 \|PTRHD1 \|PKIG \|NICN1 \|PPP1R14B \|MRPS10 \|ASB7 | 3.136267465 | 0.27877 | 0.76482 | 0.23558 | −0.7297 | 0.92881 |
| STK16 \|HELLS \|FANCC \|PAPSS2 \|PHLDB2 \|TCN2 \|TMEM170A \|PTPRCAP \|AP4S1 \|ZNF286B | 3.127868487 | 0.25197 | 0.95864 | 0.00917 | −0.272 | 0.9252 |
| CRAT \|HYLS1 \|CCDC92 \|YIPF2 \|AP5Z1 \|NDUFA7 \|MUT \|OSGEPL1 \|TOX \|TMEM234 | 3.121604292 | 0.28776 | 0.66197 | 0.2363 | −0.6934 | 0.92104 |
| ANKMY2 \|CXCL16 \|POLD1 \|NENE \|C9orf85 \|HAUS6 \|PCYOX1L \|MKKS \|BORA \|CMC2 | 3.113672745 | 0.29597 | 0.65307 | 0.16013 | 0.09966 | 0.9237 |
| HIST1H41 \|ACO1 \|MDP1 \|GOT2 \|GTF2H4 \|ALS2CL \|CETP \|FRMD3 \|ZNF175 \|MME | 3.109042256 | 0.25105 | 0.82253 | 0.22577 | 0.16714 | 0.92371 |
| ZKSCAN5 \|MTX3 \|TRIM65 \|PSMD10 \|MGAT5 \|SRM \|FAM129B \|DTYMK \|CARD6 \|YPEL1 | 3.099614278 | 0.28305 | 0.72799 | 0.16945 | 1.16607 | 0.92889 |
| LYPD2 \|TPRA1 \|TMUB2 \|TIGD1 \|ELL2 \|FAM72A \|ZNF142 \|MBD3 \|ZEYVE21 \|NEK3 | 3.092768408 | 0.27471 | 0.87466 | −0.0248 | −0.8509 | 0.91522 |
| PCBD2 \|NREP \|NFXL1 \|WDR4 \|LRWD1 \|LIN7C \|DESI2 \|HABP4 \|STK3 \|BTBD3 | 3.085980735 | 0.26308 | 0.86321 | 0.15102 | −0.117 | 0.93763 |
| PTDSS2 \|NLRP2 \|MPV17L2 \|POGK \|PLCB3 \|HSD17B8 \|SRD5A3 \|ZNF623 \|ZMAT3 \|KDSR | 3.076992976 | 0.26972 | 0.99321 | 0.04906 | −0.9134 | 0.9221 |
| TAF3 \|ARMC10 \|CXorf57 \|DCTPP1 \|SSH3 \|ANKRD6 \|EVI5L \|NRIP2 \|ZBTB7A \|HIST1H3J | 3.069051642 | 0.24036 | 0.88746 | 0.14201 | −0.1289 | 0.9215 |
| ZHX1 \|FAM206A \|VPS33A \|DCBLD1 \|RNF115 \|VPREB3 \|PAM16 \|SLC4A5 \|UHRF1BP1 \|SCYL3 | 3.062764614 | 0.26213 | 0.84361 | 0.11219 | −0.4882 | 0.92999 |
| TMEM189-UBE2V1 \|ZNF653 \|CDKN2D \|IGSF9B \|NAPEPLD \|TRMT44 \|ZNF445 \|EOGT \|NAF1 \|CXCR2 | 3.055523612 | 0.26692 | 0.86849 | 0.08206 | −0.7197 | 0.92924 |
| PCYT2 \|HNRNPUL2 \|KIAA1147 \|EP400NL \|HAUS8 \|ZNF626 \|BAIAP2 \|ZNF134 \|ARHGEF35 \|SH2B2 | 3.047113789 | 0.27956 | 0.80046 | 0.10112 | −0.6535 | 0.91013 |
| TSPAN4 \|POLR3G \|PRDM10 \|LAMC1 \|PDE6D \|MPZL1 \|ABL1 \|CMC1 \|GDPD1 \|ST5 | 3.042911557 | 0.27389 | 0.8537 | 0.16301 | −0.9404 | 0.92081 |
| LRRK1 \|TMEM208 \|IGFN1 \|DLEU1 \|LATS2 \|CCDC61 \|LRRC40 \|MRPL10 \|CD83 \|SLC9A8 | 3.035610676 | 0.25693 | 0.7935 | 0.15784 | 0.19996 | 0.92918 |
| FCRLA \|STAMBPL1 \|STON2 \|HSBP1L1 \|MGMT \|SCAMP4 \|IFTT5 \|ZNF773 \|CEP68 \|VNN3 | 3.02969129 | 0.26029 | 0.93109 | 0.12291 | −0.6678 | 0.93295 |
| FASTKD5 \|ANKRD52 \|STAG3 \|AP1S1 \|GIN1 \|PPP1R11 \|ZNF570 \|POU2AF1 \|ZNF674 \|SLC37A4 | 3.023236899 | 0.26873 | 0.8458 | 0.1238 | −0.4748 | 0.92345 |
| L2HGDH \|STX6 \|DOLK \|ZSCAN30 \|DLG4 \|ANG \|PLCL1 \|CRYBG3 \|DPM3 \|MKRN2 | 3.016816015 | 0.26579 | 0.81268 | 0.11668 | 0.13746 | 0.93304 |
| BMPR1A \|SUOX \|VAMP2 \|SLC16A4 \|TMEM150A \|MCM6 \|CHRNA2 \|DSC2 \|SH2D3A \|GPATCH3 | 3.01011935 | 0.2433 | 1.13997 | 0.05344 | 0.28527 | 0.92424 |
| TMEM218 \|FAM173B \|NSUN5 \|DDX51 \|TMEM110 \|CREB3L4 \|C15orf38-AP3S2 \|SLC25A1 \|COX14 \|IL21R | 3.003449885 | 0.27515 | 0.79655 | 0.09666 | 0.12243 | 0.91752 |
| ZSCAN29 \|SLC35B2 \|SLC25A17 \|MAN1A1 \|CLP1 \|SOX13 \|NAA30 \|ASAH2B \|NECAB3 \|PGBD2 | 2.996490158 | 0.27478 | 0.88724 | 0.11221 | −0.4873 | 0.9178 |
| MZB1 \|MRPS36 \|CYTH3 \|GTF2F2 \|LRIF1 \|PHLPP2 \|EIF5A2 \|TGFB3 \|NRAS \|LONRF1 | 2.990106958 | 0.26356 | 0.95858 | 0.16309 | −0.8258 | 0.93289 |
| LRRC6 \|CACNA2D2 \|ZW10 \|RAB40C \|HS2ST1 \|SLC16A5 \|STARD9 \|HEXIM1 \|HSPA6 \|PDGFD | 2.98436213 | 0.25874 | 0.97037 | 0.06029 | −0.5403 | 0.91782 |
| SNX8 \|GADD45GIP1 \|NOP9 \|UNG \|ARPC5L \|ZNHIT6 \|OSCAR \|CNKSR1 \|CEP89 \|ZNF682 | 2.979063241 | 0.2553 | 0.89095 | 0.11188 | −0.4485 | 0.92418 |
| RDH10 \|SLC25A32 \|GNPDA2 \|SPINT1 \|GPRASP1 \|CHPF2 \|MAP3K10 \|BET1 \|MLST8 \|FAM19A1 | 2.972975889 | 0.26741 | 0.94125 | 0.11832 | −0.727 | 0.92679 |
| ZNF860 \|NEDD4 \|ZNF622 \|ZNF540 \|SLC7A5 \|C20orf194 \|ZNF624 \|RPGRIP1L \|RPS19BP1 \|TXNL4B | 2.961544625 | 0.25794 | 0.80045 | 0.15181 | −0.6238 | 0.93604 |
| SLC2A6 \|CDRT1 \|H2AFB3 \|TMEM223 \|WDR92 \|NLGN3 \|TGFBRAP1 \|PPM1L \|RPS6KA2 \|ZNF746 | 2.95468376 | 0.23455 | 0.87008 | 0.08061 | 0.13232 | 0.92816 |
| DGCR6L \|RAD18 \|AIM2 \|CADM1 \|NOL6 \|TBC1D2 \|SPHK2 \|PAFAH1B3 \|ARMC1 \|NMRK1 | 2.947661313 | 0.28554 | 0.68318 | 0.25414 | −0.6837 | 0.93707 |
| HAAO \|NME4 \|ACSS1 \|RYBP \|DOK3 \|TCEAL8 \|ZNF563 \|ZBTB43 \|ANO8 \|FASLG | 2.939444631 | 0.25455 | 0.89665 | 0.16764 | 0.87447 | 0.91154 |
| SIAH1 \|LYSMD2 \|NETO2 \|ZFP14 \|YEATS4 \|GXYLT1 \|ZNF197 \|OPHN1 \|ADAMTS17 \|MED20 | 2.932511973 | 0.26119 | 0.86959 | 0.1659 | 0.02014 | 0.93514 |
| MOSPD3 \|RDH5 \|POLR3H \|LMAN2L \|TULP3 \|IL15 \|GOPC \|SLX4 \|ADAT2 \|TMEM11 | 2.928523864 | 0.28229 | 0.76033 | 0.10201 | −0.8002 | 0.92745 |
| RDH13 \|NCR3 \|BCL9 \|NBEAL1 \|PTCD2 \|RILPL2 \|ZNF212 \|PHLPP1 \|LCN2 \|MALSU1 | 2.923729172 | 0.26407 | 0.63958 | 0.20177 | −0.2837 | 0.91653 |
| ATP10A \|TBX19 \|ZNF619 \|STK32C \|SFXN2 \|MUC6 \|MRPS23 \|PDGFC \|GBGT1 \|ALKBH5 | 2.917369161 | 0.24631 | 0.85343 | 0.10975 | −0.618 | 0.9083 |
| HSPBP1 \|MXRA8 \|PGAM5 \|MED12L \|NFIL3 \|ALDH16A1 \|RLBP1 \|JUN \|AGPAT2 \|APOO | 2.910644027 | 0.25609 | 0.96856 | 0.08619 | −0.7771 | 0.90358 |
| PITPNM2 \|PNPLA4 \|RHCE \|PGRMC2 \|FAHD2B \|BASP1 \|LMO4 \|VASH1 \|ZNF174 \|ANKRD23 | 2.901688248 | 0.2401 | 0.9968 | 0.03507 | −0.8682 | 0.91939 |
| ACOT8 \|USP17L2 \|PLCH2 \|AVEN \|DUSP10 \|DTNA \|ZNF189 \|MBOAT2 \|KIAA1324L \|VANGL1 | 2.894375103 | 0.23106 | 0.84136 | 0.09951 | 2.03197 | 0.92779 |
| DUSP16 \|GDPD3 \|ACAT2 \|ZNF432 \|APIP \|SCRIB \|PTTG2 \|LRRC45 \|MED30 \|SLC10A3 | 2.886995368 | 0.26183 | 0.91105 | 0.13244 | 0.03051 | 0.9289 |
| H1FX \|CHML \|NXPE3 \|SARM1 \|TAF4B \|SLC35B3 \|SMTNL1 \|DCAF16 \|SYNPO \|PRB3 | 2.87878159 | 0.19855 | 1.05156 | 0.02752 | −0.3953 | 0.93544 |
| TMED3 \|CTTNBP2NL \|ACSL6 \|TRIM16 \|CCDC115 \|LEPR \|PSEN2 \|TMTC4 \|STYX \|GTPBP8 | 2.873232427 | 0.27378 | 0.75079 | 0.18194 | 0.43472 | 0.93001 |
| CABP4 \|SLC46AL \|FUNDC1 \|TSPAN5 \|TTPRIPL1 \|NAT1 \|C16orf52 \|RWDD3 \|THEM5 \|RAD1 | 2.862421427 | 0.23264 | 0.92551 | 0.10116 | −0.3664 | 0.918 |
| FAM219B \|KLF16 \|MSANTD3 \|BAIAP3 \|DYNC2LI1 \|MRFAP1L1 \|ZNF879 \|LGMN \|CD9 \|SPRED1 | 2.853690439 | 0.25092 | 1.0798 | 0.0902 | −0.6518 | 0.92416 |
| THAP3 \|FBXO34 \|AKIP1 \|C17orf75 \|FAM222B \|TNFRSF13B \|SASH1 \|ZNF664 \|C1orf112 \|ISOC1 | 2.848486166 | 0.25569 | 0.97781 | 0.02307 | −0.6725 | 0.92709 |
| GRIP1 \|DIEXF \|KCNN4 \|ANKRD50 \|TTPAL \|ZNF418 \|TBCC \|C19orf60 \|FSD1L \|SLC25A19 | 2.840874444 | 0.24864 | 1.01129 | 0.08718 | −0.5691 | 0.92956 |
| LACTB2 \|PDZK1IP1 \|CXCR6 \|GEMIN5 \|CPNE5 \|ZNF420 \|TPPP3 \|NLRX1 \|CCR5 \|TRIP10 | 2.835482456 | 0.24328 | 1.0243 | 0.0774 | −0.7568 | 0.91498 |
| ING1 \|RUNDC1 \|FBN2 \|BAX \|PIGQ \|BRMS1L \|FANCE \|TLR7 \|ZNF18 \|MICALL1 | 2.827492942 | 0.2765 | 0.80093 | 0.12287 | −0.4611 | 0.92244 |
| C2orf76 \|CYHR1 \|GOT1 \|UBL4A \|GALNT6 \|ISCA1 \|MAD2L1BP \|BCL11B \|PEMT \|TG | 2.821578214 | 0.25658 | 1.1163 | 0.0243 | −0.6103 | 0.91852 |
| PIK3R2 \|TXNDC16 \|GCLM \|LSM5 \|RAB4B \|ICA1L \|POP1 \|BCL7C \|FAM69A \|CPEB2 | 2.814313353 | 0.29232 | 0.76964 | 0.17066 | −1.0557 | 0.93128 |
| JMJD4 \|CDC42BPB \|SVIP \|PLAC9 \|FRRS1 \|ZNF184 \|ATF5 \|ZNF699 \|ZNF527 \|FBF1 | 2.804746602 | 0.2651 | 0.86265 | 0.13566 | −0.3394 | 0.93037 |
| CBX7 \|DONSON \|NT5DC3 \|CEP97 \|ATP2A1 \|ABHD11 \|PLEKHB1 \|ST3GAL1 \|NINJ2 \|ZNF223 | 2.798468335 | 0.26247 | 0.88696 | 0.07893 | −0.8483 | 0.91411 |
| LENG1 \|RBM47 \|USP6NL \|SLC22A4 \|ETAA1 \|S100Z \|IL1R2 \|B4GALT5 \|CNTNAP2 \|TIMM13 | 2.790840412 | 0.23924 | 1.0058 | 0.078 | −0.4251 | 0.93215 |
| IFI6 \|TMEM107 \|LRRN3 \|PIP5K1B \|PRR3 \|SERPINE2 \|MSRB2 \|RPAP1 \|IFT27 \|STXBP4 | 2.78320484 | 0.26047 | 0.84807 | 0.11562 | −0.5704 | 0.93408 |
| ZWILCH \|CDH1 \|TIMP2 \|SLC46A3 \|SEMA4C \|E2F6 \|SMC2 \|PROK2 \|MRPL17 \|KIFC2 | 2.778031649 | 0.27202 | 0.78007 | 0.13694 | 1.06938 | 0.92086 |
| CDCA7L \|ASAP2 \|UAP1 \|TXLNG \|PARP16 \|WEE1 \|MTFP1 \|ARHGEF10 \|FPGT \|IGSF8 | 2.771056165 | 0.28681 | 0.73765 | 0.13138 | −0.2504 | 0.92379 |
| KBTBD8 \|CDC14B \|SLC6A8 \|NRIP1 \|HIST1H3E \|SPAG5 \|SCML1 \|PROCA1 \|ADNP2 \|ZNF37A | 2.764688262 | 0.27953 | 0.95571 | 0.06016 | −0.3482 | 0.92524 |
| TIAM2 \|PLEKHA8 \|BCORL1 \|ABCA3 \|TAS2R46 \|MIS12 \|IPO13 \|TMED1 \|IL18 \|ZNF26 | 2.75851052 | 0.26732 | 1.04176 | 0.09282 | −0.7437 | 0.94146 |
| ST20 \|NACC1 \|E4F1 \|TIMD4 \|WDR83 \|C9orf64 \|ZNF483 \|CYB561 \|KCNRG \|TXLNA | 2.754344298 | 0.2518 | 0.88775 | 0.14124 | 1.40487 | 0.93795 |
| UBE2V2 \|ZNF799 \|EBLN2 \|ENC1 \|LRRC41 \|GP9 \|SNRNP35 \|ZCCHC14 \|DHRS7B \|CDC7 | 2.74737755 | 0.26521 | 1.15234 | 0.04026 | −0.5428 | 0.93347 |
| DYNLT3 \|NFKBIE \|ARL13B \|GNMT \|FBXL6 \|TESK2 \|SLC19A1 \|ARL10 \|COL4A3 \|PTPRK | 2.741953933 | 0.25931 | 0.82286 | 0.12119 | 0.12412 | 0.90904 |
| TNFAIPSL2 \|APLF \|KCNQ2 \|LYRM2 \|RPGRIP1 \|TBC1D22B \|FAM169A \|SLC35C1 \|PDSS2 \|ADCK2 | 2.73442655 | 0.26442 | 0.9318 | 0.11421 | 0.10956 | 0.92804 |
| SWI5 \|ZNF555 \|ARV1 \|AVPR2 \|C19orf54 \|PYROXD2 \|PRPF40B \|TSTD2 \|GGT7 \|TRPT1 | 2.728759415 | 0.29167 | 0.7959 | 0.16745 | −0.6599 | 0.90344 |
| TAF1A \|MED19 \|RAB13 \|PMM1 \|CCDC30 \|RBMXL1 \|MSS51 \|HIST3H2A \|TSHR \|RFC3 | 2.723731525 | 0.26092 | 0.82691 | 0.07028 | −0.6658 | 0.94013 |
| LUZP1 \|ZBTB49 \|ATF3 \|PDE1B \|PIK3R6 \|CLDND2 \|TTC32 \|EPB41L5 \|ZNF382 \|HDDC3 | 2.717385204 | 0.26044 | 1.06882 | 0.08653 | −0.5402 | 0.92389 |
| C7orf31 \|BTLA \|OXSM \|ZNF250 \|TTLL1 \|ZNF90 \|FUK \|GMNN \|CYB561D2 \|LXN | 2.711399894 | 0.26979 | 0.91562 | 0.13746 | −0.4129 | 0.94224 |
| ARL4A \|RRP9 \|OCM \|MCM8 \|NT5C1B \|TLCD1 \|ANKRD54 \|BEX4 \|ISG15 \|ZNF565 | 2.702829318 | 0.26224 | 0.80831 | 0.15954 | 2.02393 | 0.93793 |
| DAPK2 \|KDELR3 \|L3MBTL4 \|C12orf43 \|ABCC2 \|C11orf84 \|DUSP7 \|TMEM144 \|YTHDF1 \|GRB10 | 2.693794287 | 0.22601 | 1.02098 | 0.13529 | −0.3962 | 0.92927 |
| CBY1 \|PELI3 \|PROCR \|CACNB2 \|ZNF473 \|SLC22A18 \|PGA4 \|SERAC1 \|ZNF514 \|APEX2 | 2.687925391 | 0.26746 | 0.70575 | 0.17394 | 0.85573 | 0.91553 |
| GPX7 \|HOOK1 \|KRT10 \|ALOX12 \|OR52K2 \|SCAF1 \|CEACAM1 \|PRICKLE1 \|C5 \|MTHFD1L | 2.682906502 | 0.22022 | 0.99429 | 0.07827 | 0.102 | 0.92658 |
| EXOSC5 \|CCDC127 \|FAM217B \|AFF2 \|NRBP2 \|ARAP3 \|SAMD4A \|SCCPDH \|ECHDC3 \|ANKDD1A | 2.677636684 | 0.27262 | 0.74883 | 0.22199 | 0.17142 | 0.91506 |
| NUDT2 \|USP46 \|ABLIM3 \|MAVS \|AAED1 \|SLC16A1 \|KEAP1 \|RFNG \|ASIC3 \|COQ4 | 2.670850754 | 0.25369 | 0.82872 | 0.12775 | 0.18664 | 0.91205 |
| LEAP2 \|ZNF671 \|NOD1 \|TSNARE1 \|SLC25A29 \|NAPB \|B4GALT4 \|LRRCC1 \|TBKBP1 \|MYL5 | 2.661959213 | 0.25687 | 0.96745 | 0.05958 | −0.8676 | 0.91219 |
| EARS2 \|KIF27 \|HOMER2 \|MDS2 \|MID2 \|RRAS \|RPUSD1 \|CACNA1D \|MFSD9 \|GKAP1 | 2.65510878 | 0.2612 | 0.81278 | 0.11073 | 0.21366 | 0.91978 |
| ZNF343 \|LYRM7 \|GPA33 \|SNAP47 \|ALDOC \|AGBL2 \|KLHL11 \|LGALS9C \|DISP1 \|ZNF135 | 2.645068175 | 0.25318 | 1.17402 | 0.06979 | 1.96366 | 0.93912 |
| KCNAB3 \|ANAPC11 \|TMEM216 \|CD274 \|TMPRSS13 \|SLC25A43 \|ABCC4 \|FCRL5 \|VEPH1 \|EPOR | 2.637111854 | 0.22678 | 1.10426 | 0.06027 | −0.5579 | 0.93208 |
| C2orf68 \|MRI1 \|TSPAN33 \|CHCHD6 \|MCF2L \|IPP \|SLC38A5 \|ELK1 \|COMMD8 \|RUSC1 | 2.62674838 | 0.26328 | 0.9043 | 0.19336 | −0.1816 | 0.92098 |
| BEND2 \|INPP5F \|RGS12 \|SLC2A11 \|TCEAL1 \|DGAT2 \|CLEC4C \|TRIM32 \|CCDC136 \|HRNR | 2.620161214 | 0.21965 | 1.04999 | 0.04502 | 0.21902 | 0.92685 |
| HCG27 \|WWC3 \|GUCY1A3 \|ATP1A3 \|DALRD3 \|DNAH14 \|TM7SF2 \|ADTRP \|HEBP1 \|TMEM159 | 2.616136637 | 0.26251 | 1.0679 | 0.07918 | −0.0693 | 0.927 |
| KIAA1324 \|C16orf87 \|ZNF571 \|LIX1L \|CACNB3 \|TFB2M \|UST \|FUT7 \|ATRIP \|THUMPD2 | 2.607389154 | 0.2642 | 0.77767 | 0.1607 | −0.8654 | 0.92099 |
| LIN52 \|ZNF180 \|TMEM220 \|MFHAS1 \|C12orf45 \|C2 \|PUS10 \|PAK4 \|DGKH \|EGR3 | 2.597909397 | 0.28295 | 0.84897 | 0.10393 | 1.34756 | 0.92446 |
| ANKS3 \|SLC31A2 \|CISD1 \|GSTCD \|ILVBL \|FLT3 \|RGS19 \|LUZP6 \|MS4A4A \|FAM86C1 | 2.590417029 | 0.25634 | 0.85065 | 0.16906 | −0.6952 | 0.93909 |
| C14orf80 \|KCNJ15 \|PLEKHO2 \|SFMBT1 \|SH3BP5L \|PRB2 \|NCAPG2 \|CHTF18 \|ARHGAP33 \|DNMBP | 2.58363109 | 0.2733 | 0.76401 | 0.08074 | −0.6042 | 0.92432 |
| MED31 \|PCSK6 \|GEMIN8 \|ZNF684 \|CEP76 \|ZNF222 \|AHSP \|ISCA2 \|GATM \|KHK | 2.57744812 | 0.27246 | 0.7788 | 0.2111 | −0.6363 | 0.93282 |
| ZNF345 \|CLIP2 \|SERTAD2 \|B3GNT2 \|GDE1 \|TRPM2 \|ADCY4 \|PDLIM7 \|FUT10 \|SNRNP48 | 2.571109825 | 0.2771 | 0.85495 | 0.17844 | −0.4303 | 0.91299 |
| XYLB \|SLC27A4 \|FOLR2 \|RTN4IP1 \|MYL6B \|BACE1 \|ABHD6 \|SCRN2 \|LAMB2 \|TUSC2 | 2.563949965 | 0.25839 | 0.94072 | 0.21913 | 0.27099 | 0.9186 |
| LRRC18 \|CAMSAP2 \|APOBEC3B \|GPM6B \|FAM72B \|LRRC57 \|DNAL1 \|EPHB4 \|ERI1 \|SMG8 | 2.557419158 | 0.24189 | 1.02716 | 0.09296 | 0.36118 | 0.93197 |
| RRP8 \|MAGEH1 \|PIGO \|TYW5 \|DGKQ \|APOBEC3H \|NCDN \|MANBAL \|ZC3B3 \|WWC2 | 2.549126256 | 0.27357 | 0.77226 | 0.12235 | −0.6967 | 0.9146 |
| FAM207A \|SPIRE1 \|MTMR8 \|GNAZ \|GMPR \|TP53IL1 \|TSHZ1 \|ZRANB3 \|MT1F \|COL19A1 | 2.542689199 | 0.25365 | 0.99112 | 0.11957 | 0.08576 | 0.91917 |
| MOAP1 \|CLN5 \|ZNF606 \|SERHL2 \|ZNF581 \|ZXDA \|ACADS \|INCA1 \|HAUS5 \|ERV3-1 | 2.534745929 | 0.28354 | 0.91069 | 0.15136 | −0.4134 | 0.9199 |
| SMAGP \|GTPBP3 \|ATP13A4 \|PACSIN1 \|FCGR3B \|ZSCAN18 \|TRIM62 \|RBPMS \|BCAT2 \|ZC3H12D | 2.529535542 | 0.21451 | 1.2441 | 0.01292 | −0.2376 | 0.91931 |
| FAM103A1 \|FAM46C \|SLC35D1 \|SLC17A9 \|LIN9 \|CEBPA \|USP35 \|LIPE \|PHEX \|HECA | 2.523236414 | 0.26078 | 1.01519 | 0.08721 | −0.4693 | 0.91904 |
| CACFD1 \|ZMYM3 \|FAM216A \|VSIG4 \|ZNF208 \|ZNF569 \|AASS \|CCR9 \|AMMECR1L \|FHL3 | 2.518109576 | 0.23419 | 0.99396 | 0.09592 | −0.354 | 0.93381 |
| ANKRD20A4 \|CRYBA1 \|P2RY14 \|CDRT4 \|TMEM39B \|C14orf79 \|RSPB3 \|MLYCD \|TRMT5 \|C15orf41 | 2.510986265 | 0.24287 | 1.04398 | 0.14378 | −1.0316 | 0.93472 |
| PPP1R3F \|NEFH \|FANCF \|EXD2 \|AACS \|MXD3 \|TCEA2 \|CCNJ \|NR1H3 \|SEMA4F | 2.502064632 | 0.26878 | 0.8262 | 0.20013 | 1.42235 | 0.91569 |
| SIRT6 \|MYOM1 \|UCK2 \|MCF2L2 \|ARSD \|PDCD2L \|PLGRKT \|TRMT10C \|ABHD12B \|GCC1 | 2.494466031 | 0.27226 | 0.87603 | 0.1782 | 0.74882 | 0.93392 |
| ACVR2B \|MMP23B \|PPARD \|PUS7 \|ZDHHC12 \|CTSF \|VPS18 \|TRAF1 \|ACOT13 \|CHCHD5 | 2.488829613 | 0.27522 | 0.71044 | 0.21593 | −0.3875 | 0.90987 |
| C2CD2 \|TRMT10A \|PATZ1 \|SLC46A2 \|GOLGA6L6 \|CTHRC1 \|ZNF200 \|APOL6 \|BNIP1 \|DDR1 | 2.480515738 | 0.2581 | 1.15191 | 0.13284 | −0.3684 | 0.91661 |
| GHDC \|TMEM80 \|LAG3 \|DDIT3 \|ABCD2 \|NCKIPSD \|NTMT1 \|SGCE \|COX7A1 \|DNAAF2 | 2.474100301 | 0.24896 | 1.06378 | −0.0055 | −0.7832 | 0.91323 |
| ZEP30 \|NPM3 \|MMP17 \|TSR2 \|PBX4 \|CD180 \|GLB1L \|TMEM117 \|BPGM \|RPUSD4 | 2.467778611 | 0.26413 | 0.82695 | 0.1684 | 0.0942 | 0.93219 |
| WDR81 \|IL17RC \|TSPAN9 \|IFT140 \|CD200 \|STARD5 \|ANK1 \|TMED7-TICAM2 \|WDR54 \|RHOB | 2.455922445 | 0.26801 | 0.79906 | 0.14033 | −0.6906 | 0.91053 |
| DVL1 \|ATL1 \|SPEG \|PLCXD2 \|LRRC8A \|LIME1 \|SLC4A4 \|A1BG \|MAFK \|NRBF2 | 2.447424951 | 0.24238 | 0.96556 | 0.0692 | −0.2405 | 0.90338 |
| PER3 \|NUDCD2 \|MAP3K13 \|DUS4L \|AIFM3 \|DPPA4 \|PPARGC1B \|MRPL12 \|ST6GALNAC1 \|ZNF443 | 2.440020116 | 0.24049 | 0.85595 | 0.1804 | −0.1545 | 0.92889 |
| NRG4 \|LBX2 \|ZNF549 \|SIRT5 \|HMGN4 \|NIPAL4 \|ECT2 \|ZNF34 \|WDR35 \|NMNAT1 | 2.430991681 | 0.26291 | 0.94513 | 0.06154 | −0.6845 | 0.93541 |
| PHKG1 \|PDE6C \|GNA15 \|LRRC23 \|AVIL \|NHSL1 \|TMEM41A \|PLEKHG1 \|PRICKLE3 \|ENTPD7 | 2.424300143 | 0.23462 | 1.03962 | 0.10724 | 0.12622 | 0.92881 |
| HOPX \|ZNF365 \|DLG5 \|SRR \|APOM \|LGR6 \|NKIRAS1 \|CASS4 \|ZNF613 \|PEX11B | 2.417472996 | 0.25033 | 0.95697 | 0.02298 | −0.3056 | 0.92089 |
| SUMO4 \|ZBTB22 \|RRAGC \|SERPINH1 \|SYNM \|PADI2 \|WASL \|LSM3 \|CSRNP2 \|DSN1 | 2.403775038 | 0.2799 | 0.93623 | 0.08724 | 0.12769 | 0.92339 |
| ALKBH7 \|DAX58 \|C11orf74 \|TM9SF1 \|ZNF324B \|CRY1 \|RFX2 \|TGM1 \|ZKSCAN4 \|GPX3 | 2.396383632 | 0.26437 | 1.04422 | 0.06303 | −0.12 | 0.9203 |
| PDE6G \|OXER1 \|ITGB4 \|ZNF22 \|TSR3 \|TKTL1 \|CENPP \|BUB1B \|GMPPB \|TMEM160 | 2.390472827 | 0.25692 | 0.86069 | 0.11468 | 0.23308 | 0.91358 |
| FAM219A \|LMLN \|SAP25 \|ZNF546 \|SRD5A1 \|ZNF717 \|DBNDD2 \|SPIN2A \|PHOSPHO1 \|TMEM128 | 2.381888446 | 0.26557 | 0.88147 | 0.12801 | −0.1475 | 0.91668 |
| DUSP5 \|FBXO24 \|RNF175 \|BEND5 \|GRAMD1C \|NUDCD1 \|METTL4 \|C1orf50 \|MORN1 \|ELAC1 | 2.373683163 | 0.26216 | 0.85704 | 0.06758 | −0.5527 | 0.92362 |
| SLC7A1 \|BRAT1 \|CYBSD1 \|TMEM185B \|OASL \|TMC4 \|LIM2 \|PRUNE2 \|ZBTB4 \|LIPT1 | 2.366417461 | 0.26097 | 0.88579 | 0.13489 | −1.0495 | 0.91342 |
| LOXHD1 \|PUS3 \|ZNF500 \|WDFY2 \|ZNF133 \|CTU2 \|TRMT12 \|CKMT2 \|OLFM2 \|POU6F1 | 2.358592277 | 0.25324 | 0.92991 | 0.21552 | −0.6376 | 0.91993 |
| GPR141 \|PPP1R37 \|RECK \|LTC4S \|NT5DC2 \|STK35 \|YES1 \|STX1A \|SNX16 \|SLC43A1 | 2.346749846 | 0.26111 | 0.9898 | 0.07666 | −1.0069 | 0.91968 |
| SNX22 \|PPFIBP1 \|SELP \|NRP2 \|MZF1 \|TAX1BP3 \|ZNF32 \|NEDD8-MDP1 \|IRGQ \|KPTN | 2.337801956 | 0.2694 | 0.84048 | 0.11573 | −0.8935 | 0.92736 |
| MIBP \|OTUD1 \|TSPY4 \|CCRL2 \|PPARGC1A \|SPSB2 \|LHB \|TOR1AIP2 \|BEAN1 \|FN3K | 2.329127891 | 0.24169 | 1.11969 | 0.07229 | −0.4905 | 0.91721 |
| FAM210A \|TP53RK \|EXOC8 \|FAM199X \|VPS37C \|ZNF14 \|C1orf35 \|DYNC2H1 \|NXT2 \|PPFIA4 | 2.316866627 | 0.26501 | 0.97074 | 0.019 | −0.1057 | 0.93651 |
| NKAP \|CMTM2 \|USP54 \|GP1BB \|ZSWIM1 \|H2AFX \|MSR1 \|ME3 \|SLC29A2 \|TRIM11 | 2.304163419 | 0.27058 | 1.07372 | 0.01591 | −0.6429 | 0.92164 |
| BRCA2 \|TMEM161A \|PLA2G4C \|DYRK1B \|MINPP1 \|ZNF669 \|PKIA \|CCDC138 \|ABI3BP \|RETN | 2.294509785 | 0.25342 | 0.9557 | 0.12014 | −0.6281 | 0.93438 |
| SUSD4 \|SLC4A10 \|GPR162 \|AMY2A \|TIMM8B \|LGALS3BP \|STS \|GNAL \|SUV39H2 \|CA1 | 2.287258475 | 0.19543 | 1.09513 | 0.0459 | −0.5934 | 0.93084 |
| GSPT2 \|IFIT3 \|C12orf73 \|TEF \|ARMCX6 \|CLDN20 \|HBG1 \|ZNF616 \|LETM1 \|MRPL14 | 2.278043446 | 0.26187 | 0.79144 | 0.15437 | −0.611 | 0.93266 |
| TEN1 \|NFIX \|ELL3 \|KIR2DL4 \|IMP3 \|ENDOV \|RANBP6 \|VWA5A \|RGS1 \|KLF8 | 2.271814388 | 0.25178 | 0.98353 | 0.14811 | −0.3846 | 0.93438 |
| ICOSLG \|LRRC69 \|OPN1SW \|SMAD7 \|CXorf38 \|ZKSCAN3 \|MAPK6 \|PLXNA1 \|ZNF821 \|CREBL2 | 2.260156774 | 0.23049 | 1.09704 | 0.10843 | −0.5599 | 0.92034 |
| BFSP2 \|CCHCR1 \|B3GNT8 \|RSC1A1 \|EFCAB11 \|SERPINI2 \|SYT17 \|ZNF428 \|TOR2A \|CTH | 2.25316361 | 0.23516 | 1.13656 | 0.02191 | 1.32022 | 0.93289 |
| C14orf93 \|VPS4A \|SLC26A2 \|DSTYK \|DNAJC27 \|DHCR7 \|LPAR2 \|ANGEL1 \|TULP2 \|ZXDB | 2.240906296 | 0.2864 | 0.83498 | 0.12423 | −0.3697 | 0.92266 |
| ZIK1 \|MIPEP \|CLCN4 \|MFSD2A \|AKR1C1 \|NLGN4Y \|SH3BGR \|CALB1 \|PLK2 \|SASS6 | 2.232214862 | 0.23667 | 0.96441 | 0.15205 | 0.02065 | 0.93669 |
| MLLT1 \|MPP5 \|BRF2 \|ENPP4 \|NLE1 \|ERBB3 \|NUDT17 \|HDAC11 \|MED18 \|C16or91 | 2.222741448 | 0.26848 | 0.83147 | 0.14382 | −0.4639 | 0.90974 |
| PLGLB2 \|RASGEF1A \|EPAS1 \|IRS2 \|SLC1A5 \|NUDT13 \|MMP25 \|WDSUB1 \|CDKN2C \|CCDC106 | 2.211336746 | 0.25874 | 1.05674 | 0.01327 | 0.55461 | 0.91001 |
| HLX \|ANXA8L1 \|DEGS2 \|MSMO1 \|SNX21 \|UACA \|ST6GALNAC3 \|SORD \|GNAO1 \|B3GNT4 | 2.204159366 | 0.25729 | 0.92637 | 0.15485 | 3.23122 | 0.91997 |
| HIGD1B \|ENAH \|IPPK \|PARPBP \|FMNL2 \|TMEM143 \|KBTBD4 \|FAM221A \|OGFOD2 \|TCL1A | 2.197243336 | 0.2465 | 0.97015 | 0.08854 | 0.2503 | 0.92726 |
| MORC4 \|PQLC2 \|AP5B1 \|KCNIP2 \|SLC6A12 \|NINJ1 \|RSAD2 \|PC \|SYNJ2BP \|TRIM17 | 2.188148536 | 0.25191 | 0.9801 | 0.09604 | 0.12522 | 0.91645 |
| ARRDC4 \|PSMG4 \|PFN2 \|HSPA13 \|ZNF230 \|VCPIP1 \|KCNIP4 \|SLC39A1 \|RBM45 \|CYSLTR1 | 2.180294685 | 0.26669 | 0.55206 | 0.22424 | −0.6204 | 0.93044 |
| KCNQ5 \|GAS8 \|CRIPT \|AURKA \|MUSTN1 \|GLI1 \|TPST1 \|PVR \|TRIM58 \|JRKL | 2.170907681 | 0.26525 | 0.83929 | 0.14119 | 0.50876 | 0.92191 |
| PMVK \|XXYLT1 \|PHYHD1 \|METAP1D \|CENPO \|C1QB \|WDR78 \|SLC1A4 \|TMEM120B \|SAV1 | 2.159689346 | 0.2632 | 0.85533 | 0.12203 | −0.5248 | 0.93023 |
| NFKBIB \|ZNF543 \|PIWIL4 \|NRCAM \|GAGE1 \|NAB2 \|KRBA1 \|ZNF627 \|USP18 \|CHCHD1 | 2.150640734 | 0.26486 | 0.74122 | 0.16554 | −0.6553 | 0.93242 |
| TMEM42 \|MED7 \|ADAMTS6 \|FOXP3 \|ZNF707 \|RTN2 \|CDK3 \|BTBD19 \|MIER2 \|MSRB3 | 2.14298333 | 0.27584 | 1.00547 | 0.15631 | 1.37292 | 0.91821 |
| ZNF618 \|BPI \|ARL16 \|PPM1N \|MICALCL \|C12orf42 \|GIPC1 \|PAQR8 \|ZNF683 \|SLC12A5 | 2.135734722 | 0.24512 | 1.07029 | 0.02374 | 0.23703 | 0.91939 |
| AFAP1 \|TPRG1L \|INPP5E \|GLIS3 \|GIPR \|WBP1L \|GRPEL2 \|CHEK1 \|UBE2W \|LGALS12 | 2.127609885 | 0.26761 | 0.91483 | 0.05432 | −0.0951 | 0.92111 |
| GPAM \|COPG2 \|SLC31A1 \|SERPIND1 \|EFCAB7 \|KLHL17 \|HSD17B3 \|GRM7 \|SYT11 \|GTF3C4 | 2.122683047 | 0.24388 | 1.00141 | 0.05649 | −0.4331 | 0.93047 |
| ACTR5 \|LAIR2 \|RBM15B \|KLF5 \|MGAM \|ZHX3 \|SOCS4 \|ZNF558 \|EDARADD \|ORM1 | 2.115560914 | 0.22717 | 0.97497 | 0.09533 | −0.5027 | 0.93695 |
| TMEM150B \|NRM \|CYB5RL \|HIST3H2BB \|BNIPL \|TIMELESS \|LZTS2 \|BAHD1 \|EGF \|TUT1 | 2.109834296 | 0.23719 | 1.00779 | 0.0383 | −0.6704 | 0.92796 |
| C6orf163 \|ZNF625 \|MTHFS \|ACSM1 \|EML5 \|DPY19L2 \|TWSG1 \|LONRF3 \|ADAL \|MKI67 | 2.102119176 | 0.23526 | 0.9383 | 0.07861 | −0.8042 | 0.93602 |
| XKR8 \|FAM90A1 \|CCDC17 \|PPIL1 \|SMOX \|TCTA \|CLTCL1 \|SIGLEC6 \|FBXL19 \|EXD3 | 2.093408842 | 0.23861 | 1.06155 | 0.12738 | 1.53593 | 0.90828 |
| TSPO2 \|MYOT \|WFS1 \|RXFP4 \|CBR1 \|ANKRD39 \|WDHD1 \|CERS6 \|PTCD1 \|RAB3A | 2.087412059 | 0.25231 | 0.8273 | 0.19689 | −0.6817 | 0.92559 |
| AP1S3 \|FAM214B \|VSIG2 \|C20orf27 \|TMEM25 \|CENPV \|CYSTM1 \|GPR132 \|TBC1D12 \|MSANTD4 | 2.082347692 | 0.25656 | 0.96924 | 0.05393 | 0.52748 | 0.90999 |
| NIPSNAP3B \|MRPL55 \|RILP \|ALKBH2 \|ZSCAN21 \|ZCCHC4 \|CA11 \|ADORA2A \|RGPD3 \|EBPL | 2.072687429 | 0.26465 | 0.97739 | 0.09687 | 0.80736 | 0.91427 |
| NKRF \|WDR17 \|GPR75 \|KALRN \|OVGP1 \|BCL2 \|FAM81B \|PRSS12 \|CENPE \|EPB42 | 2.063282645 | 0.20306 | 1.09833 | 0.05712 | 0.89083 | 0.9395 |
| EPHB2 \|ZNF583 \|FMO4 \|ACOT7 \|IL1R1 \|PXMP4 \|DNASE1L3 \|CCDC86 \|MPL \|IQCK | 2.051564828 | 0.2246 | 1.03002 | 0.076 | 0.10429 | 0.92394 |
| GDPGP1 \|KLC2 \|TJP3 \|C15orf61 \|CCR4 \|CORO7-PAM16 \|FAM122A \|SKA3 \|RAB40B \|ABT1 | 2.044542104 | 0.27314 | 0.87219 | 0.07742 | −0.1567 | 0.92538 |
| PLCD4 \|ZNF57 \|PRSS8 \|ZNF20 \|GCNT7 \|ARHGAP11A \|RNF207 \|CLIC3 \|PCBP4 \|TMOD4 | 2.038089349 | 0.21742 | 1.09669 | 0.04147 | 2.08555 | 0.91302 |
| DOLPP1 \|GLB1L2 \|MB21D1 \|ZNF467 \|HYAL1 \|FEM1C \|NRP1 \|OSBP2 \|FNDC9 \|ERMAP | 2.03129861 | 0.24397 | 0.76128 | 0.17868 | 1.65655 | 0.91352 |
| ZNF620 \|RXFP2 \|GPT \|SOCS6 \|ZDHHC23 \|CBS \|KIF3B \|NUBP2 \|ZNF260 \|MACROD2 | 2.022906265 | 0.24364 | 0.94414 | 0.07964 | 0.25667 | 0.92508 |
| TMEM5 \|TMEM79 \|CD302 \|DIP2C \|SYCE1L \|OMG \|XCL1 \|SLFN14 \|MOSPD1 \|SLC16A13 | 2.01589063 | 0.21364 | 0.98989 | 0.04296 | 0.13907 | 0.93886 |
| ARR3 \|CTPS2 \|SLC35A4 \|UBE3D \|TRAF3IP2 \|LYRM4 \|TMEM182 \|KLHL36 \|DHRS13 \|RMI1 | 2.008765875 | 0.27465 | 0.88752 | 0.09811 | −0.8182 | 0.93902 |
| SPTBN2 \|ABHD13 \|PAX8 \|RWDD2B \|MRPS26 \|CMTM5 \|SLC52A2 \|GH1 \|NR1D1 \|EIF4EBP2 | 1.996223367 | 0.24832 | 1.09879 | 0.06126 | 0.88383 | 0.90803 |
| ST6GALNAC2 \|SERPINI1 \|ZNF251 \|NUDT16L1 \|SYCP3 \|C17orf107 \|SERTAD3 \|NRGN \|LY6G5B \|ARSK | 1.987659109 | 0.28187 | 0.89061 | 0.13322 | −1.0092 | 0.91813 |
| CCL2 \|ANKRD55 \|SDF2L1 \|SOCS2 \|ZFYVE28 \|ST6GALNAC4 \|B3GALT4 \|EPS8 \|OSMR \|FOXP4 | 1.981400747 | 0.23442 | 0.86284 | 0.09451 | −0.2805 | 0.92441 |
| CYSLTR2 \|SPNS3 \|TRIAP1 \|DGKE \|ATAD5 \|RNF185 \|MRVI1 \|PRADC1 \|MACROD1 \|CCSAP | 1.974482012 | 0.25034 | 0.92547 | 0.1538 | −0.612 | 0.92601 |
| SLC41A1 \|MYCBP \|CASZ1 \|ATP1B1 \|WIZ \|TNK1 \|FICD \|STEAP4 \|C14orf178 \|SLC38A7 | 1.966537213 | 0.23745 | 1.02183 | 0.06196 | 0.14439 | 0.91608 |
| MSANTD2 \|PCOLCE \|TMEM229B \|VNN1 \|KIAA1524 \|RASSF7 \|PTGR2 \|DET1 \|METTL12 \|RCAN1 | 1.960623466 | 0.27181 | 0.87369 | 0.08391 | −0.586 | 0.92571 |
| LY6G6F \|PTPRS \|OR52N4 \|OCLN \|GINS4 \|SCAMP5 \|ZNF341 \|MAFB \|NTHL1 \|LLGL1 | 1.952101258 | 0.26071 | 0.76101 | 0.14026 | 2.00215 | 0.91079 |
| PEX12 \|ZSCAN12 \|JAGN1 \|AGRN \|CALD1 \|BCO2 \|NEFM \|ZNF681 \|CNTNAP1 \|CETN2 | 1.944490729 | 0.24452 | 1.07658 | 0.13242 | −0.0279 | 0.9362 |
| ZNF726 \|ANGPTL1 \|APCDD1 \|CNIH2 \|ZDHHC24 \|EML6 \|SETD9 \|ZC4H2 \|TRIM16L \|LHFPL2 | 1.934239767 | 0.2463 | 1.038 | 0.07911 | −0.7257 | 0.92661 |
| FEM1A \|FAM212B \|AMY1C \|TDRKH \|DCST2 \|TIPIN \|B4GALT6 \|ATG14 \|RNF215 \|DDX28 | 1.923264624 | 0.26087 | 0.93179 | 0.18236 | −0.8831 | 0.91938 |
| PRB2 \|ABCB6 \|TNF \|JMY \|PAOX \|ZNF469 \|GPR137 \|NID1 \|NAV1 \|EPN2 | 1.911412338 | 0.23785 | 0.85867 | 0.06105 | 2.50399 | 0.89685 |
| DBF4B \|RBM43 \|S1PR5 \|C8orf44 \|WDR25 \|GLCE \|E2F3 \|SLC25A42 \|RAB19 \|PMAIP1 | 1.901164612 | 0.28025 | 0.94073 | 0.10627 | −0.3719 | 0.92465 |
| ZSCAN2 \|ABHD14A \|HMGB3 \|RUSC2 \|METTL1 \|THAP9 \|MYO1A \|SNAPC2 \|ARMCX5 \|EIF4EBP3 | 1.892887931 | 0.26761 | 1.02598 | 0.10552 | 0.1706L | 0.92842 |
| TRIM3 \|UBE2S \|NFKBIL1 \|ZNF793 \|SCGB3A2 \|SOGA1 \|TGIF2 \|HOXB3 \|TMUB1 \|ZNF182 | 1.886562807 | 0.23033 | 1.07575 | 0.05038 | 0.01373 | 0.92502 |
| MTSS1L \|ZNF442 \|ANGPTL4 \|FANCB \|CPA5 \|WDR89 \|ASB14 \|ZNF10 \|TMEM64 \|LRR1 | 1.878415922 | 0.24455 | 0.93087 | 0.08592 | 0.36257 | 0.9366 |
| SRXN1 \|LIN78 \|SGPP1 \|TSPAN13 \|GUCA1B \|ZNF749 \|GAMT \|ADCK5 \|ZNF232 \|PLK1 | 1.868759129 | 0.25558 | 0.82418 | 0.10792 | −0.5437 | 0.91977 |
| ANXA8 \|HSDL1 \|ZNF786 \|CHCHD4 \|ENHO \|ZNF2 \|PGAM4 \|NUDT14 \|PMP22 \|ZNF777 | 1.85805614 | 0.24468 | 0.79683 | 0.06718 | 1.03784 | 0.91059 |
| MDK \|SPAG8 \|DMC1 \|ARMC7 \|AMMECR1 \|TLR3 \|URB1 \|TMIGD2 \|CAB39L \|MCHR1 | 1.851695878 | 0.2521 | 0.98726 | 0.14763 | 0.17429 | 0.92812 |
| RAB11FIP5 \|RBM11 \|FAM200B \|HSPG2 \|RRAGD \|FAM196B \|PRKAB2 \|PTPN13 \|PLXNA4 \|NUF2 | 1.839595403 | 0.25156 | 0.76803 | 0.13891 | −0.6306 | 0.93148 |
| SLC38A11 \|FAM120C \|PLA2G15 \|PBX1 \|ZNF605 \|C6orf47 \|LCA5L \|TNFRSF10D \|ELMO3 \|HOMER1 | 1.830735595 | 0.25923 | 0.88161 | 0.14995 | −0.4314 | 0.92327 |
| MAP3K6 \|DES \|DUX4L7 \|FAM46A \|SLC22A23 \|ESAM \|C10orf88 \|FAM151B \|NUAK2 \|CEP72 | 1.823421236 | 0.28188 | 0.96395 | 0.17167 | 0.6444 | 0.91055 |
| PLIN4 \|CACNA1F \|NCKAP5L \|PTRH1 \|RIMBP3 \|C6orf201 \|COMMD5 \|ANGPT2 \|IPMK \|FRS3 | 1.818133688 | 0.21934 | 1.02498 | 0.04568 | −0.9394 | 0.90841 |
| CHST12 \|CDHR1 \|TTC24 \|RNF24 \|RBM14-RBM4 \|C1orf159 \|TLR10 \|GAS6 \|CCDC78 \|AURKC | 1.811041902 | 0.22755 | 1.07785 | 0.00714 | −0.1607 | 0.9198 |
| ZFP1 \|RARG \|OCEL1 \|ST8SIA6 \|GLRX5 \|SCN9A \|CDH26 \|CLCN2 \|EPS8L1 \|ARL6 | 1.799256906 | 0.24358 | 0.92084 | 0.05536 | −0.1164 | 0.91631 |
| SAMD10 \|CLDN7 \|SDK2 \|ZNF596 \|DPM2 \|RFX1 \|USP49 \|PARD3 \|TLE1 \|FAM24B | 1.787029518 | 0.25914 | 0.91881 | 0.03044 | −0.4317 | 0.90395 |
| ZBTB34 \|PLEKHJ1 \|DPH3 \|AHNAK2 \|ZNF764 \|TAS2R60 \|KDELC2 \|TCEANC2 \|DNAJB5 \|ZNF518B | 1.775819211 | 0.26464 | 0.8703 | 0.07204 | 0.11673 | 0.92357 |
| LRRC70 \|TNFSF12-TNFSF13 \|C5orf63 \|RNASE3 \|AMIGO1 \|NLRP7 \|HTR2B \|ZNF792 \|CDC42BPA \|PDIK1L | 1.766215091 | 0.2371 | 1.06469 | 0.02189 | 0.18264 | 0.93445 |
| PPAT \|GREB1L \|GGCT \|RFXAP \|TTL \|LOXL4 \|CCL28 \|RCN3 \|C12orf76 \|SRGAP3 | 1.753032807 | 0.23079 | 1.02888 | 0.04179 | 0.24929 | 0.93184 |
| PPL \|CCNJL \|TNS1 \|FAM213A \|SH3PXD2A \|ZDHHC9 \|LAMP3 \|WDR24 \|COQ10A \|TSHZ2 | 1.740903618 | 0.23349 | 1.04854 | 0.07447 | −0.9358 | 0.91526 |
| TTLL12 \|ZNF689 \|GAGE10 \|GEMIN7 \|TMCC3 \|CTRL \|GCNT1 \|KIT \|ZNF16 \|EXOSC4 | 1.734277158 | 0.241 | 0.92279 | 0.13038 | −0.5031 | 0.92127 |
| SNX32 \|DENND5B \|RGS6 \|IQCC \|LPAR1 \|TCEANC \|SH3RF1 \|ITGB1BP2 \|SPATA6L \|LAPTM4B | 1.727784818 | 0.25527 | 1.00928 | 0.05364 | −0.8296 | 0.92618 |
| ZNF530 \|HIRIP3 \|FAM106A \|GNA12 \|CCNE2 \|ISOC2 \|HDX \|RAPGEFL1 \|DMWD \|TMEM115 | 1.718029511 | 0.28399 | 0.79892 | 0.18235 | 0.47341 | 0.91789 |
| TRPC1 \|CACNA1I \|MORN3 \|PRDM11 \|BCL2L15 \|ZMYND19 \|AMACR \|C6orf120 \|CYB561D1 \|HOXA2 | 1.711243794 | 0.22993 | 0.85233 | 0.0645 | 0.36977 | 0.92268 |
| MYO5B \|STIL \|ZNF221 \|ACYP2 \|MFSD7 \|TIMM22 \|RAB6B \|ZNF30 \|CDHR3 \|ZNF48 | 1.705254945 | 0.23594 | 1.08771 | 0.0659 | −1.0121 | 0.92341 |
| ZNF408 \|TMEM65 \|RNF26 \|FSTL1 \|MMP28 \|GLDC \|TNIP3 \|COL6A1 \|RELL2 \|RYR3 | 1.699122542 | 0.24517 | 0.90281 | 0.07295 | 0.06914 | 0.91777 |
| MOK \|CYP2F1 \|TNFRSF11A \|PIGC \|SIPA1L21 \|IDO1 \|RHPN1 \|LRRC42 \|SH3D19 \|GAR1 | 1.6920892 | 0.24044 | 1.07382 | 0.08554 | −0.9012 | 0.93888 |
| ZNF593 \|KCTD15 \|FKRP \|C1GALT1C1 \|SAPCD1 \|LANCL2 \|FHAD1 \|NEIL2 \|FAM227B \|SLC35G5 | 1.687886396 | 0.24853 | 0.8775 | 0.15265 | −0.57 | 0.91945 |
| SLCO4C1 \|HIVEP3 \|CKS2 \|SH3BGRL2 \|RAB43 \|ICA1 \|MAPK8IP1 \|CTRC \|MRPL50 \|RPH3A | 1.679539451 | 0.22531 | 0.92828 | 0.04219 | 0.47375 | 0.92708 |
| ZNF850 \|ANKRD37 \|MXRA7 \|OTUD7B \|NQO1 \|POLR3K \|DEFA3 \|DCLRE1B \|SAMD12 \|KCNMB3 | 1.669829816 | 0.24858 | 0.82866 | 0.11573 | 0.8507 | 0.93474 |
| TAS2R19 \|DBX32 \|GYPA \|GEMIN6 \|EPS8L2 \|DUSP8 \|MMGT1 \|CPAMD8 \|ENDOG \|CLCF1 | 1.662507278 | 0.2248 | 0.96833 | 0.04199 | 0.02061 | 0.92339 |
| IZUMO4 \|GSTA4 \|GPR68 \|DFNAS \|FAM173A \|ZNF750 \|GEMIN4 \|B9D1 \|ANXA2R \|ZNF713 | 1.653661641 | 0.24436 | 1.14579 | 0.0330\| | −0.5478 | 0.92325 |
| SOCS5 \|TMEM14A \|RAD51AP1 \|CLEC4F \|ASB6 \|SMUG1 \|C1S \|ST8SIA1 \|POLE2 \|SEPHS2 | 1.645849574 | 0.25751 | 0.91251 | 0.12488 | −0.6774 | 0.93115 |
| NPHP4 \|C2orf69 \|CDT1 \|TBC1D24 \|SWSAP1 \|ZNF831 \|TRPV1 \|FAM20B \|NBEA \|CCDC157 | 1.63796526 | 0.25886 | 0.87464 | 0.13692 | 0.40589 | 0.91627 |
| COL6A3 \|STAR \|ALKBH1 \|SAYSD1 \|PDZD2 \|DHCR24 \|FSBP \|CD248 \|PALM3 \|PSKH1 | 1.630528918 | 0.23479 | 0.83591 | 0.15728 | −0.592 | 0.92853 |
| DIO1 \|GOLGA6A \|PGAP1 \|TP53INP1 \|PLAGL2 \|KCNMA1 \|ZFP28 \|SGCB \|DNA2 \|C19orf71 | 1.623854351 | 0.24219 | 1.04332 | 0.02177 | 1.0212 | 0.91352 |
| APBB2 \|BRIP1 \|SULT1A2 \|MRPS14 \|MPST \|MED26 \|LENG9 \|KIF23 \|THAP2 \|ADPRH | 1.617962393 | 0.27033 | 0.89859 | 0.04912 | −0.4306 | 0.92321 |
| AMDHD1 \|FEN1 \|MANEAL \|PLN \|RASL11A \|KCTD12 \|AMOT \|ADI1 \|HYAL2 \|PLTP | 1.609818622 | 0.26071 | 1.0932 | 0.02494 | 2.03391 | 0.92552 |
| MFSD3 \|RAB33A \|FAHD1 \|ITPRIP \|B4GALNT3 \|KIF3C \|ZNF785 \|SLC13A4 \|RILPL1 \|MBOAT4 | 1.603031725 | 0.24457 | 0.98698 | 0.08165 | −0.8732 | 0.91379 |
| CENPQ \|CA14 \|GAN \|TTLL11 \|CES4A \|DCLRE1A \|HS6ST1 \|CTDSPL \|CCDC102B \|SCN3A | 1.598346951 | 0.23477 | 1.08125 | 0.05733 | 0.45346 | 0.93384 |
| GPR152 \|ZNF519 \|ZNF19 \|KLHL21 \|C20orf96 \|ZFYVE9 \|TLE2 \|C1QTNF3 \|CAMKK1 \|PDE8B | 1.590376604 | 0.22471 | 1.01255 | 0.05709 | −0.6562 | 0.92147 |
| TFPI \|COPZ2 \|ESRRA \|DNAJC5B \|ZFP3 \|THRB \|PPP1R3B \|MID1IP1 \|WBP2NL \|ITPKC | 1.583670755 | 0.26326 | 1.06373 | 0.07748 | −0.7103 | 0.923 |
| CC2D2B \|PPP1R16A \|UPB1 \|ZNF485 \|PANX1 \|AEN \|ANGPT1 \|ELOVL6 \|SLC23A1 \|TMEM169 | 1.577773862 | 0.22823 | 1.10803 | 0.04238 | −0.2882 | 0.92633 |
| OTUD3 \|CAPN12 \|ZNF304 \|ZFP82 \|ITGA3 \|TUBA3D \|FOXK1 \|RNF32 \|NR6A1 \|ZNF646 | 1.56989302 | 0.24749 | 0.95144 | 0.04487 | −0.4416 | 0.91562 |
| CCNB1 \|CKLF-CMTM1 \|ZBTB5 \|ARHGAP42 \|UPK3B \|CKMT1A \|EFHC2 \|UBOX5 \|CHST14 \|HOXB2 | 1.564404139 | 0.23506 | 0.8182 | 0.16889 | −0.428 | 0.91982 |
| ANKRD20A3 \|CYP2A6 \|ZNF35 \|LTA \|RPP25L \|MARK1 \|FOXM1 \|GAGE12J \|PSTK \|SIT1 | 1.55737636 | 0.23042 | 1.07427 | 0.06032 | −0.756 | 0.92108 |
| NUDT15 \|MUC1 \|FAM136A \|C1QL3 \|TAF5 \|ADAM23 \|DPF3 \|NPPA \|CHIC1 \|WDR62 | 1.548180141 | 0.26246 | 0.8955 | 0.1567 | −0.2557 | 0.92329 |
| ZNF512B \|PRR4 \|CLEC5A \|ASTN2 \|ZNF132 \|CD209 \|C11orf71 \|FBXO46 \|DPCR1 \|RYR1 | 1.538674715 | 0.22956 | 0.95089 | 0.07135 | −0.7841 | 0.91896 |
| NFIB \|MTCP1 \|CXorf21 \|PEX10 \|APOBEC2 \|MOB3B \|PCDHGC3 \|HAGHL \|MAP9 \|CYP3A4 | 1.52546833 | 0.22032 | 1.19285 | 0.02657 | −0.914 | 0.93049 |
| ZNF768 \|CRHBP \|RAB26 \|AP3B2 \|SATB2 \|MED9 \|PIPOX \|CENPW \|SPTB \|OR52K1 | 1.515401744 | 0.21676 | 0.9666 | 0.08631 | 3.08722 | 0.92412 |
| FXYD7 \|ZNF668 \|ADPRHL2 \|DNAJC18 \|CENPN \|FIGNL1 \|IL31RA \|CKB \|ZNF677 \|ZNF574 | 1.505166485 | 0.24085 | 1.02445 | 0.07385 | 0.01998 | 0.9182 |
| TRIM74 \|MUC4 \|BAD \|FAM114A1 \|MYO15A \|CELA2B \|YRDC \|ZNF446 \|KBTBD7 \|HUS1B | 1.491227648 | 0.20666 | 0.93856 | 0.09971 | −0.1341 | 0.91305 |
| LRP6 \|PGM5 \|CYP3A7 \|ERI2 \|IKBIP \|UBXN2A \|ICAM4 \|LGALS9B \|PKHD1L1 \|SLC2A2 | 1.482072976 | 0.22864 | 1.09942 | 0.15114 | 0.00428 | 0.92642 |
| PAWR \|ABHD1 \|TNNC1 \|CYB5R2 \|ANLN \|TUBB4A \|BACE2 \|HYAL3 \|KANK1 \|SHF | 1.472020459 | 0.25586 | 0.89416 | 0.19503 | 0.39834 | 0.91833 |
| C8orf58 \|MPO \|DMKN \|LRRC1 \|LRRC58 \|CPEB3 \|LRRC14 \|ESR1 \|RPUSD2 \|TFAP2E | 1.464138106 | 0.22726 | 1.00953 | 0.11049 | 0.62297 | 0.91401 |
| TMEM170B \|LOXL3 \|MMRN1 \|KRBA2 \|DTX3 \|HDC \|EDAR \|CA8 \|NCKAP1 \|SLC41A2 | 1.456102613 | 0.18833 | 1.19685 | −0.0043 | −0.9029 | 0.93382 |
| CAPN5 \|SLC35F2 \|AS3MT \|PRELID2 \|IFNK \|KCNH3 \|TMEM242 \|EME1 \|NME3 \|DUSP18 | 1.444283743 | 0.25149 | 0.92081 | 0.11475 | 0.02306 | 0.92377 |
| TMED6 \|AGBL3 \|OAF \|PPM1E \|CDHR5 \|DTWD2 \|TBC1D30 \|PRRT3 \|CCDC34 \|SMPD3 | 1.436318555 | 0.21364 | 1.15179 | 0.0212 | 1.48548 | 0.92707 |
| SAC3D1 \|RAPH1 \|GTSF1 \|ADORA3 \|MPZL2 \|PRDM8 \|SPAG1 \|C16orf45 \|CCNB3 \|SPOCK1 | 1.427896614 | 0.23044 | 1.15396 | 0.03419 | −0.6404 | 0.93607 |
| PRSS3 \|EYS \|FUT2 \|ID3 \|C19orf48 \|C3orf18 \|ALAS2 \|ATXN7L2 \|TIGD2 \|SPAG4 | 1.421015555 | 0.24771 | 1.2028 | 0.00309 | −0.647 | 0.9289 |
| UTS2 \|C17orf97 \|SUV39H1 \|WDR18 \|IRAK2 \|CDK18 \|XYD2 \|FAM161B \|LYSMD1 \|LYSMD4 | 1.411700528 | 0.26271 | 0.91531 | 0.14926 | 2.43961 | 0.91987 |
| PCP2 \|TMEM101 \|GAL3ST4 \|VKORC1L1 \|ADD2 \|COQ3 \|CHN1 \|RLN1 \|PDGFRB \|AKAP5 | 1.405505203 | 0.2019 | 1.06214 | −0.0239 | 0.16555 | 0.92496 |
| PSAT1 \|RBM44 \|DCUN1D3 \|CORO6 \|SCOC \|CAPS2 \|C5orf34 \|FAM220A \|DNAJC6 \|LY6G5C | 1.397743159 | 0.25199 | 0.83554 | 0.15097 | −0.7604 | 0.93321 |
| RFESD \|PODXL \|PKP2 \|SMOC1 \|BMP1 \|GIMAP1 \|ESCO2 \|ZFPM2 \|GNG8 \|LNX2 | 1.384738752 | 0.24637 | 0.97416 | 0.03533 | 0.68686 | 0.92127 |
| SHBG \|ZNF416 \|FKBP7 \|LIPH \|POLN \|LYL1 \|C11orf63 \|PSD \|NDFIP2 \|GPR35 | 1.36954156 | 0.24994 | 1.14946 | 0.04548 | −0.5908 | 0.92877 |
| C11orf68 \|ZNF576 \|ZNF582 \|MRPL36 \|FAM19A2 \|TFR2 \|AMPH \|HOXA10 \|MOB4 \|IL5RA | 1.356966398 | 0.24731 | 1.00712 | 0.15689 | 0.95876 | 0.92573 |
| ATP8B3 \|ARG2 \|TTC28 \|ORAI3 \|SMTN \|MEGF11 \|GDF11 \|PNPLA7 \|CCIN \|C3orf14 | 1.34377386 | 0.25265 | 0.81212 | 0.15933 | 0.48865 | 0.91221 |
| CYP21A2 \|UPK3A \|EID3 \|UBE2T \|RBMY1J \|TSGA10 \|RSPH4A \|KIAA0040 \|NPDC1 \|CCDC153 | 1.333716239 | 0.25538 | 0.95215 | 0.10714 | −0.6196 | 0.92618 |
| ABHD14B \|H2AFY2 \|SLC25A27 \|SLC22A1 \|TUFT1 \|FAM186B \|SLCLA3 \|PLXNA2 \|FAM177B \|NINL | 1.323304562 | 0.21604 | 1.10578 | 0.00165 | −0.2066 | 0.93339 |
| RGL1 \|SNAPC1 \|SLC5A3 \|ZNF772 \|CTAGE9 \|FAM161A \|DENB59 \|CDKN1C \|ITGAD \|CEP112 | 1.314994774 | 0.26414 | 1.03551 | 0.09753 | −0.8348 | 0.93161 |
| C1orf189 \|EHD3 \|ZACN \|PLK4 \|DZANK1 \|CD1E \|IL23A \|PRCD \|FZD6 \|GLRX2 | 1.306232416 | 0.22488 | 1.09125 | 0.04194 | −0.9352 | 0.92758 |
| LCN10 \|KIF2C \|KIAA0825 \|KIF5C \|FPGT-INNI3K \|SIK1 \|FLT4 \|TXNDC2 \|CDON \|UNC5CL | 1.292433742 | 0.22126 | 1.00397 | 0.12515 | 0.78689 | 0.92083 |
| CDKN2A \|MAMDC4 \|NUMBL \|SPTSSB \|SLC10A1 \|CEL \|KIFC3 \|CLEC18A \|DNAH17 \|HYI | 1.281718598 | 0.18663 | 1.04936 | 0.02861 | 0.33652 | 0.90296 |
| ZNF607 \|TCFL5 \|FAM184A \|CRIP2 \|KATNAL2 \|C1orf109 \|BHAT \|CABLES2 \|RSPH10B \|GRIK1 | 1.276708971 | 0.22846 | 0.92429 | 0.07941 | −0.4597 | 0.91731 |
| PLCD3 \|DYNLL2 \|C1orf220 \|ZWINT \|RCOR2 \|MIS18A \|NXPE1 \|LYNX1 \|NUDT7 \|DNAH10 | 1.269784595 | 0.24551 | 0.92998 | 0.10997 | 1.08182 | 0.91843 |
| PIGW \|EPB41L1 \|SPAG16 \|ZNF630 \|CCDC28B \|ANGPTL3 \|ITGA1 \|TEX9 \|FEZ1 \|RNLS | 1.262555945 | 0.22787 | 1.11339 | −0.031 | −0.7492 | 0.93345 |
| SLC35G6 \|CEBPD \|HBD \|NUGGC \|SPATA24 \|ZSCAN5A \|DLL1 \|SLC16A10 \|HPR \|FBXO30 | 1.25257008 | 0.2346 | 1.1368 | 0.03621 | 2.08356 | 0.93321 |
| INSL3 \|DHRS9 \|CLECL1 \|COMTD1 \|PUSL1 \|YOD1 \|ZNF614 \|ENDOD1 \|QPCTL \|S100A13 | 1.240692911 | 0.25776 | 0.90484 | 0.16712 | −0.983 | 0.93616 |
| USP44 \|SLC25A35 \|FAM209A \|ANKRD30A \|ANKRD18A \|SLC12A3 \|ARMCX4 \|C1orf122 \|DCBLD2 \|DEPTOR | 1.233352012 | 0.21742 | 1.12094 | −0.0033 | −0.7856 | 0.92694 |
| ITGA2 \|ENPP5 \|PHLDB1 \|ZNF449 \|IFT81 \|C20orf196 \|LCMT2 \|RDH12 \|TRIP6 \|RPH3AL | 1.224024962 | 0.2404 | 1.10755 | 0.05771 | −0.368 | 0.92689 |
| MUL1 \|FAM89B \|ARHGAP6 \|FAM71F2 \|CHRFAM7A \|EBF4 \|SLC25A4 \|PIGM \|MORN2 \|CCDC121 | 1.218594279 | 0.25222 | 0.99496 | 0.0589 | −0.3617 | 0.92581 |
| ZNFS47 \|TOB2 \|KCNJ2 \|C3orf35 \|ZNF202 \|SPINK5 \|CXCR1 \|ROM1 \|MAGED4B \|ARHGAP8 | 1.209824259 | 0.25014 | 0.9973 | 0.07183 | −0.5193 | 0.92623 |
| GMEB2 \|MAGEF1 \|AMIGO2 \|DMD \|CDRT15 \|CALCRL \|SLC30A1 \|C9orf24 \|FAM8A1 \|TNFRSF8 | 1.198470674 | 0.22427 | 1.28065 | −0.0024 | 0.77548 | 0.92478 |
| HPD \|SCARF1 \|RTP4 \|C1QTNF7 \|TOMM34 \|TAS2R5 \|GPR157 \|ARHGAP22 \|ATP6V1G2 \|ZNRF1 | 1.190213346 | 0.22197 | 0.97094 | 0.08282 | 1.45601 | 0.92223 |
| DDX43 \|CHRNE \|CDKN3 \|XKR6 \|ZBTB39 \|P2RY11 \|PPP1R14A \|ZNF354C \|CHIRNA7 \|MUC5B | 1.181261481 | 0.22039 | 0.82895 | 0.11285 | −0.2534 | 0.91079 |
| PRLR \|ZNF436 \|CR1L \|TMEM140 \|RIN1 \|LIPT2 \|CHL1 \|FAM153C \|P4HA2 \|SYTL4 | 1.172313382 | 0.23731 | 1.29671 | −0.0045 | −0.0815 | 0.92251 |
| C3orf67 \|TRIM56 \|CDC42EP2 \|LRP3 \|KLKB1 \|ZNRF2 \|ZNF74 \|ZBTB45 \|B4GALNT4 \|SLC2A13 | 1.16072928 | 0.2474 | 0.97164 | 0.03434 | 0.76053 | 0.91021 |
| SPTSSA \|ZBTB3 \|LY6G6E \|NUFP1 \|LRRC4 \|FAM131A \|FDX1 \|TAS2R3 \|KIFC1 \|CLEC6A | 1.148488485 | 0.24119 | 0.89232 | 0.13953 | −0.4122 | 0.93439 |
| KCNC4 \|MAGED4 \|ZBTB6 \|ICMT \|KIF9 \|TRAIP \|KEL \|CNTNAP3 \|LY75 \|RUFY4 | 1.136201585 | 0.26119 | 0.93907 | 0.05237 | −0.7614 | 0.91942 |
| NDUFB6 \|IL17RE \|LRP12 \|FDXR \|BRSK1 \|KCTD21 \|PBLD \|PIM3 \|HOMEZ \|DNAH3 | 1.125277079 | 0.27559 | 0.87045 | 0.15426 | −0.562 | 0.91041 |
| BBS10 \|C9orf16 \|SYCE2 \|RNF122 \|GAS2L3 \|TSPYL4 \|TSPAN15 \|KCTD6 \|TRPV6 \|PSRC1 | 1.117440277 | 0.24534 | 0.91279 | 0.16966 | −0.314 | 0.933 |
| DTX1 \|CRABP2 \|GNAI3 \|LTK \|CNTF \|RRS1 \|PPM1H \|SPIN3 \|CACHD1 \|LDLRAD3 | 1.108809702 | 0.24204 | 0.94802 | 0.07241 | −0.6241 | 0.92492 |
| HYDIN \|QRICH2 \|SEMA7A \|ZMYND15 \|EVC \|KCNMB1 \|S1PR2 \|MSC \|PLEKHG6 \|MEGF8 | 1.100972406 | 0.22443 | 1.14629 | 0.00463 | 2.15175 | 0.91323 |
| ZNF219 \|ZNF385D \|TNKS1BP1 \|PLEKHA7 \|VIL1 \|RRH \|KIAA0895L \|ORC1 \|TRPA1 \|FADD | 1.092774963 | 0.22686 | 1.04821 | 0.08418 | −0.3401 | 0.92937 |
| CSRP2 \|COL24A1 \|MMACHC \|PHOSPHO2 \|FDXACB1 \|WDR63 \|FGF23 \|MAP2 \|AANAT \|DNAH11 | 1.085730671 | 0.22131 | 1.0714 | 0.10051 | −0.9157 | 0.9369 |
| ZBTB26 \|ZNF711 \|FOXP2 \|C18orf54 \|RPP40 \|NUDT19 \|FKBP14 \|FAM27C \|C12orf49 \|ZNF665 | 1.077078574 | 0.26141 | 0.90481 | 0.08557 | −0.4921 | 0.94055 |
| CCDC38 \|SCAND1 \|HIGD1C \|GAGE12H \|MRPS17 \|ASTL \|MATN2 \|TSPAN16 \|ZC2HC1C \|CIT | 1.065506111 | 0.21009 | 1.12287 | −0.0142 | −0.6166 | 0.93282 |
| CNGB1 \|MASP2 \|TSHB \|MCAT \|NAT6 \|CHRND \|MREG \|MMP24 \|NAIF1 \|DNAJB13 | 1.054175167 | 0.20612 | 0.92127 | 0.07135 | −0.1216 | 0.91622 |
| LYG2 \|ADAM12 \|OVCH1 \|NCAPG \|TMEM237 \|MCC \|CSF1 \|GPR75-ASB3 \|RYR2 \|DEFB4A | 1.043044959 | 0.18282 | 1.07456 | 0.0583 | −0.2517 | 0.93196 |
| ZG16B \|GAS2L1 \|CHGA \|FGFRL1 \|ANKS6 \|TBXA2R \|RBBP9 \|TRIM46 \|CYP4F3 \|TCTEX1D2 | 1.033685546 | 0.25065 | 0.99074 | 0.12124 | 2.92561 | 0.89802 |
| FBXL2 \|LGR4 \|SCNN1A \|MAPRE3 \|RRM2 \|UBTD2 \|TRIM6 \|SDR42E1 \|SPTBN5 \|SPA17 | 1.025338729 | 0.23223 | 1.02063 | 0.02934 | 1.15116 | 0.91428 |
| GRTP1 \|GYPB \|GRIN1 \|FBXO41 \|HCAR3 \|NOG \|PSD3 \|FLT1 \|FILIP1L \|RAP2B | 1.011487414 | 0.22817 | 0.9863 | 0.07159 | −0.888 | 0.91809 |
| OCLM \|LAGE3 \|PM20D1 \|HSPA1L \|FAM110A \|PSMG3 \|ASB2 \|SLC4A1 \|CACNB4 \|NODAL | 1.003833543 | 0.20448 | 1.17061 | 0.00775 | −0.5695 | 0.92611 |
| AGMAT \|GPC2 \|LIX1 \|LDB2 \|NLRP6 \|PP2D1 \|SGCA \|LRRC43 \|TTC22 \|ITGA7 | 0.99380556 | 0.21255 | 1.10782 | 0.06544 | −0.3471 | 0.91884 |
| CRIP3 \|ABHD14A-ACY1 \|C9orf84 \|ZNF300 \|WDR38 \|DNLZ \|TMEM204 \|PEAR1 \|TSSC4 \|SGSM1 | 0.98206269 | 0.23916 | 1.0376 | 0.05885 | 2.59253 | 0.90425 |
| WNT7A \|NDRG4 \|DNAH7 \|MON1A \|SLC45A4 \|RNFT2 \|PDE2A \|STX1B \|SHROOM1 \|TYMS | 0.969322682 | 0.22919 | 0.99971 | 0.01938 | 0.17993 | 0.89929 |
| LRRC36 \|TTYH1 \|VIN \|SERPINE2 \|MDGA1 \|PANK1 \|IL10 \|TOP2A \|TSPY8 \|CISH | 0.958428058 | 0.22659 | 0.94921 | 0.00724 | 0.45178 | 0.90623 |
| C1R \|RHOXF2 \|RHOBTB1 \|DMBT1 \|CDCA7 \|PROC \|CCDC13 \|MAGEE1 \|SEMA4G \|SERPINB2 | 0.946730467 | 0.19473 | 1.11621 | 0.03454 | −0.6893 | 0.92292 |
| THBS4 \|GFOD1 \|CHAF1B \|FBXL18 \|FBXO5 \|SLC26A1 \|RHEBL1 \|POTEG \|MGAT3 \|INTS5 | 0.933238775 | 0.23731 | 0.79047 | 0.17256 | −0.4378 | 0.91591 |
| PRRG1 \|CHAC2 \|OR10AD1 \|SLC22A18AS \|FBXL13 \|CENPB \|ZNF524 \|HIGD1A \|STK33 \|VMO1 | 0.918557211 | 0.23007 | 1.02915 | 0.04239 | −0.6259 | 0.93531 |
| ADAMTS13 \|FBXL14 \|COL9A1 \|PRAF2 \|KCNJ1 \|HMHB1 \|TMIE \|BTBD18 \|GPR21 \|RBP2 | 0.911021103 | 0.18337 | 1.19964 | 0.02257 | 0.2069 | 0.92506 |
| REM2 \|TRPM4 \|RNF39 \|CARD17 \|SLC22A16 \|ZNF705A \|PLOD2 \|GOLGA6D \|C10orf35 \|STOML1 | 0.90345813 | 0.23183 | 0.88239 | 0.18791 | −0.3024 | 0.91824 |
| GADD45A \|CCDC150 \|RGS20 \|CARNS1 \|PGC \|PRKG2 \|FBXO10 \|SH3TC2 \|DNM1 \|PCDH9 | 0.893803743 | 0.20953 | 1.01788 | 0.05235 | −0.4363 | 0.9282 |
| TUB \|CYP24A1 \|ZNF70 \|CLDN24 \|GSTA1 \|GRIN2C \|POU5F1 \|RLN2 \|LTB4R2 \|ZNF287 | 0.884581185 | 0.21804 | 1.08047 | 0.03625 | 0.36255 | 0.91893 |
| WDR5B \|GRHL2 \|TTC39A \|ZBTB9 \|PDP2 \|SOCS3 \|PET117 \|GRHL1 \|RUNX1T1 \|PNMA1 | 0.876804293 | 0.24949 | 0.95558 | 0.08385 | 0.16875 | 0.91853 |
| STYK1 \|CBX8 \|SLITRK4 \|ZNF77 \|CCDC120 \|SH3BP4 \|ETV5 \|LINGO3 \|SEZ6 \|NME9 | 0.86969009 | 0.2442 | 1.21813 | 0.03808 | 1.49257 | 0.92142 |
| NAGS \|RAB40AL \|TMEM45B \|CENPF \|USP31 \|TAS2R20 \|ALDH4A1 \|FKBPL \|TMEM177 \|FKBP6 | 0.859702572 | 0.22343 | 1.00312 | 0.01724 | −0.1039 | 0.93305 |
| CLSPN \|PRSS21 \|PELO \|MPLKIP \|ABHD15 \|EHD4 \|C19orf44 \|P2RY6 \|LPA \|NACC2 | 0.85187184 | 0.24835 | 1.0597 | 0.09749 | −0.0974 | 0.9201 |
| ZMYND10 \|RSPH10B2 \|CLMP \|CHST10 \|CCDC154 \|MYZAP \|B3GNT7 \|PEX11G \|ALG14 \|DKK3 | 0.844025463 | 0.21684 | 0.98329 | 0.11037 | −0.391 | 0.90815 |
| RAD54B \|EPHA2 \|SOX5 \|PKN3 \|TMEM60 \|PDZD3 \|TIFA \|DENND2C \|CYP4F22 \|MIA | 0.83508499 | 0.23594 | 0.95276 | 0.11607 | 0.01545 | 0.92264 |
| PWWP2B \|FEZF2 \|DACT1 \|THBD \|TMTC1 \|NEK11 \|RSG1 \|STEAP3 \|OR2W3 \|AURKB | 0.825621523 | 0.2465 | 0.89108 | 0.06943 | −0.9773 | 0.91329 |
| EPHB1 \|KIF18A \|BMP6 \|CLEC18C \|LGALS4 \|DNAJC25 \|MYCBPAP \|SLC9A5 \|PTK7 \|FBXW9 | 0.815877316 | 0.23411 | 0.99729 | 0.09008 | 0.12494 | 0.91096 |
| RAPSN \|SEZ6L \|PRTFDC1 \|ZNF415 \|ARMC5 \|BEGAIN \|WNT10A \|C18orf32 \|MAP1A \|EREG | 0.8071901 | 0.21336 | 0.97245 | 0.06888 | −0.4206 | 0.90704 |
| DUSP3 \|GTF2H5 \|UNC5A \|ZNF80 \|PLEKHGS \|C21orf2 \|B3GAT1 \|PSPH \|GAS2 \|BUB1 | 0.796823105 | 0.21371 | 0.96784 | 0.11623 | −0.6842 | 0.91506 |
| GFPT2 \|ZNF404 \|SLC5A2 \|OIP5 \|RARB \|CLEC2L \|C1orf56 \|EDA \|IL5 \|LYVE1 | 0.787854073 | 0.18591 | 1.02895 | 0.05154 | −0.5518 | 0.93429 |
| MS4A2 \|TRIM36 \|MOCS1 \|GPR15 \|ZNF727 \|AKAP6 \|MYO7B \|NEGR1 \|KBTBD6 \|B3GNT5 | 0.781043013 | 0.20007 | 1.10408 | 0.03917 | 2.56381 | 0.93637 |
| CERCAM \|MEGF6 \|PYGO2 \|ANKRD16 \|TNFRSF21 \|NPHP1 \|EMP1 \|CPS1 \|PKD2L2 \|GLI3 | 0.77147076 | 0.22151 | 0.90738 | 0.04278 | 0.02161 | 0.92453 |
| TMEM53 \|RAB39A \|BAIAP2L1 \|MPPED2 \|C15orf62 \|PMFBP1 \|TPRN \|TRAM2 \|ITGA9 \|PRX | 0.764232875 | 0.22519 | 0.96001 | 0.02988 | −0.2319 | 0.92049 |
| TREML4 \|PAQR6 \|KLHDC8B \|CREG1 \|KIAA1958 \|DNMT3B \|PCGF6 \|LAMA5 \|GPR160 \|TMEM88 | 0.75522751 | 0.26151 | 0.81537 | 0.15693 | −0.0317 | 0.90032 |
| AQP10 \|COL9A3 \|OR1L4 \|MYH7B \|LYG1 \|RAMP2 \|ZNF578 \|ADAM32 \|PLXNB1 \|SLC25A10 | 0.74534934 | 0.20582 | 0.97354 | 0.06537 | 0.50107 | 0.92169 |
| SCARF2 \|DDR2 \|CYP2E1 \|RTN4R \|TNFRSF9 \|ARMC9 \|LIPC \|MAGI3 \|PM20D2 \|CIB3 | 0.732817147 | 0.20245 | 1.15404 | 0.00643 | −0.6911 | 0.92633 |
| DNAH6 \|TNNT2 \|CRYM \|IKZF4 \|CDKN2AIPNL \|STON1 \|ZKSCAN2 \|FAM25C \|URB2 \|RNF212 | 0.725875566 | 0.21525 | 1.15962 | 0.0166 | 1.15608 | 0.93121 |
| NDC80 \|RECQL4 \|ZNF829 \|WNT4 \|COL5A3 \|SPRED2 \|SLC2A8 \|RAVER2 \|DCLK2 \|PXMP2 | 0.719536981 | 0.26114 | 0.94082 | 0.08589 | −0.4934 | 0.89778 |
| S1PR3 \|EFNA4 \|CCDC85C \|FSCN3 \|PGBD1 \|TOMM20L \|KLF9 \|DIXDC1 \|KANK3 \|FAM189B | 0.711003974 | 0.22712 | 0.93005 | 0.06715 | −0.0387 | 0.92294 |
| PLAG1 \|FAM212A \|MC1R \|PTPDC1 \|HECW2 \|CLIP3 \|SLC25A23 \|COL3A1 \|S100A1 \|CDC20 | 0.704393725 | 0.22504 | 1.07342 | 0.00261 | 0.34525 | 0.91931 |
| CYP4F12 \|GPR55 \|PTCRA \|ZNF662 \|PTGIR \|BPBL \|CLEC18B \|UAP1L1 \|ATXN7L3B \|ANKRD308 | 0.697742628 | 0.19523 | 1.06676 | 0.09951 | 0.81477 | 0.9099 |
| MARCKS \|KIAA0895 \|PPP1R1B \|UBE2C \|WDR90 \|NOXA1 \|ATP6V1B1 \|ATP6V1C2 \|VMAC \|FAMI66B | 0.691413278 | 0.23512 | 0.85391 | 0.09103 | 0.16725 | 0.90865 |
| CCDC152 \|ZNF275 \|TMEM132A \|FPR3 \|FRMD5 \|ARMC3 \|ANKRD20A2 \|DTL \|MOCOS \|UNC13B | 0.683196538 | 0.21508 | 1.09505 | 0.03727 | −0.3664 | 0.92887 |
| LRRC39 \|IFI27L1 \|TET1 \|PCDHB5 \|ZNF517 \|EFNB1 \|VWF \|POPDC2 \|MYO7A \|KIAA1257 | 0.670058919 | 0.22301 | 1.12061 | 0.06493 | −0.8329 | 0.92866 |
| GJB7 \|ZC3H12C \|PCGF2 \|MFAP4 \|NOS3 \|SERPINC1 \|RBMY1E \|P2RY2 \|PROS1 \|SFR1 | 0.653986432 | 0.22966 | 1.13254 | 0.03029 | −0.3688 | 0.92095 |
| TTC36 \|CDR1 \|TPH1 \|SARDH \|NTNG2 \|DECR2 \|ALDH8A1 \|CENPL \|TCF19 \|BEX5 | 0.640202302 | 0.2106 | 1.28549 | 0.01323 | 1.56733 | 0.93193 |
| ACAN \|RFTN2 \|HTR3A \|LRRC29 \|ZNF628 \|JSRP1 \|SPATA3 \|ACOT1 \|IGFBP3 \|CDC42BPG | 0.633798274 | 0.18769 | 1.09239 | 0.0632 | 0.58713 | 0.91636 |
| MCM2 \|KTI12 \|NUDT3 \|CATSPER3 \|PARM1 \|KCNAB1 \|TCN1 \|BBS5 \|ABCA6 \|WNT10B | 0.624759891 | 0.21759 | 1.03477 | 0.04294 | −0.5099 | 0.93912 |
| TMSB15B \|NUDT6 \|PCSK1 \|CABYR \|ANGPTL6 \|SH3D21 \|RBMS3 \|F2RL1 \|ALDH1A2 \|CCT68 | 0.616971868 | 0.23465 | 1.16822 | 0.05689 | −0.4279 | 0.93176 |
| RAB20 \|ZNF747 \|MASP1 \|LNX1 \|AATK \|MTTP \|TMEM81 \|KIRREL3 \|HARBI1 \|KIF19 | 0.611738276 | 0.2203 | 0.94072 | 0.09519 | 1.17154 | 0.92564 |
| SCT \|LAYN \|RAB33B \|MUC7 \|TP63 \|C6orf10 \|SNTN \|RBFOX2 \|LTBP2 \|TAS2R41 | 0.604673409 | 0.19519 | 1.12832 | −0.0038 | −0.2802 | 0.93455 |
| DGCR6 \|TTC30B \|RBKS \|TNXB \|MRPS12 \|FAM20C \|METTL18 \|SNTA1 \|PCDH15 \|TTC26 | 0.595138138 | 0.26922 | 0.9639 | 0.10756 | 1.26727 | 0.93274 |
| C16orf86 \|CCDC51 \|PMEPA1 \|CXCL10 \|RAB44 \|CBX2 \|TEX30 \|TMEM187 \|C7orf61 \|MPZ | 0.587064914 | 0.23924 | 0.95474 | −0.0092 | −0.458 | 0.91416 |
| SLC27A5 \|RAB23 \|PCLO \|ST7-OT4 \|HCAR2 \|JPH4 \|CEBPG \|ENPP2 \|DMRTC1B \|RANBP17 | 0.577978902 | 0.22068 | 1.10692 | 0.03254 | −0.1103 | 0.9248 |
| CTTNBP2 \|OPA3 \|VLDLR \|CBR3 \|TIMM8A \|LACC1 \|PIGZ \|FAM131B \|PAX6 \|PARD6B | 0.566232357 | 0.26317 | 0.86061 | 0.10294 | −0.229 | 0.91219 |
| NUP210L \|SLC48A1 \|GPSM1 \|HES4 \|RHBDL2 \|OSGIN1 \|ZNF280B \|CLIC5 \|B3GALNT1 \|CELF6 | 0.555640915 | 0.22782 | 1.01751 | 0.03676 | −0.1929 | 0.92412 |
| HNRNPA0 \|GNAI1 \|SDSL \|OAZ3 \|GABRR2 \|APOLD1 \|HCFC1R1 \|TRIM69 \|SIGLEC12 \|AKT1S1 | 0.548567434 | 0.26281 | 0.94205 | 0.14751 | −0.6939 | 0.92595 |
| CATSPERB \|MAPT \|TGFB1I1 \|SERPINB10 \|CABP5 \|ANKRD13B \|CPNE9 \|HKDC1 \|APOL4 \|COL4A4 | 0.5369546 | 0.19954 | 1.01128 | 0.07216 | 2.171.75 | 0.92215 |
| TACR1 \|C15orf52 \|MTRNR2L8 \|ARMC12 \|PPP2R3A \|C3orf49 \|SIGLEC11 \|FAM86B2 \|CENPH \|TP53INP2 | 0.530569563 | 0.22833 | 1.11614 | 0.08628 | −0.5229 | 0.92839 |
| EPGN \|CRB3 \|CUX2 \|SLC18A2 \|NUP62CL \|TTK \|KCTD1 \|MAD2L1 \|CIB2 \|GCNT4 | 0.520546661 | 0.22427 | 0.93876 | 0.03073 | −0.2513 | 0.92874 |
| GOLGA8G \|KLHL15 \|HSD11B1 \|OR2B11 \|PRDM5 \|PDCD1 \|CD80 \|TBC1D29 \|KCNK6 \|SNAI3 | 0.51320824 | 0.20433 | 1.21505 | 0.05322 | 0.68777 | 0.91929 |
| CHRNB2 \|DMRTC1 \|EVC2 \|TMEM56-RWDD3 \|OTUB2 \|KLHL32 \|SLC39A14 \|LHX4 \|FAM57A \|DCHS1 | 0.505704698 | 0.24271 | 0.85997 | 0.09232 | −0.319 | 0.91208 |
| EFNA5 \|GPT2 \|ZNF502 \|PAQR7 \|TP53TG3 \|PDE3A \|RASAL2 \|UCP3 \|DLC1 \|PPARG | 0.498797128 | 0.22057 | 1.14118 | 0.06311 | −0.4788 | 0.93401 |
| GNRHR \|SSC5D \|CLEC4G \|ZNF239 \|ANO5 \|ZNF213 \|NLGN2 \|RTN4RL2 \|LPL \|EGLN3 | 0.487502456 | 0.18876 | 1.08551 | 0.00774 | 4.04789 | 0.92031 |
| CCR8 \|NMNAT3 \|C10orf10 \|WDR88 \|ACER2 \|FAM200A \|SORCS3 \|SYT1 \|ARL13A \|NAV2 | 0.475549354 | 0.20364 | 1.13666 | 0.07465 | −0.2359 | 0.91762 |
| GRK4 \|SPATA21 \|ABLIM2 \|ACOX2 \|CCNE1 \|FAM209B \|MAMLD1 \|CNN3 \|SPIRE2 \|TPTE | 0.465206603 | 0.2214 | 0.89551 | 0.04527 | −0.1098 | 0.90676 |
| ZDHHC21 \|GRB14 \|ETV4 \|YDJC \|ACVRL1 \|SLAMF8 \|TMED8 \|TMEM201 \|HTRA4 \|THNSL1 | 0.455052132 | 0.25101 | 0.99911 | 0.04714 | −0.5826 | 0.92154 |
| NEK10 \|OGN \|UBAP1L \|PEG10 \|PURB \|MAP3K9 \|ROR2 \|SNN \|ZNF610 \|ARL3 | 0.4442439 | 0.23219 | 0.82263 | 0.10098 | −0.257 | 0.93302 |
| TREM2 \|ANK2 \|YPEL4 \|SRPK3 \|BEND6 \|IQCH \|IGSF22 \|NID2 \|ZDBF2 \|KRT6A | 0.432323026 | 0.18207 | 1.00483 | 0.05713 | −0.4202 | 0.9213 |
| S100A5 \|PNMA3 \|DAZL \|COL2A1 \|ARHGEF4 \|GATC \|HSF5 \|DPEP3 \|ZNF788 \|FAM72D | 0.422951289 | 0.20714 | 0.95789 | 0.13844 | 2.43347 | 0.91119 |
| HIST1H3G \|SHISA4 \|ADSSL1 \|FXYD1 \|BATF3 \|SLC1A1 \|DNAAF1 \|IFI27 \|LRP5 \|RUNDC3B | 0.415359596 | 0.22729 | 0.89203 | 0.08528 | 1.13932 | 0.90588 |
| GFRA2 \|IFITM5 \|BIVM \|NOTCH4 \|ZNF554 \|ZNF233 \|CRYBB1 \|SLC6A20 \|EXO1 \|FSCN2 | 0.404993613 | 0.20783 | 1.12768 | 0.04081 | 1.7095 | 0.91763 |
| N6AMT1 \|MYO10 \|ESR2 \|GATA1 \|LRRC20 \|FITM1 \|PDE7B \|C14orf28 \|TMEM186 \|TMEM102 | 0.395206258 | 0.24949 | 0.87833 | 0.04345 | −0.5904 | 0.92797 |
| HOXB4 \|TMSB4Y \|TRO \|KLHDC9 \|PCYT1B \|THAP8 \|ACVR1C \|C21orf38 \|BEND4 \|UNC119B | 0.382908497 | 0.24742 | 1.00612 | 0.06383 | −0.3265 | 0.92827 |
| IFNG \|CDC45 \|NT5M \|SLC44A5 \|ITIH1 \|ARL5B \|TCTE3 \|ADGB \|TOM1L1 \|DAGLA | 0.371094514 | 0.22997 | 1.0544 | 0.09187 | 0.1896 | 0.93805 |
| CDNF \|CYP19A1 \|SLC9A3R2 \|IL20RB \|C15orf54 \|ILDR1 \|SSPN \|FRAT1 \|AK4 \|FTCD | 0.363305972 | 0.21214 | 1.07033 | 0.07632 | 0.97887 | 0.92377 |
| DLK2 \|NAP1L3 \|F8 \|CNGA1 \|COL8A2 \|OR52I1 \|RIMBP3B \|ECT2L \|GLS2 \|GPAT2 | 0.356830912 | 0.18502 | 1.08068 | 0.05453 | 1.78017 | 0.93632 |
| EHHADH \|MAP7 \|TMEM86B \|ZNF787 \|ZNF696 \|PNPLA1 \|SLC5A9 \|KCNG1 \|CEP19 \|COL21A1 | 0.341931357 | 0.19447 | 1.04661 | 0.0711 | −0.7074 | 0.91456 |
| WNT16 \|TTC9 \|CCDC122 \|MAP7D2 \|FGFR2 \|SENP8 \|MRPL41 \|PRRG4 \|WDR34 \|TMCC2 | 0.325451711 | 0.23652 | 0.99122 | 0.00592 | −0.8632 | 0.92494 |
| LEKR1 \|ULBP3 \|ARMCX3 \|KLHL10 \|NACAD \|G0S2 \|KRT23 \|COL11A2 \|CDK20 \|TMEM203 | 0.316387127 | 0.23205 | 1.03867 | 0.01749 | −0.4982 | 0.92202 |
| SLC2A5 \|BFSP1 \|GPR180 \|PPM1J \|DNAI2 \|CORO2A \|MAGI1 \|ANXA9 \|SDS \|MAFG | 0.306696281 | 0.20882 | 1.2112 | 0.00925 | 0.63913 | 0.92016 |
| FAM174A \|KRT9 \|GNG11 \|ZNF597 \|C17orf67 \|STK31 \|HECTD2 \|UGT8 \|FBXL12 \|TXNRD3 | 0.298526378 | 0.22608 | 0.98935 | 0.0929 | −0.6604 | 0.93647 |
| GLIPR1L1 \|FBLN7 \|ZNF462 \|FAM166A \|GLIPR1L2 \|NMB \|DACH1 \|TRPV4 \|PAGE2B \|ZMAT4 | 0.289401526 | 0.20338 | 1.09115 | 0.01934 | 0.79334 | 0.92921 |
| FBN1 \|SPATA2 \|SNTG2 \|TST \|CASC1 \|IRF2BP1 \|PODXL2 \|GJA9 \|ZBTB47 \|WASF3 | 0.279430484 | 0.24403 | 1.00457 | 0.06002 | −0.3711 | 0.91887 |
| SLC27A2 \|DDCDC2B \|RAPGEF3 \|ZNF775 \|FOLH1 \|TMEM86A \|MARS2 \|CMTM4 \|ZFP91 \|BHLA3 | 0.268019889 | 0.25619 | 0.82443 | 0.07941 | −0.6616 | 0.92084 |
| OR2T3 \|ETV7 \|SPAG17 \|RPL39L \|CHST2 \|PPP1R9A \|CHST7 \|TEAD2 \|DOCK1 \|TYSND1 | 0.259682527 | 0.21976 | 1.1513 | −0.0475 | −0.75 | 0.92083 |
| TRIM61 \|MSANTD3-TMEFF1 \|CERS3 \|CACNA2D1 \|ZSWIM3 \|COMP \|MYBPC1 \|DHRS11 \|CLNK \|EFCAB5 | 0.249427552 | 0.17578 | 1.17722 | 0.02933 | −0.2542 | 0.9353 |
| IRAK1BP1 \|STARD8 \|GINS2 \|LAMA2 \|SIGLEC15 \|PPP4R4 \|VENTX \|AMOTL1 \|ITGA10 \|ZNF608 | 0.240828991 | 0.22252 | 1.10703 | 0.08445 | 0.08488 | 0.92464 |
| PEX11A \|WDR31 \|IL4I1 \|ZNF629 \|SHE \|RPA4 \|CHAD \|SLC12A1 \|PRB4 \|KRT18 | 0.230078266 | 0.22087 | 1.10972 | 0.08123 | −0.5285 | 0.9223 |
| KIF11 \|ZFP37 \|HOXC4 \|TMEM184A \|PTGR1 \|POC1A \|SMARCA1 \|RET \|SIGLEC1 \|IYD | 0.219617172 | 0.20451 | 1.04435 | 0.01721 | 1.89398 | 0.91762 |
| CD1B \|TAS2R14 \|GPRC5D \|ATP7B \|FOXRED2 \|RAB25 \|GUCY2D \|ANGPTL7 \|NUDT18 \|LRRC34 | 0.20984966 | 0.21523 | 1.19643 | 0.05632 | −0.5343 | 0.93081 |
| MYO1H \|C1orf74 \|LTF \|TMEM129 \|GPR22 \|FAM111B \|ASF1B \|TMEM8B \|DOCK3 \|ROPN1B | 0.202267527 | 0.22721 | 0.92914 | 0.03514 | 0.2305 | 0.93052 |
| SEMA6C \|MARCKSL1 \|PDZD9 \|PDGFB \|FARP1 \|ARHGEF19 \|GPR3 \|SLC38A4 \|ELN \|SH3RF2 | 0.19282486 | 0.21107 | 1.13787 | −0.0109 | 1.24794 | 0.91128 |
| LAMB3 \|MANSC1 \|ZC3H12B \|NXPE4 \|HIC2 \|CCDC102A \|SLC5A11 \|PDGFRA \|BAALC \|SH3PXD2B | 0.185573368 | 0.2067 | 0.99592 | 0.05009 | −0.872 | 0.92513 |
| PIK3R3 \|ARNTL2 \|ZNF697 \|PCDH1 \|ALG1L \|TRIB3 \|C1orf198 \|TNFRSF18 \|CNGB3 \|TNFRSF4 | 0.177100919 | 0.22531 | 0.93459 | 0.03087 | −0.5409 | 0.91281 |
| CD163L1 \|CCNA2 \|ZSWIM4 \|GPD1 \|CAND2 \|MAP1LC3A \|PLG \|LMOD2 \|AFAP1L2 \|HIST1H1A | 0.166924557 | 0.20135 | 1.14783 | 0.02782 | −0.6579 | 0.92978 |
| ZNF285 \|DSC1 \|PAK3 \|NUDT10 \|CHAT \|TBX6 \|BHLHA15 \|ZNF492 \|C16orf74 \|HSD17B13 | 0.160977526 | 0.19684 | 1.15913 | −0.0287 | −0.5943 | 0.92332 |
| ZGLP1 \|SLC35G2 \|CCND1 \|STAP2 \|IQCD \|FSCN1 \|CA13 \|ECM1 \|ANKK1 \|FZD2 | 0.153017167 | 0.2249 | 1.09606 | 0.05211 | −0.6046 | 0.91627 |
| DISP2 \|CNTD1 \|SNPH \|GFOD2 \|DZIP1L \|PAPLN \|SGPP2 \|FAM213B \|SLC17A3 \|TGM2 | 0.141714501 | 0.24649 | 1.05512 | 0.08824 | −0.1802 | 0.91864 |
| TM4SF19 \|HINT3 \|TMPRSS6 \|VCX3B \|CXCR3 \|MYO3B \|CDKL3 \|SPTA1 \|MYCT1 \|MECOM | 0.129988927 | 0.19752 | 1.26111 | −0.0091 | −0.6153 | 0.93837 |
| EXTL2 \|MAPK10 \|TMPRSS3 \|ADM \|AKR1C2 \|FSTL3 \|PI4K2A \|HAMP \|PHF13 \|FBXL8 | 0.120338032 | 0.22507 | 0.96508 | 0.13645 | 0.49681 | 0.92118 |
| NCAPH \|TAS2R50 \|HEATR4 \|EID2 \|SLC19A2 \|GPC1 \|KCTD4 \|RASSF8 \|PPIL6 \|TSHZ3 | 0.112171242 | 0.22815 | 0.88478 | 0.1296 | −0.7508 | 0.93467 |
| WDR66 \|TEX22 \|HLA-G \|ANKRD22 \|CTNNA3 \|TDRD6 \|CCDC171 \|RPRML \|KLHL23 \|MEF2B | 0.099426764 | 0.23302 | 1.09982 | 0.03238 | −0.9315 | 0.93338 |
| PTGES3L-AARSD1 \|COL13A1 \|NRN1L \|CLEC11A \|ARVCF \|OR5K2 \|NUAK1 \|THEM6 \|COL7A1 \|PI16 | 0.086630538 | 0.20876 | 1.00033 | 0.05866 | 0.64597 | 0.90645 |
| C11orf94 \|APOC1 \|DNALI1 \|KCTD19 \|FAM210B \|GPR34 \|IQSEC3 \|LDOC1 \|TXLNB \|SHCBP1 | 0.073710709 | 0.2287 | 1.06028 | 0.0480S | −0.272 | 0.92503 |
| CXCL5 \|ZNF804A \|OR5B21 \|STARD13 \|DOC2A \|TNNI3 \|C14orf105 \|ZNF853 \|LIMCH1 \|GPM6A | 0.060950252 | 0.19064 | 1.05078 | 0.08098 | −0.1724 | 0.92299 |
| ASPM \|ASIC1 \|MLF1 \|SLC16A11 \|C2orf81 \|RAD9B \|E2F2 \|CYP2U1 \|NXF5 \|FBXL15 | 0.053334623 | 0.25642 | 1.02178 | −0.0184 | −0.3876 | 0.92141 |
| CDC6 \|PCBP3 \|GSTM3 \|FAM160A1 \|ENAM \|MYO5C \|ZNF521 \|GML \|CNTN4 \|GDAP1L1 | 0.046139273 | 0.20639 | 1.06644 | 0.04861 | −0.225 | 0.93349 |
| DCC \|ZBED4 \|IL7 \|B3GALT2 \|KBTBD3 \|SLC4A11 \|TF \|FBLN5 \|RGS7 \|ERVFRD-1 | 0.036762887 | 0.21093 | 0.98893 | 0.06455 | 1.05801 | 0.93412 |
| SP7 \|EFCAB10 \|REEP6 \|NUDT12 \|ARHGAP29 \|DNAI1 \|LRGUK \|RABIF \|KIAA0408 \|CITED1 | 0.028726172 | 0.23735 | 0.96261 | 0.12531 | −0.091 | 0.93562 |
| PYCR1 \|CRLF1 \|OR2A4 \|PPP1R17 \|SPDYE4 \|LIFR \|RPS6KL1 \|HJURP \|C22orf23 \|NAP1L2 | 0.019800411 | 0.19449 | 0.95402 | 0.07527 | −0.3434 | 0.92511 |
| HS3ST3B1 \|ZNF572 \|ABCA12 \|PPAN-P2RY11 \|THNSL2 \|LRRC7 \|KHDRBS2 \|PARD3B \|ULBP2 \|AQP7 | 0.012241905 | 0.20084 | 1.28237 | −0.0118 | 0.15431 | 0.9273 |
| CUZD1 \|IGSF10 \|ZNF491 \|CLEC9A \|AXDND1 \|CPLX2 \|SPINK4 \|FAM174B \|SYN3 \|CD3EAP | 0.004808308 | 0.20216 | 1.21871 | −0.0087 | −0.1418 | 0.9319 |
| TNFRSF17 \|BMP8A \|FGD1 \|SPRY1 \|CXorf58 \|C2orf48 \|ZNF296 \|KCNH8 \|TNFRSF13C \|SLC25A33 | −0.00386279 | 0.24156 | 0.98868 | 0.13144 | 0.80295 | 0.91987 |
| MYBL2 \|GOLGABF \|SLC9A3 \|HSPA2 \|SPATA25 \|SAG \|C1orf105 \|GSTO2 \|PKIB \|NKD1 | −0.00977584 | 0.2146 | 0.99817 | −0.0066 | 0.14573 | 0.92664 |
| ABHD8 \|ZNF177 \|PLEK2 \|SLC25A48 \|UGT2B17 \|SPRY2 \|C1QTNF5 \|FBXO39 \|TMPPE \|ESPN | −0.01609452 | 0.19709 | 1.07387 | 0.02391 | −0.6031 | 0.91167 |
| SPOCK3 \|TREML2 \|DUSP19 \|OLFML28 \|GRIK4 \|CCL27 \|MAP6D1 \|ISPD \|DRD4 \|GNA11 | −0.02635054 | 0.23218 | 1.00788 | 0.02925 | −0.2177 | 0.92112 |
| ALKBH4 \|OSM \|PDIA2 \|MOG \|GYPE \|FSD1 \|CTNNAL1 \|GATA2 \|KLHL14 \|LRP2 | −0.03409677 | 0.22082 | 0.96439 | 0.0289 | 0.22712 | 0.91161 |
| TNNI3K \|ZSWIM5 \|DERL3 \|CALR3 \|TNC \|PPP1R13L \|PFN4 \|DHRS4-AS1 \|ASB16 \|PLEKHH2 | −0.04382247 | 0.2354 | 0.94313 | 0.07322 | −0.3309 | 0.93176 |
| NOTCH3 \|ZNF660 \|C9orf131 \|TK1 \|PDGFA \|CD70 \|KCND3 \|PTH2 \|FAM47E-STBD1 \|C17orf53 | −0.05268219 | 0.22198 | 1.06203 | 0.04309 | −0.1991 | 0.91942 |
| ZNF329 \|CDC42EP1 \|NAP1L5 \|MFSD2B \|PGLYRP1 \|FAM171A1 \|PGM2L1 \|TUSC3 \|CGN \|P4HA3 | −0.06603391 | 0.23878 | 0.92059 | 0.13043 | 0.01375 | 0.92084 |
| FNDC5 \|FIZ1 \|SIRT4 \|ROBO1 \|ZNF575 \|FDCSP \|NEUROD2 \|CAPN8 \|CCDC40 \|PARD6A | −0.07832346 | 0.23534 | 0.99805 | 0.02378 | −0.0271 | 0.92507 |
| RGS5 \|TRIMS9 \|NR1I3 \|TACR2 \|AMELX \|EXPH5 \|CELSR2 \|MAOA \|ATG9B \|APOC2 | −0.08646948 | 0.18488 | 1.14876 | 0.01504 | 2.40764 | 0.92328 |
| SPANXB1 \|SPTLC3 \|F2 \|HOXA1 \|MYH14 \|PHLDA1 \|ZCCHC18 \|TRPM1 \|WDR93 \|SCGB3A1 | −0.09309631 | 0.19047 | 1.0948 | 0.02863 | −0.0885 | 0.91679 |
| KRTAP5-7 \|HTRA1 \|MBLAC2 \|NEURL2 \|SCUBE3 \|ZNF366 \|DSP \|CCR6 \|PPFIA3 \|SYP | −0.09738107 | 0.2256 | 1.05654 | 0.0916 | 1.2932 | 0.91934 |
| ENTPD3 \|LIPF \|SAA1 \|TECTA \|ABCA9 \|H1FNT \|SLC34A3 \|AHDC1 \|EPPK1 \|GALNT4 | −0.1039457 | 0.18106 | 1.14739 | 0.05565 | 3.02171 | 0.92799 |
| ETNK2 \|NRXN2 \|SLC1A7 \|SAA2 \|MKRN3 \|GHR \|CBX6 \|PRSS16 \|TRIM51 \|SBK1 | −0.11223475 | 0.19319 | 1.09437 | 0.03067 | 1.31635 | 0.91698 |
| TNFSF14 \|ACY3 \|PLA2G4B \|HCRTR1 \|TLN2 \|BTNL9 \|SERINC2 \|AKR7A3 \|KRT13 \|AIFM2 | −0.12169516 | 0.199 | 1.0937 | 0.04237 | 0.54963 | 0.9241 |
| EGFL8 \|SLC22A17 \|TTLL10 \|CLEC1A \|PLS3 \|HLF \|CHRM4 \|NAPSA \|PNOC \|ADCY6 | −0.13053059 | 0.16487 | 1.12059 | 0.00448 | −0.8424 | 0.92476 |
| SEMA3A \|VSIG10 \|TMPRSS5 \|CXCR5 \|TAL1 \|HPX \|AIRE \|PLEKHA4 \|SLC7A9 \|C1QC | −0.13669379 | 0.16687 | 1.131 | 0.01559 | 0.31388 | 0.91123 |
| ITIDH3 \|PDRG1 \|NCR3LG1 \|NMUR1 \|LRRC49 \|PTPRZ1 \|LRP11 \|PTGFRN \|VCX3A \|SLC26A4 | −0.14957054 | 0.22034 | 1.03832 | 0.05693 | −0.6797 | 0.91735 |
| RTKN2 \|ERRFI1 \|ADORA2B \|ARHGAP23 \|PRKG1 \|MCF2 \|BIK \|TMPRSS11D \|RDM1 \|ELF3 | −0.16138903 | 0.23768 | 0.95957 | 0.0529 | 2.23048 | 0.9252 |
| JAM2 \|LMTK3 \|FITM2 \|TP53TG5 \|FAM221B \|PODN \|PCDH12 \|AMH \|ALOX15B \|PLXNB3 | −0.17078837 | 0.21962 | 1.0796 | 0.05965 | −0.0733 | 0.9054 |
| PGP \|IL23R \|ABCB9 \|CCNF \|RGMB \|GSDMC \|SLC36A3 \|LAMP5 \|GUCY2C \|ADAM20 | −0.18484244 | 0.20831 | 1.28419 | −0.0044 | −0.6506 | 0.93796 |
| SRGAP1 \|AKR1D1 \|FOXD4L1 \|PIWIL2 \|EFCAB6 \|AVPR1A \|WFDC3 \|MYO1B \|MYLPF \|ZBED2 | −0.19228922 | 0.19822 | 1.18593 | 0.03485 | −0.5402 | 0.93024 |
| CBLN3 \|POC1B-GALNT4 \|CD109 \|GPNMB \|GCHFR \|FOSL1 \|TRIM2 \|SMR3B \|FAM186A \|GABRA6 | −0.19901639 | 0.2207 | 1.21405 | 0.01248 | 0.03222 | 0.93762 |
| SHD \|USP27X \|CACNA1C \|METTL21C \|C9orf135 \|AKR1B15 \|COL5A2 \|TTLL7 \|ADH6 \|KDM4E | −0.21057685 | 0.174 | 1.17871 | 0.09552 | 0.35055 | 0.93261 |
| HAS3 \|RARRES1 \|SPC25 \|PXDN \|POTEH \|ATP9A \|MUSK \|CCDC114 \|KLHL29 \|RAB36 | −0.22144293 | 0.21824 | 0.89382 | 0.06138 | 0.97431 | 0.91929 |
| ME1 \|TIE1 \|GNG3 \|SCUBE2 \|RGS22 \|SPP2 \|ANP32D \|AXL \|RIBC1 \|SOX4 | −0.22976522 | 0.20142 | 1.10439 | 0.0714 | 0.02414 | 0.92882 |
| FAM53A \|EXOSC6 \|PURA \|PFKFB1 \|SPNS2 \|AP5S1 \|PRRT1 \|EDN1 \|CCDC81 \|KCNE1 | −0.23905492 | 0.26727 | 0.81294 | 0.03921 | 0.21544 | 0.92141 |
| FOXD4L6 \|CRYGN \|CAPNS2 \|MMP9 \|POLQ \|MCAM \|TMEM231 \|MB21D2 \|C17orf99 \|STAB2 | −0.24988767 | 0.20862 | 1.02666 | 0.06602 | 0.44126 | 0.91515 |
| PNLDC1 \|C7orf25 \|PDE10A \|IL17RB \|GNB1L \|CAMP \|HEPACAM \|ASPHD2 \|BBS12 \|SPANXD | −0.25712574 | 0.22734 | 0.91645 | 0.16925 | −0.275 | 0.91569 |
| RSPH9 \|MMP21 \|MYO3A \|BCL2L2 \|TAS2R13 \|CCDC103 \|C2orf27A \|FBLN2 \|CSMD1 \|ENPP1 | −0.26437295 | 0.2007 | 1.02129 | 0.13345 | 0.45418 | 0.92839 |
| LRP2BP \|CDCA8 \|DLX6 \|SALL2 \|RAI14 \|MICALL2 \|SORBS1 \|C8orf46 \|SPRYD4 \|OR13D1 | −0.27518573 | 0.22037 | 1.0626 | 0.07276 | −0.5576 | 0.93894 |
| PLCZ1 \|SLC6A13 \|PLCE1 \|LDHAL6B \|ANKRD1 \|FBXO15 \|STOX1 \|FLG \|GCKR \|TMEM150C | −0.28841105 | 0.15024 | 1.22719 | 0.00158 | −0.7883 | 0.93985 |
| FUT6 \|SPSB1 \|ZNF319 \|IRS1 \|NCKAP5 \|ANKLE1 \|ZNF670 \|PLCH1 \|ALDH1L2 \|BCDIN3D | −0.29962459 | 0.24497 | 1.02787 | 0.1334 | −0.484 | 0.9287 |
| RHPN2 \|IMPG2 \|FAM81A \|PPEF2 \|DNAH12 \|AOC2 \|ARHGEF25 \|CCNB2 \|TREX2 \|MT1G | −0.30837192 | 0.18585 | 1.01525 | 0.0954 | −0.031 | 0.92799 |
| RCCD1 \|PAQR4 \|SLC7A8 \|NETO1 \|FXYD4 \|SDC3 \|TEX29 \|ERCC6L \|SPRYD7 \|PRM3 | −0.31907163 | 0.22058 | 0.8962 | 0.10325 | 2.18067 | 0.90466 |
| SMC1B \|FREM3 \|ACOT11 \|INSL6 \|LRRC19 \|PRB1 \|ZNF501 \|ZNF205 \|SAMD14 \|FAP | −0.32931272 | 0.20197 | 1.33585 | −0.0104 | −0.7029 | 0.931 |
| CCDC24 \|RCVRN \|ZC3H10 \|SESN2 \|KIF5A \|GPR82 \|RIMS3 \|DPF1 \|FANK1 \|AVPI1 | −0.33620859 | 0.24484 | 0.83439 | 0.17733 | −0.3889 | 0.91619 |
| GAPDHS \|LRRN2 \|PIF1 \|IBA57 \|SOX6 \|AGRP \|CPA3 \|SLC26A3 \|SCN2A \|HES6 | −0.34485985 | 0.1824 | 1.13283 | −0.0205 | 1.42139 | 0.932 |
| MTRNR2L2 \|KCNA2 \|KREMEN1 \|CKMT1B \|EPHB3 \|CHRNA4 \|C4orf46 \|PHGR1 \|IRF2BPL \|FBXO2 | −0.35580819 | 0.23052 | 0.8804 | 0.06427 | −0.5558 | 0.90529 |
| CECR2 \|NMNAT2 \|ATP2C2 \|PON1 \|HRASLS \|C9orf40 \|B4GALT2 \|FBXO16 \|TIGD6 \|SLC2A14 | −0.36954438 | 0.20397 | 1.14712 | 0.06219 | −0.1939 | 0.93464 |
| CHIT1 \|GRIA4 \|SCN8A \|BCAR3 \|CYP27B1 \|EID2B \|CELSR1 \|KY \|KL \|OPRL1 | −0.38287909 | 0.21477 | 1.0682 | 0.02977 | −0.6578 | 0.91373 |
| C1orf146 \|DNAJC30 \|SLC13A5 \|ZNF396 \|ITLN1 \|ABCA10 \|ST6GAL2 \|ARL11 \|ITPRIPL2 \|PROZ | −0.39323922 | 0.20916 | 1.12805 | 0.0197 | −0.5198 | 0.92416 |
| SCML2 \|MMP2 \|ZCCHC3 \|ANXA3 \|MELK \|FUT4 \|CLAN1 \|FBXL22 \|FAM69B \|SMR3A | −0.40373573 | 0.22525 | 1.12002 | 0.0049 | 0.32064 | 0.93797 |
| ZNF599 \|OR3A2 \|TEKT3 \|ADAMDEC1 \|FAT4 \|CABPB \|TRPM6 \|KNG1 \|SERPINE3 \|GLRA1 | −0.41399764 | 0.18307 | 1.43988 | −0.0353 | 0.26869 | 0.94135 |
| CD1A \|SLC35G3 \|GGH \|SEMA5A \|TMEM99 \|ALDH1B1 \|MMP11 \|SYNPR \|IL36A \|STX19 | −0.42236109 | 0.20481 | 1.00881 | 0.04574 | −0.1165 | 0.92395 |
| PCP4L1 \|REEP2 \|ZNF865 \|PLS1 \|SLC6A4 \|CDCA4 \|INTU \|KRT1 \|L1TD1 \|KNDC1 | −0.43259625 | 0.20772 | 1.05032 | 0.03371 | −0.3039 | 0.9268 |
| DNAL4 \|TBC1D8B \|OR7A5 \|SLC5A10 \|ADCY10 \|AKNAD1 \|SLC3945 \|GRIA3 \|ASAP3 \|DEPDC7 | −0.44308183 | 0.19567 | 1.12531 | 0.03684 | −0.5278 | 0.93075 |
| FSIP2 \|KIAA1107 \|ARMC4 \|CCL23 \|EHF \|CFTR \|TGM4 \|RAB39B \|IGFL4 \|DAB1 | −0.44972018 | 0.15147 | 1.19752 | 0.02899 | −0.6947 | 0.94073 |
| RIMBP3C \|GINS3 \|L1CAM \|LDHAL6A \|SYBU \|PODNL1 \|SFTPB \|KRTAP16-1 \|C11orf45 \|SCNN1D | −0.46006047 | 0.19007 | 1.07341 | 0.03158 | −0.127 | 0.91053 |
| TNFSF4 \|HORMAD2 \|MCOLN3 \|C8orf31 \|ASS1 \|CABLES1 \|WNT5B \|OR2L2 \|MEGF10\|ANKRD53 | −0.47042956 | 0.1833 | 1.17127 | 0.03014 | 0.96244 | 0.92225 |
| SDC2 \|HSD17B14 \|SERPINE1 \|ITGA8 \|FFAR3 \|GRM2 \|FGF9 \|SH3RF3 \|IL27 \|TAC3 | −0.47893551 | 0.20835 | 1.11286 | 0.00889 | 0.41671 | 0.91204 |
| CDH8 \|OR2L5 \|ABCA13 \|KRTAP10-2 \|ARHGEF38 \|S100A2 \|LGI4 \|AMPD1 \|GNRH2 \|MTUS2 | −0.48649911 | 0.14835 | 1.221 | 0.04545 | −0.0772 | 0.92108 |
| MND1 \|SGCD \|ARHGDIG \|ACSBG1 \|ZNF470 \|REEP1 \|PTPRB \|AKAP14 \|PSG8 \|ZNF584 | −0.49838828 | 0.19124 | 1.01199 | 0.01093 | 1.60907 | 0.92235 |
| MGP \|PLA2G2C \|TRPC6 \|TSPAN1 \|ZNF471 \|SLC5A5 \|ZNF695 \|SKIDA1 \|RNF208 \|PDE6A | −0.50720142 | 0.19303 | 1.31533 | −0.0539 | −0.6837 | 0.92974 |
| TRIM45 \|COL1A2 \|FAM83G \|ATP1B2 \|KIF20A \|C6 \|GPLD1 \|TNNC2 \|DPYSL4 \|MMP14 | −0.51547885 | 0.21086 | 0.88321 | 0.11949 | 0.33812 | 0.91502 |
| SPINK9 \|KRT2 \|EPHX4 \|CENPM \|ANGPTL5 \|ZBTB12 \|GSG1 \|SNX31 \|BMPER \|NPM2 | −0.52579717 | 0.17143 | 1.29317 | 0.00489 | 1.41175 | 0.93331 |
| RFX4 \|SLC4A3 \|ALDOB \|BTBD8 \|SCIN \|LRFN2 \|EGFL6 \|SLC30A4 \|ARMCX2 \|NSUN7 | −0.53415231 | 0.20654 | 1.12239 | 0.07011 | 2.40279 | 0.92695 |
| DNAJB7 \|MST1R \|C1orf100 \|TNFAIP6 \|PLLP \|TSKS \|CDH13 \|VWA7 \|BAIAP2L2 \|CYP2C8 | −0.54530666 | 0.18422 | 1.27381 | 0.00879 | −0.0348 | 0.92885 |
| PCSK4 \|WISP3 \|FAM180B \|HILPDA \|RNASE10 \|CLDN12 \|KRT16 \|TUBB2B \|ASPHD1 \|LPAR5 | −0.55419965 | 0.2149 | 1.25886 | 0.03077 | −0.8179 | 0.91548 |
| HMMR \|C1orf61 \|SLC26A8 \|CDKL2 \|GCAT \|CKM \|FBXW10 \|CA4 \|GPRC5B \|RAB41 | −0.56780037 | 0.21266 | 1.02877 | 0.15873 | 0.4165 | 0.92074 |
| AICDA \|VWDE \|SCN1B \|SLC35F3 \|COL16A1 \|PCDHB12 \|C19orf18 \|RNASE1 \|ATP6AP1L \|C9orf172 | −0.58059297 | 0.18702 | 1.04583 | 0.01984 | 1.62346 | 0.92915 |
| ZNF425 \|BRI3BP \|LRRC61 \|CHI3L1 \|WFDC6 \|OPLAH \|SYDE2 \|AWAT1 \|KIAA1671 \|GNAT2 | −0.58967982 | 0.23753 | 1.07534 | 0.10433 | −0.0845 | 0.92041 |
| MARVELD2 \|PRODH \|MORC1 \|LRFN1 \|MAPK12 \|GIF \|PPP1R1A \|PLEKHH1 \|PLP1 \|HSD11B1L | −0.59520852 | 0.20475 | 0.99406 | 0.0066 | 0.50173 | 0.92656 |
| TMEM232 \|LRRN1 \|GDPD4 \|COL4A5 \|ACTA1 \|ADPRHL1 \|KRTAP21-2 \|KCNK7 \|CCDC68 \|SLC24A3 | −0.60382508 | 0.18268 | 1.1373 | −0.0005 | −0.3049 | 0.92644 |
| LILRB5 \|IRGM \|RTKN \|AQP1 \|PTK6 \|FRMD6 \|ENPP3 \|WWC1 \|PIH1D2 \|RAPGEF4 | −0.61291713 | 0.20347 | 1.0898 | 0.02457 | 1.30152 | 0.90544 |
| UPK1B \|DUSP14 \|RAC3 \|SLC47A2 \|UBTD1 \|TRIM49C \|ISM1 \|ESRRG \|GHRHR \|ACTL6B | −0.6211605 | 0.16794 | 1.17608 | 0.03295 | 0.49765 | 0.91458 |
| A2ML1 \|CPNE7 \|CHRNA5 \|F13B \|CRHR2 \|RBP1 \|TRIP13 \|FRMD4A \|HBM \|MOGAT1 | −0.63138257 | 0.20205 | 1.07813 | 0.03935 | 0.20338 | 0.92273 |
| TMEM211 \|MEP1B \|NPR3 \|CFHR2 \|RAB38 \|FAM83D \|PAH \|TMEM44 \|ZSCAN22 \|KCNH2 | −0.64314301 | 0.21191 | 0.92246 | 0.05761 | 0.54575 | 0.93698 |
| C21orf62 \|XK \|OR52H1 \|DEPDC1B \|FMO3 \|DLGAP3 \|SCD \|SPOCD1 \|ZSCAN20 \|ACOT4 | −0.65352863 | 0.21524 | 1.12855 | 0.05679 | −0.6136 | 0.93245 |
| RBPMS2 \|CCDC62 \|ASB9 \|CXCL17 \|ASPN \|NNAT \|BOLA1 \|OR1D2 \|FAM178B \|COL5A1 | −0.66366671 | 0.19463 | 1.23333 | 0.00218 | −0.0742 | 0.92959 |
| ZNF367 \|C9orf153 \|FAM109A \|C5orf64 \|XKRX \|RASSF6 \|ACTN2 \|BBC3 \|FAM83H \|FIGLA | −0.66836125 | 0.21841 | 1.12189 | 0.05409 | 0.2331 | 0.92713 |
| DDTL \|CADM4 \|SFTPD \|SLC22A13 \|ST18 \|OPRM1 \|KLHL13 \|BCAS1 \|ZCWPW2 \|SCN3B | −0.67357461 | 0.1772 | 1.12063 | 0.03243 | −0.3244 | 0.93208 |
| IGFALS \|TMEM236 \|CLEC4M \|TRHDE \|APOF \|C1QTNF6 \|C9 \|MORN4 \|TMEM31 \|ELOVL4 | −0.68199338 | 0.18945 | 1.08486 | 0.06618 | −0.9043 | 0.91529 |
| HSPA4L \|SLC8A3 \|ZAR1 \|PRICKLE4 \|SLC26A5 \|DCDC1 \|PRMT6 \|ZNF703 \|LRRTM2 \|TMEM151B | −0.68997757 | 0.22466 | 1.01682 | 0.11717 | 0.06313 | 0.92455 |
| C5orf30 \|FBXO45 \|ZNF444 \|TULP1 \|SPR \|ROBO2 \|CTSG \|PON3 \|ADH4 \|PACRG | −0.70203454 | 0.22115 | 1.16899 | 0.03401 | 1.73924 | 0.93026 |
| MMP19 \|OLFML1 \|SMIM1 \|ITIH2 \|C3orf20 \|STK32B \|LRRC71 \|FAT2 \|CCDC110 \|KCNK10 | −0.7133249 | 0.19085 | 1.21959 | −0.0181 | 1.91241 | 0.92912 |
| CDH3 \|TUBB8 \|SOGA3 \|CENPI \|GDF15 \|CXCL1 \|EGEL7 \|ETV1 \|TCF7L1 \|PLEKHG7 | −0.72092565 | 0.19169 | 1.1729 | −0.0123 | 0.03105 | 0.92433 |
| LIMS2 \|TONSL \|ANKRD24 \|TPX2 \|USH1C \|HOXA9 \|NLGN4X \|NOL3 \|RPS4Y2 \|CLEC3B | −0.72696858 | 0.22669 | 1.09652 | 0.04555 | −0.268 | 0.91453 |
| MUC15 \|DOK7 \|GTSE1 \|STAC \|CORO2B \|ADAM30 \|ZBTB42 \|SYT15 \|ECM2 \|GABRG2 | −0.73704097 | 0.18188 | 1.04554 | 0.05025 | 3.04151 | 0.91784 |
| CEACAM19 \|OR10G2 \|PRRG3 \|FRMD7 \|SPATA22 \|SALL1 \|KIF15 \|ZBTB32 \|TMEM38A \|DIAPH3 | −0.74659566 | 0.18035 | 1.17512 | 0.07502 | 4.4161 | 0.92734 |
| CYP4F2 \|C1orf194 \|CADPS \|PRR22 \|OLFML3 \|CCDC89 \|RASGEF1C \|BIRC5 \|COL1A1 \|NANP | −0.75564875 | 0.18868 | 1.23341 | 0.02269 | 1.18025 | 0.90754 |
| CLC \|GALNT8 \|DOK4 \|NR5A1 \|KCNS1 \|PDC \|PAK6 \|THSD1 \|KRTAP4-5 \|RP1L1 | −0.7666894 | 0.14398 | 1.21339 | 0.02302 | 0.44888 | 0.9185 |
| SYCP1 \|LAMA3 \|ANKRD18B \|GOLGA6C \|CLPSL1 \|FAM50B \|FA2H \|WDR64 \|POTEC \|MCM10 | −0.77389503 | 0.17285 | 1.04775 | 0.13362 | 0.71021 | 0.92829 |
| STON1-GTF2A1L \|ATP5L2 \|FAM43A \|CYP2B6 \|KANK2 \|CORIN \|CDK1 \|EPDR1 \|TRDN \|FAM9A | −0.78376074 | 0.20309 | 1.14854 | 0.05773 | 1.06642 | 0.93644 |
| IL1RL1 \|SLC18A1 \|CHADL \|RSPB1 \|ZNF71 \|FAM167A \|POTEE \|MRM1 \|LANCL3 \|TRIM40 | −0.79754763 | 0.20831 | 1.12696 | 0.06856 | −0.5682 | 0.92035 |
| GRID2 \|BGLAP \|XG \|CCR3 \|NPTXR \|MYBPC3 \|C16orf59 \|PKP3 \|HSD3B7 \|SMPDL3B | −0.81071573 | 0.19944 | 1.08714 | 0.06543 | −0.8335 | 0.90015 |
| C16orf71 \|WASF1 \|RWDD2A \|RGR \|ACMSD \|VSIG8 \|MYL2 \|MAFF \|DYTN \|CLCN1 | −0.81955951 | 0.19878 | 1.12022 | 0.01658 | −0.0453 | 0.92285 |
| ASB13 \|CCDC113 \|ACSBG2 \|OTOF \|USP50 \|CAPN14 \|CCNI2 \|PGBD4 \|SULT281 \|C4orf36 | −0.82791101 | 0.19009 | 1.06376 | 0.00929 | 0.07539 | 0.92583 |
| CLVS1 \|TSPYL5 \|ZMYND12 \|ADAM21 \|TMC3 \|DSG1 \|GRPR \|GRID2IP \|NDUFA4L2 \|AQP11 | −0.83543818 | 0.19556 | 1.21315 | 0.02335 | −0.7124 | 0.94038 |
| HSF2BP \|KHDC1 \|GPHA2 \|ZNF704 \|IL6 \|KLHL35 \|TMEM45A \|ASIP \|SLIT2 \|KHDRBS3 | −0.84551393 | 0.19898 | 1.08477 | 0.0366 | 0.29247 | 0.92781 |
| CNDP1 \|IER5L \|FOXR1 \|UNC13C \|FFAR2 \|ANKRD31 \|GDF9 \|ZFP2 \|TRHR \|CD300LD | −0.85632926 | 0.19109 | 1.13581 | 0.0854 | 0.71344 | 0.93918 |
| OR52B6 \|TRPV3 \|C1orf216 \|PTPRD \|DIRC3 \|PCDHGA10 \|KLHL4 \|LGALS13 \|SLC14A2 \|SPDYA | −0.87180922 | 0.16549 | 1.18567 | 0.02051 | 0.65027 | 0.93488 |
| PHLDA2 \|GGNBP1 \|SPESP1 \|SLC22A7 \|RBBDD3 \|LAMC3 \|SLCO4A1 \|CDHR2 \|C9orf139 \|GRIA2 | −0.88269949 | 0.21972 | 1.1432 | 0.04259 | 0.92161 | 0.89759 |
| TAS2R40 \|ZNF580 \|SLC5A4 \|C8orf82 \|SERTAD1 \|CACNA1A \|PCDHGB3 \|PTX3 \|RORC \|ALOX12B | −0.89281835 | 0.20027 | 1.20257 | 0.01844 | −0.984 | 0.92085 |
| YBX2 \|CGREF1 \|DLGAP5 \|CNTNAP3B \|TBX5 \|NLGN1 \|ZNF229 \|FHL2 \|AZU1 \|C16orf96 | −0.8980816 | 0.19483 | 1.24458 | −0.0052 | −0.315 | 0.92647 |
| UCP1 \|RAMP3 \|SHROOM4 \|ARSE \|CATSPERD \|ARMCX1 \|TAS1R3 \|GHRH \|POU6F2 \|CYP2A7 | −0.90835059 | 0.18457 | 1.14306 | 0.05037 | 0.91447 | 0.92167 |
| OR56B1 \|CNFN \|GNG7 \|C20orf197 \|PLCB4 \|PCDHGA7 \|DCST1 \|CENPA \|KIF7 \|ACSM5 | −0.91930858 | 0.19326 | 1.04455 | 0.03435 | −0.3343 | 0.9261 |
| DGAT2L6 \|BRSK2 \|GPR27 \|DLG2 \|HBQ1 \|C22orf15 \|NBL1 \|C6orf99 \|SALL4 \|HIC1 | −0.93110925 | 0.21104 | 1.2732 | 0.0046 | −0.6911 | 0.91648 |
| NOL4 \|TMEM51 \|GPR146 \|VN1R1 \|CALB2 \|SLC28A2 \|DQX1 \|RLN3 \|XPNPEP2 \|LMCD1 | −0.9415301 | 0.18469 | 1.1647 | 0.06887 | −0.6717 | 0.91892 |
| DPP10 \|RGS17 \|RAP1GAP \|EPX \|C1orf87 \|OR2AE1 \|KCNV2 \|OR4D1 \|KRTAP10-3 \|PCDHGB7 | −0.95839746 | 0.15977 | 1.18504 | −0.031 | −0.1039 | 0.92956 |
| SYCP2L \|C15orf48 \|TEX14 \|SORBS2 \|CLCNKA \|VWA3B \|ZNF784 \|LRRC56 \|ZNF835 \|F12 | −0.97233306 | 0.2314 | 1.02445 | 0.05672 | 0.66755 | 0.92623 |
| VASN \|HSD17B6 \|ADAMTS4 \|DNAJC28 \|CXCL12 \|GALNT13 \|CAP2 \|SRSF12 \|TRIM7 \|C17orf100 | −0.98205388 | 0.2365 | 0.99505 | 0.14929 | −0.2237 | 0.91624 |
| SDK1 \|CPA4 \|WNT1 \|SVOPL \|PCDHB4 \|ZNF284 \|F3 \|PTGES3L \|C4orf19 \|RASGRF1 | −0.99608353 | 0.20775 | 1.07052 | −0.0305 | 5.52199 | 0.93031 |
| CXCL2 \|USP2 \|TTC23 \|POU5F2 \|KCND1 \|NR1I2 \|BTNL8 \|PPEF1 \|GLRB \|FBXO48 | −1.00858409 | 0.20571 | 1.2228 | 0.03167 | −0.6841 | 0.93547 |
| RNF151 \|CELF4 \|ERG \|TOX2 \|GNG10 \|MS4A6E \|DDI1 \|TTC23L \|SPINK7 \|TGFA | −1.01726124 | 0.2221 | 1.14391 | 0.02107 | −0.0474 | 0.93117 |
| IFNA21 \|ABCC8 \|COL27A1 \|IQCF1 \|ELE5 \|APBA1 \|SELENBP1 \|KRT86 \|FAM124B \|F11 | −1.02741732 | 0.16451 | 1.2259 | −0.0132 | 1.49873 | 0.92819 |
| PTCH2 \|MZT1 \|DNAH8 \|OR13C5 \|CYP11B1 \|AR \|WDR86 \|DNASE1L2 \|SH2D4A \|TMEM244 | −1.03673324 | 0.16404 | 1.31548 | −0.0524 | 0.18036 | 0.92806 |
| FAM47E \|WEE2 \|FAM149A \|NAALADL2 \|ALPK2 \|RHOXF1 \|KIF24 \|PDE6H \|BTNL2 \|NRIP3 | −1.04682111 | 0.18572 | 1.00984 | 0.04614 | −0.4327 | 0.94112 |
| MEIG1 \|KIF4A \|SOX12 \|NTN4 \|POU5F1B \|FSIP1 \|C8orf87 \|FGF2 \|JAG2 \|RGPD4 | −1.05807887 | 0.2192 | 1.06667 | −0.0262 | −0.9901 | 0.93666 |
| ZFHX2 \|CYTL1 \|POTED \|TRIM47 \|COL28A1 \|BATF2 \|POF1B \|IL26 \|IQCA1 \|TMOD1 | −1.07002213 | 0.19792 | 1.08121 | 0.01909 | −0.6662 | 0.93385 |
| AP1M2 \|CYP46A1 \|SULF1 \|SLC45A1 \|FGF11 \|NXN \|COL11A1 \|CCDC83 \|CSRP3 \|CELA1 | −1.0809639 | 0.20564 | 1.05885 | 0.02869 | 0.7462 | 0.92397 |
| PABPN1L \|OR13C9 \|POTEB \|ADCY5 \|WWTR1 \|SPATC1 \|SLC26A7 \|HBE1 \|AADAT \|FKBP1B | −1.0909091 | 0.17829 | 1.13285 | 0.01611 | 1.64985 | 0.9339 |
| SPATA9 \|DYNC1I1 \|EFHB \|PGPEP1L \|HCN3 \|OR52N1 \|TAS2R4 \|RSPO4 \|RBFOX1 \|SKOR1 | −1.09963226 | 0.14289 | 1.28526 | −0.0427 | 0.26342 | 0.933 |
| ZCCHC24 \|MAST1 \|POTEF \|RAPGEF5 \|KIF14 \|NAALAD2 \|SAMD15 \|CSH1 \|DCT \|TCP10L | −1.10780299 | 0.20891 | 1.08076 | 0.03824 | 2.32371 | 0.93234 |
| OPTC \|CGB2 \|SPRR2B \|NOXRED1 \|CALCA \|ZSCAN23 \|E2F7 \|TLE6 \|ODF3 \|MACC1 | −1.12293943 | 0.17121 | 1.27382 | −0.016 | −0.1638 | 0.9288 |
| KRTAP20-1 \|SOAT2 \|CELSR3 \|RASEF \|CCDC73 \|TRMT61A \|SNAP91 \|C10orf91 \|IGLL1 \|KIF1A | −1.13470806 | 0.18488 | 1.24433 | 0.04217 | −0.778 | 0.91089 |
| PPP1R42 \|PNPLA3 \|SPHK1 \|SCG5 \|TSPYL6 \|C4orf51 \|DRD2 \|GPR84 \|LREN4 \|TBX1 | −1.14216761 | 0.1977 | 1.07578 | 0.04446 | −0.5596 | 0.92191 |
| FAT1 \|MS4A5 \|PIGR \|ZNF479 \|TNFSF11 \|APC2 \|CAV2 \|DOK6 \|NTN5 \|HSPB3 | −1.16141139 | 0.15297 | 1.30397 | −0.0049 | −0.3957 | 0.9208 |
| ZNF99 \|TAS2R10 \|APLP1 \|C1orf186 \|PCDHGB6 \|ZNF729 \|C7orf34 \|PCDHGA6 \|SHANK1 \|C8orf37 | −1.17037036 | 0.17144 | 1.24259 | 0.03079 | −0.7785 | 0.93478 |
| SV2A \|ZNF878 \|ADCY2 \|C2orf15 \|MNS1 \|ETV2 \|P2RY4 \|USP51 \|C19orf57 \|KRTAP4-12 | −1.18202432 | 0.22227 | 1.02004 | 0.09069 | −0.9212 | 0.92594 |
| SBSPON \|RFX6 \|FAIM2 \|TMEM114 \|SEC16B \|ALPL \|DDX4 \|ASPG \|TBC1D16 \|ZNHIT2 | −1.19206134 | 0.18855 | 0.924 | 0.02219 | 0.527 | 0.91489 |
| ZNF98 \|FOXC1 \|CACNA1H \|IGFBP6 \|CDH24 \|ARHGEF26 \|MLLT11 \|MAGEA2B \|PDE1C \|FOXI1 | −1.20397184 | 0.23787 | 1.09681 | 0.08289 | −0.7101 | 0.90992 |
| TANC1 \|C3orf33 \|MANSC4 \|KRTAP19-3 \|CLUL1 \|ABRA \|DOC2B \|TMPRSS2 \|SLC28A3 \|SLC25A34 | −1.21895207 | 0.17509 | 1.08684 | 0.05556 | −0.488 | 0.92489 |
| ROR1 \|OBSL1 \|KRT81 \|P2RX2 \|TRIM55 \|GINS1 \|GP5 \|COL25A1 \|LRRC46 \|CRYBB3 | −1.22691552 | 0.20031 | 1.31091 | −0.017 | 1.64974 | 0.92943 |
| TMC1 \|GNB3 \|PPP1R26 \|NRXN1 \|MUC13 \|IL12A \|HDHD3 \|WDR72 \|MYLK3 \|HES1 | −1.23395019 | 0.18282 | 1.07325 | 0.01848 | 0.07328 | 0.91865 |
| TPTE2 \|CCDC116 \|SDC4 \|LRRIO1 \|PRRX1 \|MAPK11 \|HIST3H3 \|C4orf22 \|CHST4 \|MIPOL1 | −1.2439898 | 0.20593 | 1.19978 | −0.0072 | −0.0497 | 0.93496 |
| KRT3 \|CHRNA10 \|CYP2J2 \|ADAMTSL3 \|SCD5 \|HPCAL4 \|BEND7 \|PLVAP \|AQP4 \|C19orf25 | −1.25623148 | 0.1834 | 1.13526 | 0.0513 | 0.42127 | 0.91608 |
| KRTAP5-10 \|FOXL1 \|NGEF \|LDHD \|CEP55 \|USP6 \|PCDHB2 \|PAIP2B \|PLAT \|C4orf48 | −1.26870027 | 0.18164 | 1.17688 | 0.0487 | 0.63136 | 0.91809 |
| C20orf173 \|CGB5 \|SERP2 \|TP73 \|GPR39 \|PRSS37 \|BPIFC \|DCN \|SLC44A3 \|C3orf30 | −1.27545244 | 0.15515 | 1.11281 | 0.03301 | 1.01381 | 0.9266 |
| GALNT14 \|FAM184B \|PDLIM4 \|ARL5C \|FZD3 \|ZNF579 \|FCRLB \|CNR2 \|CKAP2L \|SLC44A4 | −1.28396418 | 0.19647 | 0.93565 | 0.13425 | −0.6034 | 0.91685 |
| SCG3 \|SLC47A1 \|FGFR3 \|EPHX3 \|FUT3 \|CEBPE \|C6orf226 \|C19orf35 \|PRSS22 \|METRN | −1.29410772 | 0.21935 | 1.18904 | 0.0723 | 0.60628 | 0.89869 |
| CDCA3 \|MEIS2 \|NHLRC1 \|SIX5 \|MSH4 \|KIAA1614 \|PPP1R36 \|OLR1 \|AMBP \|VASH2 | −1.30564504 | 0.22283 | 1.24655 | 0.00601 | −0.2522 | 0.93288 |
| GRIK5 \|FBXO36 \|GRIN3B \|CNN1 \|KRBOX1 \|AMN \|OR2F1 \|SI \|SLC4A9 \|CFC1 | −1.31531594 | 0.18003 | 1.17492 | 0.00911 | 2.15386 | 0.92525 |
| RBMY1A1 \|GREB1 \|FZD1 \|SOBP \|HTR4 \|CDKN2B \|ZNF157 \|SAMD13 \|CEI \|C2orf82 | −1.3222114 | 0.17534 | 1.0931 | 0.05438 | −0.6261 | 0.9344 |
| FOLR1 \|NXF2B \|MOV10L1 \|IRF6 \|PAGE4 \|NGF \|CCDC148 \|LRP1B \|GPRASP2 \|OR1K1 | −1.33162895 | 0.19757 | 1.21468 | −0.0028 | −0.828 | 0.9207 |
| MID1 \|SLC16A2 \|CCDC151 \|PCDHB16 \|ZNF541 \|HFE2 \|OR52D1 \|FAM182B \|GSTM5 \|UCHL1 | −1.33828917 | 0.19485 | 1.19716 | −0.0417 | −0.446 | 0.93059 |
| ACER1 \|ACRV1 \|DNAJC25-GNG10 \|TSNAXIP1 \|ARL4D \|EMX2 \|GATA6 \|KCNQ3 \|ID1 \|NFASC | −1.34926814 | 0.21236 | 0.94441 | 0.14395 | 1.75123 | 0.92123 |
| NTRK3 \|ARHGEF33 \|TP53TG3C \|PKDREJ \|PCDHGB2 \|ILDR2 \|TYRO3 \|ZP1 \|MOXD1 \|SCRG1 | 1.36026022 | 0.20371 | 1.15887 | −0.0392 | −0.9505 | 0.92981 |
| KIAA1211 \|C8orf34 \|IGSF11 \|OR2H2 \|PDF \|INMT \|DOCK6 \|ZNF391 \|CYLC2 \|PRSS27 | −1.36881699 | 0.20741 | 1.1807 | 0.0227 | −0.0479 | 0.91913 |
| TCEAL5 \|CMTM8 \|CDH9 \|MMEL1 \|VCAM1 \|TFAP2A \|SLC6A2 \|DSCC1 \|TCAP \|LIF | −1.37840319 | 0.19187 | 1.08234 | 0.11018 | −0.1286 | 0.91265 |
| SLC36A2 \|MYEOV \|MUC16 \|ESPL1 \|HHIP \|ZNF667 \|CRYAB \|GRIN3A \|SHANK2 \|OCA2 | −1.38720594 | 0.17691 | 1.0216 | 0.06335 | −0.1029 | 0.91234 |
| PARVA \|PKMYT1 \|SCN4B \|TEX28 \|SLC45A2 \|KLK1 \|E2F1 \|MLN \|ABCA8 \|CDH20 | −1.4025913 | 0.17542 | 1.165 | 0.06644 | 0.29169 | 0.9175 |
| RFPL2 \|CASQ1 \|PCDHGA9 \|HRASLS5 \|SLC7A11 \|BOLL \|CLGN \|LDLRAD2 \|CLDN11 \|DEPDC1 | −1.41150583 | 0.2031 | 1.19605 | 0.01729 | 1.2163 | 0.93175 |
| RHBDF1 \|CDCA5 \|SHH \|KCNK18 \|OR1E1 \|TSSK3 \|ASPA \|ZC3HAV1L \|CCDC71L \|RASD1 | −1.41669205 | 0.20515 | 1.02422 | 0.05121 | 1.92841 | 0.92192 |
| AHRR \|SUSD2 \|MYO16 \|PTPN21 \|A1CF \|ATOH8 \|RNF17 \|FBLN1 \|ABCA4 \|ANKUB1 | −1.4249812 | 0.16096 | 1.17993 | −0.0013 | 1.33261 | 0.93473 |
| DDAH1 \|HESX1 \|ACOXL \|DLEU7 \|TMPRSS15 \|MAL2 \|GABRE \|CCDC80 \|C16orf46 \|TMEM163 | −1.43691451 | 0.19853 | 1.04852 | 0.06517 | 0.05206 | 0.93168 |
| LCN12 \|PRSS53 \|C10orf105 \|HCN2 \|MRC2 \|C1orf53 \|LENEP \|GABRG3 \|PLSCR2 \|DKK4 | −1.44789679 | 0.1803 | 1.10864 | 0.02455 | 1.32199 | 0.90927 |
| ACSM2A \|TRPM8 \|WNT2B \|KCNMB4 \|TBPL2 \|PSORS1C1 \|VIPR2 \|PDZD7 \|TMEM190 \|KCNA5 | −1.45902594 | 0.18572 | 1.23474 | −0.0395 | −0.2338 | 0.91107 |
| TDRD1 \|DDO \|TCEAL2 \|CHD5 \|BSPRY \|TAS1R1 \|WTIP \|SGIP1 \|C2orf40 \|C4orf32 | −1.46683811 | 0.19213 | 1.10962 | 0.06022 | −0.7601 | 0.92118 |
| SLC6A15 \|BICC1 \|PRSS36 \|PNMT \|KLBL26 \|CCL14 \|GFAP \|PDZRN3 \|GGTLC2 \|NPW | −1.4733187 | 0.1788 | 1.10203 | 0.08753 | 0.49683 | 0.90792 |
| ADCYAP1R1 \|MYPN \|MLIP \|RAB3C \|KRT17 \|FSD2 \|NR2F6 \|SPACA7 \|RHBDL1 \|KRT24 | −1.48170836 | 0.17429 | 1.20524 | 0.04562 | −0.428 | 0.92914 |
| C3orf52 \|EYA1 \|EYA4 \|MYRIP \|SPRED3 \|POMC \|HSPB9 \|OR1J1 \|BEST2 \|SCGB1C1 | −1.49308679 | 0.19472 | 1.11351 | 0.02525 | 4.22611 | 0.91717 |
| CSN1S1 \|KONH1 \|ABCG8 \|STEAP1B \|GXYLT2 \|SCN5A \|PPFIA2 \|TTLL9 \|ERC2 \|PLEKHH3 | −1.50425813 | 0.20279 | 1.02317 | 0.00252 | 1.29058 | 0.92233 |
| RASL11B \|CASQ2 \|APOA1 \|C11orf42 \|PRR15L \|AMHR2 \|C4orf17 \|PTPRG \|EPCAM \|GAD1 | −1.52030477 | 0.16227 | 1.18631 | 0.01298 | 0.40529 | 0.9238 |
| VSNL1 \|TIGD3 \|DDC \|DLGAP1 \|DAO \|GPC5 \|EXOC3L1 \|LCAS \|NAV3 \|TMEM132B | −1.53089898 | 0.17834 | 0.94195 | 0.11217 | 0.39401 | 0.93172 |
| HOXB7 \|TMEM72 \|PROM2 \|TPD52L1 \|TSPAN19 \|GOLGA7B \|C1QTNF1 \|ANKS1B \|RND1 \|ANO7 | −1.54241425 | 0.18322 | 1.14453 | 0.07538 | −0.5507 | 0.91297 |
| TMEM30B \|PDCD1LG2 \|ZNF215 \|SLC39A2 \|PACSIN3 \|LRRC4B \|VWA3A \|MET \|ACTG2 \|TMPRSS9 | −1.55434005 | 0.17354 | 1.14422 | 0.07242 | −1.0116 | 0.9239 |
| CACNG1 \|C10orf113 \|GRIN2B \|MYBPC2 \|PXDNL \|LGALS14 \|ACTL7A \|PLAC8L1 \|NBPF6 \|CHRNA3 | −1.5656093 | 0.14353 | 1.31131 | −0.0592 | −0.8011 | 0.92981 |
| C17orf50 \|TMEM52 \|FOXD4 \|PCDHGB4 \|MOCS3 \|C12orf54 \|MS4A3 \|ROPN1 \|PDPN \|IL17B | −1.57749568 | 0.18235 | 1.11316 | −0.0027 | 1.80016 | 0.92761 |
| ZNF781 \|PRKAG3 \|C4orf26 \|KRT6B \|CBY3 \|SLC30A3 \|ZNF556 \|PTH2R \|SLCO5A1 \|RAG1 | −1.58671828 | 0.17961 | 1.333 | −0.0017 | 0.45828 | 0.9293 |
| SLC34A2 \|TFCP2L1 \|DEPDC4 \|JPH3 \|TMCO2 \|ZNF311 \|PCDHGC5 \|DSCR8 \|SPANXA1 \|SPANXA2 | −1.59639047 | 0.16647 | 0.94151 | 0.01481 | −0.9835 | 0.93084 |
| SVOP \|MAP1B \|ADAMTSL5 \|TMEM178A \|PROM1 \|CHRM5 \|GSTT2B \|CRYGC \|KRT78 \|ANKRD33B | −1.60715205 | 0.17358 | 1.18639 | 0.0101 | 3.19715 | 0.92566 |
| APOE \|AK7 \|C2orf16 \|PLSCR4 \|PRKCG \|PARS2 \|RUNDC3A \|OR9A4 \|PRRT2 \|VN1R2 | −1.61618401 | 0.21746 | 1.04848 | 0.0557 | −0.0887 | 0.92142 |
| C12orf60 \|KCNN3 \|IGIP \|FREM1 \|ART3 \|PLEKHN1 \|KBTBD12 \|HMCN1 \|SEMA3E \|H3F3C | −1.62386979 | 0.18799 | 1.13557 | 0.05502 | −0.2286 | 0.9419 |
| PTH1R \|TTLL2 \|SLC13A3 \|LPAR3 \|GJC3 \|IGSF23 \|KRTDAP \|C17orf51 \|NRSN2 \|ARPP21 | −1.63192482 | 0.17298 | 1.06438 | 0.07539 | 1.33362 | 0.92829 |
| NIPAL1 \|P2RX6 \|C7 \|PTPRU \|KDELC1 \|EPN3 \|CDS1 \|DSEL \|HEY2 \|NTM | −1.6477455 | 0.22957 | 1.04233 | 0.06086 | 2.80825 | 0.92319 |
| CDC25C \|HFM1 \|ABTB2 \|MEOX2 \|NRL \|FAM189A1 \|TFAP2B \|TPPP2 \|C9orf43 \|GEM | −1.65527392 | 0.22917 | 0.93894 | 0.10798 | 2.27255 | 0.93279 |
| MAB21L2 \|STC2 \|LAMA1 \|TICAM2 \|ZNF774 \|MPP3 \|CHP2 \|SBSN \|PXDC1 \|NPNT | −1.66478268 | 0.20189 | 1.01922 | 0.074 | 1.83491 | 0.91659 |
| PDE11A \|MMP8 \|RDH16 \|LHX9 \|UGT2B10 \|DYRK3 \|NCS1 \|MFSD6L \|TIGD7 \|SLC10A6 | −1.67758724 | 0.20496 | 1.00702 | 0.03391 | −0.135 | 0.92751 |
| XIRP2 \|SLFNL1 \|TRIM49B \|GPR20 \|KLHDC8A \|FRG2B \|AGMO \|ARHGAP44 \|CRB2 \|ATP8A2 | −1.68934695 | 0.1407 | 1.10626 | 0.01587 | 0.21865 | 0.92459 |
| NR4A3 \|ERN2 \|KRT8 \|IGFBP4 \|KIAA1522 \|NALCN \|SLC12A8 \|PIP5KL1 \|ELOVL3 \|KRTAP4-6 | −1.70238524 | 0.20115 | 1.06264 | 0.01535 | −0.4178 | 0.912 |
| GIPC3 \|OXT \|CDC42EP4 \|HSD17B2 \|TMEM121 \|LRG1 \|NEK2 \|LRIG3 \|TGIF2LY \|ANTXR1 | −1.7159429 | 0.23814 | 1.02502 | 0.11709 | 0.76558 | 0.91772 |
| LRRTM4 \|IL2 \|HHIPL2 \|TROAP \|C16off89 \|KIF17 \|AJAP1 \|IQCJ-SCHIP1 \|CCL8 \|TBX20 | −1.72455715 | 0.17254 | 1.15972 | −0.0102 | −0.5682 | 0.93356 |
| GPR19 \|TMEM108 \|B3GNT3 \|ADAM11 \|H1FOO \|PIEZO2 \|PCOLCE2 \|PAPPA \|TBATA \|CHSY3 | −1.73073632 | 0.18601 | 1.06902 | 0.05321 | −0.3526 | 0.91157 |
| KLHL33 \|CAGE1 \|TSPY1 \|CYP2C9 \|DSG2 \|GPRC5C \|ZNF732 \|CYP3A43 \|TH \|EXD1 | −1.74069463 | 0.18935 | 1.25994 | 0.00968 | −0.563 | 0.92537 |
| CSN3 \|FHOD3 \|ADO \|PCDHGA11 \|GLT8D2 \|EYA2 \|ART1 \|C8G \|UCN \|OR2L3 | −1.7510266 | 0.1904 | 1.15404 | −0.0119 | −0.0865 | 0.92971 |
| EFR3B \|WFDC12 \|LCE1D \|GABRA1 \|SPINK1 \|ZBBX \|C2orf80 \|ATP4B \|CAV1 \|C22orf46 | −1.76237844 | 0.16516 | 1.28135 | −0.0446 | 0.85876 | 0.92732 |
| ENDOU \|TEDDM1 \|GYS2 \|MPP2 \|GPC3 \|NLRP9 \|FAM110B \|AOC3 \|UGT2A3 \|ZDHHC19 | −1.77434944 | 0.15454 | 1.29846 | 0.01173 | 1.37215 | 0.92467 |
| RAMP1 \|DUSP4 \|CT45A1 \|FGF20 \|TIMP3 \|AKR1C4 \|SERPINA10 \|CEACAM8 \|HOXA4 \|SDCBP2 | −1.78614626 | 0.19252 | 1.05833 | 0.0221 | −0.6506 | 0.92051 |
| GSTA3 \|GJA4 \|CPNE6 \|SUN3 \|CT47B1 \|KRTAP4-4 \|SMAD6 \|OR56B4 \|SH2D5 \|C1orf68 | −1.79781948 | 0.18101 | 1.24384 | 0.01439 | 0.28666 | 0.9269 |
| RBM20 \|PRRG2 \|SPDYC \|ZNF165 \|TIFAB \|GLYATL1 \|OR8G5 \|SKA1 \|CDCA2 \|KRTAP19-7 | −1.81187028 | 0.2125 | 1.14343 | 0.04623 | 0.07621 | 0.93903 |
| SPRR2E \|PPP1R3A \|SCN1A \|KLBL25 \|KCNQ4 \|CTAGE1 \|KRTAP10-12 \|SNCG \|HTR3E \|TMPRSS4 | −1.82179734 | 0.16086 | 1.02401 | 0.01994 | 3.05152 | 0.92852 |
| MAGIX \|CENPBD1 \|HUNK \|PLEKHG4B \|TMEM74B \|ALOX15 \|SLC15A1 \|IL1RAPL1 \|WNTSB \|UFSP1 | −1.83411919 | 0.19908 | 1.03226 | 0.03192 | −0.0614 | 0.91612 |
| ADAT3 \|CES3 \|PLAU \|TEKT2 \|LMO3 \|NOSTRIN \|TDRD12 \|RAET1G \|GABRB1 \|ZNF497 | −1.84786761 | 0.22083 | 1.11064 | 0.08318 | −0.5585 | 0.92307 |
| HNF1A \|LCT \|RNASE8 \|NECAB2 \|TEX35 \|C17orf78 \|OR8B2 \|PCDHGA4 \|NEK5 \|TNR | −1.85389835 | 0.15999 | 1.19485 | 0.0144 | −0.3708 | 0.93157 |
| ATP1A2 \|FST \|ATP6V0A4 \|CELA3A \|SLC5A12 \|ISL1 \|KRT76 \|OR52W1 \|CXCL3 \|S100G | −1.86211376 | 0.1699 | 1.2001 | −0.0324 | −0.5185 | 0.92141 |
| SAMD11 \|DBH \|TEKT1 \|DMRT2 \|USHBP1 \|SEMA3D \|TSPAN12 \|UGT1A8 \|ANKRD34A \|BMPR1B | −1.87026751 | 0.16512 | 1.24923 | 0.01046 | 0.98199 | 0.93458 |
| GBP6 \|LPO \|SLC23A3 \|THPO \|FAM151A \|SYNPO2 \|TCHHL1 \|CLDN3 \|ITGA11 \|MMRN2 | −1.88284731 | 0.15922 | 1.25922 | 0.01684 | 0.28571 | 0.91805 |
| ERBB4 \|LRRC17 \|MBLAC1 \|SPATA17 \|KSR2 \|LCN8 \|EHD2 \|CPO \|KCNJ3 \|PEBP4 | −1.89233495 | 0.19136 | 1.26569 | 0.02364 | 1.51319 | 0.92614 |
| FRAT2 \|ADH1A \|ADAMTS5 \|LNP1 \|OR3A3 \|ONECUT1 \|CELA2A \|THY1 \|HTR7 \|LGSN | −1.89854982 | 0.19162 | 1.05192 | −0.0074 | −0.8669 | 0.9329 |
| SLC25A53 \|CTSE \|PRAMEF1 \|GCM2 \|CFHR3 \|WNK2 \|PCDHGB1 \|SP5 \|SLC22A3 \|NPAS3 | −1.91365307 | 0.16957 | 1.25501 | −0.0133 | −0.7797 | 0.92422 |
| OPN1LW \|SUSD5 \|TNFRSF12A \|TMEM174 \|SYDE1 \|DNTT \|CCDC169 \|PLD6 \|OLIG1 \|CLDN25 | −1.92630383 | 0.20883 | 1.15831 | 0.01927 | 2.13822 | 0.92888 |
| KRT7 \|FCN2 \|LDB3 \|PTPN3 \|CPT1C \|TEX12 \|BHMT2 \|SOX15 \|SULT1C4 \|PRDM16 | −1.93970063 | 0.16664 | 1.11424 | 0.00597 | 0.61479 | 0.91301 |
| BAMBI \|CFHR4 \|XKR9 \|HMSD \|KCTD11 \|DNAH5 \|PRSS35 \|CNTN1 \|EMX1 \|GPR179 | −1.95145721 | 0.20995 | 1.07571 | 0.07676 | 0.72802 | 0.92984 |
| CSF2 \|SHB \|C1orf127 \|CCDC155 \|FXYD3 \|RAX \|PRSS50 \|SLC2A10 \|EFNA1 \|MN1 | −1.96444451 | 0.20271 | 1.18831 | 0.0455 | −0.6287 | 0.89979 |
| LDHC \|TRIM31 \|GABRR1 \|HHLA2 \|CNRIP1 \|TRIM48 \|TEKT5 \|OR1F1 \|ZNF70SD \|IL17RD | −1.97451119 | 0.16486 | 1.20515 | −0.0279 | 0.58924 | 0.93969 |
| TNFRSF11B \|SPIN4 \|SRPX2 \|CNGA4 \|SPZ1 \|WNT11 \|HRG \|ADARB2 \|MAPK15 \|UGT2B28 | −1.98443999 | 0.18329 | 1.14098 | 0.02863 | 2.01999 | 0.93487 |
| OMP \|TRPV5 \|TEK \|ARHGAP28 \|NPPB \|UGT2B15 \|PRPH2 \|FBXL16 \|SOHLH2 \|KCNK9 | −1.99826547 | 0.15416 | 1.20412 | −0.0102 | −0.278 | 0.91871 |
| S100P \|FKBP10 \|EML1 \|DPYS \|SLCO2A1 \|COL8A1 \|C17orf58 \|S100A7 \|ACCSL \|GLTPD2 | −2.01083509 | 0.19846 | 1.18233 | 0.02572 | 4.59456 | 0.92004 |
| PNMAGA \|TARM1 \|CA12 \|SYN1 \|LOH12CR2 \|MLXIPL \|CADM2 \|DUSP28 \|CALCR \|FSHR | −2.02315142 | 0.21852 | 1.11124 | 0.02193 | 0.11536 | 0.93647 |
| MAB21L3 \|GLDN \|UBXN10 \|COLEC11 \|SLC1A2 \|GJB6 \|APOC4 \|VIT \|CA7 \|SSTR3 | −2.02979958 | 0.14982 | 1.05592 | 0.04576 | 5.21723 | 0.91949 |
| CNTN2 \|TECRL \|ATP4A \|ZNF385C \|BLOC1S3 \|GADD45G \|TOR4A \|EBF3 \|SFN \|KAZN | −2.03757434 | 0.20906 | 1.05256 | 0.00302 | 0.98201 | 0.90958 |
| FAM124A \|ROPN1L \|NUPR1 \|ATAD3C \|CHPF \|LRRC66 \|GRXCR1 \|PKD1L1 \|PCDHAC2 \|PITPNM3 | −2.04822589 | 0.19743 | 1.14616 | 0.02654 | −0.1642 | 0.93254 |
| PDE1A \|ALB \|SYNC \|REG4 \|PGAM2 \|CAMSAP3 \|XAGE3 \|ZDHHC1 \|MYT1L \|NAT8 | −2.05832139 | 0.18261 | 1.23867 | 0.00381 | −0.0458 | 0.93182 |
| PLIN1 \|DPP6 \|SNAI1 \|C2orf91 \|IDO2 \|C10orf25 \|OR1N1 \|TMEM200A \|LYZL6 \|DEFB129 | −2.06630307 | 0.15958 | 1.27573 | 0.02312 | 0.69175 | 0.93366 |
| RP1 \|COL4A1 \|GPR143 \|PCDHGA8 \|ZNF334 \|RIMS2 \|CXCL11 \|GGN \|ATP13A5 \|SHISA9 | −2.07924507 | 0.19673 | 1.26734 | −0.0318 | −0.1439 | 0.9372 |
| USP43 \|DYNLRB2 \|VSTM2B \|PPY \|TMEM212 \|TERT \|RNASE7 \|SEC14L3 \|IBSP \|TPSAB1 | −2.0969381 | 0.14455 | 1.10676 | 0.03567 | 1.79007 | 0.91726 |
| SYNE4 \|SLIT1 \|PCDHGA5 \|ANKRD35 \|HOXA3 \|APOBEC4 \|PRL \|TMEM171 \|UGT3A2 \|ALDH1L1 | −2.11435692 | 0.16703 | 1.23408 | −0.0182 | −0.0169 | 0.92755 |
| NMU \|RRAD \|SEMA3G \|C6orf52 \|LMOD1 \|TAT \|TTC30A \|PTGER3 \|OSBPL6 \|CNBD1 | −2.12041328 | 0.21592 | 1.10072 | 0.08712 | −0.3511 | 0.92731 |
| FABP12 \|SH2D4B \|MMP1 \|KIAA1217 \|NANOGNB \|LAMA4 \|OSTN \|IGSF5 \|OR10H1 \|MYLK4 | −2.12887811 | 0.13444 | 1.37258 | −0.0414 | 0.90S52 | 0.9375 |
| FSTL4 \|GCK \|MAEL \|CYYR1 \|GPR87 \|OTUD7A \|PCDHB10 \|CALY \|PHKA1 \|CCDC85B | −2.14020706 | 0.18618 | 1.08285 | 0.04107 | −0.3458 | 0.93142 |
| OR8S1 \|HMGA2 \|KLF1 \|SLC5A1 \|TMEM217 \|NOVA1 \|PHF21B \|CFHR1 \|AK8 \|ITGB8 | −2.14478707 | 0.20836 | 1.20927 | −0.0075 | 1.16249 | 0.91888 |
| TAF7L \|CARD10 \|SLURP1 \|CCR10 \|FOXN1 \|GJC2 \|FAM217A \|SNX33 \|FMO2 \|ITPKA | −2.15366411 | 0.18152 | 1.12837 | −0.0025 | 0.14901 | 0.90413 |
| MTRNR2L3 \|TCTEX1D4 \|MSI1 \|EVPL \|CRP \|GALNTL6 \|SLIT3 \|VSIG10L \|CYP2C19 \|SEMG2 | −2.16662124 | 0.17942 | 1.21729 | 0.00121 | −0.2214 | 0.92321 |
| UNC45B \|SRY \|KLK12 \|LHCGR \|SLC29A4 \|LOXL1 \|BMP8B \|SPIC \|CABP1 \|CYR61 | −2.17600012 | 0.18554 | 1.02245 | 0.0481 | 1.1991 | 0.91272 |
| KCNK13 \|CREB3L3 \|SNCAIP \|C12orf40 \|AGBL4 \|ST8SIA5 \|ADAMTS18 \|SEL1L2 \|IFNA13 \|FOXD2 | −2.18441478 | 0.17939 | 1.23816 | 0.01309 | 1.286 | 0.92825 |
| CD207 \|SIX3 \|MAGI2 \|DGKI \|TGM5 \|OR1G1 \|ENTPD2 \|ACSM2B \|LGI1 \|PERP | −2.19769474 | 0.16505 | 1.07458 | 0.00075 | 1.5426 | 0.92183 |
| CDHR4 \|GPC6 \|GPR135 \|VSTM2A \|VEGFC \|SLCO6A1 \|DENND2A \|DKK2 \|LOX \|CDC25A | −2.21068875 | 0.18601 | 1.0461 | 0.01711 | 0.94214 | 0.92506 |
| ABCC9 \|CST6 \|SV2C \|CDH17 \|FAM9C \|FGFBP3 \|C7orf71 \|C4orf45 \|RIT2 \|RNASE11 | −2.22158195 | 0.1591 | 1.31002 | −0.0532 | 0.62621 | 0.9452 |
| CSH2 \|MPDZ \|PBK \|NXF3 \|TMPRSS7 \|BCL6B \|RAB40A \|TBX3 \|NKAPL \|C16orf95 | −2.23563231 | 0.21343 | 1.17689 | 0.00994 | 0.34557 | 0.93476 |
| IL22 \|C7orf69 \|PTPRN2 \|TRIM9 \|CCDC36 \|ESRP1 \|FAM159B \|FMOD \|P2RY1 \|PCDH17 | −2.24474051 | 0.18324 | 1.34474 | −0.0637 | 0.85301 | 0.93149 |
| TCERG1L \|CTNND2 \|GAL3ST1 \|RPS6KA6 \|POU2F3 \|RNF112 \|KRT74 \|SLC34A1 \|NTRK2 \|C16orf90 | −2.25258507 | 0.13892 | 1.18079 | 0.03155 | 2.11018 | 0.9066 |
| FNDC8 \|EGFR \|LSAMP \|SERP4 \|TMEM27 \|CHAC1 \|LRIT3 \|FAM3D \|KIAA1024 \|C12orf77 | −2.26288243 | 0.1824 | 1.18663 | −0.029 | −0.5749 | 0.93408 |
| KRTAP5-8 \|LIPI \|SHROOM3 \|TRPC5 \|ZIC1 \|HRH4 \|TEAD3 \|SERPINA6 \|DSCAML1 \|SMO | −2.27503939 | 0.16805 | 1.04111 | 0.11936 | 0.36738 | 0.92742 |
| C8B \|PSG5 \|CHRNA1 \|MCCD1 \|HPCA \|ANKFN1 \|HIPK4 \|SPATA18 \|FAM228A \|BAG2 | −2.29033687 | 0.15382 | 1.28697 | −0.0065 | 0.01728 | 0.93057 |
| PCDHGA1 \|PCDHB11 \|DOHH \|SLCO1B1 \|PCDHGA12 \|PRM1 \|PCDH11X \|MGARP \|OR9A2 \|ANXA10 | −2.30074244 | 0.16313 | 1.3517 | −0.0093 | −0.6188 | 0.94352 |
| VWCE \|PLA2G1B \|ATP6V1E2 \|KCNJ11 \|SPINK2 \|RAX2 \|SPINK14 \|GLB1L3 \|MMP15 \|THAP10 | −2.31155505 | 0.19726 | 1.18464 | −0.0288 | −0.3926 | 0.92026 |
| SDHAF1 \|MYH15 \|KIF26B \|METTL24 \|XDH \|GHSR \|UGT2B7 \|CHRDL2 \|OR1J4 \|ZNF385B | −2.32333237 | 0.18639 | 1.14493 | 0.00075 | −0.1066 | 0.93291 |
| LCTL \|SFTPA1 \|HPDL \|ZPBP2 \|MAMSTR \|UPK2 \|INS \|COL14A1 \|STEAP1 \|MUCL1 | −2.33643406 | 0.17645 | 1.24008 | 0.06634 | 3.34081 | 0.90266 |
| IGFBP2 \|NTNG1 \|CLDN1 \|CGB1 \|LRFN3 \|SLC17A8 \|SEC14L5 \|C12orf74 \|SLC26A9 \|KCNK17 | −2.34765642 | 0.17563 | 1.15813 | 0.0524 | 2.30939 | 0.91976 |
| PRSS42 \|HSPA12B \|LGR5 \|EMILIN1 \|PSG3 \|MAOB \|FERMT2 \|MRGPRD \|CXADR \|FAM92B | −2.35349116 | 0.17656 | 1.15477 | 0.02868 | −0.6158 | 0.90528 |
| FLG2 \|TCTEX1D1 \|CPSF4L \|PLIN5 \|CNR1 \|RASAL1 \|ZNF503 \|ADAD2 \|ANGPTL2 \|HCN1 | −2.36137264 | 0.17132 | 1.2459 | −0.0082 | 0.46875 | 0.92484 |
| OLFM3 \|RGS4 \|CD34 \|OR2J3 \|MYBPH \|FABP7 \|ALDH1A3 \|CA3 \|LURAP1L \|TCP10L2 | −2.37449653 | 0.16521 | 1.16652 | 0.02799 | 0.59695 | 0.92912 |
| PGF \|OR10A7 \|VSTM5 \|MYL4 \|FAM227A \|SPRR2G \|CRB1 \|C5orf60 \|MORN5 \|OXCT2 | −2.38507436 | 0.1806 | 1.24273 | −0.0389 | 0.77067 | 0.9316 |
| YY2 \|WT1 \|ENTPD8 \|PHACTR3 \|TCHH \|OMD \|PPP1R3E \|SLC6A1 \|MYO18B \|PPP1R14D | −2.38955883 | 0.17313 | 1.18528 | −0.0013 | 0.17739 | 0.92546 |
| CLPS \|EMCN \|UBQLNL \|PRAMEF14 \|GBP7 \|MYL7 \|NAT2 \|C22orf42 \|COLEC12 \|TPH2 | −2.39716986 | 0.12038 | 1.24572 | −0.013 | 1.39201 | 0.93184 |
| SNAP25 \|CIDEC \|QPRT \|OR3A1 \|NPAS1 \|MPP4 \|C2orf71 \|KCNK2 \|IL17C \|SLC7A13 | −2.41399786 | 0.17379 | 1.12141 | 0.0249 | 1.3580L | 0.92147 |
| CCDC96 \|FBXO40 \|FOXD4L3 \|IHH \|C22orf31 \|MARVELD3 \|ANO2 \|FAM171A2 \|MCMDC2 \|RIIAD1 | −2.43237267 | 0.21706 | 0.97631 | 0.08075 | 0.02505 | 0.923 |
| GIP \|SLC2A12 \|TEPP \|PLA2G4F \|CD200R1L \|CAPN11 \|POSTN \|ADH7 \|MAGEA3 \|PRAMEF11 | −2.44358515 | 0.12765 | 1.3491 | −0.0174 | −0.5989 | 0.93354 |
| ZSCAN10 \|NR0B2 \|SYT5 \|OR51D1 \|DEFB125 \|CABP7 \|RPP25 \|DHDH \|ATRNL1 \|EFEMP1 | −2.45323514 | 0.19595 | 1.21593 | 0.00726 | −0.4288 | 0.92112 |
| KRTAP9-3 \|CATSPER4 \|PRM2 \|TBX10 \|SCEL \|PCDHGA3 \|GTF2A1L \|SLC35E4 \|TSSK6 \|LMOD3 | −2.46026335 | 0.17607 | 1.23058 | −0.0262 | 0.33468 | 0.92895 |
| IL11 \|KLK4 \|UMODL1 \|GJB5 \|ADAMTS8 \|KCNC1 \|TPRXL \|C2orf70 \|LCE2A \|GJA1 | −2.46879392 | 0.13811 | 1.29045 | −0.0509 | −0.2496 | 0.91271 |
| RGS16 \|DHH \|KIRREL2 \|KIAA1456 \|CNKSR3 \|MYH8 \|AOX1 \|RXFP1 \|NXNL2 \|DNAH2 | −2.47779401 | 0.20012 | 1.03703 | 0.08149 | 1.12206 | 0.93 |
| FFAR1 \|CXorf66 \|CCBE1 \|EDN3 \|SLCO1B3 \|KCNH7 \|PERGL \|FGA \|OOEP \|PCDHB8 | −2.48599228 | 0.12272 | 1.25221 | −0.0284 | 1.18684 | 0.93679 |
| FOXA1 \|MYOZ3 \|NFATC4 \|MMP13 \|PRICKLE2 \|TSPAN7 \|IL13 \|ELAVL2 \|CNTN6 \|DEFB134 | −2.49451219 | 0.14795 | 1.1873 | 0.02594 | 0.84928 | 0.93677 |
| DDX25 \|CST11 \|MARVELD1 \|CRISPLD1 \|TIP1 \|NEUROD6 \|AFAP1L1 \|TRPM3 \|GTF2IRD1 \|LRRC3C | −2.50440805 | 0.20065 | 0.88377 | 0.10548 | 0.43136 | 0.93048 |
| PAX1 \|SNX7 \|CEND1 \|RND3 \|FAM187B \|ZNF804B \|AKAP3 \|FOXN4 \|OR5B12 \|RIBC2 | −2.5155061 | 0.18666 | 1.1163 | −0.0322 | 1.03397 | 0.92073 |
| OR8B3 \|FRMPD1 \|ICAM5 \|OR51M1 \|PRKD1 \|HEPHL1 \|NGFR \|HECW1 \|NMRK2 \|GPR151 | −2.52455986 | 0.1703 | 1.01347 | 0.05795 | 0.26307 | 0.92902 |
| SLC45A3 \|SMPX \|NPC1L1 \|PRR7 \|E2F8 \|SLC9C1 \|CRMP1 \|MYH6 \|NECAB1 \|KITLG | −2.53098275 | 0.21539 | 1.10911 | 0.01278 | −0.6951 | 0.91658 |
| SLC25A47 \|PLEKHD1 \|CDH4 \|ZC2HC1B \|BDNF \|PRDM9 \|KDR \|DLK1 \|HTR6 \|GRK7 | −2.53717729 | 0.15824 | 1.08299 | 0.05695 | 2.30857 | 0.90962 |
| DCAF4L1 \|AKAP12 \|TMEM37 \|ASCL3 \|CPA1 \|WFDC11 \|IGF1 \|TXNDC8 \|OR51Q1 \|LIPM | −2.55105472 | 0.13595 | 1.23194 | −0.0182 | −0.3637 | 0.94069 |
| OR1J2 \|ZNF8 \|LSM11 \|OTOA \|PRDM7 \|RBAG \|RIMS1 \|FMO6P \|SCHIP1 \|SOX30 | −2.56390204 | 0.19548 | 1.07663 | 0.06223 | −0.3465 | 0.93683 |
| C9orf66 \|TMEM200B \|ZNF214 \|C12orf71 \|GALNTL5 \|SLC10A5 \|SPINT3 \|LCNL1 \|ACOT12 \|TMEM249 | −2.57148608 | 0.18401 | 1.24646 | −0.0213 | −0.6655 | 0.92605 |
| MYH2 \|FGF22 \|OR2V2 \|NUDT11 \|GCM1 \|OR1E2 \|KDM4D \|SEMAGD \|ESYT3 \|C17orf77 | −2.58011523 | 0.18896 | 1.15399 | 0.03072 | 3.06395 | 0.93743 |
| APOB \|KRT15 \|CPNE4 \|ZNF771 \|PRR9 \|PTGDR2 \|STOX2 \|FOXH1 \|APCS \|ACADL | −2.59060174 | 0.19685 | 1.36458 | −0.0074 | 0.48127 | 0.92305 |
| ZAR1L \|SLC6A5 \|MRO \|CCDC168 \|DNAH9 \|KCNJ14 \|ORSAC2 \|PRG2 \|TRIM39-RPP21 \|SYNPO2L | −2.60103346 | 0.17778 | 1.24858 | 0.03151 | 0.35815 | 0.93846 |
| TSPAN6 \|CYP26A1 \|DEFB119 \|CSNK1A1L \|PRSS48 \|TAS2R42 \|KLK14 \|KIAA1161 \|TGM6 \|C11orf96 | −2.61375722 | 0.14918 | 1.20624 | −0.0091 | −0.033 | 0.92366 |
| SLC35F4 \|LGALS16 \|LINGO2 \|GSC \|MAGEC1 \|CCDC54 \|HOXA5 \|EFHD1 \|CAMK2N1 \|EFCAB3 | −2.62558288 | 0.14532 | 1.20911 | −0.0288 | −0.5669 | 0.93625 |
| KCNK1 \|NT5C1B-RDH14 \|KRT14 \|IL17A \|ALPP \|COL6A6 \|EVPLL \|IZUMO1 \|BEX1 \|SGCG | −2.63413853 | 0.15433 | 1.19354 | 0.05135 | 0.32512 | 0.9279 |
| MYOZ2 \|CLCA4 \|SERTAD4 \|ARID3C \|POU1F1 \|SPARCL1 \|PI15 \|PTH \|RBFOX3 \|UGT1A7 | −2.64191956 | 0.13154 | 1.40084 | −0.0833 | −0.1096 | 0.94375 |
| REG1B \|FMO1 \|PDILT \|BRDT \|NUDT8 \|SCG2 \|TMPRSS11BNL \|PAX2 \|ABCG2 \|MYH4 | −2.65431357 | 0.14706 | 1.22992 | 0.04375 | 0.31524 | 0.93849 |
| DAB2IP \|EPS8L3 \|SDR16C5 \|ECE2 \|MT3 \|SPC24 \|OR5K4 \|SPO11 \|CELA3B \|TEAD1 | −2.66440488 | 0.176 | 1.29922 | −0.0026 | −0.368 | 0.92253 |
| RAB6C \|TAGLN3 \|KLK2 \|SYT2 \|TTC9B \|NBPF4 \|PKHD1 \|CLDN19 \|GPR182 \|IMPG1 | −2.67506728 | 0.15839 | 1.23562 | 0.02425 | 0.75664 | 0.92064 |
| HPSE2 \|RAG2 \|RHOXF2B \|CGB7 \|RAB42 \|LRRIQ3 \|SLCO1B7 \|RSPH6A \|NLRP14 \|SERPINA3 | −2.68442624 | 0.19077 | 0.95706 | 0.07549 | −0.0474 | 0.92828 |
| WNK3 \|UGT1A9 \|PLEKHA6 \|MAB21L1 \|AKR1B10 \|PTGES \|SCN11A \|SEMG1 \|FUT1 \|FAM109B | −2.69694005 | 0.17535 | 1.29502 | 0.01215 | −0.443 | 0.92509 |
| ADAMTS2 \|FAM83E \|GRK1 \|IL19 \|TACC2 \|CCDC70 \|CDCP1 \|COL22A1 \|MIOX \|LYPD5 | −2.70802259 | 0.14234 | 1.14332 | 0.017 | −0.8806 | 0.91116 |
| CPN1 \|FAM183A \|PI4K2B \|GABRB3 \|FAM171B \|GPR150 \|ASXL3 \|OR52B2 \|C12orf50 \|TNFRSF19 | −2.71986153 | 0.19407 | 1.03711 | 0.04592 | −0.4552 | 0.93369 |
| PLEKHS1 \|KIAA0319 \|NXPH3 \|CSMD3 \|WISP1 \|SLC38A8 \|CALML6 \|CMYA5 \|ACOT6 \|RETNLB | −2.73041982 | 0.16669 | 1.14831 | 0.045 | 1.82375 | 0.92174 |
| CRYBA4 \|NME5 \|DUXA \|PPP1R27 \|CHRNB4 \|PKP1 \|C9orf116 \|COL15A1 \|ITGBL1 \|ODF3L1 | −2.739073 | 0.16957 | 1.22498 | 0.03505 | 0.75803 | 0.92176 |
| CASKIN2 \|MFAP2 \|SORCS2 \|KCNE4 \|ABCC12 \|FOXD1 \|PSPN \|MYT1 \|ACSS3 \|NPAP1 | −2.75060081 | 0.16951 | 1.22359 | 0.03658 | 0.49916 | 0.91478 |
| INSC \|ZNF728 \|PCDHGC4 \|POTEM \|PRRT4 \|DCX \|CDH10 \|KCNT1 \|GSDMA \|AQP8 | −2.76119896 | 0.15747 | 1.12899 | 0.02165 | −0.1722 | 0.92713 |
| SERPINB13 \|NELL1 \|ABCC11 \|C4orf47 \|TGFB2 \|KRT26 \|GLP2R \|OR2A2 \|SLC24A5 \|PDCL2 | −2.77465653 | 0.12848 | 1.25073 | −0.0488 | −0.1372 | 0.93909 |
| IL12B \|LELP1 \|TPGS1 \|PDGFRL \|SULT1C2 \|MPV17L \|HES2 \|P2RX3 \|PTPN14 \|RGS11 | −2.78420911 | 0.1859 | 1.27746 | −0.0564 | −0.0694 | 0.9193 |
| LMAN1L \|SGCZ \|SOX11 \|UMOD \|OR1L8 \|C14orf132 \|HIST1H1T \|SLC25A52 \|ZNF534 \|LARP6 | −2.7939936 | 0.14774 | 1.17537 | 0.05662 | −0.2248 | 0.93212 |
| ZNF474 \|BAAT \|KCNT2 \|CYP2W1 \|CPB1 \|ELANE \|FAM57B \|ASB12 \|TMPRSS11A \|TRIM54 | −2.80617474 | 0.1664 | 1.22902 | 0.01819 | 0.5939 | 0.92576 |
| AMZ1 \|PSG9 \|T \|TMEM240 \|ASTN1 \|C1orf116 \|GABARAPL3 \|MAP6 \|LRRC32 \|ROBO4 | −2.82068372 | 0.16817 | 1.22511 | −0.0503 | 1.06394 | 0.91172 |
| GLRA3 \|PRR5-ARHGAP8 \|C19orf73 \|SLC24A2 \|CADM3 \|TPO \|BVES \|FAM84B \|TYRP1 \|C10orf99 | −2.83087469 | 0.19219 | 1.11496 | 0.03764 | −0.1984 | 0.93006 |
| FRAS1 \|SLC52A3 \|LALBA \|ARHGEF15 \|RTBDN \|PATE1 \|TRIML2 \|OR2T11 \|DGKB \|PALMD | −2.84046664 | 0.14298 | 1.25134 | −0.0383 | 1.10142 | 0.93327 |
| DAAM2 \|CYP4X1 \|GRIN2D \|TRPM5 \|OPN5 \|LGALS7 \|BMX \|MAGEA11 \|SCUBE1 \|UGT2B11 | −2.85935107 | 0.1483 | 1.27018 | −0.0313 | −0.3175 | 0.91801 |
| ZNRF3 \|GGT6 \|KLB \|KCNIP1 \|BOK \|MYPOP \|SLC2A4 \|CDH2 \|CYP7B1 \|NTSR1 | −2.87298429 | 0.22109 | 1.13632 | 0.01949 | −0.8009 | 0.91147 |
| OR52L1 \|PSG6 \|JAKMIP3 \|OR1Q1 \|OR10G4 \|KRT12 \|ZBTB46 \|CLEC2A \|SRPX \|OXGR1 | −2.88378224 | 0.12032 | 1.20175 | −0.0278 | 0.90691 | 0.93272 |
| LYPD3 \|CXCL14 \|CMBL \|SOWAHC \|CSDC2 \|SLC6A9 \|TIAF1 \|IDI2 \|OR6C74 \|TBL1Y | −2.89433701 | 0.17565 | 1.23538 | −0.011 | 1.75492 | 0.92006 |
| PCDH18 \|ELAVL4 \|OR2T33 \|HNRNPCL1 \|DPYSL3 \|MYH7 \|TICRR \|C8orf22 \|GALR2 \|PTPRR | −2.90234417 | 0.15475 | 1.0371 | 0.04308 | 0.11143 | 0.93897 |
| DCHS2 \|PRAME \|PEX5L \|SLC6A3 \|TINAGL1 \|SERPINA9 \|PAX3 \|GRIK3 \|TMC7 \|LRRN4 | −2.91379507 | 0.14077 | 1.26314 | 0.02545 | 1.39922 | 0.91353 |
| SYT4 \|ZIC2 \|IRS4 \|FGL1 \|GJA5 \|CTRB2 \|MDH1B \|SGK2 \|WDR87 \|KRTAP5-6 | −2.92588587 | 0.18786 | 1.23783 | 0.04304 | −0.2084 | 0.91921 |
| SERPINB7 \|VTCN1 \|GALP \|HNF1B \|MUC22 \|AQP12B \|HS3ST1 \|RARRES2 \|TPRX1 \|AJUBA | −2.93299841 | 0.14342 | 1.29577 | 0.00834 | −0.1419 | 0.90521 |
| OR4Q3 \|TTC29 \|NXPH4 \|PSCA \|BHLHB9 \|GABRB2 \|EEF1A2 \|MAT1A \|UBEZU \|ARTN | −2.94141146 | 0.17175 | 1.09261 | 0.07516 | −0.3187 | 0.92347 |
| FMN2 \|LRP4 \|CHRNA6 \|SLC19A3 \|HOXB13 \|COL12A1 \|GSG1L \|OR51V1 \|PLD5 \|G6PC2 | −2.94807232 | 0.15007 | 1.02466 | 0.08145 | 1.33969 | 0.92216 |
| AGXT2 \|TSPAN8 \|KCNIP3 \|PROKR2 \|ANKRD34B \|RELN \|STRA6 \|OR5C1 \|PCDHB6 \|OR2L13 | −2.95481345 | 0.1331 | 1.16316 | −0.0042 | 0.45986 | 0.92224 |
| UGT2B4 \|N4BP3 \|TTR \|TMC5 \|KLK15 \|CASR \|FBLIM1 \|ARL9 \|CACNG8 \|CYP27C1 | −2.9655753L | 0.14394 | 1.17145 | 0.03529 | 1.06135 | 0.92453 |
| RGMA \|IL34 \|KRTAP2-1 \|MLPH \|PIP \|DCSTAMP \|SFTPC \|IGFL1 \|RERG \|TMEM238 | −2.9749317 | 0.15065 | 1.20574 | 0.02112 | 0.23123 | 0.91994 |
| CXCL9 \|ADAMTSL1 \|CDO1 \|HOXA6 \|CHRM3 \|ZIC4 \|PCDHB14 \|OR10G3 \|LUZP2 \|SPEF1 | −2.98168249 | 0.16479 | 1.20732 | −0.0243 | −0.1751 | 0.93846 |
| PLA1A \|GC \|TMC2 \|MEIS3 \|WFDC5 \|CGA \|GPR137C \|SERP5 \|GPR153 \|GPR1 | −2.99118301 | 0.1553 | 1.28628 | −0.0446 | 0.70483 | 0.92321 |
| SMAD9 \|C15orf53 \|GJB3 \|CCER1 \|GLOD5 \|EXOC3L2 \|SLC25A2 \|PALM \|UNC13A \|SLPI | −3.00735411 | 0.19224 | 1.16094 | −0.0266 | 0.19289 | 0.91551 |
| GABRA2 \|TCL1B \|MT1H \|SLC7A3 \|RFX8 \|MMP26 \|GNA14 \|ZNF454 \|TMEM202 \|CARD14 | −3.02125923 | 0.15137 | 1.0485 | 0.0436 | −0.1848 | 0.93166 |
| SIM2 \|HSD17B1 \|RPE65 \|OR10P1 \|FNDC7 \|SLC25A18 \|GFRA1 \|RAI2 \|ABCB5 \|SAMD7 | −3.03282161 | 0.14449 | 1.26748 | −0.0132 | −0.7922 | 0.94397 |
| ST8SIA2 \|SRRM4 \|BMP7 \|LDLRAD1 \|CCL1 \|DUSP15 \|MEX3D \|PITX2 \|CPLX3 \|DMRT1 | −3.04140305 | 0.15361 | 1.06017 | 0.06927 | 2.20095 | 0.90215 |
| HRC \|INHBA \|MRGPRF \|SSX3 \|BCL2L10 \|GPR63 \|CPEB1 \|OR4F5 \|ESRRB \|NXPE2 | −3.05170247 | 0.16195 | 1.16768 | 0.00767 | 3.33947 | 0.92284 |
| SIGLEC5 \|LIPK \|PSMA8 \|KCNU1 \|C10orf67 \|KHDC1L \|KCNE2 \|KRTAP21-1 \|BCAN \|PLA2G2D | −3.06198968 | 0.15278 | 1.23842 | 0.0243 | −0.3531 | 0.93913 |
| KCNN1 \|NIN3 \|DLL3 \|AQP2 \|CNTN5 \|GRIN2A \|ZFP41 \|ATP1A4 \|ADAMTSL2 \|TMEM198 | −3.07094637 | 0.21395 | 1.14779 | −0.0115 | 0.04711 | 0.90023 |
| HIF3A \|GKN1 \|ASPRV1 \|ISL2 \|KRTAP10-11 \|NPBS1 \|SLITRK3 \|GJC1 \|OR11H4 \|TMEM132E | −3.08492274 | 0.15845 | 1.23827 | 0.00734 | −0.2858 | 0.91996 |
| ARMS2 \|IZUMO2 \|SLC9A4 \|NRK \|ARSH \|LCE5A \|PDHA2 \|SYNGR3 \|CIB4 \|SHISA3 | −3.09298221 | 0.13469 | 1.26213 | −0.0492 | 1.46591 | 0.92406 |
| ZFP92 \|GRM8 \|CSAG1 \|GIPC2 \|KCNH4 \|SEZ6L2 \|CSRNP3 \|SERPINA7 \|MUC21 \|UGT1A10 | −3.10155552 | 0.21039 | 1.12153 | 0.02801 | −0.6563 | 0.92749 |
| KCNJ5 \|FAM13C \|ZNF645 \|MT1A \|PPP1R3D \|LCE3D \|HRH3 \|FAM25A \|PCDHB7 \|PITX1 | −3.1093459 | 0.1763 | 1.00253 | 0.09357 | −0.232 | 0.91476 |
| CACNA1E \|BMP4 \|MATN3 \|GPR45 \|MAGEA8 \|VSX1 \|C7orf57 \|IQCF3 \|SMYD1 \|DYDC2 | −3.12536666 | 0.16183 | 1.1944 | 0.04617 | 2.71109 | 0.93214 |
| GLUD2 \|SBK2 \|HTR3B \|CYP39A1 \|TMEM105 \|IL4 \|ARHGAP20 \|TSGA13 \|USH2A \|OLAH | −3.13399457 | 0.16246 | 1.24984 | −0.0164 | −0.4769 | 0.93232 |
| FAM71F1 \|CAMK2A \|SCGB2B2 \|ANO4 \|ENPP7 \|COBL \|TMEM98 \|CA5A \|NKAIN2 \|BTC | −3.1417584 | 0.17199 | 1.23144 | −0.0022 | 1.12474 | 0.91929 |
| GALNT5 \|TLL1 \|DUOXA2 \|PTPRF \|DZIP1 \|CLDN9 \|TNP2 \|IQCF2 \|PSKH2 \|NKX6-2 | −3.15048836 | 0.16106 | 1.21476 | −0.0047 | 1.9312 | 0.92552 |
| DRD3 \|CCDC42 \|NRXN3 \|DLX4 \|ABCG5 \|CPLX4 \|C2CD4D \|NYNRIN \|BTNL3 \|AZGP1 | −3.16344877 | 0.17032 | 1.22827 | 0.02598 | 3.75724 | 0.9246 |
| FAM222A \|RNASE12 \|GDA \|PRG3 \|WFIKKN1 \|C17orf98 \|BOC \|ISX \|TFF3 \|MYH1 | −3.17154028 | 0.15668 | 1.35875 | 0.00368 | 1.41902 | 0.9213 |
| GSTA2 \|PTF1A \|FABP6 \|DUPD1 \|F2RL3 \|SHC4 \|NCAM2 \|HSPB8 \|ZFHX4 \|CRYGB | −3.18576374 | 0.18755 | 1.26541 | 0.00165 | 2.0195 | 0.92411 |
| ZNF676 \|TRIM15 \|XCR1 \|TCEAL6 \|IFNA1 \|PRTG \|LY6K \|RGSL1 \|KRTAP10-8 \|C6orf118 | −3.20694758 | 0.13198 | 1.28423 | 0.00024 | −0.1442 | 0.93007 |
| LEFTY1 \|MYOC \|TMEM17 \|ALPK3 \|PTGIS \|TMEM89 \|TRPC3 \|PCDHA7 \|SLC52A1 \|GPC4 | −3.21859771 | 0.18067 | 1.23168 | 0.00645 | 0.64023 | 0.91384 |
| XAGE5 \|KIF4B \|ZBTB8A \|NRN1 \|FRG2C \|KISS1R \|DCD \|GCGR \|FAM19A4 \|AFM | −3.22986198 | 0.17767 | 1.08458 | 0.08077 | −0.3158 | 0.92946 |
| STXBP5L \|DSC3 \|VWA2 \|OR14J1 \|BSPH1 \|CCDC27 \|IGSF9 \|FAM169B \|C17orf47 \|CLDN16 | −3.23806355 | 0.1379 | 1.38841 | −0.0118 | 1.04186 | 0.92778 |
| ERVV-2 \|CAPSL \|HSD3B1 \|TMEM119 \|F10 \|IQUB \|ADAM7 \|MINR1B \|KRT75 \|SUCNR1 | −3.2448235 | 0.13635 | 1.27325 | −0.04 | 0.08433 | 0.92877 |
| LPIN3 \|ADH1B \|MATN1 \|MUC17 \|AGR3 \|IFNA2 \|TUBA3E \|ANGPT4 \|PRSS58 \|TMPRSS11F | −3.25286951 | 0.15581 | 1.27313 | −0.0532 | −0.3806 | 0.9266 |
| SPINK13 \|C14orf39 \|LHFPL3 \|MAG \|SPANXN2 \|RGN \|ERVV-1 \|SMOC2 \|SLC28A1 \|CPXM1 | −3.26009799 | 0.12492 | 1.18059 | 0.0309 | 0.08426 | 0.92212 |
| CARD18 \|SSX5 \|HTR2A \|TNFAIP8L1 \|KLHDC7B \|GAS2L2 \|NEUROD1 \|KRT83 \|LRFN5 \|C6orf58 | −3.27325104 | 0.17099 | 1.2855 | 0.01239 | 0.73096 | 0.93135 |
| GRM5 \|POM121L2 \|JPH1 \|FGB \|HOXA7 \|OR56A4 \|GPX5 \|PCDH11Y \|DRD5 \|KPRP | −3.29579017 | 0.1437 | 1.10358 | 0.01718 | −0.3114 | 0.93491 |
| NPFF \|PGLYRP3 \|SEC14L6 \|PANX3 \|EDIL3 \|DPPA5 \|KLK11 \|SYT14 \|APOD \|ZPLD1 | −3.31053718 | 0.11838 | 1.21581 | 0.00891 | −0.1256 | 0.92693 |
| SLC17A1 \|PTCHD1 \|PASD1 \|CA9 \|SOCS1 \|FNDC4 \|CACNA1S \|TSKU \|CGNL1 \|EPHA5 | −3.3194159 | 0.17465 | 1.14106 | 0.05393 | 1.66764 | 0.91755 |
| FABP2 \|PAX4 \|EBI3 \|HRCT1 \|NOS1AP \|ISM2 \|OR5P3 \|ENO4 \|TNS4 \|TRIM10 | −3.32708754 | 0.18761 | 1.16998 | −0.0091 | 0.98548 | 0.91666 |
| BTN1A1 \|EFCAB9 \|WFIKKN2 \|BEST3 \|PROX2 \|SLC22A2 \|KIAA1549 \|PCDHB15 \|GABRA3 \|MESP1 | −3.3334389 | 0.18586 | 1.23983 | −0.0035 | 0.19436 | 0.92968 |
| UROCH1\|ARHGEF17 \|CDH11 \|AGR2 \|GRIK2 \|COX412 \|LYZL1 \|BIRC7 \|DCAF8L2 \|C11orf88 | −3.34579998 | 0.16589 | 1.28565 | −0.0162 | 2.94804 | 0.91933 |
| SULT2A1 \|EBLN1 \|SCGB1D4 \|CCDC144NL \|OR13A1 \|KIF18B \|PRSS54 \|RJMBP2 \|SCGB1A1 \|SLC7A4 | −3.35992476 | 0.13531 | 1.14842 | 0.00488 | 0.4374 | 0.93327 |
| HOXD10 \|TMEM155 \|COL23A1 \|RDH8 \|SFTA3 \|INPP5J \|PRAMEF13 \|SCARA5 \|FHDC1 \|SLC9C2 | −3.37421408 | 0.16323 | 1.16088 | 0.00132 | 5.63162 | 0.92247 |
| OR51G1 \|B3GAT2 \|ADAMTS12 \|GNG13 \|GREM1 \|FLRT1 \|ARSJ \|STH \|SLC22A9 \|LHX1 | −3.38643497 | 0.12295 | 1.21392 | 0.0342 | −0.6123 | 0.91777 |
| CSF3 \|GGTLC1 \|CELF3 \|C7orf72 \|SYNDIG1L \|IFITM10 \|NKX3-1 \|ATP10B \|LYPD6B \|ITIN6 | −3.39948252 | 0.16194 | 1.05033 | 0.03622 | 0.11958 | 0.90951 |
| OR2A14 \|DEFA5 \|SLC35G1 \|NR1H4 \|RANBP3L \|HSD3B2 \|BCL2L14 \|CCL25 \|CBLN1 \|GLYATL2 | −3.4111227 | 0.16309 | 1.28028 | −0.0503 | −0.7856 | 0.93899 |
| OR51L1 \|OR52N2 \|CCDC129 \|WFDC1 \|NWD1 \|ENPEP \|UNC80 \|SCGB1D1 \|SHPK \|LRRC72 | −3.42436997 | 0.15788 | 1.23795 | 0.02085 | −0.3942 | 0.93472 |
| HRASLS2 \|AHSG \|MSANTD1 \|BSN \|ADRA1A \|MYOZ1 \|F9 \|CAPN13 \|KNCN \|RSPO3 | −3.43828363 | 0.15277 | 1.14174 | 0.04251 | 1.36634 | 0.91674 |
| GAP43 \|ZNF114 \|PYGO1 \|RCAN2 \|NDST3 \|KRT19 \|FERMT1 \|CHST8 \|MYL3 \|OR13G1 | −3.45122185 | 0.16101 | 1.27879 | 0.01586 | 0.01328 | 0.92452 |
| MAGEC3 \|OR2A25 \|TEX33 \|LGI2 \|PTPRH \|SCNN1G \|KRTAP19-5 \|ARHGAP39 \|CRISP1 \|PDXP | −3.4664563 | 0.15505 | 1.25018 | 0.02079 | −0.4035 | 0.93271 |
| SPRN \|OLIG2 \|ACBD7 \|NOS2 \|FREM2 \|XRCC2 \|TNFSF9 \|C17orf105 \|CDC20B \|YJEFN3 | −3.47671445 | 0.22501 | 0.98475 | 0.05157 | −0.3594 | 0.91519 |
| CLCNKB \|SLC16A14 \|PAEP \|SYT10 \|ATP2B2 \|SCN7A \|KRT6C \|SH2D6 \|FGF6 \|KRT31 | −3.48888168 | 0.12929 | 1.21056 | −0.0444 | 2.33351 | 0.91391 |
| GLI2 \|DYDC1 \|SPANXN5 \|SHISA8 \|PREX2 \|TREH \|THSD4 \|NXPH1 \|DBNDD1 \|SLC30A8 | −3.4994955 | 0.14347 | 1.3364 | −0.0601 | 0.83337 | 0.92543 |
| TMEFF1 \|VPREB1 \|DEFA6 \|KCND2 \|MDGA2 \|SH3GL3 \|SP8 \|CALCB \|VWA5B2 \|NEU4 | −3.51163618 | 0.15769 | 1.28314 | −0.0223 | 1.22225 | 0.91668 |
| GPR161 \|OR2T10 \|GABRA4 \|TRIM6-TRIM34 \|FGD5 \|MYBPHL \|OR8D2 \|CACNA1G \|SLC7A14 \|RAET1L | −3.52156259 | 0.17278 | 1.18671 | −0.0081 | 3.10373 | 0.92642 |
| OR4D6 \|B4GALNT2 \|KRTAP2-4 \|GBX1 \|C9orf57 \|FRK \|GUCY1A2 \|CBLC \|SPATA2L \|NANOG | −3.53343559 | 0.17838 | 1.17259 | −0.0549 | 0.08491 | 0.93 |
| CASP14 \|GADL1 \|CSN2 \|ABCB11 \|MSTN \|RASIP1 \|HS3ST4 \|ADAD1 \|CLCA1 \|DEFB1 | −3.54655554 | 0.14719 | 1.28709 | 0.01115 | 2.21075 | 0.93628 |
| CPB2 \|USP26 \|CELF5 \|PARD6G \|AMY1A \|CLDN10 \|RADIL \|MFRP \|CHST13 \|ENOX1 | −3.55253945 | 0.20914 | 0.99081 | 0.08828 | −0.8068 | 0.91886 |
| DUOXA1 \|TPBG \|OR4K1 \|ZPBP \|FILIP1 \|DSG4 \|KCNJ10 \|OR5H14 \|CCDC74A \|OR4A16 | −3.56429627 | 0.16166 | 1.12626 | 0.02904 | 2.50441 | 0.94278 |
| SPRR2D \|TMEM95 \|PEK3C2G \|DPPA3 \|ZIM3 \|OR7C1 \|ADAM33 \|GJD2 \|EFCAB1 \|DNAAF3 | −3.57747778 | 0.15898 | 1.19704 | 0.00684 | −0.3529 | 0.93527 |
| SLC16A8 \|FGFR4 \|ISY1-RAB43 \|SCNN1B \|DLX1 \|SELE \|CEACAM5 \|C10orf95 \|FBXW12 \|HAP1 | −3.58478178 | 0.20687 | 1.09785 | 0.0226 | −0.4612 | 0.91273 |
| TTPA \|NR2F2 \|SERTM1 \|GDF5 \|ADM2 \|OR4D5 \|CCDC60 \|C15orf56 \|UBQLN3 \|PCDHA6 | −3.59263705 | 0.18353 | 1.05136 | 0.02174 | −0.5736 | 0.92724 |
| STK32A \|DMP1 \|OR283 \|NXF2 \|GSTA5 \|TLR9 \|BPIFB4 \|TMIGD1 \|SCN4A \|AMBN | −3.60989906 | 0.16482 | 1.36042 | 0.00031 | 0.42089 | 0.92755 |
| PKNOX2 \|GOLT1A \|TMEM213 \|C22orf24 \|OR4M1 \|ANO1 \|AQP5 \|HHATL \|TLCD2 \|GPRC5A | −3.62372711 | 0.18046 | 1.03831 | 0.1037 | 0.04368 | 0.9101 |
| ALOXE3 \|KCNN2 \|TAS2R38 \|CDK15 \|DKKL1 \|RBP4 \|CEACAM6 \|ANKRD45 \|TMEM221 \|LAMC2 | −3.63974671 | 0.18859 | 1.23412 | −0.0489 | −0.8721 | 0.93272 |
| PANX2 \|OTX2 \|AARD \|KRTAP6-1 \|RAET1E \|GLYAT \|KRTAP5-9 \|ADAMTS14 \|CACNG2 \|CDH7 | −3.65016765 | 0.1423 | 1.17511 | 0.04454 | 2.42912 | 0.92823 |
| TNFSF15 \|CLRN2 \|EXOC3L4 \|ZBED6 \|B3GALT6 \|FAM155B \|MAGEB10 \|UNCSB \|FOXA2 \|G6PC | −3.66321797 | 0.19567 | 0.9977 | 0.03167 | 0.93448 | 0.92692 |
| OR7D2 \|SPATA16 \|TMEM246 \|KCNC2 \|ZBTB7C \|CLRN3 \|PCDHB13 \|ASB4 \|PKLR \|AGXT | −3.67233768 | 0.1489 | 1.23729 | 0.03517 | 0.22522 | 0.93719 |
| KCNJ8 \|HR \|TEX26 \|GJA10 \|BEND3 \|MSX1 \|FOXE1 \|NRG2 \|CSPG5 \|MAMDC2 | −3.68323205 | 0.17552 | 1.16562 | 0.06898 | −0.5746 | 0.91948 |
| NRAP \|DPEP1 \|FAM71A \|IQGAP3 \|AWAT2 \|CRISP2 \|ATP1B4 \|RNF148 \|C4BPA \|UNC79 | −3.69376547 | 0.16739 | 1.24447 | 0.01949 | −0.921 | 0.9365 |
| SPINT4 \|TIGD5 \|PPP1R14C \|SFRP2 \|NPFER2 \|KRTAP10-9 \|DNER \|ZCCHC13 \|HSPB7 \|SLCO1A2 | −3.70079384 | 0.15039 | 1.22695 | 0.02613 | −0.1968 | 0.92101 |
| TDGF1 \|EDDM3A \|LRRC2 \|CLCA2 \|GPR173 \|CXXC4 \|FAM107A \|EIF4E1B \|MEX3B \|MYCN | −3.70553945 | 0.16142 | 1.20873 | −0.0612 | −0.2238 | 0.92683 |
| NYAP1 \|MLNR \|PNLIP \|OTX1 \|CRYBB2 \|CLDN2 \|TGM3 \|TMEM26 \|CERS1 \|TBX4 | −3.7162713 | 0.1775 | 1.1181 | −0.0193 | 0.73116 | 0.92046 |
| GGT5 \|OR52A1 \|GPR156 \|SLC6A17 \|KRT222 \|C11orf70 \|PGBD5 \|EMP2 \|DMGDH \|ARX | −3.73049994 | 0.15019 | 1.13031 | 0.05368 | 3.87633 | 0.92841 |
| IGF2 \|VWA5B1 \|DMRTC2 \|C6orf132 \|B4GALNT1 \|MOBP \|RBMXL2 \|APOBEC1 \|C1orf141 \|MYH13 | −3.74024579 | 0.11986 | 1.2875 | −0.0407 | −0.844 | 0.93508 |
| SHC3 \|FOXE3 \|FAM83F \|CEACAM7 \|PTX4 \|PKD2L1 \|FBXO47 \|CYP26B1 \|DRP2 \|IGSF3 | −3.75563511 | 0.20249 | 1.16612 | −0.0083 | −0.431 | 0.90617 |
| GPR37L1 \|PLA2G10 \|FAM205A \|KIF6 \|SPAG6 \|MMP20 \|RGL3 \|A4GALT \|CALHM3 \|EDNRB | −3.77185752 | 0.17839 | 1.1599 | 0.03323 | −0.5288 | 0.92958 |
| CAMK2B \|NR2E1 \|SLC17A2 \|GLIS2 \|S100A3 \|KRTAP10-7 \|CYP1A2 \|NOX5 \|HMGCS2 \|MAP3K15 | −3.78298227 | 0.17566 | 1.30493 | −0.0007 | 1.04815 | 0.9177 |
| ENTHD1 \|SPACA3 \|CYP4Z1 \|PALM2-AKAP2 \|CHST5 \|PTN \|AKAP4 \|ADAM18 \|SYCE1 \|MSLN | −3.80181551 | 0.1.181 | 1.34381 | −0.0602 | 0.36283 | 0.92401 |
| HNF4A \|OR6C2 \|RASL10A \|CPA6 \|GLRA2 \|UPP2 \|SLC16A12 \|HPGDS \|HOXB6 \|ZSCAN5B | −3.81517724 | 0.17221 | 1.05466 | 0.10427 | −0.5752 | 0.93491 |
| LURAP1 \|NLRP4 \|NLRP10 \|KCNH5 \|CA10 \|TMEM130 \|RHOJ \|C10orf53 \|OR2T27 \|COL4A2 | −3.83419368 | 0.16887 | 1.12482 | −0.0298 | 0.2918 | 0.91644 |
| CHODL \|OR51B4 \|EPHA10 \|MC5R \|GABRA5 \|TRIM29 \|C10orf107 \|NPSR1 \|ADRA2A \|CARTPT | −3.84025945 | 0.13735 | 1.28991 | 0.00939 | 0.52123 | 0.93433 |
| INSRR \|HOXC10 \|SCN10A \|TDO2 \|IGFBP5 \|GDNF \|CTU1 \|FZD8 \|PSMB11 \|TBX15 | −3.85207378 | 0.1883 | 1.19932 | −0.0334 | 0.03829 | 0.91914 |
| PGK2 \|LAMB4 \|PAX9 \|C12orf56 \|FZD5 \|SEMA6B \|HAO2 \|PROX2A \|ADORA1 \|SLC22A31 | −3.86338194 | 0.19682 | 1.18737 | −0.031 | 1.35613 | 0.92918 |
| ELSPBP1 \|GPX6 \|IL1RL2 \|OR2G3 \|BEST4 \|SOX2 \|MALL \|TTBK1 \|PCSK9 \|PROX1 | −3.87510175 | 0.16926 | 1.17468 | −0.0107 | 0.6261 | 0.92325 |
| ZYG11A \|ELAVL3 \|CLEC19A \|RAB17 \|C3orf22 \|TAF1L \|SLC22A25 \|OR51B5 \|UNC5D \|ODAM | −3.88730213 | 0.13741 | 1.03779 | 0.06882 | −0.3164 | 0.93532 |
| HTR3C \|HSPB6 \|OR4E2 \|KLF15 \|OR2A5 \|CHRM2 \|ARHGEF37 \|SVEP1 \|CD164L2 \|IL33 | −3.89935822 | 0.18491 | 1.19608 | 0.00955 | 0.88692 | 0.93428 |
| RAB9B \|IL37 \|OR52R1 \|TEX15 \|INHBE \|FOXJ1 \|BHMT \|DCLK1 \|NTF3 \|SLC10A4 | −3.90991208 | 0.18186 | 1.07899 | 0.06287 | 0.01217 | 0.92654 |
| GUCA2A \|OR2G2 \|ITIH5 \|DRGX \|KPNA7 \|MPPED1 \|C2orf50 \|OR2G6 \|RALYL \|RAD21L1 | −3.91949986 | 0.15719 | 1.11475 | −0.003 | −0.3444 | 0.92487 |
| POPDC3 \|TDRD10 \|LIN28A \|PTGFR \|LECT2 \|TKTL2 \|CDH18 \|SHANK3 \|BCHE \|LCE1E | −3.92872553 | 0.16248 | 1.24531 | −0.021 | 0.58727 | 0.94216 |
| DAZ2 \|SSTR5 \|STRA8 \|PAGE3 \|PI3 \|LYPD1 \|MFAP5 \|CYP8B1 \|GDF6 \|GPR61 | −3.94569841 | 0.15161 | 1.17634 | 0.04878 | −0.2988 | 0.91932 |
| OR10A2 \|FAM83A \|IGDCC4 \|NRG3 \|SDR9C7 \|SHC2 \|KCNS3 \|CLIC6 \|MMP10 \|PNLIPRP1 | −3.95395022 | 0.14565 | 1.19935 | 0.02706 | 0.72181 | 0.92549 |
| PNMA5 \|NYAP2 \|YAP1 \|THEG \|PCSK2 \|GRB7 \|LIPJ \|ZCCHC12 \|DHRS7C \|OR964 | −3.96450883 | 0.13766 | 1.18725 | −0.0203 | −0.5092 | 0.93131 |
| ADRB1 \|STMN2 \|SLC5A8 \|NAT14 \|SERPINA5 \|ROS1 \|DHRS2 \|KLF14 \|KRTAP12-1 \|ARGFX | −3.97562934 | 0.1803 | 1.28521 | 0.0353 | −0.0909 | 0.93637 |
| TEAD4 \|ZP4 \|IFNE \|ADAMTS7 \|SPANXN3 \|CNGA3 \|IL9 \|PRSS57 \|TAL2 \|TRIM50 | −3.98915799 | 0.17306 | 1.18302 | −0.022 | 2.75517 | 0.92961 |
| GPR85 \|RGS13 \|CYP26C1 \|CTCFL \|KRTAP10-4 \|ZDHHC15 \|SPTBN4 \|PTGER1 \|NDP \|IL13RA2 | −4.00626161 | 0.20736 | 1.22482 | −0.0918 | −0.4946 | 0.92981 |
| PEG3 \|BTBD16 \|STAC2 \|SERPINA11 \|SLC25A31 \|DNAJC12 \|GSTT2 \|SCTR \|CTGF \|MS4A12 | −4.01790277 | 0.15212 | 1.23816 | 0.03684 | 2.2885 | 0.93231 |
| CILP \|GDF1 \|RHOD \|KLHL31 \|GPR83 \|SLC25A21 \|ZP2 \|BMP5 \|EXTL1 \|LEMD1 | −4.03034151 | 0.13595 | 1.12873 | 0.01135 | 0.56173 | 0.92622 |
| MRGPRX4 \|KRTAP5-4 \|ALLC \|C20orf78 \|MSGN1 \|PENK \|IGFL3 \|LEFTY2 \|TMPRSS11E \|EDNRA | −4.04032939 | 0.13855 | 1.31241 | −0.0149 | 0.15838 | 0.92512 |
| CRYGA \|LCE2D \|KAZALD1 \|C8orf74 \|OR10Q1 \|FAM9B \|LCE3E \|DUSP27 \|MOS \|PGLYRP2 | −4.05444457 | 0.15439 | 1.29062 | −0.0275 | −0.6278 | 0.92451 |
| USH1G \|LACRT \|NAT16 \|KRT85 \|LCE2C \|FIBCD1 \|FAM78B \|C1QTNF9B \|BPIFA2 \|LONRF2 | −4.06584826 | 0.15767 | 1.21035 | 0.02338 | 2.86537 | 0.91735 |
| COL6A5 \|GFRAL \|MMP27 \|CADPS2 \|EIF5AL1 \|CNTNAP4 \|ZIC3 \|SST \|THEGL \|GPRIN1 | −4.07625002 | 0.18149 | 1.11477 | −0.0016 | 0.45887 | 0.9433 |
| SLC22A14 \|TMEM56 \|OIT3 \|PRSS55 \|OR10A3 \|ATP12A \|PPIC \|LRRC15 \|SOWAHD \|HOXC5 | −4.08967615 | 0.17293 | 1.14766 | −0.0059 | 0.13827 | 0.92675 |
| IGFL2 \|TMEFF2 \|IGLON5 \|RNF150 \|C16orf78 \|RESP18 \|SNAI2 \|WFDC8 \|CHGB \|OTC | −4.10177165 | 0.17276 | 1.15149 | 0.00766 | −0.8443 | 0.9346 |
| FZD4 \|DLX5 \|PTPRQ \|C10orf90 \|LPAR4 \|C6orf222 \|CACNG4 \|CCL11 \|IFIT1B \|FAM181A | −4.11474905 | 0.13833 | 1.24122 | −0.0129 | 0.30335 | 0.93522 |
| DUOX2 \|FAM3B \|BBOX1 \|LRRD1 \|THRSP \|TMCO5A \|LRRN4CL \|PLA2G5 \|RS1 \|FBXO27 | −4.13495152 | 0.13269 | 1.20902 | 0.00949 | 2.50402 | 0.92442 |
| FLNC \|PNMA2 \|NDST4 \|TPPP \|SFTPA2 \|SH2D7 \|SHCBP1L \|HRH1 \|NT5C1A \|CCL20 | −4.15495438 | 0.15892 | 1.16578 | 0.05679 | 0.12579 | 0.9215 |
| CYP7A1 \|GRM1 \|ALPPL2 \|DPRX \|SOX8 \|OR2F2 \|UGT3A1 \|UGT1A5 \|DLEU2L \|CACNA1B | −4.16899533 | 0.13116 | 1.23271 | 0.02672 | 0.16765 | 0.93197 |
| HMX1 \|BMP10 \|TCTE1 \|FAM189A2 \|SYT6 \|PLA2G2F \|SOX14 \|CCT8L2 \|C11orf40 \|HES7 | −4.18037988 | 0.15804 | 1.15644 | 0.03499 | 2.02077 | 0.92058 |
| CALHM1 \|MMP7 \|POU4F3 \|SYT9 \|DNASE2B \|SSTR2 \|TMEM225 \|HHIPL1 \|EPB41L4B \|DAND5 | −4.19679383 | 0.14557 | 1.1528 | −0.0367 | 2.24759 | 0.92533 |
| MGAT4C \|MTRNR2L4 \|PXT1 \|LHX6 \|DUSP26 \|FER1L6 \|EPYC \|PRR25 \|GNG4 \|RFPL3 | −4.22029067 | 0.1661 | 1.17272 | −0.0209 | 1.63822 | 0.93233 |
| SUN5 \|LIPG \|EPHA3 \|DEFB132 \|MS4A10 \|MRGPRX3 \|SLAMF9 \|OLFML2A \|COLEC10 \|ADRA2B | −4.22831301 | 0.16851 | 1.20387 | −0.0345 | 0.35796 | 0.93013 |
| KLK5 \|CAPN9 \|C1QTNF9 \|SLC39A12 \|XKR3 \|FAM84A \|KCNB1 \|KRTAP3-2 \|TLL2 \|CLEC3A | −4.24753815 | 0.13583 | 1.20149 | 0.01828 | 0.06476 | 0.93383 |
| TM4SF5 \|KLK3 \|NEUROG1 \|GAD2 \|SAPCD2 \|C5orf38 \|SPRR2F \|FAM133A \|MAPK8IP2 \|TMEM235 | −4.25908239 | 0.17233 | 1.04518 | 0.04208 | −0.5997 | 0.90457 |
| CDH5 \|CLLU1OS \|CCL18 \|PRSS45 \|TMEM196 \|TBX22 \|SSX2B \|IL36G \|SNTG1 \|WFDC2 | −4.27844373 | 0.13621 | 1.19729 | 0.04615 | 0.22311 | 0.91931 |
| RD3L \|QRFPR \|GDPD2 \|KIAA1024L \|DACT3 \|PPIAL4G \|THSD7B \|MEPE \|C10orf62 \|TOX3 | −4.29416038 | 0.15814 | 1.1909 | 0.0171 | −0.9618 | 0.93643 |
| CLDN18 \|CCKAR \|STMN4 \|KCNK16 \|CTXN1 \|FRMPD2 \|SLC22A10 \|PTPRN \|EPHA6 \|RBPJL | −4.30651749 | 0.16072 | 1.32791 | −0.0066 | 2.98461 | 0.92877 |
| GPR25 \|IFNA6 \|SCGN \|TMEM139 \|HAND2 \|TGFBR3L \|SLC7A10 \|ADAM29 \|PADI1 \|CALN1 | −4.32539001 | 0.16707 | 1.10034 | 0.0258 | −0.462 | 0.92913 |
| EMID1 \|TMPRSS12 \|OR7D4 \|TM4SF1 \|PNCK \|OR10H5 \|HAO1 \|HTR1A \|ARHGAP40 \|GR1A1 | −4.34533535 | 0.1479 | 1.26686 | 0.02877 | −0.2308 | 0.93139 |
| TGM7 \|SERPINA4 \|DPT \|MAS1L \|RNF224 \|GSX2 \|BCAM \|TNFAIP8L3 \|NDN \|FBXO43 | −4.36448123 | 0.17599 | 1.30501 | −0.0118 | 0.8813 | 0.91724 |
| ADAMTS19 \|GPR32 \|UNC93A \|CLDN5 \|GPR142 \|CNNM1 \|NR5A2 \|CLLU1 \|KLRF2 \|KCNS2 | −4.38239444 | 0.14896 | 1.11916 | 0.06462 | 1.07477 | 0.93562 |
| PMCH \|NPBWR2 \|LZTS1 \|PLA2G2A \|ZNF843 \|SIGLEC8 \|ZNF679 \|KREMEN2 \|KCNG2 \|ASIC4 | −4.40277672 | 0.19036 | 1.12721 | 0.06051 | −0.6443 | 0.9148 |
| SLC22A12 \|CCDC160 \|TACR3 \|SLCO1C1 \|ULBP1 \|FGF14 \|FBP2 \|SCGB2A2 \|SOX18 \|OSR1 | −4.41697516 | 0.14857 | 1.18845 | 0.05386 | 1.39009 | 0.9257 |
| CACNG3 \|PTCHD3 \|SOSTDC1 \|MCHR2 \|PDLIM3 \|C10orf55 \|ASIC2 \|ATP6V0D2 \|ARHGAP36 \|MS4A13 | −4.42902318 | 0.12295 | 1.29151 | 0.00851 | −0.9788 | 0.93568 |
| KCNB2 \|CCDC63 \|ERAS \|MAS1 \|BTG4 \|ANXA13 \|LAPP \|CABP2 \|ANKRD29 \|KCNJ16 | −4.44170159 | 0.1596 | 1.11617 | 0.01275 | 0.99524 | 0.93129 |
| ESM1 \|CNPY1 \|PIWIL3 \|LINC00346 \|ATCAY \|SRCIN1 \|AMTN \|PROK1 \|DLGAP2 \|OR13J1 | −4.45347694 | 0.12332 | 1.20559 | −0.0029 | 3.97664 | 0.93203 |
| KCNK5 \|ACTL10 \|ASB15 \|ART4 \|EFNB2 \|SLC22A6 \|LMO1 \|OR56A1 \|GK2 \|ZSWIM2 | −4.47304146 | 0.20375 | 1.13542 | −0.005 | 1.30249 | 0.93645 |
| MIP \|BPIFB6 \|DIRAS1 \|FRZB \|SMCP \|FEZF1 \|FSHB \|LRRC52 \|NACA2 \|FAM83C | −4.49014954 | 0.13315 | 1.18904 | −0.0113 | 1.38916 | 0.9246 |
| FHL5 \|AIPL1 \|NOS1 \|SH3GL2 \|TUSC1 \|PATE4 \|PLA2G2E \|RBM46 \|CNTD2 \|THBS2 | −4.50732869 | 0.13583 | 1.32877 | −0.0167 | 0.40861 | 0.92925 |
| OR2AT4 \|OTOGL \|TMEM136 \|CCDC8 \|GPR176 \|PCDHA8 \|C9orf50 \|SLITRK5 \|POU4F1 \|ACSM4 | −4.53087951 | 0.19365 | 1.19636 | 0.02333 | 2.07019 | 0.93761 |
| EDN2 \|GKN2 \|ANO3 \|TCP11 \|DOK5 \|NOX1 \|LRRC14B \|CHST9 \|LINGO4 \|C2orf73 | −4.54575298 | 0.14055 | 1.09703 | 0.04337 | 2.02601 | 0.92252 |
| ZNF716 \|ZNF560 \|ASZ1 \|DSG3 \|DIO2 \|ADAMTS3 \|ACTRT1 \|FAM163B \|RIMKLA \|APOL5 | −4.55964866 | 0.14246 | 1.06401 | 0.0795 | −0.0552 | 0.93864 |
| RNF183 \|KRT79 \|FTHL17 \|NOTUM \|PRAP1 \|UCN2 \|GLT6D1 \|TRIM49 \|KLLN \|TSPEAR | −4.57354815 | 0.15918 | 1.26749 | 0.03619 | 0.43677 | 0.90697 |
| WSCD2 \|CYP4A22 \|RMI2 \|FOXD3 \|OTOP2 \|ADAMTS16 \|BPIFB3 \|SPP1 \|HS3ST5 \|CYLC1 | −4.58684502 | 0.17966 | 1.17016 | 0.05464 | 0.20678 | 0.92574 |
| OR4N2 \|IL22RA2 \|B3GNT9 \|TMEM59L \|VSTM2L \|NOXO1 \|RASD2 \|RAB3IL1 \|MOGAT3 \|KCNK12 | −4.60773174 | 0.18084 | 1.23275 | 0.00243 | −0.2098 | 0.90636 |
| FAM218A \|SCARA3 \|CNTER \|RORB \|ZNF280A \|SERPINA12 \|HOXD12 \|MRAP2 \|SATL1 \|CTF1 | −4.62538676 | 0.18178 | 1.14904 | −0.0141 | −0.5742 | 0.9214 |
| CFHR5 \|SSX2 \|OR51I2 \|ANKRD33 \|KERA \|SHISA2 \|FCAMR \|ASB11 \|SYT8 \|ISLR | −4.63606057 | 0.11682 | 1.30753 | 0.00032 | 1.32898 | 0.92352 |
| GRM3 \|SRMS \|CCL13 \|CDH15 \|TMEM82 \|OSR2 \|ADAMTS20 \|GPR101 \|OR2B6 \|FGF8 | −4.65190144 | 0.16954 | 1.10112 | −0.0213 | 0.90543 | 0.91438 |
| TAS1R2 \|CCDC87 \|NES \|DDN \|CABS1 \|PCDHA4 \|TM4SF20 \|OR56A3 \|SLC2A7 \|SLC16A9 | −4.66414385 | 0.16949 | 1.20832 | 0.06064 | −0.464L | 0.92647 |
| MYOCD \|PCDHA5 \|PIR \|GPR158 \|SLC13A1 \|LBX1 \|TEX11 \|TACSTD2 \|LHFPL1 \|RSPO1 | −4.67444471 | 0.17091 | 1.19947 | −0.0139 | 0.84803 | 0.93717 |
| EDA2R \|FLRT2 \|ANKRD65 \|PAX7 \|CSHL1 \|DAPL1 \|KRT71 \|SIX4 \|GUCA1A \|GPRC6A | −4.6973807 | 0.14908 | 1.2918 | −0.0063 | −0.3733 | 0.92327 |
| MAPK4 \|CSPG4 \|FNDC1 \|GJD4 \|HABP2 \|OR2J2 \|F7 \|KCNJ6 \|TTC34 \|SLC35F1 | −4.71048425 | 0.13958 | 1.15124 | 0.06347 | −0.1131 | 0.90856 |
| SLC27A6 \|FADS6 \|GAL3ST3 \|C18orf63 \|LY6G6C \|CBLN4 \|KRT33B \|TPSD1 \|OR51B2 \|OR51B6 | −4.72861645 | 0.12605 | 1.0995 | 0.02231 | −0.9304 | 0.92155 |
| CNIH3 \|ECEL1 \|IL31 \|OR1M1 \|PRDM14 \|EFNA2 \|UNC5C \|EGFLAM \|KRTAP11-1 \|NLRP8 | −4.74961285 | 0.12977 | 1.07742 | 0.03448 | 2.37905 | 0.91662 |
| AADAC \|GPR119 \|MAGEA4 \|SULT1C3 \|NDNF \|NHLH1 \|RASL10B \|TMEM132D \|GABRG1 \|CWH43 | −4.77007722 | 0.14801 | 1.02134 | −0.0262 | −0.1125 | 0.92627 |
| IL20RA \|MATN4 \|OR51S1 \|SIM1 \|LRRC30 \|IRGC \|MYOM3 \|PLA2R1 \|MRAP \|IL20 | −4.78073969 | 0.14714 | 1.29133 | −0.0137 | 0.62941 | 0.92426 |
| C2orf66 \|DSCAM \|TRIM60 \|NEUROD4 \|FAM83B \|BNC1 \|KRTAP5-5 \|PCDHA13 \|RNF180 \|C11orf16 | −4.79528619 | 0.13146 | 1.23778 | −0.0124 | 0.3640L | 0.93358 |
| KRT28 \|OR14I1 \|OR52J3 \|SOX9 \|CMA1 \|CAMK1G \|SYT7 \|OR51F1 \|OR6C76 \|CCNA1 | −4.80596616 | 0.14943 | 1.14144 | −0.0381 | 0.92924 | 0.9375 |
| OR51I1 \|C1orf115 \|PTPRT \|ZFR2 \|CTAG2 \|ASCL2 \|CDRT15L2 \|RTL1 \|C17orf64 \|OR10H4 | −4.82405195 | 0.17436 | 1.27745 | 0.00051 | 6.94036 | 0.92006 |
| CCDC158 \|LHFPL4 \|NTN1 \|HNF4G \|GRM4 \|CPXM2 \|PCDHB1 \|CCDC105 \|SLC6A11 \|KRTAP9-8 | −4.8385281 | 0.13202 | 1.21265 | 0.00946 | 0.38982 | 0.92913 |
| ETV3L \|USP29 \|OBP2B \|CYP2A13 \|FAM71B \|TMEM74 \|KRTAP4-3 \|KLC3 \|PPP1R3C \|DNMT3L | −4.84864003 | 0.14349 | 1.16008 | 0.0166 | 0.62817 | 0.91692 |
| SERPINB4 \|CYP4F11 \|GALNT9 \|OR11L1 \|TINAG \|TRIM42 \|TMEM133 \|OR2H1 \|TM6SF2 \|PCDHB3 | −4.8618376 | 0.12411 | 1.26953 | −0.064L | −0.6416 | 0.93315 |
| PLA2G4E \|GUCA1C \|PRSS46 \|RASL12 \|SPEM1 \|PRAMEF12 \|PDE4C \|COL20A1 \|ACR \|TSPAN11 | −4.88308893 | 0.12942 | 1.27978 | −0.0096 | 1.26896 | 0.90836 |
| OR4C3 \|CREB3L1 \|OR11H6 \|OR51F2 \|PCDHA11 \|OR10A4 \|FGG \|OR6B3 \|OR6K3 \|OR6S1 | −4.8987934 | 0.14155 | 1.28156 | −0.033 | −0.5148 | 0.93603 |
| KCNA1 \|CHST1 \|TMEM92 \|C11orf53 \|SRL \|ZNF837 \|RNF222 \|IRX5 \|LRRIQ4 \|SYT12 | −4.91111188 | 0.18587 | 1.10823 | 0.03692 | −0.7876 | 0.9205 |
| FAM26E \|PCDHA10 \|CAMKV \|SLITRK6 \|UPK1A \|ADAMTS9 \|TAAR1 \|DLX2 \|SLC22A11 \|TDRD5 | −4.93280596 | 0.18732 | 1.13863 | 0.06749 | 1.19116 | 0.93528 |
| OR51G2 \|PURG \|CCL15 \|SPAM1 \|PRTN3 \|OR6N1 \|OR2W1 \|OR6V1 \|HOXD9 \|VGF | −4.94600477 | 0.1957 | 1.23673 | 0.05714 | −0.7706 | 0.92874 |
| PRNT \|NOBOX \|SLC6A14 \|FGF18 \|ALPI \|IGSF21 \|UGT1A4 \|OR2K2 \|NEURL1B \|CXorf36 | −4.96037452 | 0.134 | 1.19393 | 0.06223 | −0.0109 | 0.92739 |
| ITLN2 \|APOA5 \|BPIFB2 \|VWC2L \|PIWIL1 \|TMEM200C \|CTNNA2 \|NRTN \|WSCD1 \|KLHL30 | −4.98340817 | 0.16632 | 1.33942 | −0.0319 | −0.041 | 0.91285 |
| BARX2 \|GLIS1 \|C10orf120 \|FAM198A \|INHBC \|MYL1 \|ORSP2 \|OR2D3 \|HTR1D \|RAD51AP2 | −5.0050232 | 0.13943 | L.14686 | 0.00588 | 2.45206 | 0.9416 |
| KCNA10 \|NAA11 \|KLK10 \|CDH19 \|PRIMA1 \|HOXD1 \|BARHL2 \|ZSCAN4 \|MC3R \|VAX1 | −5.02432452 | 0.14908 | 1.20084 | −0.0353 | 0.89227 | 0.92598 |
| OR9Q2 \|ARHGEF16 \|CAPZA3 \|OR6T1 \|OR10J1 \|DND1 \|FCRL4 \|MEO1I \|FOXG1 \|KIF12 | −5.03848238 | 0.15618 | 1.36101 | −0.022 | 1.66266 | 0.93227 |
| FGF17 \|TMEM61 \|KIF26A \|FSTL5 \|TNNI1 \|MMP3 \|NHLRC4 \|NR0B1 \|NPTX1 \|SPATS1 | −5.05442835 | 0.159 | 1.14633 | −0.0066 | −0.7479 | 0.90999 |
| GULP1 \|ADRB3 \|C2orf54 \|OR2D2 \|NPFFR1 \|SPATA4 \|HOXB5 \|OCSTAMP \|XKR4 \|HPN | −5.07042729 | 0.19266 | 1.17388 | −0.0072 | 0.10556 | 0.91446 |
| TWIST2 \|KCNV1 \|KRTAP4-11 \|SV2B \|KCNJ9 \|SLC7A2 \|NLRP11 \|PCDHAC1 \|FLRT3 \|TLX2 | −5.08961861 | 0.15613 | 1.15429 | 0.00394 | 0.8744 | 0.933 |
| GH2 \|DACT2 \|FGF5 \|KRT84 \|BARHL1 \|ANKRD34C \|ANKRD63 \|GJA3 \|MXRA5 \|NCR2 | −5.1102684 | 0.16388 | 1.22071 | 0.05782 | 0.99026 | 0.91159 |
| LRRC73 \|STOML3 \|DRD1 \|HOGA1 \|GLP1R \|HOXA11 \|STXBP6 \|ANKRD9 \|LRTM1 \|KRT77 | −5.13519518 | 0.18588 | 1.22624 | −0.0471 | 1.31684 | 0.92186 |
| C1orf158 \|VSTM4 \|FAM196A \|APOH \|TRIM67 \|RND2 \|TMPRSS11B \|MSX2 \|PADI3 \|GPR62 | −5.15794465 | 0.18036 | 1.08182 | 0.02863 | −0.5005 | 0.92591 |
| NKX6-1 \|CYP1A1 \|OR5A1 \|LMX1B \|UGT1A1 \|OR5AU1 \|ESPNL \|TCP10 \|CAV3 \|PRMT8 | −5.17834839 | 0.15583 | 1.20036 | 0.02861 | 1.02446 | 0.91682 |
| FBXO17 \|ZBTB8B \|CTXN3 \|RBP3 \|PABPC1L2A \|HOXD13 \|SERPINB12 \|THSD7A \|GNAT1 \|LEP | −5.19999685 | 0.15714 | 1.14788 | 0.01032 | −0.4376 | 0.9256 |
| HS3ST2 \|CRX \|C1QL1 \|CCDC74B \|DCAF4L2 \|HOXD8 \|IGSF1 \|PSD2 \|GJA8 \|KRTAP4-1 | −5.22416049 | 0.18122 | 1.20121 | 0.02119 | −0.6896 | 0.92168 |
| CHRNB3 \|GCNT3 \|NCAN \|SOST \|ADCY8 \|FAM46B \|TEX13B \|DLL4 \|IL17REL \|KLHL34 | −5.23799833 | 0.15838 | 1.28631 | 0.0236 | −0.4602 | 0.90861 |
| TFPI2 \|LHX3 \|PPP3R2 \|ACE2 \|PPP2R2C \|EN2 \|CDH12 \|KCNK3 \|PABPC5 \|CASKIN1 | −5.25120023 | 0.15589 | 1.02962 | 0.03203 | −0.1132 | 0.9146 |
| DMRTA1 \|SYT16 \|APCDD1L \|SERPINB5 \|KIAA1210 \|C8A \|CCDC33 \|PRLHR \|SLC13A2 \|PGR | −5.26619531 | 0.14298 | 1.27387 | 0.00507 | 0.71561 | 0.93781 |
| POM121L12 \|POU4F2 \|KRT33A \|DPPA2 \|GRID1 \|IL1RAPL2 \|WNT5A \|SPATA12 \|METTL7B \|OPCML | −5.28986165 | 0.16381 | 1.21759 | −0.0311 | −0.7633 | 0.91542 |
| DACH2 \|DEXI \|TEX101 \|CTAG1B \|OR4N4 \|IGF2BP1 \|CCDC166 \|RTP3 \|RHO \|KRT80 | −5.31342506 | 0.17128 | 1.25266 | 0.00682 | −0.565 | 0.92292 |
| ARNT2 \|SULT4A1 \|PDYN \|TRPC4 \|PATE3 \|SLC25A41 \|LMX1A \|CNTN3 \|SEC14L4 \|ITGB6 | −5.33731262 | 0.12039 | 1.07289 | −0.0053 | −0.2776 | 0.9311 |
| PDX1 \|TEX13A \|CLSTN2 \|BCAR1 \|FAM110C \|ZNRF4 \|CDH16 \|LRCH2 \|CLVS2 \|CAMK2N2 | −5.3568467 | 0.18049 | 1.03077 | 0.02892 | −0.6146 | 0.90666 |
| HTRA3 \|CPA2 \|NPBWR1 \|TTLL6 \|FBLL1 \|GAL3ST2 \|OR6A2 \|C8orf48 \|NEUROG3 \|GOT1L1 | −5.37340042 | 0.15711 | 1.25882 | 0.00764 | 1.17451 | 0.92697 |
| DBX1 \|CYP2C18 \|OR2C1 \|MRGPRE \|CYP11B2 \|GALR1 \|SORCS1 \|PLCXD3 \|OR6B2 \|RNF128 | −5.41084756 | 0.09342 | 1.25438 | 0.0001 | 1.45791 | 0.92133 |
| CREG2 \|OXTR \|VGLL3 \|ERVMER34-1 \|GRM6 \|TEKT4 \|OTOL1 \|CHST6 \|GPR12 \|ZNF423 | −5.43417041 | 0.16955 | 1.05128 | 0.03878 | 1.39244 | 0.92096 |
| FGF13 \|FAT3 \|ALX1 \|MTRNR2L6 \|REN \|KLBL1 \|TMEM207 \|GDF7 \|ATP2B3 \|IL22RA1 | −5.4499211 | 0.14928 | 1.10744 | 0.05734 | −0.3985 | 0.93986 |
| PCDHA1 \|LCN15 \|CHRDL1 \|ALX4 \|AGBL1 \|MYF6 \|EPHA7 \|PAPPA2 \|NKPD1 \|LRRC4C | −5.46831333 | 0.15983 | 1.21078 | −0.0014 | 0.20887 | 0.92706 |
| MADCAM1 \|SPACA1 \|TUBAL3 \|COL4A6 \|FRRS1L \|BRS3 \|SPHKAP \|MMP16 \|KCNJ12 \|AMOTL2 | −5.50429854 | 0.16291 | 1.16665 | −0.0131 | −0.8232 | 0.92708 |
| PTCHD4 \|ELMOD1 \|PRSS33 \|RNASE13 \|MYOG \|PRR19 \|MAGEB2 \|MTRNR2L1 \|SERPINB3 \|MYF5 | −5.52422624 | 0.15223 | 1.25852 | −0.0266 | 0.87036 | 0.925 |
| UGT2A2 \|PAPOLB \|MESP2 \|MGAT5B \|SIX1 \|FAM155A \|FSCB \|NTSR2 \|IRX6 \|NLRP13 | −5.5498613 | 0.17137 | 1.26431 | −0.0529 | 0.89175 | 0.92971 |
| AADACL4 \|QRFP \|PRELP \|COX6B2 \|PATE2 \|VIP \|C1QTNF4 \|CEACAM16 \|PRKACG \|PCDH10 | −5.56865296 | 0.15462 | 1.18781 | −0.0158 | 0.03734 | 0.92651 |
| INHBB \|UGT2A1 \|C2orf78 \|CD276 \|KANK4 \|HCAR1 \|BMX2 \|TMEM158 \|CCL16 \|BHLHE22 | −5.60390795 | 0.18182 | 1.19903 | 0.01349 | −0.0426 | 0.92667 |
| PCDHGA2 \|OR13C2 \|KRT36 \|CHDH \|FGF16 \|KRTAP24-1 \|C17orf74 \|OPALIN \|OPN4 \|SLC22A24 | −5.62443024 | 0.13877 | 1.26245 | −0.0045 | −0.382 | 0.93379 |
| NPHS2 \|OPRD1 \|KRT39 \|TMEM125 \|CCDC172 \|FOXI2 \|RHOV \|MAGEA1 \|FAM216B \|SMTNL2 | −5.66400968 | 0.14765 | 1.14264 | 0.00276 | 4.81653 | 0.92641 |
| BPIFB1 \|RNF182 \|KRT20 \|TEX19 \|SFT2D3 \|GPR88 \|C14orf37 \|KRT32 \|KRT34 \|POU3F2 | −5.69612666 | 0.17572 | 1.26047 | −0.0109 | −0.4092 | 0.927 |
| WNT2 \|TRIM63 \|CYP4A11 \|IGDCC3 \|CHST3 \|NDUFC2-KCTD14 \|GLRA4 \|LRRC55 \|TRIML1 \|MIXL1 | −5.72803321 | 0.16449 | 1.10136 | 0.03936 | 4.58585 | 0.91326 |
| RBMXL3 \|ZNF648 \|SLC26A10 \|PAQR9 \|KIAA1644 \|PGLYRP4 \|HCRTR2 \|FABP1 \|KRT4 \|VANGL2 | −5.75828826 | 0.15504 | 1.09455 | 0.06309 | 0.83996 | 0.91756 |
| FOXF1 \|TLX1NB \|HEPACAM2 \|TCF21 \|BGN \|NGB \|LHFPL5 \|TUSC5 \|HGFAC \|SRRM3 | −5.79220743 | 0.14206 | 1.01219 | 0.00123 | 1.4801 | 0.90606 |
| HIN1 \|SLC30A10 \|SLC17A4 \|CPLX1 \|DMRTB1 \|AFP \|KCNG3 \|KIRP1 \|FBN3 \|SLC9A2 | −5.81500355 | 0.16517 | 1.09212 | 0.04124 | −0.0374 | 0.92875 |
| IFNW1 \|C17orf102 \|FIBIN \|NANOS1 \|GPR6 \|HAS2 \|GPR50 \|ENPP6 \|NOX4 \|VAT1L | −5.8348942 | 0.16089 | 1.17967 | −0.0127 | 0.56468 | 0.92052 |
| GDF2 \|PSG4 \|LBP \|MUM1L1 \|GAB4 \|ZIM2 \|ELFN1 \|PNPLA5 \|KLHL38 \|SEMA5B | −5.84911595 | 0.12639 | 1.27525 | 0.00826 | 5.33534 | 0.92251 |
| XKR7 \|GRXCR2 \|TMEM145 \|BIRC8 \|LYPD4 \|HSPA12A \|STC1 \|GABRD \|MRGPRX2 \|NLRP5 | −5.88394881 | 0.15646 | 1.02182 | −0.0166 | −0.2495 | 0.92718 |
| RIPK4 \|RXRG \|NPB \|UNCX \|CLDN23 \|GPR31 \|SCN2B \|NMUR2 \|SOX7 \|WNT9A | −5.90486538 | 0.19606 | 1.01491 | 0.06145 | 5.05664 | 0.92436 |
| C3orf70 \|WIPF3 \|TM4SF4 \|LRIT1 \|OR11A1 \|IL1A \|ADRA2C \|C1orf94 \|ASCL4 \|ACTC1 | −5.93998287 | 0.17007 | 1.10842 | 0.06837 | 0.28875 | 0.92809 |
| IVL \|ANKS4B \|ATXN3L \|KCNA4 \|DMRT3 \|OR51A7 \|FOXF2 \|FGF10 \|CDH6 \|GDF10 | −5.97450289 | 0.1341 | 1.23288 | −0.048 | 1.05318 | 0.93194 |
| OR2AG2 \|NPR1 \|MAGEE2 \|AGTR1 \|C20orf144 \|ACTL7B \|SPRY4 \|CES5A \|CLEC14A \|KCNA7 | −6.01077168 | 0.19365 | 1.21562 | 0.00294 | −0.1316 | 0.92875 |
| GPIHBP1 \|AGT \|STEAP2 \|ISLR2 \|GJ82 \|BPIFA3 \|GATA5 \|FZD9 \|NKX2-1 \|B3GALT5 | −6.04424762 | 0.15973 | 1.16818 | 0.02317 | 1.09335 | 0.9187 |
| TIGD4 \|CRTAC1 \|PNLIPRP3 \|LRRC31 \|CLDN4 \|MMD2 \|EGR4 \|PAMR1 \|ZIC5 \|PLA2G3 | −6.07403349 | 0.17721 | 1.17984 | −0.0075 | −0.1549 | 0.92484 |
| HOXC13 \|KIAA1755 \|PRDM13 \|DCDC2 \|MYADML2 \|NPAS4 \|IRX2 \|EN1 \|HEPH \|PRKAA2 | −6.10839148 | 0.19661 | 1.01933 | 0.05791 | 0.89247 | 0.91816 |
| FOXC2 \|ALDH3B2 \|OR2C3 \|PCDH20 \|TYR \|MAGEA10 \|SLC17A7 \|PTPN5 \|PBOV1 \|CDK5R2 | −6.12918311 | 0.1354 | 1.10303 | 0.05353 | −0.2964 | 0.92734 |
| ST8SIA3 \|FAM47A \|PLA2G4D \|FIGN \|HMGCLL1 \|GABBR2 \|SOX1 \|PAQR5 \|SP9 \|PCDHA9 | −6.14957139 | 0.1703 | 1.19924 | −0.0079 | 0.03134 | 0.9313 |
| PCDHA12 \|CCDC3 \|TNN \|KCNG4 \|OR51E2 \|ZFP42 \|GAS1 \|CNGA2 \|C10orf71 \|TECTB | −6.19595776 | 0.12588 | 1.26125 | 0.01403 | 0.72196 | 0.93314 |
| DCLK3 \|SLC30A2 \|FAXC \|LRRC3 \|OR52E4 \|ARC \|GREL3 \|CDCP2 \|INSM1 \|INSM2 | −6.25918586 | 0.18481 | 1.17977 | −0.0022 | 0.65146 | 0.91902 |
| CPN2 \|C1orf64 \|MEP1A \|OLFM4 \|FAM163A \|HTR5A \|RASSF9 \|ACHE \|FOXB1 \|SLC1A6 | −6.30305347 | 0.16847 | 1.32062 | 0.03512 | −0.0153 | 0.92665 |
| VSX2 \|LUM \|ASB5 \|SOWAHA \|EFNB3 \|BOD1L2 \|LGB \|OR10A6 \|SAMD5 \|ZG16 | −6.35588186 | 0.1531 | 1.22904 | 0.04363 | −0.1706 | 0.92618 |
| HTR1F \|SDC1 \|KCNMB2 \|FAM71E2 \|ZNF536 \|SOWAHB \|RNF152 \|RAB3B \|LRAT \|FAM47C | −6.40166795 | 0.17039 | 1.23447 | −0.0636 | 0.51577 | 0.92745 |
| FZD10 \|PDZRN4 \|SALL3 \|ZNF488 \|OGDHL \|PABPC3 \|ZDHHC22 \|HAPLN4 \|VRTN \|NPY2R | −6.46010418 | 0.17058 | 1.10249 | 0.05482 | 1.33342 | 0.91694 |
| MBL2 \|RPTN \|BMP2 \|OR51E1 \|SYPL2 \|HEYL \|AADACL3 \|GUCY2F \|CCDC85A \|SLC10A2 | −6.53239978 | 0.18108 | 1.2636 | 0.03434 | −0.3101 | 0.92971 |
| ELOVL2 \|OR11G2 \|FZD7 \|FAM181B \|OR52A5 \|ONECUT2 \|FRMPD4 \|TMEM47 \|MAGEL2 \|PSAPL1 | −6.60923587 | 0.18742 | 0.99139 | 0.05243 | 0.7508 | 0.91952 |
| TRIM72 \|RGS7BP \|KIAA0087 \|CILP2 \|CHRD \|CHIA \|RD3 \|ADCY1 \|ELFN2 \|FAM135B | −6.66793619 | 0.16234 | 1.00549 | 0.03405 | 1.38596 | 0.91316 |
| PCDHA3 \|ADIPOQ \|SYTL5 \|PRDM6 \|JPH2 \|TMEM178B \|FGF12 \|TMEM132C \|OR6B1 \|SLC5A7 | −6.77723678 | 0.14872 | 1.10848 | 0.04429 | −0.4245 | 0.92448 |
| SLCO2B1 \|LIN28B \|BMP3 \|NAT8L \|RGS8 \|HRK \|ZBED3 \|KBTBD11 \|MEX3A \|C15orf59 | −6.92592865 | 0.19388 | 1.05799 | 0.00971 | −0.0322 | 0.91254 |
| PCDB7 \|NBLH2 \|HCN4 \|PCDB19 \|TRIM71 \|ONECUT3 \|OR8D1 \|KRTAP3-3 \|LYZL2 \|PRAMEF10 | −7.31132618 | 0.14874 | 1.37631 | −0.0507 | 0.96092 | 0.93182 |
| DUX4L2 \|DUX4L3 \|DUX4L5 \|DUX4L6 \|CDY2A \|A4GNT \|AADACL2 \|ABCG4 \|ABED16B \|ACTBL2 | −7.99999889 | 0.18601 | 1.32783 | 0.0267 | −0.9523 | 0.93068 |
| ACTL8 \|ACTL9 \|ACTRT2 \|ACTRT3 \|ADAM2 \|ADAMTS15 \|ADCYAP1 \|ADRA1B \|ADRA1D \|AGTR2 | −8 | 0.13509 | 1.30276 | −0.0461 | 0.62785 | 0.91866 |
| AIF1L \|ALDH3A1 \|ALX3 \|AMELY \|AMYIB \|ANHX \|ANKRD2 \|ANKRD7 \|APLN \|APLNR | −8 | 0.1768 | 1.04546 | 0.09979 | 0.48759 | 0.91191 |
| APOA4 \|APOC3 \|AQP12A \|AQP6 \|ARL14 \|ARSF \|ARSI \|ART5 \|ASB10 \|ASB17 | −8 | 0.12088 | 1.2286 | −0.0465 | −0.7932 | 0.90753 |
| ASB18 \|ASCL1 \|ATOH1 \|ATOH7 \|ATP5EP2 \|ATP6V1G3 \|AVP \|AVPR1B \|B3GALT1 \|B3GNT6 | −8 | 0.15454 | 1.2607 | 0.00787 | −0.601 | 0.93315 |
| BANF2 \|BARX1 \|BDKRB1 \|BDKRB2 \|BHLHA9 \|BHLHE23 \|BLID \|BMP15 \|BPIFA1 \|BSND | −8 | 0.12864 | 1.13502 | 0.02673 | 3.48153 | 0.92246 |
| BSX \|BTBD17 \|C10orf111 \|C10orf82 \|C11orf52 \|C11orf86 \|C11orf87 \|C11orf91 \|C14orf177 \|C14orf180 | −8 | 0.1712 | 1.06228 | −0.0079 | 2.76292 | 0.91134 |
| C15orf32 \|C16orf92 \|C17orf112 \|C17orf82 \|C19orf81 \|C1orf185 \|C1orf210 \|C1orf226 \|C1orf229 \|C1QL2 | −8 | 0.14151 | 1.30466 | −0.0266 | 0.2381 | 0.91662 |
| C1QL4 \|C1QTNF2 \|C1QTNF8 \|C20orf141 \|C20orf202 \|C20orf85 \|C2CD4A \|C2CD4B \|C2CD4C \|C2orf27B | −8 | 0.17825 | 1.2093 | 0.023 | 0.42075 | 0.9132 |
| C2orf72 \|C2orf83 \|C3orf36 \|C3orf79 \|C3orf80 \|C5orf46 \|C5orf47 \|C5orf49 \|C5orf52 \|C6orf141 | −8 | 0.18576 | 1.2183 | −0.0008 | 1.50752 | 0.93563 |
| C6orf15 \|C6orf223 \|C7orf33 \|C7orf65 \|C7oxf66 \|C8orf4 \|C8orf86 \|C9orf129 \|C9orf152 \|C9orf163 | −8 | 0.15678 | 1.216 | 0.00968 | 0.53363 | 0.92741 |
| C9orf170 \|C9orf62 \|CACNG5 \|CACNG6 \|CACNG7 \|CALML3 \|CALML5 \|CAPN6 \|CASP12 \|CBLN2 | −8 | 0.15234 | 1.18468 | 0.03614 | 0.61828 | 0.90681 |
| CCDC140 \|CCDC169-SOHLH2 \|CCK \|CCKBR \|CCL17 \|CCL19 \|CCL21 \|CCL22 \|CCL24 \|CCL26 | −8 | 0.16362 | 1.13222 | 0.05167 | 0.19665 | 0.90951 |
| CCL7 \|CCNO \|CD300LG \|CD5L \|CDC42EP5 \|CDH22 \|CDKL4 \|CDR2L \|CDSN \|CDX1 | −8 | 0.16709 | 1.14255 | −0.0103 | −0.059 | 0.9259 |
| CDX2 \|CDX4 \|CEMP1 \|CER1 \|CETN1 \|CFC1B \|CGB8 \|CH25H \|CHMP4C \|CHRM1 | −8 | 0.15316 | 1.17299 | 0.09239 | 0.41635 | 0.91774 |
| CERNA9 \|CIDEA \|CITED4 \|CLDN14 \|CLDN17 \|CLDN22 \|CLDN6 \|CLDN8 \|CLPSL2 \|CNTNAP5 | −8 | 0.19206 | 1.26557 | 0.01321 | 0.33738 | 0.93667 |
| CORT \|COX6A2 \|COX7B2 \|COX8C \|CPE \|CPXCR1 \|CPZ \|CRABP1 \|CRCT1 \|CRH | −8 | 0.15176 | 1.15684 | 0.02626 | −0.0973 | 0.92665 |
| CRHR1 \|CRISP3 \|CRNN \|CRYAA \|CRYBA2 \|CRYGD \|CST1 \|CST2 \|CST4 \|CST5 | −8 | 0.10862 | 1.2109 | 0.00756 | 1.29577 | 0.92129 |
| CST8 \|CST9 \|CSTYL \|CSTL1 \|CT45A5 \|CT62 \|CTRB1 \|CTXN2 \|CX3CL1 \|CXCL13 | −8 | 0.13429 | 1.33257 | 0.01371 | 1.17923 | 0.92346 |
| CXCL6 \|CYGB \|CYP11A1 \|CYP4B1 \|CYS1 \|DBX2 \|DCAF12L1 \|DCAF12L2 \|DCAF8L1 \|DDIT4L | −8 | 0.18015 | 1.13965 | 0.05014 | 0.70089 | 0.91405 |
| DDX53 \|DEFA1B \|DEFA4 \|DEFB118 \|DEFB121 \|DEFB123 \|DEFB124 \|DEFB126 \|DEFB135 \|DEFB136 | −8 | 0.11593 | 1.24258 | 0.01966 | 1.46442 | 0.92946 |
| DEFB4B \|DIO3 \|DIRAS2 \|DIRAS3 \|DIRC1 \|DKFZP434A062 \|DKK1 \|DLX3 \|DMBX1 \|DMRTA2 | −8 | 0.13964 | L.1857 | −0.0163 | −0.1099 | 0.92953 |
| DNAJB8 \|DNAJC22 \|DNAJC5G \|DPYSL5 \|DSCR4 \|DUSP13 \|DUSP21 \|DUSP9 \|DUX4L4 \|EBF2 | −8 | 0.20211 | 1.29773 | 0.00396 | −0.115 | 0.91536 |
| ECSCR \|EFNA3 \|EFS \|EMILIN3 \|EPHA8 \|EPO \|ESX1 \|EVX1 \|EVX2 \|FABP4 | −8 | 0.17232 | 0.9869 | 0.07496 | 2.04144 | 0.90941 |
| FABP9 \|FAM110D \|FAM131C \|FAM162B \|FAM167B \|FAM170B \|FAM180A \|FAM19A3 \|FAM19A5 \|FAM215A | −8 | 0.15588 | 1.15652 | 0.06798 | −0.3184 | 0.91288 |
| FAM24A \|FAM25G \|FAM26D \|FAM43B \|FAM46D \|FAM47B \|FAM69C \|FAM71C \|FAM71E1 \|FAM89A | −8 | 0.16432 | 1.20244 | 0.00381 | 0.32 | 0.92424 |
| FATE1 \|FBXL7 \|FCN3 \|FERD3L \|FETUB \|FEV \|FGF1 \|FGF19 \|FGF21 \|FGF3 | −8 | 0.15212 | 1.19634 | 0.04449 | 3.84869 | 0.91482 |
| FGF4 \|FGF7 \|FGFBP1 \|FJX1 \|FMR1NB \|FOXA3 \|FOXB2 \|FOXL2 \|FOXQ1 \|FOXR2 | −8 | 0.18711 | 1.35187 | −0.0168 | −0.7994 | 0.92347 |
| FOXS1 \|FRMD1 \|FTMT \|FUT5 \|FUT9 \|FXYD6-FXYD2 \|GABRP \|GABRQ \|GAL \|GALR3 | −8 | 0.17202 | 1.11186 | 0.05155 | 3.2757 | 0.90259 |
| GAST \|GATA4 \|GBX2 \|GCG \|GDF3 \|GFRA3 \|GERA4 \|GJB1 \|GJB4 \|GJD3 | −8 | 0.15436 | 1.23672 | −0.0103 | 0.35866 | 0.91779 |
| GMNC \|GNAT3 \|GNG12 \|GNGT1 \|GP2 \|GPR139 \|GPR148 \|GPR149 \|GPR17 \|GPR26 | −8 | 0.14155 | 1.28668 | −0.0101 | 0.39414 | 0.92928 |
| GPR37 \|GPR4 \|GPR78 \|GPRIN2 \|GPX8 \|GREM2 \|GRP \|GSC2 \|GSX1 \|GTSF1L | −8 | 0.1464 | 1.16157 | 0.01197 | 3.48791 | 0.91406 |
| GUCA2B \|GYG2 \|B2AFB1 \|H2BFM \|B2BFWT \|HAND1 \|HAPLN1 \|HAPLN2 \|HAS1 \|HBZ | −8 | 0.13765 | 1.25902 | 0.04376 | 0.26325 | 0.91946 |
| HCRT \|HDGFL1 \|HELT \|HEPN1 \|HES3 \|HES5 \|HEY1 \|HIGD2B \|HIST1H2AA \|HIST1H2BA | −8 | 0.18513 | 1.28453 | −0.008 | −0.7171 | 0.92501 |
| HIST1H4K \|HMGB4 \|HMX3 \|HOXA13 \|HOXB1 \|HOXB8 \|HOXB9 \|HOXC11 \|HOXC12 \|HOXC6 | −8 | 0.18189 | 1.00644 | 0.01608 | 1.96052 | 0.92366 |
| HOXC8 \|BOXC9 \|HOXD11 \|HOXD3 \|HOXD4 \|HS3ST6 \|HS6ST2 \|HS6ST3 \|HSPB2 \|HTN3 | −8 | 0.15841 | 1.17864 | −0.005 | 3.37952 | 0.91048 |
| HTR1B \|HTR1E \|HTR2C \|HTR3D \|HYAL4 \|ID4 \|IFNA14 \|IFNA5 \|IGFBP1 \|IGFBPL1 | −8 | 0.15086 | 1.26235 | −0.0081 | 4.20359 | 0.92874 |
| IL17D \|I17F \|IL1F10 \|IL21 \|IL25 \|IL3 \|IL36B \|IL36RN \|INA \|INHA | −8 | 0.13805 | 1.26193 | 0.06509 | 6.14685 | 0.92422 |
| INS-IGF2 \|INSL5 \|IP6K3 \|IQCF5 \|IQCF6 \|IQCJ \|IRX1 \|IRX3 \|IRX4 \|KAAG1 | −8 | 0.11853 | 1.2662 | −0.019 | 2.01895 | 0.91477 |
| KBTBD13 \|KCNF1 \|KCNH6 \|KCNJ4 \|KCNK15 \|KCNK4 \|KCTD14 \|KCTD16 \|KCTD8 \|KHDC3L | −8 | 0.17731 | 1.15232 | −0.066 | 2.81088 | 0.90978 |
| KIF25 \|KIF2B \|KISS1 \|KLF17 \|KLHDC7A \|KLK13 \|KLK6 \|KLK7 \|KLK8 \|KLK9 | −8 | 0.10144 | 1.26307 | 0.0331 | 3.36775 | 0.90264 |
| KLRG2 \|KRT25 \|KRT27 \|KRT35 \|KRT37 \|KRT38 \|KRT40 \|KRT82 \|KRTAP1-1 \|KRTAP1-3 | −8 | 0.08708 | 1.31149 | −0.0054 | 3.5305 | 0.92815 |
| KRTAP-5 \|KRTAP10-1 \|KRTAP10-10 \|KRTAP10-5 \|KRTAP10-6 \|KRTAP12-2 \|KRTAP12-3 \|KRTAP12-4 \|KRTAP1 | −8 | 0.13199 | 1.24653 | 0.00519 | 2.16146 | 0.92025 |
| KRTAP13-4 \|KRTAP17-1 \|KRTAP19-1 \|KRTAP19-8 \|KRTAP2-2 \|KRTAP20-2 \|KRTAP20-4 \|KRTAP22-2 \|KRTAP26 | −8 | 0.06247 | 1.36015 | −0.013 | −0.96 | 0.94227 |
| KRTAP4-2 \|KRTAP4-7 \|KRTAP4-8 \|KRTAP4-9 \|KRTAP5-11 \|KRTAP5-3 \|KRTAP6-3 \|KRTAP8-1 \|KRTAP9-2 \|KRT [symbol] | −8 | 0.09515 | 1.36973 | −0.010S | 2.44074 | 0.92638 |
| KRTAP9-9 \|LAD1 \|LCE1A \|LCE1B \|LCE1C \|LCE1F \|LCE2B \|LCE3A \|LCE3C \|LCE4A | −8 | 0.08618 | 1.2002 | 0.00039 | 2.40201 | 0.92443 |
| LCE6A \|LCN1 \|LCN6 \|LCN9 \|LGALS7B \|LHX2 \|LHX5 \|LHX8 \|LINGO1 \|LOR | −8 | 0.11921 | 1.20845 | −0.0245 | 0.0947 | 0.90384 |
| LRIT2 \|LRRC10 \|LRRC10B \|LRRC24 \|LRRC26 \|LRRC38 \|LRRC3B \|LRRC8E \|LRRTM1 \|LRRTM3 | −8 | 0.16562 | 1.08019 | 0.01605 | 1.74804 | 0.91999 |
| LRTM2 \|LUZP4 \|LY6D \|LY6G6D \|LY6H \|LYPD6 \|LYZL4 \|MAFA \|MAGEA12 \|MAGEA6 | −8 | 0.15558 | 1.04922 | 0.05844 | −0.6744 | 0.90127 |
| MAGEA9 \|MAGEB1 \|MAGEB16 \|MAGEB18 \|MAGEB3 \|MAGEB4 \|MAGEB6 \|MAGEC2 \|MAP1LC3C \|MB | −8 | 0.13654 | 1.30035 | 0.00205 | −0.6679 | 0.92607 |
| MBD3L1 \|MBD3L3 \|MC2R \|MC4R \|MDFI \|METTL11B \|MKX \|MNX1 \|MOGAT2 \|MRGPRG | −8 | 0.16379 | 1.16078 | 0.00714 | −0.0345 | 0.9268 |
| MS4A15 \|MSMB \|MSMP \|MT1B \|MT1M \|MT4 \|MTNR1A \|MTRNR2L10 \|MTRNR2L5 \|MYL10 | −8 | 0.12674 | 1.23015 | 0.03076 | 3.09936 | 0.92652 |
| MYLK2 \|MYOD1 \|NANOS2 \|NANOS3 \|NAP1L6 \|NCCRP1 \|NEU2 \|NEUROG2 \|NKAIN1 \|NKAIN3 | −8 | 0.14839 | 1.1197 | −0.0138 | 0.12073 | 0.90124 |
| NKAIN4 \|NKD2 \|NKX1-2 \|NKX2-2 \|NKX2-3 \|NKX2-4 \|NKX2-5 \|NKX2-6 \|NKX2-8 \|NKX3-2 | −8 | 0.16744 | 0.93545 | 0.12757 | 3.36828 | 0.9052 |
| NKX6-3 \|NMBR \|NMS \|NNMT \|NOTO \|NOV \|NOVA2 \|NPPC \|NPTX2 \|NPVF | −8 | 0.1651 | 1.17067 | 0.0561 | 3.3813 | 0.92031 |
| NPY \|NPY1R \|NPY5R \|NR2F1 \|NRARP \|NRSN1 \|NTF4 \|NTS \|NXNL1 \|NXPH2 | −8 | 0.17329 | 1.22036 | 0.05565 | −0.4304 | 0.92576 |
| NYX \|OBP2A \|CC90 \|OCM2 \|ODF1 \|ODF3L2 \|ODF4 \|OPRK1 \|OR10H2 \|OR12D2 | −8 | 0.10902 | 1.05928 | 0.06267 | 1.84431 | 0.91912 |
| OR13C3 \|OR14A16 \|OR1B1 \|OR1C1 \|OR1L3 \|OR2AK2 \|OR282 \|OR2S2 \|OR278 \|OR2V1 | −8 | 0.10489 | 1.18172 | −0.0312 | 2.5701 | 0.93827 |
| OR4M2 \|OR51T1 \|OR52B4 \|OR52E2 \|OR52E8 \|OR52M1 \|OR52N5 \|OR5AK2 \|OR5B2 \|OR5D18 | −8 | 0.1327 | 1.11845 | 0.05635 | −0.7423 | 0.94013 |
| OR5H6 \|OR5M11 \|OR5M8 \|OR5V1 \|OR6F1 \|OR6M1 \|OR6X1 \|OR7E24 \|OR8A1 \|OR8B12 | −8 | 0.10829 | 1.24184 | −0.018 | 1.03529 | 0.93723 |
| OR8D4 \|OTOP1 \|OTOP3 \|OTOS \|OTP \|OTUD6A \|OVOL1 \|OVOL2 \|PABPC1L2B \|PABPC4L | −8 | 0.17373 | 1.17485 | 0.03721 | 0.23477 | 0.91716 |
| PAGE1 \|PAGE2 \|PAGE5 \|PALM2 \|PCDH8 \|PCDHA2 \|PCK1 \|PCP4 \|PCSK1N \|PFN3 | −8 | 0.15582 | 1.17967 | 0.0041 | 0.50575 | 0.93739 |
| PHLDA3 \|PHOX2B \|PHYHIP \|PHYHIPL \|PIRT \|PITX3 \|PKDCC \|PLA2G12B \|PLAC1 \|PLK5 | −8 | 0.16848 | 1.10896 | 0.07195 | 1.12326 | 0.92045 |
| PLSCR5 \|PMP2 \|POU3F1 \|POU3F3 \|POU3F4 \|PPP1R1C \|PPP1R3G \|PRAMEF2 \|PRDM12 \|PRLH | −8 | 0.17117 | 1.15579 | −0.0326 | 0.37131 | 0.933 |
| PRND \|PRODH2 \|PROKR1 \|PROP1 \|PRPH \|PRPS1L1 \|PRR15 \|PRR16 \|PRR18 \|PRR23A | −8 | 0.16418 | 1.27515 | −0.021 | 0.49698 | 0.9168 |
| PRR23B \|PRR23C \|PRRX2 \|PRSS38 \|PRSS56 \|PSORS1C2 \|PTHLH \|PYDC1 \|PYY \|R3HDML | −8 | 0.16873 | 1.25666 | 0.01134 | −0.8258 | 0.90813 |
| RBM24 \|RBMY1F \|REG1A \|REG3A \|REG3G \|REM1 \|REP15 \|RFPL1 \|RFPL4B \|RGS9BP | −8 | 0.15401 | 1.18995 | −0.0101 | 0.7356 | 0.93006 |
| RHBDL3 \|RBBG \|RHCG \|RIMS4 \|RIPPLY1 \|RIPPLY2 \|RNF113B \|RNF133 \|RNF186 \|RNF223 | −8 | 0.1673 | 1.08016 | −0.0085 | −0.4442 | 0.91464 |
| RPL10L \|RPL3L \|RPRM \|RSPO2 \|RTN4RL1 \|RTP1 \|RTP2 \|RXFP3 \|S100A14 \|S100A16 | −8 | 0.16913 | 1.23696 | 0.03991 | 0.047 | 0.917 |
| S100A7A \|SAA4 \|SAGE1 \|SCGB1D2 \|SCGB2A1 \|SCRT1 \|SCRT2 \|SEMA3F \|SERINC4 \|SFRP1 | −8 | 0.19844 | 1.2604 | 0.00388 | 1.83114 | 0.91106 |
| SFTA2 \|SHISA6 \|SHISA7 \|SHOX2 \|SHROOM2 \|SIAH3 \|SIX2 \|SIX6 \|SLC15A5 \|SLC17A6 | −8 | 0.18596 | 1.13823 | 0.00373 | 2.24963 | 0.92874 |
| SLC18A3 \|SLC22A8 \|SLC32A1 \|SLC35D3 \|SLC6A18 \|SLC6A19 \|SLC6A7 \|SLC8A2 \|SLITRK1 \|SLITRK2 | −8 | 0.14556 | 1.12357 | 0.02837 | 2.35524 | 0.89569 |
| SLN \|SNCB \|SOD3 \|SOHLH1 \|SOX10 \|SOX17 \|SOX21 \|SOX3 \|SP6 \|SPACA4 | −8 | 0.15806 | 1.15383 | 0.0372 | −0.57 | 0.90744 |
| SPANXC \|SPANXN1 \|SPANXN4 \|SPATA19 \|SPATA8 \|SPDEF \|SPERT \|SPHAR \|SPINK6 \|SPINK8 | −8 | 0.11497 | 1.39405 | −0.0517 | −0.5053 | 0.92826 |
| SPPL2C \|SPRR1A \|SPRR1B \|SPRR2A \|SPRR3 \|SPRR4 \|SPSB4 \|SSTR1 \|SSTR4 \|SSX1 | −8 | 0.1108 | 1.14806 | −0.0211 | 0.72241 | 0.93141 |
| ST6GALNAC5 \|STARD6 \|STATH \|SULT1E1 \|SULT6B1 \|SYCE3 \|SYCN \|SYNDIG1 \|SYNGR4 \|SYT13 | −8 | 0.19435 | 1.13586 | 0.02217 | −0.3333 | 0.92772 |
| SYT3 \|TAAR2 \|TAC1 \|TAS2R1 \|TBC1D21 \|TBC1D26 \|TBC1D28 \|TBC1D3 \|TBR1 \|TBX18 | −8 | 0.14863 | 1.06988 | 0.08178 | −0.218 | 0.93671 |
| TBX2 \|TCEAL7 \|TCF15 \|TCF23 \|TCF24 \|TFAP2C \|TFDP3 \|TFF1 \|TFF2 \|TGIF2LX | −8 | 0.1602 | 1.16639 | 0.03074 | −0.1312 | 0.91638 |
| TIMP4 \|TLX1 \|TLX3 \|TM4SF18 \|TMEM100 \|TMEM151A \|TMEM179 \|TMEM215 \|TMEM229A \|TMEM233 | −8 | 0.15141 | 1.25163 | 0.03251 | 0.97467 | 0.9272 |
| TMEM239 \|TMEM247 \|TMEM54 \|TMEM88B \|TMSB15A \|TNFRSF6B \|TNFSF18 \|TNMD \|INP1 \|TP53AIP1 | −8 | 0.15369 | 1.28636 | −0.0151 | 0.62354 | 0.91458 |
| TPBGL \|TPD52L3 \|TPSG1 \|TRABD28 \|TRAM1L1 \|TRH \|TRIM43 \|TRNP1 \|TRPC7 \|TSLP | −8 | 0.17144 | 1.07518 | 0.00398 | 1.23737 | 0.92545 |
| TSPY2 \|TSPY3 \|TSSK1B \|TSSK2 \|TUBA3C \|TUBB3 \|TWIST1 \|TXNRD3NB \|UBD \|UBE2QL1 | −8 | 0.16354 | 1.22459 | −0.0185 | 0.37575 | 0.90765 |
| UBL4B \|UCMA \|UCN3 \|UGT1A3 \|UGT1A6 \|URGCP-MRPS24 \|UTF1 \|UTS2R \|VAX2 \|VCX | −8 | 0.14483 | 1.1942 | −0.0426 | 0.08342 | 0.92603 |
| VCX2 \|VCY \|VCY1B \|VGLL1 \|VGLL2 \|VHLL \|VN1R4 \|VPS37D \|VWA1 \|VWC2 | −8 | 0.18986 | 0.89901 | 0.09575 | −0.7454 | 0.92161 |
| WFDC10A \|WFDC10B \|WFDC13 \|WIF1 \|WISP2 \|WNT3 \|WNT3A \|WNT6 \|WNT7B \|WNT8A | −8 | 0.14478 | 0.99342 | 0.02467 | 0.09496 | 0.91903 |

[symbol]

indicates data missing or illegible when filed

TABLE 5
TSSs in the EPIC-seq selector. Each row corresponds to
one TSS in the EPIC-seq sequencing panel (‘selector’).

| | | TSS | Gene | | |
|---|---|---|---|---|---|
| Hugo symbol | Chromosome | [hg19] | strand | Category | TSS ID |
| BPNT1 | chr1 | 220263191 | −1 | GCB | BPNT1_1 |
| FAM46C | chr1 | 118148603 | 1 | ABC | FAM46C_1 |
| ITPKB | chr1 | 226926876 | −1 | GCB | ITPKB_1 |
| KCNA3 | chr1 | 111217655 | −1 | ABC | KCNA3_1 |
| SLAMF1 | chr1 | 160617081 | −1 | GCB | SLAMF1_1 |
| CD1B | chr1 | 158301321 | −1 | Positive Control | CD1B_1 |
| CD1C | chr1 | 158259562 | 1 | Positive Control | CD1C_1 |
| CD1E | chr1 | 158323485 | 1 | Positive Control | CD1E_1 |
| CHI3L1 | chr1 | 203155922 | −1 | Positive Control | CHI3L1_1 |
| FCGR3B | chr1 | 161601252 | −1 | Positive Control | FCGR3B_1 |
| FCGR3B | chr1 | 161601753 | −1 | Positive Control | FCGR3B_2 |
| LCK | chr1 | 32716839 | 1 | Positive Control | LCK_1 |
| LCK | chr1 | 32739711 | 1 | Positive Control | LCK_2 |
| RGS13 | chr1 | 192605267 | 1 | Positive Control | RGS13_1 |
| BCL2L15 | chr1 | 114430169 | −1 | LUAD | BCL2L15_1 |
| MUC1 | chr1 | 155162706 | −1 | LUAD | MUC1_1 |
| CLCA2 | chr1 | 86889768 | 1 | LUSC | CLCA2_1 |
| IRF6 | chr1 | 209979520 | −1 | LUSC | IRF6_1 |
| PKP1 | chr1 | 201252579 | 1 | LUSC | PKP1_1 |
| S100A2 | chr1 | 153538306 | −1 | LUSC | S100A2_1 |
| S100A7 | chr1 | 153433137 | −1 | LUSC | S100A7_1 |
| SFN | chr1 | 27189632 | 1 | LUSC | SFN_1 |
| APOBEC4 | chr1 | 183622448 | −1 | negativeControl | APOBEC4_1 |
| TNNT2 | chr1 | 201346805 | −1 | negativeControl | TNNT2_1 |
| ASB13 | chr10 | 5708558 | −1 | GCB | ASB13_1 |
| BLNK | chr10 | 98031273 | −1 | ABC | BLNK_1 |
| BLNK | chr10 | 98031333 | −1 | ABC | BLNK_2 |
| ENTPD1 | chr10 | 97471535 | 1 | ABC | ENTPD1_1 |
| ENTPD1 | chr10 | 97515408 | 1 | ABC | ENTPD1_2 |
| ENTPD1 | chr10 | 97515672 | 1 | ABC | ENTPD1_3 |
| SFTPA1 | chr10 | 81370694 | 1 | LUAD | SFTPA1_1 |
| SFTPA2 | chr10 | 81320163 | −1 | LUAD | SFTPA2_1 |
| SFTPD | chr10 | 81708861 | −1 | LUAD | SFTPD_1 |
| CALML3 | chr10 | 5566923 | 1 | LUSC | CALML3_1 |
| CYB5R2 | chr11 | 7694821 | −1 | ABC | CYBSR2_1 |
| LMO2 | chr11 | 33891371 | −1 | GCB | LMO2_1 |
| LMO2 | chr11 | 33891509 | −1 | GCB | LMO2_2 |
| LMO2 | chr11 | 33913836 | −1 | GCB | LMO2_3 |
| CXCR5 | chr11 | 118764100 | 1 | Positive Control | CXCR5_1 |
| MS4A1 | chr11 | 60223281 | 1 | Positive Control | MS4A1_1 |
| P2RY2 | chr11 | 72929343 | 1 | Positive Control | P2RY2_1 |
| P2RY2 | chr11 | 72929501 | 1 | Positive Control | P2RY2_2 |
| TYR | chr11 | 88911039 | 1 | Positive Control | TYR_1 |
| FOLR1 | chr11 | 71900601 | 1 | LUAD | FOLR1_1 |
| FOLR1 | chr11 | 71900958 | 1 | LUAD | FOLR1_2 |
| FOLR1 | chr11 | 71903172 | 1 | LUAD | FOLR1_3 |
| MUC5B | chr11 | 1244294 | 1 | LUAD | MUC5B_1 |
| MUC6 | chr11 | 1036706 | −1 | LUAD | MUC6_1 |
| TRIM29 | chr11 | 120008863 | −1 | LUSC | TRIM29_1 |
| CCND2 | chr12 | 4382901 | 1 | ABC | CCND2_1 |
| ETV6 | chr12 | 11802787 | 1 | ABC | ETV6_1 |
| HSP90B1 | chr12 | 104324188 | 1 | ABC | HSP90B1_1 |
| LRMP | chr12 | 25205180 | 1 | GCB | LRMP_1 |
| PMCH | chr12 | 102591614 | −1 | Positive Control | PMCH_1 |
| ST8SIA1 | chr12 | 22487648 | −1 | Positive Control | ST8SIA1_1 |
| SCNN1A | chr12 | 6484390 | −1 | LUAD | SCNN1A_1 |
| SCNN1A | chr12 | 6484905 | −1 | LUAD | SCNN1A_2 |
| SCNN1A | chr12 | 6486523 | −1 | LUAD | SCNN1A_3 |
| KRT5 | chr12 | 52914243 | −1 | LUSC | KRT5_1 |
| KRT6A | chr12 | 52887181 | −1 | LUSC | KRT6A_1 |
| NDUFA4L2 | chr12 | 57634475 | −1 | LUSC | NDUFA4L2_1 |
| FOXO1 | chr13 | 41240734 | −1 | DLBCLpath | FOXO1_1 |
| BATF | chr14 | 75988783 | 1 | ABC | BATF_1 |
| DAAM1 | chr14 | 59655380 | 1 | GCB | DAAM1_1 |
| DAAM1 | chr14 | 59730158 | 1 | GCB | DAAM1_2 |
| FUT8 | chr14 | 65877309 | 1 | ABC | FUT8_1 |
| FUT8 | chr14 | 65879447 | 1 | ABC | FUT8_2 |
| SERPINA9 | chr14 | 94942670 | −1 | GCB | SERPINA9_1 |
| GZMB | chr14 | 25103432 | −1 | Positive Control | GZMB_1 |
| GZMH | chr14 | 25078864 | −1 | Positive Control | GZMH_1 |
| TCL1A | chr14 | 96180533 | −1 | Positive Control | TCL1A_1 |
| NKX2-1 | chr14 | 36988903 | −1 | LUAD | NKX2-1_1 |
| NKX2-1 | chr14 | 36989430 | −1 | LUAD | NKX2-1_2 |
| RGS6 | chr14 | 72398816 | 1 | LUSC | RGS6_1 |
| RGS6 | chr14 | 72399155 | 1 | LUSC | RGS6_2 |
| RGS6 | chr14 | 72399785 | 1 | LUSC | RGS6_3 |
| BMF | chr15 | 40398287 | −1 | ABC | BMF_1 |
| BMF | chr15 | 40398639 | −1 | ABC | BMF_2 |
| BMF | chr15 | 40401075 | −1 | ABC | BMF_3 |
| IL16 | chr15 | 81517639 | 1 | ABC | IL16_1 |
| AQP9 | chr15 | 58430407 | 1 | Positive Control | AQP9_1 |
| GCNT3 | chr15 | 59903981 | 1 | LUAD | GCNT3_1 |
| ITPKA | chr15 | 41786055 | 1 | LUAD | ITPKA_1 |
| IRF8 | chr16 | 85932773 | 1 | DLBCLpath | IRF8_1 |
| TPSAB1 | chr16 | 1290677 | 1 | Positive Control | TPSAB1_1 |
| C16orf89 | chr16 | 5116146 | −1 | LUAD | C16orf89_1 |
| MT1X | chr16 | 56716381 | 1 | LUSC | MT1X_1 |
| IKZF3 | chr17 | 38020441 | −1 | DLBCLpath | IKZF3_1 |
| ALOX15 | chr17 | 4544960 | −1 | Positive Control | ALOX15_1 |
| ITGA3 | chr17 | 48133339 | 1 | LUAD | ITGA3_1 |
| KRT13 | chr17 | 39661865 | −1 | LUSC | KRT13_1 |
| KRT15 | chr17 | 39675270 | −1 | LUSC | KRT15_1 |
| KRT16 | chr17 | 39769079 | −1 | LUSC | KRT16_1 |
| KRT17 | chr17 | 39780882 | −1 | LUSC | KRT17_1 |
| ANKFN1 | chr17 | 54230835 | 1 | negativeControl | ANKFN1_1 |
| MYL4 | chr17 | 45286713 | 1 | negativeControl | MYL4_1 |
| TCF4 | chr18 | 52969852 | −1 | ABC | TCF4_1 |
| TCF4 | chr18 | 52989090 | −1 | ABC | TCF4_2 |
| TCF4 | chr18 | 53071226 | −1 | ABC | TCF4_3 |
| TCF4 | chr18 | 53089723 | −1 | ABC | TCF4_4 |
| TCF4 | chr18 | 53178000 | −1 | ABC | TCF4_5 |
| TCF4 | chr18 | 53255860 | −1 | ABC | TCF4_6 |
| TCF4 | chr18 | 53257045 | −1 | ABC | TCF4_7 |
| DSC3 | chr18 | 28622781 | −1 | LUSC | DSC3_1 |
| DSG3 | chr18 | 29027731 | 1 | LUSC | DSG3_1 |
| SERPINB13 | chr18 | 61254533 | 1 | LUSC | SERPINB13_1 |
| ARID3A | chr19 | 926036 | 1 | ABC | ARID3A_1 |
| SPIB | chr19 | 50922194 | 1 | ABC | SPIB_1 |
| TCF3 | chr19 | 1650286 | −1 | DLBCLpath | TCF3_1 |
| CLC | chr19 | 40228669 | −1 | Positive Control | CLC_1 |
| FFAR2 | chr19 | 35940616 | 1 | Positive Control | FFAR2_1 |
| NKG7 | chr19 | 51875960 | −1 | Positive Control | NKG7_1 |
| CXCL17 | chr19 | 42947136 | −1 | LUAD | CXCL17_1 |
| ICAM1 | chr19 | 10381516 | 1 | LUAD | ICAM1_1 |
| NAPSA | chr19 | 50868931 | −1 | LUAD | NAPSA_1 |
| SLC1A6 | chr19 | 15083730 | −1 | LUSC | SLC1A6_1 |
| CCL20 | chr2 | 228678557 | 1 | Positive Control | CCL20_1 |
| CTLA4 | chr2 | 204732510 | 1 | Positive Control | CTLA4_1 |
| ICOS | chr2 | 204801470 | 1 | Positive Control | ICOS_1 |
| ZAP70 | chr2 | 98330030 | 1 | Positive Control | ZAP70_1 |
| ZAP70 | chr2 | 98350868 | 1 | Positive Control | ZAP70_2 |
| EPAS1 | chr2 | 46524540 | 1 | LUAD | EPAS1_1 |
| SFTPB | chr2 | 85895864 | −1 | LUAD | SFTPB_1 |
| TRPM8 | chr2 | 234826042 | 1 | LUAD | TRPM8_1 |
| GPC1 | chr2 | 241375114 | 1 | LUSC | GPC1_1 |
| ALPP | chr2 | 233243347 | 1 | negativeControl | ALPP_1 |
| WFDC2 | chr20 | 44098393 | 1 | LUAD | WFDC2_1 |
| IGLL3P | chr22 | 25714223 | 1 | Positive Control | IGLL3P_1 |
| ELFN2 | chr22 | 37823505 | −1 | LUAD | ELFN2_1 |
| BCL6 | chr3 | 187452695 | −1 | GCB | BCL6_1 |
| BCL6 | chr3 | 187454285 | −1 | GCB | BCL6_2 |
| BCL6 | chr3 | 187463513 | −1 | GCB | BCL6_3 |
| LPP | chr3 | 187871662 | 1 | GCB | LPP_1 |
| LPP | chr3 | 187930720 | 1 | GCB | LPP_2 |
| LPP | chr3 | 187943192 | 1 | GCB | LPP_3 |
| MME | chr3 | 154797435 | 1 | GCB | MME_1 |
| MME | chr3 | 154797704 | 1 | GCB | MME_2 |
| MME | chr3 | 154797952 | 1 | GCB | MME_3 |
| MME | chr3 | 154798078 | 1 | GCB | MME_4 |
| SH3BP5 | chr3 | 15374136 | −1 | ABC | SH3BP5_1 |
| SLC12A8 | chr3 | 124930243 | −1 | GCB | SLC12A8_1 |
| SLC12A8 | chr3 | 124931609 | −1 | GCB | SLC12A8_2 |
| VGLL4 | chr3 | 11610398 | −1 | GCB | VGLL4_1 |
| VGLL4 | chr3 | 11623836 | −1 | GCB | VGLL4_2 |
| VGLL4 | chr3 | 11762220 | −1 | GCB | VGLL4_3 |
| FOXP1 | chr3 | 71114074 | −1 | DLBCLpath | FOXP1_1 |
| FOXP1 | chr3 | 71180092 | −1 | DLBCLpath | FOXP1_2 |
| FOXP1 | chr3 | 71294316 | −1 | DLBCLpath | FOXP1_3 |
| FOXP1 | chr3 | 71353911 | −1 | DLBCLpath | FOXP1_4 |
| FOXP1 | chr3 | 71592708 | −1 | DLBCLpath | FOXP1_5 |
| FOXP1 | chr3 | 71632904 | −1 | DLBCLpath | FOXP1_6 |
| FOXP1 | chr3 | 71633140 | −1 | DLBCLpath | FOXP1_7 |
| CPA3 | chr3 | 148583042 | 1 | Positive Control | CPA3_1 |
| GPR171 | chr3 | 150920988 | −1 | Positive Control | GPR171_1 |
| HESX1 | chr3 | 57234280 | −1 | Positive Control | HESX1_1 |
| P2RY13 | chr3 | 151047337 | −1 | Positive Control | P2RY13_1 |
| P2RY14 | chr3 | 150966998 | −1 | Positive Control | P2RY14_1 |
| P2RY14 | chr3 | 150996230 | −1 | Positive Control | P2RY14_2 |
| LRRC31 | chr3 | 169587660 | −1 | LUAD | LRRC31_1 |
| CLDN1 | chr3 | 190040235 | −1 | LUSC | CLDN1_1 |
| PFN2 | chr3 | 149688741 | −1 | LUSC | PFN2_1 |
| SOX2 | chr3 | 181328150 | 1 | LUSC | SOX2_1 |
| SOX2 | chr3 | 181429711 | 1 | LUSC | SOX2_2 |
| TP63 | chr3 | 189349215 | 1 | LUSC | TP63_1 |
| TP63 | chr3 | 189507448 | 1 | LUSC | TP63_2 |
| MAPK10 | chr4 | 87028806 | −1 | GCB | MAPK10_1 |
| MAPK10 | chr4 | 87281375 | −1 | GCB | MAPK10_2 |
| MAPK10 | chr4 | 87374283 | −1 | GCB | MAPK10_3 |
| BANK1 | chr4 | 102711763 | 1 | Positive Control | BANK1_1 |
| BANK1 | chr4 | 102734982 | 1 | Positive Control | BANK1_2 |
| CXCL3 | chr4 | 74904490 | −1 | Positive Control | CXCL3_1 |
| CXCL5 | chr4 | 74864416 | −1 | Positive Control | CXCL5_1 |
| HPGDS | chr4 | 95264027 | −1 | Positive Control | HPGDS_1 |
| LEF1 | chr4 | 109087953 | −1 | Positive Control | LEF1_1 |
| LEF1 | chr4 | 109090112 | −1 | Positive Control | LEF1_2 |
| LEF1-AS1 | chr4 | 109088680 | 1 | Positive Control | LEF1-AS1_1 |
| LEF1-AS1 | chr4 | 109093275 | 1 | Positive Control | LEF1-AS1_2 |
| SLC34A2 | chr4 | 25657434 | 1 | LUAD | SLC34A2_1 |
| SLC34A2 | chr4 | 25658085 | 1 | LUAD | SLC34A2_2 |
| FGFBP1 | chr4 | 15940363 | −1 | LUSC | FGFBP1_1 |
| SSBP2 | chr5 | 81047072 | −1 | GCB | SSBP2_1 |
| GZMA | chr5 | 54398473 | 1 | Positive Control | GZMA |
| GZMK | chr5 | 54320106 | 1 | Positive Control | GZMK_1 |
| IL3 | chr5 | 131396346 | 1 | Positive Control | IL3_1 |
| IL9 | chr5 | 135231516 | −1 | Positive Control | IL9_1 |
| TCF7 | chr5 | 133450401 | 1 | Positive Control | TCF7_1 |
| TCF7 | chr5 | 133451297 | 1 | Positive Control | TCF7_2 |
| TCF7 | chr5 | 133451349 | 1 | Positive Control | TCF7_3 |
| SCGB3A2 | chr5 | 147258273 | 1 | LUAD | SCGB3A2_1 |
| ADTRP | chr6 | 11779280 | −1 | ABC | ADTRP_1 |
| CYP39A1 | chr6 | 46620523 | −1 | GCB | CYP39A1_1 |
| MAN1A1 | chr6 | 119670931 | −1 | ABC | MAN1A1_1 |
| PIM1 | chr6 | 37137921 | 1 | ABC | PIM1_1 |
| IRF4 | chr6 | 391738 | 1 | DLBCLpath | IRF4_1 |
| TREM2 | chr6 | 41130922 | −1 | Positive Control | TREM2_1 |
| VNN2 | chr6 | 133084598 | −1 | Positive Control | VNN2_1 |
| ENPP3 | chr6 | 131958441 | 1 | LUAD | ENPP3_1 |
| LGSN | chr6 | 64029882 | −1 | LUAD | LGSN_1 |
| SFTA2 | chr6 | 30899952 | −1 | LUAD | SFTA2_1 |
| FABP7 | chr6 | 123100645 | 1 | LUSC | FABP7_1 |
| PERP | chr6 | 138428660 | −1 | LUSC | PERP_1 |
| CDK14 | chr7 | 90338711 | 1 | GCB | CDK14_1 |
| CREB3L2 | chr7 | 137686847 | −1 | ABC | CREB3L2_1 |
| EGFR | chr7 | 55086724 | 1 | amplificationControl | EGFR_1 |
| MET | chr7 | 116312458 | 1 | amplificationControl | MET_1 |
| MACC1 | chr7 | 20181538 | 1 | LUAD | MACC1_1 |
| MACC1 | chr7 | 20257013 | −1 | LUAD | MACC1_2 |
| FSCN1 | chr7 | 5632435 | 1 | LUSC | FSCN1_1 |
| GPNMB | chr7 | 23286315 | 1 | LUSC | GPNMB_1 |
| HOXA1 | chr7 | 27135625 | −1 | LUSC | HOXA1_1 |
| AGMO | chr7 | 15601640 | −1 | negativeControl | AGMO_1 |
| MYL7 | chr7 | 44180916 | −1 | negativeControl | MYL7_1 |
| DENND3 | chr8 | 142138719 | 1 | GCB | DENND3_1 |
| MYBL1 | chr8 | 67525480 | −1 | GCB | MYBL1_1 |
| PLEKHF2 | chr8 | 96145948 | 1 | GCB | PLEKHF2_1 |
| PTK2 | chr8 | 142011412 | −1 | GCB | PTK2_1 |
| SLA | chr8 | 134072603 | −1 | ABC | SLA_1 |
| SLA | chr8 | 134115310 | −1 | ABC | SLA_2 |
| MYC | chr8 | 128748314 | 1 | amplificationControl | MYC_1 |
| BLK | chr8 | 11351520 | 1 | Positive Control | BLK_1 |
| C8orf4 | chr8 | 40010986 | 1 | LUAD | C8orf4_1 |
| HEY1 | chr8 | 80680098 | −1 | LUSC | HEY1_1 |
| TRPA1 | chr8 | 72987819 | −1 | LUSC | TRPA1_1 |
| RECK | chr9 | 36036909 | 1 | GCB | RECK_1 |
| CCL19 | chr9 | 34691274 | −1 | Positive Control | CCL19_1 |
| CD72 | chr9 | 35618424 | −1 | Positive Control | CD72_1 |
| FCN1 | chr9 | 137809806 | −1 | Positive Control | FCN1_1 |
| AQP3 | chr9 | 33447631 | −1 | LUAD | AQP3_1 |
| GOLM1 | chr9 | 88714510 | −1 | LUAD | GOLM1_1 |
| GOLM1 | chr9 | 88715116 | −1 | LUAD | GOLM1_2 |
| PIM2 | chrX | 48776413 | −1 | ABC | PIM2_1 |
| CLIC2 | chrX | 154563986 | −1 | Positive Control | CLIC2_1 |
| HMGB3 | chrX | 150151762 | 1 | LUAD | HMGB3_1 |
| PLS3 | chrX | 114795176 | 1 | LUAD | PLS3_1 |
| PLS3 | chrX | 114827818 | 1 | LUAD | PLS3_2 |
| SLC6A8 | chrX | 152954965 | 1 | LUSC | SLC6A8_1 |

TABLE 6
EPIC-Seq samples clinical characteristics and scores corresponding to different classifiers. EPIC-Seq was applied to
373 samples, of which 329 passed the QC steps, and were used to show the utility of the inferred gene expression in
different applications: cancer detection, tumor subtype classification, and patient response to treatment prediction

| | | | | Sample | | | | | | | EPIC- |
|---|---|---|---|---|---|---|---|---|---|---|---|
| RunName | patientid | SampleID | Type | labels | AF | Age | Gender | T-MTV | IPI | Stage | lung |
| HiSeq1004 | P-20-0771 | P-20-0771-C1 | cfDNA | LUAD | 4.46 | 65 | F | NA | NA | IV | 1 |
| HiSeq1004 | P-20-0808 | P-20-0808-C1 | cfDNA | LUSC | 14 | 73 | M | NA | NA | IIIB | 1 |
| HiSeq1004 | P-20-0818 | P-20-0818-C1 | cfDNA | LUSC | 0.97 | 80 | M | NA | NA | IV | 1 |
| HiSeq1005 | P-20-0238 | P-20-0238-C1 | cfDNA | LUSC | 2 | 61 | M | 271.54 | NA | IIIA | 1 |
| HiSeq1005 | P-20-0245 | P-20-0245-C1 | cfDNA | LUSC | 7 | 80 | M | 36.77 | NA | IIIA | 1 |
| HiSeq1005 | P-20-0445 | P-20-0445-C1 | cfDNA | LUAD | 0.47 | 80 | M | NA | NA | IV | 1 |
| HiSeq1327 | P-20-0695 | P-20-0695-C1 | cfDNA | LUAD | 3.21 | 76 | M | NA | NA | IV | 1 |
| HiSeq1005 | P-20-0811 | P-20-0811-C1 | cfDNA | LUSC | 2.5 | 60 | M | NA | NA | IIIB | 1 |
| HiSeq1005 | P-20-0822 | P-20-0822-C1 | cfDNA | LUAD | 8.48 | 71 | F | NA | NA | IV | 1 |
| HiSeq1006 | P-20-0247 | P-20-0247-C1 | cfDNA | LUSC | 4 | 76 | M | 148.02 | NA | IIIA | 1 |
| HiSeq1006 | P-20-0781 | P-20-0781-C1 | cfDNA | LUAD | 3.67 | 58 | F | NA | NA | IV | 1 |
| HiSeq1006 | P-20-0790 | P-20-0790-C1 | cfDNA | LUSC | 2.1 | 60 | F | NA | NA | IIIA | 1 |
| HiSeq1006 | P-20-0810 | P-20-0810-C1 | cfDNA | LUSC | 1.4 | 63 | M | 129.3 | NA | IIIA | 1 |
| HiSeq1114 | P-20-0556 | P-20-0556-C1-1114 | cfDNA | LUAD | 9.91 | 61 | F | NA | NA | IV | 1 |
| HiSeq1113 | P-20-0556 | P-20-0556-C2 | cfDNA | LUAD | 0.55 | 61 | F | NA | NA | IV | 0 |
| HiSeq1113 | P-20-0622 | P-20-0622-C4 | cfDNA | LUAD | 18.06 | 41 | F | NA | NA | IV | 0 |
| HiSeq1113 | P-20-0678 | P-20-0678-C1 | cfDNA | LUAD | 10.53 | 65 | F | NA | NA | IV | 1 |
| HiSeq1113 | P-20-0678 | P-20-0678-C2 | cfDNA | LUAD | 1.96 | 65 | F | NA | NA | IV | 0 |
| HiSeq1113 | P-20-0784 | P-20-0784-C4 | cfDNA | LUAD | 0.18 | 68 | M | NA | NA | IV | 1 |
| HiSeq1113 | P-20-0819 | P-20-0819-C1 | cfDNA | LUAD | 0.752 | 76 | F | NA | NA | IV | 1 |
| HiSeq1113 | P-20-0819 | P-20-0819-C2 | cfDNA | LUAD | 5.9 | 76 | F | NA | NA | IV | 0 |
| HiSeq1114 | P-20-0622 | P-20-0622-C1 | cfDNA | LUAD | 7.44 | 41 | F | NA | NA | IV | 1 |
| HiSeq1327 | P-20-0678 | P-20-0678-C4 | cfDNA | LUAD | 0.226 | 65 | F | NA | NA | IV | 0 |
| HiSeq1114 | P-20-0748 | P-20-0748-C1 | cfDNA | LUAD | 1.5 | 75 | M | NA | NA | IV | 1 |
| HiSeq1114 | P-20-0748 | P-20-0748-C2 | cfDNA | LUAD | 0 | 75 | M | NA | NA | IV | 0 |
| HiSeq1114 | P-20-0784 | P-20-0784-C5 | cfDNA | LUAD | 0.249 | 68 | M | NA | NA | IV | 0 |
| HiSeq1144 | P-10-0025 | P-10-0025-C2 | cfDNA | GCB | 1.186224 | 44 | M | NA | 3 | 4 | 0 |
| HiSeq1144 | P-11-0056 | P-11-0056-C1 | cfDNA | ABC | 1.940158 | 60 | M | NA | 3 | 3 | 0 |
| HiSeq1144 | P-11-0230 | P-11-0230-C1 | cfDNA | ABC | 3.522019 | 52 | F | 1464.9 | 4 | 4 | 0 |
| HiSeq1144 | P-11-0246 | P-11-0246-C1 | cfDNA | GCB | 4.252678 | 72 | M | NA | 1 | 2 | 0 |
| HiSeq1144 | P-11-0248 | P-11-0248-C1 | cfDNA | GCB | 1.418782 | 73 | M | NA | 2 | 4 | 0 |
| HiSeq1144 | P-11-0462 | P-11-0462-C1RERUN | cfDNA | GCB | 12.75582 | 67 | F | 355.71 | 4 | 4 | 0 |
| HiSeq1145 | P-11-0074 | P-11-0074-C1V2 | cfDNA | ABC | 1.002884 | 48 | F | NA | 2 | 4 | 0 |
| HiSeq1145 | P-11-0075 | P-11-0075-C1 | cfDNA | ABC | 4.93 | 74 | M | NA | 3 | 4 | 0 |
| HiSeq1145 | P-11-0077 | P-11-0077-C1 | cfDNA | GCB | 5.492853 | 46 | F | NA | 1 | 2 | 0 |
| HiSeq1145 | P-11-0081 | P-11-0081-C1 | cfDNA | GCB | 8.966901 | 49 | F | NA | 1 | 2 | 0 |
| HiSeq1145 | P-11-0236 | P-11-0236-C1 | cfDNA | GCB | 5.934318 | 43 | F | 373.19 | 3 | 4 | 0 |
| HiSeq1145 | P-11-0240 | P-11-0240-C1 | cfDNA | ABC | 1.33 | 74 | F | 42.07 | 1 | 2 | 0 |
| HiSeq1146 | P-10-0029 | P-10-0029-C1-PRE-TX-DG | cfDNA | GCB | 12.03 | 66 | M | NA | 4 | 3 | 0 |
| HiSeq1146 | P-10-0089 | P-10-0089-C2 | cfDNA | GCB | 1.563598 | 73 | F | NA | 2 | 2 | 0 |
| HiSeq1146 | P-10-0120 | P-10-0120-C1 | cfDNA | GCB | 3.74 | 65 | M | NA | 3 | 2 | 0 |
| HiSeq1146 | P-11-0071 | P-11-0071-C1 | cfDNA | GCB | 1.24102 | 64 | F | NA | 3 | 4 | 0 |
| HiSeq1146 | P-11-0223 | P-11-0223-C1 | cfDNA | ABC | 8.993641 | 75 | F | 36.13 | 3 | 4 | 0 |
| HiSeq1146 | P-11-0446 | P-11-0446-C1 | cfDNA | ABC | 20.35623 | 75 | M | 168.96 | 5 | 4 | 0 |
| HiSeq1146 | P-11-0459 | P-11-0459-C1RERUN | cfDNA | GCB | 18.34106 | 49 | M | 696.7 | 3 | 2 | 0 |
| HiSeq1146 | P-11-0461 | P-11-0461-C1RERUN | cfDNA | GCB | 3.429935 | 77 | M | NA | 5 | 3 | 0 |
| HiSeq449 | P-21-0024 | P-21-0024-C1 | cfDNA | LUAD | 30.05 | 57 | F | NA | NA | IV | 1 |
| HiSeq449 | P-21-0025 | P-21-0025-C1 | cfDNA | LUAD | 10.43 | 47 | F | NA | NA | IV | 1 |
| HiSeq449 | P-21-0028 | P-21-0028-C1 | cfDNA | LUAD | 27.58 | 47 | F | NA | NA | IV | 1 |
| HiSeq449 | P-10-0007 | P-10-0007-C14 | cfDNA | GCB | 32.0309 | 42 | F | 868.999 | 1 | 1 | 0 |
| HiSeq449 | P-10-0009 | P-10-0009-1PRE | cfDNA | GCB | 11.45 | 75 | M | 275.439 | 1 | 4 | 0 |
| HiSeq449 | P-10-0032 | P-10-0032-C3 | cfDNA | ABC | 22.29 | 54 | M | 136.47 | 3 | 4 | 0 |
| HiSeq449 | P-10-0033 | P-10-0033-C1PRE | cfDNA | ABC | 7.36101 | 59 | F | NA | 3 | 4 | 0 |
| HiSeq449 | P-20-0296 | P-20-0296-C1 | cfDNA | LUSC | 4.6 | 73 | F | 19.88 | NA | IIA | 1 |
| HiSeq449 | P-20-0530 | P-20-0530-C1 | cfDNA | LUSC | 10.8 | 61 | M | NA | NA | IIIA | 1 |
| HiSeq449 | P-20-0531 | P-20-0531-C1 | cfDNA | LUSC | 6.38 | 80 | F | NA | NA | IIIA | 1 |
| HiSeq449 | P-11-0001 | P-11-0001-C1 | cfDNA | GCB | 27.752 | 50 | F | NA | 3 | 4 | 0 |
| HiSeq449 | P-11-0029 | P-11-0029-C1 | cfDNA | ABC | 18.68 | 83 | M | NA | 1 | 2 | 0 |
| HiSeq508 | P-10-0001 | P-10-0001-C24 | cfDNA | [symbol] ol for noisy | NA | 61 | M | NA | NA | 4 | 0 |
| HiSeq508 | P-10-0009 | P-10-0009-C30 | cfDNA | [symbol] ol for noisy | NA | NA | NA | NA | NA | 4 | 0 |
| HiSeq508 | P-10-0011 | P-10-0011-C13 | cfDNA | [symbol] ol for noisy | NA | 72 | M | 26.819 | NA | 2 | 0 |
| HiSeq508 | P-10-0013 | P-10-0013-C19 | cfDNA | [symbol] ol for noisy | NA | 58 | M | 2046.486 | NA | 2 | 0 |
| HiSeq508 | P-10-0016 | P-10-0016-C23 | cfDNA | [symbol] ol for noisy | NA | 73 | F | 856.177 | NA | 2 | 0 |
| HiSeq508 | P-10-0022 | P-10-0022-C36 | cfDNA | [symbol] ol for noisy | NA | 26 | M | 111.706 | NA | 2 | 0 |
| HiSeq508 | P-10-0026 | P-10-0026-C17 | cfDNA | [symbol] ol for noisy | NA | 80 | M | 15.169 | NA | NA | 0 |
| HiSeq508 | P-10-0032 | P-10-0032-C25 | cfDNA | [symbol] ol for noisy | NA | NA | NA | NA | NA | NA | 0 |
| HiSeq508 | P-10-0042 | P-10-0042-C13 | cfDNA | [symbol] ol for noisy | NA | NA | NA | NA | NA | NA | 0 |
| HiSeq508 | P-10-0043 | P-10-0043-C13 | cfDNA | [symbol] ol for noisy | NA | NA | NA | NA | NA | NA | 0 |
| HiSeq508 | P-10-0044 | P-10-0044-C7 | cfDNA | [symbol] ol for noisy | NA | 68 | M | NA | NA | NA | 0 |
| HiSeq508 | P-10-0045 | P-10-0045-C9 | cfDNA | [symbol] ol for noisy | NA | 35 | F | NA | NA | NA | 0 |
| HiSeq508 | P-10-0049 | P-10-0049-C8 | cfDNA | [symbol] ol for noisy | NA | 44 | F | NA | NA | NA | 0 |
| HiSeq508 | P-10-0050 | P-10-0050-C9 | cfDNA | [symbol] ol for noisy | NA | 46 | M | NA | NA | NA | 0 |
| HiSeq508 | P-10-0051 | P-10-0051-C3 | cfDNA | [symbol] ol for noisy | NA | 64 | F | NA | NA | NA | 0 |
| HiSeq508 | P-10-0052 | P-10-0052-C12 | cfDNA | [symbol] ol for noisy | NA | 68 | M | NA | NA | NA | 0 |
| HiSeq508 | P-10-0056 | P-10-0056-C8 | cfDNA | [symbol] ol for noisy | NA | 70 | F | NA | NA | NA | 0 |
| HiSeq508 | P-10-0059 | P-10-0059-C9 | cfDNA | [symbol] ol for noisy | NA | 32 | F | NA | NA | NA | 0 |
| HiSeq508 | P-10-0060 | P-10-0060-C3 | cfDNA | [symbol] ol for noisy | NA | NA | NA | NA | NA | NA | 0 |
| HiSeq508 | P-10-0066 | P-10-0066-C10 | cfDNA | [symbol] olfor noisy | NA | NA | NA | NA | NA | NA | 0 |
| HiSeq508 | P-10-0069 | P-10-0069-C3 | cfDNA | [symbol] ol for noisy | NA | NA | NA | NA | NA | NA | 0 |
| HiSeq508 | P-10-0071 | P-10-0071-C5 | cfDNA | [symbol] ol for noisy | NA | 77 | F | 5.44 | NA | NA | 0 |
| HiSeq508 | P-10-0072 | P-10-0072-08 | cfDNA | [symbol] ol for noisy | NA | 57 | M | NA | NA | NA | 0 |
| HiSeq508 | P-10-0080 | P-10-0080-C16 | cfDNA | [symbol] ol for noisy | NA | 74 | F | 446.43 | NA | NA | 0 |
| HiSeq618 | P-21-0043 | P-21-0043-C1 | cfDNA | LUAD | 7.36 | 45 | F | NA | NA | IV | 1 |
| HiSeq618 | P-21-0016 | P-21-0016-C1 | cfDNA | LUAD | 10.92 | 48 | F | NA | NA | IV | 1 |
| HiSeq618 | P-21-0033 | P-21-0033-C1 | cfDNA | LUAD | 17.61 | 56 | F | NA | NA | IV | 1 |
| HiSeq618 | P-10-0002 | P-10-0002-1PRE | cfDNA | ABC | 19.94793 | 55 | F | 725.13 | 3 | 4 | 0 |
| HiSeq618 | P-10-0002 | P-10-0002-C24PRETX1STREL | cfDNA | ABC | 13.45 | 55 | F | NA | 3 | 4 | 0 |
| HiSeq618 | P-20-0347 | P-20-0347-C1 | cfDNA | LUSC | 3.44 | 81 | F | 87.17 | NA | IIB | 1 |
| HiSeq618 | P-20-0413 | P-20-0413-C1 | cfDNA | LUSC | 2.98 | 71 | M | NA | NA | IIIA | 1 |
| HiSeq618 | P-20-0450 | P-20-0450-C1 | cfDNA | LUSC | 6.46 | 70 | M | 260.7 | NA | IIIB | 1 |
| HiSeq620 | P-21-0010 | P-21-0010-C1 | cfDNA | LUAD | 27.59 | 55 | F | NA | NA | IV | 0 |
| HiSeq620 | P-21-0005 | P-21-0005-C1 | cfDNA | LUAD | 11.64 | 53 | F | NA | NA | IV | 0 |
| HiSeq620 | P-10-0006 | P-10-0006-8-PRE | cfDNA | ABC | 27.92431 | 54 | M | 11.342 | 4 | 4 | 0 |
| HiSeq620 | P-10-0036 | P-10-0036-C1PRE | cfDNA | GCB | 12.5889 | 61 | M | 3.61 | 2 | 3 | 0 |
| HiSeq620 | P-20-0288 | P-20-0288-C1 | cfDNA | LUSC | 7.55 | 72 | M | NA | NA | IIA | 0 |
| HiSeq620 | P-20-0330 | P-20-0330-C1 | cfDNA | LUSC | 16.62 | 66 | F | NA | NA | IIIB | 0 |
| HiSeq620 | P-20-0742 | P-20-0742-C1 | cfDNA | [symbol] Lthenstage | — | 77 | M | 0.93 | NA | 4 | 0 |
| HiSeq620 | P-11-0212 | P-11-0212-C1 | cfDNA | ABC | 5.837 | 68 | M | NA | 4 | | 0 |
| HiSeq623 | P-10-0005 | P-10-0005-1PRE | cfDNA | ABC | 21.45662 | 58 | M | NA | 4 | 1 | 0 |
| HiSeq623 | P-10-0006 | P-10-0006-1-PRE | cfDNA | ABC | 30.08906 | 54 | M | NA | 4 | 4 | 0 |
| HiSeq623 | P-10-0074 | P-10-0074-C1 | cfDNA | GCB | 13.5722 | 49 | M | 107.75 | 1 | 4 | 0 |
| HiSeq623 | P-10-0082 | P-10-0082-C1 | cfDNA | GCB | 15.38659 | 66 | M | 1115.882 | 5 | 2 | 0 |
| HiSeq623 | P-11-0043 | P-11-0043-C1V2 | cfDNA | GCB | 15.39 | 57 | F | NA | 4 | 1 | 0 |
| HiSeq623 | P-11-0058 | P-11-0058-C1 | cfDNA | ABC | 16.836 | 55 | F | NA | 1 | 3 | 0 |
| HiSeq623 | P-11-0068 | P-11-0068-C1 | cfDNA | ABC | 30.9532 | 63 | M | NA | 4 | 4 | 0 |
| HiSeq623 | P-11-0243 | P-11-0243-C1 | cfDNA | GCB | 29.622 | 68 | M | NA | 3 | 2 | 0 |
| HiSeq623 | P-11-0445 | P-11-0445-C1 | cfDNA | GCB | 12.23 | 27 | M | 1288 | 3 | 2 | 0 |
| HiSeq623 | P-11-0451 | P-11-0451-C1 | cfDNA | GCB | 11.277 | 50 | F | 294.59 | 2 | 4 | 0 |
| HiSeq624 | P-21-0048 | P-21-0048-C1 | cfDNA | LUAD | 17.45 | 51 | F | NA | NA | IV | 1 |
| HiSeq624 | P-10-0031 | P-10-0031-C1 | cfDNA | GCB | 8.245096 | 75 | M | NA | 2 | 4 | 0 |
| HiSeq624 | P-20-0456 | P-20-0456-C1 | cfDNA | LUSC | 4.56 | 63 | F | NA | NA | IIIA | 1 |
| HiSeq724 | P-01-0079 | P-01-0079-C1 | cfDNA | Normal | NA | 41 | F | NA | NA | NA | 1 |
| HiSeq724 | P-01-0081 | P-01-0081-C1 | cfDNA | Normal | NA | 38 | F | NA | NA | NA | 1 |
| HiSeq724 | P-01-0083 | P-01-0083-C1 | cfDNA | Normal | NA | 62 | M | NA | NA | NA | 1 |
| HiSeq724 | P-01-0085 | P-01-0085-C1 | cfDNA | Normal | NA | 66 | F | NA | NA | NA | 1 |
| HiSeq724 | P-01-0086 | P-01-0086-C1 | cfDNA | Normal | NA | 48 | F | NA | NA | NA | 1 |
| HiSeq724 | P-01-0087 | P-01-0087-C1 | cfDNA | Normal | NA | 56 | F | NA | NA | NA | 1 |
| HiSeq724 | P-01-0088 | P-01-0088-C1 | cfDNA | Normal | NA | 44 | M | NA | NA | NA | 1 |
| HiSeq724 | P-01-0089 | P-01-0089-C1 | cfDNA | Normal | NA | 65 | M | NA | NA | NA | 1 |
| HiSeq724 | P-01-0090 | P-01-0090-C1 | cfDNA | Normal | NA | 50 | M | NA | NA | NA | 1 |
| HiSeq724 | P-01-0091 | P-01-0091-C1 | cfDNA | Normal | NA | 31 | F | NA | NA | NA | 1 |
| HiSeq724 | P-01-0092 | P-01-0092-C1 | cfDNA | Normal | NA | 38 | F | NA | NA | NA | 1 |
| HiSeq724 | P-01-0093 | P-01-0093-C1 | cfDNA | Normal | NA | 26 | F | NA | NA | NA | 1 |
| HiSeq804 | P-20-0417 | P-20-0417-C1 | cfDNA | LUSC | 3.61 | 61 | F | NA | NA | IV | 1 |
| HiSeq804 | P-20-0553 | P-20-0553-C1 | cfDNA | LUAD | 0.008251 | 65 | M | NA | NA | IV | 1 |
| HiSeq804 | P-20-0674 | P-20-0674-C1 | cfDNA | LUSC | 1.98 | 56 | M | NA | NA | IV | 0 |
| HiSeq804 | P-20-0676 | P-20-0676-C1 | cfDNA | LUAD | 0.026371 | 73 | M | NA | NA | IV | 1 |
| HiSeq804 | P-20-0684 | P-20-0684-C1 | cfDNA | LUSC | 1.655 | 84 | M | NA | NA | IV | 1 |
| HiSeq804 | P-20-0687 | P-20-0687-C1 | cfDNA | LUSC | 0.014697 | 85 | M | NA | NA | IV | 1 |
| HiSeq841 | P-20-0221 | P-20-0221-C1 | cfDNA | LUSC | 3.87 | 67 | M | 156 | NA | IIIB | 1 |
| HiSeq841 | P-11-0022 | P-11-0022-C1 | cfDNA | GCB | 6.539776 | 38 | M | NA | 2 | 3 | 0 |
| HiSeq841 | P-11-0064 | P-11-0064-C1 | cfDNA | ABC | 5.011 | 78 | M | NA | 2 | 4 | 0 |
| HiSeq841 | P-11-0085 | P-11-0085-C1 | cfDNA | ABC | 0.000574 | 27 | M | NA | 0 | 4 | 0 |
| HiSeq841 | P-11-0241 | P-11-0241-C1 | cfDNA | ABC | 14.08768 | 57 | M | NA | 4 | 2 | 0 |
| HiSeq841 | P-11-0422 | P-11-0422-C1 | cfDNA | GCB | 0.41 | 38 | M | NA | 1 | 4 | 0 |
| HiSeq841 | P-11-0425 | P-11-0425-C1 | cfDNA | GCB | 1.6968 | 54 | F | NA | 2 | 1 | 0 |
| HiSeq841 | P-11-0428 | P-11-0428-C1 | cfDNA | GCB | 0.002519 | 55 | M | NA | 1 | 3 | 0 |
| HiSeq841 | P-11-0429 | P-11-0429-C1 | cfDNA | ABC | 0.386156 | 61 | F | NA | 4 | 4 | 0 |
| HiSeq856 | P-21-0031 | P-21-0031-C1 | cfDNA | LUAD | 10.5696 | 84 | F | NA | NA | IV | 1 |
| HiSeq856 | P-01-0046 | P-01-0046-C1 | cfDNA | Normal | NA | 34 | F | NA | NA | NA | 1 |
| HiSeq856 | P-10-0028 | P-10-0028-C1 | cfDNA | GCB | 3.791739 | 70 | M | NA | 1 | 2 | 0 |
| HiSeq856 | P-20-0241 | P-20-0241-C1 | cfDNA | LUSC | 12.503 | 65 | M | 226.1 | NA | IIIB | 1 |
| HiSeq856 | P-11-0066 | P-11-0066-C1 | cfDNA | GCB | 1.923379 | 68 | M | NA | 3 | 2 | 0 |
| HiSeq856 | P-11-0419 | P-11-0419-C1 | cfDNA | ABC | 0.050599 | 78 | F | 41 | 3 | 2 | 0 |
| HiSeq1327 | P-20-0792 | P-20-0792-C1 | cfDNA | LUAD | 1.6199 | 72 | F | NA | NA | IIB | 1 |
| HiSeq1327 | P-20-0794 | P-20-0794-C1 | cfDNA | LUSC | 1.2363 | 64 | F | NA | NA | IIB | 1 |
| HiSeq1327 | P-20-0809 | P-20-0809-C1 | cfDNA | LUAD | 1.1293 | 68 | M | 22.05 | NA | IIIB | 1 |
| HiSeq1327 | P-20-0840 | P-20-0840-C1 | cfDNA | LUSC | 1.262 | 73 | M | NA | NA | IIIB | 1 |
| HiSeq1326 | P-01-0017 | P-01-0017-C1 | cfDNA | Normal | NA | 66 | M | NA | NA | NA | 1 |
| HiSeq1326 | P-01-0018 | P-01-0018-C2-2 | cfDNA | Normal | NA | 70 | F | NA | NA | NA | 1 |
| HiSeq1326 | P-01-0024 | P-01-0024-C2 | cfDNA | Normal | NA | 45 | M | NA | NA | NA | 1 |
| HiSeq1326 | P-01-0001 | P-01-0001-C1 | cfDNA | Normal | NA | 56 | M | NA | NA | NA | 1 |
| HiSeq1326 | P-01-0026 | P-01-0026-C1-2 | cfDNA | Normal | NA | 56 | M | NA | NA | NA | 1 |
| HiSeq1326 | P-01-0030 | P-01-0030-C3 | cfDNA | Normal | NA | 28 | M | NA | NA | — | 1 |
| HiSeq1326 | P-01-0031 | P-01-0031-C1 | cfDNA | Normal | NA | NA | M | NA | NA | NA | 1 |
| HiSeq1326 | P-01-0034 | P-01-0034-C2 | cfDNA | Normal | NA | 41 | M | NA | NA | — | 1 |
| HiSeq1326 | P-01-0036 | P-01-0036-C1 | cfDNA | Normal | NA | 29 | M | NA | NA | NA | 1 |
| HiSeq1326 | P-01-0043 | P-01-0043-C1 | cfDNA | Normal | NA | 27 | M | NA | NA | — | 1 |
| HiSeq1326 | P-01-0046 | P-01-0046-C2 | cfDNA | Normal | NA | 34 | F | NA | NA | NA | 1 |
| HiSeq1326 | P-01-0047 | P-01-0047-C1 | cfDNA | Normal | NA | 52 | M | NA | NA | — | 1 |
| HiSeq1327 | P-01-0057 | P-01-0057-C1 | cfDNA | Normal | NA | 63 | F | NA | NA | — | 1 |
| HiSeq1326 | P-01-0071 | P-01-0071-C1 | cfDNA | Normal | NA | 26 | M | NA | NA | — | 1 |
| HiSeq1327 | P-01-0072 | P-01-0072-C1 | cfDNA | Normal | NA | 24 | M | NA | NA | — | 1 |
| HiSeq1326 | P-01-0073 | P-01-0073-C1 | cfDNA | Normal | NA | 22 | M | NA | NA | — | 1 |
| HiSeq1327 | P-02-0017 | P-02-0017-C1 | cfDNA | Normal | NA | 65 | F | NA | NA | — | 1 |
| HiSeq1327 | P-02-0018 | P-02-0018-C1 | cfDNA | Normal | NA | 60 | F | NA | NA | — | 1 |
| HiSeq1327 | P-02-0019 | P-02-0019-C1 | cfDNA | Normal | NA | 61 | F | NA | NA | — | 1 |
| HiSeq1327 | P-02-0020 | P-02-0020-C1 | cfDNA | Normal | NA | 68 | M | NA | NA | — | 1 |
| HiSeq1327 | P-02-0021 | P-02-0021-C1 | cfDNA | Normal | NA | 63 | M | NA | NA | — | 1 |
| HiSeq1327 | P-02-0022 | P-02-0022-C1 | cfDNA | Normal | NA | 64 | M | NA | NA | — | 1 |
| HiSeq1363 | P-20-1018 | P-20-1018-C1 | cfDNA | LUAD | 15.89524 | 75 | F | NA | NA | IIIB | 1 |
| HiSeq1363 | P-20-1022 | P-20-1022-C1 | cfDNA | LUSC | 2.082591 | 56 | F | NA | NA | IIIA | 1 |
| HiSeq1363 | P-20-1023 | P-20-1023-C1 | cfDNA | LUSC | 2.833704 | 59 | M | NA | NA | IIIA | 1 |
| HiSeq1363 | P-20-1024 | P-20-1024-C1 | cfDNA | LUSC | 3.55336 | 65 | M | NA | NA | IIIA | 1 |
| HiSeq1363 | P-20-1025 | P-20-1025-C1 | cfDNA | LUAD | 1.361224 | 74 | F | NA | NA | IIIA | 1 |
| HiSeq1363 | P-20-1026 | P-20-1026-C1 | cfDNA | LUAD | 2.44923 | 67 | M | NA | NA | IIIA | 1 |
| HiSeq1363 | P-20-1027 | P-20-1027-C1 | cfDNA | LUSC | 1.160588 | 71 | M | NA | NA | IIIA | 1 |
| HiSeq1363 | P-20-1028 | P-20-1028-C1 | cfDNA | LUAD | 2.199746 | 60 | M | NA | NA | IIIB | 1 |
| HiSeq1363 | P-20-1033 | P-20-1033-C1 | cfDNA | LUAD | 16.75743 | 55 | M | NA | NA | IIIB | 1 |
| HiSeq1364 | P-20-0849 | P-20-0849-C1 | cfDNA | LUAD | 7.892279 | 68 | M | NA | NA | IIIA | 1 |
| HiSeq1364 | P-20-0893 | P-20-0893-C1 | cfDNA | LUSC | 2.139885 | 74 | M | NA | NA | IIIB | 1 |
| HiSeq1364 | P-20-0906 | P-20-0906-C1 | cfDNA | LUAD | 3.298515 | 71 | F | NA | NA | IIIB | 1 |
| HiSeq1364 | P-20-0911 | P-20-0911-C1 | cfDNA | LUSC | 8.397573 | 75 | F | NA | NA | IIB | 1 |
| HiSeq1364 | P-20-0927 | P-20-0927-C1 | cfDNA | LUAD | 19.47437 | 75 | F | NA | NA | IIIA | 1 |
| HiSeq1364 | P-20-0929 | P-20-0929-C1 | cfDNA | LUAD | 3.610177 | 68 | M | NA | NA | IIB | 1 |
| HiSeq1364 | P-20-0933 | P-20-0933-C1 | cfDNA | LUSC | 1.095627 | 78 | M | NA | NA | IIB | 1 |
| HiSeq1364 | P-20-0934 | P-20-0934-C1 | cfDNA | LUSC | 6.556087 | 75 | M | NA | NA | IIIA | 1 |
| HiSeq1363 | P-01-0029 | P-01-0029-C1 | cfDNA | Normal | NA | 45 | M | NA | NA | — | 1 |
| HiSeq1363 | P-01-0010 | P-01-0010-C1 | cfDNA | Normal | NA | 60 | M | NA | NA | NA | 1 |
| HiSeq1363 | P-01-0025 | P-01-0025-C1 | cfDNA | Normal | NA | 57 | M | NA | NA | NA | 1 |
| HiSeq1363 | P-01-0013 | P-01-0013-C3 | cfDNA | exp. model | NA | 29 | M | NA | NA | — | 0 |
| HiSeq1363 | P-01-0084 | P-01-0084-C1 | cfDNA | Normal | NA | 42 | F | NA | NA | — | 1 |
| HiSeq1364 | P-01-0028 | P-01-0028-C1 | cfDNA | Normal | NA | 56 | F | NA | NA | — | 1 |
| HiSeq1364 | P-01-0003 | P-01-0003-C1 | cfDNA | Normal | NA | 65 | F | NA | NA | NA | 1 |
| HiSeq1364 | P-01-0006 | P-01-0006-C1 | cfDNA | Normal | NA | 77 | F | NA | NA | NA | 0 |
| HiSeq1364 | P-01-0009 | P-01-0009-C1 | cfDNA | Normal | NA | 63 | F | NA | NA | NA | 1 |
| HiSeq1364 | P-01-0080 | P-01-0080-C1 | cfDNA | Normal | NA | 30 | F | NA | NA | — | 1 |
| HiSeq1357 | P-11-0442 | P-11-0442-C1 | cfDNA | GCB | 1.289767 | 35 | F | 14.21 | 3 | 3 | 0 |
| HiSeq1357 | P-11-0450 | P-11-0450-C1 | cfDNA | ABC | 6.052239 | 46 | M | 316.7 | 4 | 4 | 0 |
| HiSeq1357 | P-11-0053 | P-11-0053-C1 | cfDNA | ABC | 0.304112 | 68 | M | NA | 2 | 1 | 0 |
| HiSeq1357 | P-11-0455 | P-11-0455-C1 | cfDNA | ABC | 7.009151 | 64 | F | 332.34 | 3 | 3 | 0 |
| HiSeq1357 | P-11-0456 | P-11-0456-C1 | cfDNA | GCB | 4.920662 | 68 | M | 98.75 | 3 | 4 | 0 |
| HiSeq1357 | P-11-0449 | P-11-0449-C1 | cfDNA | ABC | 2.198076 | 44 | F | NA | 2 | 2 | 0 |
| HiSeq1357 | P-11-0460 | P-11-0460-C1 | cfDNA | GCB | 1.651981 | 70 | F | 31.04 | 1 | 2 | 0 |
| HiSeq1357 | P-11-0463 | P-11-0463-C1 | cfDNA | GCB | 0.570421 | 32 | F | 53.06 | 1 | 4 | 0 |
| HiSeq1357 | P-01-0002 | P-01-0002-C1 | cfDNA | Normal | NA | 82 | M | NA | NA | NA | 1 |
| HiSeq1357 | P-01-0016 | P-01-0016-C1 | cfDNA | Normal | NA | 39 | M | NA | NA | NA | 1 |
| HiSeq1357 | P-01-0019 | P-01-0019-C1 | cfDNA | Normal | NA | 28 | M | NA | NA | NA | 1 |
| HiSeq1358 | P-01-0018 | P-01-0018-C1 | cfDNA | Normal | NA | 70 | F | NA | NA | NA | 1 |
| HiSeq1358 | P-11-0070 | P-11-0070-C1 | cfDNA | GCB | 9.145327 | 66 | F | NA | 4 | 2 | 0 |
| HiSeq1358 | P-11-0062 | P-11-0062-C1 | cfDNA | ABC | 7.209943 | 64 | M | NA | 2 | 2 | 0 |
| HiSeq1358 | P-11-0244 | P-11-0244-C1 | cfDNA | ABC | 8.414994 | 80 | F | NA | 3 | 4 | 0 |
| HiSeq1358 | P-11-0037 | P-11-0037-C1 | cfDNA | ABC | 1.047357 | 37 | M | NA | 0 | 2 | 0 |
| HiSeq1358 | P-11-0039 | P-11-0039-C1 | cfDNA | ABC | 3.490454 | 53 | M | NA | 2 | 4 | 0 |
| HiSeq1358 | P-11-0447 | P-11-0447-C1 | cfDNA | ABC | 13.8001 | 61 | M | 1854.85 | 5 | 4 | 0 |
| HiSeq1358 | P-01-0024 | P-01-0024-C1 | cfDNA | Normal | NA | 45 | M | NA | NA | NA | 1 |
| HiSeq1358 | P-11-0044 | P-11-0044-C1 | cfDNA | ABC | 0.625151 | 78 | M | NA | 1 | 2 | 0 |
| HiSeq1359 | P-01-0015 | P-01-0015-C1 | cfDNA | Normal | NA | 32 | M | NA | NA | NA | 1 |
| HiSeq1359 | P-11-0015 | P-11-0015-C1 | cfDNA | GCB | 0.662246 | 44 | M | NA | 0 | 2 | 0 |
| HiSeq1359 | P-11-0072 | P-11-0072-C1 | cfDNA | GCB | 4.509036 | 72 | F | NA | 1 | 2 | 0 |
| HiSeq1359 | P-01-0021 | P-01-0021-C1 | cfDNA | Normal | NA | 30 | M | NA | NA | NA | 1 |
| HiSeq1359 | P-11-0452 | P-11-0452-C1 | cfDNA | GCB | 14.65936 | 73 | F | NA | 4 | 4 | 0 |
| HiSeq1359 | P-11-0080 | P-11-0080-C1 | cfDNA | GCB | 1.079626 | 53 | F | NA | 0 | 2 | 0 |
| HiSeq1360 | P-01-0005 | P-01-0005-C1 | cfDNA | Normal | NA | 65 | F | NA | NA | NA | 1 |
| HiSeq1360 | P-01-0022 | P-01-0022-C1 | cfDNA | Normal | NA | 29 | M | NA | NA | NA | 1 |
| HiSeq1360 | P-01-1005 | P-01-1005-C1 | cfDNA | Normal | NA | 66 | M | NA | NA | NA | 1 |
| HiSeq1360 | P-01-0007 | P-01-0007-C1 | cfDNA | Normal | NA | 59 | F | NA | NA | NA | 1 |
| HiSeq1360 | P-11-0024 | P-11-0024-C1 | cfDNA | ABC | 1.089329 | 77 | M | NA | 3 | 4 | 1 |
| HiSeq1360 | P-11-0040 | P-11-0040-C1 | cfDNA | ABC | 1.27707 | 79 | M | NA | 3 | 4 | 0 |
| HiSeq1360 | P-11-0042 | P-11-0042-C1 | cfDNA | ABC | 19.16926 | 58 | M | NA | 2 | 4 | 0 |
| HiSeq1360 | P-11-0079 | P-11-0079-C1 | cfDNA | GCB | 2.268033 | 84 | F | NA | 1 | 2 | 0 |
| HiSeq1360 | P-11-0073 | P-11-0073-C1 | cfDNA | GCB | 1.340014 | 37 | M | NA | 3 | 4 | 0 |
| HiSeq1360 | P-11-0057 | P-11-0057-C1 | cfDNA | ABC | 0.511012 | 43 | F | NA | 0 | 1 | 0 |
| HiSeq1203 | P-11-0667 | P-11-0667-C1 | cfDNA | GCB | 0.729153 | 72 | M | NA | NA | | 0 |
| HiSeq1203 | P-11-0668 | P-11-0668-C1 | cfDNA | GCB | 3.39125 | 82 | M | NA | 4 | | 0 |
| HiSeq1203 | P-11-0669 | P-11-0669-C1 | cfDNA | ABC | 3.212353 | 77 | F | NA | 3 | | 0 |
| HiSeq1203 | P-10-0111 | P-10-0111-C1 | cfDNA | ABC | 2.375778 | 32 | F | NA | 1 | 2 | 0 |
| HiSeq1440 | P-10-0002 | P-10-0002-C26 | cfDNA | ABC | 21.65637 | 55 | F | NA | NA | | 0 |
| HiSeq1440 | P-10-0002 | P-10-0002-C27 | cfDNA | ABC | 4.260419 | 55 | F | NA | NA | | 0 |
| HiSeq1440 | P-10-0002 | P-10-0002-C28 | cfDNA | ABC | 0.095638 | 55 | F | NA | NA | | 0 |
| HiSeq1440 | P-10-0002 | P-10-0002-C29 | cfDNA | ABC | 6.00E−04 | 55 | F | NA | NA | | 0 |
| HiSeq1440 | P-10-0002 | P-10-0002-C30 | cfDNA | ABC | 0.716419 | 55 | F | NA | NA | | 0 |
| HiSeq1440 | P-10-0007 | P-10-0007-C25 | cfDNA | GCB | 0.630296 | 42 | F | NA | NA | | 0 |
| HiSeq1440 | P-10-0007 | P-10-0007-C26 | cfDNA | GCB | 0.08 | 42 | F | NA | NA | | 0 |
| HiSeq1440 | P-10-0007 | P-10-0007-C27 | cfDNA | GCB | 4.315804 | 42 | F | NA | NA | | 0 |
| HiSeq1440 | P-10-0007 | P-10-0007-C28 | cfDNA | GCB | 32.03087 | 42 | F | NA | NA | | 0 |
| HiSeq1440 | P-10-0007 | P-10-0007-C29 | cfDNA | GCB | 40.90865 | 42 | F | NA | NA | | 0 |
| HiSeq1440 | P-10-0007 | P-10-0007-C30 | cfDNA | GCB | 32.8175 | 42 | F | NA | NA | | 0 |
| HiSeq1440 | P-01-0108 | P-01-0108-C1 | cfDNA | Normal | NA | NA | | NA | NA | | 1 |
| HiSeq1440 | P-01-0133 | P-01-0133-C1 | cfDNA | Normal | NA | 25 | M | NA | NA | | 1 |
| HiSeq1440 | P-01-0135 | P-01-0135-C1 | cfDNA | Normal | NA | 25 | M | NA | NA | | 0 |
| HiSeq1444 | P-02-0058 | P-02-0058-C1 | cfDNA | Normal | NA | 76 | M | NA | NA | | 1 |
| HiSeq1444 | P-02-0011 | P-02-0011-C1 | cfDNA | Normal | NA | 58 | M | NA | NA | | 1 |
| HiSeq1444 | P-02-0061 | P-02-0061-C1 | cfDNA | Normal | NA | 65 | M | NA | NA | | 1 |
| HiSeq1444 | P-02-0016 | P-02-0016-C1 | cfDNA | Normal | NA | 69 | M | NA | NA | | 1 |
| HiSeq1444 | P-02-0042 | P-02-0042-C1 | cfDNA | Normal | NA | 71 | F | NA | NA | | 1 |
| HiSeq1444 | P-02-0009 | P-02-0009-C1 | cfDNA | Normal | NA | 68 | M | NA | NA | | 1 |
| HiSeq1444 | P-20-0855 | P-20-0855-C2 | cfDNA | LUSC | 1.800646 | 84 | M | NA | NA | IV | 0 |
| HiSeq1444 | P-20-1013 | P-20-1013-C1 | cfDNA | LUAD | 0.999232 | 70 | M | NA | NA | IV | 1 |
| HiSeq1444 | P-20-0855 | P-20-0855-C1 | cfDNA | LUSC | 4.150048 | 84 | M | NA | NA | IV | 1 |
| HiSeq1444 | P-20-0853 | P-20-0853-C1 | cfDNA | LUAD | 1.094357 | 83 | M | NA | NA | IV | 1 |
| HiSeq1444 | P-20-0853 | P-20-0853-C2 | cfDNA | LUAD | 0.000897 | 83 | M | NA | NA | IV | 0 |
| HiSeq1444 | P-20-1013 | P-20-1013-C2 | cfDNA | LUAD | 6.156617 | 70 | M | NA | NA | IV | 0 |
| HiSeq1444 | P-20-1014 | P-20-1014-C1 | cfDNA | LUAD | 15.08832 | 58 | M | NA | NA | IV | 1 |
| HiSeq1444 | P-20-1014 | P-20-1014-C2 | cfDNA | LUAD | 6.549028 | 58 | M | NA | NA | IV | 0 |
| HiSeq1444 | P-20-1015 | P-20-1015-C1 | cfDNA | LUAD | 1.286664 | NA | | NA | NA | IV | 1 |
| HiSeq1444 | P-20-1015 | P-20-1015-C2 | cfDNA | LUAD | 0.786582 | NA | | NA | NA | IV | 0 |
| HiSeq1458 | P-11-0655 | P-11-0655-C1 | cfDNA | GCB | 7.962503 | 51 | M | NA | 3 | 4 | 0 |
| HiSeq1458 | P-11-0654 | P-11-0654-C1 | cfDNA | ABC | 6.325708 | 47 | F | NA | 3 | 4 | 0 |
| HiSeq1458 | P-11-0664 | P-11-0664-C1 | cfDNA | ABC | 4.768784 | 73 | M | NA | 4 | 4 | 0 |
| HiSeq1458 | P-11-0680 | P-11-0680-C1 | cfDNA | PMBCL | 1.364339 | 48 | F | NA | 1 | 2 | 0 |
| HiSeq1458 | P-11-0659 | P-11-0659-C1 | cfDNA | ABC | 0.581665 | 32 | F | NA | 2 | 2 | 0 |
| HiSeq1458 | P-11-0676 | P-11-0676-C1 | cfDNA | ABC | 0.440363 | 76 | F | NA | 2 | 3 | 0 |
| HiSeq1458 | P-11-0673 | P-11-0673-C1 | cfDNA | GCB | 4.387583 | 69 | M | NA | 4 | | 0 |
| HiSeq1458 | P-11-0662 | P-11-0662-C1 | cfDNA | ABC | 21.85237 | 35 | F | NA | 1 | 4 | 0 |
| HiSeq1458 | P-11-0609 | P-11-0609-C1 | cfDNA | GCB | 0.56 | 73 | M | NA | 4 | 4 | 0 |
| HiSeq1458 | P-02-0034 | P-02-0034-C1 | cfDNA | exp. model | NA | 76 | F | NA | NA | | 0 |
| HiSeq1458 | P-02-0025 | P-02-0025-C1 | cfDNA | Normal | NA | 63 | F | NA | NA | | 1 |
| HiSeq1458 | P-02-0046 | P-02-0046-C1 | cfDNA | Normal | NA | 69 | F | NA | NA | | 1 |
| HiSeq1458 | P-02-0039 | P-02-0039-C1 | cfDNA | Normal | NA | 74 | M | NA | NA | | 1 |
| HiSeq1458 | P-01-0135 | P-01-0135-C8 | cfDNA | Normal | NA | 26 | M | NA | NA | | 1 |
| HiSeq1459 | P-11-0655 | P-11-0655-C1V2 | cfDNA | GCB | 7.812765 | 51 | M | NA | 3 | 4 | 0 |
| HiSeq1459 | P-11-0660 | P-11-0660-C1 | cfDNA | GCB | 6.260132 | 74 | M | NA | 4 | 4 | 0 |
| HiSeq1459 | P-11-0679 | P-11-0679-C1 | cfDNA | ABC | 4.226071 | 73 | M | NA | 4 | 4 | 0 |
| HiSeq1459 | P-11-0678 | P-11-0678-C1 | cfDNA | ABC | 1.577582 | 76 | F | NA | 3 | 4 | 0 |
| HiSeq1459 | P-11-0652 | P-11-0652-C1 | cfDNA | GCB | 0.800917 | 76 | M | NA | 2 | 4 | 0 |
| HiSeq1459 | P-11-0651 | P-11-0651-C1 | cfDNA | ABC | 0.559354 | 57 | M | NA | 0 | 0 | 0 |
| HiSeq1459 | P-11-0675 | P-11-0675-C1 | cfDNA | GCB | 0.240681 | 37 | M | NA | 1 | | 0 |
| HiSeq1459 | P-11-0656 | P-11-0656-C1 | cfDNA | ABC | 19.73931 | 55 | M | NA | 4 | 2 | 0 |
| HiSeq1459 | P-11-0672 | P-11-0672-C1 | cfDNA | GCB | 17.58043 | 55 | F | NA | 3 | 4 | 0 |
| HiSeq1459 | P-11-0592 | P-11-0592-C1 | cfDNA | GCB | 2.968 | 44 | M | NA | 3 | 4 | 0 |
| HiSeq1459 | P-02-0054 | P-02-0054-C1 | cfDNA | Normal | NA | 66 | M | NA | NA | | 1 |
| HiSeq1459 | P-02-0050 | P-02-0050-C1 | cfDNA | Normal | NA | 77 | M | NA | NA | | 1 |
| HiSeq1459 | P-02-0004 | P-02-0004-C1 | cfDNA | Normal | NA | 72 | F | NA | NA | | 1 |
| HiSeq1459 | P-02-0029 | P-02-0029-C1 | cfDNA | Normal | NA | 72 | F | NA | NA | | 1 |
| HiSeq1459 | P-01-0160 | P-01-0160-C2 | cfDNA | Normal | NA | NA | M | NA | NA | | 1 |
| HiSeq1638 | P-14-0661 | P-14-0661-C1 | cfDNA | ABC | 0.326642 | 43 | M | NA | 3 | 4 | 0 |
| HiSeq1638 | P-14-0671 | P-14-0671-C1 | cfDNA | ABC | 4.53687 | 33 | M | NA | 2 | 4 | 0 |
| HiSeq1638 | P-14-0616 | P-14-0616-C1 | cfDNA | ABC | 12.10561 | 52 | F | NA | 4 | 4 | 0 |
| HiSeq1639 | P-14-0606 | P-14-0606-C1 | cfDNA | DLBCL | 1.042847 | 70 | F | NA | 3 | 3 | 0 |
| HiSeq1639 | P-14-0619 | P-14-0619-C1 | cfDNA | DLBCL | 2.036304 | 48 | M | NA | 2 | 4 | 0 |
| HiSeq1639 | P-14-0620 | P-14-0620-C1 | cfDNA | DLBCL | 3.963679 | 71 | F | NA | 3 | 3 | 0 |
| HiSeq1639 | P-14-0622 | P-14-0622-C1 | cfDNA | GCB | 10.75207 | 51 | M | NA | 3 | 4 | 0 |
| HiSeq1639 | P-14-0626 | P-14-0626-C1 | cfDNA | GCB | 2.943261 | 68 | F | NA | 3 | 4 | 0 |
| HiSeq1639 | P-14-0629 | P-14-0629-C1 | cfDNA | ABC | 0.411568 | 66 | M | NA | 2 | 3 | 0 |
| HiSeq1639 | P-14-0634 | P-14-0634-C1 | cfDNA | GCB | 4.674999 | 77 | F | NA | 4 | 4 | 0 |
| HiSeq1639 | P-14-0643 | P-14-0643-C1 | cfDNA | DLBCL | 9.53051 | 55 | M | NA | 2 | 4 | 0 |
| HiSeq1639 | P-14-0672 | P-14-0672-C1 | cfDNA | ABC | 8.277267 | NA | NA | NA | NA | NA | 0 |
| HiSeq1639 | P-14-0673 | P-14-0673-C1 | cfDNA | ABC | 0.942809 | NA | NA | NA | NA | NA | 0 |
| HiSeq1639 | P-14-0683 | P-14-0683-C1 | cfDNA | DLBCL | 7.527635 | 55 | F | NA | 3 | 4 | 0 |
| HiSeq1633 | P-20-0791 | P-20-0791-C1 | cfDNA | LUSC | 0 | 73 | M | NA | NA | IIA | 1 |
| HiSeq1633 | P-20-0599 | P-20-0599-C1 | cfDNA | LUSC | 0.0551 | 68 | M | NA | NA | IIIA | 1 |
| HiSeq1633 | P-20-0795 | P-20-0795-C1 | cfDNA | LUAD | 0 | 62 | F | NA | NA | IIIB | 1 |
| HiSeq1633 | P-20-0614 | P-20-0614-C1 | cfDNA | LUAD | 0.587 | 52 | M | NA | NA | IIIA | 1 |
| HiSeq1633 | P-20-0615 | P-20-0615-C1 | cfDNA | LUSC | 0 | 74 | M | NA | NA | IA | 1 |
| HiSeq1633 | P-20-0306 | P-20-0306-C1 | cfDNA | LUSC | 0 | 71 | F | NA | NA | IA | 1 |
| HiSeq1677 | P-20-0551 | P-20-0551-C1 | cfDNA | LUAD | 0 | 64 | F | NA | NA | IIIA | 1 |
| HiSeq1633 | P-20-0511 | P-20-0511-C1 | cfDNA | LUAD | 0 | 77 | M | NA | NA | IB | 1 |
| HiSeq1633 | P-20-0663 | P-20-0663-C1 | cfDNA | LUAD | 0.05 | 68 | M | NA | NA | IIB | 1 |
| HiSeq1633 | P-20-0776 | P-20-0776-C1 | cfDNA | LUAD | 1.0009 | 57 | F | NA | NA | IV | 1 |
| HiSeq1633 | P-02-0049 | P-02-0049-C1 | cfDNA | Normal | NA | 66 | M | NA | NA | | 1 |
| HiSeq1677 | P-02-0035 | P-02-0035-C1 | cfDNA | Normal | NA | 76 | M | NA | NA | | 1 |
| HiSeq1633 | P-02-0036 | P-02-0036-C1 | cfDNA | Normal | NA | 64 | F | NA | NA | | 1 |
| HiSeq1677 | P-20-0513 | P-20-0513-C1 | cfDNA | LUAD | 0 | 70 | M | NA | NA | IIA | 1 |
| HiSeq1634 | P-20-0675 | P-20-0675-C1 | cfDNA | LUAD | 0.527486 | 75 | F | NA | NA | IV | 1 |
| HiSeq1634 | P-20-0684 | P-20-0684-C2 | cfDNA | LUSC | 2.119759 | 84 | M | NA | NA | IV | 0 |
| HiSeq1634 | P-20-0692 | P-20-0692-C2 | cfDNA | LUAD | 0.994039 | 74 | F | NA | NA | IV | 1 |
| HiSeq1634 | P-20-0692 | P-20-0692-C3 | cfDNA | LUAD | 0.078847 | 74 | F | NA | NA | IV | 0 |
| HiSeq1634 | P-20-0675 | P-20-0675-C2 | cfDNA | LUAD | 0.06063 | 75 | F | NA | NA | IV | 0 |
| HiSeq1634 | P-20-0694 | P-20-0694-C2 | cfDNA | LUAD | 0.390546 | 58 | F | NA | NA | IV | 1 |
| HiSeq1634 | P-20-0694 | P-20-0694-C3 | cfDNA | LUAD | 0.067471 | 58 | F | NA | NA | IV | 0 |
| HiSeq1634 | P-20-0693 | P-20-0693-C1 | cfDNA | LUAD | 2.117064 | 72 | E | NA | NA | IV | 1 |
| HiSeq1634 | P-20-0693 | P-20-0693-C2 | cfDNA | LUAD | 0.502387 | 72 | F | NA | NA | IV | 0 |
| HiSeq1634 | P-20-0816 | P-20-0816-C1 | cfDNA | LUSC | 0.46653 | 70 | F | NA | NA | IV | 1 |
| HiSeq1634 | P-02-0010 | P-02-0010-C1 | cfDNA | Normal | NA | 62 | F | NA | NA | NA | 1 |
| HiSeq1634 | P-02-0012 | P-02-0012-C1 | cfDNA | Normal | NA | 66 | M | NA | NA | NA | 1 |
| HiSeq1634 | P-02-0031 | P-02-0031-C1 | cfDNA | Normal | NA | 72 | M | NA | NA | NA | 1 |
| HiSeq1635 | P-20-0671 | P-20-0671-C1 | cfDNA | LUAD | 0 | 68 | M | NA | NA | IIIA | 1 |
| HiSeq1635 | P-20-0476 | P-20-0476-C1 | cfDNA | LUSC | 0.016327 | 66 | M | NA | NA | IIB | 1 |
| HiSeq1635 | P-20-0782 | P-20-0782-C3 | cfDNA | LUAD | 2.12191 | 73 | F | NA | NA | IV | 1 |
| HiSeq1635 | P-20-0782 | P-20-0782-C4 | cfDNA | LUAD | 4.736844 | 73 | F | NA | NA | IV | 0 |
| HiSeq1635 | P-20-0780 | P-20-0780-C2 | cfDNA | LUAD | 2.579815 | 50 | M | NA | NA | IV | 0 |
| HiSeq1635 | P-20-0781 | P-20-0781-C2 | cfDNA | LUAD | 2.906975 | 58 | F | NA | NA | IV | 0 |
| HiSeq1635 | P-20-0695 | P-20-0695-C2 | cfDNA | LUAD | 0.713188 | 76 | M | NA | NA | IV | 0 |
| HiSeq1635 | P-20-0816 | P-20-0816-C2 | cfDNA | LUSC | 0.082797 | 70 | F | NA | NA | IV | 0 |
| HiSeq1635 | P-20-0780 | P-20-0780-C1 | cfDNA | LUAD | 0.908664 | 50 | M | NA | NA | IV | 1 |
| HiSeq1635 | P-02-0047 | P-02-0047-C1 | cfDNA | Normal | NA | 75 | M | NA | NA | NA | 1 |
| HiSeq1635 | P-02-0048 | P-02-0048-C1 | cfDNA | Normal | NA | 72 | M | NA | NA | NA | 1 |
| HiSeq1635 | P-02-0028 | P-02-0028-C1 | cfDNA | Normal | NA | 54 | M | NA | NA | NA | 1 |
| HiSeq1635 | P-02-0033 | P-02-0033-C1 | cfDNA | Normal | NA | 77 | M | NA | NA | NA | 1 |
| HiSeq1635 | P-14-0789 | P-14-0789-C1 | cfDNA | GCB | 5.840683 | 55 | M | NA | 0 | 2 | 0 |
| HiSeq1638 | P-14-0790 | P-14-0790-C1 | cfDNA | GCB | 0.94064 | 70 | M | NA | 2 | 2 | 0 |
| HiSeq1638 | P-14-0669 | P-14-0669-C1 | cfDNA | GCB | 10.67179 | NA | NA | NA | NA | NA | 0 |
| HiSeq1638 | P-14-0737 | P-14-0737-P05 | cfDNA | GCB | 6.255337 | 62 | M | NA | 3 | 4 | 0 |
| HiSeq1638 | P-14-0667 | P-14-0667-C1 | cfDNA | GCB | 2.015113 | NA | NA | NA | NA | NA | 0 |
| HiSeq1638 | P-14-0537 | P-14-0537-C0FIXED | cfDNA | ABC | 0.408595 | 59 | M | NA | 1 | 3 | 0 |
| HiSeq1638 | P-14-0746 | P-14-0746-C2 | cfDNA | ABC | 2.895452 | 30 | F | NA | 3 | 4 | 0 |
| HiSeq1638 | P-14-0741 | P-14-0741-P02 | cfDNA | ABC | 0.19089 | 60 | M | NA | 3 | 4 | 0 |
| HiSeq1638 | P-05-0002 | P-05-0002-C1 | cfDNA | Normal | NA | 40 | M | NA | NA | NA | 1 |
| HiSeq1638 | P-05-0003 | P-05-0003-C1 | cfDNA | Normal | NA | 38 | M | NA | NA | NA | 1 |
| HiSeq1639 | P-14-0793 | P-14-0793-C1 | cfDNA | GCB | 1.152029 | 78 | M | NA | 2 | 1 | 0 |
| HiSeq1639 | P-05-0001 | P-05-0001-C1 | cfDNA | Normal | NA | 35 | M | NA | NA | NA | 1 |
| HiSeq1639 | P-05-0004 | P-05-0004-C1 | cfDNA | Normal | NA | 38 | M | NA | NA | NA | 1 |
| HiSeq1674 | P-20-0776 | P-20-0776-C2 | cfDNA | LUAD | 1.201065 | 57 | F | NA | NA | IV | 0 |
| HiSeq1637 | P-02-0015 | P-02-0015-C1 | cfDNA | Normal | NA | 67 | M | NA | NA | NA | 1 |
| HiSeq1638 | P-02-0041 | P-02-0041-C1 | cfDNA | Normal | NA | 83 | M | NA | NA | NA | 1 |
| HiSeq1639 | P-02-0044 | P-02-0044-C1 | cfDNA | Normal | NA | 70 | M | NA | NA | NA | 1 |
| HiSeq1640 | P-02-0023 | P-02-0023-C1 | cfDNA | Normal | NA | 77 | M | NA | NA | NA | 1 |
| HiSeq1641 | P-02-0024 | P-02-0024-C1 | cfDNA | Normal | NA | 69 | F | NA | NA | NA | 1 |
| HiSeq1645 | P-01-0013 | P-01-0013-C5-EDTA | cfDNA | Normal | NA | 29 | M | NA | NA | | 1 |
| HiSeq1646 | P-01-0034 | P-01-0034-C3-EDTA | cfDNA | Normal | NA | 41 | M | NA | NA | | 1 |
| HiSeq1647 | P-01-0073 | P-01-0073-C2-EDTA | cfDNA | Normal | NA | 22 | M | NA | NA | | 1 |
| HiSeq1649 | P-01-0029 | P-01-0029-C3-EDTA | cfDNA | Normal | NA | 45 | M | NA | NA | 3 | 1 |

| | | | | | | DLBCL | EPIC- | NSCLC | EPIC- | DLBCL- |
|---|---|---|---|---|---|---|---|---|---|---|
| | NSCLC | EPIC- | DLBCL | NSCLC | | Time | lung- | histology- | lymphoma- | COO- |
| RunName | histology | lymphoma | COO | ICI | cohort | Series | score | score | score | score |
| HiSeq1004 | 1 | 0 | 0 | 0 | Discovery/CV | 0 | 0.984632 | 0.995579 | NA | NA |
| HiSeq1004 | 1 | 0 | 0 | 0 | Discovery/CV | 0 | 0.976948 | 0.526302 | NA | NA |
| HiSeq1004 | 1 | 0 | 0 | 0 | Discovery/CV | 0 | 0.739243 | 0.031334 | NA | NA |
| HiSeq1005 | 1 | 0 | 0 | 0 | Discovery/CV | 0 | 0.999672 | 0.071847 | NA | NA |
| HiSeq1005 | 1 | 0 | 0 | 0 | Discovery/CV | 0 | 0.832555 | 0.417164 | NA | NA |
| HiSeq1005 | 1 | 0 | 0 | 0 | Discovery/CV | 0 | 0.61709 | 0.081372 | NA | NA |
| HiSeq1327 | 1 | 0 | 0 | 0 | Discovery/CV | 0 | 0.998332 | 0.78443 | NA | NA |
| HiSeq1005 | 1 | 0 | 0 | 0 | Discovery/CV | 0 | 0.99944 | 0.000734 | NA | NA |
| HiSeq1005 | 1 | 0 | 0 | 0 | Discovery/CV | 0 | 0.99988 | 0.996235 | NA | NA |
| HiSeq1006 | 1 | 0 | 0 | 0 | Discovery/CV | 0 | 0.999974 | 0.077853 | NA | NA |
| HiSeq1006 | 1 | 0 | 0 | 0 | Discovery/CV | 0 | 0.996846 | 0.946425 | NA | NA |
| HiSeq1006 | 1 | 0 | 0 | 0 | Discovery/CV | 0 | 0.855203 | 0.241835 | NA | NA |
| HiSeq1006 | 1 | 0 | 0 | 0 | Discovery/CV | 0 | 0.616546 | 0.014827 | NA | NA |
| HiSeq1114 | 1 | 0 | 0 | 1 | Discovery/CV | 0 | 0.999545 | 0.98048 | NA | NA |
| HiSeq1113 | 0 | 0 | 0 | 1 | Discovery/CV | 0 | NA | NA | NA | NA |
| HiSeq1113 | 0 | 0 | 0 | 1 | Discovery/CV | 0 | NA | NA | NA | NA |
| HiSeq1113 | 1 | 0 | 0 | 1 | Discovery/CV | 0 | 0.999957 | 0.992598 | NA | NA |
| HiSeq1113 | 0 | 0 | 0 | 1 | Discovery/CV | 0 | NA | NA | NA | NA |
| HiSeq1113 | 1 | 0 | 0 | 1 | Discovery/CV | 0 | 0.104327 | 0.768856 | NA | NA |
| HiSeq1113 | 1 | 0 | 0 | 1 | Discovery/CV | 0 | 0.47896 | 0.994418 | NA | NA |
| HiSeq1113 | 0 | 0 | 0 | 1 | Discovery/CV | 0 | NA | NA | NA | NA |
| HiSeq1114 | 1 | 0 | 0 | 1 | Discovery/CV | 0 | 0.997776 | 0.999999 | NA | NA |
| HiSeq1327 | 0 | 0 | 0 | 0 | Discovery/CV | 0 | NA | NA | NA | NA |
| HiSeq1114 | 1 | 0 | 0 | 1 | Discovery/CV | 0 | 0.908072 | 0.999633 | NA | NA |
| HiSeq1114 | 0 | 0 | 0 | 1 | Discovery/CV | 0 | NA | NA | NA | NA |
| HiSeq1114 | 0 | 0 | 0 | 1 | Discovery/CV | 0 | NA | NA | NA | NA |
| HiSeq1144 | 0 | 0 | 0 | 0 | Discovery/CV | 0 | NA | NA | NA | NA |
| HiSeq1144 | 0 | 1 | 1 | 0 | Discovery/CV | 0 | NA | NA | 0.995053 | −0.03469 |
| HiSeq1144 | 0 | 1 | 1 | 0 | Discovery/CV | 0 | NA | NA | 0.906691 | −0.15442 |
| HiSeq1144 | 0 | 1 | 1 | 0 | Discovery/CV | 0 | NA | NA | 0.871968 | 0.063774 |
| HiSeq1144 | 0 | 1 | 1 | 0 | Discovery/CV | 0 | NA | NA | 0.999419 | 0.244844 |
| HiSeq1144 | 0 | 1 | 1 | 0 | Discovery/CV | 0 | NA | NA | 0.99997 | 0.431026 |
| HiSeq1145 | 0 | 1 | 1 | 0 | Discovery/CV | 0 | NA | NA | 0.85174 | −0.01484 |
| HiSeq1145 | 0 | 1 | 1 | 0 | Discovery/CV | 0 | NA | NA | 0.996151 | −0.17534 |
| HiSeq1145 | 0 | 1 | 1 | 0 | Discovery/CV | 0 | NA | NA | 0.998722 | 0.222533 |
| HiSeq1145 | 0 | 1 | 1 | 0 | Discovery/CV | 0 | NA | NA | 0.999933 | 0.316786 |
| HiSeq1145 | 0 | 1 | 1 | 0 | Discovery/CV | 0 | NA | NA | 0.995078 | 0.160599 |
| HiSeq1145 | 0 | 1 | 1 | 0 | Discovery/CV | 0 | NA | NA | 0.999565 | −0.29653 |
| HiSeq1146 | 0 | 1 | 1 | 0 | Discovery/CV | 0 | NA | NA | 1 | 0.209116 |
| HiSeq1146 | 0 | 1 | 1 | 0 | Discovery/CV | 0 | NA | NA | 2.24E−05 | 0.436934 |
| HiSeq1146 | | 1 | 1 | 0 | Discovery/CV | 0 | NA | NA | 0.694466 | 0.065037 |
| HiSeq1146 | 0 | 1 | 1 | 0 | Discovery/CV | 0 | NA | NA | 0.998894 | 0.093574 |
| HiSeq1146 | 0 | 0 | 0 | 0 | Discovery/CV | 0 | NA | NA | NA | NA |
| HiSeq1146 | 0 | 1 | 1 | 0 | Discovery/CV | 0 | NA | NA | 0.9995 | −0.55784 |
| HiSeq1146 | 0 | 1 | 1 | 0 | Discovery/CV | 0 | NA | NA | 0.999999 | 0.459411 |
| HiSeq1146 | 0 | 1 | 1 | 0 | Discovery/CV | 0 | NA | NA | 0.997397 | 0.31843 |
| HiSeq449 | 1 | 0 | 0 | 0 | Discovery/CV | 0 | 0.995173 | 0.999243 | NA | NA |
| HiSeq449 | 1 | 0 | 0 | 0 | Discovery/CV | 0 | 0.950931 | 0.926282 | NA | NA |
| HiSeq449 | 1 | 0 | 0 | 0 | Discovery/CV | 0 | 0.089664 | 1 | NA | NA |
| HiSeq449 | 0 | 1 | 1 | 0 | Discovery/CV | 0 | NA | NA | 1 | −0.03582 |
| HiSeq449 | 0 | 1 | 1 | 0 | Discovery/CV | 0 | NA | NA | 0.999993 | 0.285744 |
| HiSeq449 | 0 | 1 | 1 | 0 | Discovery/CV | 0 | NA | NA | 0.991072 | −0.27251 |
| HiSeq449 | 0 | 0 | 0 | 0 | Discovery/CV | 0 | NA | NA | NA | NA |
| HiSeq449 | 1 | 0 | 0 | 0 | Discovery/CV | 0 | 0.364178 | 0.000205 | NA | NA |
| HiSeq449 | 1 | 0 | 0 | 0 | Discovery/CV | 0 | 0.974247 | 0.002024 | NA | NA |
| HiSeq449 | 1 | 0 | 0 | 0 | Discovery/CV | 0 | 0.655499 | 0.365605 | NA | NA |
| HiSeq449 | 0 | 0 | 0 | 0 | Discovery/CV | 0 | NA | NA | NA | NA |
| HiSeq449 | 0 | 1 | 1 | 0 | Discovery/CV | 0 | NA | NA | 0.999968 | −0.32483 |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq508 | 0 | 0 | 0 | 0 | Discovery/CV | 0 | NA | NA | NA | NA |
| HiSeq618 | 1 | 0 | 0 | 0 | Discovery/CV | 0 | 0.849889 | 0.035002 | NA | NA |
| HiSeq618 | 1 | 0 | 0 | 0 | Discovery/CV | 0 | 0.980779 | 0.871275 | NA | NA |
| HiSeq618 | 1 | 0 | 0 | 0 | Discovery/CV | 0 | 0.991582 | 0.117303 | NA | NA |
| HiSeq618 | 0 | 1 | 1 | 0 | Discovery/CV | 0 | NA | NA | 0.999455 | −0.47348 |
| HiSeq618 | 0 | 0 | 0 | 0 | Discovery/CV | 1 | NA | NA | NA | NA |
| HiSeq618 | 1 | 0 | 0 | 0 | Discovery/CV | 0 | 0.423299 | 0.295154 | NA | NA |
| HiSeq618 | 1 | 0 | 0 | 0 | Discovery/CV | 0 | 0.959207 | 0.357672 | NA | NA |
| HiSeq618 | 1 | 0 | 0 | 0 | Discovery/CV | 0 | 0.999362 | 0.043994 | NA | NA |
| HiSeq620 | 0 | 0 | 0 | 0 | Discovery/CV | 0 | NA | NA | NA | NA |
| HiSeq620 | 0 | 0 | 0 | 0 | Discovery/CV | 0 | NA | NA | NA | NA |
| HiSeq620 | 0 | 0 | 0 | 0 | Discovery/CV | 0 | NA | NA | NA | NA |
| HiSeq620 | 0 | 1 | 1 | 0 | Discovery/CV | 0 | NA | NA | 1 | 0.231208 |
| HiSeq620 | 0 | 0 | 0 | 0 | Discovery/CV | 0 | NA | NA | NA | NA |
| HiSeq620 | 0 | 0 | 0 | 0 | Discovery/CV | 0 | NA | NA | NA | NA |
| HiSeq620 | 0 | 0 | 0 | 0 | Discovery/CV | 0 | NA | NA | NA | NA |
| HiSeq620 | 0 | 0 | 0 | 0 | Discovery/CV | 0 | NA | NA | NA | NA |
| HiSeq623 | 0 | 1 | 1 | 0 | Discovery/CV | 0 | NA | NA | 0.996081 | −0.41978 |
| HiSeq623 | 0 | 1 | 1 | 0 | Discovery/CV | 0 | NA | NA | 0.999865 | −0.37693 |
| HiSeq623 | 0 | 1 | 1 | 0 | Discovery/CV | 0 | NA | NA | 0.956331 | −0.19298 |
| HiSeq623 | 0 | 1 | 1 | 0 | Discovery/CV | 0 | NA | NA | 1 | 0.175532 |
| HiSeq623 | 0 | 1 | 1 | 0 | Discovery/CV | 0 | NA | NA | 1 | 0.414202 |
| HiSeq623 | 0 | 1 | 1 | 0 | Discovery/CV | 0 | NA | NA | 0.999995 | −0.35482 |
| HiSeq623 | 0 | 1 | 1 | 0 | Discovery/CV | 0 | NA | NA | 0.999983 | −0.4725 |
| HiSeq623 | 0 | 1 | 1 | 0 | Discovery/CV | 0 | NA | NA | 1 | −0.0652 |
| HiSeq623 | 0 | 1 | 1 | 0 | Discovery/CV | 0 | NA | NA | 0.999999 | 0.249195 |
| HiSeq623 | 0 | 1 | 1 | 0 | Discovery/CV | 0 | NA | NA | 0.989611 | 0.14254 |
| HiSeq624 | 1 | 0 | 0 | 0 | Discovery/CV | 0 | 0.992292 | 0.998884 | NA | NA |
| HiSeq624 | 0 | 1 | 1 | 0 | Discovery/CV | 0 | NA | NA | 0.547489 | 0.190244 |
| HiSeq624 | 1 | 0 | 0 | 0 | Discovery/CV | 0 | 0.68232 | 0.267728 | NA | NA |
| HiSeq724 | 0 | 1 | 0 | 0 | Discovery/CV | 0 | 0.002507 | NA | 0.337616 | NA |
| HiSeq724 | 0 | 1 | 0 | 0 | Discovery/CV | 0 | 0.040109 | NA | 0.201685 | NA |
| HiSeq724 | 0 | 1 | 0 | 0 | Discovery/CV | 0 | 0.025026 | NA | 0.156252 | NA |
| HiSeq724 | 0 | 1 | 0 | 0 | Discovery/CV | 0 | 0.254171 | NA | 0.590672 | NA |
| HiSeq724 | 0 | 1 | 0 | 0 | Discovery/CV | 0 | 0.074681 | NA | 0.216127 | NA |
| HiSeq724 | 0 | 1 | 0 | 0 | Discovery/CV | 0 | 0.017399 | NA | 0.20582 | NA |
| HiSeq724 | 0 | 1 | 0 | 0 | Discovery/CV | 0 | 0.008277 | NA | 0.319764 | NA |
| HiSeq724 | 0 | 1 | 0 | 0 | Discovery/CV | 0 | 0.189931 | NA | 0.097302 | NA |
| HiSeq724 | 0 | 1 | 0 | 0 | Discovery/CV | 0 | 0.003147 | NA | 0.13391 | NA |
| HiSeq724 | 0 | 1 | 0 | 0 | Discovery/CV | 0 | 0.003266 | NA | 0.16752 | NA |
| HiSeq724 | 0 | 1 | 0 | 0 | Discovery/CV | 0 | 0.19011 | NA | 0.210673 | NA |
| HiSeq724 | 0 | 1 | 0 | 0 | Discovery/CV | 0 | 0.036433 | NA | 0.281205 | NA |
| HiSeq804 | 1 | 0 | 0 | 0 | Discovery/CV | 0 | 0.573185 | 0.002282 | NA | NA |
| HiSeq804 | 1 | 0 | 0 | 0 | Discovery/CV | 0 | 0.658108 | 0.352247 | NA | NA |
| HiSeq804 | 0 | 0 | 0 | 0 | Discovery/CV | 0 | NA | NA | NA | NA |
| HiSeq804 | 1 | 0 | 0 | 0 | Discovery/CV | 0 | 0.956751 | 0.522954 | NA | NA |
| HiSeq804 | 1 | 0 | 0 | 0 | Discovery/CV | 0 | 0.70421 | 0.167 | NA | NA |
| HiSeq804 | 1 | 0 | 0 | 0 | Discovery/CV | 0 | 0.296821 | 0.393434 | NA | NA |
| HiSeq841 | 1 | 0 | 0 | 0 | Discovery/CV | 0 | 0.141199 | 0.451685 | NA | NA |
| HiSeq841 | 0 | 1 | 1 | 0 | Discovery/CV | 0 | NA | NA | 0.997524 | −0.01835 |
| HiSeq841 | 0 | 1 | 1 | 0 | Discovery/CV | 0 | NA | NA | 0.526583 | 0.030386 |
| HiSeq841 | 0 | 1 | 1 | 0 | Discovery/CV | 0 | NA | NA | 0.381999 | 0.050754 |
| HiSeq841 | 0 | 1 | 1 | 0 | Discovery/CV | 0 | NA | NA | 0.999807 | −0.26466 |
| HiSeq841 | 0 | 1 | 1 | 0 | Discovery/CV | 0 | NA | NA | 0.948631 | 0.204907 |
| HiSeq841 | 0 | 1 | 1 | 0 | Discovery/CV | 0 | NA | NA | 0.772132 | 0.010741 |
| HiSeq841 | 0 | 1 | 1 | 0 | Discovery/CV | 0 | NA | NA | 0.567087 | 0.167485 |
| HiSeq841 | 0 | 0 | 0 | 0 | Discovery/CV | 0 | NA | NA | NA | NA |
| HiSeq856 | 1 | 0 | 0 | 0 | Discovery/CV | 0 | 0.999984 | 0.658818 | NA | NA |
| HiSeq856 | 0 | 1 | 0 | 0 | Discovery/CV | 0 | 0.538636 | NA | 0.516641 | NA |
| HiSeq856 | 0 | 1 | 1 | 0 | Discovery/CV | 0 | NA | NA | 0.985333 | 0.083669 |
| HiSeq856 | 1 | 0 | 0 | 0 | Discovery/CV | 0 | 0.9025 | 0.195737 | NA | NA |
| HiSeq856 | 0 | 1 | 1 | 0 | Discovery/CV | 0 | NA | NA | 0.980188 | 0.047878 |
| HiSeq856 | 0 | 1 | 1 | 0 | Discovery/CV | 0 | NA | NA | 0.309405 | −0.0019 |
| HiSeq1327 | 1 | 0 | 0 | 0 | Discovery/CV | 0 | 0.960535 | 0.789133 | NA | NA |
| HiSeq1327 | 1 | 0 | 0 | 0 | Discovery/CV | 0 | 0.207486 | 0.246602 | NA | NA |
| HiSeq1327 | 1 | 0 | 0 | 0 | Discovery/CV | 0 | 0.011732 | 0.579459 | NA | NA |
| HiSeq1327 | 1 | 0 | 0 | 0 | Discovery/CV | 0 | 0.057501 | 0.223909 | NA | NA |
| HiSeq1326 | 0 | 1 | 0 | 0 | Discovery/CV | 0 | 0.127518 | NA | 0.083592 | NA |
| HiSeq1326 | 0 | 1 | 0 | 0 | Discovery/CV | 0 | 0.000642 | NA | 0.051161 | NA |
| HiSeq1326 | 0 | 1 | 0 | 0 | Discovery/CV | 0 | 0.061126 | NA | 0.040506 | NA |
| HiSeq1326 | 0 | 1 | 0 | 0 | Discovery/CV | 0 | 0.124073 | NA | 0.013732 | NA |
| HiSeq1326 | 0 | 1 | 0 | 0 | Discovery/CV | 0 | 0.038439 | NA | 0.010096 | NA |
| HiSeq1326 | 0 | 1 | 0 | 0 | Discovery/CV | 0 | 0.003837 | NA | 0.062397 | NA |
| HiSeq1326 | 0 | 1 | 0 | 0 | Discovery/CV | 0 | 0.101902 | NA | 0.050371 | NA |
| HiSeq1326 | 0 | 1 | 0 | 0 | Discovery/CV | 0 | 0.000519 | NA | 0.062453 | NA |
| HiSeq1326 | 0 | 1 | 0 | 0 | Discovery/CV | 0 | 0.1395 | NA | 0.018041 | NA |
| HiSeq1326 | 0 | 1 | 0 | 0 | Discovery/CV | 0 | 0.393569 | NA | 0.088995 | NA |
| HiSeq1326 | 0 | 1 | 0 | 0 | Discovery/CV | 0 | 0.315205 | NA | 0.987041 | NA |
| HiSeq1326 | 0 | 1 | 0 | 0 | Discovery/CV | 0 | 0.001215 | NA | 0.000576 | NA |
| HiSeq1327 | 0 | 1 | 0 | 0 | Discovery/CV | 0 | 0.001598 | NA | 0.051213 | NA |
| HiSeq1326 | 0 | 1 | 0 | 0 | Discovery/CV | 0 | 0.360864 | NA | 0.150183 | NA |
| HiSeq1327 | 0 | 1 | 0 | 0 | Discovery/CV | 0 | 0.000827 | NA | 0.048588 | NA |
| HiSeq1326 | 0 | 1 | 0 | 0 | Discovery/CV | 0 | 0.061583 | NA | 0.20282 | NA |
| HiSeq1327 | 0 | 1 | 0 | 0 | Discovery/CV | 0 | 0.030144 | NA | 0.166197 | NA |
| HiSeq1327 | 0 | 1 | 0 | 0 | Discovery/CV | 0 | 0.000529 | NA | 0.185023 | NA |
| HiSeq1327 | 0 | 1 | 0 | 0 | Discovery/CV | 0 | 0.241909 | NA | 0.275842 | NA |
| HiSeq1327 | 0 | 1 | 0 | 0 | Discovery/CV | 0 | 0.102845 | NA | 0.122756 | NA |
| HiSeq1327 | 0 | 1 | 0 | 0 | Discovery/CV | 0 | 0.542633 | NA | 0.154844 | NA |
| HiSeq1327 | 0 | 1 | 0 | 0 | Discovery/CV | 0 | 0.793859 | NA | 0.047349 | NA |
| HiSeq1363 | 0 | 0 | 0 | 0 | Discovery/CV | 0 | 0.635714 | 0.919271 | NA | NA |
| HiSeq1363 | 0 | 0 | 0 | 0 | Discovery/CV | 0 | 0.77444 | 0.61389 | NA | NA |
| HiSeq1363 | 0 | 0 | 0 | 0 | Discovery/CV | 0 | 0.871013 | 0.178213 | NA | NA |
| HiSeq1363 | 1 | 0 | 0 | 0 | Discovery/CV | 0 | 0.988316 | 1.17E−06 | NA | NA |
| HiSeq1363 | 1 | 0 | 0 | 0 | Discovery/CV | 0 | 0.115152 | 0.45496 | NA | NA |
| HiSeq1363 | 1 | 0 | 0 | 0 | Discovery/CV | 0 | 0.634155 | 0.574253 | NA | NA |
| HiSeq1363 | 1 | 0 | 0 | 0 | Discovery/CV | 0 | 0.866278 | 0.481296 | NA | NA |
| HiSeq1363 | 1 | 0 | 0 | 0 | Discovery/CV | 0 | 0.060457 | 0.735928 | NA | NA |
| HiSeq1363 | 1 | 0 | 0 | 0 | Discovery/CV | 0 | 0.999952 | 0.98311 | NA | NA |
| HiSeq1364 | 1 | 0 | 0 | 0 | Discovery/CV | 0 | 0.986572 | 0.394612 | NA | NA |
| HiSeq1364 | 1 | 0 | 0 | 0 | Discovery/CV | 0 | 0.992259 | 0.521838 | NA | NA |
| HiSeq1364 | 1 | 0 | 0 | 0 | Discovery/CV | 0 | 0.93404 | 0.864854 | NA | NA |
| HiSeq1364 | 1 | 0 | 0 | 0 | Discovery/CV | 0 | 0.861158 | 0.004031 | NA | NA |
| HiSeq1364 | 1 | 0 | 0 | 0 | Discovery/CV | 0 | 0.997359 | 0.730219 | NA | NA |
| HiSeq1364 | 1 | 0 | 0 | 0 | Discovery/CV | 0 | 0.982671 | 0.857206 | NA | NA |
| HiSeq1364 | 1 | 0 | 0 | 0 | Discovery/CV | 0 | 0.981635 | 0.555881 | NA | NA |
| HiSeq1364 | 1 | 0 | 0 | 0 | Discovery/CV | 0 | 0.663797 | 0.005636 | NA | NA |
| HiSeq1363 | 0 | 1 | 0 | 0 | Discovery/CV | 0 | 0.005385 | NA | 0.585721 | NA |
| HiSeq1363 | 0 | 1 | 0 | 0 | Discovery/CV | 0 | 0.01734 | NA | 0.100901 | NA |
| HiSeq1363 | 0 | 1 | 0 | 0 | Discovery/CV | 0 | 0.013676 | NA | 0.104732 | NA |
| HiSeq1363 | 0 | 0 | 0 | 0 | Discovery/CV | 0 | NA | NA | NA | NA |
| HiSeq1363 | 0 | 1 | 0 | 0 | Discovery/CV | 0 | 0.028123 | NA | 0.857263 | NA |
| HiSeq1364 | 0 | 1 | 0 | 0 | Discovery/CV | 0 | 0.017105 | NA | 0.000772 | NA |
| HiSeq1364 | 0 | 1 | 0 | 0 | Discovery/CV | 0 | 0.154593 | NA | 0.103625 | NA |
| HiSeq1364 | 0 | 0 | 0 | 0 | Discovery/CV | 0 | NA | NA | NA | NA |
| HiSeq1364 | 0 | 1 | 0 | 0 | Discovery/CV | 0 | 0.011532 | NA | 0.006805 | NA |
| HiSeq1364 | 0 | 1 | 0 | 0 | Discovery/CV | 0 | 0.025769 | NA | 0.077912 | NA |
| HiSeq1357 | 0 | 1 | 1 | 0 | Discovery/CV | 0 | NA | NA | 0.948411 | 0.119932 |
| HiSeq1357 | 0 | 1 | 1 | 0 | Discovery/CV | 0 | NA | NA | 0.910783 | 0.097158 |
| HiSeq1357 | 0 | 1 | 1 | 0 | Discovery/CV | 0 | NA | NA | 0.253935 | 0.085908 |
| HiSeq1357 | 0 | 1 | 1 | 0 | Discovery/CV | 0 | NA | NA | 0.92503 | −0.11196 |
| HiSeq1357 | 0 | 1 | 1 | 0 | Discovery/CV | 0 | NA | NA | 0.363422 | 0.149462 |
| HiSeq1357 | 0 | 1 | 1 | 0 | Discovery/CV | 0 | NA | NA | 0.973083 | −0.08025 |
| HiSeq1357 | 0 | 1 | 1 | 0 | Discovery/CV | 0 | NA | NA | 0.673764 | 0.081151 |
| HiSeq1357 | 0 | 1 | 1 | 0 | Discovery/CV | 0 | NA | NA | 0.384435 | −0.01315 |
| HiSeq1357 | 0 | 1 | 0 | 0 | Discovery/CV | 0 | 0.954081 | NA | 0.560849 | NA |
| HiSeq1357 | 0 | 1 | 0 | 0 | Discovery/CV | 0 | 0.005167 | NA | 0.06763 | NA |
| HiSeq1357 | 0 | 1 | 0 | 0 | Discovery/CV | 0 | 0.661971 | NA | 0.190027 | NA |
| HiSeq1358 | 0 | 1 | 0 | 0 | Discovery/CV | 0 | 0.028843 | NA | 0.248533 | NA |
| HiSeq1358 | 0 | 1 | 1 | 0 | Discovery/CV | 0 | NA | NA | 0.999996 | −0.38025 |
| HiSeq1358 | 0 | 1 | 1 | 0 | Discovery/CV | 0 | NA | NA | 0.991392 | −0.34694 |
| HiSeq1358 | 0 | 1 | 1 | 0 | Discovery/CV | 0 | NA | NA | 0.999308 | −0.14039 |
| HiSeq1358 | 0 | 1 | 1 | 0 | Discovery/CV | 0 | NA | NA | 0.396276 | 0.080494 |
| HiSeq1358 | 0 | 1 | 1 | 0 | Discovery/CV | 0 | NA | NA | 0.99773 | −0.01634 |
| HiSeq1358 | 0 | 1 | 1 | 0 | Discovery/CV | 0 | NA | NA | 0.999989 | 0.120978 |
| HiSeq1358 | 0 | 1 | 0 | 0 | Discovery/CV | 0 | 0.374699 | NA | 0.098491 | NA |
| HiSeq1358 | 0 | 1 | 1 | 0 | Discovery/CV | 0 | NA | NA | 0.789082 | −0.04678 |
| HiSeq1359 | 0 | 1 | 0 | 0 | Discovery/CV | 0 | 0.018276 | NA | 7.62E−07 | NA |
| HiSeq1359 | 0 | 1 | 1 | 0 | Discovery/CV | 0 | NA | NA | 0.130562 | 0.173971 |
| HiSeq1359 | 0 | 1 | 1 | 0 | Discovery/CV | 0 | NA | NA | 0.988584 | −0.01911 |
| HiSeq1359 | 0 | 1 | 0 | 0 | Discovery/CV | 0 | 0.01134 | NA | 1.58E−05 | NA |
| HiSeq1359 | 0 | 1 | 1 | 0 | Discovery/CV | 0 | NA | NA | 1 | 0.097844 |
| HiSeq1359 | 0 | 1 | 1 | 0 | Discovery/CV | 0 | NA | NA | 0.997983 | 0.187171 |
| HiSeq1360 | 0 | 1 | 0 | 0 | Discovery/CV | 0 | 0.499353 | NA | 0.190683 | NA |
| HiSeq1360 | 0 | 1 | 0 | 0 | Discovery/CV | 0 | 0.246983 | NA | 0.852154 | NA |
| HiSeq1360 | 0 | 1 | 0 | 0 | Discovery/CV | 0 | 0.149161 | NA | 0.405563 | NA |
| HiSeq1360 | 0 | 1 | 0 | 0 | Discovery/CV | 0 | 0.012228 | NA | 0.004998 | NA |
| HiSeq1360 | 0 | 1 | 1 | 0 | Discovery/CV | 0 | NA | NA | 0.999988 | −0.66956 |
| HiSeq1360 | 0 | 1 | 1 | 0 | Discovery/CV | 0 | NA | NA | 0.941101 | 0.008131 |
| HiSeq1360 | 0 | 1 | 1 | 0 | Discovery/CV | 0 | NA | NA | 0.999971 | −0.02453 |
| HiSeq1360 | 0 | 1 | 1 | 0 | Discovery/CV | 0 | NA | NA | 0.988789 | 0.131243 |
| HiSeq1360 | 0 | 1 | 1 | 0 | Discovery/CV | 0 | NA | NA | 0.998986 | 0.026169 |
| HiSeq1360 | 0 | 1 | 1 | 0 | Discovery/CV | 0 | NA | NA | 0.896569 | 0.092816 |
| HiSeq1203 | 0 | 1 | 1 | 0 | Discovery/CV | 0 | NA | NA | 0.011085 | 0.154285 |
| HiSeq1203 | 0 | 1 | 1 | 0 | Discovery/CV | 0 | NA | NA | 0.064354 | 0.063592 |
| HiSeq1203 | 0 | 1 | 1 | 0 | Discovery/CV | 0 | NA | NA | 0.63415 | 0.058082 |
| HiSeq1203 | 0 | 1 | 1 | 0 | Discovery/CV | 0 | NA | NA | 0.008352 | 0.161489 |
| HiSeq1440 | 0 | 0 | 0 | 0 | Discovery/CV | 1 | NA | NA | NA | NA |
| HiSeq1440 | 0 | 0 | 0 | 0 | Discovery/CV | 1 | NA | NA | NA | NA |
| HiSeq1440 | 0 | 0 | 0 | 0 | Discovery/CV | 1 | NA | NA | NA | NA |
| HiSeq1440 | 0 | 0 | 0 | 0 | Discovery/CV | 1 | NA | NA | NA | NA |
| HiSeq1440 | 0 | 0 | 0 | 0 | Discovery/CV | 1 | NA | NA | NA | NA |
| HiSeq1440 | 0 | 0 | 0 | 0 | Discovery/CV | 1 | NA | NA | NA | NA |
| HiSeq1440 | 0 | 0 | 0 | 0 | Discovery/CV | 1 | NA | NA | NA | NA |
| HiSeq1440 | 0 | 0 | 0 | 0 | Discovery/CV | 1 | NA | NA | NA | NA |
| HiSeq1440 | 0 | 0 | 0 | 0 | Discovery/CV | 1 | NA | NA | NA | NA |
| HiSeq1440 | 0 | 0 | 0 | 0 | Discovery/CV | 1 | NA | NA | NA | NA |
| HiSeq1440 | 0 | 0 | 0 | 0 | Discovery/CV | 1 | NA | NA | NA | NA |
| HiSeq1440 | 0 | 1 | 0 | 0 | Discovery/CV | 0 | 0.162177 | NA | 0.114332 | NA |
| HiSeq1440 | 0 | 1 | 0 | 0 | Discovery/CV | 0 | 0.009718 | NA | 0.104793 | NA |
| HiSeq1440 | 0 | 0 | 0 | 0 | Discovery/CV | 0 | NA | NA | NA | NA |
| HiSeq1444 | 0 | 1 | 0 | 0 | Discovery/CV | 0 | 0.367517 | NA | 0.143958 | NA |
| HiSeq1444 | 0 | 1 | 0 | 0 | Discovery/CV | 0 | 0.14325 | NA | 0.117974 | NA |
| HiSeq1444 | 0 | 1 | 0 | 0 | Discovery/CV | 0 | 0.575583 | NA | 0.658445 | NA |
| HiSeq1444 | 0 | 1 | 0 | 0 | Discovery/CV | 0 | 0.398878 | NA | 0.173343 | NA |
| HiSeq1444 | 0 | 1 | 0 | 0 | Discovery/CV | 0 | 0.157554 | NA | 0.192513 | NA |
| HiSeq1444 | 0 | 1 | 0 | 0 | Discovery/CV | 0 | 0.179547 | NA | 0.088615 | NA |
| HiSeq1444 | 0 | 0 | 0 | 1 | Discovery/CV | 0 | NA | NA | NA | NA |
| HiSeq1444 | 1 | 0 | 0 | 1 | Discovery/CV | 0 | 0.790963 | 0.852884 | NA | NA |
| HiSeq1444 | 1 | 0 | 0 | 1 | Discovery/CV | 0 | 0.998814 | 0.827383 | NA | NA |
| HiSeq1444 | 1 | 0 | 0 | 1 | Discovery/CV | 0 | 0.361466 | 0.400453 | NA | NA |
| HiSeq1444 | 0 | 0 | 0 | 1 | Discovery/CV | 0 | NA | NA | NA | NA |
| HiSeq1444 | 0 | 0 | 0 | 1 | Discovery/CV | 0 | NA | NA | NA | NA |
| HiSeq1444 | 1 | 0 | 0 | 1 | Discovery/CV | 0 | 0.999972 | 0.785967 | NA | NA |
| HiSeq1444 | 0 | 0 | 0 | 1 | Discovery/CV | 0 | NA | NA | NA | NA |
| HiSeq1444 | 1 | 0 | 0 | 1 | Discovery/CV | 0 | 0.552766 | 0.992895 | NA | NA |
| HiSeq1444 | 0 | 0 | 0 | 1 | Discovery/CV | 0 | NA | NA | NA | NA |
| HiSeq1458 | 0 | 1 | 1 | 0 | Discovery/CV | 0 | NA | NA | 0.994963 | −0.13517 |
| HiSeq1458 | 0 | 1 | 1 | 0 | Discovery/CV | 0 | NA | NA | 0.957594 | −0.06531 |
| HiSeq1458 | 0 | 1 | 1 | 0 | Discovery/CV | 0 | NA | NA | 0.921332 | 0.28573 |
| HiSeq1458 | 0 | 1 | 1 | 0 | Discovery/CV | 0 | NA | NA | 0.881314 | −0.00095 |
| HiSeq1458 | 0 | 1 | 1 | 0 | Discovery/CV | 0 | NA | NA | 0.908873 | −0.07171 |
| HiSeq1458 | 0 | 1 | 1 | 0 | Discovery/CV | 0 | NA | NA | 0.761465 | 0.053799 |
| HiSeq1458 | 0 | 0 | 0 | 0 | Discovery/CV | 0 | NA | NA | NA | NA |
| HiSeq1458 | 0 | 1 | 1 | 0 | Discovery/CV | 0 | NA | NA | 0.999851 | −0.1228 |
| HiSeq1458 | 0 | 1 | 1 | 0 | Discovery/CV | 0 | NA | NA | 0.999622 | −0.09959 |
| HiSeq1458 | 0 | 0 | 0 | 0 | Discovery/CV | 0 | NA | NA | NA | NA |
| HiSeq1458 | 0 | 1 | 0 | 0 | Discovery/CV | 0 | 0.79586 | NA | 0.104143 | NA |
| HiSeq1458 | 0 | 1 | 0 | 0 | Discovery/CV | 0 | 0.671181 | NA | 0.151915 | NA |
| HiSeq1458 | 0 | 1 | 0 | 0 | Discovery/CV | 0 | 0.11572 | NA | 0.13456 | NA |
| HiSeq1458 | 0 | 1 | 0 | 0 | Discovery/CV | 0 | 0.216761 | NA | 0.387261 | NA |
| HiSeq1459 | 0 | 0 | 0 | 0 | Discovery/CV | 0 | NA | NA | NA | NA |
| HiSeq1459 | 0 | 1 | 1 | 0 | Discovery/CV | 0 | NA | NA | 0.958456 | −0.11762 |
| HiSeq1459 | 0 | 1 | 1 | 0 | Discovery/CV | 0 | NA | NA | 0.996072 | 0.067383 |
| HiSeq1459 | 0 | 1 | 1 | 0 | Discovery/CV | 0 | NA | NA | 0.515802 | −0.13334 |
| HiSeq1459 | 0 | 1 | 1 | 0 | Discovery/CV | 0 | NA | NA | 0.381863 | 0.019217 |
| HiSeq1459 | 0 | 1 | 1 | 0 | Discovery/CV | 0 | NA | NA | 0.956668 | 0.025275 |
| HiSeq1459 | 0 | 1 | 1 | 0 | Discovery/CV | 0 | NA | NA | 0.084195 | 0.087155 |
| HiSeq1459 | 0 | 1 | 1 | 0 | Discovery/CV | 0 | NA | NA | 0.994532 | 0.224417 |
| HiSeq1459 | 0 | 1 | 1 | 0 | Discovery/CV | 0 | NA | NA | 0.993572 | 0.05598 |
| HiSeq1459 | 0 | 1 | 1 | 0 | Discovery/CV | 0 | NA | NA | 0.480259 | 0.006434 |
| HiSeq1459 | 0 | 1 | 0 | 0 | Discovery/CV | 0 | 0.318863 | NA | 0.130282 | NA |
| HiSeq1459 | 0 | 1 | 0 | 0 | Discovery/CV | 0 | 0.592845 | NA | 0.177272 | NA |
| HiSeq1459 | 0 | 1 | 0 | 0 | Discovery/CV | 0 | 0.17095 | NA | 0.158948 | NA |
| HiSeq1459 | 0 | 1 | 0 | 0 | Discovery/CV | 0 | 0.627652 | NA | 0.204296 | NA |
| HiSeq1459 | 0 | 1 | 0 | 0 | Discovery/CV | 0 | 0.074733 | NA | 0.213747 | NA |
| HiSeq1638 | 0 | 1 | 0 | 0 | Validation | 0 | NA | NA | 0.992994 | −0.02279 |
| HiSeq1638 | 0 | 1 | 0 | 0 | Validation | 0 | NA | NA | 0.999518 | 0.110467 |
| HiSeq1638 | 0 | 1 | 0 | 0 | Validation | 0 | NA | NA | 0.99946 | −0.03009 |
| HiSeq1639 | 0 | 1 | 0 | 0 | Validation | 0 | NA | NA | 0.999648 | 0.132353 |
| HiSeq1639 | 0 | 1 | 0 | 0 | Validation | 0 | NA | NA | 0.85948 | 0.263762 |
| HiSeq1639 | 0 | 1 | 0 | 0 | Validation | 0 | NA | NA | 0.330216 | 0.188967 |
| HiSeq1639 | 0 | 1 | 0 | 0 | Validation | 0 | NA | NA | 0.998037 | 0.538064 |
| HiSeq1639 | 0 | 1 | 0 | 0 | Validation | 0 | NA | NA | 0.963571 | 0.386423 |
| HiSeq1639 | 0 | 1 | 0 | 0 | Validation | 0 | NA | NA | 0.849972 | 0.209177 |
| HiSeq1639 | 0 | 1 | 0 | 0 | Validation | 0 | NA | NA | 0.972781 | 0.470957 |
| HiSeq1639 | 0 | 1 | 0 | 0 | Validation | 0 | NA | NA | 0.540751 | 0.054458 |
| HiSeq1639 | 0 | 1 | 0 | 0 | Validation | 0 | NA | NA | 0.988308 | −0.14027 |
| HiSeq1639 | 0 | 1 | 0 | 0 | Validation | 0 | NA | NA | 0.068826 | 0.31262 |
| HiSeq1639 | 0 | 1 | 0 | 0 | Validation | 0 | NA | NA | 0.985514 | −0.11463 |
| HiSeq1633 | 0 | 0 | 0 | 0 | Validation | 0 | 0.295034 | NA | NA | NA |
| HiSeq1633 | 0 | 0 | 0 | 0 | Validation | 0 | 0.963638 | NA | NA | NA |
| HiSeq1633 | 0 | 0 | 0 | 0 | Validation | 0 | 0.991956 | NA | NA | NA |
| HiSeq1633 | 0 | 0 | 0 | 0 | Validation | 0 | 0.108774 | NA | NA | NA |
| HiSeq1633 | 0 | 0 | 0 | 0 | Validation | 0 | 0.489822 | NA | NA | NA |
| HiSeq1633 | 0 | 0 | 0 | 0 | Validation | 0 | 0.311509 | NA | NA | NA |
| HiSeq1677 | 0 | 0 | 0 | 0 | Validation | 0 | 0.186626 | NA | NA | NA |
| HiSeq1633 | 0 | 0 | 0 | 0 | Validation | 0 | 0.121655 | NA | NA | NA |
| HiSeq1633 | 0 | 0 | 0 | 0 | Validation | 0 | 0.604531 | NA | NA | NA |
| HiSeq1633 | 0 | 0 | 0 | 1 | Validation | 0 | 0.135425 | NA | NA | NA |
| HiSeq1633 | 0 | 1 | 0 | 0 | Validation | 0 | 0.159217 | NA | 0.281583 | NA |
| HiSeq1677 | 0 | 1 | 0 | 0 | Validation | 0 | 0.009032 | NA | 0.027654 | NA |
| HiSeq1633 | 0 | 1 | 0 | 0 | Validation | 0 | 0.604904 | NA | 0.21196 | NA |
| HiSeq1677 | 0 | 0 | 0 | 0 | Validation | 0 | 0.007565 | NA | NA | NA |
| HiSeq1634 | 0 | 0 | 0 | 1 | Validation | 0 | 0.463572 | NA | NA | NA |
| HiSeq1634 | 0 | 0 | 0 | 1 | Validation | 0 | NA | NA | NA | NA |
| HiSeq1634 | 0 | 0 | 0 | 1 | Validation | 0 | 0.973567 | NA | NA | NA |
| HiSeq1634 | 0 | 0 | 0 | 1 | Validation | 0 | NA | NA | NA | NA |
| HiSeq1634 | 0 | 0 | 0 | 1 | Validation | 0 | NA | NA | NA | NA |
| HiSeq1634 | 0 | 0 | 0 | 1 | Validation | 0 | 0.201418 | NA | NA | NA |
| HiSeq1634 | 0 | 0 | 0 | 1 | Validation | 0 | NA | NA | NA | NA |
| HiSeq1634 | 0 | 0 | 0 | 1 | Validation | 0 | 0.887228 | NA | NA | NA |
| HiSeq1634 | 0 | 0 | 0 | 1 | Validation | 0 | NA | NA | NA | NA |
| HiSeq1634 | 0 | 0 | 0 | 1 | Validation | 0 | 0.788946 | NA | NA | NA |
| HiSeq1634 | 0 | 1 | 0 | 0 | Validation | 0 | 0.398429 | NA | 0.021117 | NA |
| HiSeq1634 | 0 | 1 | 0 | 0 | Validation | 0 | 0.037321 | NA | 0.154946 | NA |
| HiSeq1634 | 0 | 1 | 0 | 0 | Validation | 0 | 0.132063 | NA | 0.203212 | NA |
| HiSeq1635 | 0 | 0 | 0 | 0 | Validation | 0 | 0.665496 | NA | NA | NA |
| HiSeq1635 | 0 | 0 | 0 | 0 | Validation | 0 | 0.970346 | NA | NA | NA |
| HiSeq1635 | 0 | 0 | 0 | 1 | Validation | 0 | 0.99667 | NA | NA | NA |
| HiSeq1635 | 0 | 0 | 0 | 1 | Validation | 0 | NA | NA | NA | NA |
| HiSeq1635 | 0 | 0 | 0 | 1 | Validation | 0 | NA | NA | NA | NA |
| HiSeq1635 | 0 | 0 | 0 | 1 | Validation | 0 | NA | NA | NA | NA |
| HiSeq1635 | 0 | 0 | 0 | 1 | Validation | 0 | NA | NA | NA | NA |
| HiSeq1635 | 0 | 0 | 0 | 1 | Validation | 0 | NA | NA | NA | NA |
| HiSeq1635 | 0 | 0 | 0 | 1 | Validation | 0 | 0.998173 | NA | NA | NA |
| HiSeq1635 | 0 | 1 | 0 | 0 | Validation | 0 | 0.128812 | NA | 0.063309 | NA |
| HiSeq1635 | 0 | 1 | 0 | 0 | Validation | 0 | 0.168367 | NA | 0.04024 | NA |
| HiSeq1635 | 0 | 1 | 0 | 0 | Validation | 0 | 0.105187 | NA | 0.078707 | NA |
| HiSeq1635 | 0 | 1 | 0 | 0 | Validation | 0 | 0.015961 | NA | 0.04824 | NA |
| HiSeq1635 | 0 | 1 | 0 | 0 | Validation | 0 | NA | NA | 0.960245 | 0.155203 |
| HiSeq1638 | 0 | 1 | 0 | 0 | Validation | 0 | NA | NA | 0.996971 | 0.098879 |
| HiSeq1638 | 0 | 1 | 0 | 0 | Validation | 0 | NA | NA | 0.999818 | 0.65725 |
| HiSeq1638 | 0 | 1 | 0 | 0 | Validation | 0 | NA | NA | 0.999758 | 0.602011 |
| HiSeq1638 | 0 | 1 | 0 | 0 | Validation | 0 | NA | NA | 0.423205 | 0.221757 |
| HiSeq1638 | 0 | 1 | 0 | 0 | Validation | 0 | NA | NA | 0.992389 | 0.146532 |
| HiSeq1638 | 0 | 1 | 0 | 0 | Validation | 0 | NA | NA | 0.970728 | 0.425999 |
| HiSeq1638 | 0 | 1 | 0 | 0 | Validation | 0 | NA | NA | 0.99971 | 0.060357 |
| HiSeq1638 | 0 | 1 | 0 | 0 | Validation | 0 | 0.007633 | NA | 0.015963 | NA |
| HiSeq1638 | 0 | 1 | 0 | 0 | Validation | 0 | 0.278575 | NA | 0.24419 | NA |
| HiSeq1639 | 0 | 1 | 0 | 0 | Validation | 0 | NA | NA | 0.241977 | 0.25331 |
| HiSeq1639 | 0 | 1 | 0 | 0 | Validation | 0 | 0.043436 | NA | 0.148458 | NA |
| HiSeq1639 | 0 | 1 | 0 | 0 | Validation | 0 | 0.014255 | NA | 0.041533 | NA |
| HiSeq1674 | 0 | 0 | 0 | 1 | Validation | 0 | NA | NA | NA | NA |
| HiSeq1637 | 0 | 1 | 0 | 0 | Validation | 0 | 0.284535 | NA | 0.550669 | NA |
| HiSeq1638 | 0 | 1 | 0 | 0 | Validation | 0 | 0.597303 | NA | 0.131166 | NA |
| HiSeq1639 | 0 | 1 | 0 | 0 | Validation | 0 | 0.119207 | NA | 0.103707 | NA |
| HiSeq1640 | 0 | 1 | 0 | 0 | Validation | 0 | 0.009959 | NA | 0.422806 | NA |
| HiSeq1641 | 0 | 1 | 0 | 0 | Validation | 0 | 0.139247 | NA | 0.201871 | NA |
| HiSeq1645 | 0 | 1 | 0 | 0 | Validation | 0 | 0.581368 | NA | 0.128276 | NA |
| HiSeq1646 | 0 | 1 | 0 | 0 | Validation | 0 | 0.054843 | NA | 0.133745 | NA |
| HiSeq1647 | 0 | 1 | 0 | 0 | Validation | 0 | 0.171725 | NA | 0.239105 | NA |
| HiSeq1649 | 0 | 1 | 0 | 0 | Validation | 0 | 0.020863 | NA | 0.050417 | NA |

[symbol]

indicates data missing or illegible when filed

What is claimed is:

1. A bait set comprising:
a plurality of probes configured to enrich for cell-free DNA molecules from at least 5% of the genomic regions in Table 1 or Table 2.

2. The bait set of claim 1, wherein the plurality of probes are configured to enrich for cell-free DNA molecules from at least 5%, at least 10%, at least 20%, at least 30%, at least 40%, at least 50%, at least 60%, at least 70%, at least 80%, at least 90%, at least 95%, at least 99%, or 100% of the genomic regions in Table 1.

3. The bait set of any preceding claim, wherein at least 20%, at least 30%, at least 40%, at least 50%, at least 60%, at least 70%, at least 80%, at least 90%, at least 95%, or at least 99% of probes in the bait set are configured to enrich for genomic regions in Table 1.

4. The bait set of any preceding claim, wherein the plurality of probes are configured to enrich for cell-free DNA molecules from at least 100, at least 500, at least 1,000, at least 1,500, or at least 2,000 genomic regions in Table 1.

5. The bait set of any preceding claim, wherein each of the plurality of probes comprises a nucleic acid sequence of at least 50 bases, at least 70 bases, at least 80 bases, or at least 100 bases in length that has at least 95%, 99%, or 100% complementarity to a sequence of a region in Table 1.

6. The bait set of claim 1, wherein the plurality of probes is configured to enrich for cell-free DNA molecules from at least 5%, at least 10%, at least 20%, at least 30%, at least 40%, at least 50%, at least 60%, at least 70%, at least 80%, at least 90%, at least 95%, at least 99%, or 100% of the genomic regions in Table 2.

7. The bait set of any one of claims 1 or 6, wherein at least at least 20%, at least 30%, at least 40%, at least 50%, at least 60%, at least 70%, at least 80%, at least 90%, at least 95%, or at least 99% of probes in the bait set are configured to enrich for genomic regions in Table 2.

8. The bait set of any one of claims 1, 6, or 7, wherein the plurality probes are configured to enrich for cell-free DNA molecules from at least 500, at least 1,000, or at least 1,500 genomic regions in Table 2.

9. The bait set of any one of claims 1, 6, 7, or 8, wherein each of the plurality of probes comprises a nucleic acid sequence of at least 50 bases, at least 70, at least 80, or at least 100 bases in length that has at least 95%, 99%, or 100% complementarity to a sequence of a region in Table 2.

10. The bait set of any preceding claim, wherein each of the plurality of probes comprises a nucleic acid sequence configured for hybridization capture of the cell-free DNA molecules.

11. The bait set of claim 10, wherein each of the plurality of probes is at least 50 bases, at least 100 bases, or at least 200 bases in length.

12. The bait set of claim 10 or claim 11, wherein each of the plurality of probes is no more than 500 bases, 1,000 bases, 2,000 bases, or 5,000 bases in length.

13. The bait set of any one of claims 10-12, wherein each of the plurality of probes is between 50 and 5,000 bases, between 100 and 4,000 bases, or between 200 and 2,500 bases, or between 100 and 500 bases in length.

14. The bait set of any preceding claim, wherein the plurality of probes comprises at least 100, at least 500, at least 1000, or at least 4000 different probes.

15. The bait set of any preceding claim, wherein the bait set has at most 10,000 different probes.

16. The bait set of any preceding claim, wherein the plurality of probes collectively extend across portions of the genome that collectively are a combined size of between 0.5 MB and 2.5 MB.

17. The bait set of any preceding claim, wherein each probe of the plurality of probes comprises a pull-down tag.

18. The bait set of claim 17, wherein the pull-down tag comprises biotin.

19. A mixture comprising:
(a) cell-free DNA from a biological sample of a subject; and
(b) the bait set of any one of claims 1-18.

20. The mixture of claim 19, wherein the subject is a human subject.

21. The mixture of claim 19 or claim 20, wherein the biological sample is selected from a blood sample, a serum sample, or a plasma sample.

22. A method for determining by inference an expression level of one or more genes of interest in a subject, the method comprising:
(i) obtaining sequencing data for a plurality of cell-free DNA molecules of a subject;
(ii) aligning the sequencing data for the plurality of cell-free DNA molecules to a reference genome;
(iii) determining sequence length for each of the plurality of cell-free DNA molecules of the subject;
(iv) calculating, for each of the one or more genes of interest, a fragment length diversity measure from cell-free DNA molecules that, when aligned to the reference genome, are within a specified distance from a transcription start site of the gene of interest; and
(v) determining, by inference, a gene expression level for the one or more genes of interest based at least in part on the fragment length diversity measure for each of the one or more genes of interest.

23. The method of claim 22, the method further comprising contacting the cell-free DNA molecules of the subject with the bait set of any one of claims 1-18 to enrich for cell-free DNA from regions within 750 base pairs of transcription start sites.

24. The method of claim 22, wherein the fragment length diversity measure is calculated from cell-free DNA molecules in which both ends fall within 1 kb of the transcription start site for the gene of interest.

25. The method of any one of claims 22-24, wherein the fragment length diversity measure is promoter fragment entropy, wherein promoter fragment entropy is calculated using the equation
$$PFE ( TSS ) := {E}_{k} [ {\sum }_{i : 1 - 5} {P}^{*} ( {e}_{TSS} > ( 1 + k ) \times {e}_{i} ) ] .$$

26. The method of any one of claims 22-25, further comprising calculating a nucleosome depleted region depth.

27. The method of claim 26, further comprising combining the calculated fragment length entropy measure with the calculated nucleosome depleted region depth to generate a metric that is indicative of the expression level of the gene of interest.

28. The method of any one of claims 22-27, wherein steps (iv) and (v) are performed by a computer system comprising software components for data analysis as a program of instructions executable by the computer system.

29. The method of any one of claims 22-28, wherein steps (ii)-(v) are performed by a computer system comprising software components for data analysis as a program of instructions executable by the computer system.

30. The method of any one of claims 22-27, wherein steps (i)-(v) are performed by a computer system comprising software components for data analysis as a program of instructions executable by the computer system.

31. The method of any one of claims 22-30, further comprising:
obtaining a biological sample from the subject, the biological sample comprising the cell-free DNA;
constructing a sequencing library from the cell-free DNA from the biological sample;
sequencing the sequencing library to obtain the sequencing data for the plurality of cell-free DNA molecules of the subject.

32. The method of claim 31, wherein constructing the sequencing library comprises ligating adaptors to the cell-free nucleic acid molecules and enriching for nucleic acids from select regions by hybridizing a selector to the adaptor-containing molecules, thereby forming the sequencing library.

33. The method of claim 32, wherein the selector comprises or consists of a selector as described in the specification.

34. The method of claim 31 or claim 32, wherein selector is designed to enrich for cell-free DNA molecules in proximity to (e.g., within 1 kb of) one or more transcription start sites for one or more genes, wherein the genes are selected from ASCL1, CLDN3, DLL3, DNALI1, DPYSL3, EEF1A2, ESRP1, FOXA2, GRP, HOXB5, ID4, IGFBP5, IGFBPL1, ISL1, KRT19, KRT7, MMP2, NKX2-1, PCSK2, SCG3, SIX1, SYT13, SYT4, TAGLN3, and TM4SF1.

35. The method of claim 34, wherein the selector is designed to enrich for cell-free DNA molecules in proximity to transcription start sites for at least 10%, at least 20%, at least 50%, at least 70%, at least 80%, at least 90, at least 95%, or 100% of the following genes: ASCL1, CLDN3, DLL3, DNALI1, DPYSL3, EEF1A2, ESRP1, FOXA2, GRP, HOXB5, ID4, IGFBP5, IGFBPL1, ISL1, KRT19, KRT7, MMP2, NKX2-1, PCSK2, SCG3, SIX1, SYT13, SYT4, TAGLN3, and TM4SF1.

36. The method of any one of claims 31-33, wherein the biological sample is obtained from an individual with cancer.

37. The method of claim 36, wherein the cancer is a cancer described in the specification.

38. The method of claim 37, wherein the cancer is small cell lung cancer.

39. The method of claim 37, wherein the cancer non-small cell lung cancer.

40. The method of claim 37Error! Reference source not found., wherein the cancer is lung cancer or a B-cell lymphoma.

41. The method of any one of claims 36-40, wherein the subject has a tumor burden having a mixture fraction of at least 0.25, 0.5, 0.75, 1.0, 1.5, 2.0, 2.5, 5, 7.5, 10, or 15 and the sequencing data has at least 500×, 2500×, or 5000× coverage for regions comprising the transcription start sites for the one or more genes of interest.

42. The method of any one of claims 22-41, wherein the sequencing data is obtained from a biological sample obtained prior to immune checkpoint inhibitor treatment.

43. The method of any one of claims 22-42, wherein gene expression levels for the one or more genes of interest are monitored after treatment with an immune checkpoint inhibitor.

44. The method of any one of claims 22-43, wherein the sequencing data is obtained from a biological sample that was obtained within 4 weeks of a first immune checkpoint inhibitor treatment.

45. The method of any one of claims 36-44, wherein the individual with cancer (1) is treated with an immune checkpoint inhibitor if durable clinical benefit is predicted and (2) is treated with non-immune checkpoint inhibitor therapy if durable clinical benefit is not predicted.

46. The method of any one of claims 42-45, wherein the immune checkpoint inhibitor is a PD-1 or PD-L1 inhibitor.

47. The method of any one of claims 36-46, wherein if the individual is diagnosed as having a specific cancer, said individual is then treated for said cancer.

48. The method of any one of claims 31-47, wherein the biological sample is a non-invasively obtained sample from blood.

49. The method of claim 48, wherein the biological sample is a serum sample.

50. The method of any one of claims 31-47, wherein the sequencing is at a depth of at least 500×, 2000×, 2500× or 5000×.

51. The method of any one of claims 22-50, wherein an increase in the fragment length diversity measure (e.g., promoter fragment entropy) of the gene of interest correlates with an increase in expression of the gene of interest.

52. The method of any one of claims 22-51, wherein an increase in the fragment length diversity measure (e.g., promoter fragment entropy) of the gene of interest correlates with expression of exon 1 of the gene of interest.

53. The method of any one of claims 22-52, further comprising identifying the subject as having a disease state based at least in part on (1) the fragment length diversity measure of a plurality of genes of interest or (2) the gene expression levels of the plurality of genes of interest as determined by inference from the fragment length diversity measures for the plurality of genes.

54. The method of any one of claims 22-53, further comprising identifying a tissue of origin for diseased tissue from the subject based at least in part on (1) the fragment length diversity measure of a plurality of genes of interest or (2) the gene expression levels of the plurality of genes of interest as determined by inference from the fragment length diversity measures for the plurality of genes.

55. The method of any one of claims 22-54, wherein the number of genes of interest is at least two, at least 5, at least 10, at least 15, or at least 25.

56. The method of any one of claims 22-55, wherein one or more steps are implemented on a computer system comprising a software component configured for analysis of data obtained by the methods.

57. The method of any one of claims 22-56, further comprising:
obtaining a biological sample from the subject, the biological sample comprising the cell-free DNA;
constructing a sequencing library from the cell-free DNA from the biological sample;
sequencing the sequencing library to obtain the sequencing data for the plurality of cell-free DNA molecules of the subject.

58. The method of claim 57, wherein constructing the sequencing library comprises enriching for cell-free nucleic acid molecules from select regions by hybridization capture.

59. The method of claim 57, wherein constructing the sequencing library comprises ligating adaptors to the cell-free nucleic acid molecules and enriching for nucleic acids from select regions by hybridizing a selector to the adaptor-containing molecules, thereby forming the sequencing library.

60. The method of claim 59, wherein the selector comprises or consists of a selector as described in the specification.

61. The method of claim 59 or claim 60, wherein selector comprises or consists of the bait set of any one of claims 1-18.

62. A software product tangibly embodied in a machine-readable medium, the software product comprising instructions operable to cause one or more data processing apparatuses to perform the method of any of claims 22-61.

63. A method for determining a fragment length diversity measure for one or more genes of interest, the method comprising:
(i) obtaining sequencing data for a plurality of cell-free DNA molecules of a subject;
(ii) aligning the sequencing data for the plurality of cell-free DNA molecules to a reference genome;
(iii) determining sequence length for each of the plurality of cell-free DNA molecules of the subject; and
(iv) calculating, for each of the one or more genes of interest, a fragment length diversity measure from cell-free DNA molecules that, when aligned to the reference genome, are within a specified distance from a transcription start site of the gene of interest.

64. The method of claim 63, the method further comprising contacting the cell-free DNA molecules of the subject with the bait set of any one of claims 1-18 to enrich for cell-free DNA from regions within 750 base pairs of transcription start sites.

65. The method of claim 63 or claim 64, wherein the fragment length diversity measure is calculated from cell-free DNA molecules in which both ends fall within 1 kb of the transcription start site for the gene of interest.

66. The method of claim 63 or claim 65, wherein the fragment length diversity measure is calculated from cell-free DNA molecules in which both ends fall within 900 base pairs, within 850 pairs, within 800 base pairs, or within 750 base pairs of the transcription start site for the gene of interest.

67. The method of any one of claims 63-66, wherein the fragment length diversity measure is promoter fragment entropy, wherein promoter fragment entropy is calculated using the equation PFE(TSS):=Eₖ[Σ_{i:1-5}P*(e_{TSS}>(1+k)×eᵢ)].

68. The method of any one of claims 63-67, further comprising calculating a nucleosome depleted region depth.

69. The method of claim 68, further comprising combining the calculated fragment length entropy measure with the calculated nucleosome depleted region depth to generate a metric that is indicative of an expression level of the gene of interest.

70. The method of any one of claims 63-69, wherein steps (iii) and (iv) are performed by a computer system comprising software components for data analysis as a program of instructions executable by the computer system.

71. The method of any one of claims 63-70, wherein steps (ii)-(iv) are performed by a computer system comprising software components for data analysis as a program of instructions executable by the computer system.

72. The method of any one of claims 63-71, wherein steps (i)-(iv) are performed by a computer system comprising software components for data analysis as a program of instructions executable by the computer system.

73. The method of any one of claims 63-72, further comprising:
obtaining a biological sample from the subject, the biological sample comprising the cell-free DNA;
constructing a sequencing library from the cell-free DNA from the biological sample;
sequencing the sequencing library to obtain the sequencing data for the plurality of cell-free DNA molecules of the subject.

74. The method of claim 73, the method further comprising contacting the cell-free DNA molecules of the subject with the bait set of any one of claims 1-18 to enrich for cell-free DNA from regions within 750 base pairs of transcription start sites.

75. The method of claim 73, wherein constructing the sequencing library comprises ligating adaptors to the cell-free nucleic acid molecules.

76. The method of claim 73, wherein constructing the sequencing library comprises enriching for cell-free nucleic acid molecules from select regions by hybridization capture.

77. The method of claim 73, wherein constructing the sequencing library comprises ligating adaptors to the cell-free nucleic acid molecules and enriching for nucleic acids from select regions by hybridizing a selector to the adaptor-containing molecules, thereby forming the sequencing library.

78. The method of claim 77, wherein the selector comprises or consists of a selector as described in the specification.

79. The method of claim 77 or claim 78, wherein the selector comprises or consists of the bait set of any one of claims 1-18.

80. The method of claim 77 or claim 78, wherein selector is designed to enrich for cell-free DNA molecules in proximity to (e.g., within 1 kb of) one or more transcription start sites for one or more genes, wherein the genes are selected from ASCL1, CLDN3, DLL3, DNALI1, DPYSL3, EEF1A2, ESRP1, FOXA2, GRP, HOXB5, ID4, IGFBP5, IGFBPL1, ISL1, KRT19, KRT7, MMP2, NKX2-1, PCSK2, SCG3, SIX1, SYT13, SYT4, TAGLN3, and TM4SF1.

81. The method of claim 80, wherein the selector is designed to enrich for cell-free DNA molecules in proximity to transcription start sites for at least 10%, at least 20%, at least 50%, at least 70%, at least 80%, at least 90, at least 95%, or 100% of the following genes: ASCL1, CLDN3, DLL3, DNALI1, DPYSL3, EEF1A2, ESRP1, FOXA2, GRP, HOXB5, ID4, IGFBP5, IGFBPL1, ISL1, KRT19, KRT7, MMP2, NKX2-1, PCSK2, SCG3, SIX1, SYT13, SYT4, TAGLN3, and TM4SF1.

82. The method of any one of claims 73-78, wherein the biological sample is obtained from an individual with cancer.

83. The method of claim 82, wherein the cancer is a cancer described in the specification.

84. The method of claim 83, wherein the cancer is small cell lung cancer.

85. The method of claim 83, wherein the cancer non-small cell lung cancer.

86. The method of claim 83, wherein the cancer is lung cancer or a B-cell lymphoma.

87. The method of any one of claims 82-86, wherein the subject has a tumor burden having a mixture fraction of at least 0.25, 0.5, 0.75, 1.0, 1.5, 2.0, 2.5, 5, 7.5, 10, or 15 and the sequencing data has at least 500×, 2500×, or 5000× coverage for regions comprising the transcription start sites for the one or more genes of interest.

88. The method of any one of claims 63-87, wherein the sequencing data is obtained from a biological sample obtained prior to immune checkpoint inhibitor treatment.

89. The method of any one of claims 63-88, further comprising calculating, for each of the one or more genes of interest, a fragment length diversity after treatment with an immune checkpoint inhibitor.

90. The method of any one of claims 63-89, wherein the sequencing data is obtained from a biological sample that was obtained within 4 weeks of a first immune checkpoint inhibitor treatment.

91. The method of any one of claims 82-90, wherein the individual with cancer (1) is treated with an immune checkpoint inhibitor if durable clinical benefit is predicted and (2) is treated with non-immune checkpoint inhibitor therapy if durable clinical benefit is not predicted.

92. The method of any one of claims 88-91, wherein the immune checkpoint inhibitor is a PD-1 or PD-L1 inhibitor.

93. The method of any one of claims 83-92, wherein if the individual is diagnosed as having a specific cancer, said individual is then treated for said cancer.

94. The method of any one of claims 73-93, wherein the biological sample is a non-invasively obtained sample from blood.

95. The method of claim 94, wherein the biological sample is a serum sample.

96. The method of any one of claims 73-93, wherein the sequencing is at a depth of at least 500×, 2000×, 2500× or 5000×.

97. The method of any one of claims 63-96, wherein an increase in the fragment length diversity measure (e.g., promoter fragment entropy) of the gene of interest correlates with an increase in expression of the gene of interest.

98. The method of any one of claims 63-97, wherein an increase in the fragment length diversity measure (e.g., promoter fragment entropy) of the gene of interest correlates with expression of exon 1 of the gene of interest.

99. The method of any one of claims 63-98, further comprising identifying a tissue of origin for diseased tissue from the subject based at least in part on (1) the fragment length diversity measure of a plurality of genes of interest or (2) the gene expression levels of the plurality of genes of interest as determined by inference from the fragment length diversity measures for the plurality of genes.

100. The method of any one of claims 63-99, wherein the number of genes of interest is at least two, at least 5, at least 10, at least 15, or at least 25.

101. The method of any one of claims 63-100, wherein one or more steps are implemented on a computer system comprising a software component configured for analysis of data obtained by the methods.

102. A software product tangibly embodied in a machine-readable medium, the software product comprising instructions operable to cause one or more data processing apparatuses to perform the method of any one of claims 63-101.
